(19) United States
(12) Patent Application Publication
(10) Pub. No.: US 2025/0136585 A1
(43) Pub. Date: May 1, 2025

(54) HETEROCYCLIC COMPOUND, ORGANIC LIGHT-EMITTING DEVICE COMPRISING SAME, AND COMPOSITION FOR ORGANIC LAYER

(71) Applicant: LT MATERIALS CO., LTD., Yongin-si, Gyeonggi-do (KR)

(72) Inventors: Young Beom SEO, Yongin-si (KR); Hyun Joo LEE, Yongin-si (KR); Young Seok NO, Yongin-si (KR); Dong Jun KIM, Yongin-si (KR)

(73) Assignee: LT MATERIALS CO., LTD., Yongin-si, Gyeonggi-do (KR)

(21) Appl. No.: 18/834,865

(22) PCT Filed: Jan. 2, 2023
(86) PCT No.: PCT/KR2023/000018

§ 371 Date: Jul. 31, 2024

(30) Foreign Application Priority Data
Feb. 4, 2022 (KR) 10-2022-0014538

(51) Int. Cl. C07D 405/14; C07B 59/00; C09K 11/02; C09K 11/06; H10K 85/40; H10K 85/60

(52) U.S. Cl. CPC C07D 405/14; C07B 59/004; C09K 11/02; C09K 11/06; H10K 85/40; H10K 85/615; H10K 85/622; H10K 85/654; H10K 85/6572; H10K 85/6574; C07B 2200/05

(57) ABSTRACT

The present disclosure relates to a heterocyclic compound represented by Chemical Formula 1 and an organic light emitting device including the same.

### TECHNICAL FIELD

This application claims priority to and the benefits of Korean Patent Application No. 10-2022-0014538, filed with the Korean Intellectual Property Office on Feb. 4, 2022, the entire contents of which are incorporated herein by reference. The present disclosure relates to a heterocyclic compound, an organic light emitting device comprising the same, and a composition for an organic material layer.

### BACKGROUND ART

An organic light emitting device is one type of self-emissive display devices, and has advantages of having a wide viewing angle and a high response speed as well as having an excellent contrast.

The organic light emitting device has a structure of disposing an organic thin film between two electrodes. When a voltage is applied to the organic light emitting device having such a structure, electrons and holes injected from the two electrodes bind and pair in the organic thin film, and then light emits as these annihilate. The organic thin film may be formed in a single layer or a multilayer as necessary.

A material of the organic thin film may have a light emitting function as necessary. For example, as a material of the organic thin film, compounds each capable of forming a light emitting layer themselves alone may be used, or compounds each capable of performing a role of a host or a dopant of a host-dopant-based light emitting layer may also be used. In addition thereto, compounds capable of performing roles of hole injection, hole transport, electron blocking, hole blocking, electron transport, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

U.S. Pat. No. 4,356,429

### DISCLOSURE

### Technical Problem

An object of present disclosure is to provide a heterocyclic compound, an organic light emitting device comprising the same, and a composition for an organic material layer.

### Technical Solution

In order to achieve the object, one embodiment of the present disclosure provides a heterocyclic compound represented by the following Chemical Formula 1.

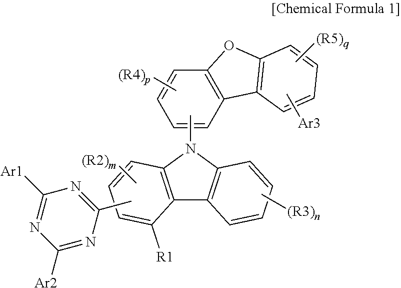

In Chemical Formula 1,
R1 is hydrogen; or deuterium,
R2 to R5 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
Ar1 to Ar3 are the same as or different from each other, and each independently a substituted or unsubstituted C6 to C60 aryl group; or a substituted or unsubstituted C2 to C60 heteroaryl group,
m is an integer of 0 to 2, and when m is 2 or greater, R2s are the same as or different from each other,
n is an integer of 0 to 4, and when n is 2 or greater, R3s are the same as or different from each other,
p is an integer of 0 to 3, and when p is 2 or greater, R4s are the same as or different from each other, and
q is an integer of 0 to 3, and when q is 2 or greater, R5s are the same as or different from each other.

In addition, the present disclosure provides an organic light emitting device comprising:
a first electrode;
a second electrode provided opposite to the first electrode; and
one or more organic material layers provided between the first electrode and the second electrode,
wherein at least one of the one or more organic material layers comprise the heterocyclic compound represented by Chemical Formula 1.

In addition, the present disclosure provides the organic light emitting device, wherein the organic material layer further comprises a heterocyclic compound represented by the following Chemical Formula 2.

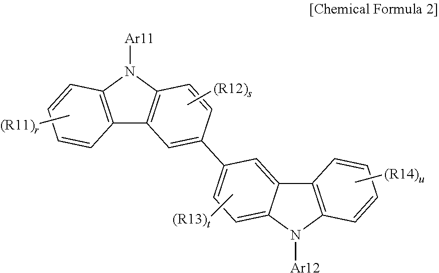

In Chemical Formula 2,
R11 to R14 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; and —NR201R202, or two or more groups adjacent to each other bond to each other to form a substituted or unsubstituted C6 to C60 aromatic hydrocarbon ring or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group,
Ar11 and Ar12 are the same as or different from each other, and each independently a substituted or unsubstituted C6 to C60 aryl group; or a substituted or unsubstituted C2 to C60 heteroaryl group,
r is an integer of 0 to 4, and when r is 2 or greater, R11s are the same as or different from each other,
s is an integer of 0 to 3, and when s is 2 or greater, R12s are the same as or different from each other,
t is an integer of 0 to 3, and when t is 2 or greater, R13s are the same as or different from each other, and
u is an integer of 0 to 4, and when u is 2 or greater, R14s are the same as or different from each other.

In addition, the present disclosure provides a composition for an organic material layer, the composition comprising: the heterocyclic compound represented by Chemical Formula 1; and the heterocyclic compound represented by Chemical Formula 2.

### Advantageous Effects

A compound described in the present specification can be used as a material of an organic material layer of an organic light emitting device. The compound is capable of performing roles of a hole injection layer material, a hole transport layer material, a light emitting layer material, an electron transport layer material, an electron injection layer material and the like in an organic light emitting device. Particularly, the compound can be used as a light emitting layer material of an organic light emitting device, and the compound can be used as a light emitting material either alone or as a host material or a dopant material of the light emitting layer.

Specifically, the compound can be used as a light emitting material either alone or as a host material or a dopant material of the light emitting layer. Using the heterocyclic compound represented by Chemical Formula 1 in an organic material layer is capable of lowering a driving voltage, enhancing light emission efficiency and enhancing lifetime properties in an organic light emitting device.

### DESCRIPTION OF DRAWINGS

FIGS. 1 to 4 are diagrams each schematically illustrating a lamination structure of an organic light emitting device according to one embodiment of the present disclosure.

### MODE FOR DISCLOSURE

Hereinafter, the present disclosure will be described in more detail.

In the present specification, a term “substitution” means that a hydrogen atom bonding to a carbon atom of a compound is changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, “substituted or unsubstituted” means being substituted with one or more substituents selected from the group consisting of deuterium; halogen; a cyano group; C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; —P(═O)RR′; C1 to C20 alkylamine; C6 to C60 monocyclic or polycyclic arylamine; and C2 to C60 monocyclic or polycyclic heteroarylamine or being unsubstituted, or being substituted with a substituent in which two or more substituents selected from among the substituents exemplified above are linked or being unsubstituted.

In the present specification, the halogen may be fluorine, chlorine, bromine or iodine.

In the present specification, the alkyl group includes linear or branched having 1 to 60 carbon atoms, and may be further substituted with other substituents. The number of carbon atoms of the alkyl group may be from 1 to 60, specifically from 1 to 40 and more specifically from 1 to 20. Specific examples thereof may include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2, 2-dimethylheptyl group, a 1-ethyl-propyl group, a 1,1-dimethyl-propyl group, an isohexyl group, a 4-methylhexyl group, a 5-methylhexyl group and the like, but are not limited thereto.

In the present specification, the alkenyl group includes linear or branched having 2 to 60 carbon atoms, and may be further substituted with other substituents. The number of carbon atoms of the alkenyl group may be from 2 to 60, specifically from 2 to 40 and more specifically from 2 to 20. Specific examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group includes linear or branched having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples thereof may include a methoxy group, an ethoxy group, an n-propoxy group, an isopropoxy group, an n-butoxy group, an isobutoxy group, a tert-butoxy group, a sec-butoxy group, an n-pentyloxy group, a neopentyloxy group, an isopentyloxy group, an n-hexyloxy group, a 3, 3-dimethylbutyloxy group, a 2-ethylbutyloxy group, an n-octyloxy group, an n-nonyloxy group, an n-decyloxy group, a benzyloxy group, a p-methylbenzyloxy group and the like, but are not limited thereto.

In the present specification, the cycloalkyl group includes monocyclic or polycyclic having 3 to 60 carbon atoms, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon atoms of the cycloalkyl group may be from 3 to 60, specifically from 3 to 40 and more specifically from 5 to 20. Specific examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes monocyclic or polycyclic having 2 to 60 carbon atoms, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group may include a spiro group. The number of carbon atoms of the aryl group may be from 6 to 60, specifically from 6 to 40 and more specifically from 6 to 25. Specific examples of the aryl group may include a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring group thereof, and the like, but are not limited thereto.

In the present specification, the phosphine oxide group is represented by —P(═O)R101R102, and R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specifically, the phosphine oxide group may be substituted with an aryl group, and as the aryl group, the examples described above may be applied. Examples of the phosphine oxide group may include a diphenylphosphine oxide group, a dinaphthylphosphine oxide group and the like, but are not limited thereto.

In the present specification, the silyl group is a substituent including Si and having the Si atom directly linked as a radical, and is represented by —SiR101R102R103. R101 to R103 are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted,

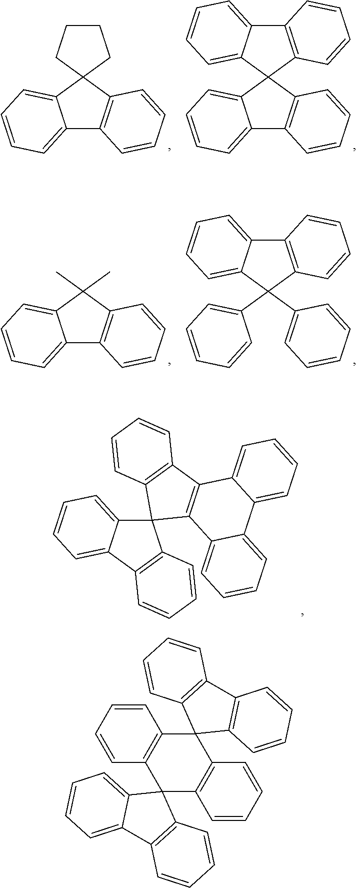

and the like may be included, however, the structure is not limited thereto.

In the present specification, the spiro group is a group including a spiro structure, and may have 15 to 60 carbon atoms. For example, the spiro group may include a structure in which a 2,3-dihydro-1H-indene group or a cyclohexane group spiro bonds to a fluorenyl group. Specifically, the spiro group may include any one of groups of the following structural formulae.

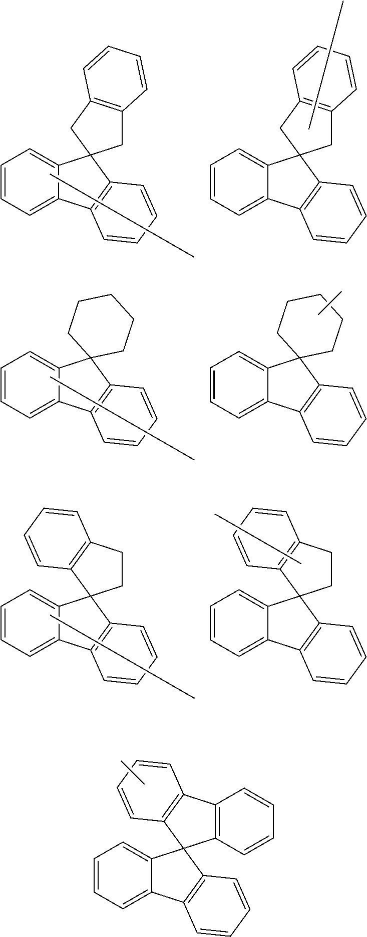

In the present specification, the heteroaryl group includes S, O, Se, N or Si as a heteroatom, includes monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may include a pyridyl group, a pyrrolyl group, a pyrimidyl group, a pyridazinyl group, a furanyl group, a thiophenyl group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolinyl group, a naphthyridyl group, an acridinyl group, a phenanthridinyl group, a diazanaphthalenyl group, a triazaindenyl group, an indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl group, a dibenzocarbazolyl group, a phenazinyl group, a dibenzosilole group, a spirobi (dibenzosilole) group, a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepinyl group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms is not particularly limited, but preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, an “adjacent” group may mean a substituent substituting an atom directly linked to an atom substituted by the corresponding substituent, a substituent sterically closely positioned to the corresponding substituent, another substituent substituting an atom substituted by the corresponding substituent. For example, two substituents substituting at ortho positions in a benzene ring, and two substituents substituting at the same carbon in an aliphatic ring may be interpreted as groups “adjacent” to each other.

In the present disclosure, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present disclosure, a “case of a substituent being not indicated in a chemical formula or compound structure” may mean that positions that may come as a substituent are all hydrogen or deuterium. In other words, since deuterium is an isotope of hydrogen, some hydrogen atoms may be deuterium that is an isotope, and herein, a content of the deuterium may be from 0% to 100%.

In one embodiment of the present disclosure, in a “case of a substituent being not indicated in a chemical formula or compound structure”, hydrogen and deuterium may be used interchangeably in compounds when deuterium is not explicitly excluded such as “a deuterium content being 0%”, “a hydrogen content being 100%” or “substituents being all hydrogen”.

In one embodiment of the present disclosure, deuterium is one of isotopes of hydrogen, is an element having deuteron formed with one proton and one neutron as a nucleus, and may be expressed as hydrogen-2, and the elemental symbol thereof may also be written as D or ²H.

In one embodiment of the present disclosure, an isotope means an atom with the same atomic number (Z) but with a different mass number (A), and may also be interpreted as an element with the same number of protons but with a different number of neutrons.

In one embodiment of the present disclosure, a meaning of a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

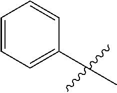 [SMILES: *c1ccccc1]

may mean that the total number of substituents that the phenyl group may have is 5 (T1 in the formula), and the number of deuterium among these is 1 (T2 in the formula). In other words, having a deuterium content of 20% in a phenyl group may be represented by the following structural formulae.

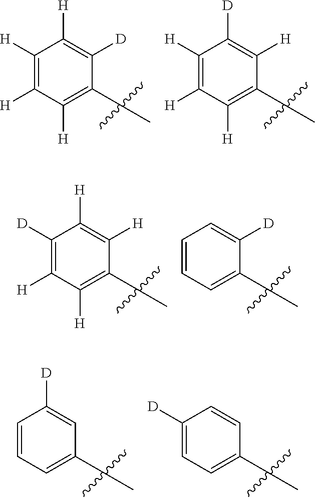 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present disclosure, “a phenyl group having a deuterium content of 0%” may mean a phenyl group that does not include a deuterium atom, that is, a phenyl group that has 5 hydrogen atoms.

In the present disclosure, the C6 to C60 aromatic hydrocarbon ring means a compound including an aromatic ring formed with C6 to C60 carbons and hydrogens. Examples thereof may include benzene, biphenyl, triphenyl, triphenylene, naphthalene, anthracene, phenalene, phenanthrene, fluorene, pyrene, chrysene, perylene, azulene and the like, but are not limited thereto, and include all aromatic hydrocarbon ring compounds known in the art and satisfying the above-mentioned number of carbon atoms.

One embodiment of the present disclosure provides a heterocyclic compound represented by the following Chemical Formula 1.

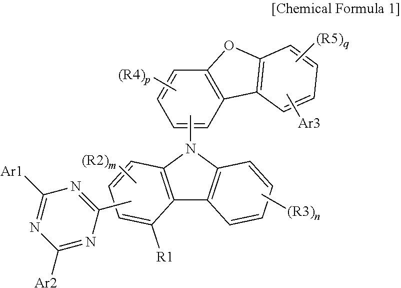

In Chemical Formula 1,
R1 is hydrogen; or deuterium,
R2 to R5 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,

Ar1 to Ar3 are the same as or different from each other, and each independently a substituted or unsubstituted C6 to C60 aryl group; or a substituted or unsubstituted C2 to C60 heteroaryl group,
m is an integer of 0 to 2, and when m is 2 or greater, R2s are the same as or different from each other,
n is an integer of 0 to 4, and when n is 2 or greater, R3s are the same as or different from each other,
p is an integer of 0 to 3, and when p is 2 or greater, R4s are the same as or different from each other, and
q is an integer of 0 to 3, and when q is 2 or greater, R5s are the same as or different from each other.

In one embodiment of the present disclosure, R2 to R5 are the same as or different from each other, and each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102; —SiR101R102R103; or —NR101R102, or two or more groups adjacent to each other bond to each other to form a substituted or unsubstituted C6 to C30 aromatic hydrocarbon ring; or a substituted or unsubstituted C2 to C30 heteroring, and R101, R102 and R103 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present disclosure, R2 to R5 are the same as or different from each other, and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; —SiR101R102R103; or —NR101R102, or two or more groups adjacent to each other bond to each other to form a substituted or unsubstituted C6 to C20 aromatic hydrocarbon ring; or a substituted or unsubstituted C2 to C20 heteroring, and R101, R102 and R103 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R2 to R5 are the same as or different from each other, and may be each independently hydrogen; deuterium; a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R2 to R5 are the same as or different from each other, and may be each independently hydrogen; or deuterium.

In one embodiment of the present disclosure, Ar1 to Ar3 are the same as or different from each other, and may be each independently a substituted or unsubstituted C6 to C30 aryl group; or a substituted or unsubstituted C2 to C30 heteroaryl group.

In another embodiment of the present disclosure, Ar1 to Ar3 are the same as or different from each other, and may be each independently a substituted or unsubstituted C6 to C20 aryl group; or a substituted or unsubstituted C2 to C20 heteroaryl group.

In another embodiment of the present disclosure, Ar1 and Ar2 are the same as or different from each other, and may be each independently a substituted or unsubstituted C6 to C20 aryl group; or a substituted or unsubstituted C2 to C20 heteroaryl group.

In another embodiment of the present disclosure, Ar1 and Ar2 are the same as or different from each other, and may be each independently a substituted or unsubstituted phenyl group; a substituted or unsubstituted biphenyl group; a substituted or unsubstituted fluorenyl group; or a substituted or unsubstituted dibenzofuranyl group.

In another embodiment of the present disclosure, Ar3 may be a substituted or unsubstituted C6 to C20 aryl group.

In another embodiment of the present disclosure, Ar3 may be a substituted or unsubstituted phenyl group; a substituted or unsubstituted biphenyl group; or a substituted or unsubstituted naphthyl group.

In one embodiment of the present disclosure, the heterocyclic compound represented by Chemical Formula 1 may not include deuterium as a substituent, or may have a deuterium content of, for example, greater than 0%, 1% or greater, 10% or greater, 20% or greater, 30% or greater, 40% or greater or 50% or greater, and 100% or less, 90% or less, 80% or less, 70% or less or 60% or less with respect to the total number of hydrogen atoms and deuterium atoms.

In another embodiment of the present disclosure, the heterocyclic compound represented by Chemical Formula 1 may not include deuterium as a substituent, or may have a deuterium content of 1% to 100% with respect to the total number of hydrogen atoms and deuterium atoms.

In another embodiment of the present disclosure, the heterocyclic compound represented by Chemical Formula 1 may not include deuterium as a substituent, or may have a deuterium content of 20% to 90% with respect to the total number of hydrogen atoms and deuterium atoms.

In another embodiment of the present disclosure, the heterocyclic compound represented by Chemical Formula 1 may not include deuterium as a substituent, or may have a deuterium content of 30% to 80% with respect to the total number of hydrogen atoms and deuterium atoms.

In another embodiment of the present disclosure, the heterocyclic compound represented by Chemical Formula 1 may not include deuterium as a substituent, or may have a deuterium content of 50% to 70% with respect to the total number of hydrogen atoms and deuterium atoms.

In one embodiment of the present disclosure, the heterocyclic compound represented by Chemical Formula 1 may be a heterocyclic compound represented by any one of Chemical Formulae 1-1 to 1-4.

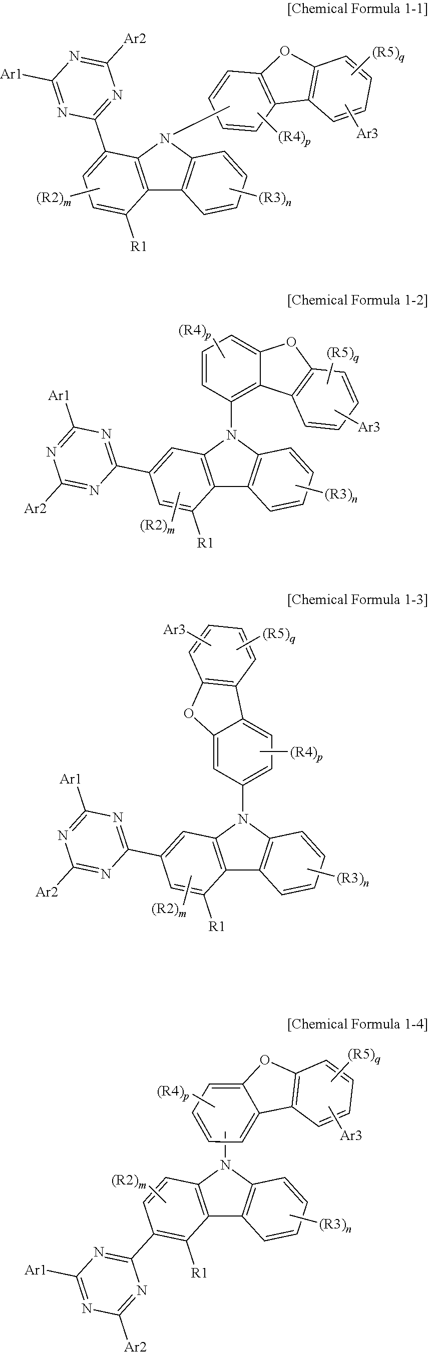

In Chemical Formulae 1-1 to 1-4,
R1 to R5, Ar1 to Ar3, m, n, p and q have the same definitions as in Chemical Formula 1.

In one embodiment of the present disclosure, the heterocyclic compound represented by Chemical Formula 1 may be represented by any one of the following compounds.

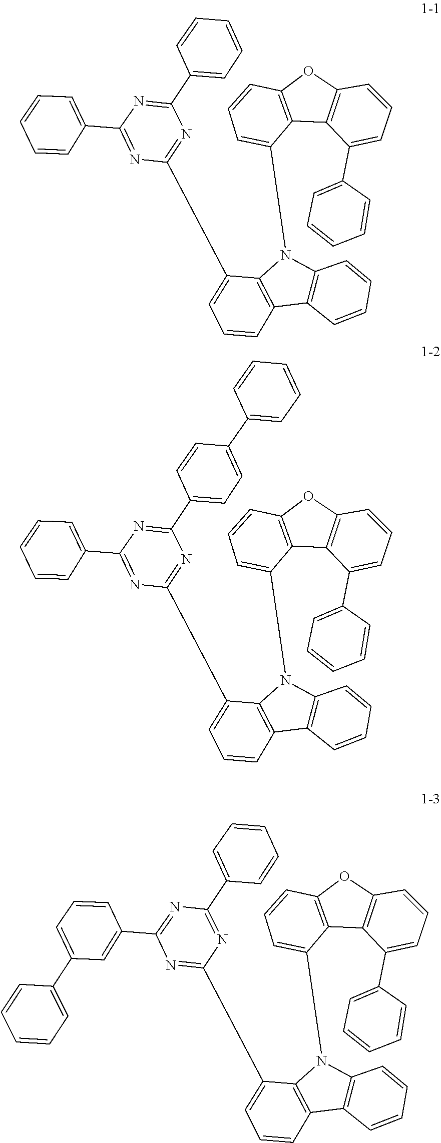

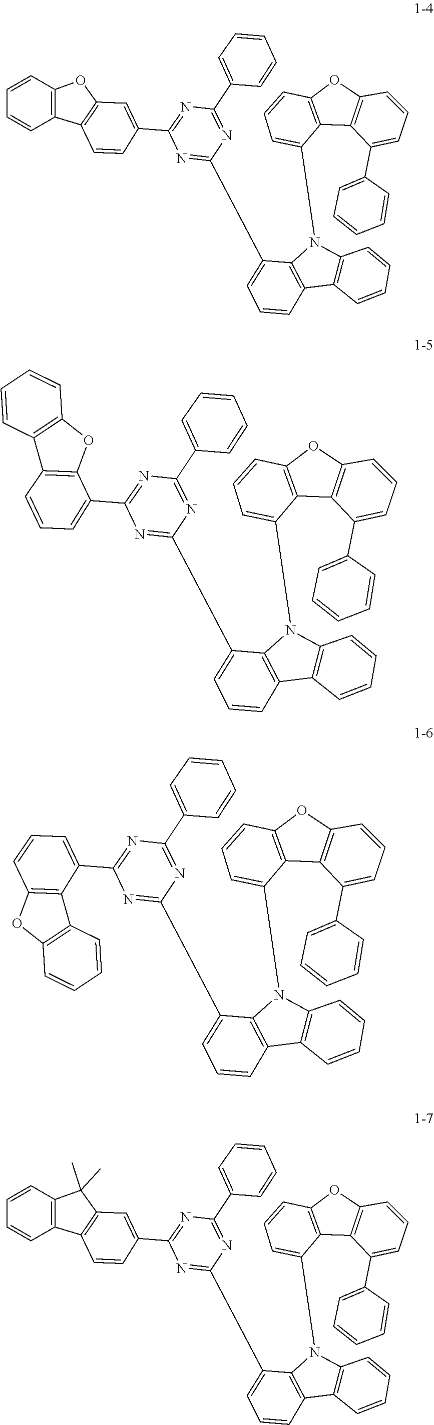 [SMILES: c1ccc(-c2nc(-c3ccc4c(c3)oc3ccccc34)nc(-c3cccc4c5ccccc5n(-c5cccc6oc7cccc(-c8ccccc8)c7c56)c34)n2)cc1 ; c1ccc(-c2nc(-c3cccc4c3oc3ccccc34)nc(-c3cccc4c5ccccc5n(-c5cccc6oc7cccc(-c8ccccc8)c7c56)c34)n2)cc1 ; c1ccc(-c2nc(-c3cccc4oc5ccccc5c34)nc(-c3cccc4c5ccccc5n(-c5cccc6oc7cccc(-c8ccccc8)c7c56)c34)n2)cc1 ; CC1(C)c2ccccc2-c2ccc(-c3nc(-c4ccccc4)nc(-c4cccc5c6ccccc6n(-c6cccc7oc8cccc(-c9ccccc9)c8c67)c45)n3)cc21]

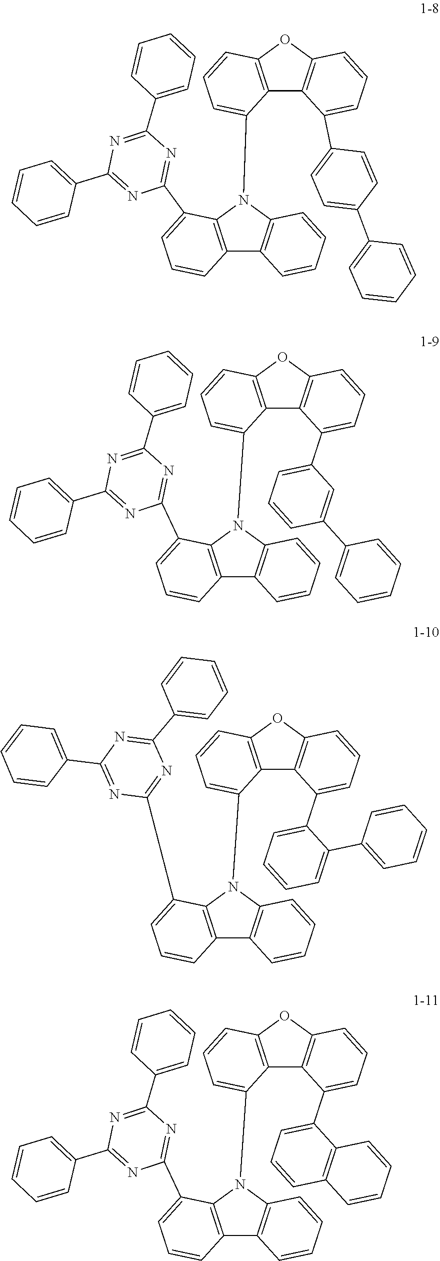 [SMILES: c1ccc(-c2ccc(-c3cccc4oc5cccc(-n6c7ccccc7c7cccc(-c8nc(-c9ccccc9)nc(-c9ccccc9)n8)c76)c5c34)cc2)cc1 ; c1ccc(-c2cccc(-c3cccc4oc5cccc(-n6c7ccccc7c7cccc(-c8nc(-c9ccccc9)nc(-c9ccccc9)n8)c76)c5c34)c2)cc1 ; c1ccc(-c2nc(-c3ccccc3)nc(-c3cccc4c5ccccc5n(-c5cccc6oc7cccc(-c8ccccc8-c8ccccc8)c7c56)c34)n2)cc1 ; c1ccc(-c2nc(-c3ccccc3)nc(-c3cccc4c5ccccc5n(-c5cccc6oc7cccc(-c8cccc9ccccc89)c7c56)c34)n2)cc1]

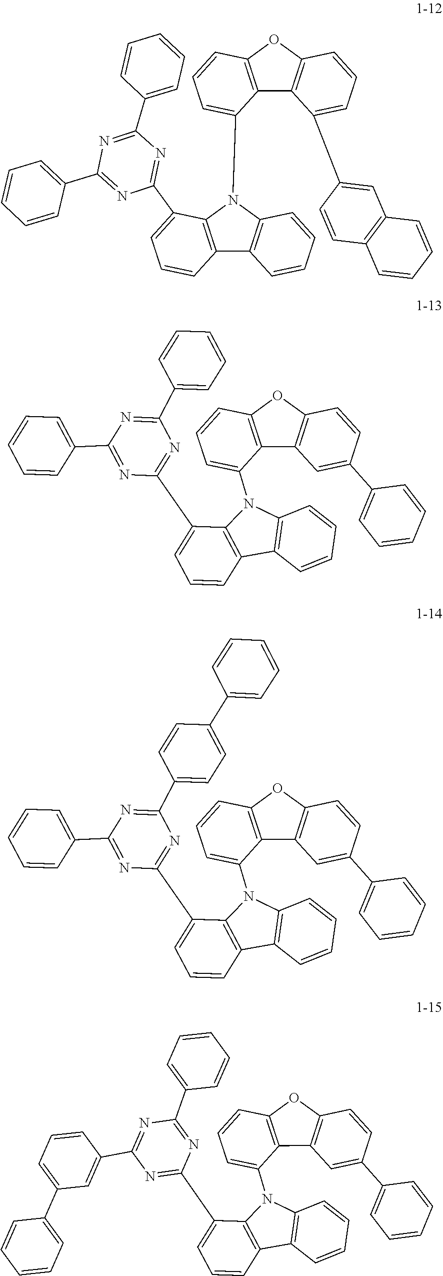 [SMILES: c1ccc(-c2nc(-c3ccccc3)nc(-c3cccc4c5ccccc5n(-c5cccc6oc7cccc(-c8ccc9ccccc9c8)c7c56)c34)n2)cc1 ; c1ccc(-c2ccc3oc4cccc(-n5c6ccccc6c6cccc(-c7nc(-c8ccccc8)nc(-c8ccccc8)n7)c65)c4c3c2)cc1 ; c1ccc(-c2ccc(-c3nc(-c4ccccc4)nc(-c4cccc5c6ccccc6n(-c6cccc7oc8ccc(-c9ccccc9)cc8c67)c45)n3)cc2)cc1 ; c1ccc(-c2cccc(-c3nc(-c4ccccc4)nc(-c4cccc5c6ccccc6n(-c6cccc7oc8ccc(-c9ccccc9)cc8c67)c45)n3)c2)cc1]

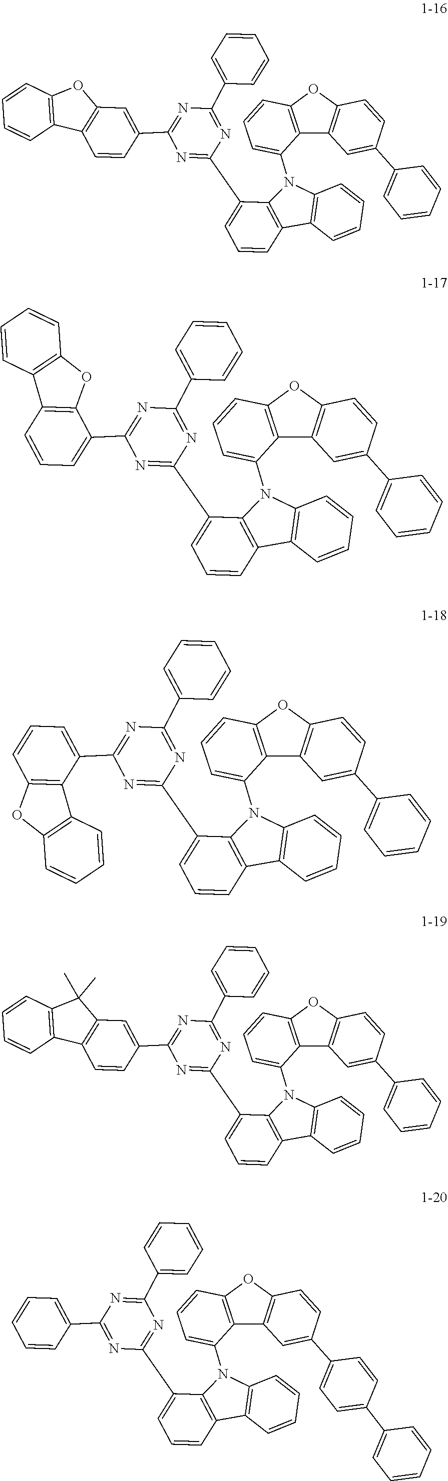

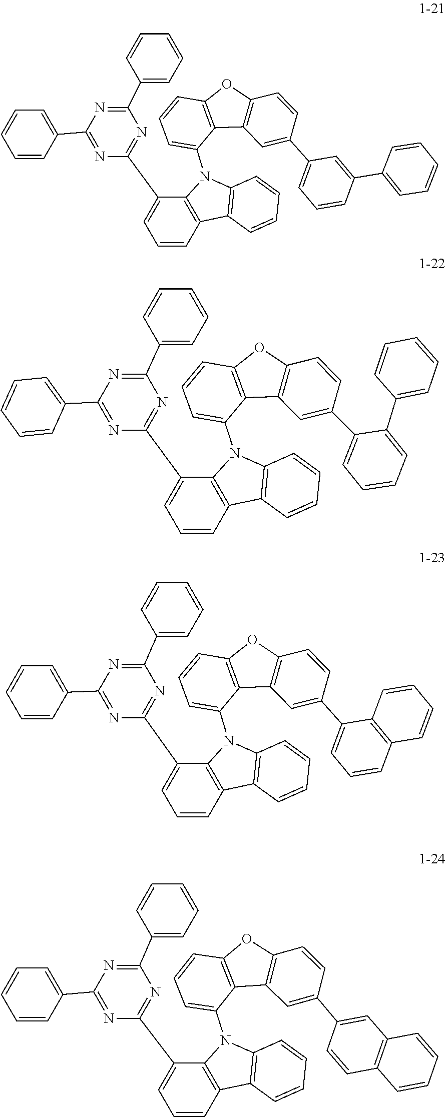

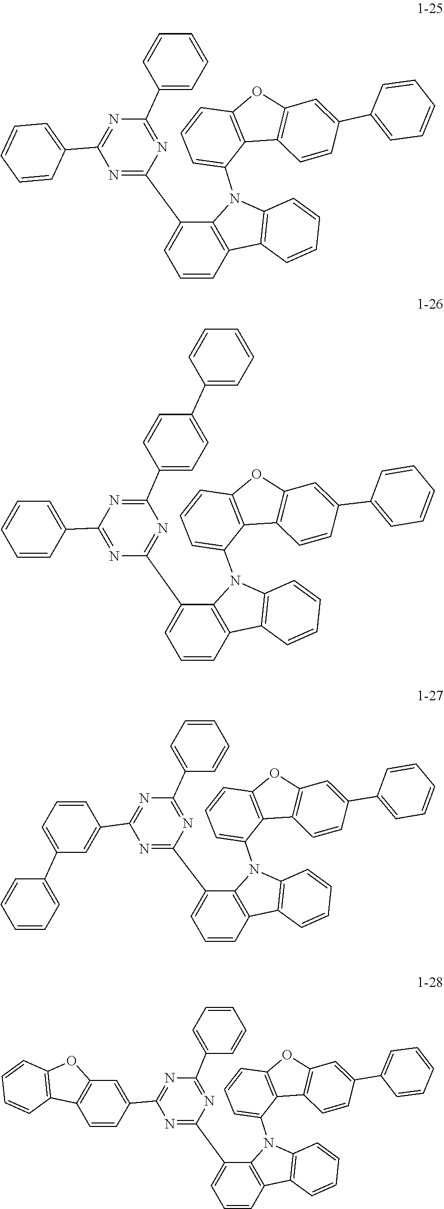 [SMILES: c1ccc(-c2ccc3c(c2)oc2cccc(-n4c5ccccc5c5cccc(-c6nc(-c7ccccc7)nc(-c7ccccc7)n6)c54)c23)cc1 ; c1ccc(-c2ccc(-c3nc(-c4ccccc4)nc(-c4cccc5c6ccccc6n(-c6cccc7oc8cc(-c9ccccc9)ccc8c67)c45)n3)cc2)cc1 ; c1ccc(-c2cccc(-c3nc(-c4ccccc4)nc(-c4cccc5c6ccccc6n(-c6cccc7oc8cc(-c9ccccc9)ccc8c67)c45)n3)c2)cc1 ; c1ccc(-c2ccc3c(c2)oc2cccc(-n4c5ccccc5c5cccc(-c6nc(-c7ccccc7)nc(-c7ccc8c(c7)oc7ccccc78)n6)c54)c23)cc1]

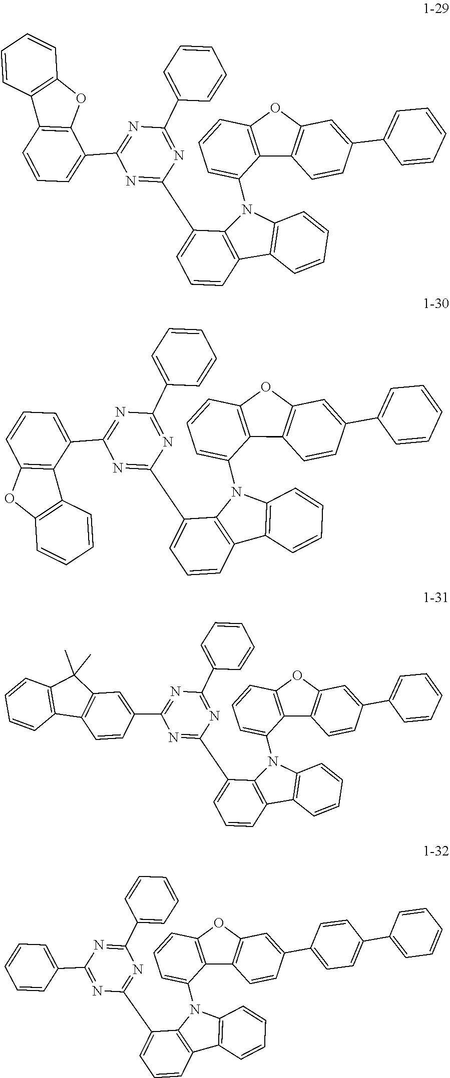 [SMILES: c1ccc(-c2ccc3c(c2)oc2cccc(-n4c5ccccc5c5cccc(-c6nc(-c7ccccc7)nc(-c7cccc8c7oc7ccccc78)n6)c54)c23)cc1 ; c1ccc(-c2ccc3c(c2)oc2cccc(-n4c5ccccc5c5cccc(-c6nc(-c7ccccc7)nc(-c7cccc8oc9ccccc9c78)n6)c54)c23)cc1 ; CC1(C)c2ccccc2-c2ccc(-c3nc(-c4ccccc4)nc(-c4cccc5c6ccccc6n(-c6cccc7oc8cc(-c9ccccc9)ccc8c67)c45)n3)cc21 ; c1ccc(-c2ccc(-c3ccc4c(c3)oc3cccc(-n5c6ccccc6c6cccc(-c7nc(-c8ccccc8)nc(-c8ccccc8)n7)c65)c34)cc2)cc1]

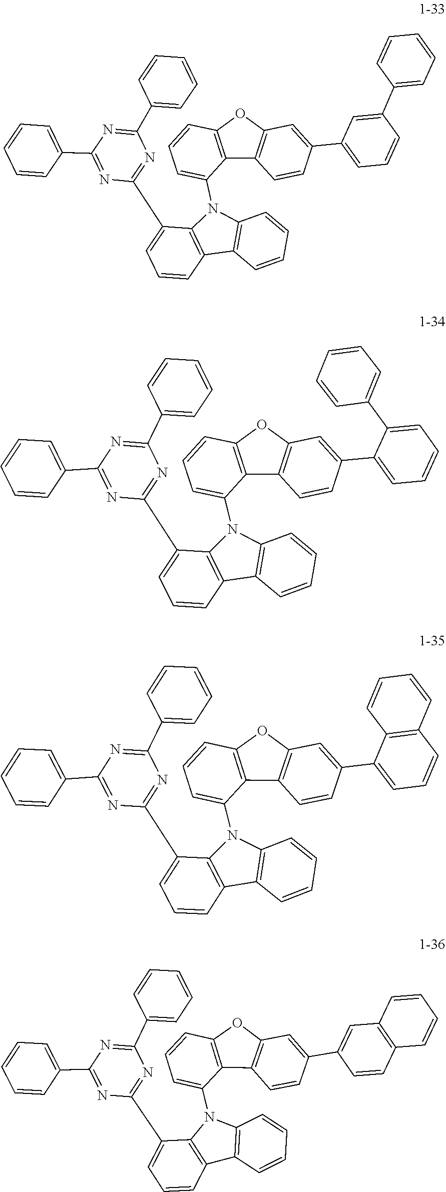 [SMILES: c1ccc(-c2cccc(-c3ccc4c(c3)oc3cccc(-n5c6ccccc6c6cccc(-c7nc(-c8ccccc8)nc(-c8ccccc8)n7)c65)c34)c2)cc1 ; c1ccc(-c2nc(-c3ccccc3)nc(-c3cccc4c5ccccc5n(-c5cccc6oc7cc(-c8ccccc8-c8ccccc8)ccc7c56)c34)n2)cc1 ; c1ccc(-c2nc(-c3ccccc3)nc(-c3cccc4c5ccccc5n(-c5cccc6oc7cc(-c8cccc9ccccc89)ccc7c56)c34)n2)cc1 ; c1ccc(-c2nc(-c3ccccc3)nc(-c3cccc4c5ccccc5n(-c5cccc6oc7cc(-c8ccc9ccccc9c8)ccc7c56)c34)n2)cc1]

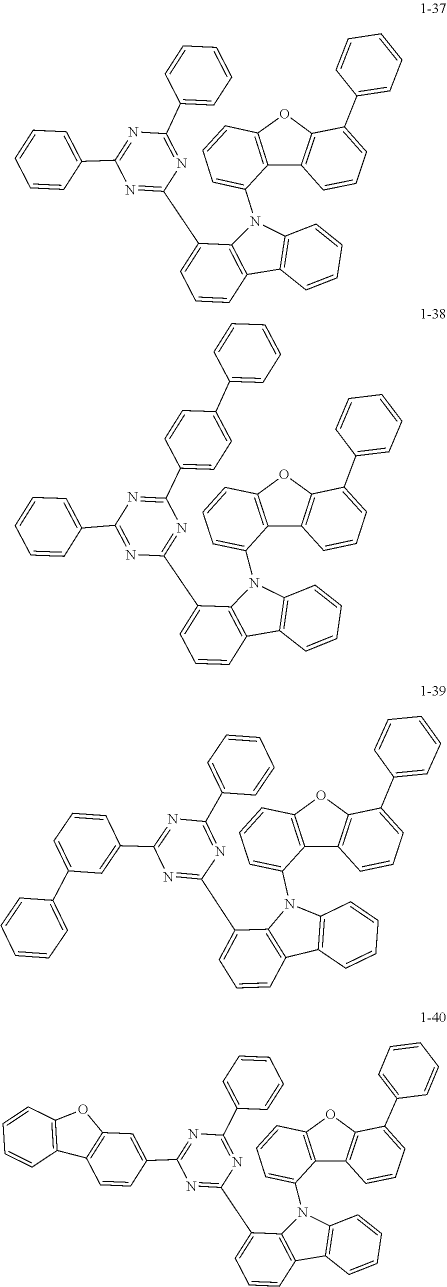

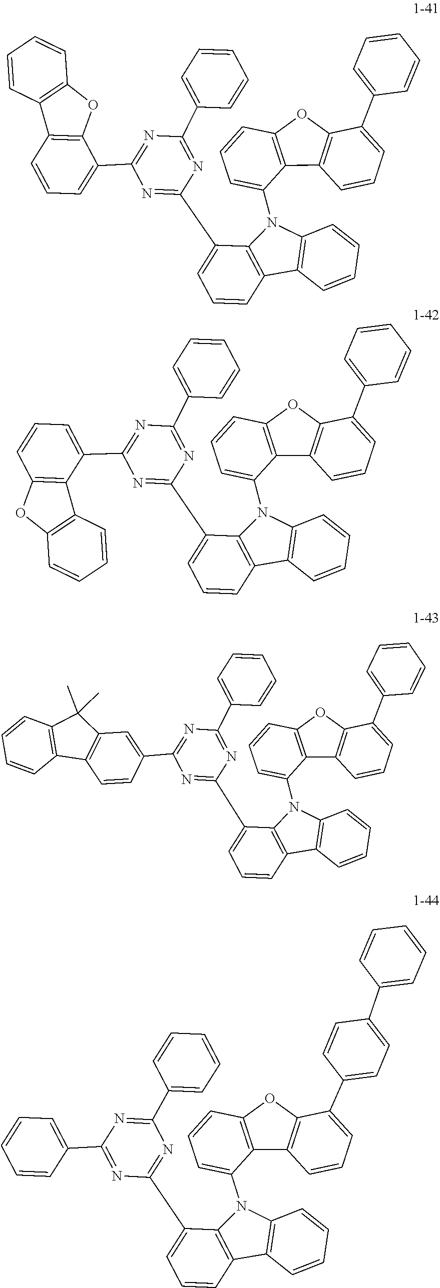

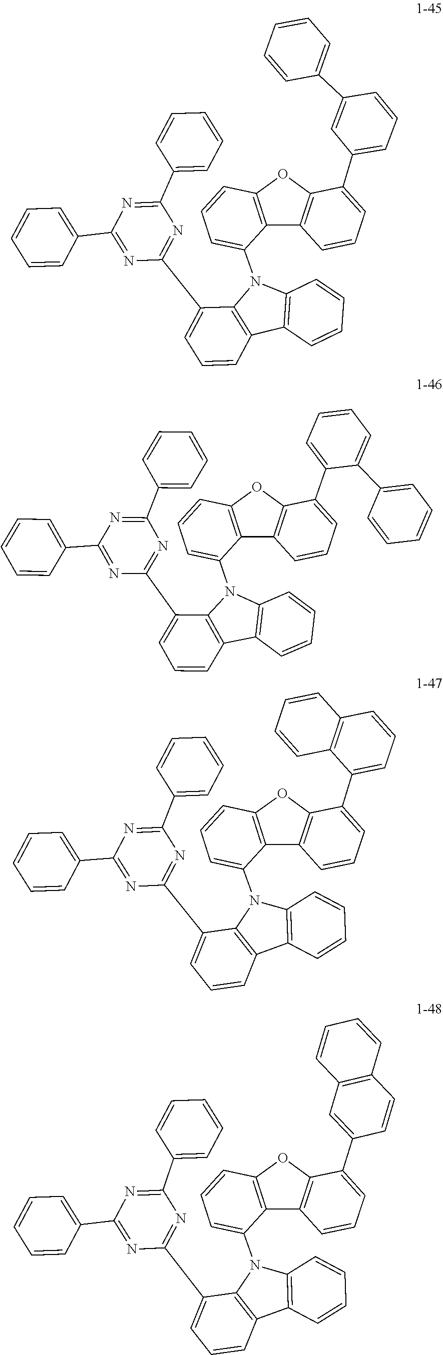

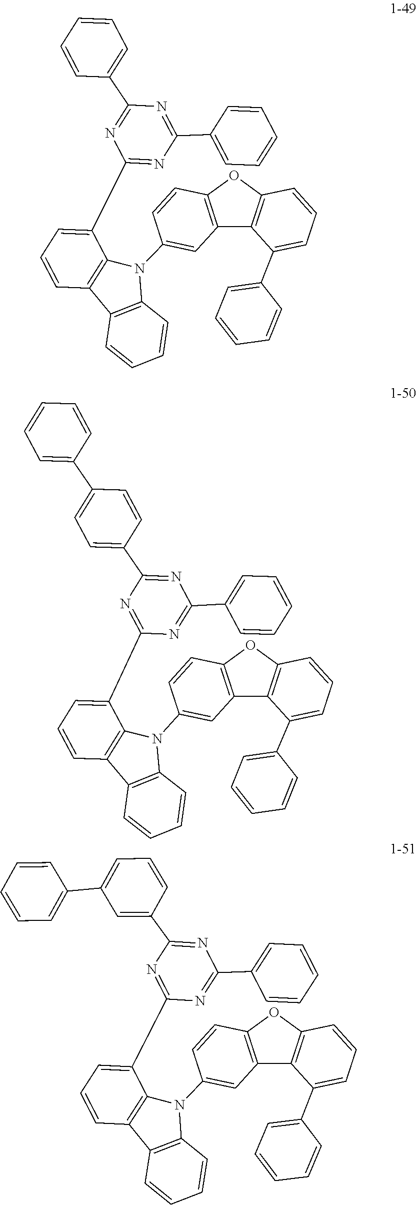

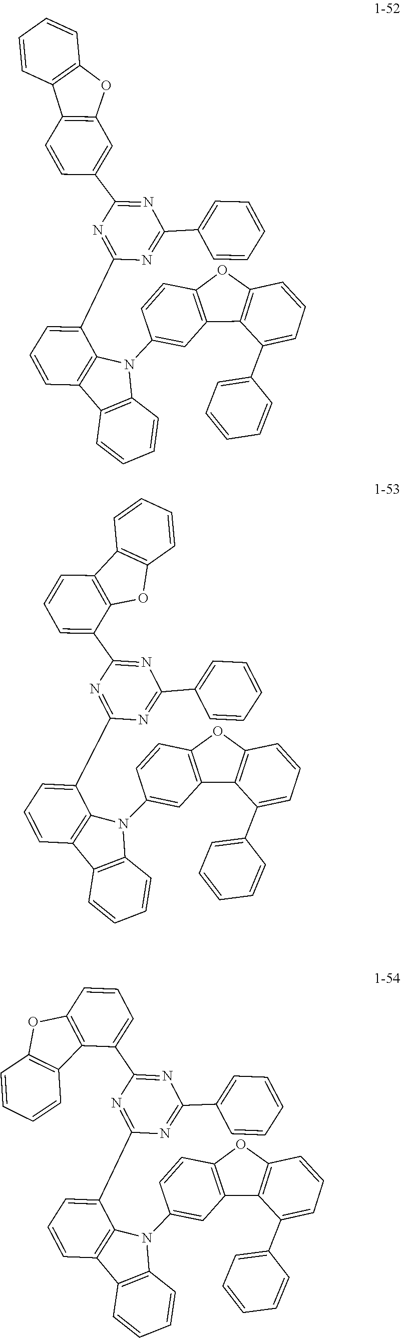

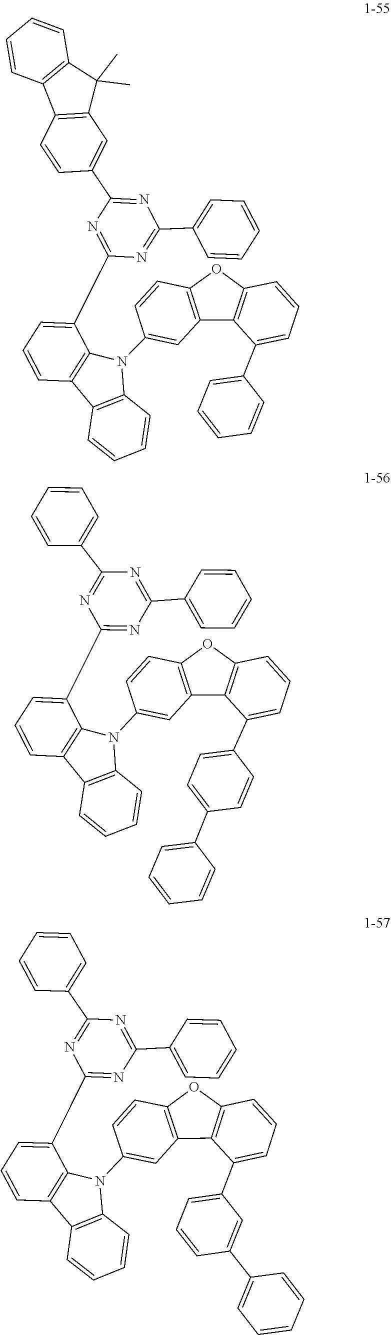

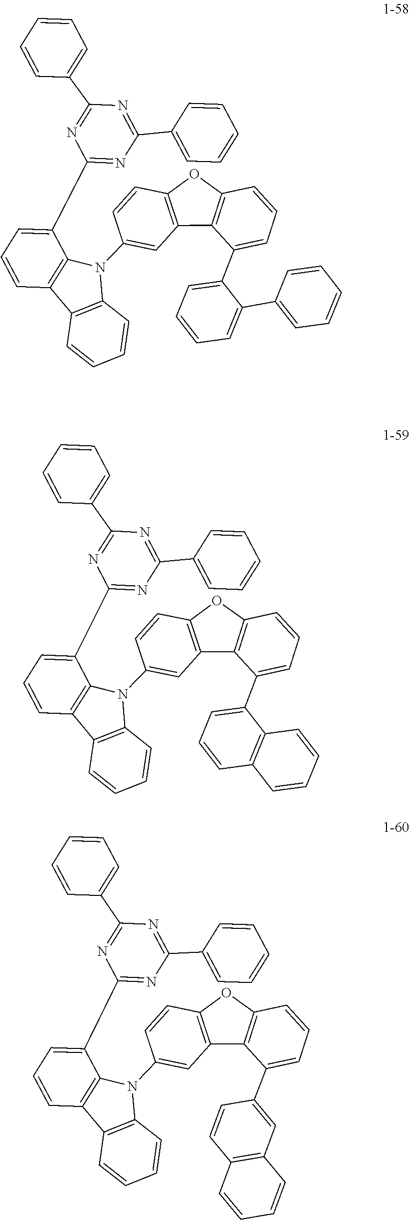

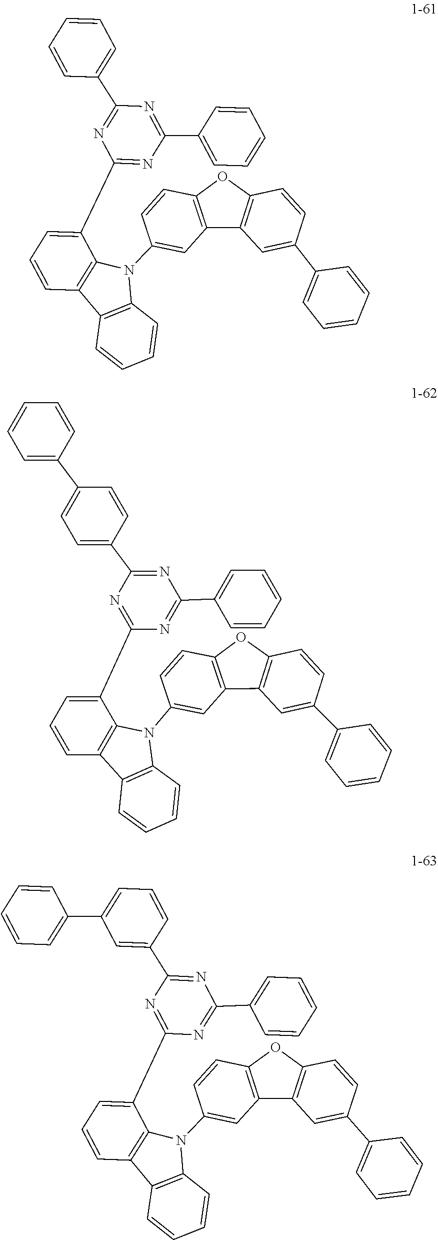 [SMILES: c1ccc(-c2ccc3oc4ccc(-n5c6ccccc6c6cccc(-c7nc(-c8ccccc8)nc(-c8ccccc8)n7)c65)cc4c3c2)cc1 ; c1ccc(-c2ccc(-c3nc(-c4ccccc4)nc(-c4cccc5c6ccccc6n(-c6ccc7oc8ccc(-c9ccccc9)cc8c7c6)c45)n3)cc2)cc1 ; c1ccc(-c2cccc(-c3nc(-c4ccccc4)nc(-c4cccc5c6ccccc6n(-c6ccc7oc8ccc(-c9ccccc9)cc8c7c6)c45)n3)c2)cc1]

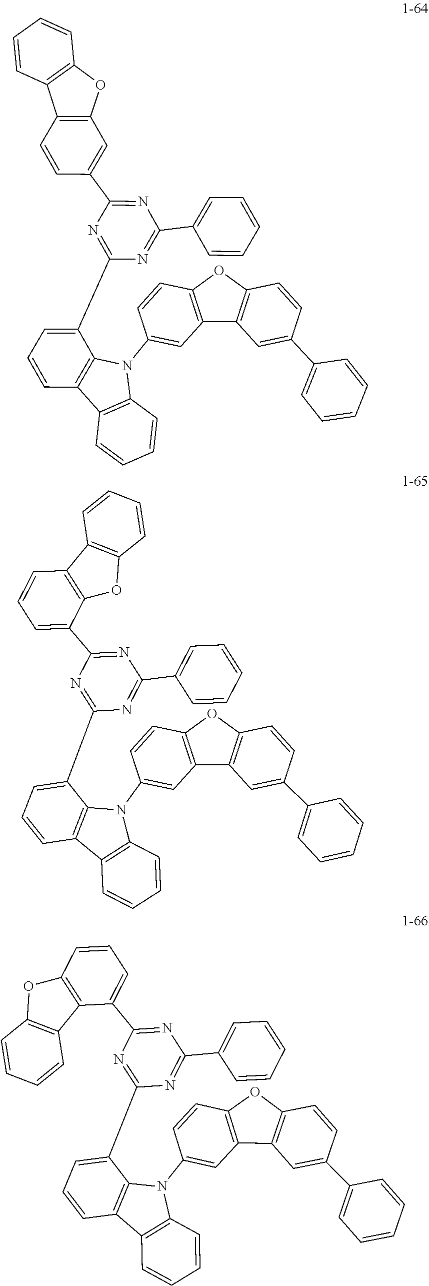

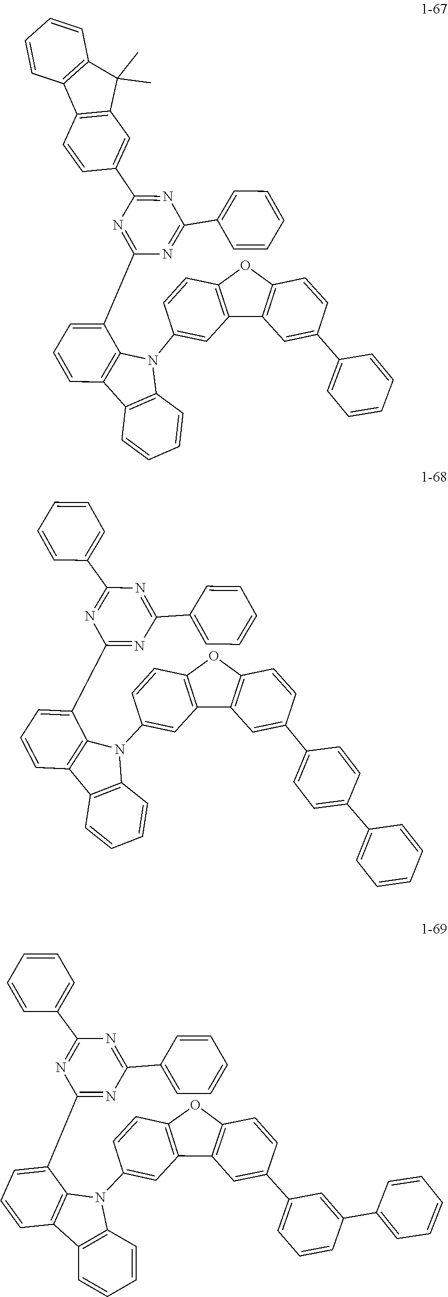 [SMILES: CC1(C)c2ccccc2-c2ccc(-c3nc(-c4ccccc4)nc(-c4cccc5c6ccccc6n(-c6ccc7oc8ccc(-c9ccccc9)cc8c7c6)c45)n3)cc21 ; c1ccc(-c2ccc(-c3ccc4oc5ccc(-n6c7ccccc7c7cccc(-c8nc(-c9ccccc9)nc(-c9ccccc9)n8)c76)cc5c4c3)cc2)cc1 ; c1ccc(-c2cccc(-c3ccc4oc5ccc(-n6c7ccccc7c7cccc(-c8nc(-c9ccccc9)nc(-c9ccccc9)n8)c76)cc5c4c3)c2)cc1]

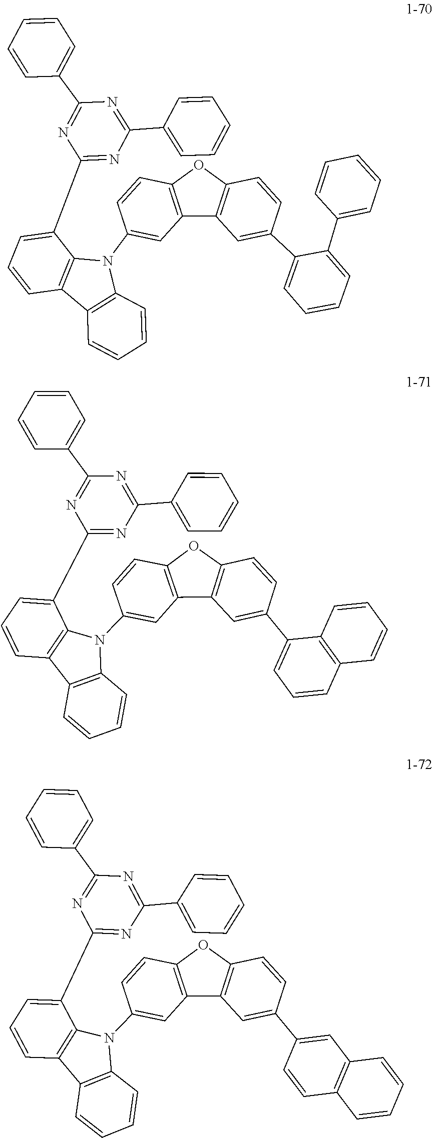 [SMILES: c1ccc(-c2nc(-c3ccccc3)nc(-c3cccc4c5ccccc5n(-c5ccc6oc7ccc(-c8ccccc8-c8ccccc8)cc7c6c5)c34)n2)cc1 ; c1ccc(-c2nc(-c3ccccc3)nc(-c3cccc4c5ccccc5n(-c5ccc6oc7ccc(-c8cccc9ccccc89)cc7c6c5)c34)n2)cc1 ; c1ccc(-c2nc(-c3ccccc3)nc(-c3cccc4c5ccccc5n(-c5ccc6oc7ccc(-c8ccc9ccccc9c8)cc7c6c5)c34)n2)cc1]

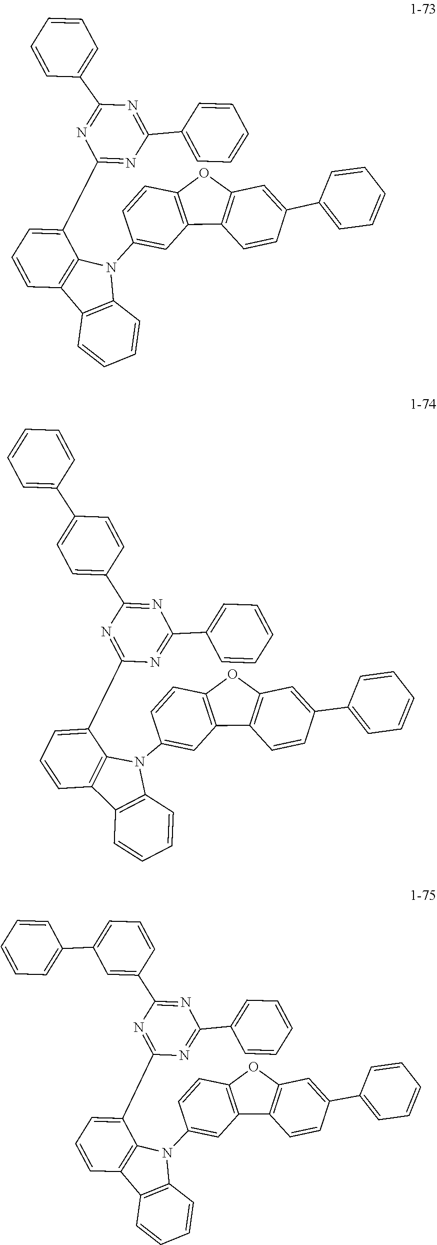 [SMILES: c1ccc(-c2ccc3c(c2)oc2ccc(-n4c5ccccc5c5cccc(-c6nc(-c7ccccc7)nc(-c7ccccc7)n6)c54)cc23)cc1 ; c1ccc(-c2ccc(-c3nc(-c4ccccc4)nc(-c4cccc5c6ccccc6n(-c6ccc7oc8cc(-c9ccccc9)ccc8c7c6)c45)n3)cc2)cc1 ; c1ccc(-c2cccc(-c3nc(-c4ccccc4)nc(-c4cccc5c6ccccc6n(-c6ccc7oc8cc(-c9ccccc9)ccc8c7c6)c45)n3)c2)cc1]

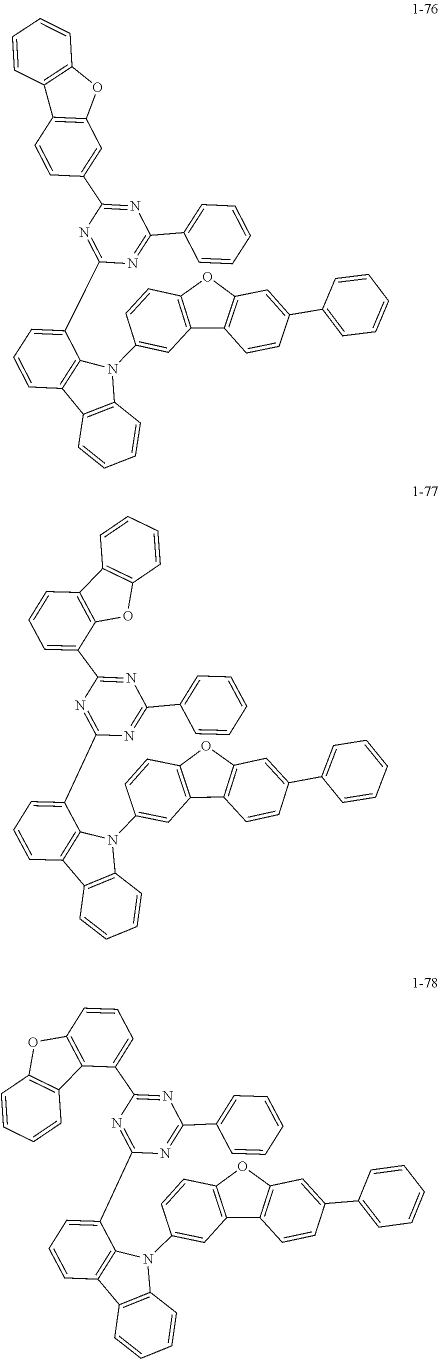 [SMILES: c1ccc(-c2ccc3c(c2)oc2ccc(-n4c5ccccc5c5cccc(-c6nc(-c7ccccc7)nc(-c7ccc8c(c7)oc7ccccc78)n6)c54)cc23)cc1 ; c1ccc(-c2ccc3c(c2)oc2ccc(-n4c5ccccc5c5cccc(-c6nc(-c7ccccc7)nc(-c7cccc8c7oc7ccccc78)n6)c54)cc23)cc1 ; c1ccc(-c2ccc3c(c2)oc2ccc(-n4c5ccccc5c5cccc(-c6nc(-c7ccccc7)nc(-c7cccc8oc9ccccc9c78)n6)c54)cc23)cc1]

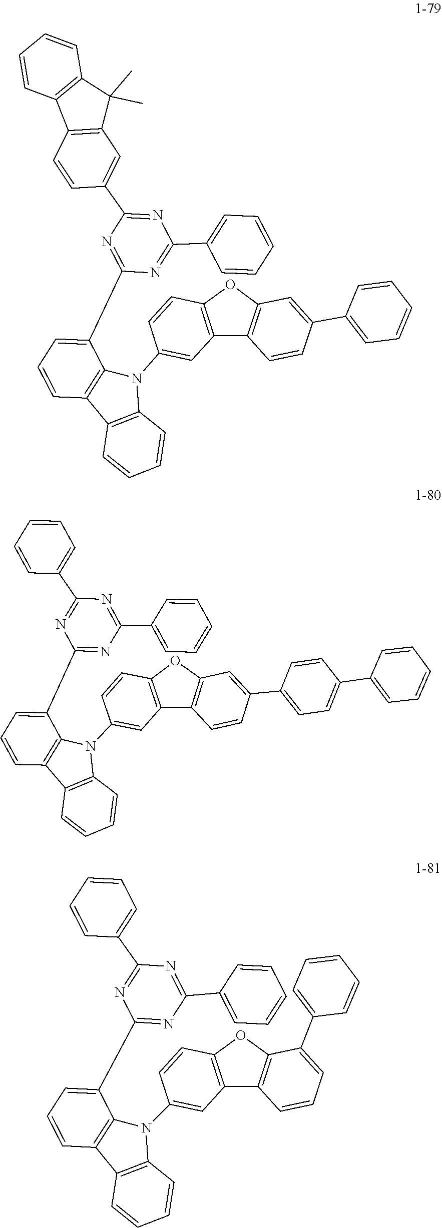

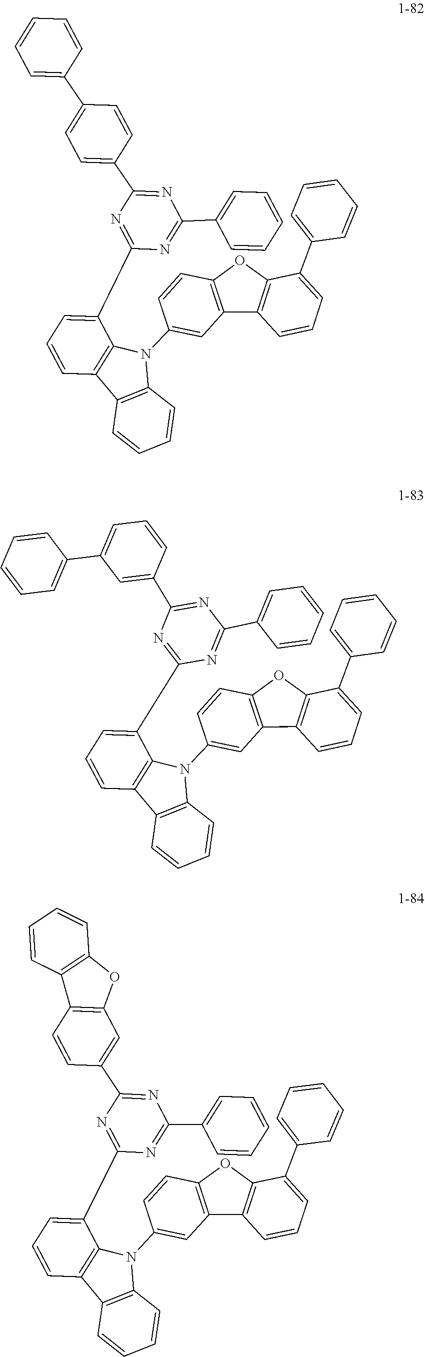 [SMILES: c1ccc(-c2ccc(-c3nc(-c4ccccc4)nc(-c4cccc5c6ccccc6n(-c6ccc7oc8c(-c9ccccc9)cccc8c7c6)c45)n3)cc2)cc1 ; c1ccc(-c2cccc(-c3nc(-c4ccccc4)nc(-c4cccc5c6ccccc6n(-c6ccc7oc8c(-c9ccccc9)cccc8c7c6)c45)n3)c2)cc1 ; c1ccc(-c2nc(-c3ccc4c(c3)oc3ccccc34)nc(-c3cccc4c5ccccc5n(-c5ccc6oc7c(-c8ccccc8)cccc7c6c5)c34)n2)cc1]

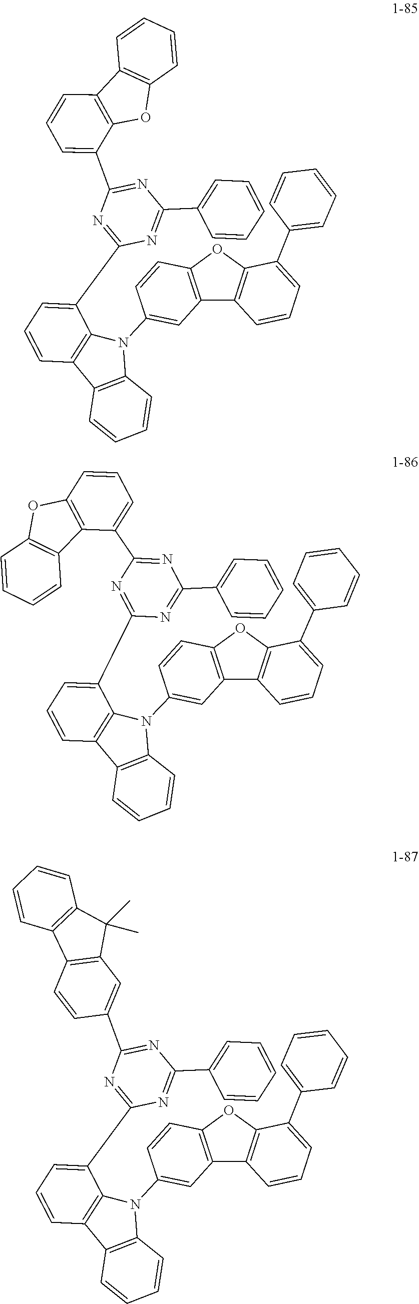

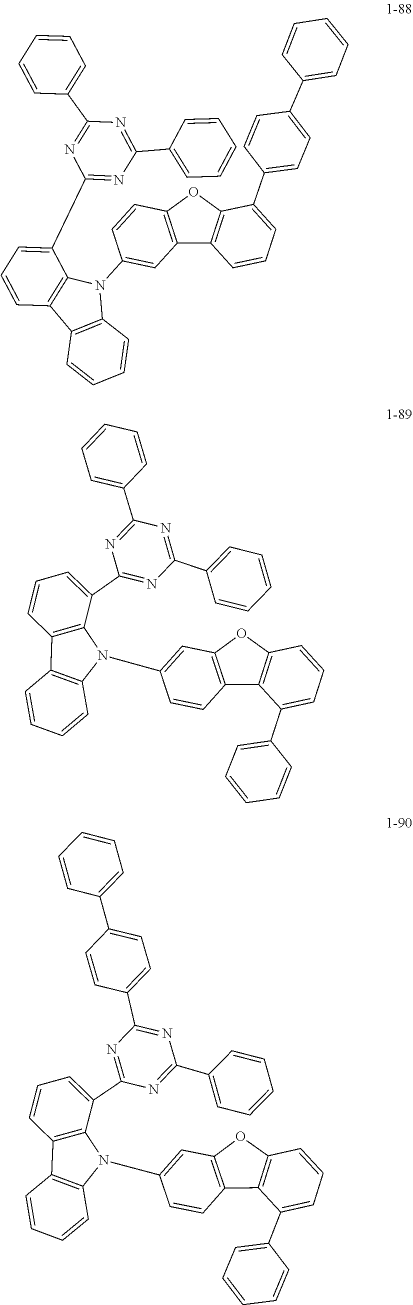

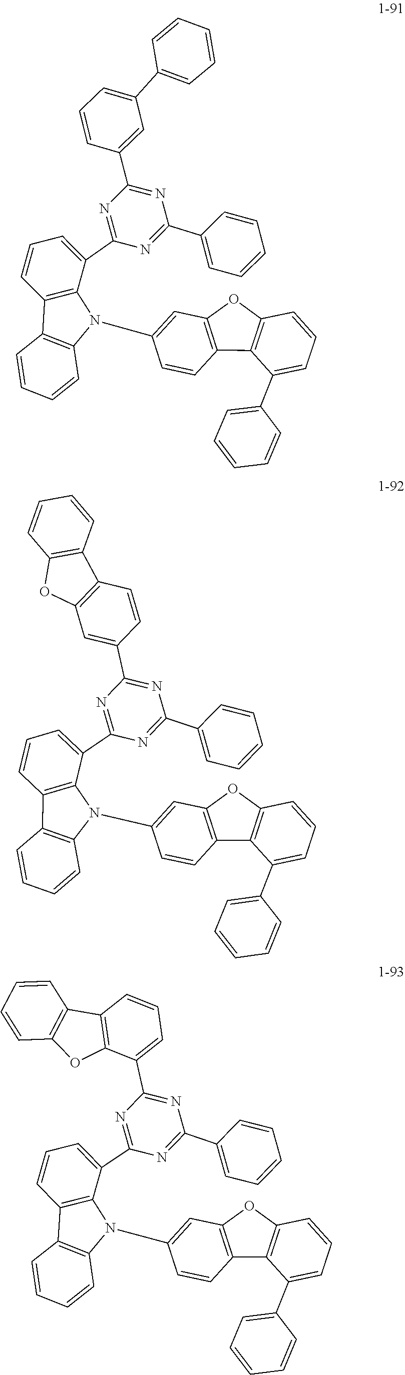 [SMILES: c1ccc(-c2cccc(-c3nc(-c4ccccc4)nc(-c4cccc5c6ccccc6n(-c6ccc7c(c6)oc6cccc(-c8ccccc8)c67)c45)n3)c2)cc1 ; c1ccc(-c2nc(-c3ccc4c(c3)oc3ccccc34)nc(-c3cccc4c5ccccc5n(-c5ccc6c(c5)oc5cccc(-c7ccccc7)c56)c34)n2)cc1 ; c1ccc(-c2nc(-c3cccc4c3oc3ccccc34)nc(-c3cccc4c5ccccc5n(-c5ccc6c(c5)oc5cccc(-c7ccccc7)c56)c34)n2)cc1]

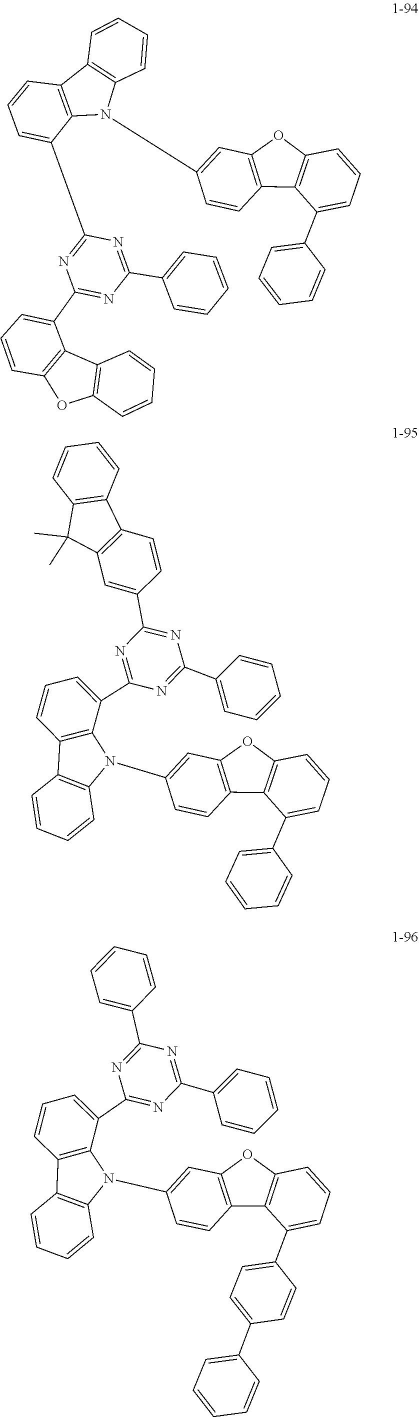

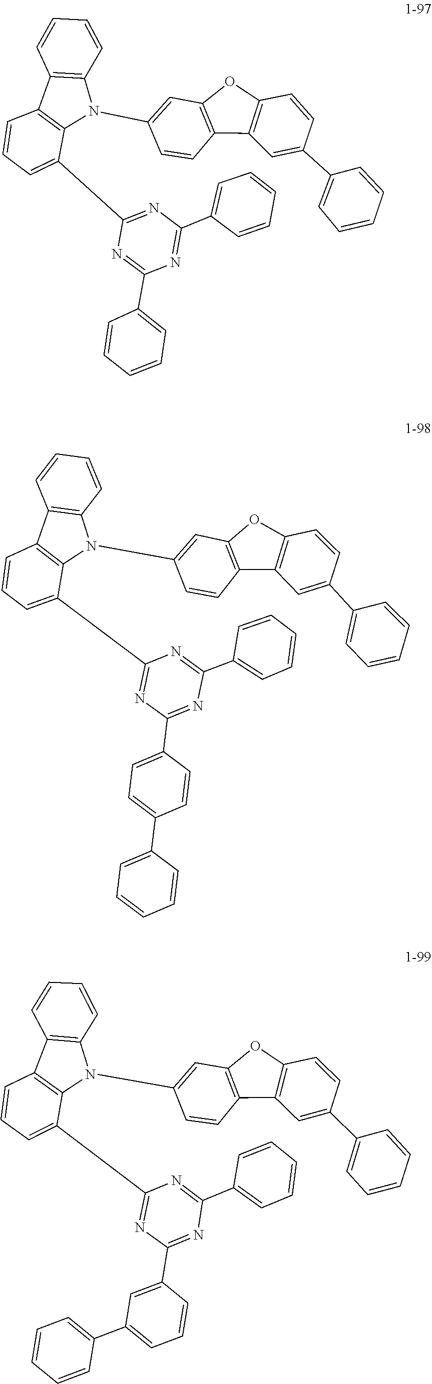 [SMILES: c1ccc(-c2ccc3oc4cc(-n5c6ccccc6c6cccc(-c7nc(-c8ccccc8)nc(-c8ccccc8)n7)c65)ccc4c3c2)cc1 ; c1ccc(-c2ccc(-c3nc(-c4ccccc4)nc(-c4cccc5c6ccccc6n(-c6ccc7c(c6)oc6ccc(-c8ccccc8)cc67)c45)n3)cc2)cc1 ; c1ccc(-c2cccc(-c3nc(-c4ccccc4)nc(-c4cccc5c6ccccc6n(-c6ccc7c(c6)oc6ccc(-c8ccccc8)cc67)c45)n3)c2)cc1]

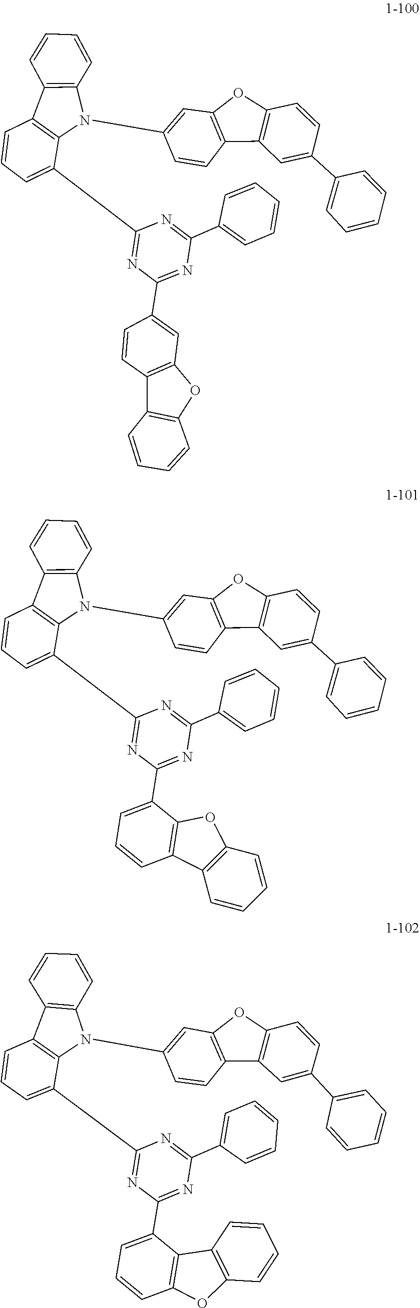 [SMILES: c1ccc(-c2ccc3oc4cc(-n5c6ccccc6c6cccc(-c7nc(-c8ccccc8)nc(-c8ccc9c(c8)oc8ccccc89)n7)c65)ccc4c3c2)cc1 ; c1ccc(-c2ccc3oc4cc(-n5c6ccccc6c6cccc(-c7nc(-c8ccccc8)nc(-c8cccc9c8oc8ccccc89)n7)c65)ccc4c3c2)cc1 ; c1ccc(-c2ccc3oc4cc(-n5c6ccccc6c6cccc(-c7nc(-c8ccccc8)nc(-c8cccc9oc%10ccccc%10c89)n7)c65)ccc4c3c2)cc1]

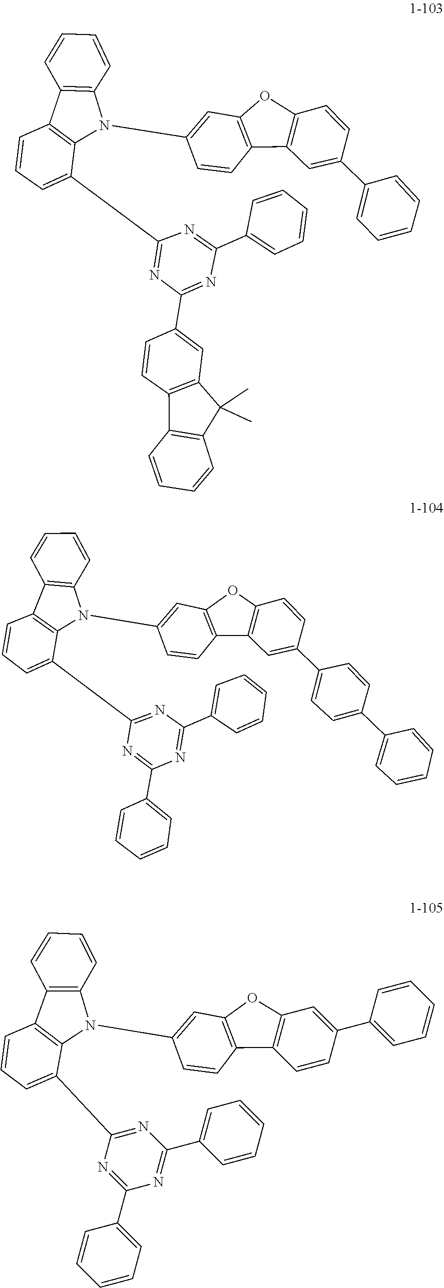 [SMILES: CC1(C)c2ccccc2-c2ccc(-c3nc(-c4ccccc4)nc(-c4cccc5c6ccccc6n(-c6ccc7c(c6)oc6ccc(-c8ccccc8)cc67)c45)n3)cc21 ; c1ccc(-c2ccc(-c3ccc4oc5cc(-n6c7ccccc7c7cccc(-c8nc(-c9ccccc9)nc(-c9ccccc9)n8)c76)ccc5c4c3)cc2)cc1 ; c1ccc(-c2ccc3c(c2)oc2cc(-n4c5ccccc5c5cccc(-c6nc(-c7ccccc7)nc(-c7ccccc7)n6)c54)ccc23)cc1]

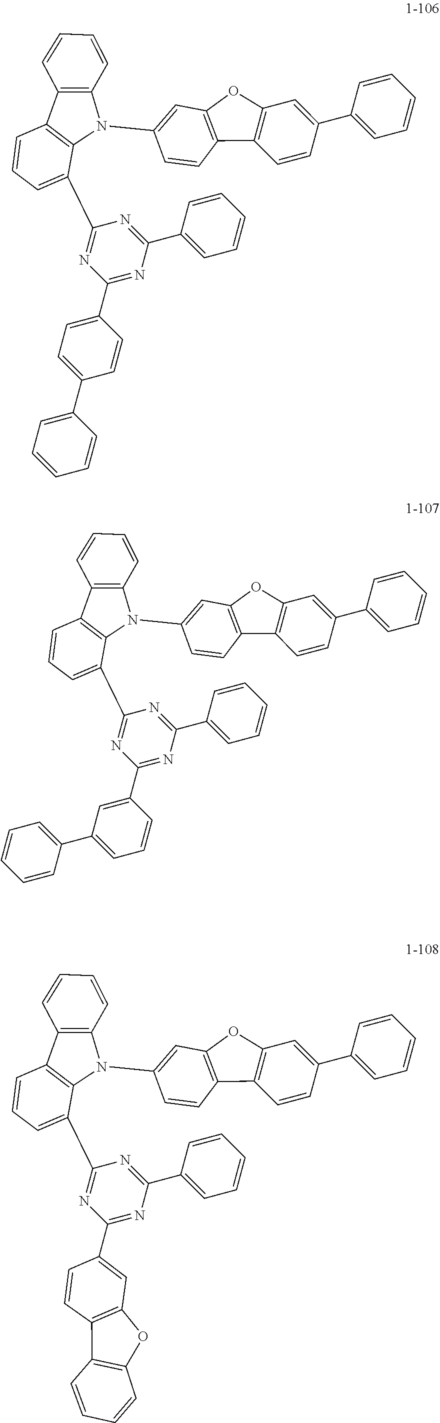 [SMILES: c1ccc(-c2ccc(-c3nc(-c4ccccc4)nc(-c4cccc5c6ccccc6n(-c6ccc7c(c6)oc6cc(-c8ccccc8)ccc67)c45)n3)cc2)cc1 ; c1ccc(-c2cccc(-c3nc(-c4ccccc4)nc(-c4cccc5c6ccccc6n(-c6ccc7c(c6)oc6cc(-c8ccccc8)ccc67)c45)n3)c2)cc1 ; c1ccc(-c2ccc3c(c2)oc2cc(-n4c5ccccc5c5cccc(-c6nc(-c7ccccc7)nc(-c7ccc8c(c7)oc7ccccc78)n6)c54)ccc23)cc1]

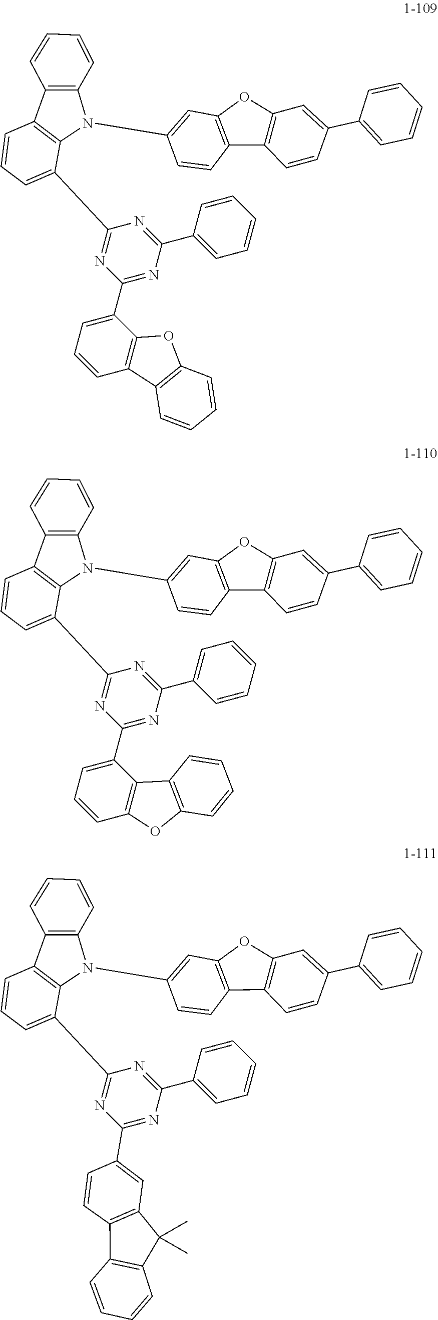 [SMILES: c1ccc(-c2ccc3c(c2)oc2cc(-n4c5ccccc5c5cccc(-c6nc(-c7ccccc7)nc(-c7cccc8c7oc7ccccc78)n6)c54)ccc23)cc1 ; c1ccc(-c2ccc3c(c2)oc2cc(-n4c5ccccc5c5cccc(-c6nc(-c7ccccc7)nc(-c7cccc8oc9ccccc9c78)n6)c54)ccc23)cc1 ; CC1(C)c2ccccc2-c2ccc(-c3nc(-c4ccccc4)nc(-c4cccc5c6ccccc6n(-c6ccc7c(c6)oc6cc(-c8ccccc8)ccc67)c45)n3)cc21]

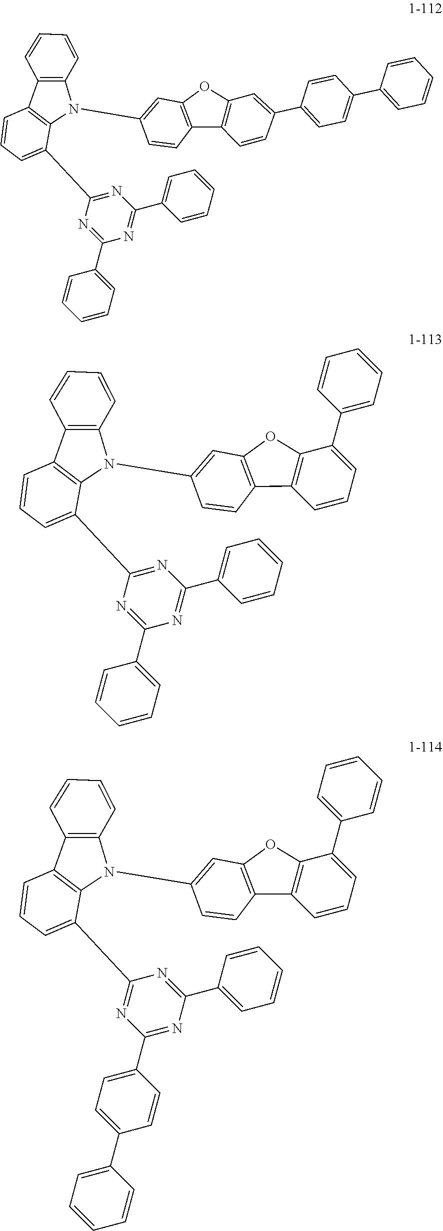 [SMILES: c1ccc(-c2ccc(-c3ccc4c(c3)oc3cc(-n5c6ccccc6c6cccc(-c7nc(-c8ccccc8)nc(-c8ccccc8)n7)c65)ccc34)cc2)cc1 ; c1ccc(-c2nc(-c3ccccc3)nc(-c3cccc4c5ccccc5n(-c5ccc6c(c5)oc5c(-c7ccccc7)cccc56)c34)n2)cc1 ; c1ccc(-c2ccc(-c3nc(-c4ccccc4)nc(-c4cccc5c6ccccc6n(-c6ccc7c(c6)oc6c(-c8ccccc8)cccc67)c45)n3)cc2)cc1]

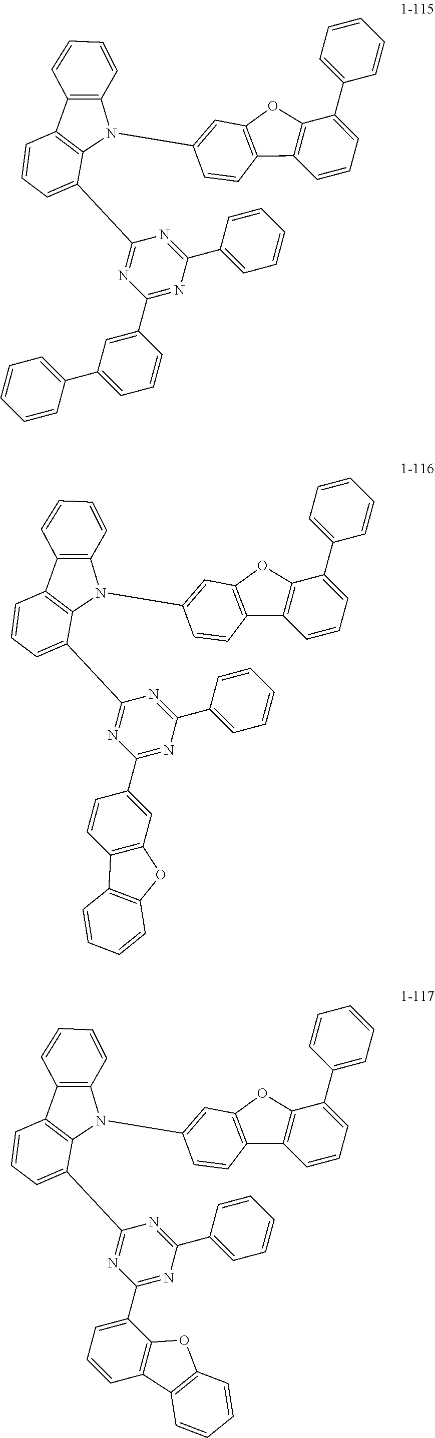 [SMILES: c1ccc(-c2cccc(-c3nc(-c4ccccc4)nc(-c4cccc5c6ccccc6n(-c6ccc7c(c6)oc6c(-c8ccccc8)cccc67)c45)n3)c2)cc1 ; c1ccc(-c2nc(-c3ccc4c(c3)oc3ccccc34)nc(-c3cccc4c5ccccc5n(-c5ccc6c(c5)oc5c(-c7ccccc7)cccc56)c34)n2)cc1 ; c1ccc(-c2nc(-c3cccc4c3oc3ccccc34)nc(-c3cccc4c5ccccc5n(-c5ccc6c(c5)oc5c(-c7ccccc7)cccc56)c34)n2)cc1]

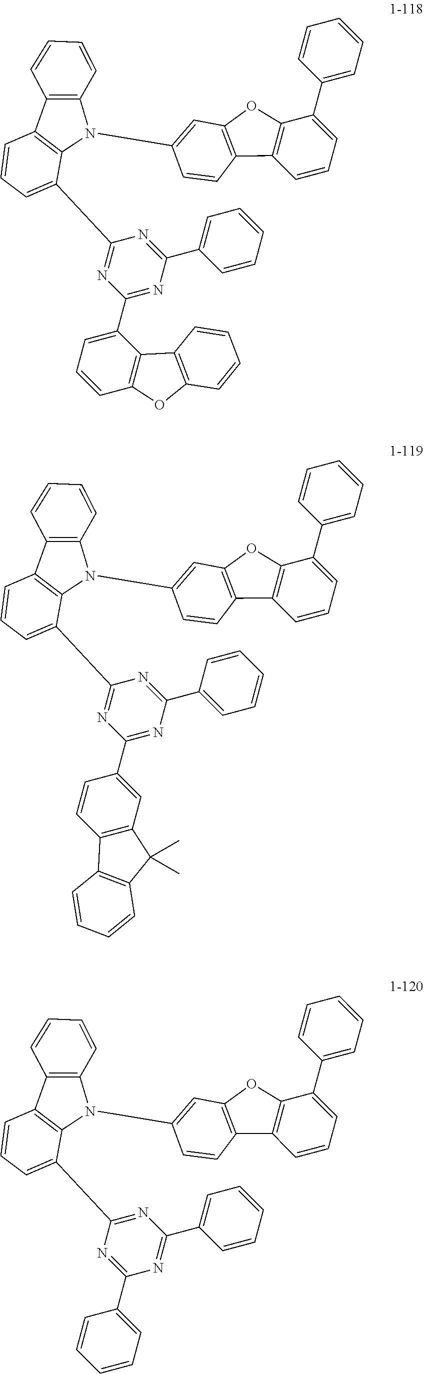 [SMILES: c1ccc(-c2nc(-c3cccc4oc5ccccc5c34)nc(-c3cccc4c5ccccc5n(-c5ccc6c(c5)oc5c(-c7ccccc7)cccc56)c34)n2)cc1 ; CC1(C)c2ccccc2-c2ccc(-c3nc(-c4ccccc4)nc(-c4cccc5c6ccccc6n(-c6ccc7c(c6)oc6c(-c8ccccc8)cccc67)c45)n3)cc21 ; c1ccc(-c2nc(-c3ccccc3)nc(-c3cccc4c5ccccc5n(-c5ccc6c(c5)oc5c(-c7ccccc7)cccc56)c34)n2)cc1]

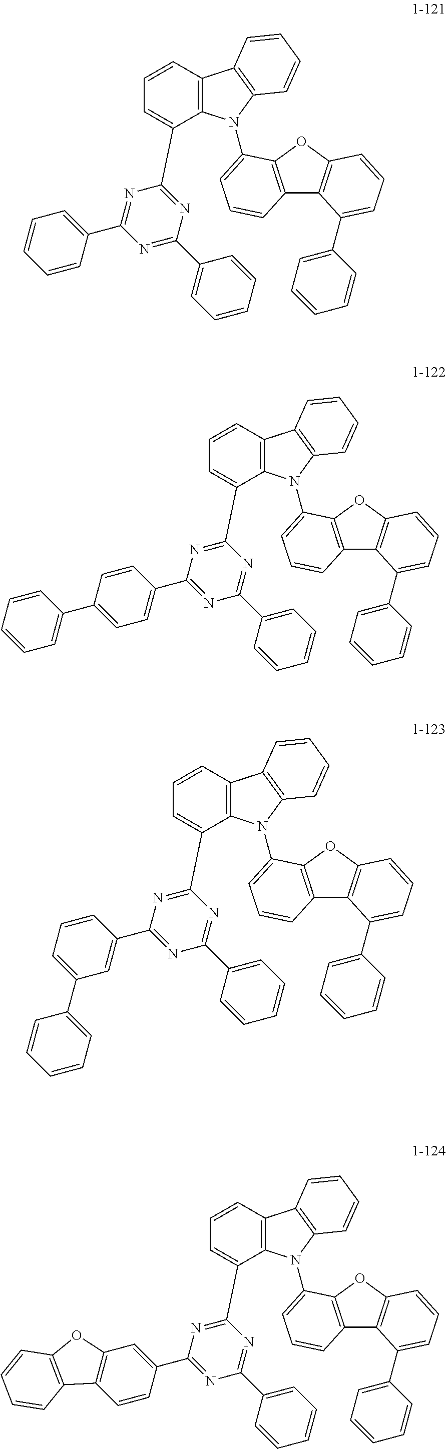 [SMILES: c1ccc(-c2nc(-c3ccccc3)nc(-c3cccc4c5ccccc5n(-c5cccc6c5oc5cccc(-c7ccccc7)c56)c34)n2)cc1 ; c1ccc(-c2ccc(-c3nc(-c4ccccc4)nc(-c4cccc5c6ccccc6n(-c6cccc7c6oc6cccc(-c8ccccc8)c67)c45)n3)cc2)cc1 ; c1ccc(-c2cccc(-c3nc(-c4ccccc4)nc(-c4cccc5c6ccccc6n(-c6cccc7c6oc6cccc(-c8ccccc8)c67)c45)n3)c2)cc1 ; c1ccc(-c2nc(-c3ccc4c(c3)oc3ccccc34)nc(-c3cccc4c5ccccc5n(-c5cccc6c5oc5cccc(-c7ccccc7)c56)c34)n2)cc1]

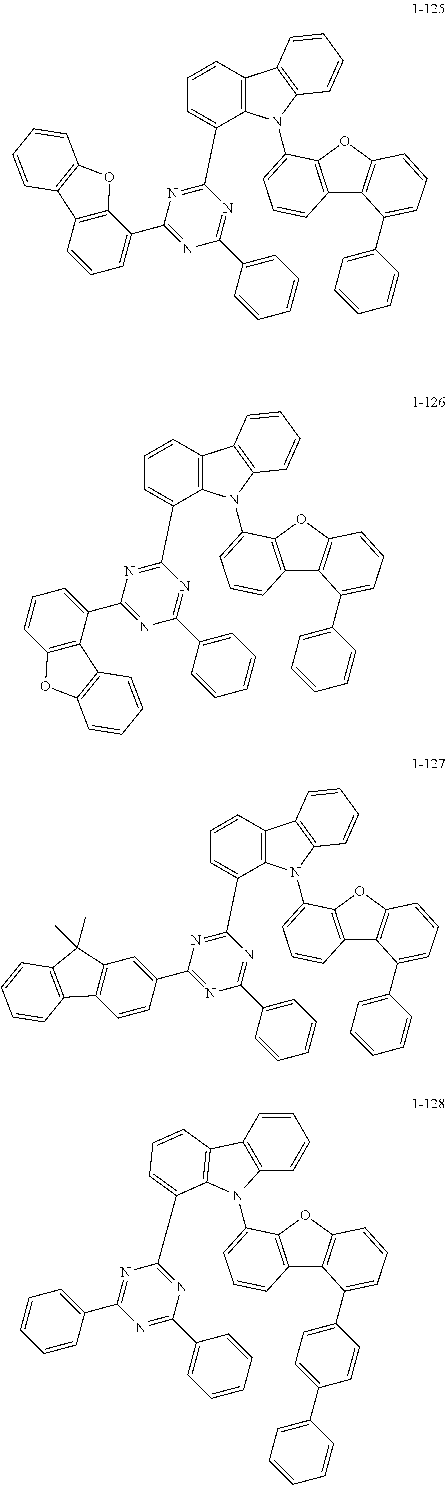 [SMILES: c1ccc(-c2nc(-c3cccc4c3oc3ccccc34)nc(-c3cccc4c5ccccc5n(-c5cccc6c5oc5cccc(-c7ccccc7)c56)c34)n2)cc1 ; c1ccc(-c2nc(-c3cccc4oc5ccccc5c34)nc(-c3cccc4c5ccccc5n(-c5cccc6c5oc5cccc(-c7ccccc7)c56)c34)n2)cc1 ; CC1(C)c2ccccc2-c2ccc(-c3nc(-c4ccccc4)nc(-c4cccc5c6ccccc6n(-c6cccc7c6oc6cccc(-c8ccccc8)c67)c45)n3)cc21 ; c1ccc(-c2ccc(-c3cccc4oc5c(-n6c7ccccc7c7cccc(-c8nc(-c9ccccc9)nc(-c9ccccc9)n8)c76)cccc5c34)cc2)cc1]

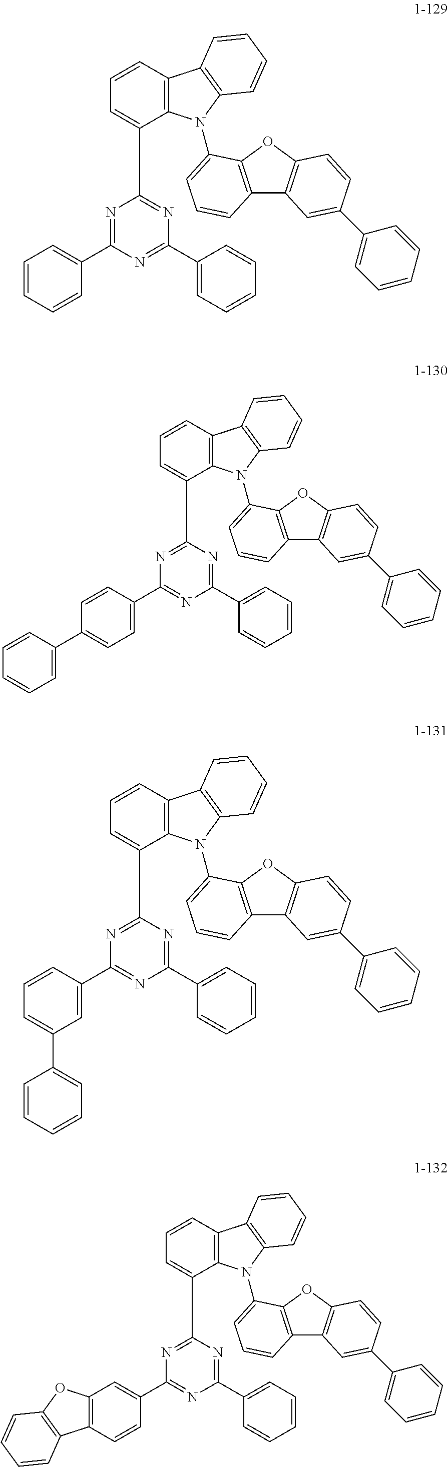 [SMILES: c1ccc(-c2ccc3oc4c(-n5c6ccccc6c6cccc(-c7nc(-c8ccccc8)nc(-c8ccccc8)n7)c65)cccc4c3c2)cc1 ; c1ccc(-c2ccc(-c3nc(-c4ccccc4)nc(-c4cccc5c6ccccc6n(-c6cccc7c6oc6ccc(-c8ccccc8)cc67)c45)n3)cc2)cc1 ; c1ccc(-c2cccc(-c3nc(-c4ccccc4)nc(-c4cccc5c6ccccc6n(-c6cccc7c6oc6ccc(-c8ccccc8)cc67)c45)n3)c2)cc1 ; c1ccc(-c2ccc3oc4c(-n5c6ccccc6c6cccc(-c7nc(-c8ccccc8)nc(-c8ccc9c(c8)oc8ccccc89)n7)c65)cccc4c3c2)cc1]

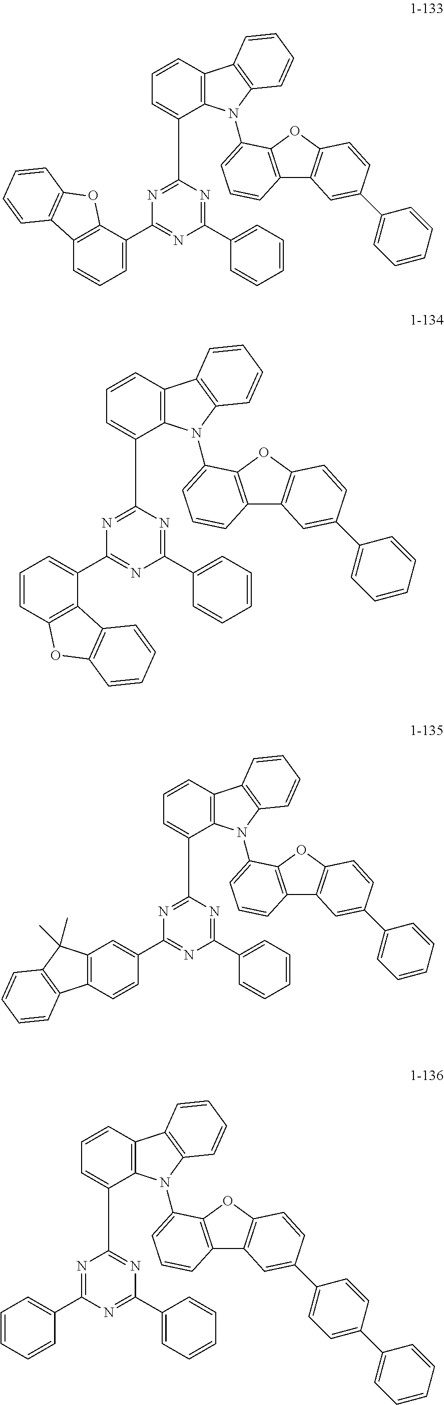 [SMILES: c1ccc(-c2ccc3oc4c(-n5c6ccccc6c6cccc(-c7nc(-c8ccccc8)nc(-c8cccc9c8oc8ccccc89)n7)c65)cccc4c3c2)cc1 ; c1ccc(-c2ccc3oc4c(-n5c6ccccc6c6cccc(-c7nc(-c8ccccc8)nc(-c8cccc9oc%10ccccc%10c89)n7)c65)cccc4c3c2)cc1 ; CC1(C)c2ccccc2-c2ccc(-c3nc(-c4ccccc4)nc(-c4cccc5c6ccccc6n(-c6cccc7c6oc6ccc(-c8ccccc8)cc67)c45)n3)cc21 ; c1ccc(-c2ccc(-c3ccc4oc5c(-n6c7ccccc7c7cccc(-c8nc(-c9ccccc9)nc(-c9ccccc9)n8)c76)cccc5c4c3)cc2)cc1]

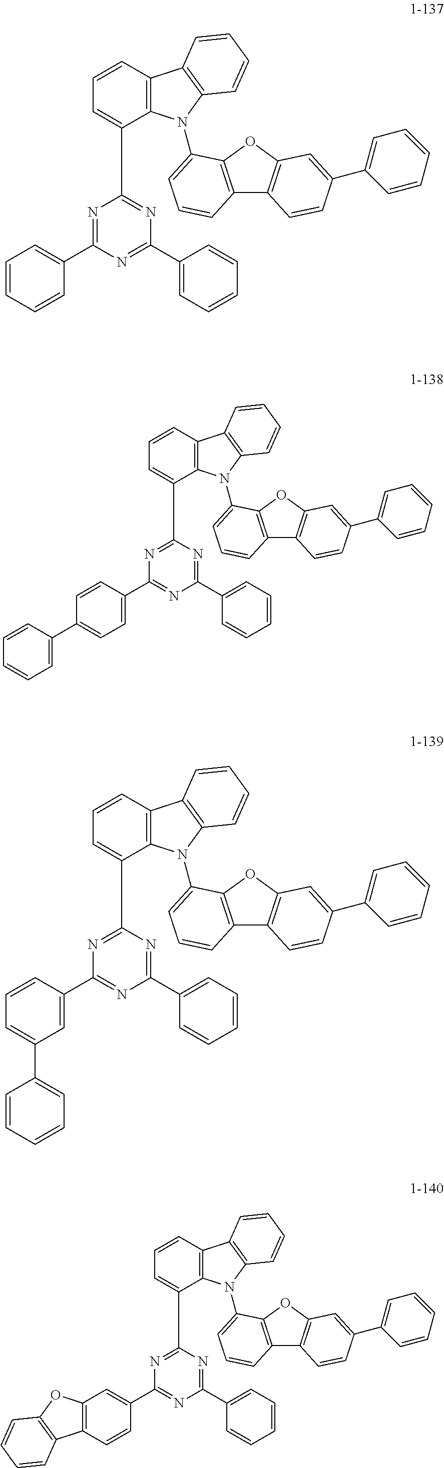 [SMILES: c1ccc(-c2ccc3c(c2)oc2c(-n4c5ccccc5c5cccc(-c6nc(-c7ccccc7)nc(-c7ccccc7)n6)c54)cccc23)cc1 ; c1ccc(-c2ccc(-c3nc(-c4ccccc4)nc(-c4cccc5c6ccccc6n(-c6cccc7c6oc6cc(-c8ccccc8)ccc67)c45)n3)cc2)cc1 ; c1ccc(-c2cccc(-c3nc(-c4ccccc4)nc(-c4cccc5c6ccccc6n(-c6cccc7c6oc6cc(-c8ccccc8)ccc67)c45)n3)c2)cc1 ; c1ccc(-c2ccc3c(c2)oc2c(-n4c5ccccc5c5cccc(-c6nc(-c7ccccc7)nc(-c7ccc8c(c7)oc7ccccc78)n6)c54)cccc23)cc1]

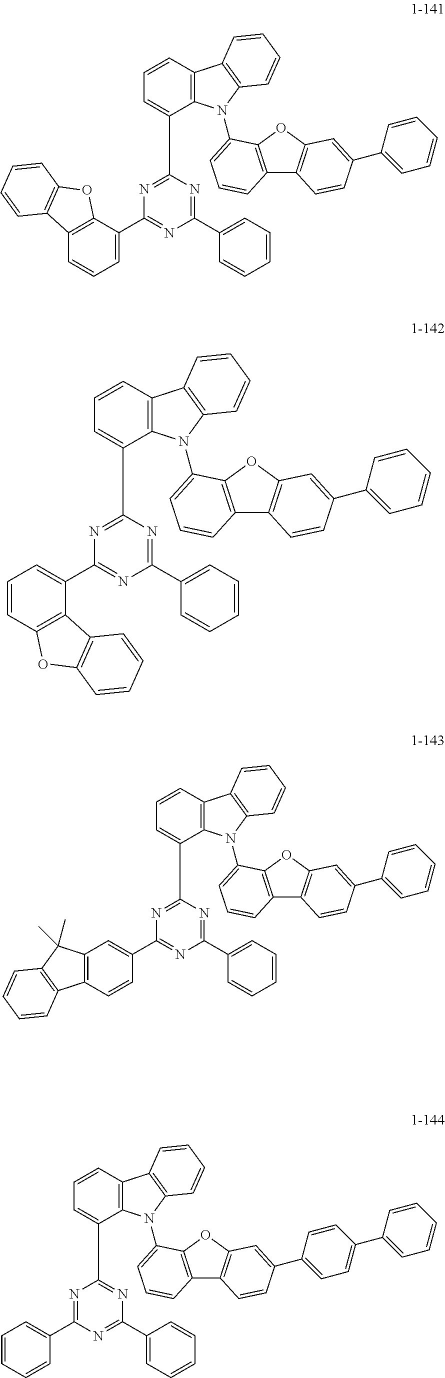 [SMILES: c1ccc(-c2ccc3c(c2)oc2c(-n4c5ccccc5c5cccc(-c6nc(-c7ccccc7)nc(-c7cccc8c7oc7ccccc78)n6)c54)cccc23)cc1 ; c1ccc(-c2ccc3c(c2)oc2c(-n4c5ccccc5c5cccc(-c6nc(-c7ccccc7)nc(-c7cccc8oc9ccccc9c78)n6)c54)cccc23)cc1 ; CC1(C)c2ccccc2-c2ccc(-c3nc(-c4ccccc4)nc(-c4cccc5c6ccccc6n(-c6cccc7c6oc6cc(-c8ccccc8)ccc67)c45)n3)cc21 ; c1ccc(-c2ccc(-c3ccc4c(c3)oc3c(-n5c6ccccc6c6cccc(-c7nc(-c8ccccc8)nc(-c8ccccc8)n7)c65)cccc34)cc2)cc1]

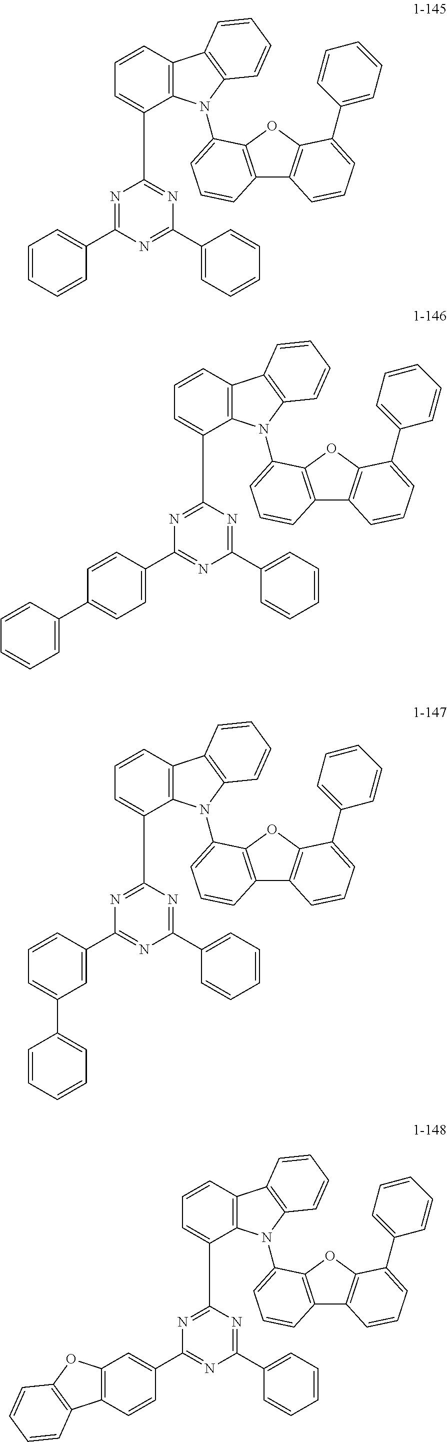 [SMILES: c1ccc(-c2nc(-c3ccccc3)nc(-c3cccc4c5ccccc5n(-c5cccc6c5oc5c(-c7ccccc7)cccc56)c34)n2)cc1 ; c1ccc(-c2ccc(-c3nc(-c4ccccc4)nc(-c4cccc5c6ccccc6n(-c6cccc7c6oc6c(-c8ccccc8)cccc67)c45)n3)cc2)cc1 ; c1ccc(-c2cccc(-c3nc(-c4ccccc4)nc(-c4cccc5c6ccccc6n(-c6cccc7c6oc6c(-c8ccccc8)cccc67)c45)n3)c2)cc1 ; c1ccc(-c2nc(-c3ccc4c(c3)oc3ccccc34)nc(-c3cccc4c5ccccc5n(-c5cccc6c5oc5c(-c7ccccc7)cccc56)c34)n2)cc1]

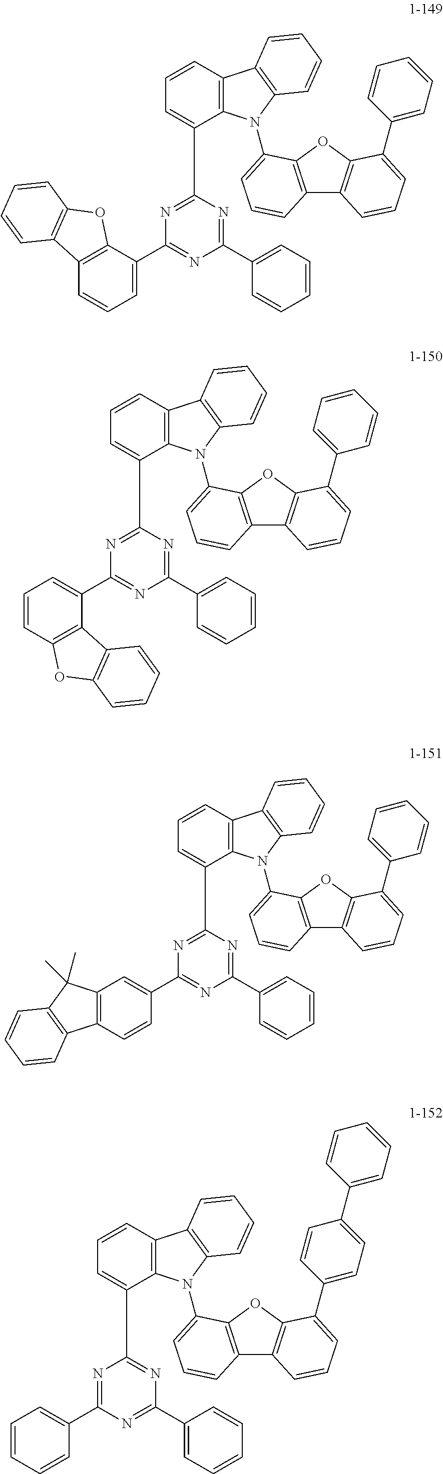 [SMILES: c1ccc(-c2nc(-c3cccc4c3oc3ccccc34)nc(-c3cccc4c5ccccc5n(-c5cccc6c5oc5c(-c7ccccc7)cccc56)c34)n2)cc1 ; c1ccc(-c2nc(-c3cccc4oc5ccccc5c34)nc(-c3cccc4c5ccccc5n(-c5cccc6c5oc5c(-c7ccccc7)cccc56)c34)n2)cc1 ; CC1(C)c2ccccc2-c2ccc(-c3nc(-c4ccccc4)nc(-c4cccc5c6ccccc6n(-c6cccc7c6oc6c(-c8ccccc8)cccc67)c45)n3)cc21 ; c1ccc(-c2ccc(-c3cccc4c3oc3c(-n5c6ccccc6c6cccc(-c7nc(-c8ccccc8)nc(-c8ccccc8)n7)c65)cccc34)cc2)cc1]

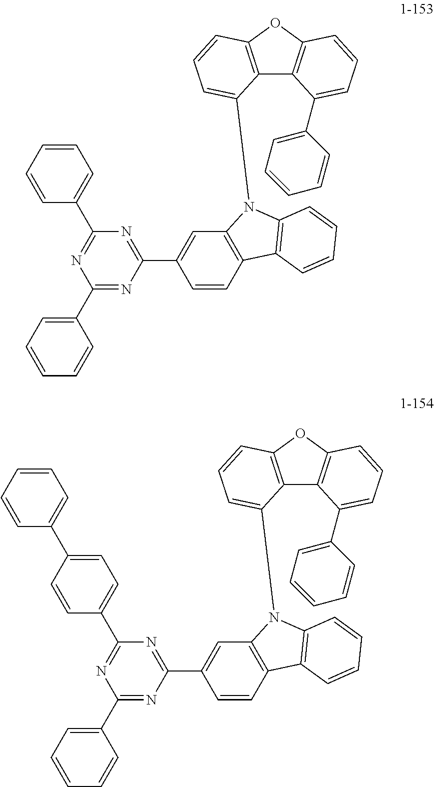 [SMILES: c1ccc(-c2nc(-c3ccccc3)nc(-c3ccc4c5ccccc5n(-c5cccc6oc7cccc(-c8ccccc8)c7c56)c4c3)n2)cc1 ; c1ccc(-c2ccc(-c3nc(-c4ccccc4)nc(-c4ccc5c6ccccc6n(-c6cccc7oc8cccc(-c9ccccc9)c8c67)c5c4)n3)cc2)cc1]

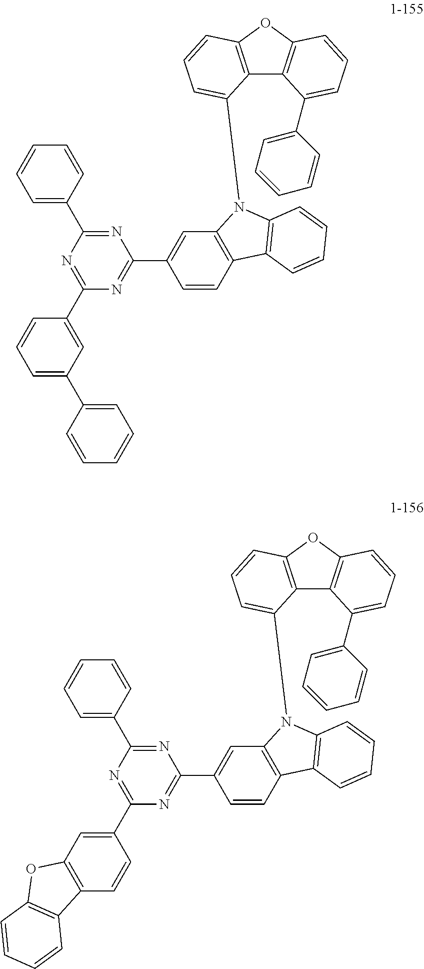 [SMILES: c1ccc(-c2cccc(-c3nc(-c4ccccc4)nc(-c4ccc5c6ccccc6n(-c6cccc7oc8cccc(-c9ccccc9)c8c67)c5c4)n3)c2)cc1 ; c1ccc(-c2nc(-c3ccc4c(c3)oc3ccccc34)nc(-c3ccc4c5ccccc5n(-c5cccc6oc7cccc(-c8ccccc8)c7c56)c4c3)n2)cc1]

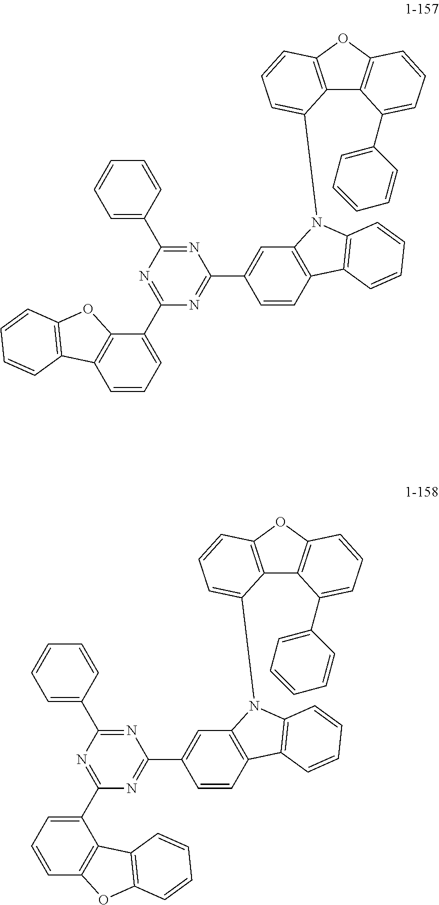 [SMILES: c1ccc(-c2nc(-c3ccc4c5ccccc5n(-c5cccc6oc7cccc(-c8ccccc8)c7c56)c4c3)nc(-c3cccc4c3oc3ccccc34)n2)cc1 ; c1ccc(-c2nc(-c3ccc4c5ccccc5n(-c5cccc6oc7cccc(-c8ccccc8)c7c56)c4c3)nc(-c3cccc4oc5ccccc5c34)n2)cc1]

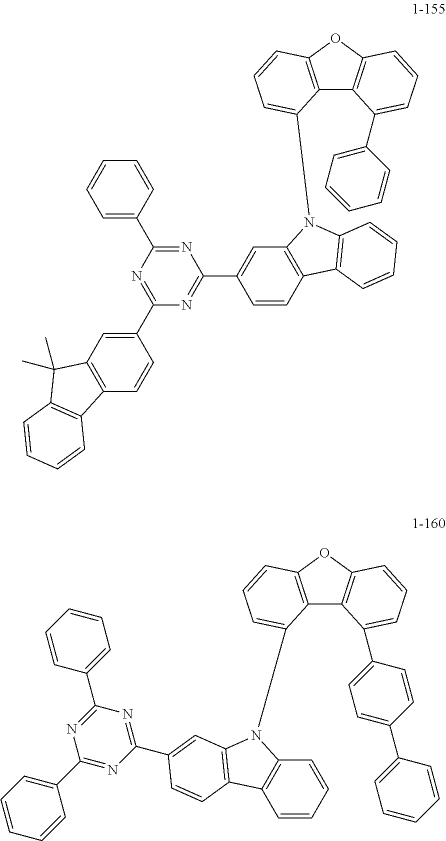 [SMILES: CC1(C)c2ccccc2-c2ccc(-c3nc(-c4ccccc4)nc(-c4ccc5c6ccccc6n(-c6cccc7oc8cccc(-c9ccccc9)c8c67)c5c4)n3)cc21 ; c1ccc(-c2ccc(-c3cccc4oc5cccc(-n6c7ccccc7c7ccc(-c8nc(-c9ccccc9)nc(-c9ccccc9)n8)cc76)c5c34)cc2)cc1]

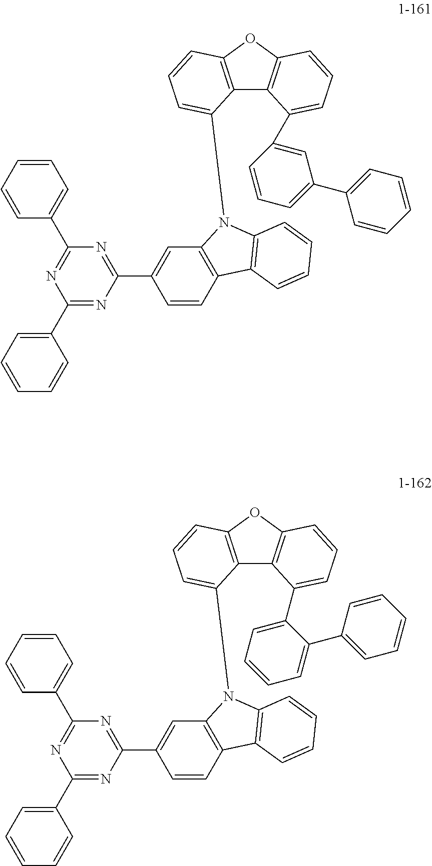 [SMILES: c1ccc(-c2cccc(-c3cccc4oc5cccc(-n6c7ccccc7c7ccc(-c8nc(-c9ccccc9)nc(-c9ccccc9)n8)cc76)c5c34)c2)cc1 ; c1ccc(-c2nc(-c3ccccc3)nc(-c3ccc4c5ccccc5n(-c5cccc6oc7cccc(-c8ccccc8-c8ccccc8)c7c56)c4c3)n2)cc1]

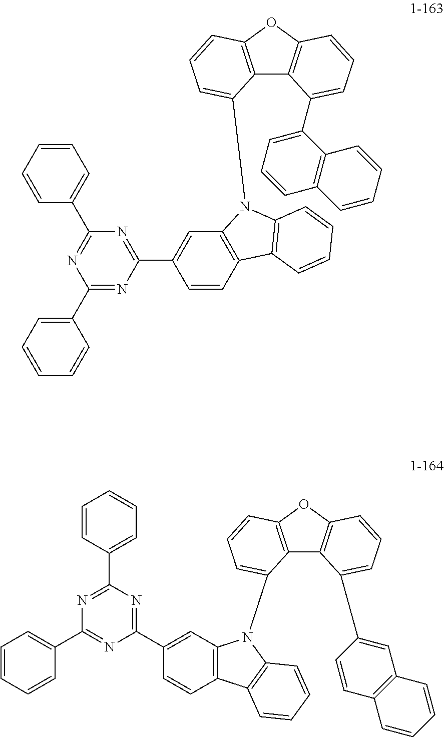 [SMILES: c1ccc(-c2nc(-c3ccccc3)nc(-c3ccc4c5ccccc5n(-c5cccc6oc7cccc(-c8cccc9ccccc89)c7c56)c4c3)n2)cc1 ; c1ccc(-c2nc(-c3ccccc3)nc(-c3ccc4c5ccccc5n(-c5cccc6oc7cccc(-c8ccc9ccccc9c8)c7c56)c4c3)n2)cc1]

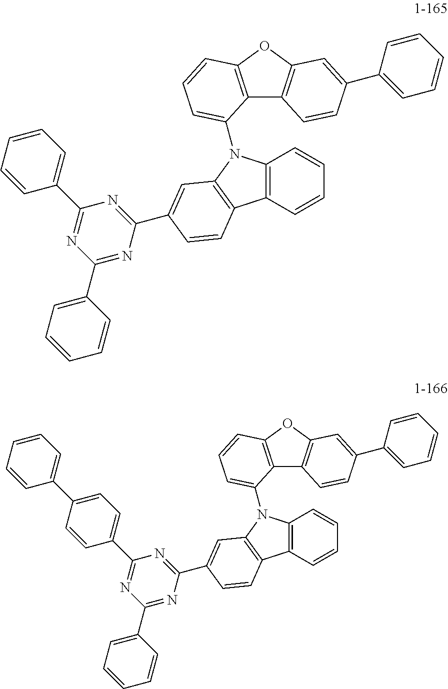 [SMILES: c1ccc(-c2ccc3c(c2)oc2cccc(-n4c5ccccc5c5ccc(-c6nc(-c7ccccc7)nc(-c7ccccc7)n6)cc54)c23)cc1 ; c1ccc(-c2ccc(-c3nc(-c4ccccc4)nc(-c4ccc5c6ccccc6n(-c6cccc7oc8cc(-c9ccccc9)ccc8c67)c5c4)n3)cc2)cc1]

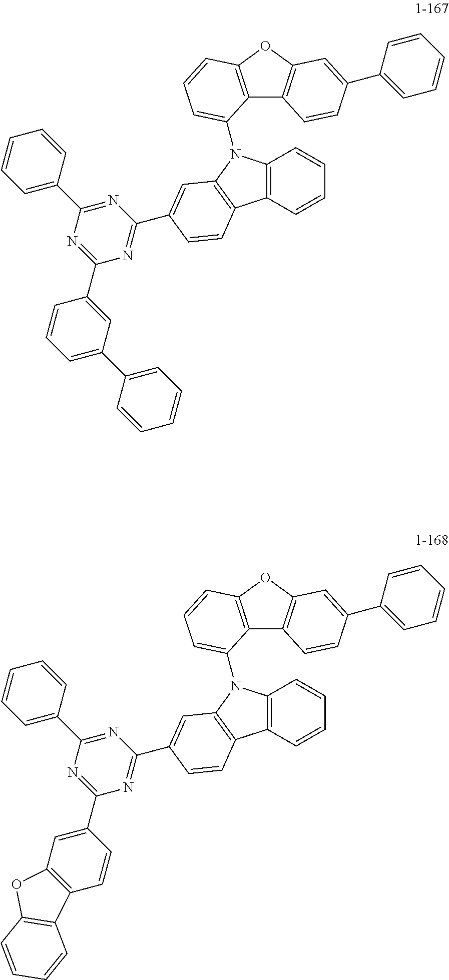 [SMILES: c1ccc(-c2cccc(-c3nc(-c4ccccc4)nc(-c4ccc5c6ccccc6n(-c6cccc7oc8cc(-c9ccccc9)ccc8c67)c5c4)n3)c2)cc1 ; c1ccc(-c2ccc3c(c2)oc2cccc(-n4c5ccccc5c5ccc(-c6nc(-c7ccccc7)nc(-c7ccc8c(c7)oc7ccccc78)n6)cc54)c23)cc1]

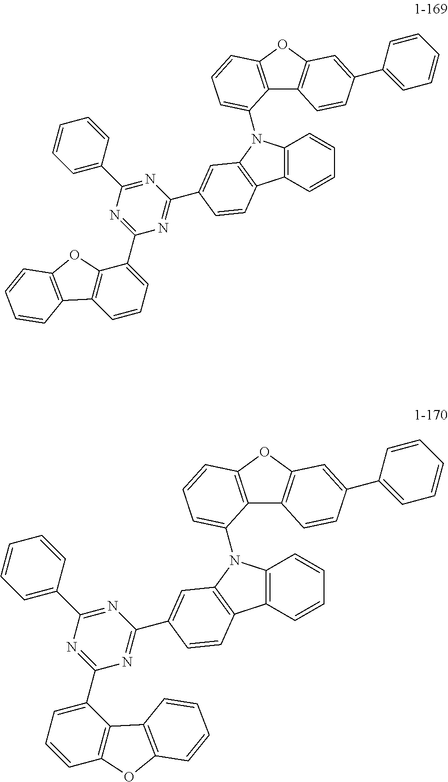 [SMILES: c1ccc(-c2ccc3c(c2)oc2cccc(-n4c5ccccc5c5ccc(-c6nc(-c7ccccc7)nc(-c7cccc8c7oc7ccccc78)n6)cc54)c23)cc1 ; c1ccc(-c2ccc3c(c2)oc2cccc(-n4c5ccccc5c5ccc(-c6nc(-c7ccccc7)nc(-c7cccc8oc9ccccc9c78)n6)cc54)c23)cc1]

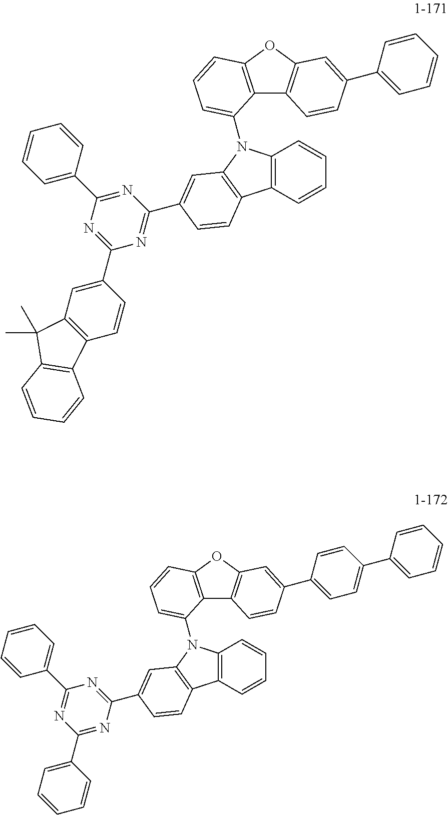 [SMILES: CC1(C)c2ccccc2-c2ccc(-c3nc(-c4ccccc4)nc(-c4ccc5c6ccccc6n(-c6cccc7oc8cc(-c9ccccc9)ccc8c67)c5c4)n3)cc21 ; c1ccc(-c2ccc(-c3ccc4c(c3)oc3cccc(-n5c6ccccc6c6ccc(-c7nc(-c8ccccc8)nc(-c8ccccc8)n7)cc65)c34)cc2)cc1]

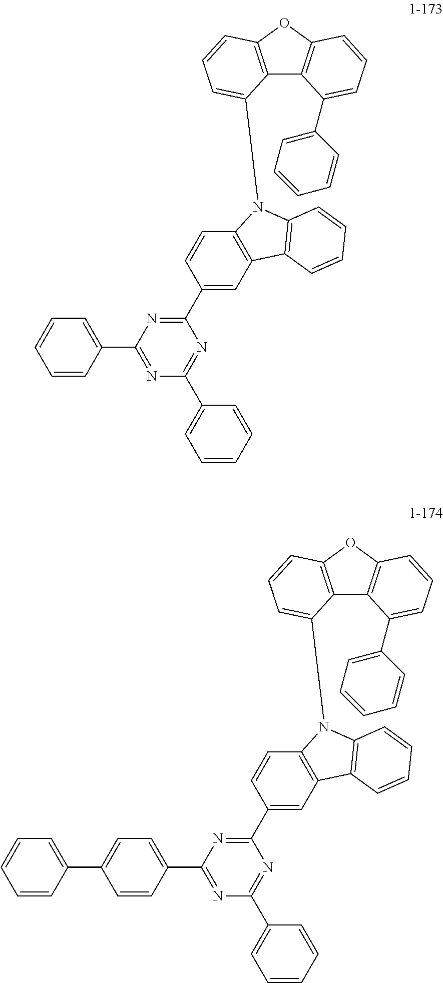 [SMILES: c1ccc(-c2nc(-c3ccccc3)nc(-c3ccc4c(c3)c3ccccc3n4-c3cccc4oc5cccc(-c6ccccc6)c5c34)n2)cc1 ; c1ccc(-c2ccc(-c3nc(-c4ccccc4)nc(-c4ccc5c(c4)c4ccccc4n5-c4cccc5oc6cccc(-c7ccccc7)c6c45)n3)cc2)cc1]

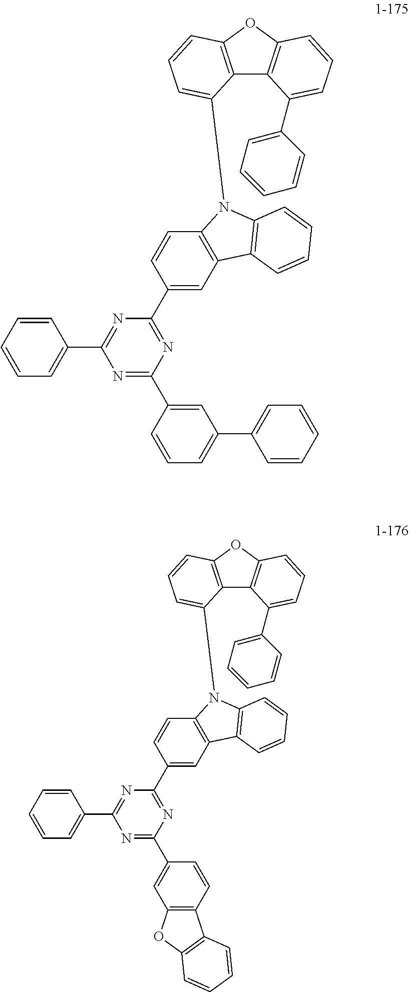 [SMILES: c1ccc(-c2cccc(-c3nc(-c4ccccc4)nc(-c4ccc5c(c4)c4ccccc4n5-c4cccc5oc6cccc(-c7ccccc7)c6c45)n3)c2)cc1 ; c1ccc(-c2nc(-c3ccc4c(c3)oc3ccccc34)nc(-c3ccc4c(c3)c3ccccc3n4-c3cccc4oc5cccc(-c6ccccc6)c5c34)n2)cc1]

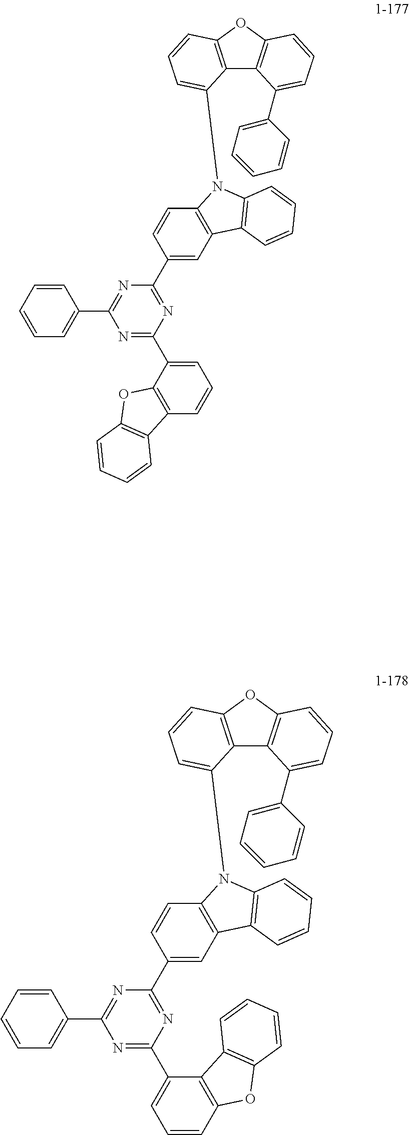 [SMILES: c1ccc(-c2nc(-c3ccc4c(c3)c3ccccc3n4-c3cccc4oc5cccc(-c6ccccc6)c5c34)nc(-c3cccc4c3oc3ccccc34)n2)cc1 ; c1ccc(-c2nc(-c3ccc4c(c3)c3ccccc3n4-c3cccc4oc5cccc(-c6ccccc6)c5c34)nc(-c3cccc4oc5ccccc5c34)n2)cc1]

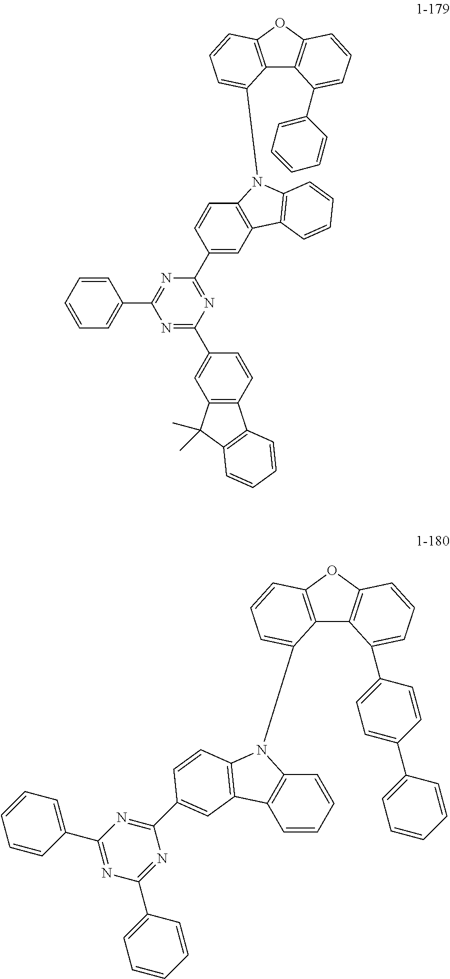 [SMILES: CC1(C)c2ccccc2-c2ccc(-c3nc(-c4ccccc4)nc(-c4ccc5c(c4)c4ccccc4n5-c4cccc5oc6cccc(-c7ccccc7)c6c45)n3)cc21 ; c1ccc(-c2ccc(-c3cccc4oc5cccc(-n6c7ccccc7c7cc(-c8nc(-c9ccccc9)nc(-c9ccccc9)n8)ccc76)c5c34)cc2)cc1]

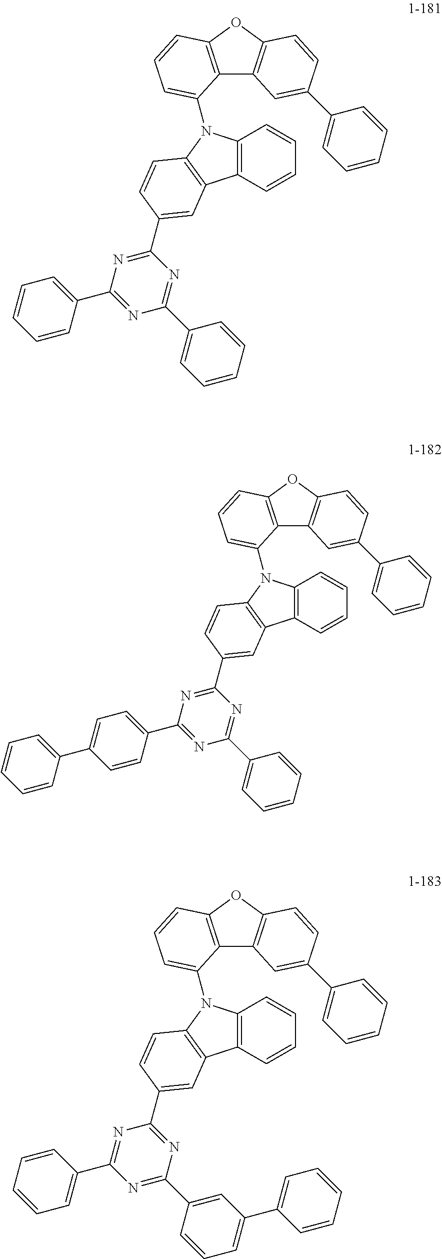 [SMILES: c1ccc(-c2ccc3oc4cccc(-n5c6ccccc6c6cc(-c7nc(-c8ccccc8)nc(-c8ccccc8)n7)ccc65)c4c3c2)cc1 ; c1ccc(-c2ccc(-c3nc(-c4ccccc4)nc(-c4ccc5c(c4)c4ccccc4n5-c4cccc5oc6ccc(-c7ccccc7)cc6c45)n3)cc2)cc1 ; c1ccc(-c2cccc(-c3nc(-c4ccccc4)nc(-c4ccc5c(c4)c4ccccc4n5-c4cccc5oc6ccc(-c7ccccc7)cc6c45)n3)c2)cc1]

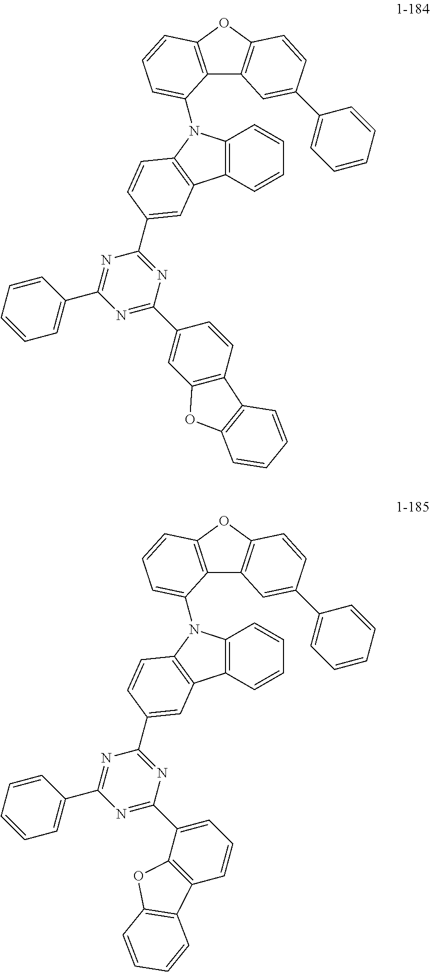 [SMILES: c1ccc(-c2ccc3oc4cccc(-n5c6ccccc6c6cc(-c7nc(-c8ccccc8)nc(-c8ccc9c(c8)oc8ccccc89)n7)ccc65)c4c3c2)cc1 ; c1ccc(-c2ccc3oc4cccc(-n5c6ccccc6c6cc(-c7nc(-c8ccccc8)nc(-c8cccc9c8oc8ccccc89)n7)ccc65)c4c3c2)cc1]

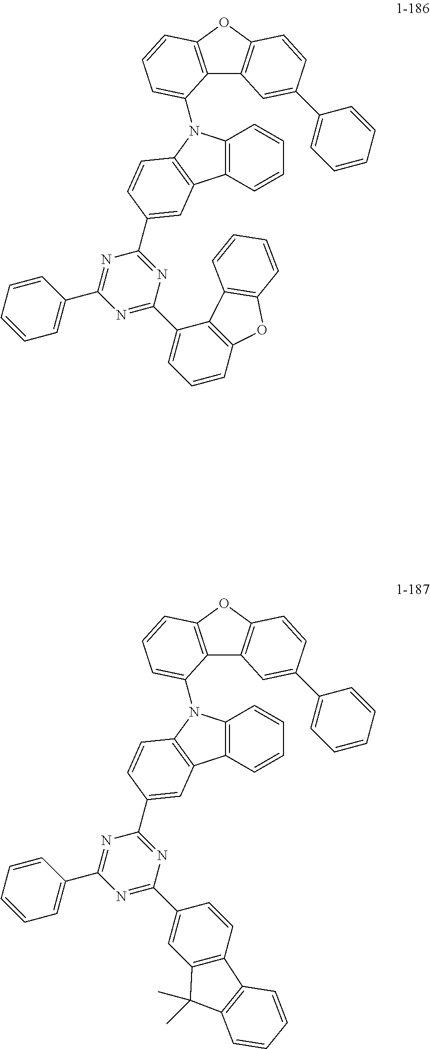 [SMILES: c1ccc(-c2ccc3oc4cccc(-n5c6ccccc6c6cc(-c7nc(-c8ccccc8)nc(-c8cccc9oc%10ccccc%10c89)n7)ccc65)c4c3c2)cc1 ; CC1(C)c2ccccc2-c2ccc(-c3nc(-c4ccccc4)nc(-c4ccc5c(c4)c4ccccc4n5-c4cccc5oc6ccc(-c7ccccc7)cc6c45)n3)cc21]

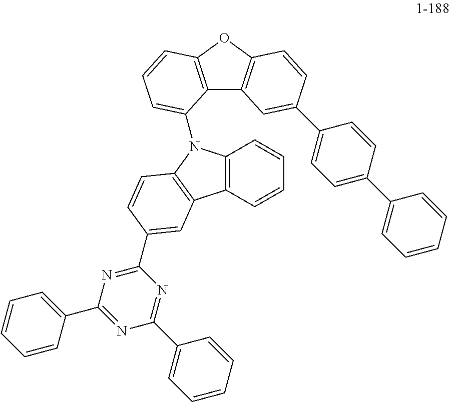 [SMILES: c1ccc(-c2ccc(-c3ccc4oc5cccc(-n6c7ccccc7c7cc(-c8nc(-c9ccccc9)nc(-c9ccccc9)n8)ccc76)c5c4c3)cc2)cc1]

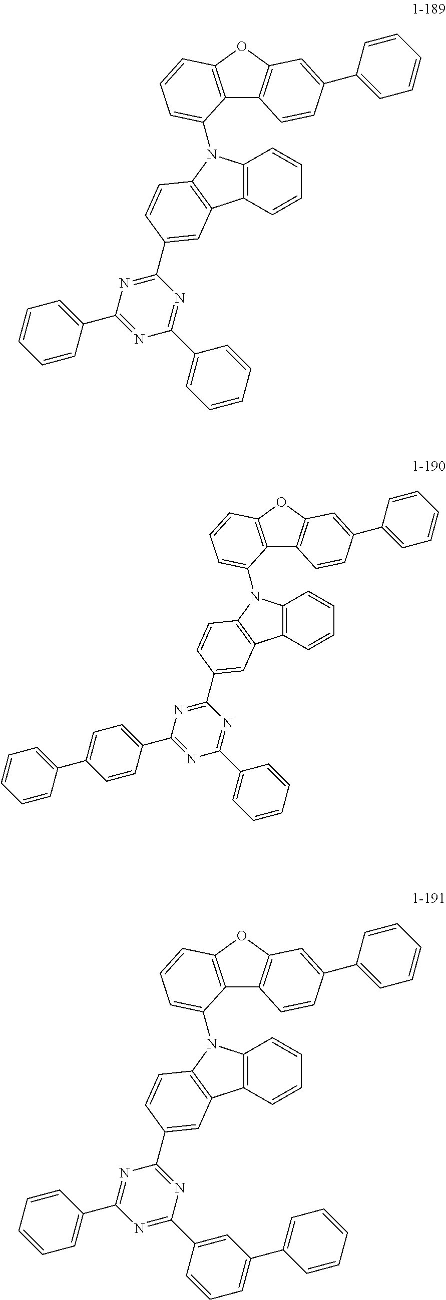 [SMILES: c1ccc(-c2ccc3c(c2)oc2cccc(-n4c5ccccc5c5cc(-c6nc(-c7ccccc7)nc(-c7ccccc7)n6)ccc54)c23)cc1 ; c1ccc(-c2ccc(-c3nc(-c4ccccc4)nc(-c4ccc5c(c4)c4ccccc4n5-c4cccc5oc6cc(-c7ccccc7)ccc6c45)n3)cc2)cc1 ; c1ccc(-c2cccc(-c3nc(-c4ccccc4)nc(-c4ccc5c(c4)c4ccccc4n5-c4cccc5oc6cc(-c7ccccc7)ccc6c45)n3)c2)cc1]

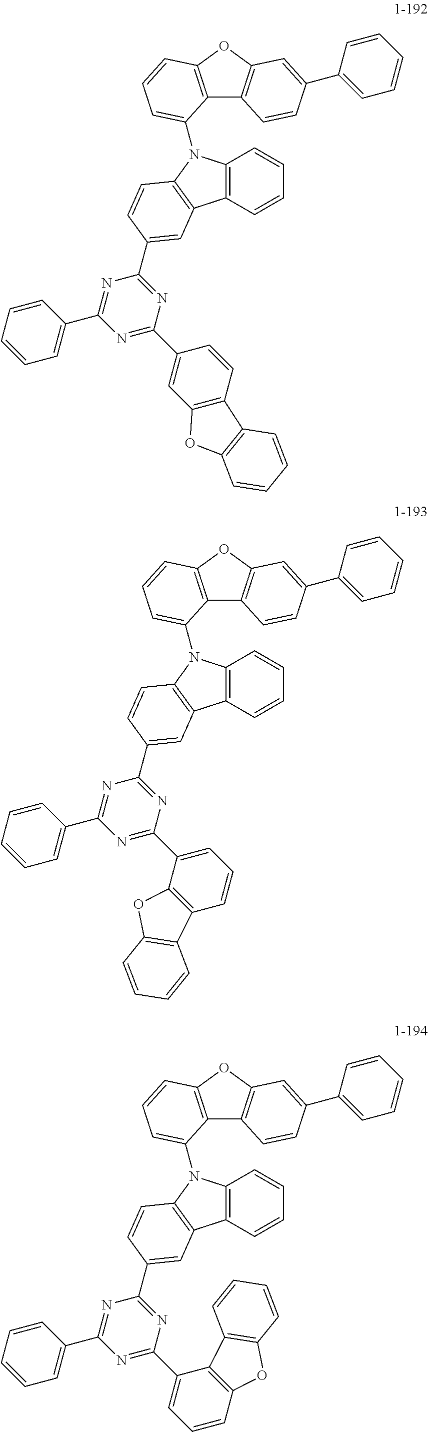 [SMILES: c1ccc(-c2ccc3c(c2)oc2cccc(-n4c5ccccc5c5cc(-c6nc(-c7ccccc7)nc(-c7ccc8c(c7)oc7ccccc78)n6)ccc54)c23)cc1 ; c1ccc(-c2ccc3c(c2)oc2cccc(-n4c5ccccc5c5cc(-c6nc(-c7ccccc7)nc(-c7cccc8c7oc7ccccc78)n6)ccc54)c23)cc1 ; c1ccc(-c2ccc3c(c2)oc2cccc(-n4c5ccccc5c5cc(-c6nc(-c7ccccc7)nc(-c7cccc8oc9ccccc9c78)n6)ccc54)c23)cc1]

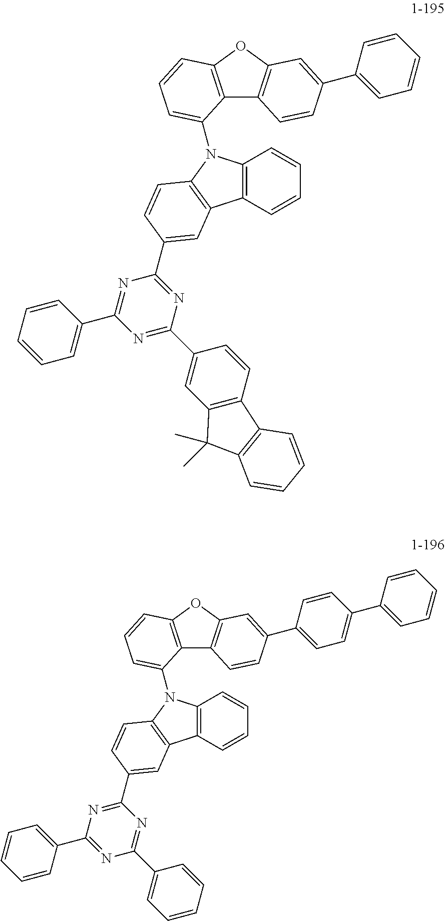 [SMILES: CC1(C)c2ccccc2-c2ccc(-c3nc(-c4ccccc4)nc(-c4ccc5c(c4)c4ccccc4n5-c4cccc5oc6cc(-c7ccccc7)ccc6c45)n3)cc21 ; c1ccc(-c2ccc(-c3ccc4c(c3)oc3cccc(-n5c6ccccc6c6cc(-c7nc(-c8ccccc8)nc(-c8ccccc8)n7)ccc65)c34)cc2)cc1]

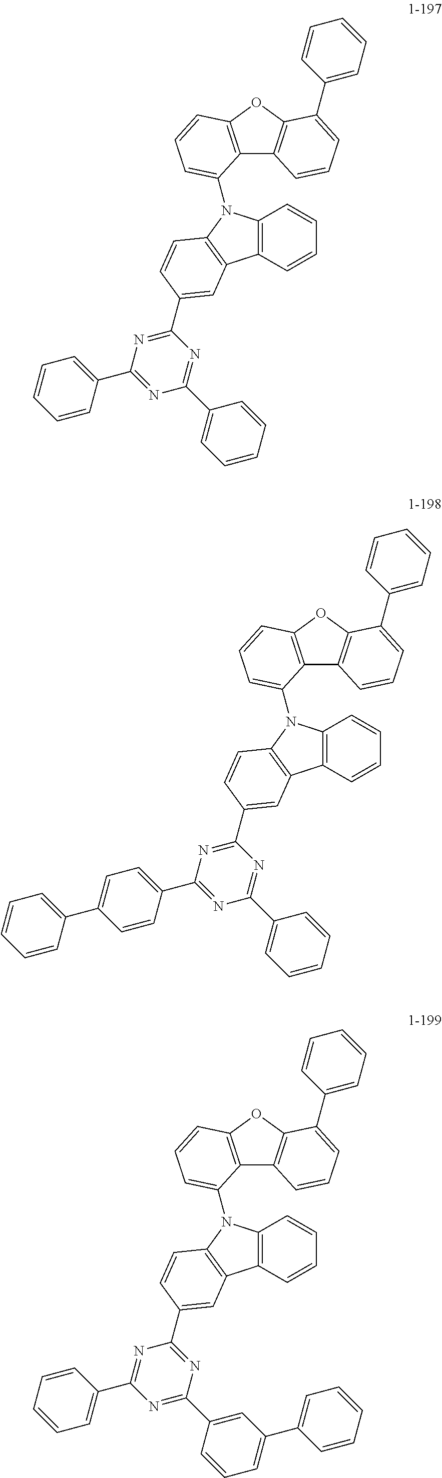 [SMILES: c1ccc(-c2nc(-c3ccccc3)nc(-c3ccc4c(c3)c3ccccc3n4-c3cccc4oc5c(-c6ccccc6)cccc5c34)n2)cc1 ; c1ccc(-c2ccc(-c3nc(-c4ccccc4)nc(-c4ccc5c(c4)c4ccccc4n5-c4cccc5oc6c(-c7ccccc7)cccc6c45)n3)cc2)cc1 ; c1ccc(-c2cccc(-c3nc(-c4ccccc4)nc(-c4ccc5c(c4)c4ccccc4n5-c4cccc5oc6c(-c7ccccc7)cccc6c45)n3)c2)cc1]

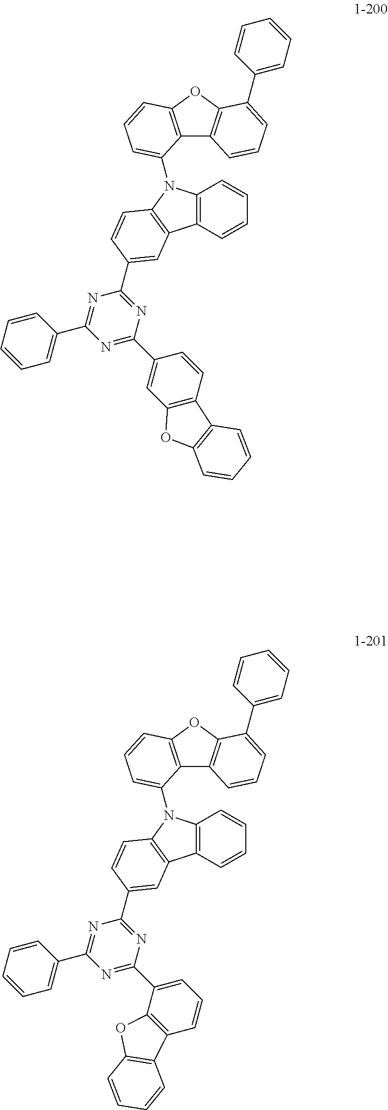 [SMILES: c1ccc(-c2nc(-c3ccc4c(c3)oc3ccccc34)nc(-c3ccc4c(c3)c3ccccc3n4-c3cccc4oc5c(-c6ccccc6)cccc5c34)n2)cc1 ; c1ccc(-c2nc(-c3ccc4c(c3)c3ccccc3n4-c3cccc4oc5c(-c6ccccc6)cccc5c34)nc(-c3cccc4c3oc3ccccc34)n2)cc1]

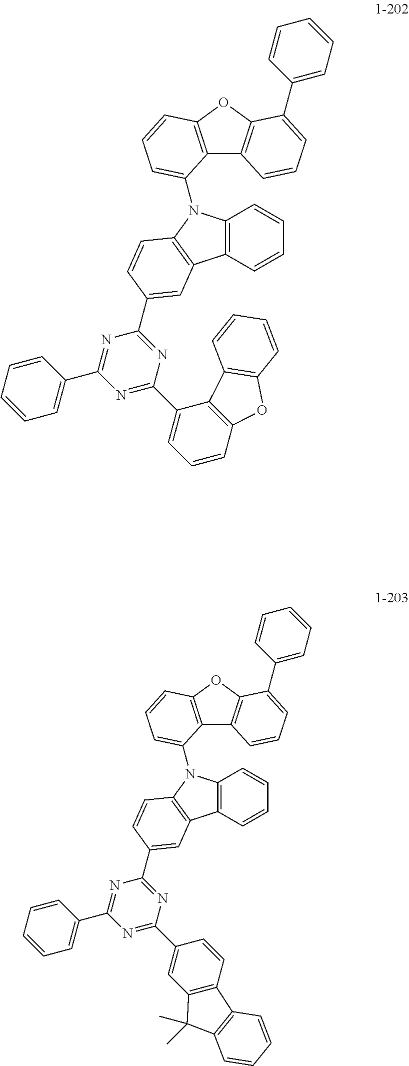 [SMILES: c1ccc(-c2nc(-c3ccc4c(c3)c3ccccc3n4-c3cccc4oc5c(-c6ccccc6)cccc5c34)nc(-c3cccc4oc5ccccc5c34)n2)cc1 ; CC1(C)c2ccccc2-c2ccc(-c3nc(-c4ccccc4)nc(-c4ccc5c(c4)c4ccccc4n5-c4cccc5oc6c(-c7ccccc7)cccc6c45)n3)cc21]

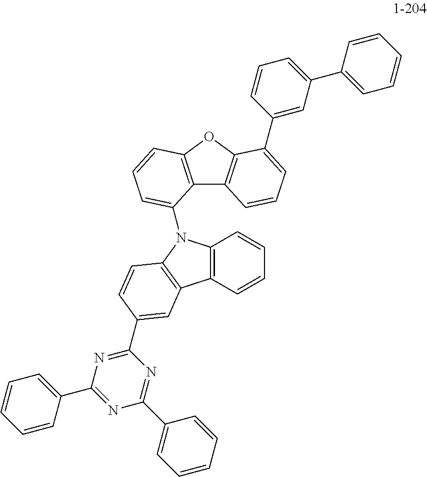 [SMILES: c1ccc(-c2cccc(-c3cccc4c3oc3cccc(-n5c6ccccc6c6cc(-c7nc(-c8ccccc8)nc(-c8ccccc8)n7)ccc65)c34)c2)cc1]

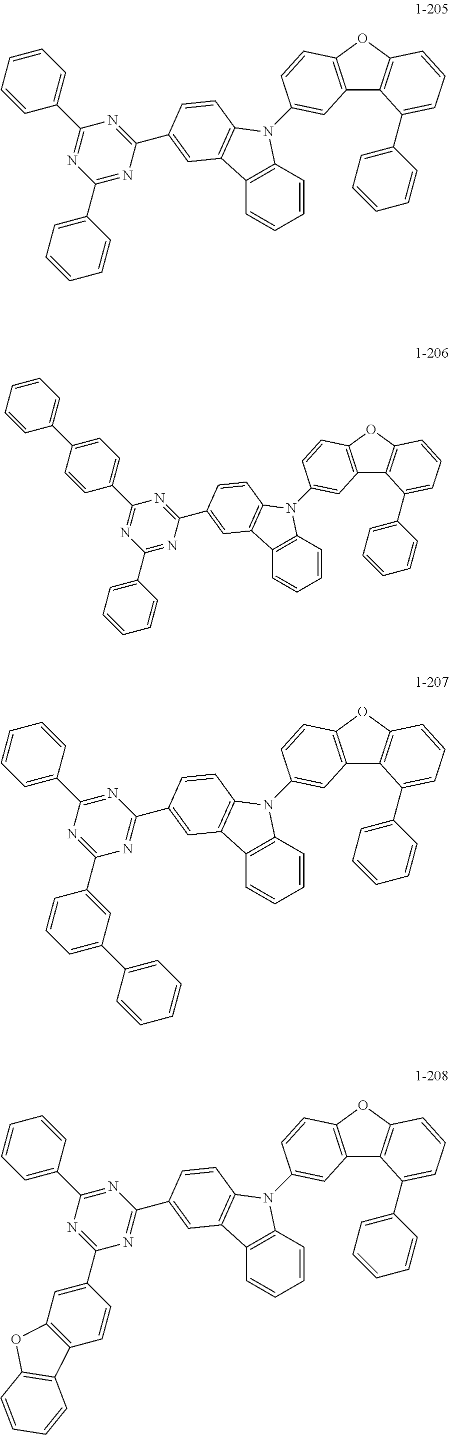 [SMILES: c1ccc(-c2nc(-c3ccccc3)nc(-c3ccc4c(c3)c3ccccc3n4-c3ccc4oc5cccc(-c6ccccc6)c5c4c3)n2)cc1 ; c1ccc(-c2ccc(-c3nc(-c4ccccc4)nc(-c4ccc5c(c4)c4ccccc4n5-c4ccc5oc6cccc(-c7ccccc7)c6c5c4)n3)cc2)cc1 ; c1ccc(-c2cccc(-c3nc(-c4ccccc4)nc(-c4ccc5c(c4)c4ccccc4n5-c4ccc5oc6cccc(-c7ccccc7)c6c5c4)n3)c2)cc1 ; c1ccc(-c2nc(-c3ccc4c(c3)oc3ccccc34)nc(-c3ccc4c(c3)c3ccccc3n4-c3ccc4oc5cccc(-c6ccccc6)c5c4c3)n2)cc1]

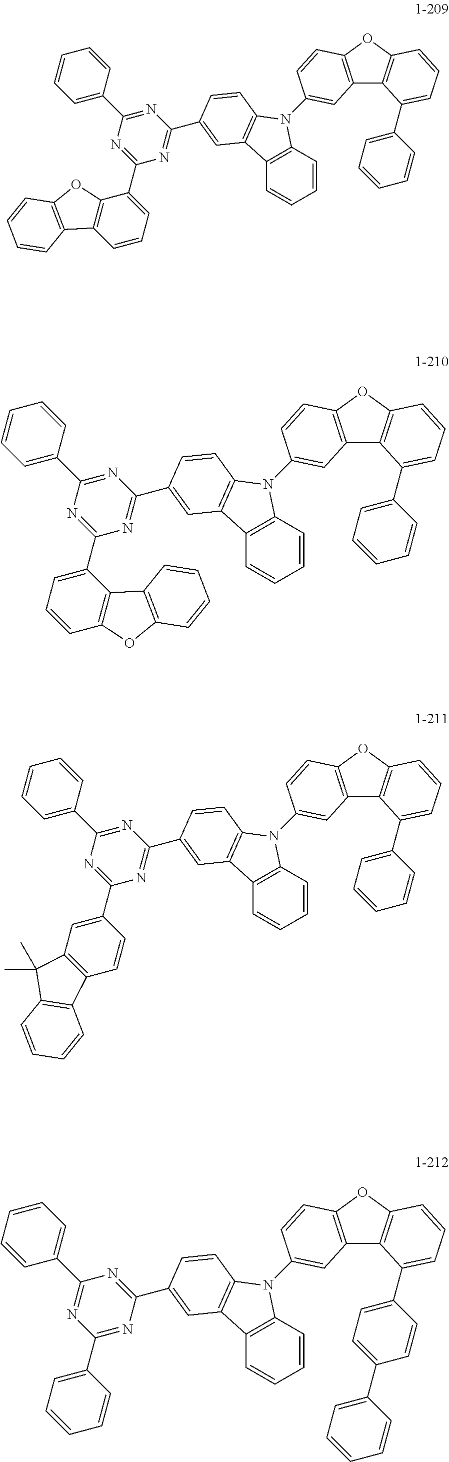 [SMILES: c1ccc(-c2nc(-c3ccc4c(c3)c3ccccc3n4-c3ccc4oc5cccc(-c6ccccc6)c5c4c3)nc(-c3cccc4c3oc3ccccc34)n2)cc1 ; c1ccc(-c2nc(-c3ccc4c(c3)c3ccccc3n4-c3ccc4oc5cccc(-c6ccccc6)c5c4c3)nc(-c3cccc4oc5ccccc5c34)n2)cc1 ; CC1(C)c2ccccc2-c2ccc(-c3nc(-c4ccccc4)nc(-c4ccc5c(c4)c4ccccc4n5-c4ccc5oc6cccc(-c7ccccc7)c6c5c4)n3)cc21 ; c1ccc(-c2ccc(-c3cccc4oc5ccc(-n6c7ccccc7c7cc(-c8nc(-c9ccccc9)nc(-c9ccccc9)n8)ccc76)cc5c34)cc2)cc1]

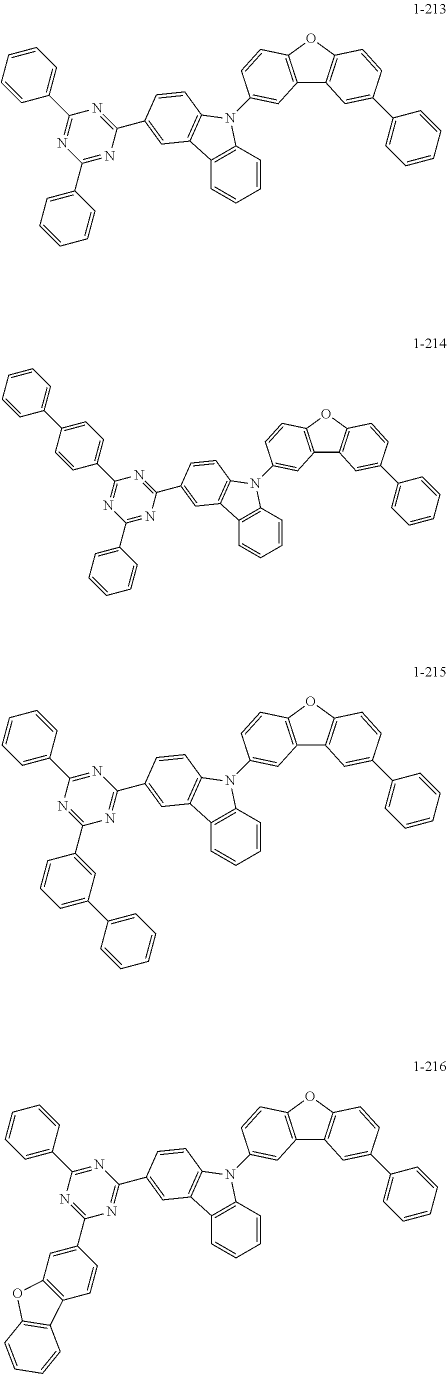 [SMILES: c1ccc(-c2ccc3oc4ccc(-n5c6ccccc6c6cc(-c7nc(-c8ccccc8)nc(-c8ccccc8)n7)ccc65)cc4c3c2)cc1 ; c1ccc(-c2ccc(-c3nc(-c4ccccc4)nc(-c4ccc5c(c4)c4ccccc4n5-c4ccc5oc6ccc(-c7ccccc7)cc6c5c4)n3)cc2)cc1 ; c1ccc(-c2cccc(-c3nc(-c4ccccc4)nc(-c4ccc5c(c4)c4ccccc4n5-c4ccc5oc6ccc(-c7ccccc7)cc6c5c4)n3)c2)cc1 ; c1ccc(-c2ccc3oc4ccc(-n5c6ccccc6c6cc(-c7nc(-c8ccccc8)nc(-c8ccc9c(c8)oc8ccccc89)n7)ccc65)cc4c3c2)cc1]

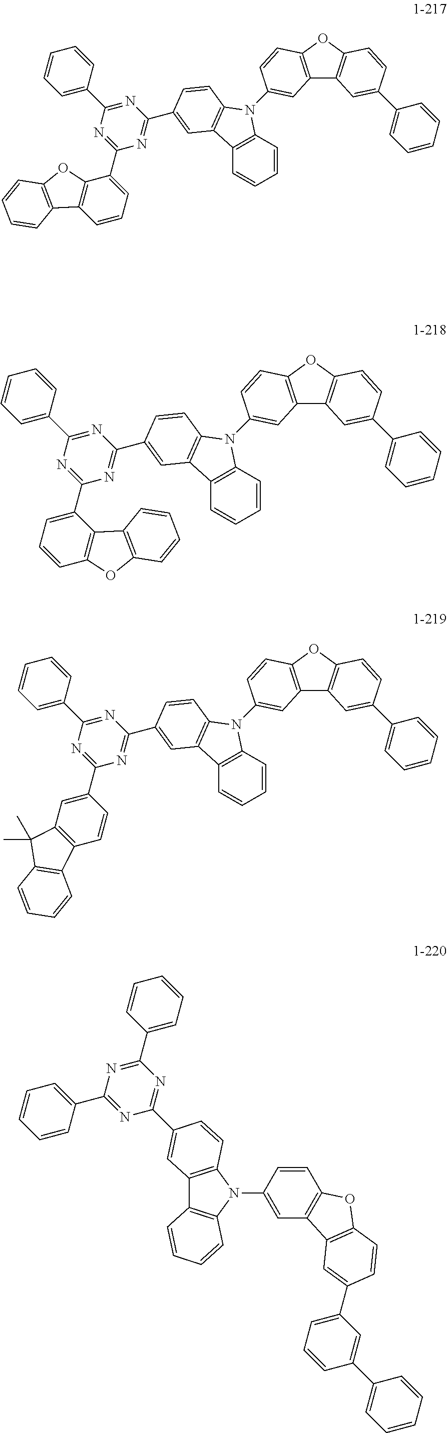 [SMILES: c1ccc(-c2ccc3oc4ccc(-n5c6ccccc6c6cc(-c7nc(-c8ccccc8)nc(-c8cccc9c8oc8ccccc89)n7)ccc65)cc4c3c2)cc1 ; c1ccc(-c2ccc3oc4ccc(-n5c6ccccc6c6cc(-c7nc(-c8ccccc8)nc(-c8cccc9oc%10ccccc%10c89)n7)ccc65)cc4c3c2)cc1 ; CC1(C)c2ccccc2-c2ccc(-c3nc(-c4ccccc4)nc(-c4ccc5c(c4)c4ccccc4n5-c4ccc5oc6ccc(-c7ccccc7)cc6c5c4)n3)cc21 ; c1ccc(-c2cccc(-c3ccc4oc5ccc(-n6c7ccccc7c7cc(-c8nc(-c9ccccc9)nc(-c9ccccc9)n8)ccc76)cc5c4c3)c2)cc1]

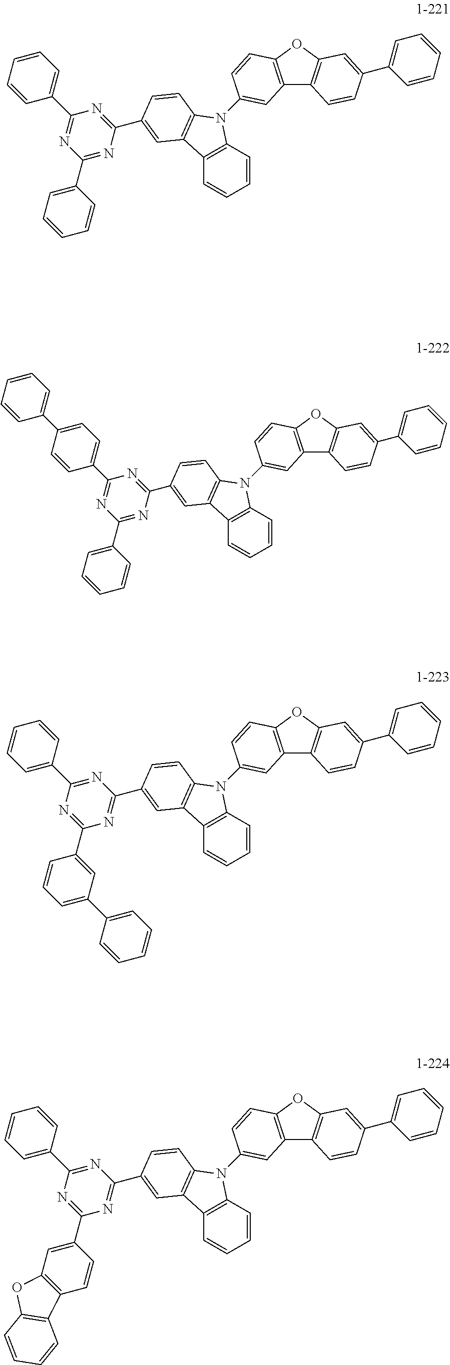 [SMILES: c1ccc(-c2ccc3c(c2)oc2ccc(-n4c5ccccc5c5cc(-c6nc(-c7ccccc7)nc(-c7ccccc7)n6)ccc54)cc23)cc1 ; c1ccc(-c2ccc(-c3nc(-c4ccccc4)nc(-c4ccc5c(c4)c4ccccc4n5-c4ccc5oc6cc(-c7ccccc7)ccc6c5c4)n3)cc2)cc1 ; c1ccc(-c2cccc(-c3nc(-c4ccccc4)nc(-c4ccc5c(c4)c4ccccc4n5-c4ccc5oc6cc(-c7ccccc7)ccc6c5c4)n3)c2)cc1 ; c1ccc(-c2ccc3c(c2)oc2ccc(-n4c5ccccc5c5cc(-c6nc(-c7ccccc7)nc(-c7ccc8c(c7)oc7ccccc78)n6)ccc54)cc23)cc1]

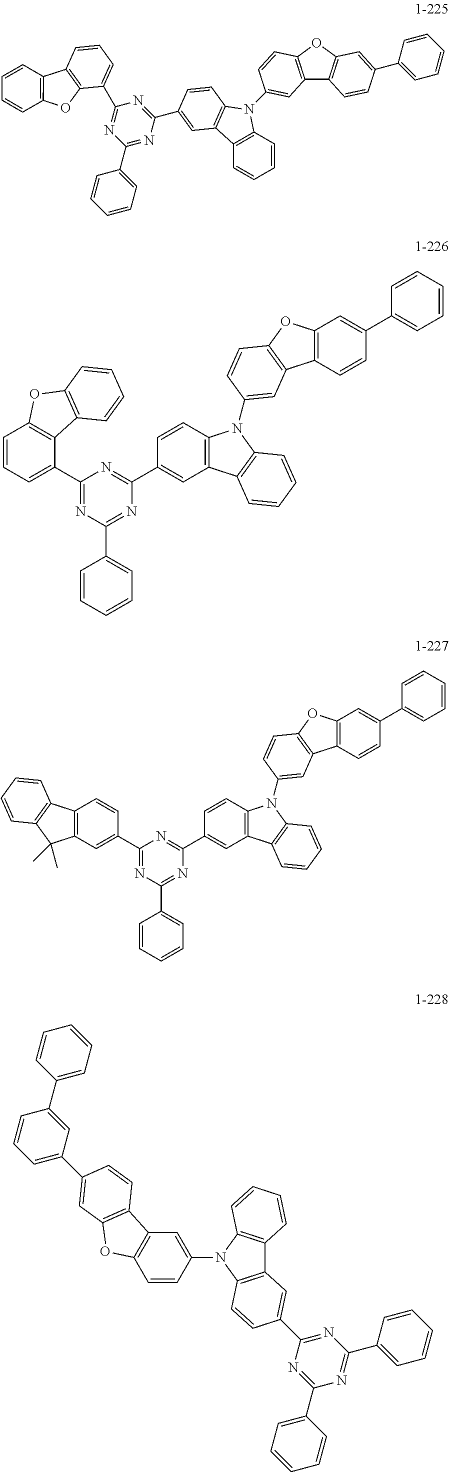 [SMILES: c1ccc(-c2ccc3c(c2)oc2ccc(-n4c5ccccc5c5cc(-c6nc(-c7ccccc7)nc(-c7cccc8c7oc7ccccc78)n6)ccc54)cc23)cc1 ; c1ccc(-c2ccc3c(c2)oc2ccc(-n4c5ccccc5c5cc(-c6nc(-c7ccccc7)nc(-c7cccc8oc9ccccc9c78)n6)ccc54)cc23)cc1 ; CC1(C)c2ccccc2-c2ccc(-c3nc(-c4ccccc4)nc(-c4ccc5c(c4)c4ccccc4n5-c4ccc5oc6cc(-c7ccccc7)ccc6c5c4)n3)cc21 ; c1ccc(-c2cccc(-c3ccc4c(c3)oc3ccc(-n5c6ccccc6c6cc(-c7nc(-c8ccccc8)nc(-c8ccccc8)n7)ccc65)cc34)c2)cc1]

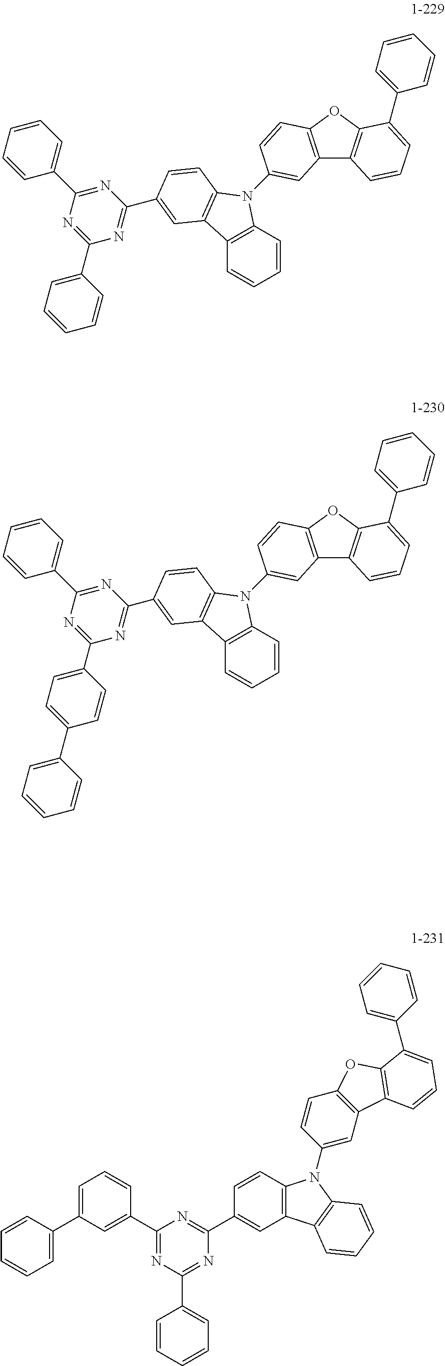 [SMILES: c1ccc(-c2nc(-c3ccccc3)nc(-c3ccc4c(c3)c3ccccc3n4-c3ccc4oc5c(-c6ccccc6)cccc5c4c3)n2)cc1 ; c1ccc(-c2ccc(-c3nc(-c4ccccc4)nc(-c4ccc5c(c4)c4ccccc4n5-c4ccc5oc6c(-c7ccccc7)cccc6c5c4)n3)cc2)cc1 ; c1ccc(-c2cccc(-c3nc(-c4ccccc4)nc(-c4ccc5c(c4)c4ccccc4n5-c4ccc5oc6c(-c7ccccc7)cccc6c5c4)n3)c2)cc1]

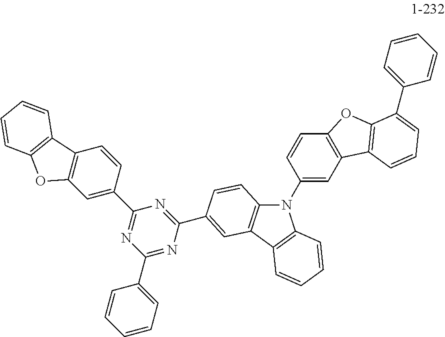 [SMILES: c1ccc(-c2nc(-c3ccc4c(c3)oc3ccccc34)nc(-c3ccc4c(c3)c3ccccc3n4-c3ccc4oc5c(-c6ccccc6)cccc5c4c3)n2)cc1]

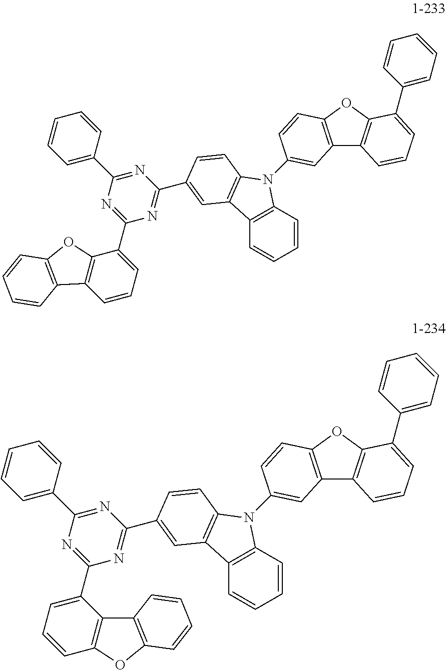 [SMILES: c1ccc(-c2nc(-c3ccc4c(c3)c3ccccc3n4-c3ccc4oc5c(-c6ccccc6)cccc5c4c3)nc(-c3cccc4c3oc3ccccc34)n2)cc1 ; c1ccc(-c2nc(-c3ccc4c(c3)c3ccccc3n4-c3ccc4oc5c(-c6ccccc6)cccc5c4c3)nc(-c3cccc4oc5ccccc5c34)n2)cc1]

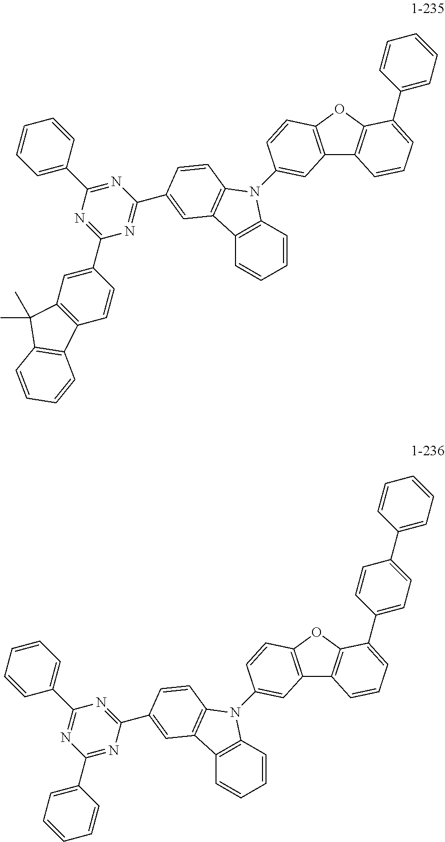 [SMILES: CC1(C)c2ccccc2-c2ccc(-c3nc(-c4ccccc4)nc(-c4ccc5c(c4)c4ccccc4n5-c4ccc5oc6c(-c7ccccc7)cccc6c5c4)n3)cc21 ; c1ccc(-c2ccc(-c3cccc4c3oc3ccc(-n5c6ccccc6c6cc(-c7nc(-c8ccccc8)nc(-c8ccccc8)n7)ccc65)cc34)cc2)cc1]

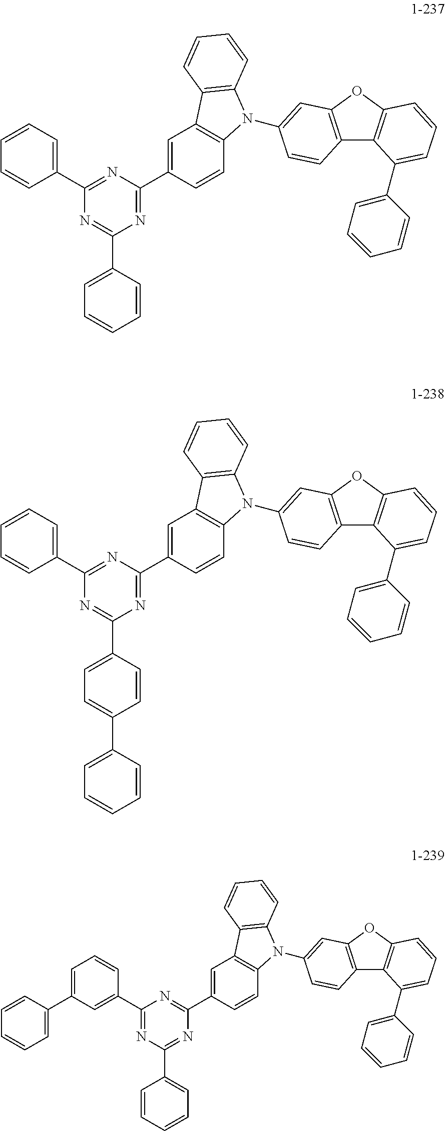 [SMILES: c1ccc(-c2nc(-c3ccccc3)nc(-c3ccc4c(c3)c3ccccc3n4-c3ccc4c(c3)oc3cccc(-c5ccccc5)c34)n2)cc1 ; c1ccc(-c2ccc(-c3nc(-c4ccccc4)nc(-c4ccc5c(c4)c4ccccc4n5-c4ccc5c(c4)oc4cccc(-c6ccccc6)c45)n3)cc2)cc1 ; c1ccc(-c2cccc(-c3nc(-c4ccccc4)nc(-c4ccc5c(c4)c4ccccc4n5-c4ccc5c(c4)oc4cccc(-c6ccccc6)c45)n3)c2)cc1]

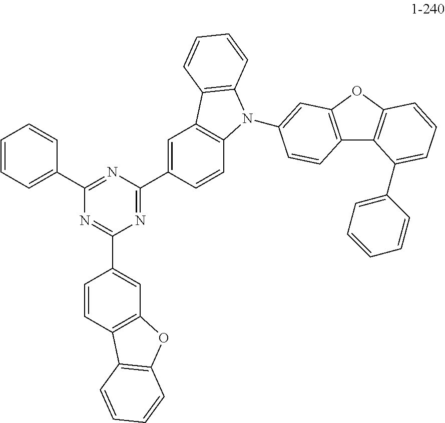 [SMILES: c1ccc(-c2nc(-c3ccc4c(c3)oc3ccccc34)nc(-c3ccc4c(c3)c3ccccc3n4-c3ccc4c(c3)oc3cccc(-c5ccccc5)c34)n2)cc1]

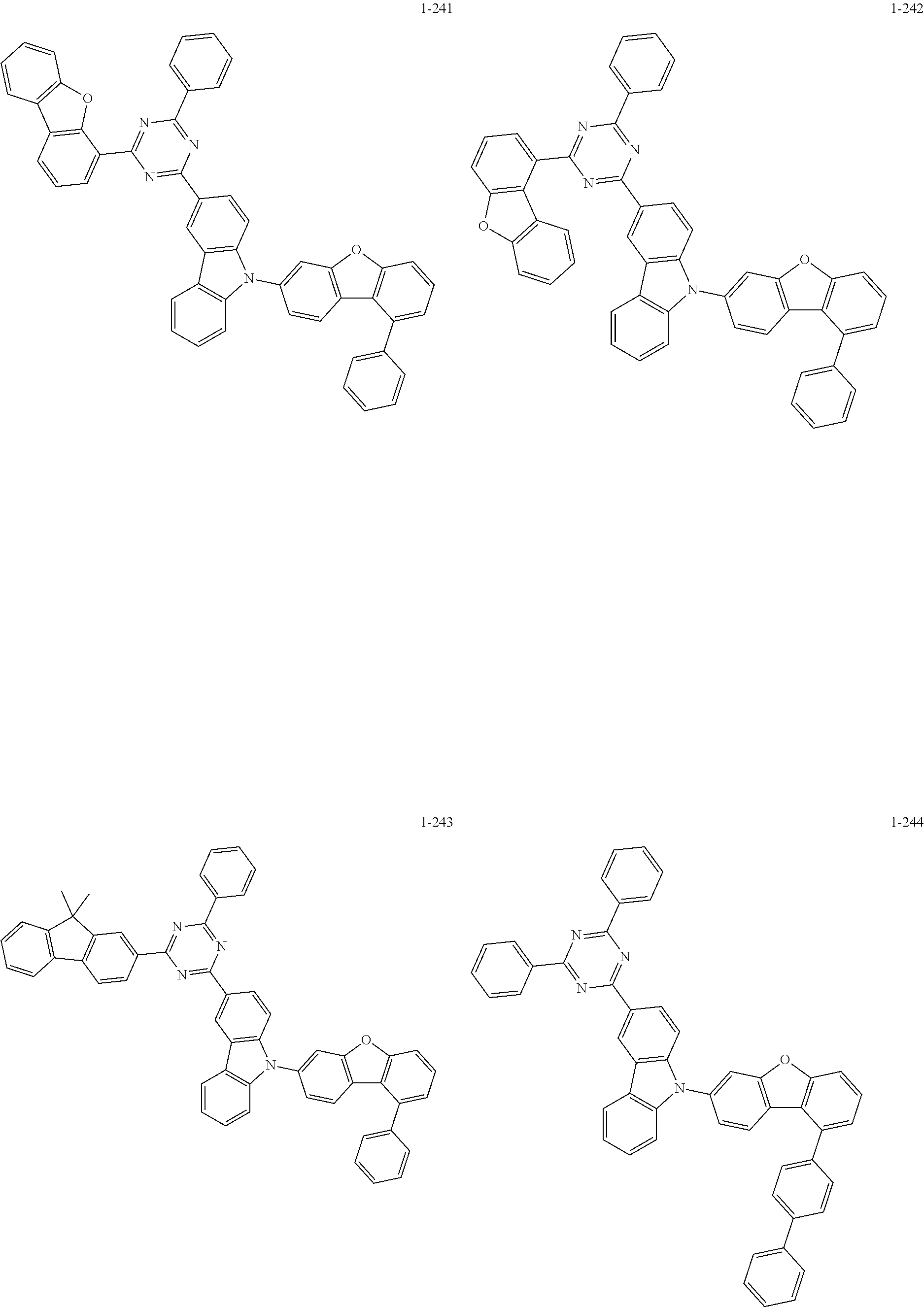 [SMILES: c1ccc(-c2nc(-c3ccc4c(c3)c3ccccc3n4-c3ccc4c(c3)oc3cccc(-c5ccccc5)c34)nc(-c3cccc4c3oc3ccccc34)n2)cc1 ; c1ccc(-c2nc(-c3ccc4c(c3)c3ccccc3n4-c3ccc4c(c3)oc3cccc(-c5ccccc5)c34)nc(-c3cccc4oc5ccccc5c34)n2)cc1 ; CC1(C)c2ccccc2-c2ccc(-c3nc(-c4ccccc4)nc(-c4ccc5c(c4)c4ccccc4n5-c4ccc5c(c4)oc4cccc(-c6ccccc6)c45)n3)cc21 ; c1ccc(-c2ccc(-c3cccc4oc5cc(-n6c7ccccc7c7cc(-c8nc(-c9ccccc9)nc(-c9ccccc9)n8)ccc76)ccc5c34)cc2)cc1]

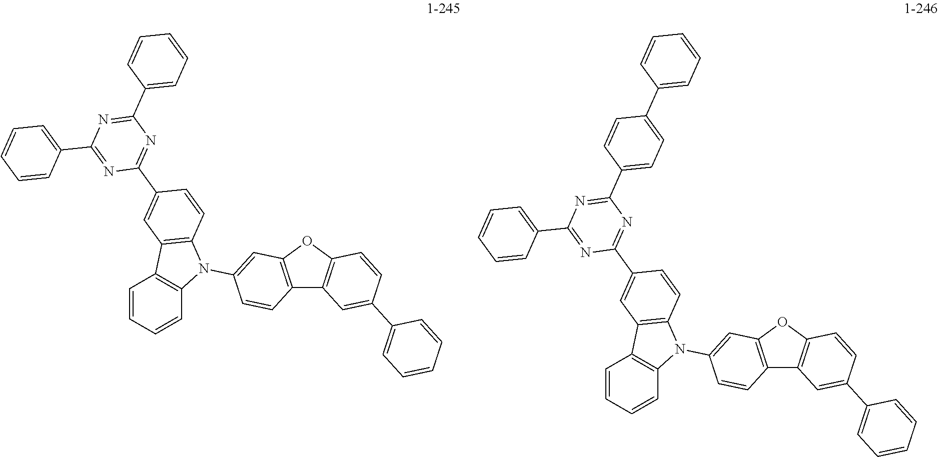 [SMILES: c1ccc(-c2ccc3oc4cc(-n5c6ccccc6c6cc(-c7nc(-c8ccccc8)nc(-c8ccccc8)n7)ccc65)ccc4c3c2)cc1 ; c1ccc(-c2ccc(-c3nc(-c4ccccc4)nc(-c4ccc5c(c4)c4ccccc4n5-c4ccc5c(c4)oc4ccc(-c6ccccc6)cc45)n3)cc2)cc1]

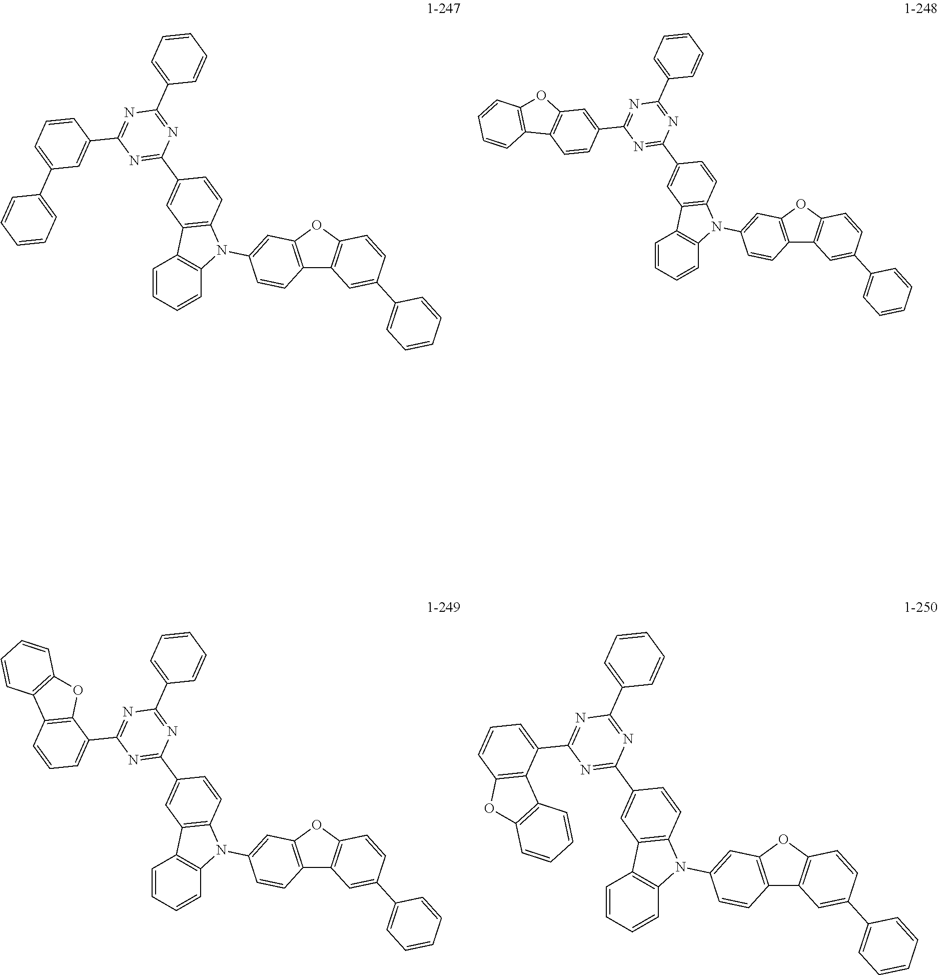 [SMILES: c1ccc(-c2cccc(-c3nc(-c4ccccc4)nc(-c4ccc5c(c4)c4ccccc4n5-c4ccc5c(c4)oc4ccc(-c6ccccc6)cc45)n3)c2)cc1 ; c1ccc(-c2ccc3oc4cc(-n5c6ccccc6c6cc(-c7nc(-c8ccccc8)nc(-c8ccc9c(c8)oc8ccccc89)n7)ccc65)ccc4c3c2)cc1 ; c1ccc(-c2ccc3oc4cc(-n5c6ccccc6c6cc(-c7nc(-c8ccccc8)nc(-c8cccc9c8oc8ccccc89)n7)ccc65)ccc4c3c2)cc1 ; c1ccc(-c2ccc3oc4cc(-n5c6ccccc6c6cc(-c7nc(-c8ccccc8)nc(-c8cccc9oc%10ccccc%10c89)n7)ccc65)ccc4c3c2)cc1]

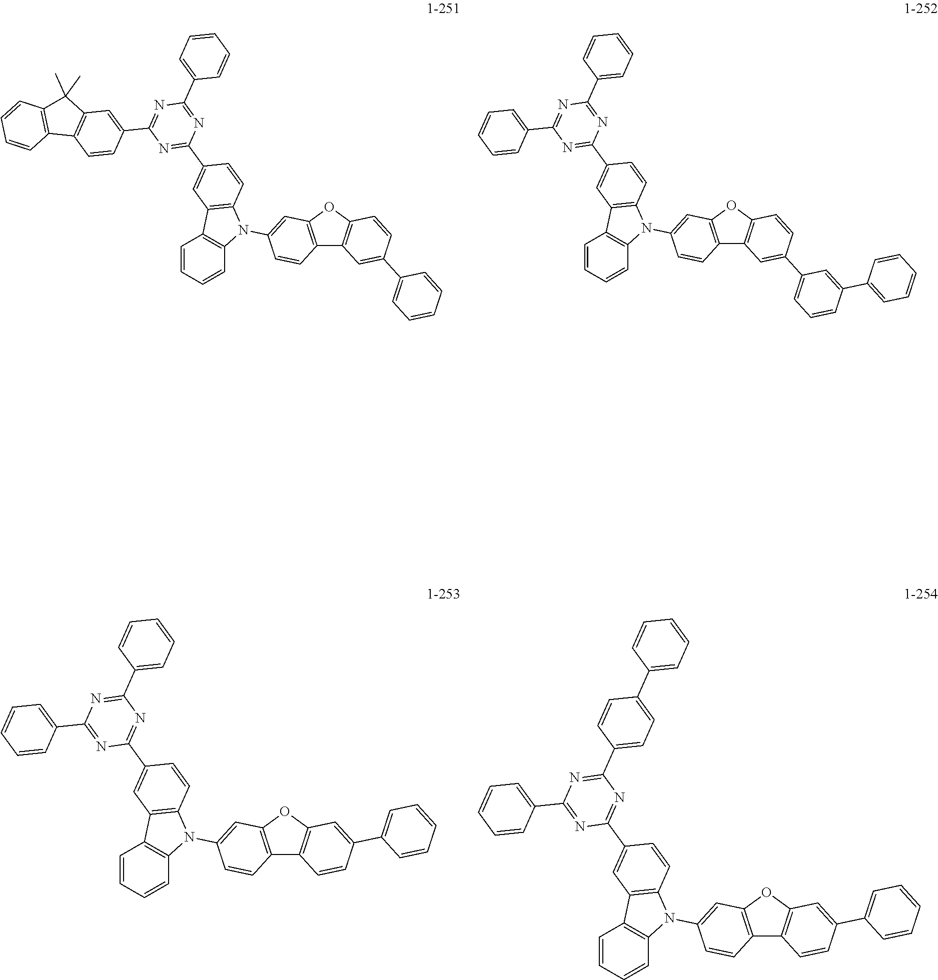 [SMILES: CC1(C)c2ccccc2-c2ccc(-c3nc(-c4ccccc4)nc(-c4ccc5c(c4)c4ccccc4n5-c4ccc5c(c4)oc4ccc(-c6ccccc6)cc45)n3)cc21 ; c1ccc(-c2cccc(-c3ccc4oc5cc(-n6c7ccccc7c7cc(-c8nc(-c9ccccc9)nc(-c9ccccc9)n8)ccc76)ccc5c4c3)c2)cc1 ; c1ccc(-c2ccc3c(c2)oc2cc(-n4c5ccccc5c5cc(-c6nc(-c7ccccc7)nc(-c7ccccc7)n6)ccc54)ccc23)cc1 ; c1ccc(-c2ccc(-c3nc(-c4ccccc4)nc(-c4ccc5c(c4)c4ccccc4n5-c4ccc5c(c4)oc4cc(-c6ccccc6)ccc45)n3)cc2)cc1]

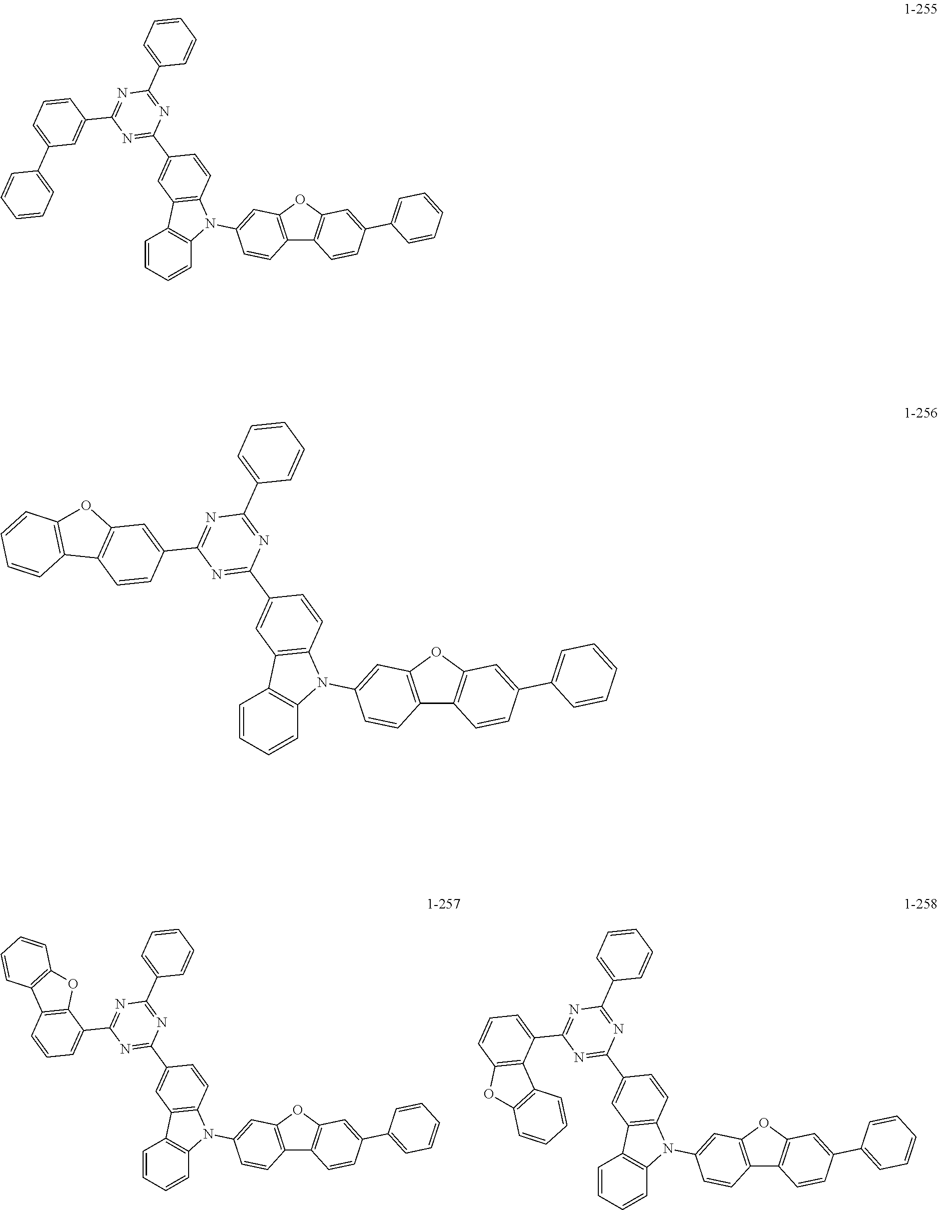 [SMILES: c1ccc(-c2cccc(-c3nc(-c4ccccc4)nc(-c4ccc5c(c4)c4ccccc4n5-c4ccc5c(c4)oc4cc(-c6ccccc6)ccc45)n3)c2)cc1 ; c1ccc(-c2ccc3c(c2)oc2cc(-n4c5ccccc5c5cc(-c6nc(-c7ccccc7)nc(-c7ccc8c(c7)oc7ccccc78)n6)ccc54)ccc23)cc1 ; c1ccc(-c2ccc3c(c2)oc2cc(-n4c5ccccc5c5cc(-c6nc(-c7ccccc7)nc(-c7cccc8c7oc7ccccc78)n6)ccc54)ccc23)cc1 ; c1ccc(-c2ccc3c(c2)oc2cc(-n4c5ccccc5c5cc(-c6nc(-c7ccccc7)nc(-c7cccc8oc9ccccc9c78)n6)ccc54)ccc23)cc1]

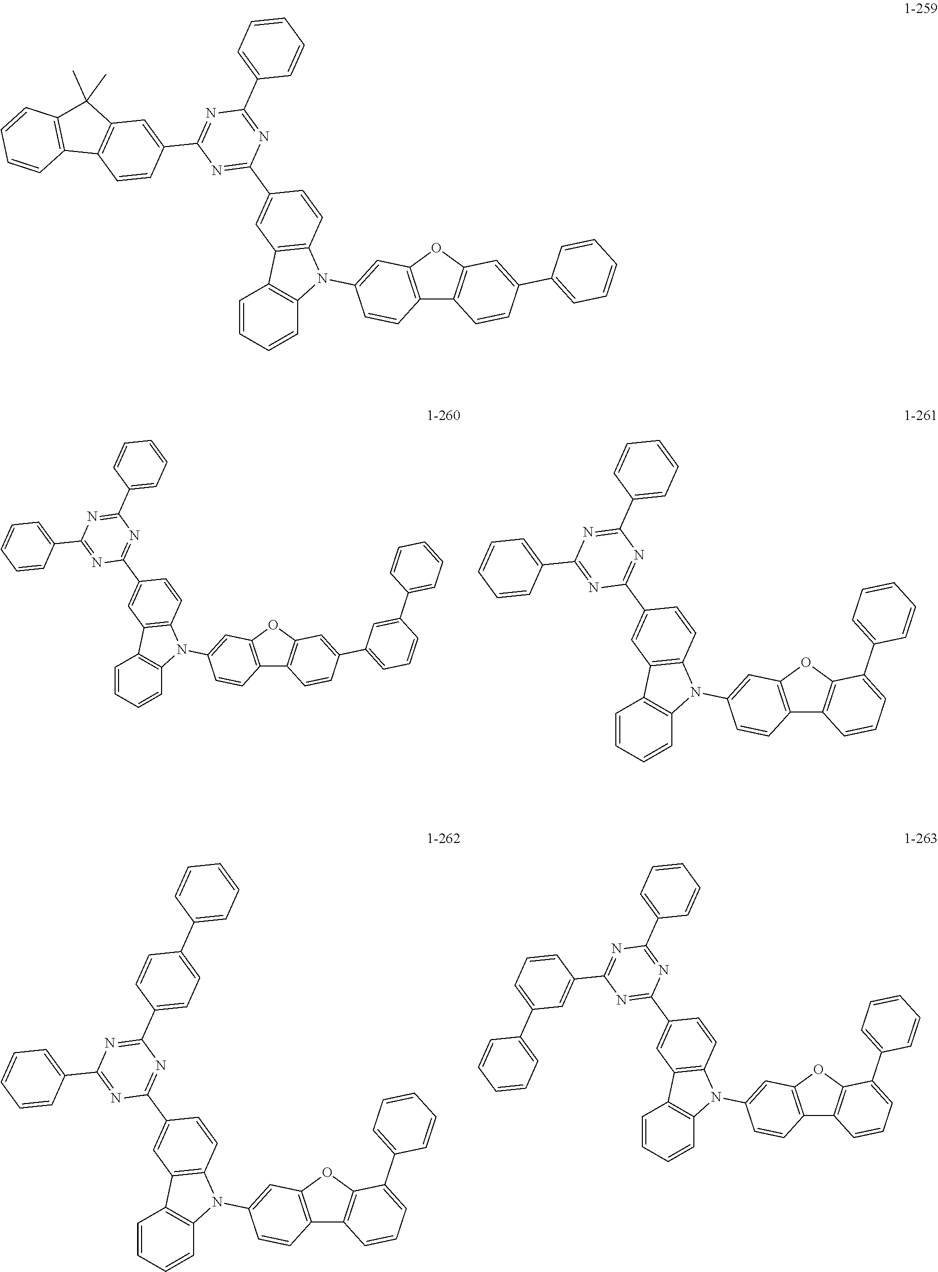

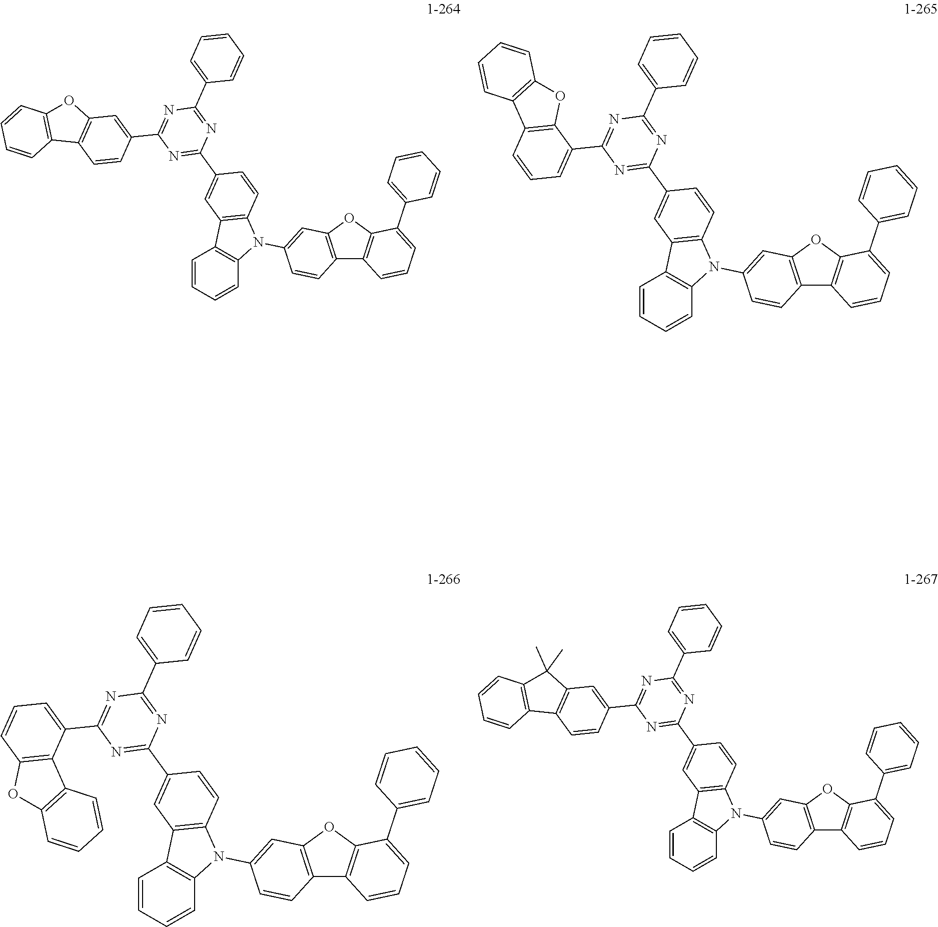 [SMILES: c1ccc(-c2nc(-c3ccc4c(c3)oc3ccccc34)nc(-c3ccc4c(c3)c3ccccc3n4-c3ccc4c(c3)oc3c(-c5ccccc5)cccc34)n2)cc1 ; c1ccc(-c2nc(-c3ccc4c(c3)c3ccccc3n4-c3ccc4c(c3)oc3c(-c5ccccc5)cccc34)nc(-c3cccc4c3oc3ccccc34)n2)cc1 ; c1ccc(-c2nc(-c3ccc4c(c3)c3ccccc3n4-c3ccc4c(c3)oc3c(-c5ccccc5)cccc34)nc(-c3cccc4oc5ccccc5c34)n2)cc1 ; CC1(C)c2ccccc2-c2ccc(-c3nc(-c4ccccc4)nc(-c4ccc5c(c4)c4ccccc4n5-c4ccc5c(c4)oc4c(-c6ccccc6)cccc45)n3)cc21]

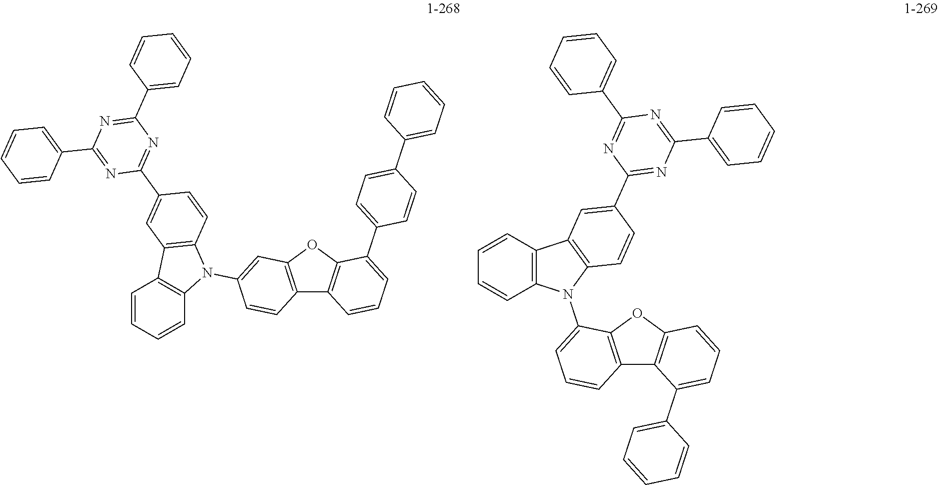 [SMILES: c1ccc(-c2ccc(-c3cccc4c3oc3cc(-n5c6ccccc6c6cc(-c7nc(-c8ccccc8)nc(-c8ccccc8)n7)ccc65)ccc34)cc2)cc1 ; c1ccc(-c2nc(-c3ccccc3)nc(-c3ccc4c(c3)c3ccccc3n4-c3cccc4c3oc3cccc(-c5ccccc5)c34)n2)cc1]

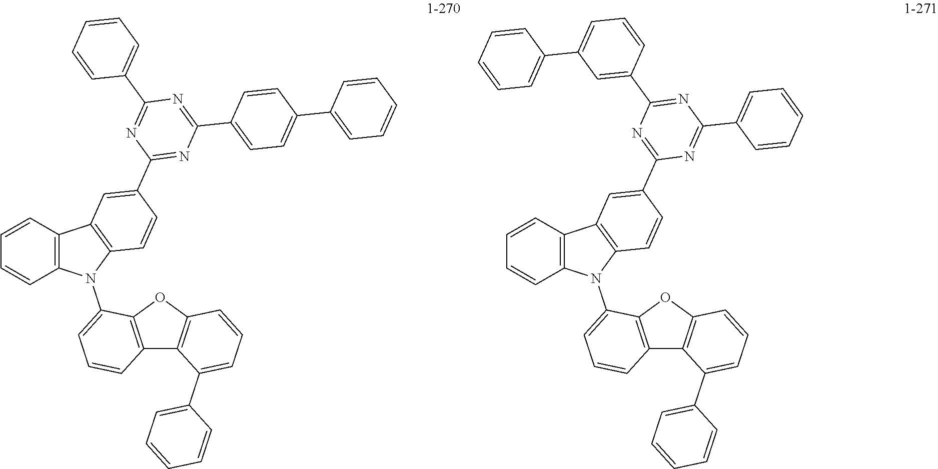 [SMILES: c1ccc(-c2ccc(-c3nc(-c4ccccc4)nc(-c4ccc5c(c4)c4ccccc4n5-c4cccc5c4oc4cccc(-c6ccccc6)c45)n3)cc2)cc1 ; c1ccc(-c2cccc(-c3nc(-c4ccccc4)nc(-c4ccc5c(c4)c4ccccc4n5-c4cccc5c4oc4cccc(-c6ccccc6)c45)n3)c2)cc1]

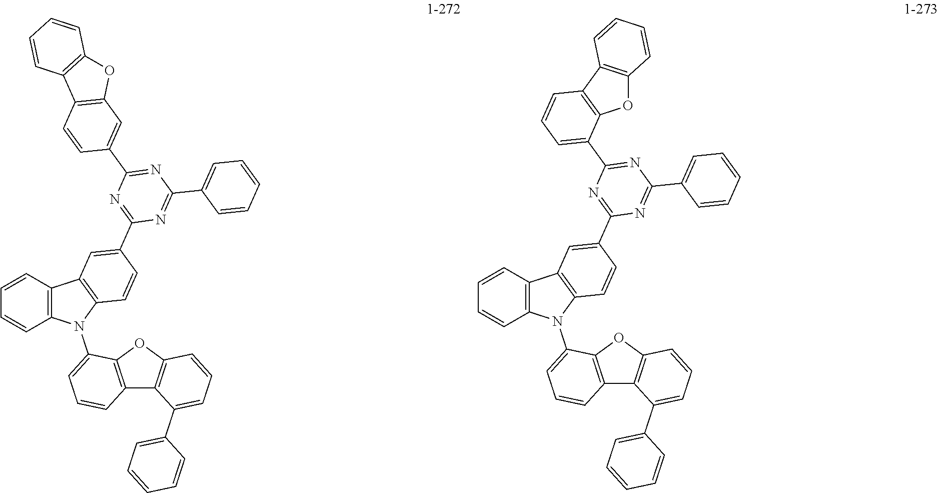 [SMILES: c1ccc(-c2nc(-c3ccc4c(c3)oc3ccccc34)nc(-c3ccc4c(c3)c3ccccc3n4-c3cccc4c3oc3cccc(-c5ccccc5)c34)n2)cc1 ; c1ccc(-c2nc(-c3ccc4c(c3)c3ccccc3n4-c3cccc4c3oc3cccc(-c5ccccc5)c34)nc(-c3cccc4c3oc3ccccc34)n2)cc1]

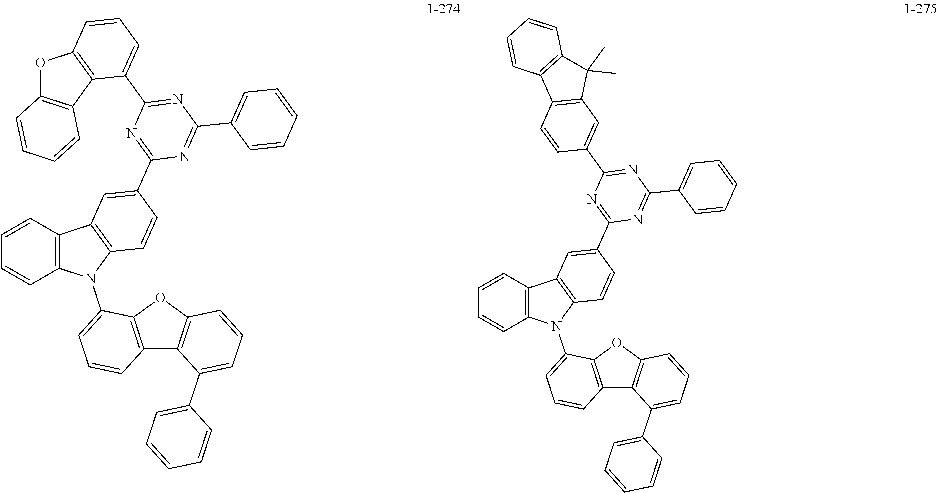 [SMILES: c1ccc(-c2nc(-c3ccc4c(c3)c3ccccc3n4-c3cccc4c3oc3cccc(-c5ccccc5)c34)nc(-c3cccc4oc5ccccc5c34)n2)cc1 ; CC1(C)c2ccccc2-c2ccc(-c3nc(-c4ccccc4)nc(-c4ccc5c(c4)c4ccccc4n5-c4cccc5c4oc4cccc(-c6ccccc6)c45)n3)cc21]

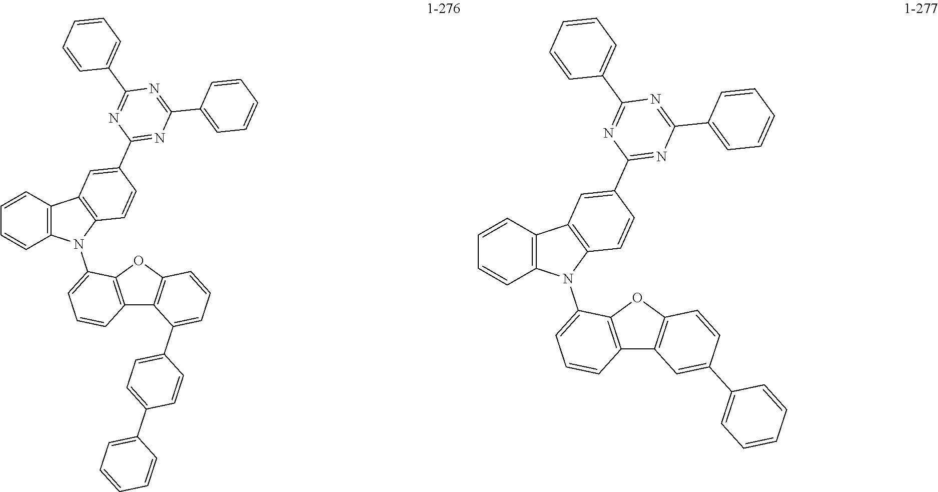 [SMILES: c1ccc(-c2ccc(-c3cccc4oc5c(-n6c7ccccc7c7cc(-c8nc(-c9ccccc9)nc(-c9ccccc9)n8)ccc76)cccc5c34)cc2)cc1 ; c1ccc(-c2ccc3oc4c(-n5c6ccccc6c6cc(-c7nc(-c8ccccc8)nc(-c8ccccc8)n7)ccc65)cccc4c3c2)cc1]

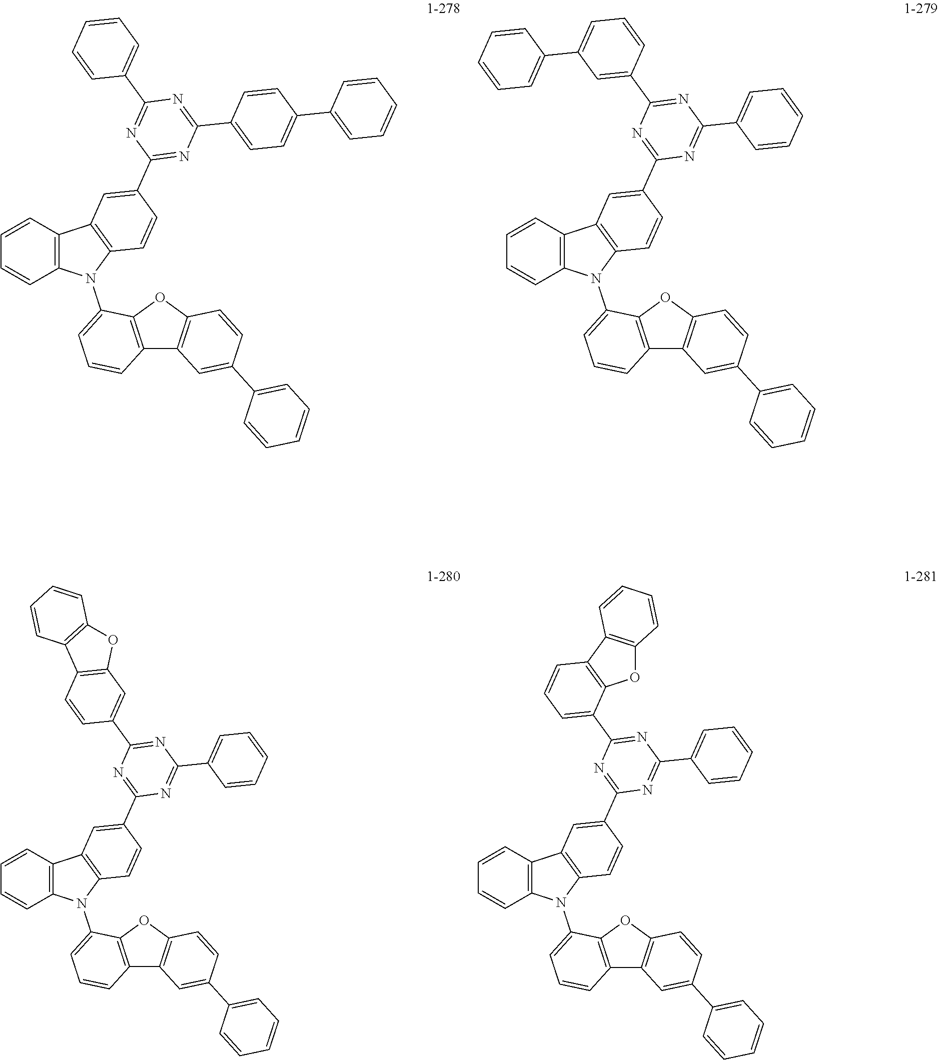 [SMILES: c1ccc(-c2ccc(-c3nc(-c4ccccc4)nc(-c4ccc5c(c4)c4ccccc4n5-c4cccc5c4oc4ccc(-c6ccccc6)cc45)n3)cc2)cc1 ; c1ccc(-c2cccc(-c3nc(-c4ccccc4)nc(-c4ccc5c(c4)c4ccccc4n5-c4cccc5c4oc4ccc(-c6ccccc6)cc45)n3)c2)cc1 ; c1ccc(-c2ccc3oc4c(-n5c6ccccc6c6cc(-c7nc(-c8ccccc8)nc(-c8ccc9c(c8)oc8ccccc89)n7)ccc65)cccc4c3c2)cc1 ; c1ccc(-c2ccc3oc4c(-n5c6ccccc6c6cc(-c7nc(-c8ccccc8)nc(-c8cccc9c8oc8ccccc89)n7)ccc65)cccc4c3c2)cc1]

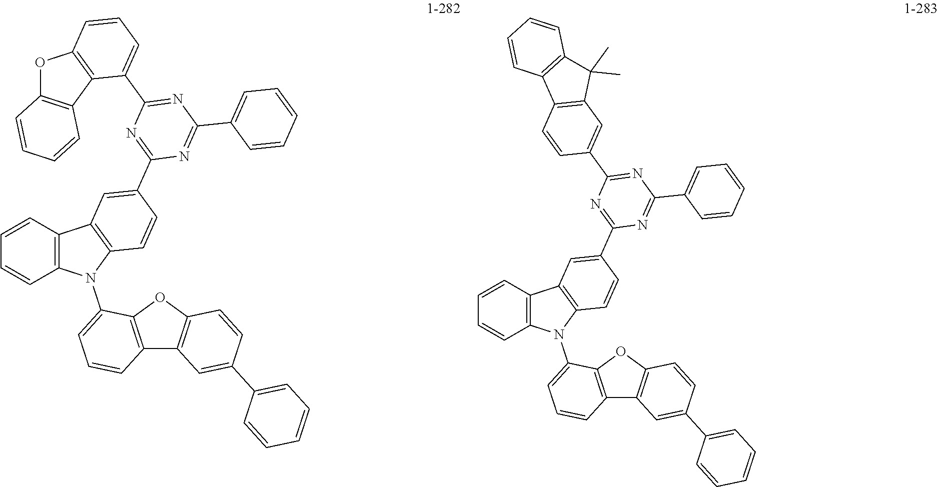 [SMILES: c1ccc(-c2ccc3oc4c(-n5c6ccccc6c6cc(-c7nc(-c8ccccc8)nc(-c8cccc9oc%10ccccc%10c89)n7)ccc65)cccc4c3c2)cc1 ; CC1(C)c2ccccc2-c2ccc(-c3nc(-c4ccccc4)nc(-c4ccc5c(c4)c4ccccc4n5-c4cccc5c4oc4ccc(-c6ccccc6)cc45)n3)cc21]

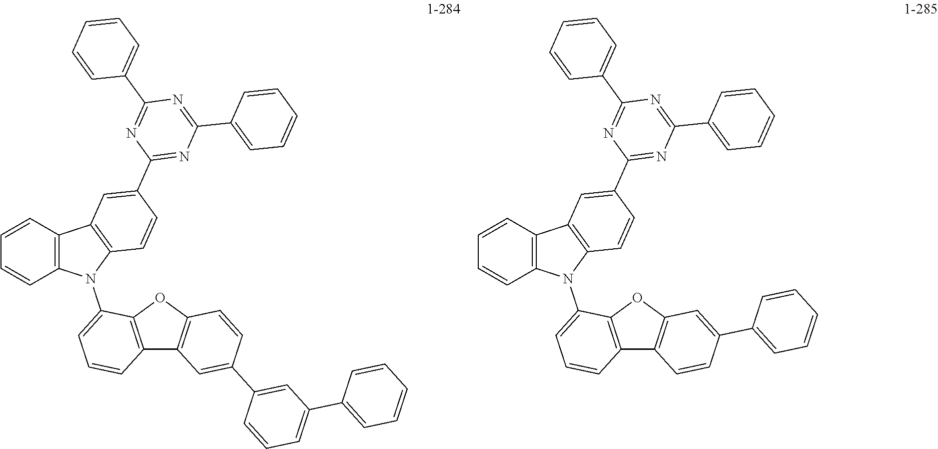 [SMILES: c1ccc(-c2cccc(-c3ccc4oc5c(-n6c7ccccc7c7cc(-c8nc(-c9ccccc9)nc(-c9ccccc9)n8)ccc76)cccc5c4c3)c2)cc1 ; c1ccc(-c2ccc3c(c2)oc2c(-n4c5ccccc5c5cc(-c6nc(-c7ccccc7)nc(-c7ccccc7)n6)ccc54)cccc23)cc1]

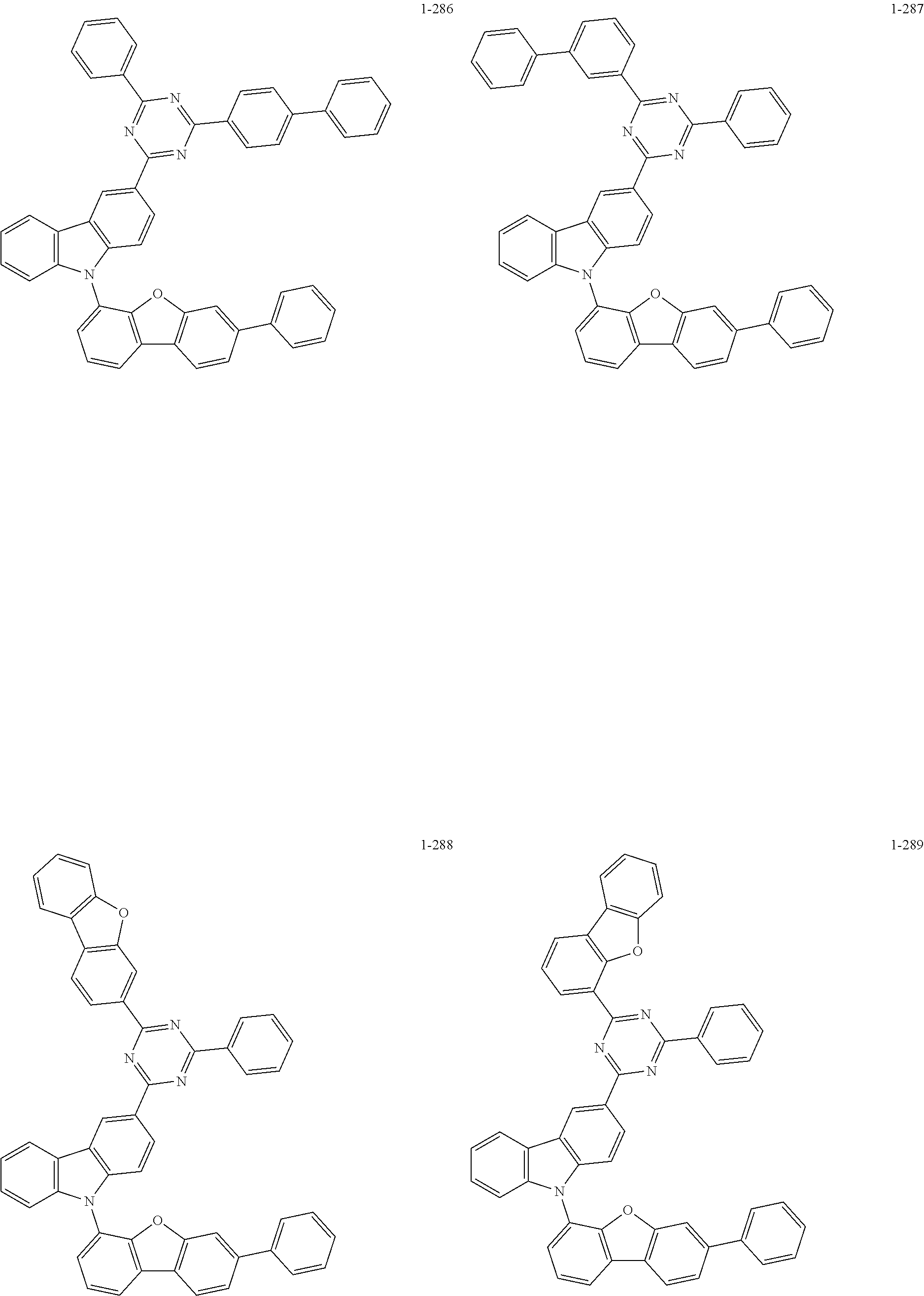 [SMILES: c1ccc(-c2ccc(-c3nc(-c4ccccc4)nc(-c4ccc5c(c4)c4ccccc4n5-c4cccc5c4oc4cc(-c6ccccc6)ccc45)n3)cc2)cc1 ; c1ccc(-c2cccc(-c3nc(-c4ccccc4)nc(-c4ccc5c(c4)c4ccccc4n5-c4cccc5c4oc4cc(-c6ccccc6)ccc45)n3)c2)cc1 ; c1ccc(-c2ccc3c(c2)oc2c(-n4c5ccccc5c5cc(-c6nc(-c7ccccc7)nc(-c7ccc8c(c7)oc7ccccc78)n6)ccc54)cccc23)cc1 ; c1ccc(-c2ccc3c(c2)oc2c(-n4c5ccccc5c5cc(-c6nc(-c7ccccc7)nc(-c7cccc8c7oc7ccccc78)n6)ccc54)cccc23)cc1]

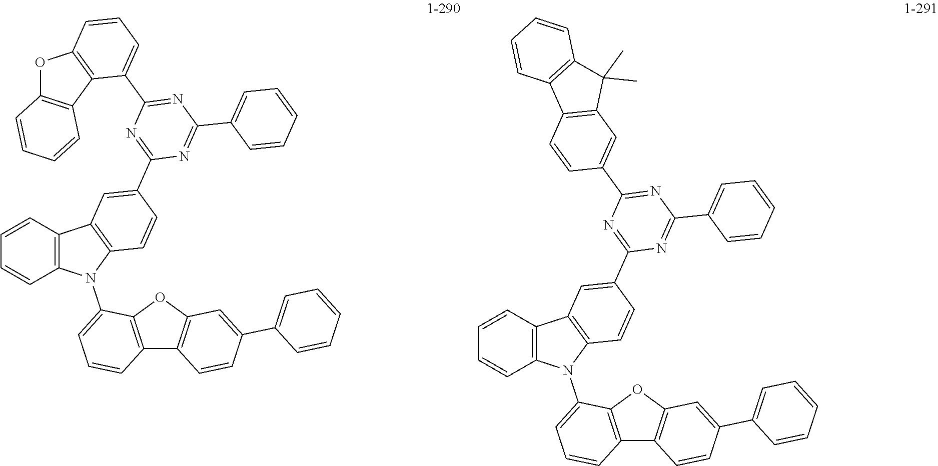 [SMILES: c1ccc(-c2ccc3c(c2)oc2c(-n4c5ccccc5c5cc(-c6nc(-c7ccccc7)nc(-c7cccc8oc9ccccc9c78)n6)ccc54)cccc23)cc1 ; CC1(C)c2ccccc2-c2ccc(-c3nc(-c4ccccc4)nc(-c4ccc5c(c4)c4ccccc4n5-c4cccc5c4oc4cc(-c6ccccc6)ccc45)n3)cc21]

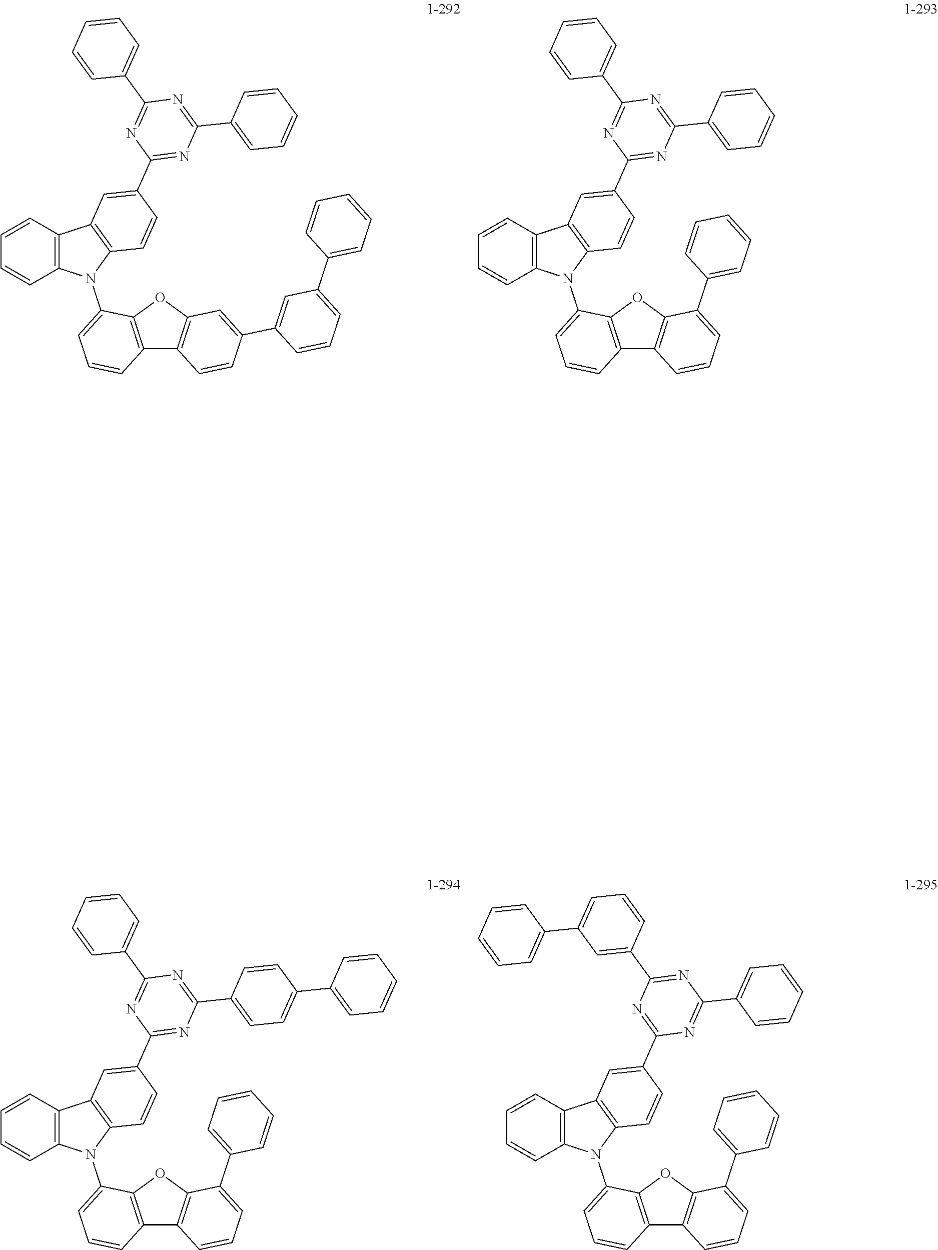 [SMILES: c1ccc(-c2cccc(-c3ccc4c(c3)oc3c(-n5c6ccccc6c6cc(-c7nc(-c8ccccc8)nc(-c8ccccc8)n7)ccc65)cccc34)c2)cc1 ; c1ccc(-c2nc(-c3ccccc3)nc(-c3ccc4c(c3)c3ccccc3n4-c3cccc4c3oc3c(-c5ccccc5)cccc34)n2)cc1 ; c1ccc(-c2ccc(-c3nc(-c4ccccc4)nc(-c4ccc5c(c4)c4ccccc4n5-c4cccc5c4oc4c(-c6ccccc6)cccc45)n3)cc2)cc1 ; c1ccc(-c2cccc(-c3nc(-c4ccccc4)nc(-c4ccc5c(c4)c4ccccc4n5-c4cccc5c4oc4c(-c6ccccc6)cccc45)n3)c2)cc1]

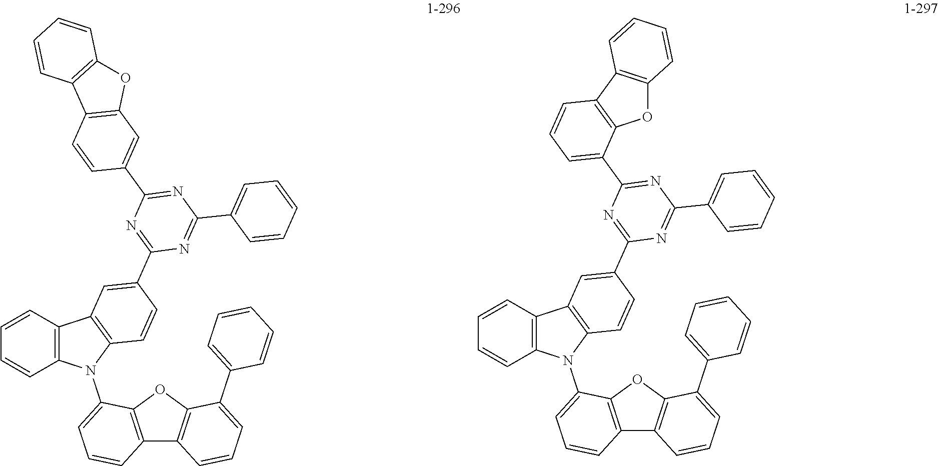 [SMILES: c1ccc(-c2nc(-c3ccc4c(c3)oc3ccccc34)nc(-c3ccc4c(c3)c3ccccc3n4-c3cccc4c3oc3c(-c5ccccc5)cccc34)n2)cc1 ; c1ccc(-c2nc(-c3ccc4c(c3)c3ccccc3n4-c3cccc4c3oc3c(-c5ccccc5)cccc34)nc(-c3cccc4c3oc3ccccc34)n2)cc1]

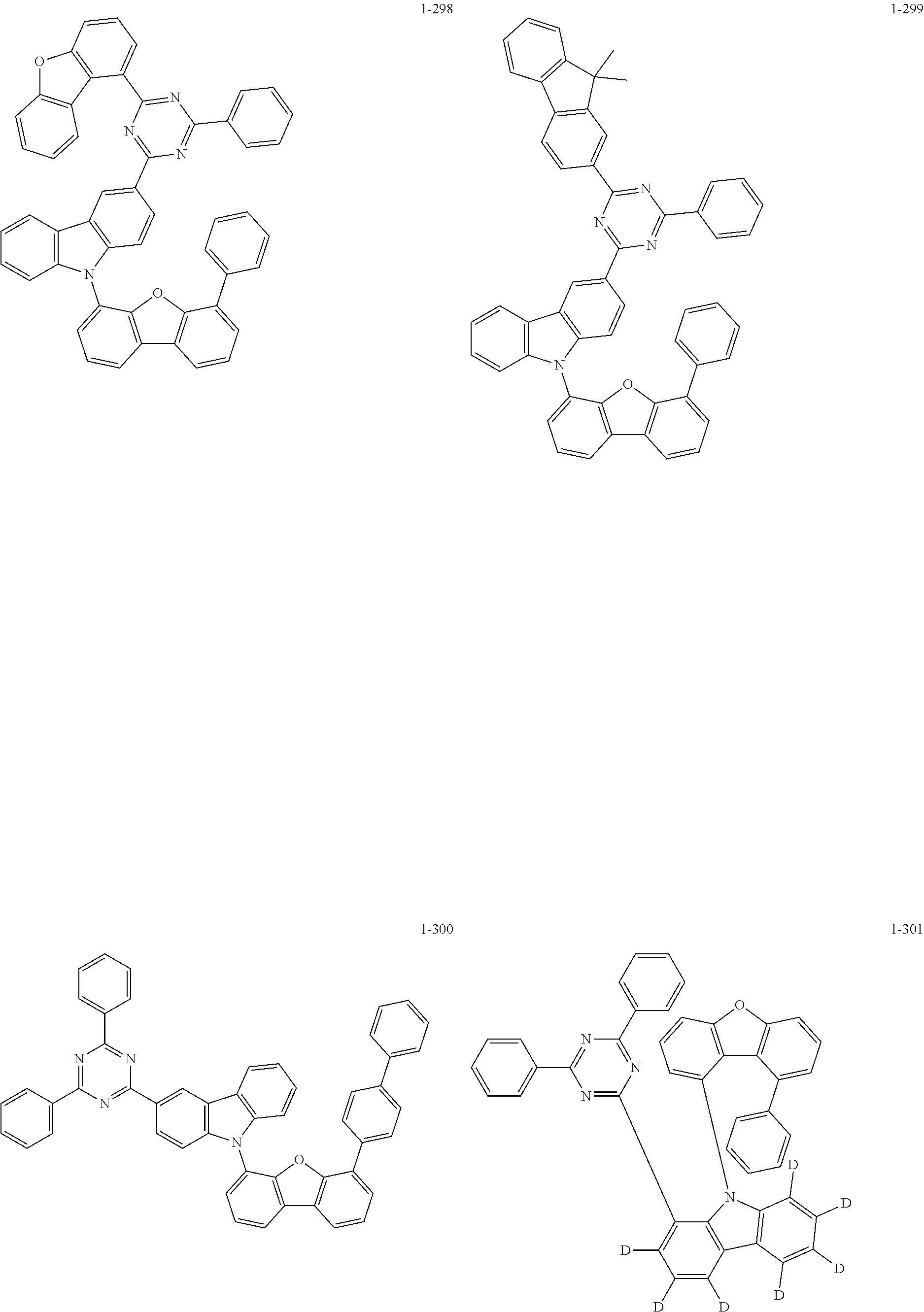 [SMILES: c1ccc(-c2nc(-c3ccc4c(c3)c3ccccc3n4-c3cccc4c3oc3c(-c5ccccc5)cccc34)nc(-c3cccc4oc5ccccc5c34)n2)cc1 ; CC1(C)c2ccccc2-c2ccc(-c3nc(-c4ccccc4)nc(-c4ccc5c(c4)c4ccccc4n5-c4cccc5c4oc4c(-c6ccccc6)cccc45)n3)cc21 ; c1ccc(-c2ccc(-c3cccc4c3oc3c(-n5c6ccccc6c6cc(-c7nc(-c8ccccc8)nc(-c8ccccc8)n7)ccc65)cccc34)cc2)cc1 ; [2H]c1c([2H])c([2H])c2c(c1[2H])c1c([2H])c([2H])c([2H])c(-c3nc(-c4ccccc4)nc(-c4ccccc4)n3)c1n2-c1cccc2oc3cccc(-c4ccccc4)c3c12]

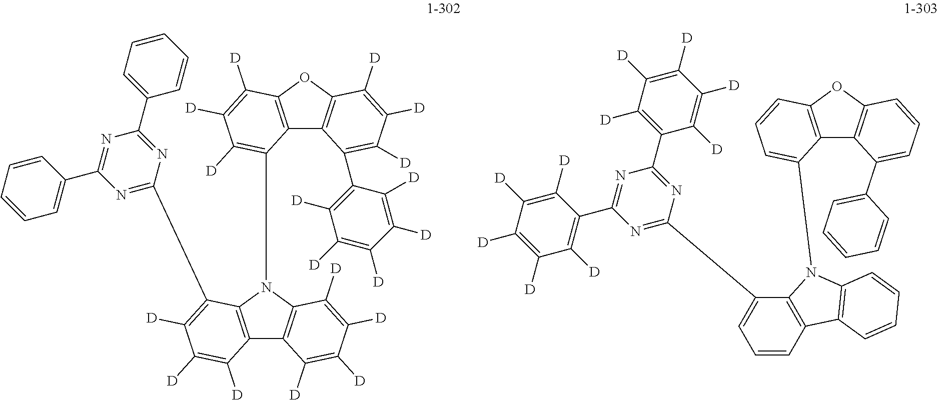 [SMILES: [2H]c1c([2H])c([2H])c(-c2c([2H])c([2H])c([2H])c3oc4c([2H])c([2H])c([2H])c(-n5c6c([2H])c([2H])c([2H])c([2H])c6c6c([2H])c([2H])c([2H])c(-c7nc(-c8ccccc8)nc(-c8ccccc8)n7)c65)c4c23)c([2H])c1[2H] ; [2H]c1c([2H])c([2H])c(-c2nc(-c3c([2H])c([2H])c([2H])c([2H])c3[2H])nc(-c3cccc4c5ccccc5n(-c5cccc6oc7cccc(-c8ccccc8)c7c56)c34)n2)c([2H])c1[2H]]

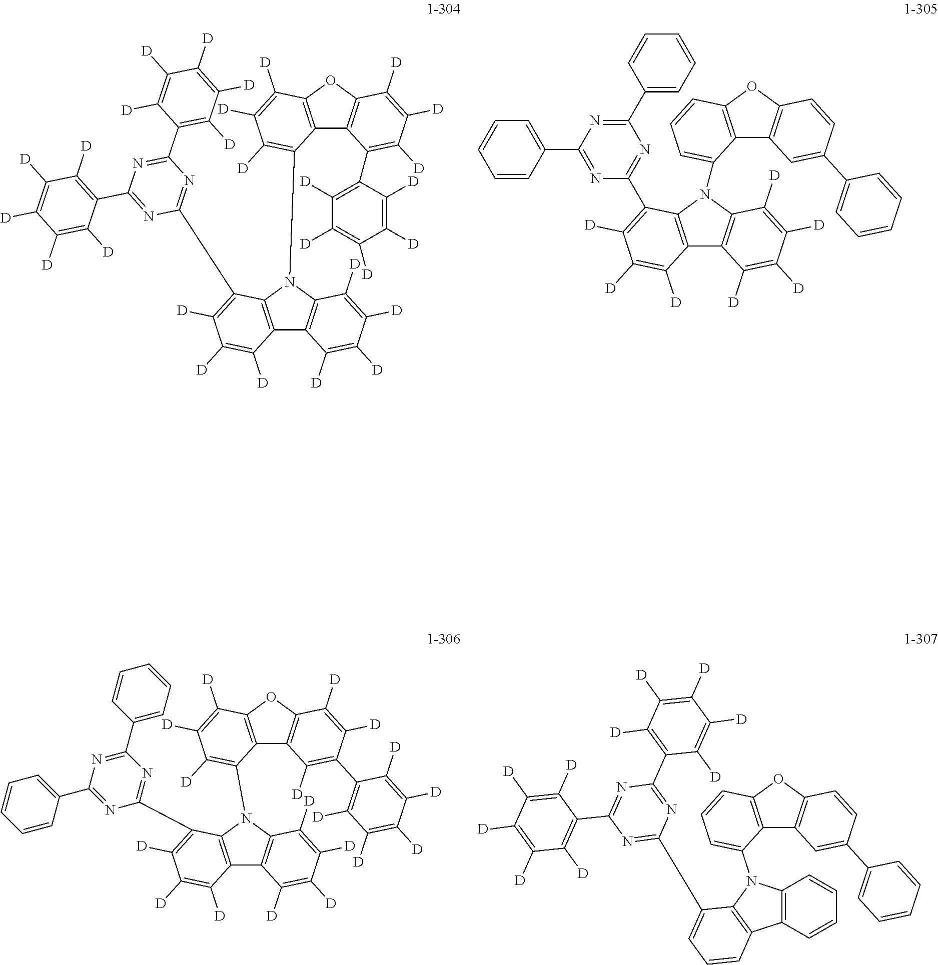

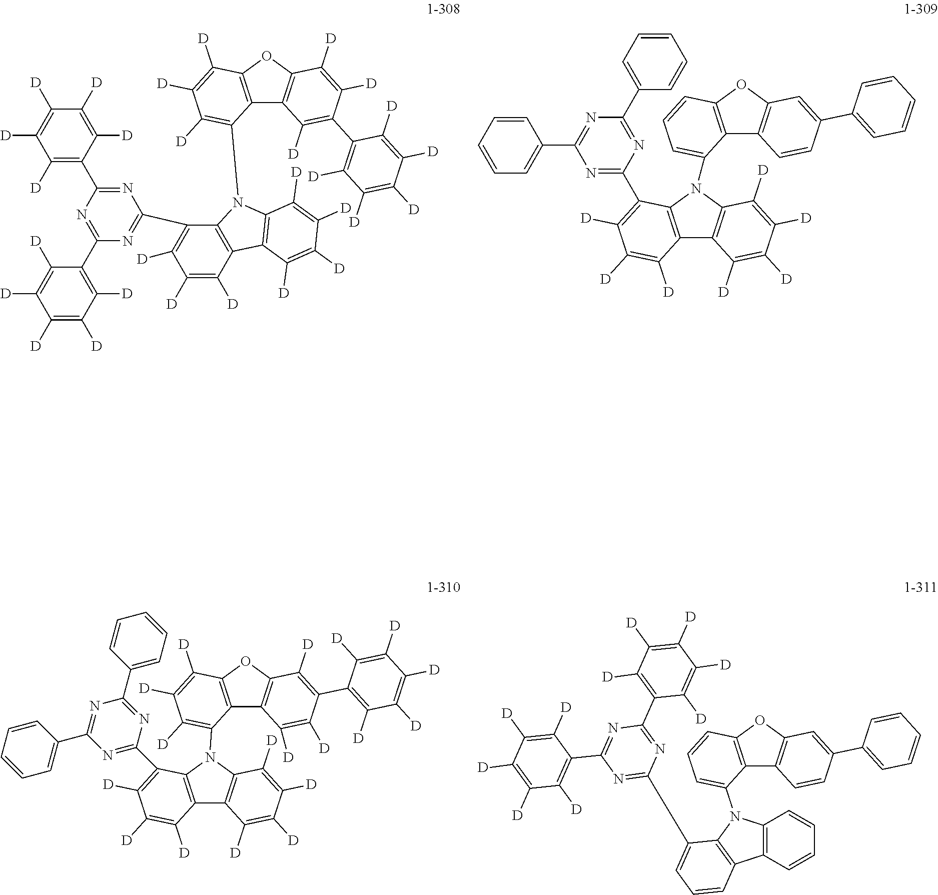

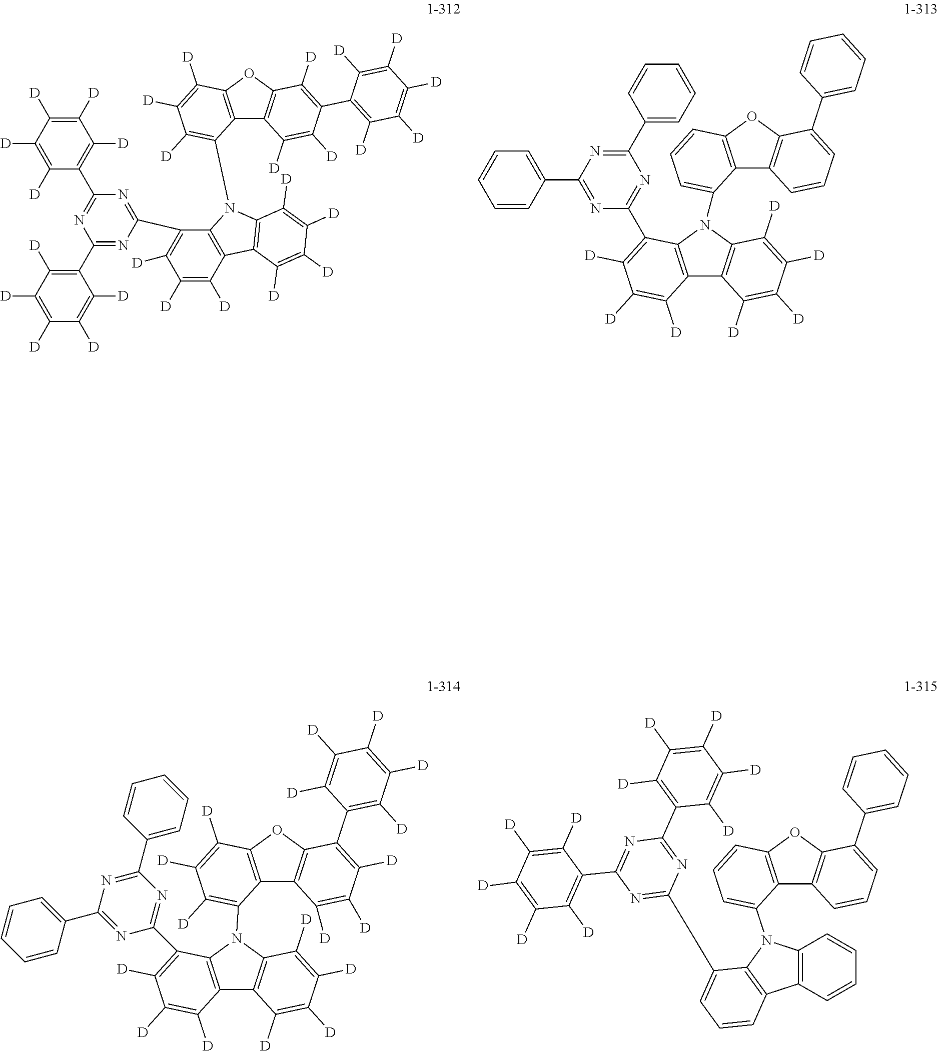

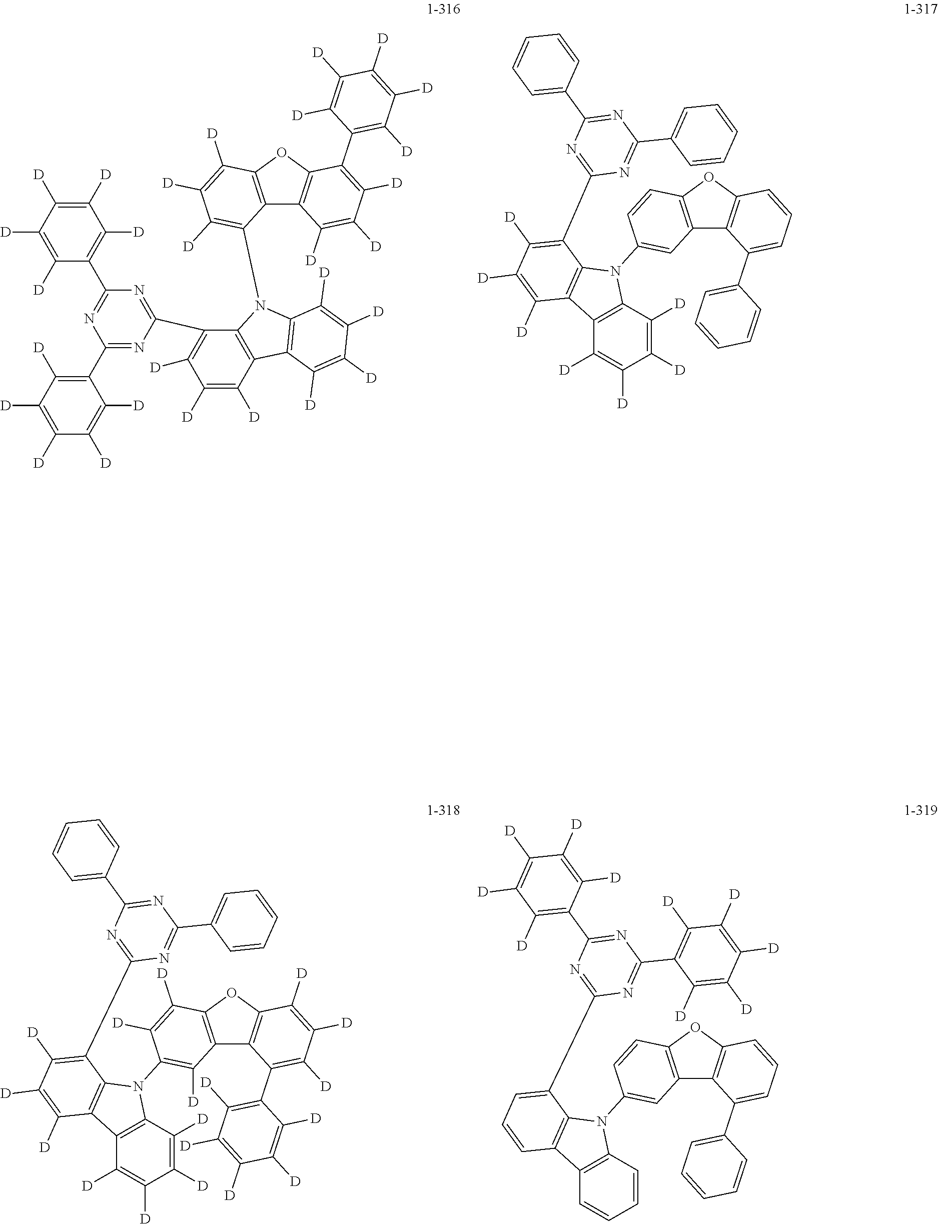

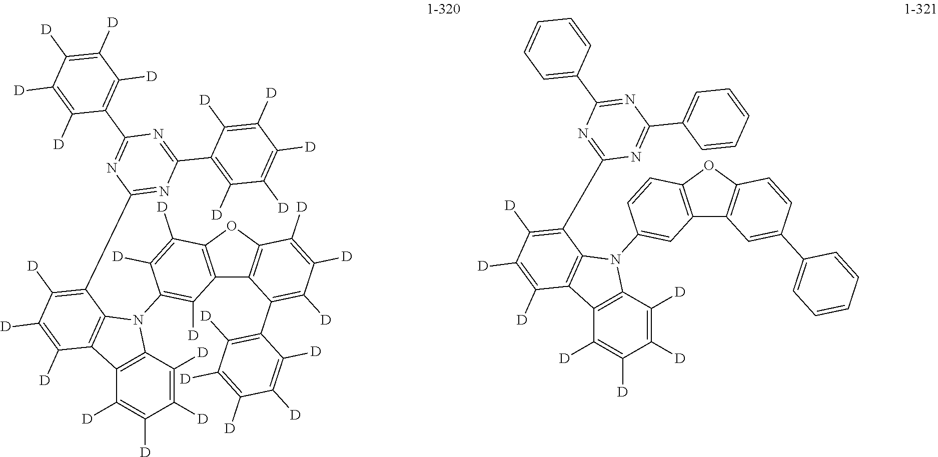 [SMILES: [2H]c1c([2H])c([2H])c(-c2nc(-c3c([2H])c([2H])c([2H])c([2H])c3[2H])nc(-c3c([2H])c([2H])c([2H])c4c5c([2H])c([2H])c([2H])c([2H])c5n(-c5c([2H])c([2H])c6oc7c([2H])c([2H])c([2H])c(-c8c([2H])c([2H])c([2H])c([2H])c8[2H])c7c6c5[2H])c34)n2)c([2H])c1[2H] ; [2H]c1c([2H])c([2H])c2c(c1[2H])c1c([2H])c([2H])c([2H])c(-c3nc(-c4ccccc4)nc(-c4ccccc4)n3)c1n2-c1ccc2oc3ccc(-c4ccccc4)cc3c2c1]

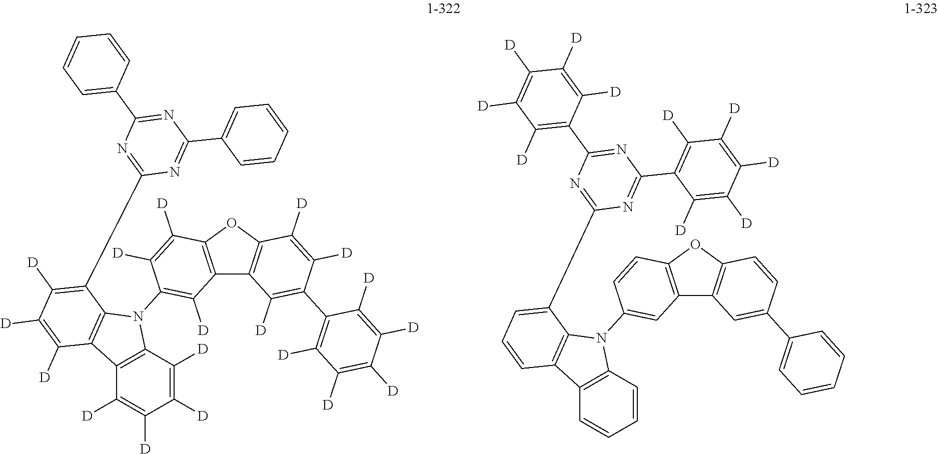 [SMILES: [2H]c1c([2H])c([2H])c(-c2c([2H])c([2H])c3oc4c([2H])c([2H])c(-n5c6c([2H])c([2H])c([2H])c([2H])c6c6c([2H])c([2H])c([2H])c(-c7nc(-c8ccccc8)nc(-c8ccccc8)n7)c65)c([2H])c4c3c2[2H])c([2H])c1[2H] ; [2H]c1c([2H])c([2H])c(-c2nc(-c3c([2H])c([2H])c([2H])c([2H])c3[2H])nc(-c3cccc4c5ccccc5n(-c5ccc6oc7ccc(-c8ccccc8)cc7c6c5)c34)n2)c([2H])c1[2H]]

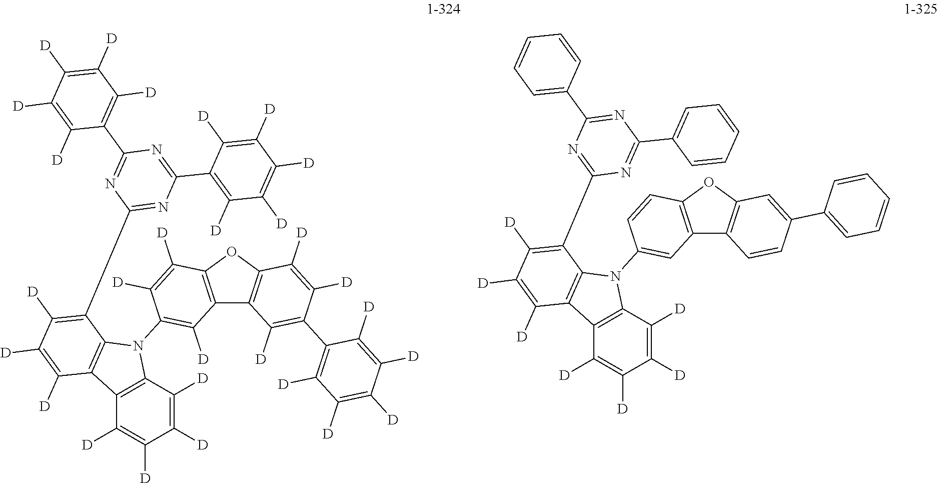 [SMILES: [2H]c1c([2H])c([2H])c(-c2nc(-c3c([2H])c([2H])c([2H])c([2H])c3[2H])nc(-c3c([2H])c([2H])c([2H])c4c5c([2H])c([2H])c([2H])c([2H])c5n(-c5c([2H])c([2H])c6oc7c([2H])c([2H])c(-c8c([2H])c([2H])c([2H])c([2H])c8[2H])c([2H])c7c6c5[2H])c34)n2)c([2H])c1[2H] ; [2H]c1c([2H])c([2H])c2c(c1[2H])c1c([2H])c([2H])c([2H])c(-c3nc(-c4ccccc4)nc(-c4ccccc4)n3)c1n2-c1ccc2oc3cc(-c4ccccc4)ccc3c2c1]

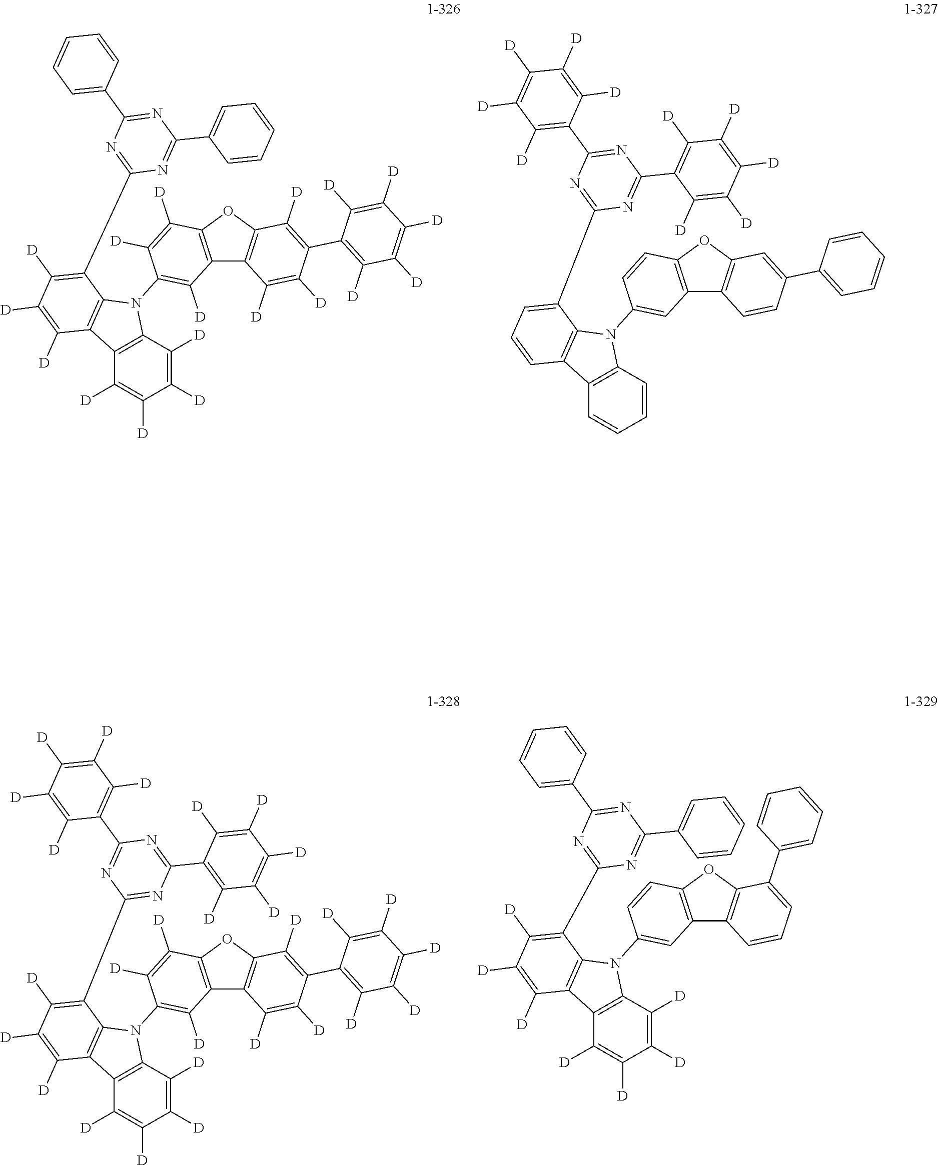

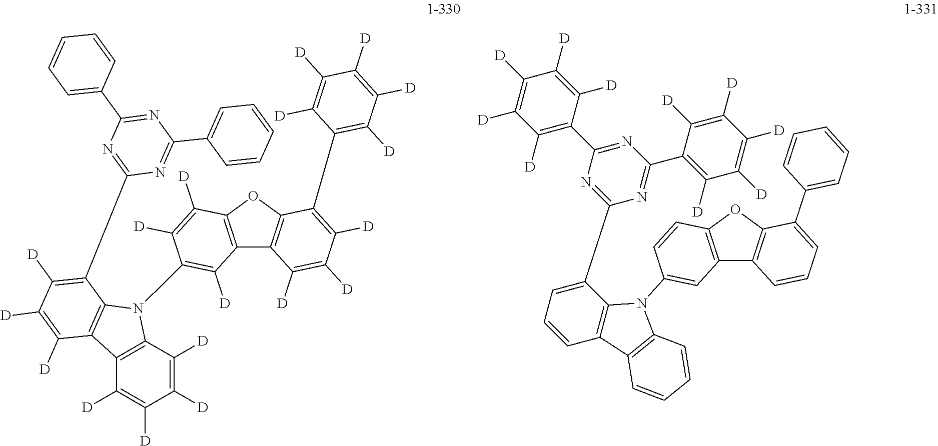 [SMILES: [2H]c1c([2H])c([2H])c(-c2c([2H])c([2H])c([2H])c3c2oc2c([2H])c([2H])c(-n4c5c([2H])c([2H])c([2H])c([2H])c5c5c([2H])c([2H])c([2H])c(-c6nc(-c7ccccc7)nc(-c7ccccc7)n6)c54)c([2H])c23)c([2H])c1[2H] ; [2H]c1c([2H])c([2H])c(-c2nc(-c3c([2H])c([2H])c([2H])c([2H])c3[2H])nc(-c3cccc4c5ccccc5n(-c5ccc6oc7c(-c8ccccc8)cccc7c6c5)c34)n2)c([2H])c1[2H]]

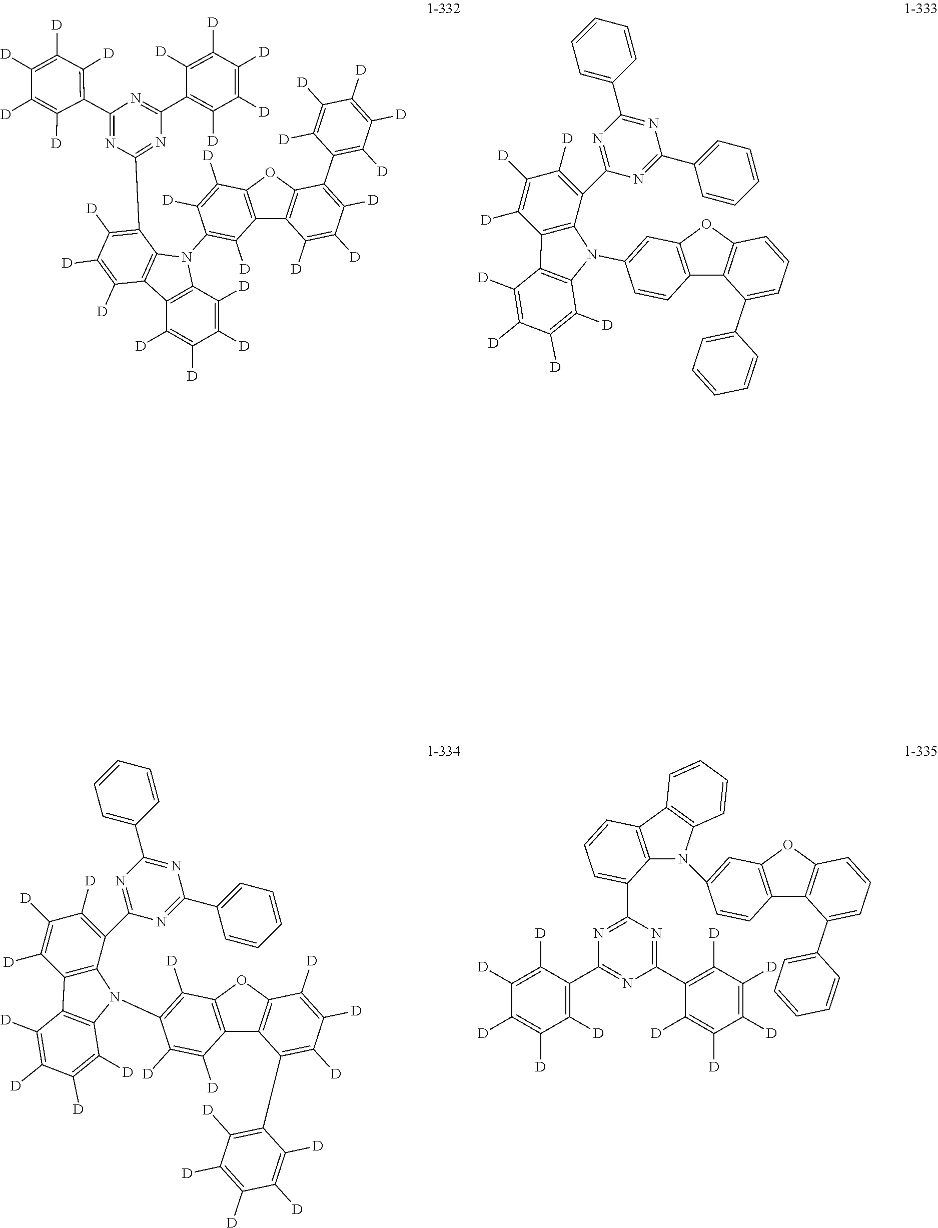

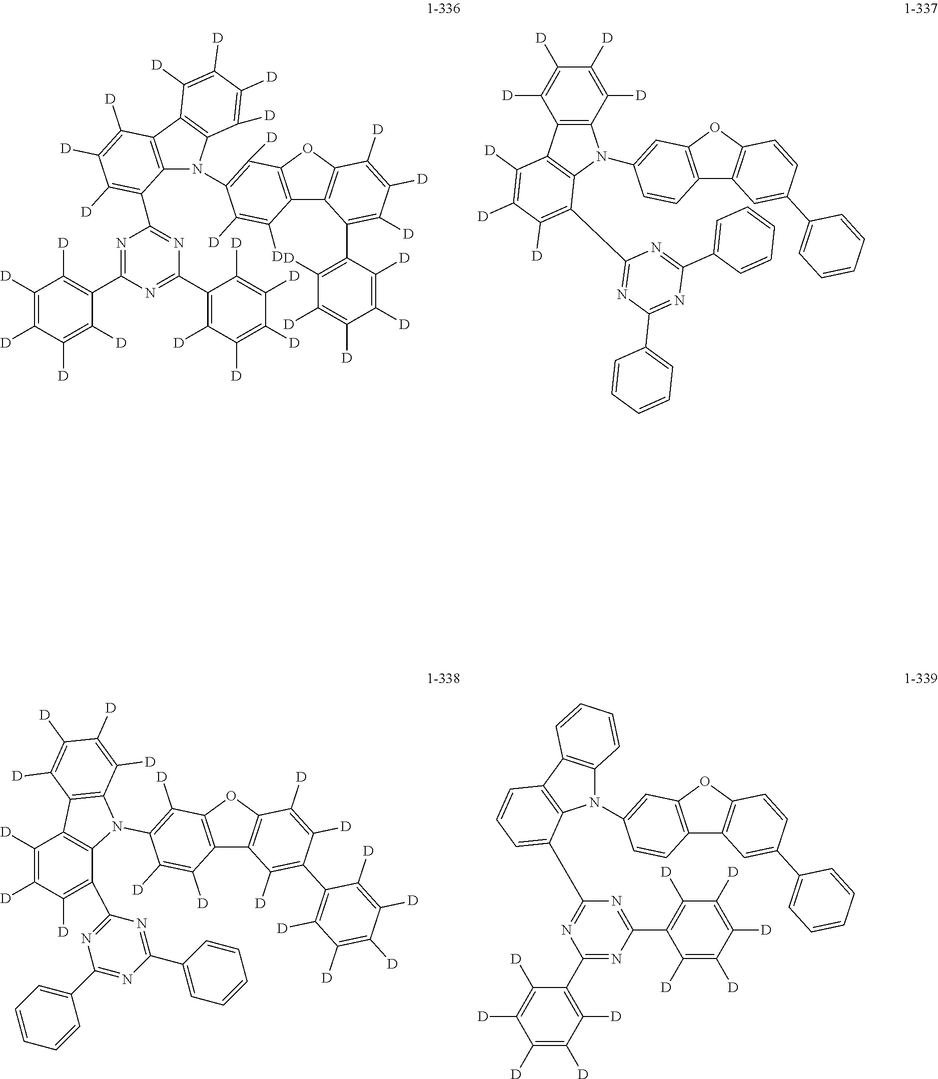

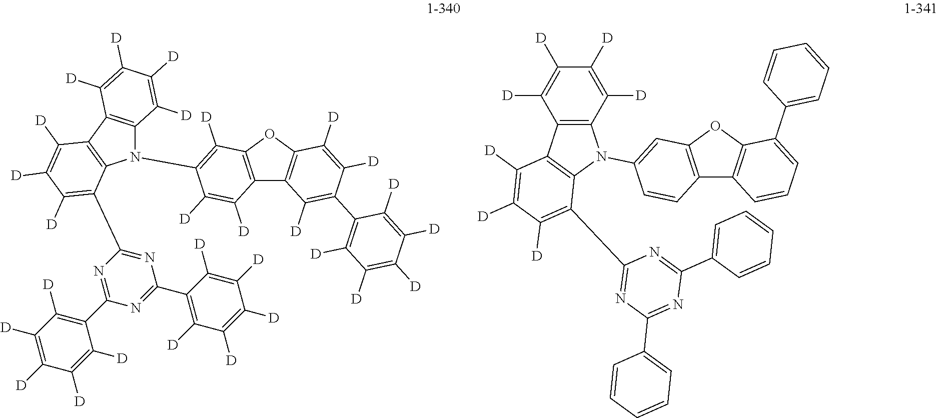 [SMILES: [2H]c1c([2H])c([2H])c(-c2nc(-c3c([2H])c([2H])c([2H])c([2H])c3[2H])nc(-c3c([2H])c([2H])c([2H])c4c5c([2H])c([2H])c([2H])c([2H])c5n(-c5c([2H])c([2H])c6c(oc7c([2H])c([2H])c(-c8c([2H])c([2H])c([2H])c([2H])c8[2H])c([2H])c76)c5[2H])c34)n2)c([2H])c1[2H] ; [2H]c1c([2H])c([2H])c2c(c1[2H])c1c([2H])c([2H])c([2H])c(-c3nc(-c4ccccc4)nc(-c4ccccc4)n3)c1n2-c1ccc2c(c1)oc1c(-c3ccccc3)cccc12]

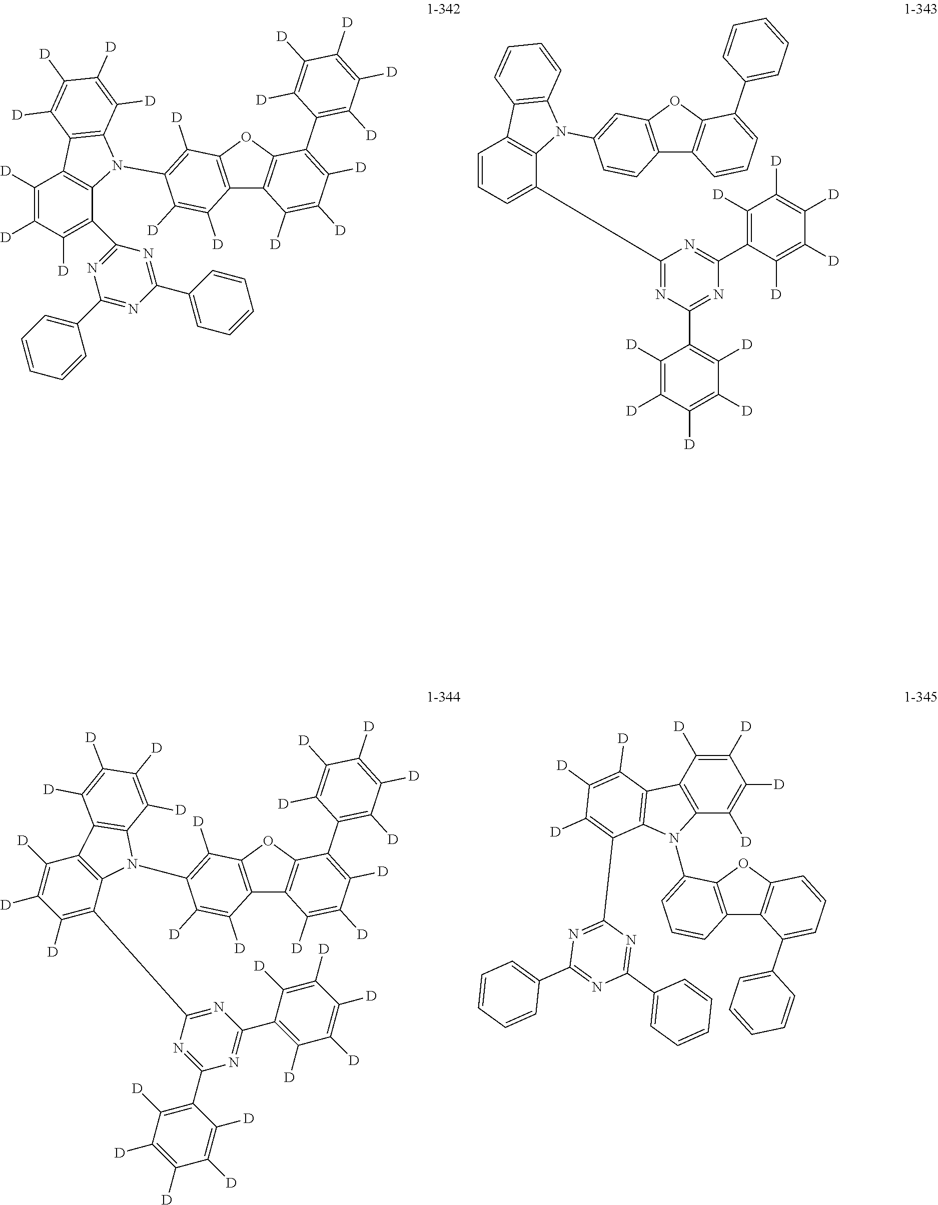

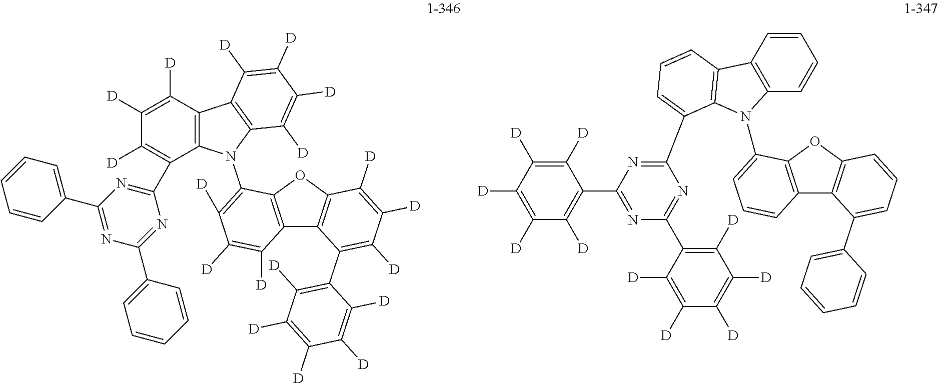 [SMILES: [2H]c1c([2H])c([2H])c(-c2c([2H])c([2H])c([2H])c3oc4c(-n5c6c([2H])c([2H])c([2H])c([2H])c6c6c([2H])c([2H])c([2H])c(-c7nc(-c8ccccc8)nc(-c8ccccc8)n7)c65)c([2H])c([2H])c([2H])c4c23)c([2H])c1[2H] ; [2H]c1c([2H])c([2H])c(-c2nc(-c3c([2H])c([2H])c([2H])c([2H])c3[2H])nc(-c3cccc4c5ccccc5n(-c5cccc6c5oc5cccc(-c7ccccc7)c56)c34)n2)c([2H])c1[2H]]

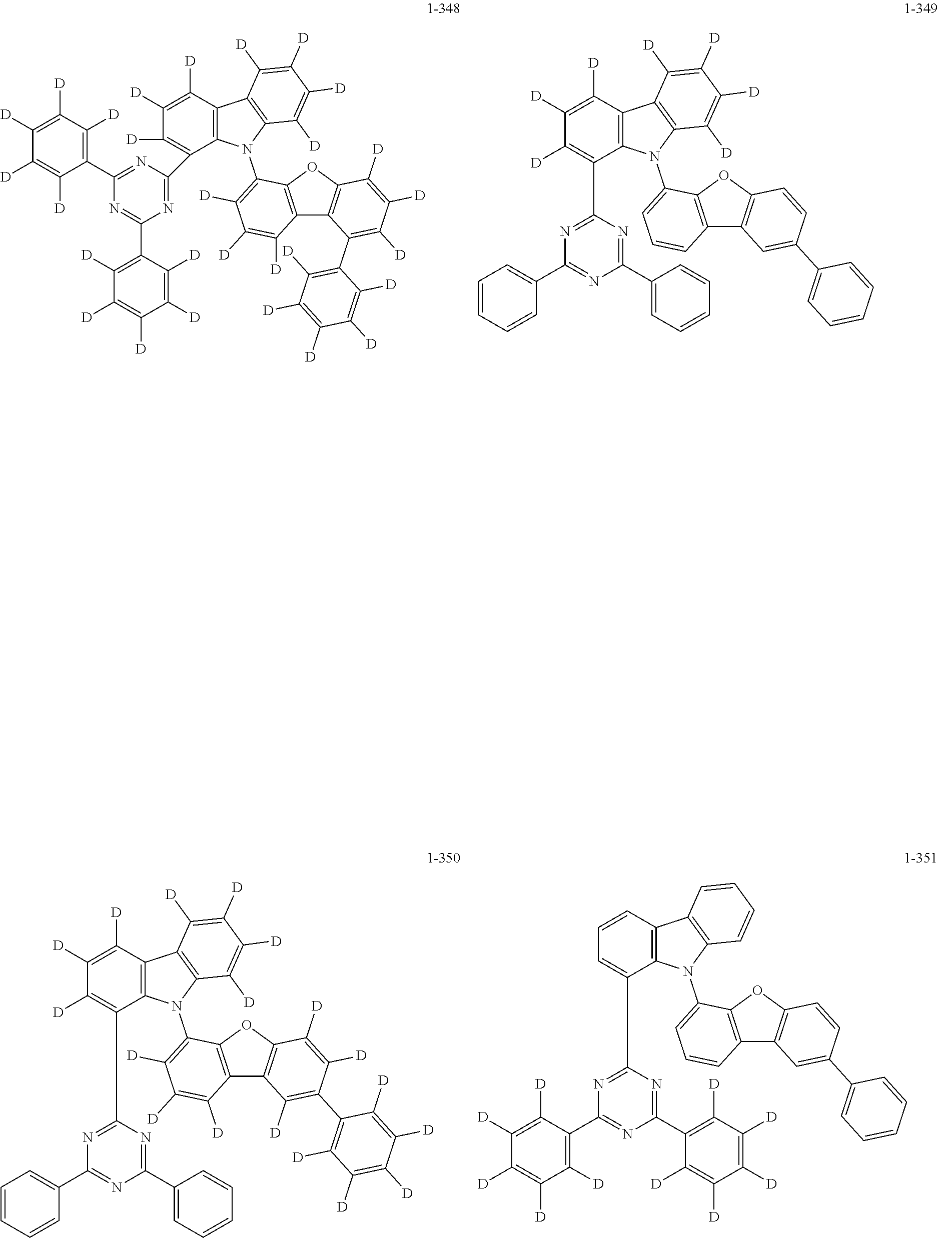

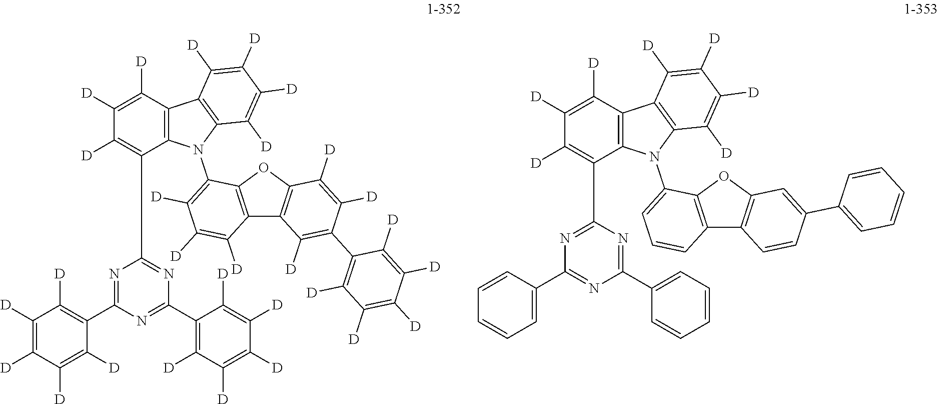 [SMILES: [2H]c1c([2H])c([2H])c(-c2nc(-c3c([2H])c([2H])c([2H])c([2H])c3[2H])nc(-c3c([2H])c([2H])c([2H])c4c5c([2H])c([2H])c([2H])c([2H])c5n(-c5c([2H])c([2H])c([2H])c6c5oc5c([2H])c([2H])c(-c7c([2H])c([2H])c([2H])c([2H])c7[2H])c([2H])c56)c34)n2)c([2H])c1[2H] ; [2H]c1c([2H])c([2H])c2c(c1[2H])c1c([2H])c([2H])c([2H])c(-c3nc(-c4ccccc4)nc(-c4ccccc4)n3)c1n2-c1cccc2c1oc1cc(-c3ccccc3)ccc12]

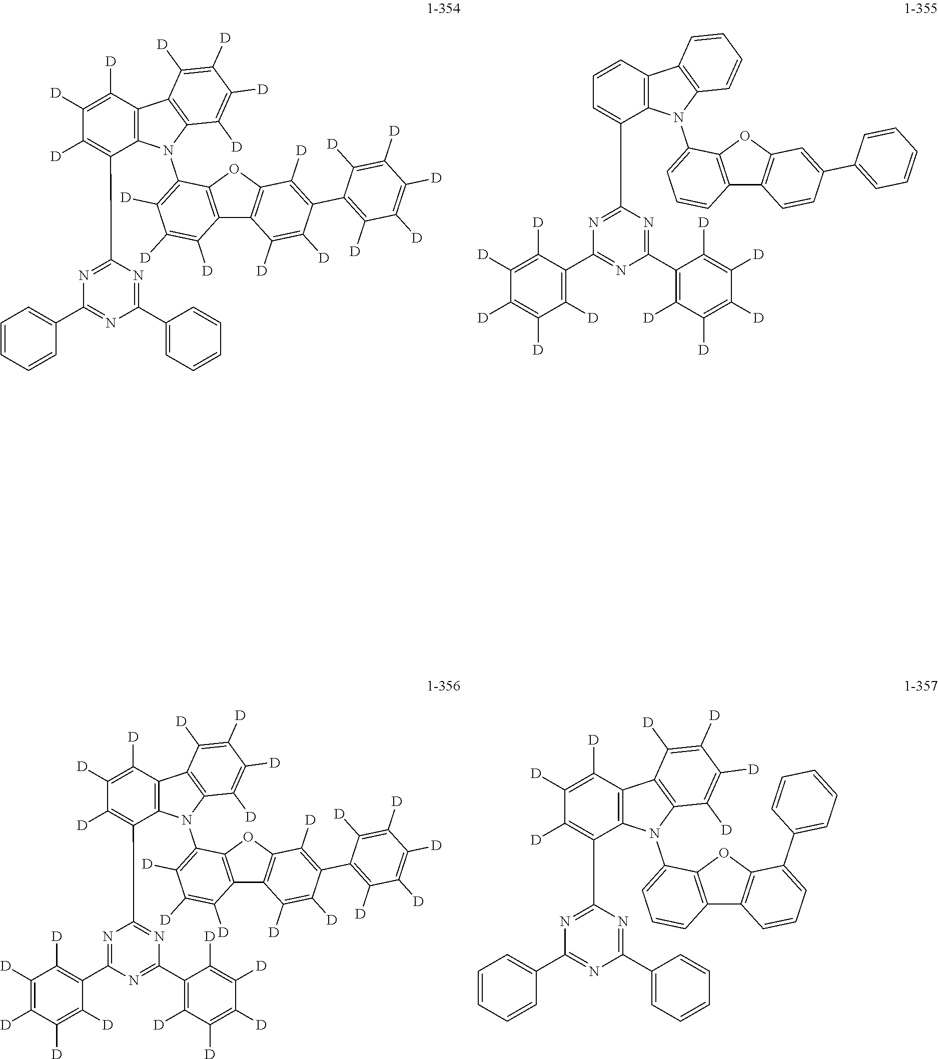

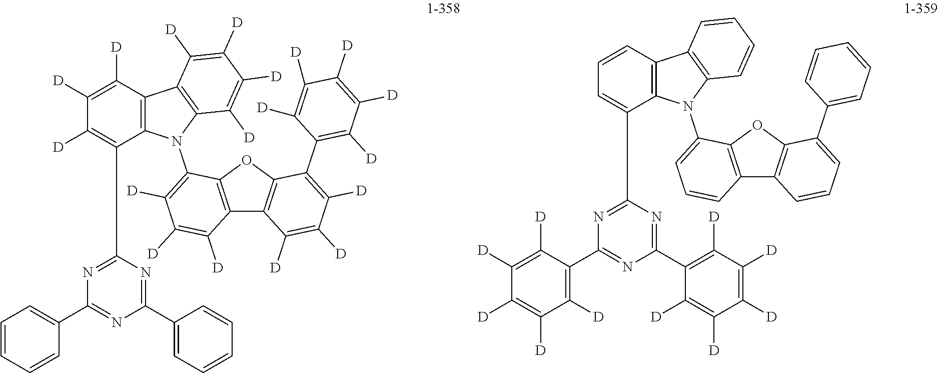 [SMILES: [2H]c1c([2H])c([2H])c(-c2c([2H])c([2H])c([2H])c3c2oc2c(-n4c5c([2H])c([2H])c([2H])c([2H])c5c5c([2H])c([2H])c([2H])c(-c6nc(-c7ccccc7)nc(-c7ccccc7)n6)c54)c([2H])c([2H])c([2H])c23)c([2H])c1[2H] ; [2H]c1c([2H])c([2H])c(-c2nc(-c3c([2H])c([2H])c([2H])c([2H])c3[2H])nc(-c3cccc4c5ccccc5n(-c5cccc6c5oc5c(-c7ccccc7)cccc56)c34)n2)c([2H])c1[2H]]

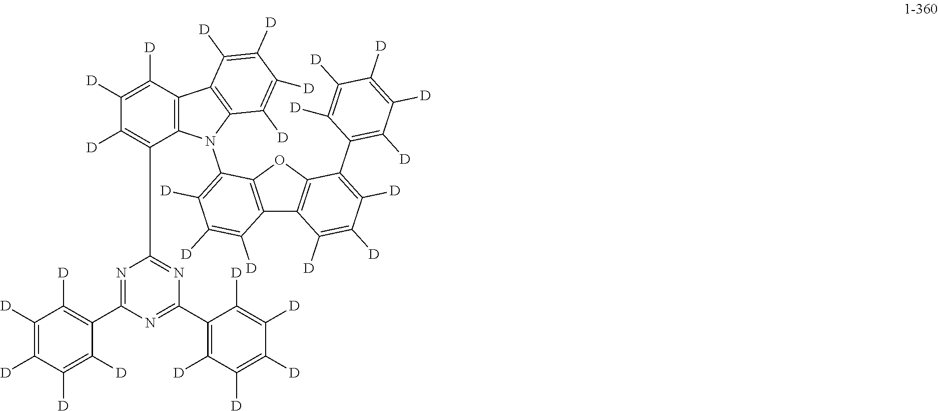 [SMILES: [2H]c1c([2H])c([2H])c(-c2nc(-c3c([2H])c([2H])c([2H])c([2H])c3[2H])nc(-c3c([2H])c([2H])c([2H])c4c5c([2H])c([2H])c([2H])c([2H])c5n(-c5c([2H])c([2H])c([2H])c6c5oc5c(-c7c([2H])c([2H])c([2H])c([2H])c7[2H])c([2H])c([2H])c([2H])c56)c34)n2)c([2H])c1[2H]]

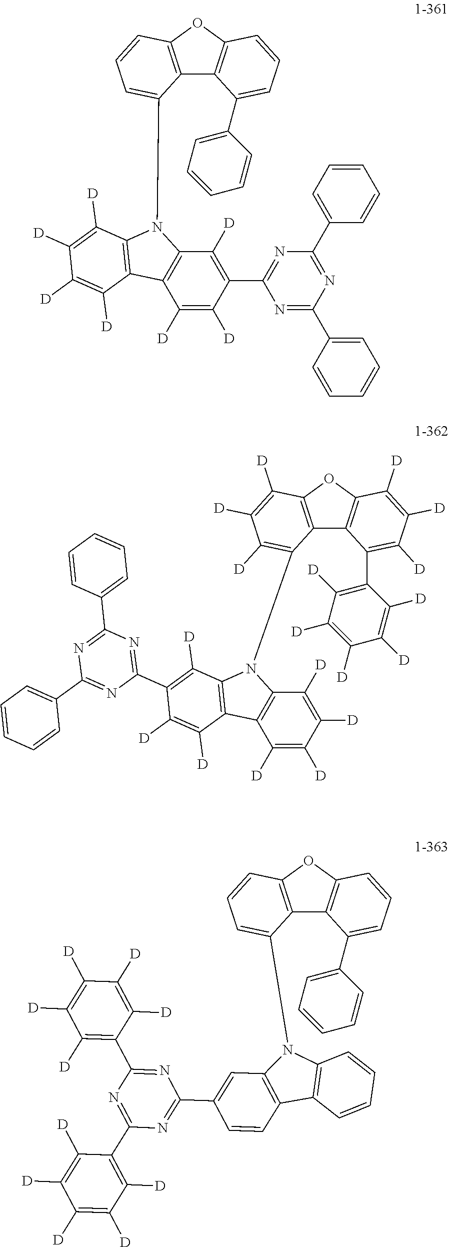 [SMILES: [2H]c1c([2H])c([2H])c2c(c1[2H])c1c([2H])c([2H])c(-c3nc(-c4ccccc4)nc(-c4ccccc4)n3)c([2H])c1n2-c1cccc2oc3cccc(-c4ccccc4)c3c12 ; [2H]c1c([2H])c([2H])c(-c2c([2H])c([2H])c([2H])c3oc4c([2H])c([2H])c([2H])c(-n5c6c([2H])c([2H])c([2H])c([2H])c6c6c([2H])c([2H])c(-c7nc(-c8ccccc8)nc(-c8ccccc8)n7)c([2H])c65)c4c23)c([2H])c1[2H] ; [2H]c1c([2H])c([2H])c(-c2nc(-c3ccc4c5ccccc5n(-c5cccc6oc7cccc(-c8ccccc8)c7c56)c4c3)nc(-c3c([2H])c([2H])c([2H])c([2H])c3[2H])n2)c([2H])c1[2H]]

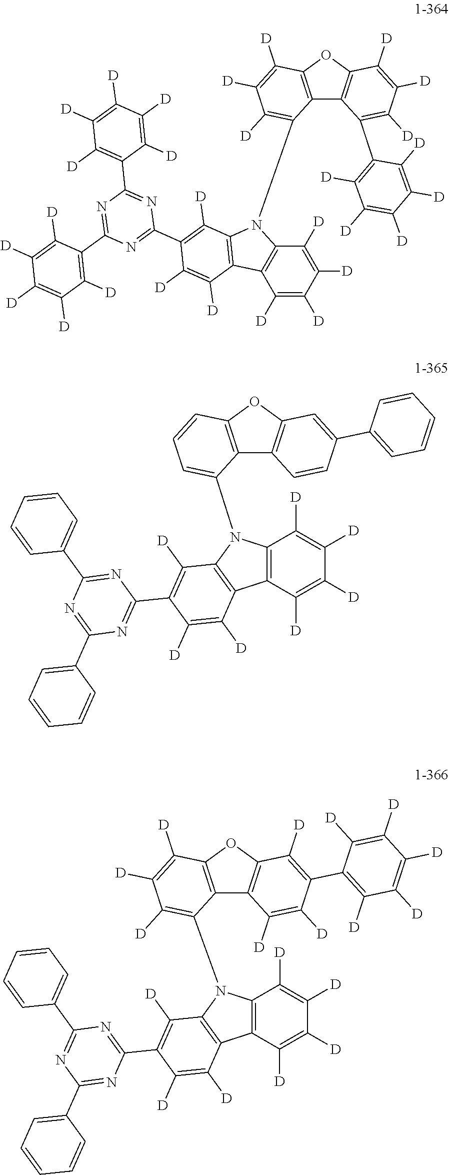

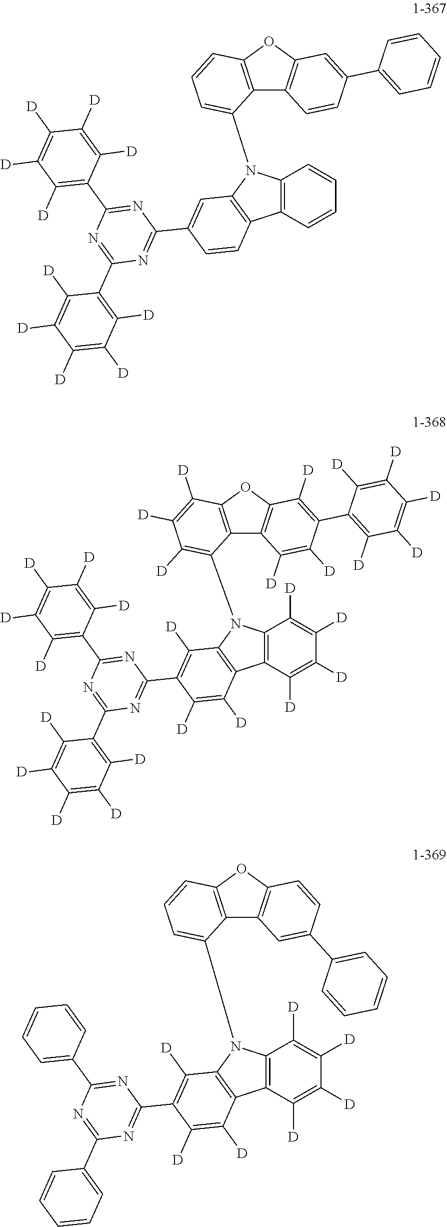

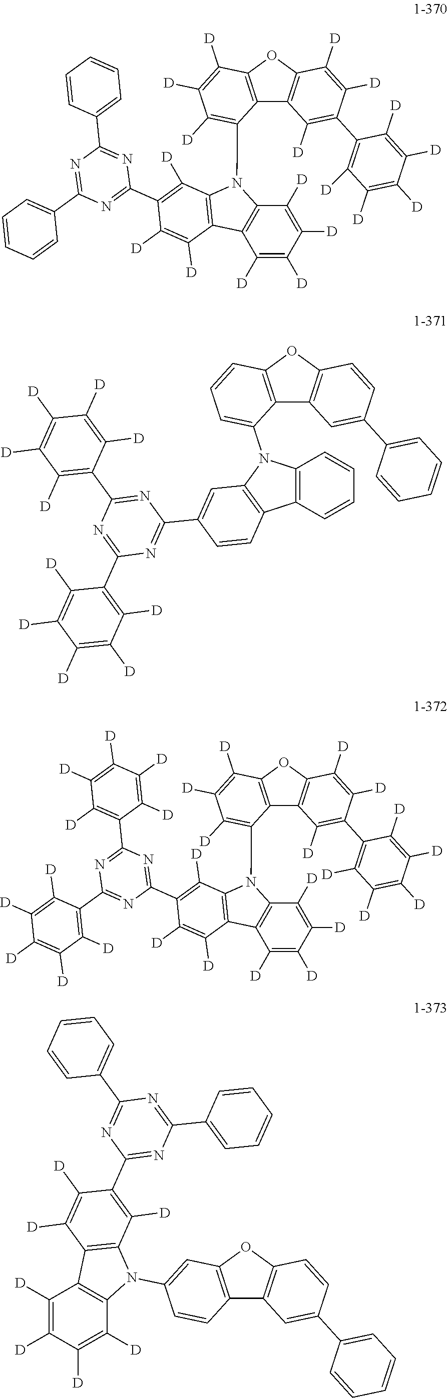

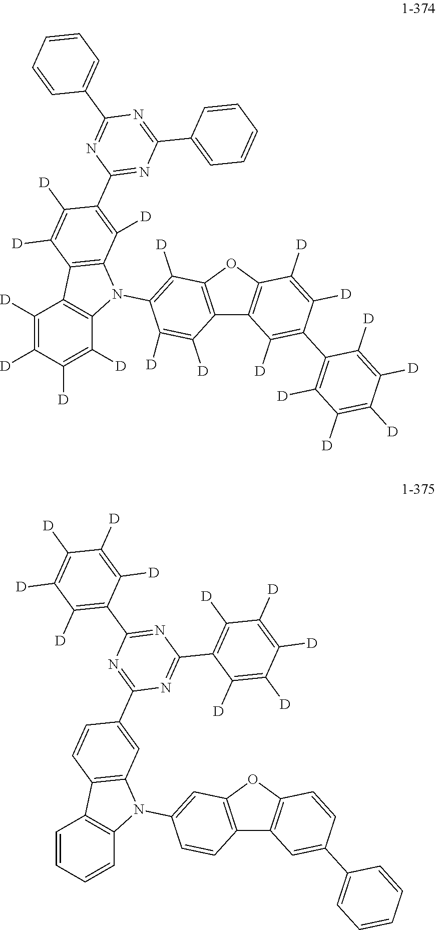 [SMILES: [2H]c1c([2H])c([2H])c(-c2c([2H])c([2H])c3oc4c([2H])c(-n5c6c([2H])c([2H])c([2H])c([2H])c6c6c([2H])c([2H])c(-c7nc(-c8ccccc8)nc(-c8ccccc8)n7)c([2H])c65)c([2H])c([2H])c4c3c2[2H])c([2H])c1[2H] ; [2H]c1c([2H])c([2H])c(-c2nc(-c3ccc4c5ccccc5n(-c5ccc6c(c5)oc5ccc(-c7ccccc7)cc56)c4c3)nc(-c3c([2H])c([2H])c([2H])c([2H])c3[2H])n2)c([2H])c1[2H]]

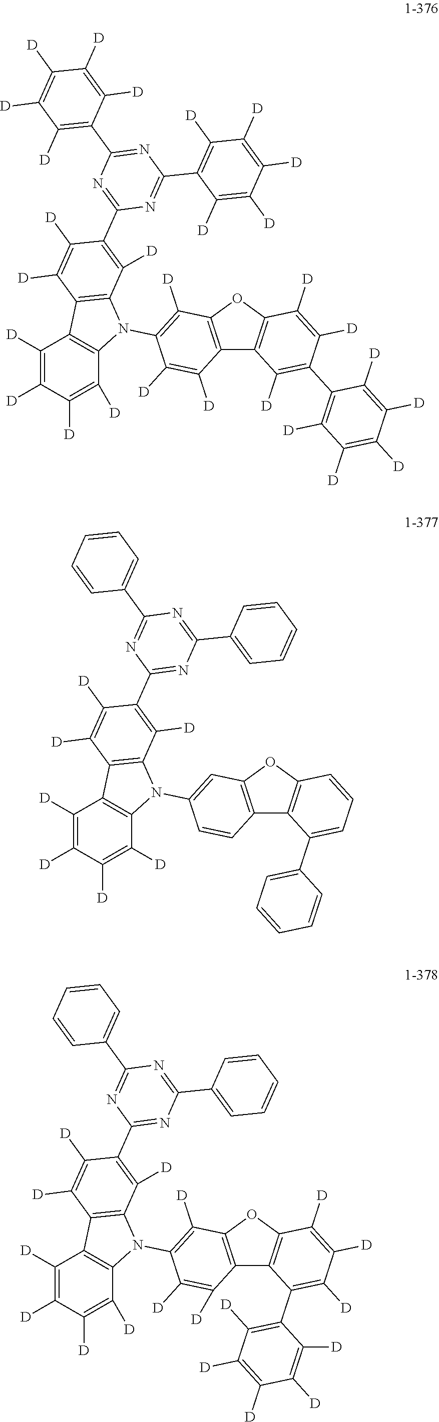

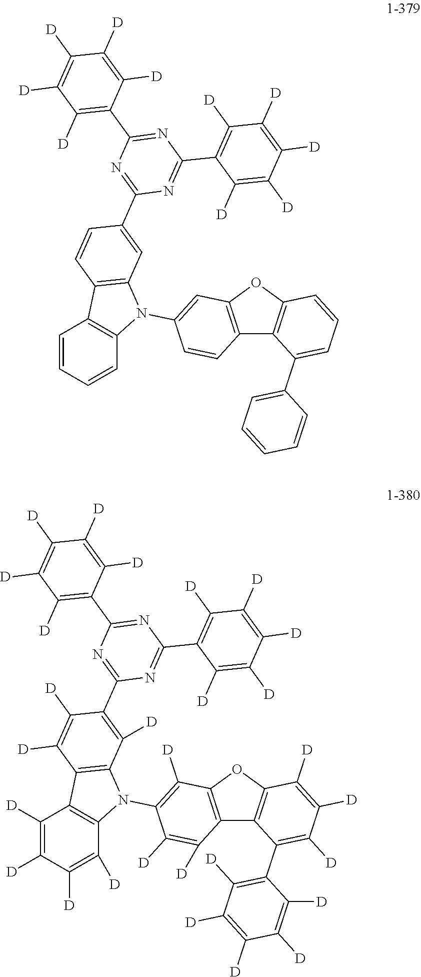 [SMILES: [2H]c1c([2H])c([2H])c(-c2nc(-c3ccc4c5ccccc5n(-c5ccc6c(c5)oc5cccc(-c7ccccc7)c56)c4c3)nc(-c3c([2H])c([2H])c([2H])c([2H])c3[2H])n2)c([2H])c1[2H] ; [2H]c1c([2H])c([2H])c(-c2nc(-c3c([2H])c([2H])c([2H])c([2H])c3[2H])nc(-c3c([2H])c([2H])c4c5c([2H])c([2H])c([2H])c([2H])c5n(-c5c([2H])c([2H])c6c(oc7c([2H])c([2H])c([2H])c(-c8c([2H])c([2H])c([2H])c([2H])c8[2H])c76)c5[2H])c4c3[2H])n2)c([2H])c1[2H]]

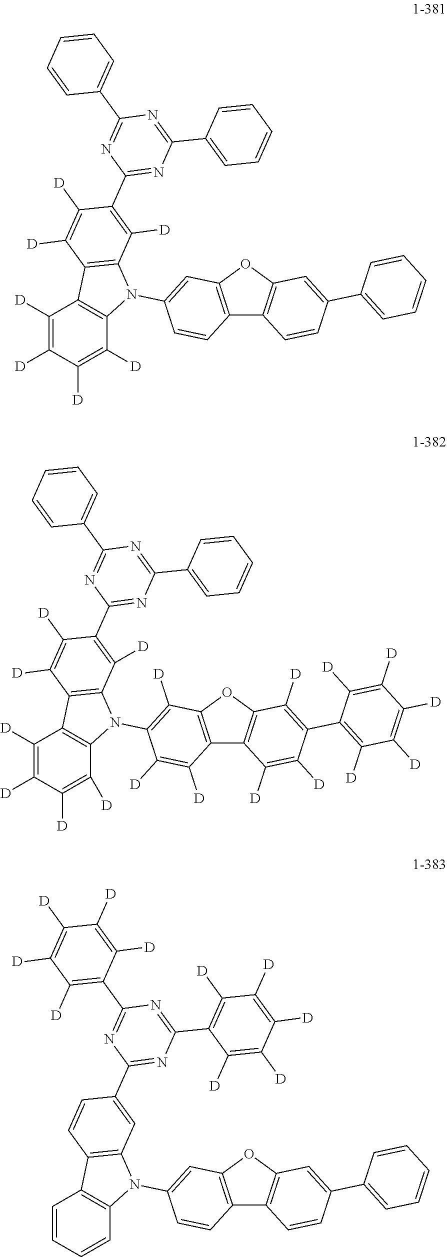 [SMILES: [2H]c1c([2H])c([2H])c2c(c1[2H])c1c([2H])c([2H])c(-c3nc(-c4ccccc4)nc(-c4ccccc4)n3)c([2H])c1n2-c1ccc2c(c1)oc1cc(-c3ccccc3)ccc12 ; [2H]c1c([2H])c([2H])c(-c2c([2H])c([2H])c3c(oc4c([2H])c(-n5c6c([2H])c([2H])c([2H])c([2H])c6c6c([2H])c([2H])c(-c7nc(-c8ccccc8)nc(-c8ccccc8)n7)c([2H])c65)c([2H])c([2H])c43)c2[2H])c([2H])c1[2H] ; [2H]c1c([2H])c([2H])c(-c2nc(-c3ccc4c5ccccc5n(-c5ccc6c(c5)oc5cc(-c7ccccc7)ccc56)c4c3)nc(-c3c([2H])c([2H])c([2H])c([2H])c3[2H])n2)c([2H])c1[2H]]

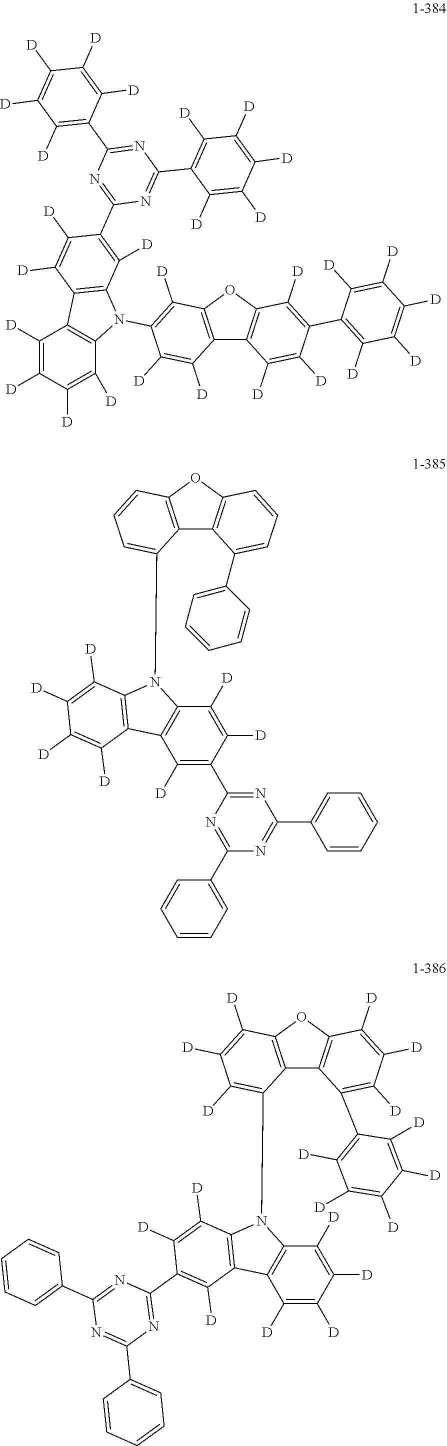

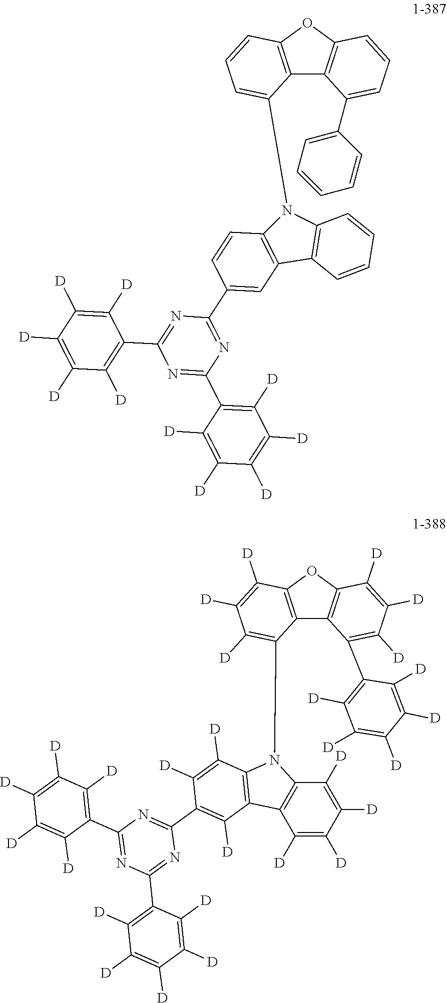 [SMILES: [2H]c1c([2H])c([2H])c(-c2nc(-c3ccc4c(c3)c3ccccc3n4-c3cccc4oc5cccc(-c6ccccc6)c5c34)nc(-c3c([2H])c([2H])c([2H])c([2H])c3[2H])n2)c([2H])c1[2H] ; [2H]c1c([2H])c([2H])c(-c2nc(-c3c([2H])c([2H])c([2H])c([2H])c3[2H])nc(-c3c([2H])c([2H])c4c(c3[2H])c3c([2H])c([2H])c([2H])c([2H])c3n4-c3c([2H])c([2H])c([2H])c4oc5c([2H])c([2H])c([2H])c(-c6c([2H])c([2H])c([2H])c([2H])c6[2H])c5c34)n2)c([2H])c1[2H]]

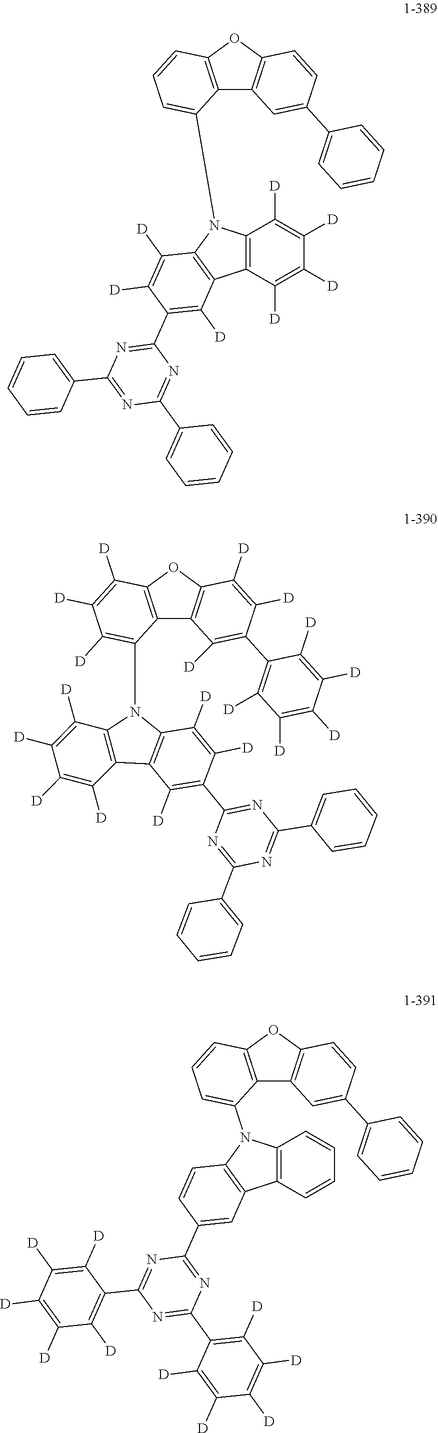 [SMILES: [2H]c1c([2H])c([2H])c2c(c1[2H])c1c([2H])c(-c3nc(-c4ccccc4)nc(-c4ccccc4)n3)c([2H])c([2H])c1n2-c1cccc2oc3ccc(-c4ccccc4)cc3c12 ; [2H]c1c([2H])c([2H])c(-c2c([2H])c([2H])c3oc4c([2H])c([2H])c([2H])c(-n5c6c([2H])c([2H])c([2H])c([2H])c6c6c([2H])c(-c7nc(-c8ccccc8)nc(-c8ccccc8)n7)c([2H])c([2H])c65)c4c3c2[2H])c([2H])c1[2H] ; [2H]c1c([2H])c([2H])c(-c2nc(-c3ccc4c(c3)c3ccccc3n4-c3cccc4oc5ccc(-c6ccccc6)cc5c34)nc(-c3c([2H])c([2H])c([2H])c([2H])c3[2H])n2)c([2H])c1[2H]]

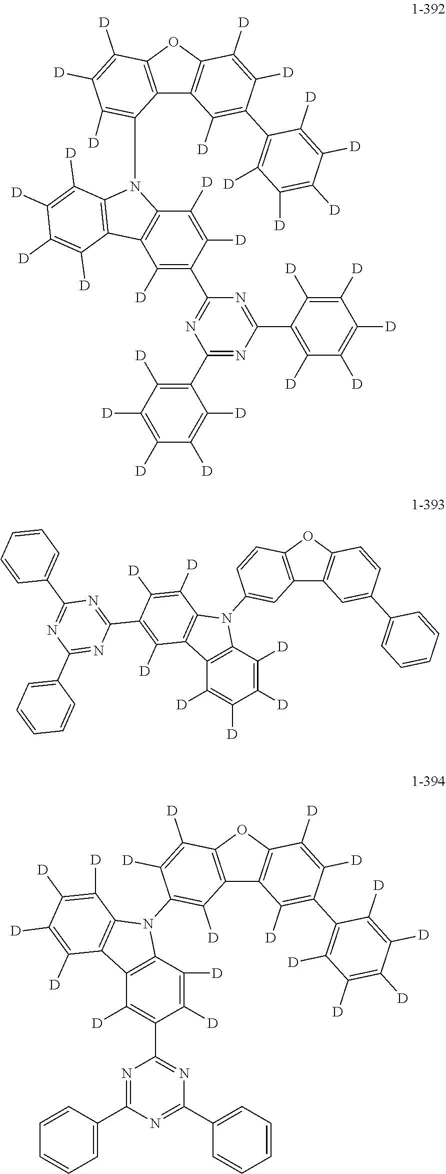

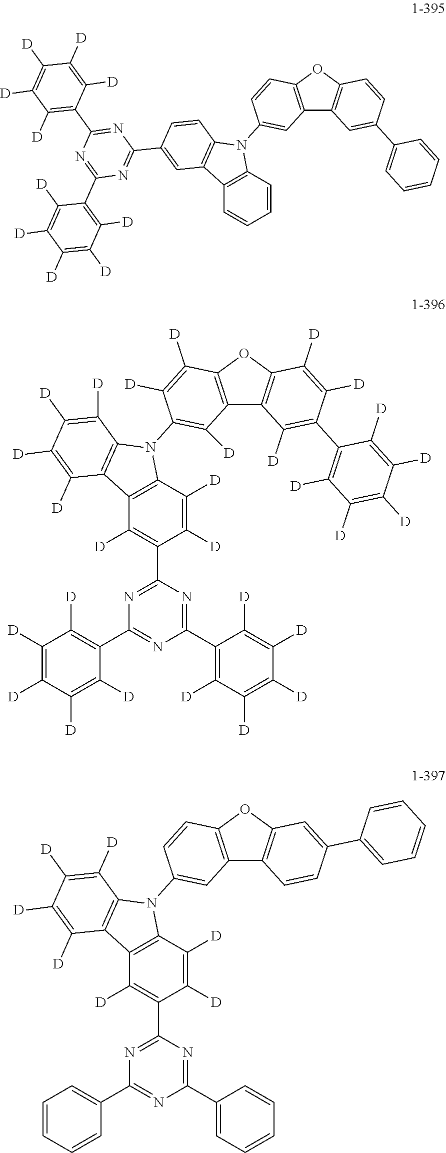

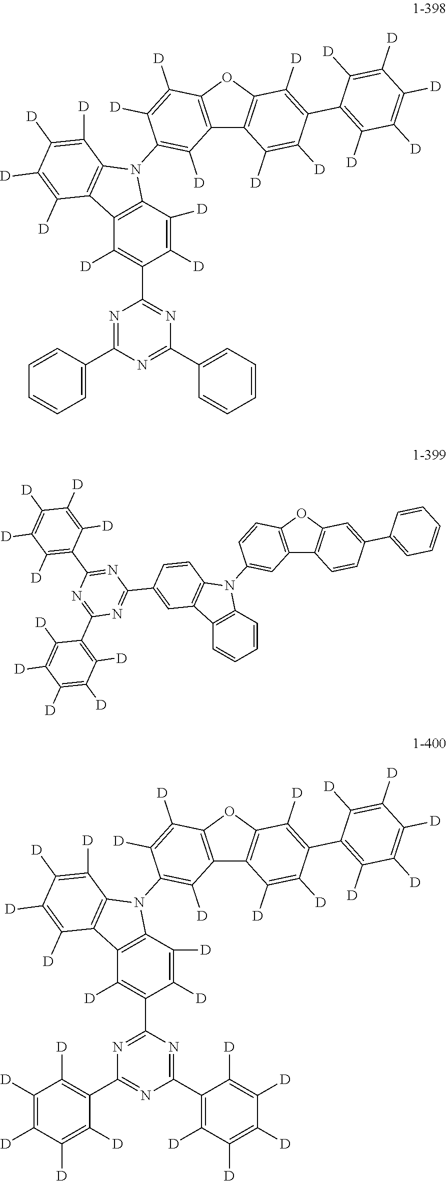

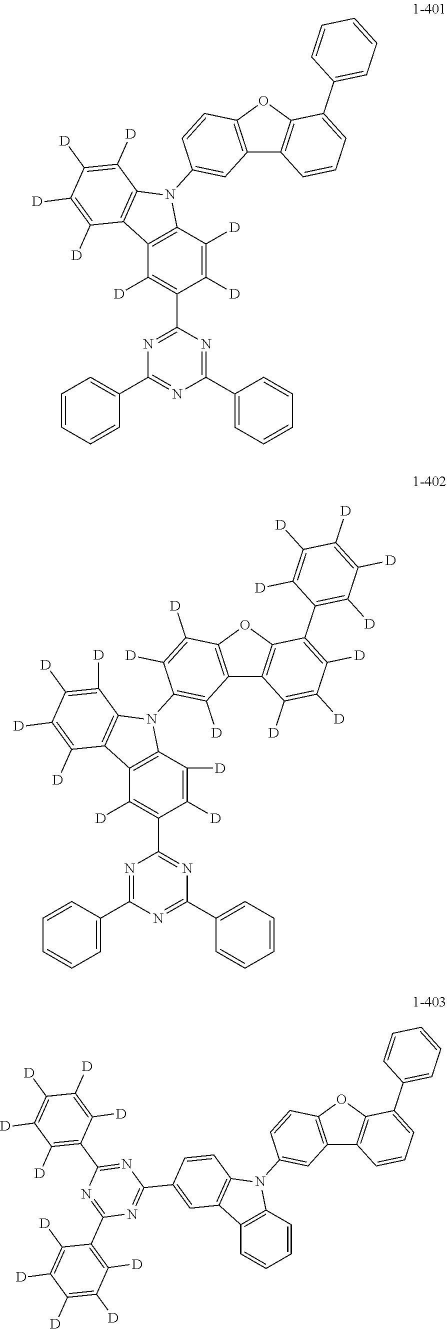 [SMILES: [2H]c1c([2H])c([2H])c2c(c1[2H])c1c([2H])c(-c3nc(-c4ccccc4)nc(-c4ccccc4)n3)c([2H])c([2H])c1n2-c1ccc2oc3c(-c4ccccc4)cccc3c2c1 ; [2H]c1c([2H])c([2H])c(-c2c([2H])c([2H])c([2H])c3c2oc2c([2H])c([2H])c(-n4c5c([2H])c([2H])c([2H])c([2H])c5c5c([2H])c(-c6nc(-c7ccccc7)nc(-c7ccccc7)n6)c([2H])c([2H])c54)c([2H])c23)c([2H])c1[2H] ; [2H]c1c([2H])c([2H])c(-c2nc(-c3ccc4c(c3)c3ccccc3n4-c3ccc4oc5c(-c6ccccc6)cccc5c4c3)nc(-c3c([2H])c([2H])c([2H])c([2H])c3[2H])n2)c([2H])c1[2H]]

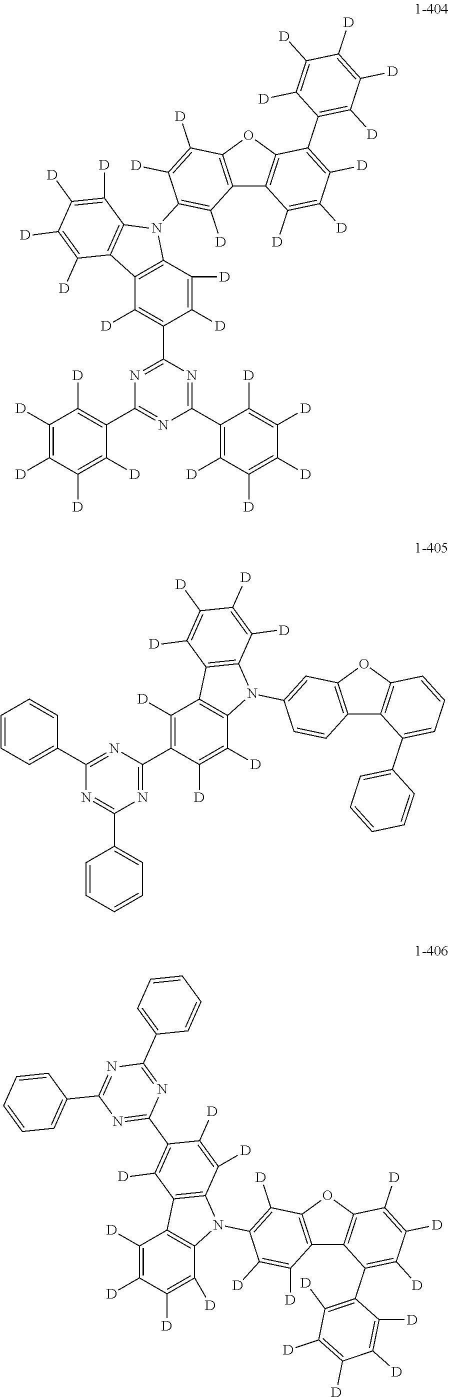

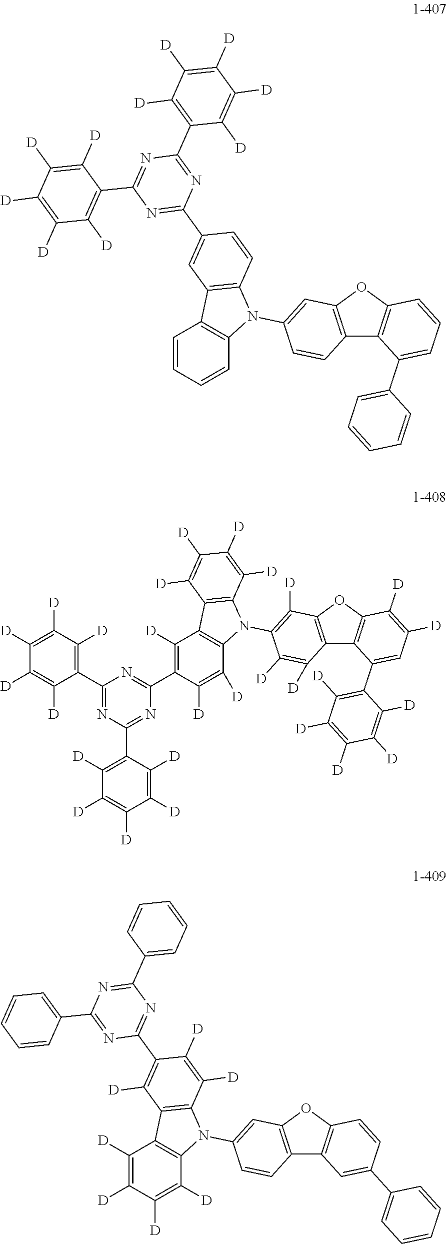

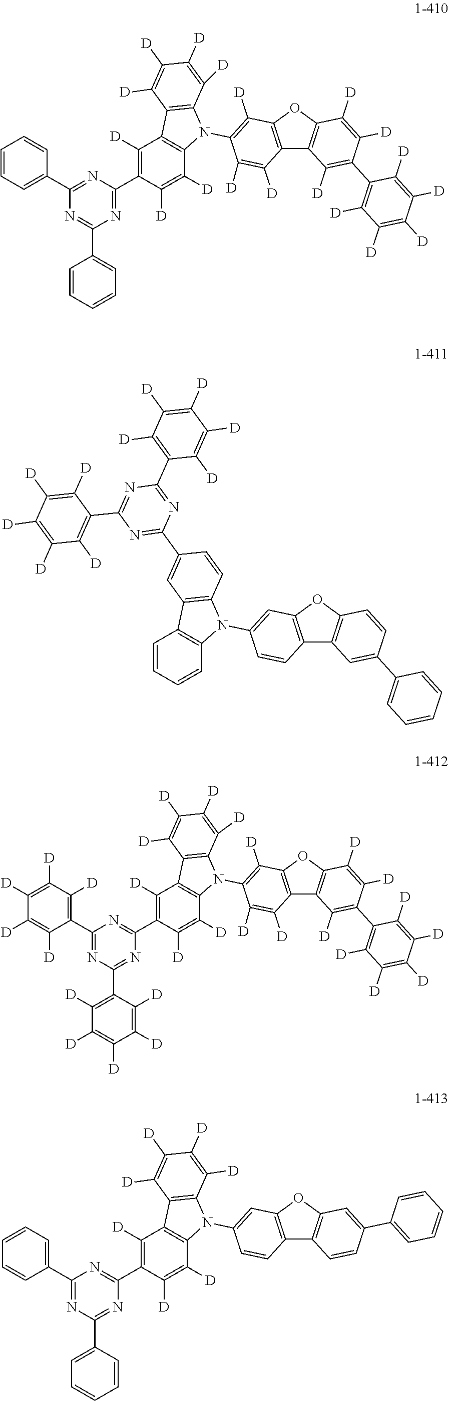

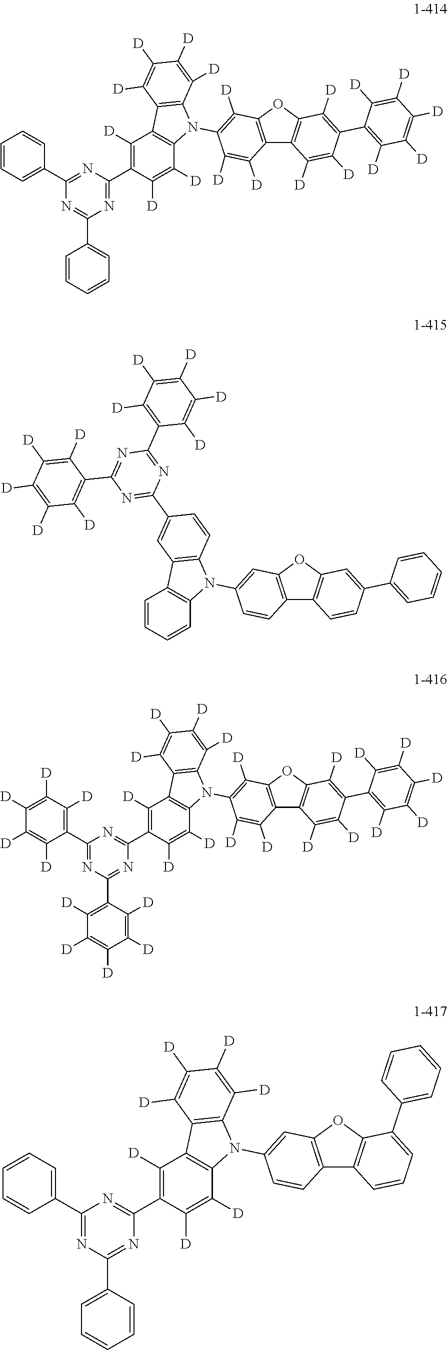

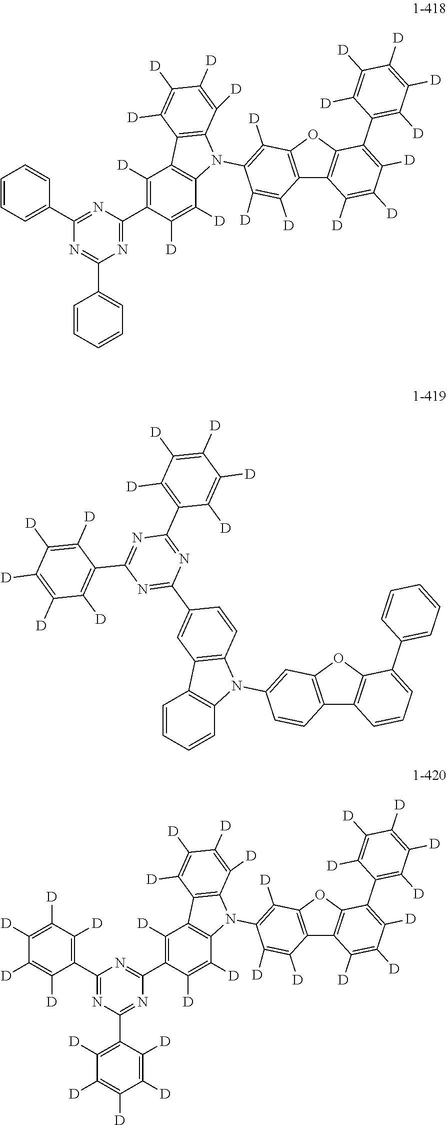

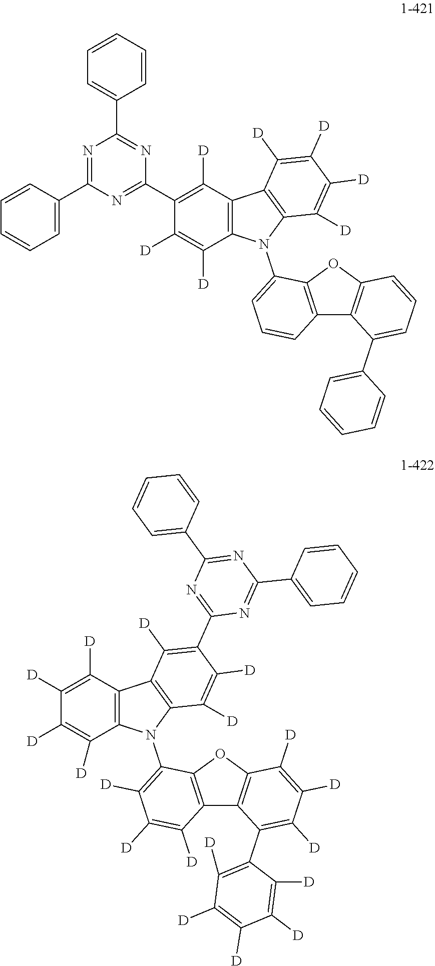 [SMILES: [2H]c1c([2H])c([2H])c2c(c1[2H])c1c([2H])c(-c3nc(-c4ccccc4)nc(-c4ccccc4)n3)c([2H])c([2H])c1n2-c1cccc2c1oc1cccc(-c3ccccc3)c12 ; [2H]c1c([2H])c([2H])c(-c2c([2H])c([2H])c([2H])c3oc4c(-n5c6c([2H])c([2H])c([2H])c([2H])c6c6c([2H])c(-c7nc(-c8ccccc8)nc(-c8ccccc8)n7)c([2H])c([2H])c65)c([2H])c([2H])c([2H])c4c23)c([2H])c1[2H]]

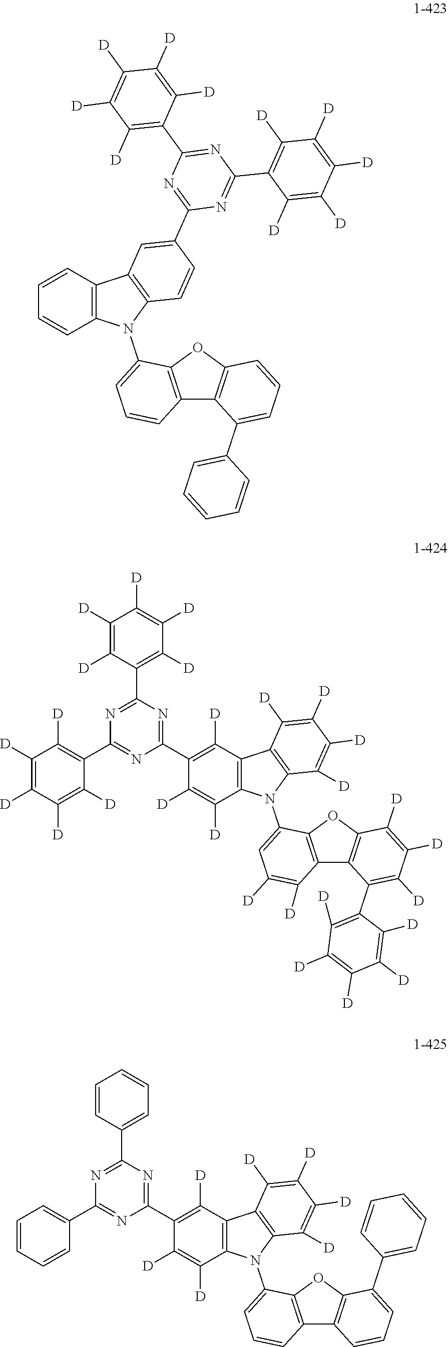

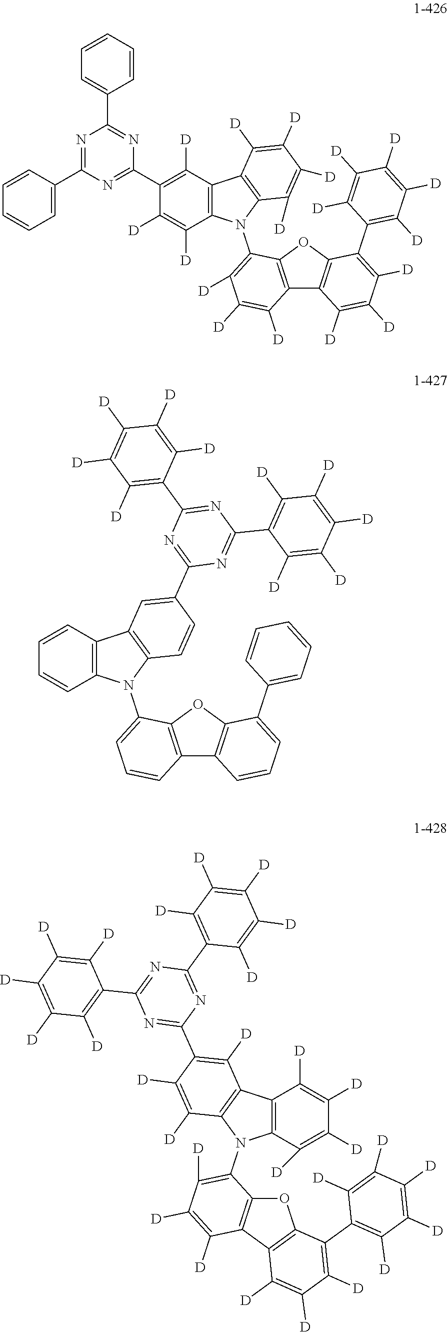

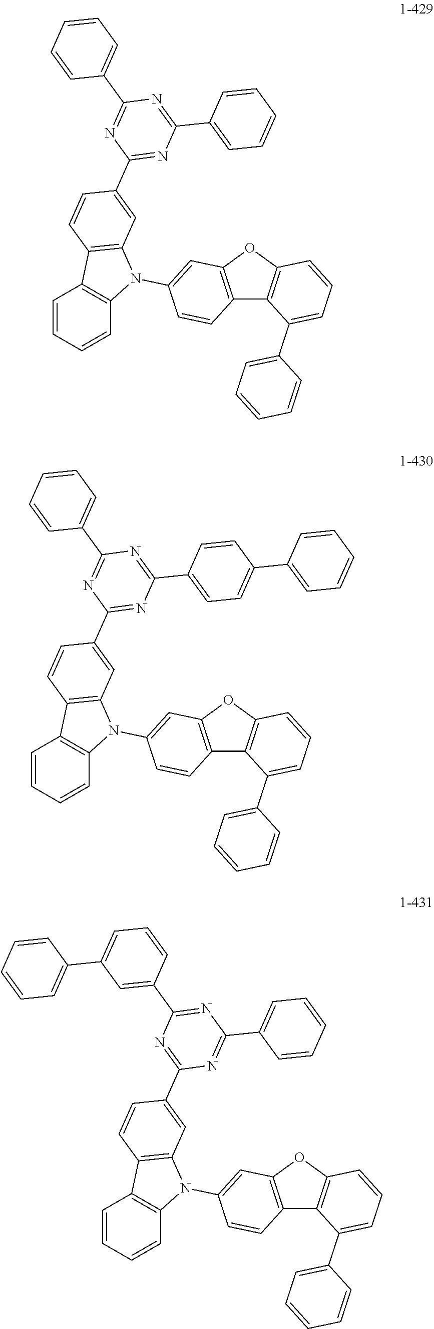 [SMILES: c1ccc(-c2nc(-c3ccccc3)nc(-c3ccc4c5ccccc5n(-c5ccc6c(c5)oc5cccc(-c7ccccc7)c56)c4c3)n2)cc1 ; c1ccc(-c2ccc(-c3nc(-c4ccccc4)nc(-c4ccc5c6ccccc6n(-c6ccc7c(c6)oc6cccc(-c8ccccc8)c67)c5c4)n3)cc2)cc1 ; c1ccc(-c2cccc(-c3nc(-c4ccccc4)nc(-c4ccc5c6ccccc6n(-c6ccc7c(c6)oc6cccc(-c8ccccc8)c67)c5c4)n3)c2)cc1]

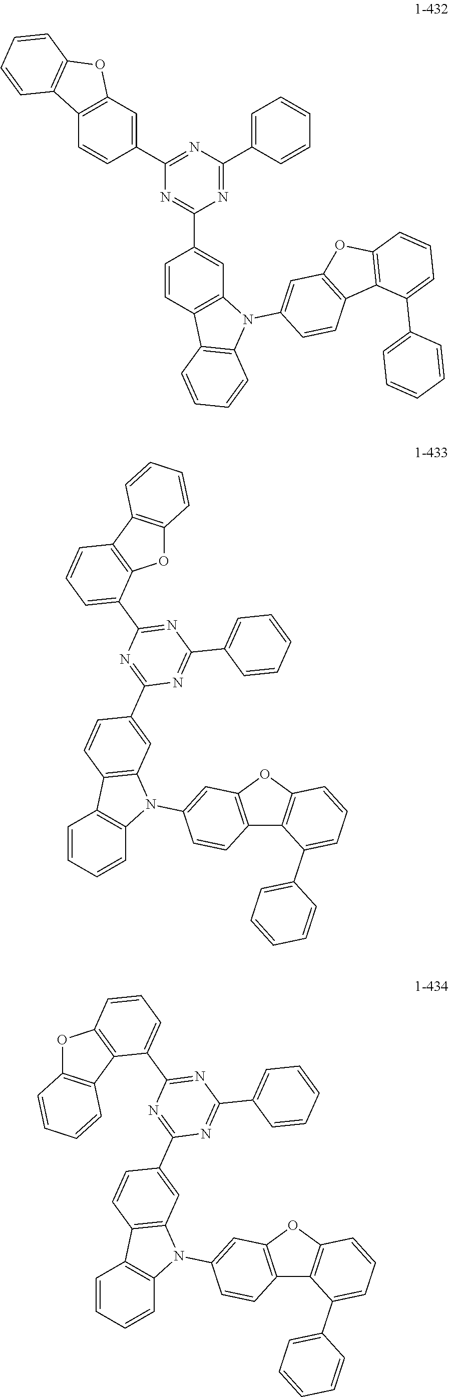 [SMILES: c1ccc(-c2nc(-c3ccc4c(c3)oc3ccccc34)nc(-c3ccc4c5ccccc5n(-c5ccc6c(c5)oc5cccc(-c7ccccc7)c56)c4c3)n2)cc1 ; c1ccc(-c2nc(-c3ccc4c5ccccc5n(-c5ccc6c(c5)oc5cccc(-c7ccccc7)c56)c4c3)nc(-c3cccc4c3oc3ccccc34)n2)cc1 ; c1ccc(-c2nc(-c3ccc4c5ccccc5n(-c5ccc6c(c5)oc5cccc(-c7ccccc7)c56)c4c3)nc(-c3cccc4oc5ccccc5c34)n2)cc1]

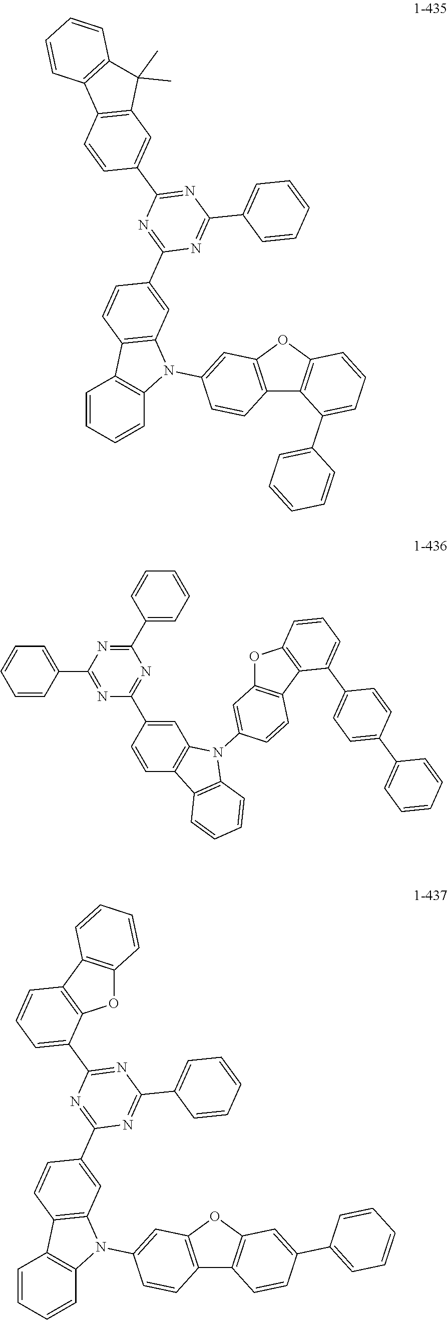 [SMILES: CC1(C)c2ccccc2-c2ccc(-c3nc(-c4ccccc4)nc(-c4ccc5c6ccccc6n(-c6ccc7c(c6)oc6cccc(-c8ccccc8)c67)c5c4)n3)cc21 ; c1ccc(-c2ccc(-c3cccc4oc5cc(-n6c7ccccc7c7ccc(-c8nc(-c9ccccc9)nc(-c9ccccc9)n8)cc76)ccc5c34)cc2)cc1 ; c1ccc(-c2ccc3c(c2)oc2cc(-n4c5ccccc5c5ccc(-c6nc(-c7ccccc7)nc(-c7cccc8c7oc7ccccc78)n6)cc54)ccc23)cc1]

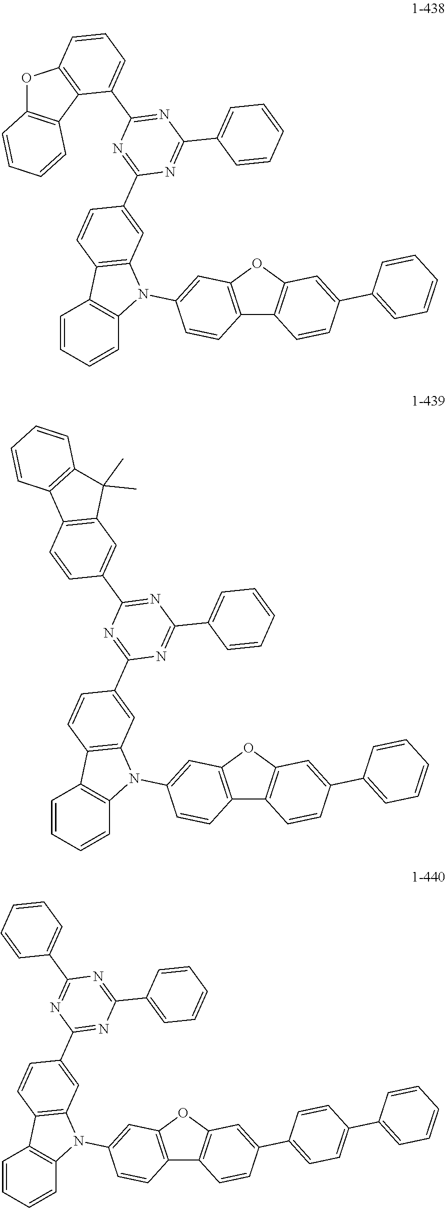 [SMILES: c1ccc(-c2ccc3c(c2)oc2cc(-n4c5ccccc5c5ccc(-c6nc(-c7ccccc7)nc(-c7cccc8oc9ccccc9c78)n6)cc54)ccc23)cc1 ; CC1(C)c2ccccc2-c2ccc(-c3nc(-c4ccccc4)nc(-c4ccc5c6ccccc6n(-c6ccc7c(c6)oc6cc(-c8ccccc8)ccc67)c5c4)n3)cc21 ; c1ccc(-c2ccc(-c3ccc4c(c3)oc3cc(-n5c6ccccc6c6ccc(-c7nc(-c8ccccc8)nc(-c8ccccc8)n7)cc65)ccc34)cc2)cc1]

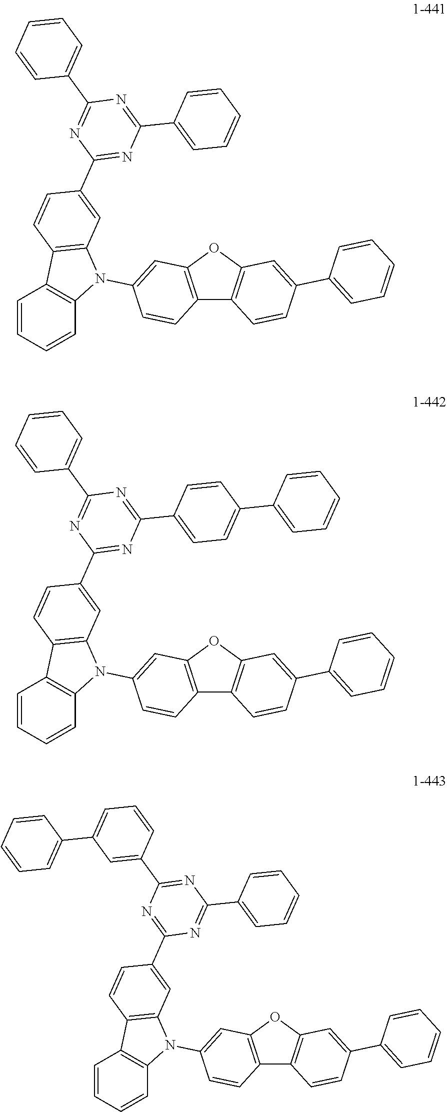 [SMILES: c1ccc(-c2ccc3c(c2)oc2cc(-n4c5ccccc5c5ccc(-c6nc(-c7ccccc7)nc(-c7ccccc7)n6)cc54)ccc23)cc1 ; c1ccc(-c2ccc(-c3nc(-c4ccccc4)nc(-c4ccc5c6ccccc6n(-c6ccc7c(c6)oc6cc(-c8ccccc8)ccc67)c5c4)n3)cc2)cc1 ; c1ccc(-c2cccc(-c3nc(-c4ccccc4)nc(-c4ccc5c6ccccc6n(-c6ccc7c(c6)oc6cc(-c8ccccc8)ccc67)c5c4)n3)c2)cc1]

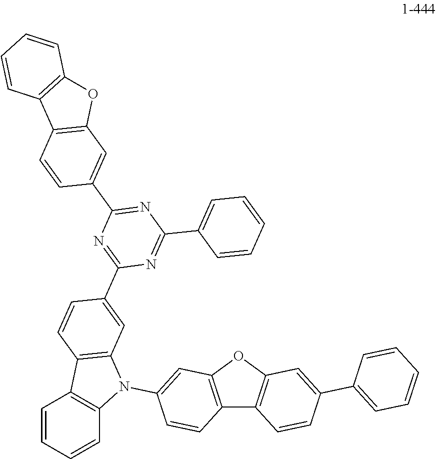 [SMILES: c1ccc(-c2ccc3c(c2)oc2cc(-n4c5ccccc5c5ccc(-c6nc(-c7ccccc7)nc(-c7ccc8c(c7)oc7ccccc78)n6)cc54)ccc23)cc1]

In addition, by introducing various substituents to the structure of Chemical Formula 1, compounds having unique properties of the introduced substituents may be synthesized. For example, by introducing substituents normally used for a hole injection layer material, a hole transport layer material, a light emitting layer material, an electron transport layer material and a charge generation layer material used for manufacturing an organic light emitting device to the core structure, materials satisfying conditions required for each organic material layer may be synthesized.

In addition, by introducing various substituents to the structure of Chemical Formula 1, the energy bandgap may be finely controlled, and meanwhile, properties at interfaces between organic materials may be enhanced, and material applications may become diverse.

Meanwhile, the heterocyclic compound has a high glass transition temperature (Tg), and thereby has excellent thermal stability. Such an increase in the thermal stability becomes an important factor providing driving stability to a device.

The heterocyclic compound according to one embodiment of the present disclosure may be prepared using a multi-step chemical reaction. Some intermediate compounds are prepared first, and from the intermediate compounds, the compound of Chemical Formula 1 may be prepared. More specifically, the heterocyclic compound according to one embodiment of the present disclosure may be prepared based on preparation examples to describe later.

In addition, one embodiment of the present disclosure relates to an organic light emitting device comprising:
a first electrode;
a second electrode provided opposite to the first electrode; and
one or more organic material layers provided between the first electrode and the second electrode,
wherein at least one of the one or more organic material layers comprise the heterocyclic compound represented by Chemical Formula 1.

The “organic light emitting device” may be expressed in terms such as an “organic light emitting diode”, an “OLED”, an “OLED device” and an “organic electroluminescent device”.

In one embodiment of the present disclosure, the first electrode may be a positive electrode, and the second electrode may be a negative electrode.

In another embodiment, the first electrode may be a negative electrode, and the second electrode may be a positive electrode.

In one embodiment of the present disclosure, the organic light emitting device may be a red organic light emitting device, and the heterocyclic compound represented by Chemical Formula 1 may be used as a material of the red organic light emitting device.

In another embodiment of the present disclosure, the organic light emitting device may be a blue organic light emitting device, and the heterocyclic compound represented by Chemical Formula 1 may be used as a material of the blue organic light emitting device.

In another embodiment of the present disclosure, the organic light emitting device may be a green organic light emitting device, and the heterocyclic compound represented by Chemical Formula 1 may be used as a material of the green organic light emitting device.

In one embodiment of the present disclosure, the organic light emitting device may be a red organic light emitting device, and the heterocyclic compound represented by Chemical Formula 1 may be used as a light emitting layer material of the red organic light emitting device.

In another embodiment of the present disclosure, the organic light emitting device may be a blue organic light emitting device, and the heterocyclic compound represented by Chemical Formula 1 may be used as a light emitting layer material of the blue organic light emitting device.

In another embodiment of the present disclosure, the organic light emitting device may be a green organic light emitting device, and the heterocyclic compound represented by Chemical Formula 1 may be used as a light emitting layer material of the green organic light emitting device.

Specific descriptions on the heterocyclic compound represented by Chemical Formula 1 are the same as the descriptions provided above.

The organic light emitting device of the present disclosure may be manufactured using common organic light emitting device manufacturing methods and materials except that one or more organic material layers are formed using the heterocyclic compound described above.

The heterocyclic compound may be formed into an organic material layer using a solution coating method as well as a vacuum deposition method when the organic light emitting device is manufactured. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may also be formed in a multilayer structure in which two or more organic material layers are laminated. For example, the organic light emitting device of the present disclosure may have a structure including a hole injection layer, an electron blocking layer, a hole transport layer, a light emitting layer, an electron transport layer, a hole blocking layer, an electron injection layer and the like as the organic material layers. However, the structure of the organic light emitting device is not limited thereto, and may include a smaller number of organic material layers.

In the organic light emitting device of the present disclosure, the organic material layer includes a light emitting layer, and the light emitting layer may include the heterocyclic compound represented by Chemical Formula 1. When the heterocyclic compound is used in the light emitting layer, driving efficiency and lifetime of the organic light emitting device may become superior since strong charge transfer is possible by spatially separating HOMO (highest occupied molecular orbital) and LUMO (lowest unoccupied molecular orbital).

One embodiment of the present disclosure provides the organic light emitting device in which the organic material layer including the heterocyclic compound represented by Chemical Formula 1 further includes a heterocyclic compound represented by the following Chemical Formula 2.

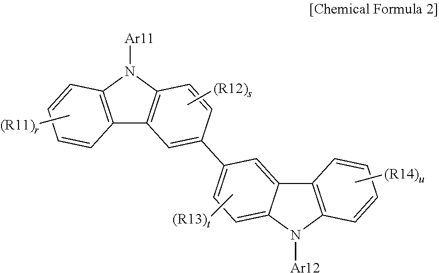

In Chemical Formula 2,
R11 to R14 are the same as or different from each other, and each independently selected from the group consisting of hydrogen; deuterium; halogen; a cyano group; a substituted or unsubstituted C1 to C60 a substituted or alkyl group; unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; and —NR201R202, or two or more groups adjacent to each other bond to each other to form a substituted or unsubstituted C6 to C60 aromatic hydrocarbon ring or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group,
Ar11 and Ar12 are the same as or different from each other, and each independently a substituted or unsubstituted C6 to C60 aryl group; or a substituted or unsubstituted C2 to C60 heteroaryl group,
r is an integer of 0 to 4, and when r is 2 or greater, R11s are the same as or different from each other,
s is an integer of 0 to 3, and when s is 2 or greater, R12s are the same as or different from each other,
t is an integer of 0 to 3, and when t is 2 or greater, R13s are the same as or different from each other, and
u is an integer of 0 to 4, and when u is 2 or greater, R14s are the same as or different from each other.

In one embodiment of the present disclosure, R11 to R14 are the same as or different from each other, and each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R201R202; —SiR201R202R203; or —NR201R202, or two or more groups adjacent to each other bond to each other to form a substituted or unsubstituted C6 to C30 aromatic hydrocarbon ring or a substituted or unsubstituted C2 to C30 heteroring, and R201, R202 and R203 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present disclosure, R11 to R14 are the same as or different from each other, and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R201R202; —SiR201R202R203; or —NR201R202, or two or more groups adjacent to each other bond to each other to form a substituted or unsubstituted C6 to C20 aromatic hydrocarbon ring or a substituted or unsubstituted C2 to C20 heteroring, and R201, R202 and R203 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R11 to R14 are the same as or different from each other, and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; or a substituted or unsubstituted C2 to C20 heteroaryl group.

In another embodiment of the present disclosure, R11 to R14 are the same as or different from each other, and may be each independently hydrogen; deuterium; a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R11 to R14 are the same as or different from each other, and may be each independently hydrogen; or deuterium.

In one embodiment of the present disclosure, Ar1 l and Ar12 are the same as or different from each other, and may be each independently a substituted or unsubstituted C6 to C30 aryl group; or a substituted or unsubstituted C2 to C30 heteroaryl group.

In another embodiment of the present disclosure, Ar1 l and Ar12 are the same as or different from each other, and may be each independently a substituted or unsubstituted C6 to C20 aryl group; or a substituted or unsubstituted C2 to C20 heteroaryl group.

In another embodiment of the present disclosure, Ar11 and Ar12 are the same as or different from each other, and may be each independently a substituted or unsubstituted C6 to C20 aryl group.

In another embodiment of the present disclosure, Ar1 l may be a substituted or unsubstituted phenyl group; a substituted or unsubstituted biphenyl group; a substituted or unsubstituted terphenyl group; a substituted or unsubstituted naphthyl group; a substituted or unsubstituted triphenylenyl group; a substituted or unsubstituted fluorenyl group; or a substituted or unsubstituted spirobifluorenyl group.

The substituent of the phenyl group may be a cyano group or —SiR201R202R203, and R201 to R203 may all be a substituted or unsubstituted phenyl group.

In another embodiment of the present disclosure, Ar12 may be a substituted or unsubstituted phenyl group; a substituted or unsubstituted biphenyl group; a substituted or unsubstituted terphenyl group; a substituted or unsubstituted naphthyl group; a substituted or unsubstituted triphenylenyl group; or a substituted or unsubstituted fluorenyl group.

The substituent of the phenyl group may be a cyano group or —SiR201R202R203, and R201 to R203 may all be a substituted or unsubstituted phenyl group.

In one embodiment of the present disclosure, the heterocyclic compound represented by Chemical Formula 2 may not include deuterium as a substituent, or may have a deuterium content of, for example, greater than 0%, 1% or greater, 10% or greater, 20% or greater, 30% or greater, 40% or greater or 50% or greater, and 100% or less, 90% or less, 80% or less, 70% or less or 60% or less with respect to the total number of hydrogen atoms and deuterium atoms.

In another embodiment of the present disclosure, the heterocyclic compound represented by Chemical Formula 2 may not include deuterium as a substituent, or may have a deuterium content of 1% to 100% with respect to the total number of hydrogen atoms and deuterium atoms.

In another embodiment of the present disclosure, the heterocyclic compound represented by Chemical Formula 2 may not include deuterium as a substituent, or may have a deuterium content of 20% to 90% with respect to the total number of hydrogen atoms and deuterium atoms.

In another embodiment of the present disclosure, the heterocyclic compound represented by Chemical Formula 2 may not include deuterium as a substituent, or may have a deuterium content of 30% to 80% with respect to the total number of hydrogen atoms and deuterium atoms.

In another embodiment of the present disclosure, the heterocyclic compound represented by Chemical Formula 2 may not include deuterium as a substituent, or may have a deuterium content of 50% to 70% with respect to the total number of hydrogen atoms and deuterium atoms.

When the heterocyclic compound represented by Chemical Formula 1 and the heterocyclic compound represented by Chemical Formula 2 are included at the same time, effects of superior efficiency and lifetime are obtained. This may lead to a forecast that an exciplex phenomenon occurs when the two compounds are included at the same time.

The exciplex phenomenon is a phenomenon of releasing energy having sizes of a HOMO energy level of a donor (p-host) and a LUMO energy level of an acceptor (n-host) due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transport ability and an acceptor (n-host) having a favorable electron transport ability are used as a host of a light emitting layer, holes are injected to the p-host and electrons are injected to the n-host, and thus a driving voltage may be lowered, which resultantly helps with enhancement in the lifetime. In other words, when the compound represented by Chemical Formula 1 is used as the donor and the compound represented by Chemical Formula 2 is used as the acceptor, excellent device properties are obtained.

In one embodiment of the present disclosure, when the heterocyclic compound represented by Chemical Formula 1 and the heterocyclic compound represented by Chemical Formula 2 are included at the same time, at least one of the compounds may not include deuterium as a substituent, or may have a deuterium content of greater than 0%, 1% or greater, 10% or greater, 20% or greater, 30% or greater, 40% or greater or 50% or greater, and 100% or less, 90% or less, 80% or less, 70% or less or 60% or less with respect to the total number of hydrogen atoms and deuterium atoms.

In another embodiment of the present disclosure, when the heterocyclic compound represented by Chemical Formula 1 and the heterocyclic compound represented by Chemical Formula 2 are included at the same time, at least one of the compounds may not include deuterium as a substituent, or may have a deuterium content of 1% to 100% with respect to the total number of hydrogen atoms and deuterium atoms.

In another embodiment of the present disclosure, when the heterocyclic compound represented by Chemical Formula 1 and the heterocyclic compound represented by Chemical Formula 2 are included at the same time, at least one of the compounds may not include deuterium as a substituent, or may have a deuterium content of 20% to 90% with respect to the total number of hydrogen atoms and deuterium atoms.

In another embodiment of the present disclosure, when the heterocyclic compound represented by Chemical Formula 1 and the heterocyclic compound represented by Chemical Formula 2 are included at the same time, at least one of the compounds may not include deuterium as a substituent, or may have a deuterium content of 30% to 80% with respect to the total number of hydrogen atoms and deuterium atoms.

In another embodiment of the present disclosure, when the heterocyclic compound represented by Chemical Formula 1 and the heterocyclic compound represented by Chemical Formula 2 are included at the same time, at least one of the compounds may not include deuterium as a substituent, or may have a deuterium content of 50% to 70% with respect to the total number of hydrogen atoms and deuterium atoms.

In one embodiment of the present disclosure, the heterocyclic compound represented by Chemical Formula 2 may be one or more types selected from among the following compounds.

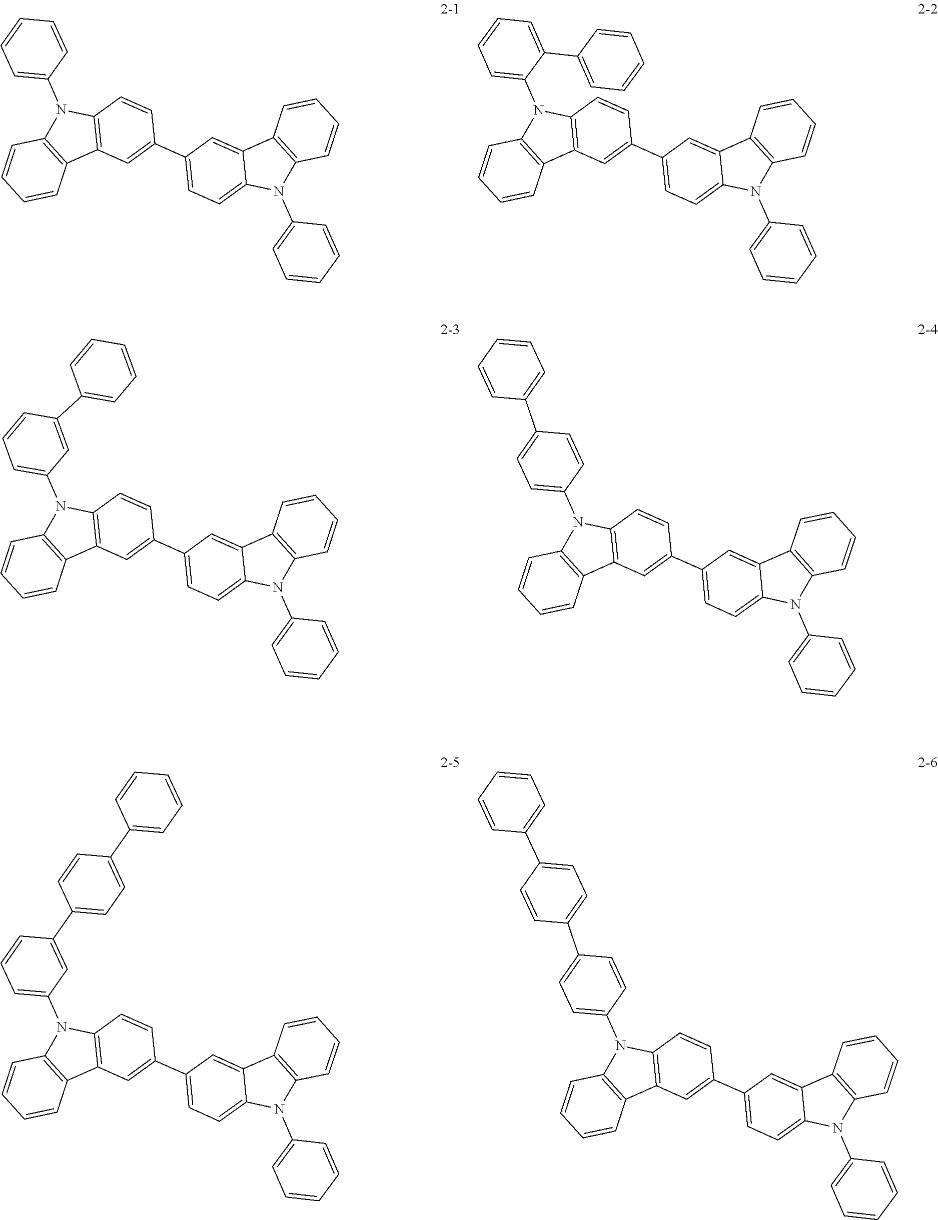 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

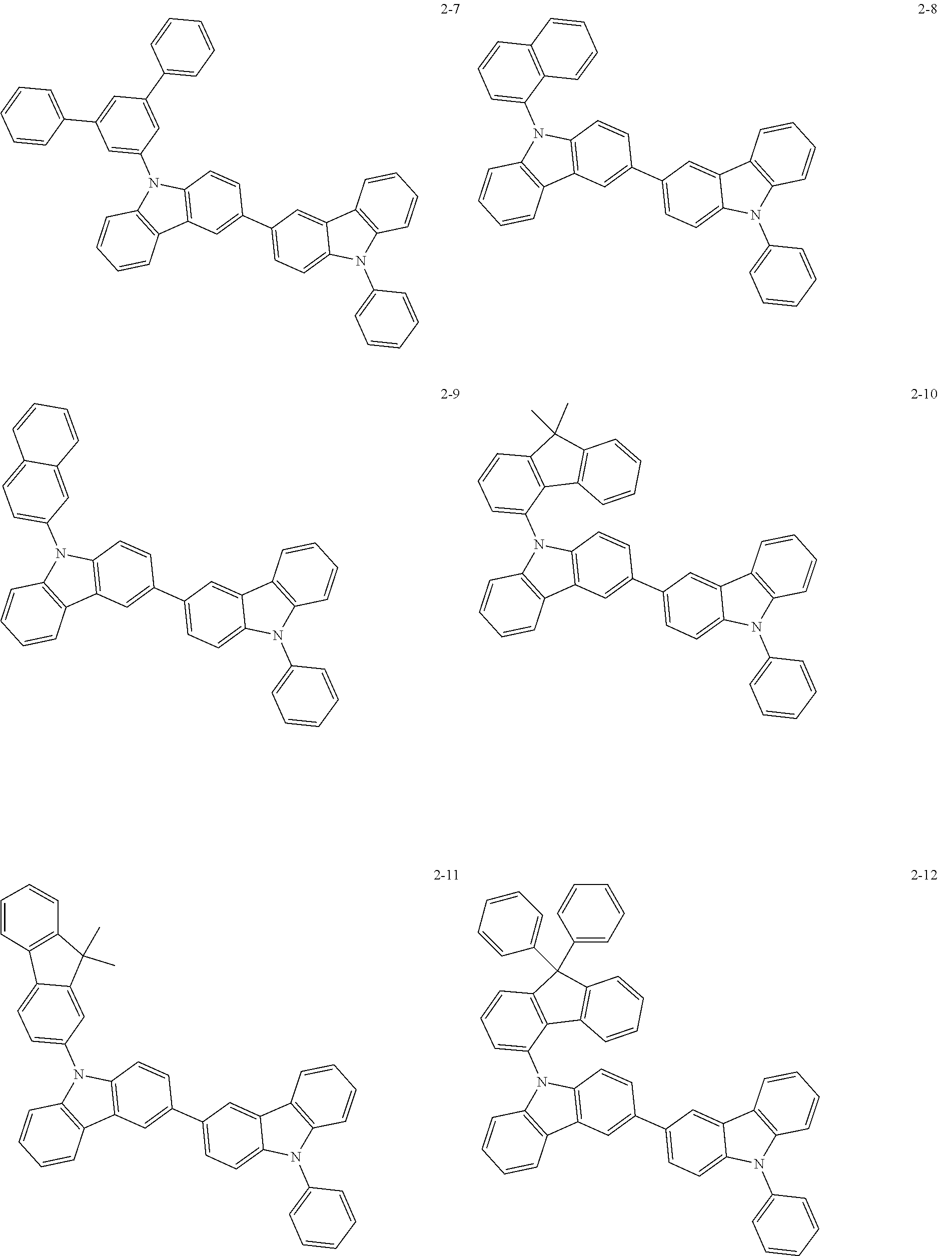 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

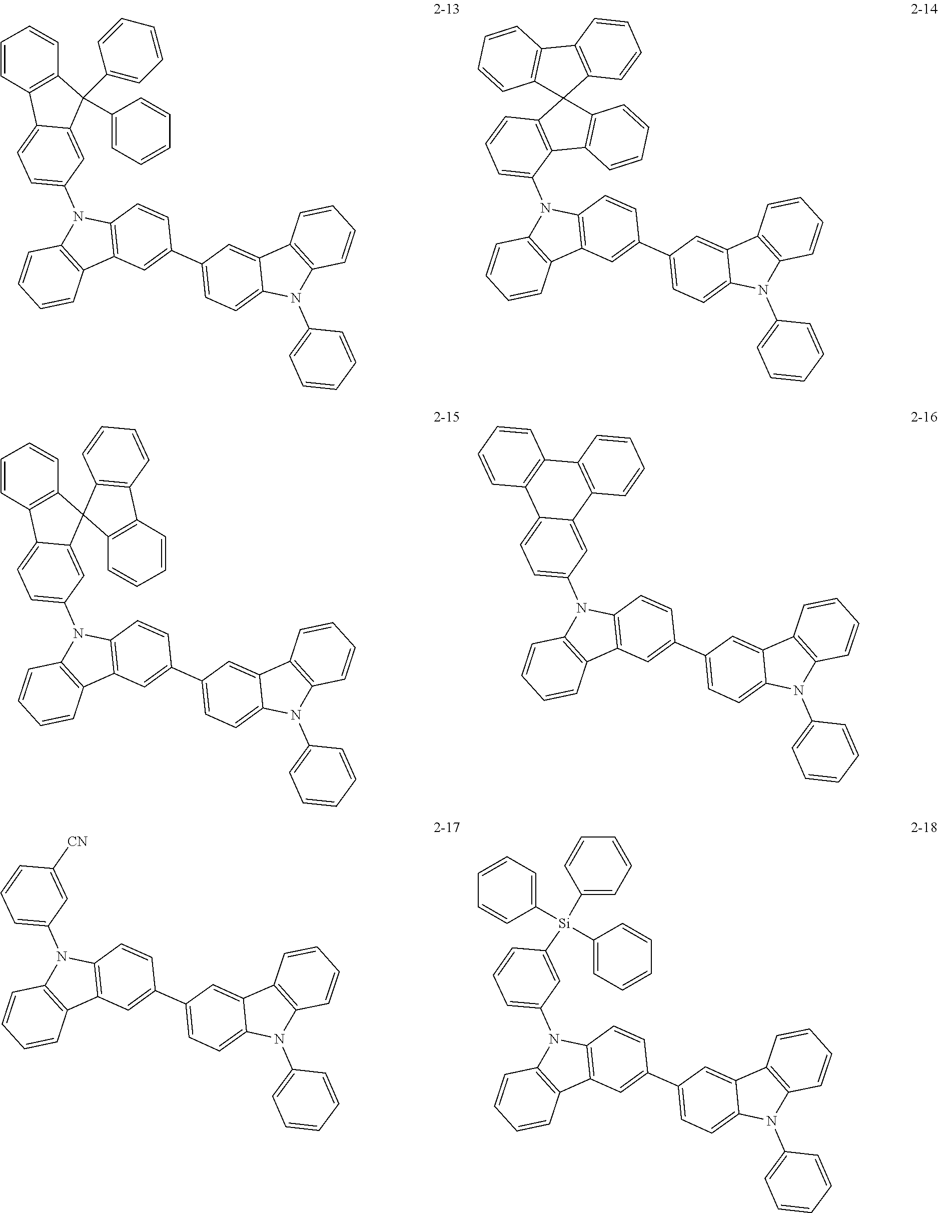

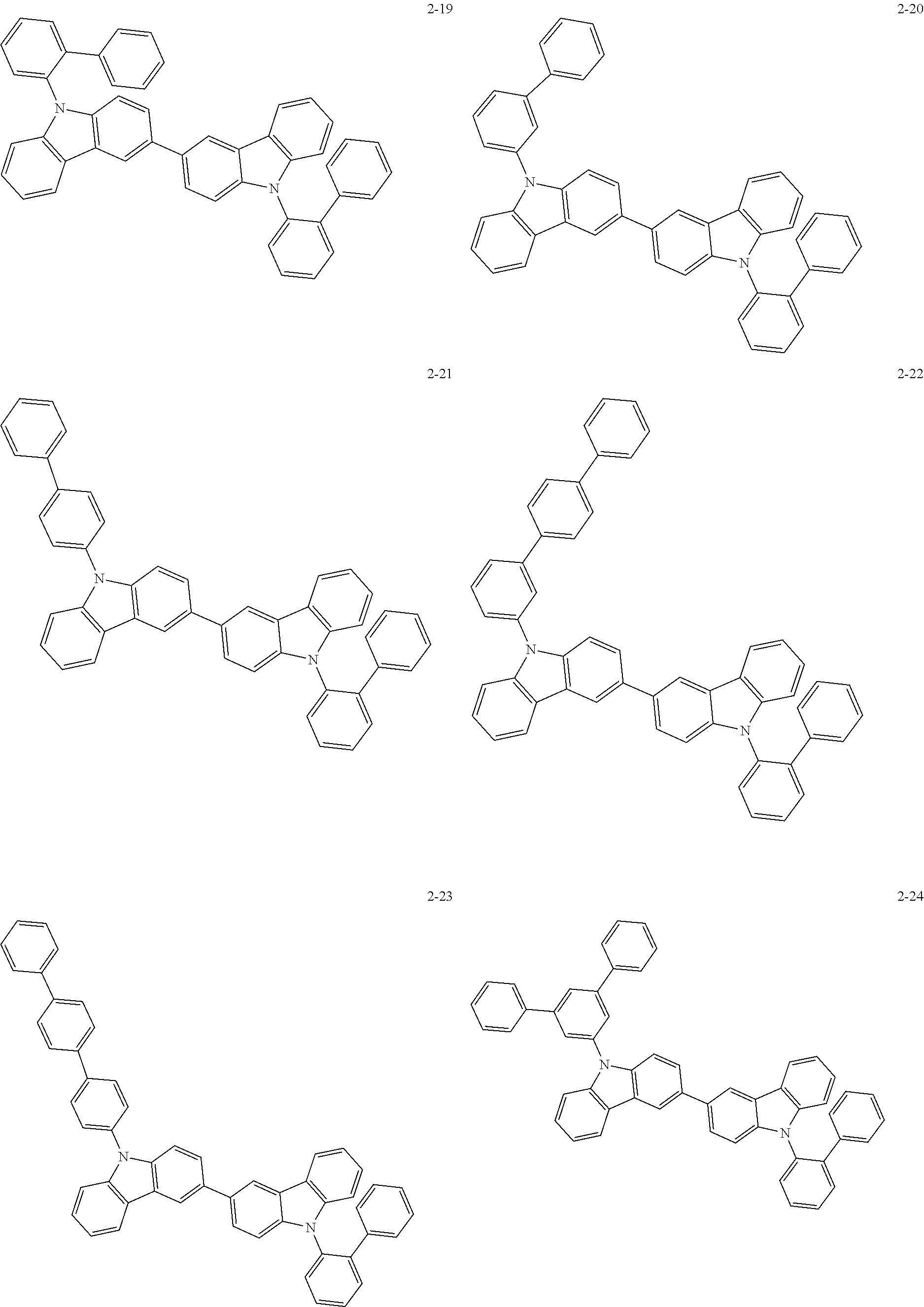

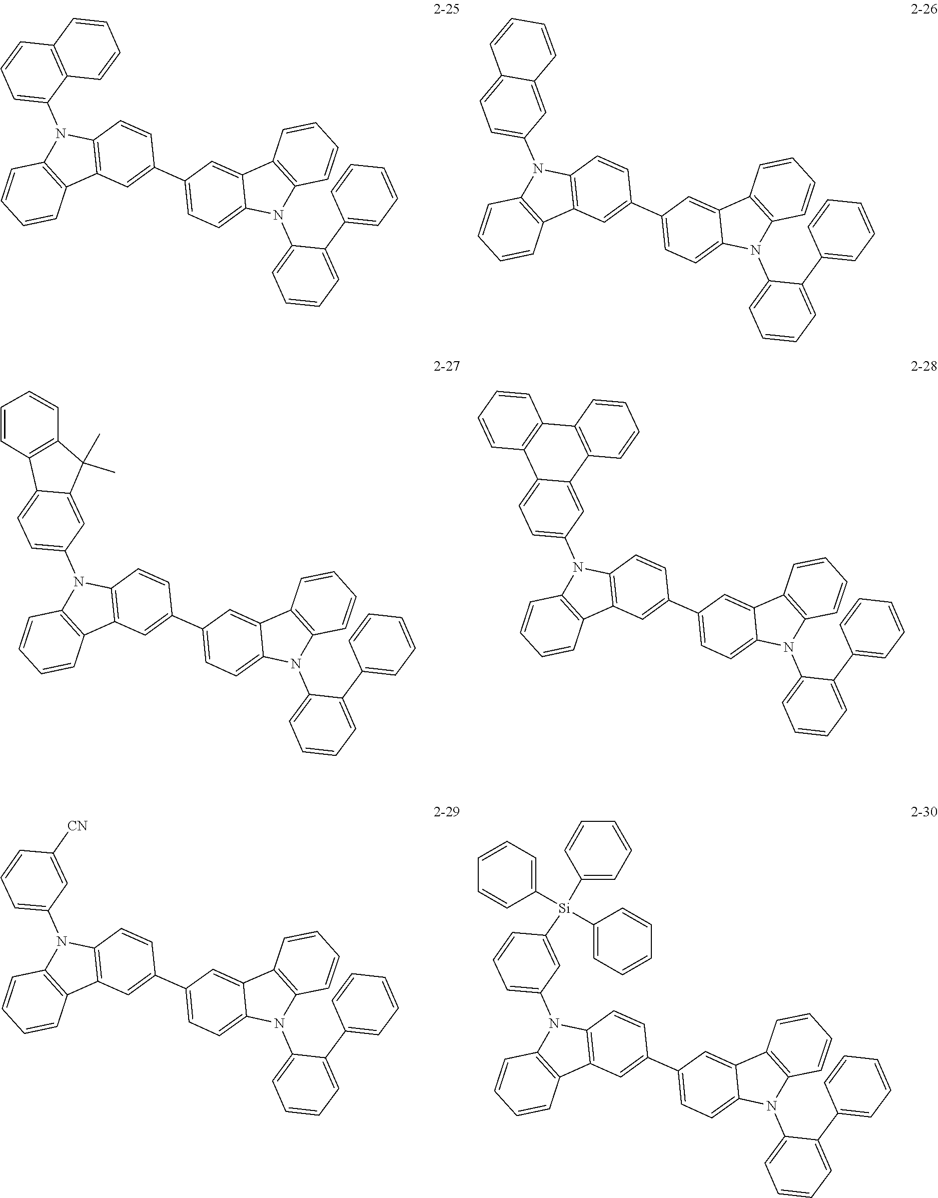

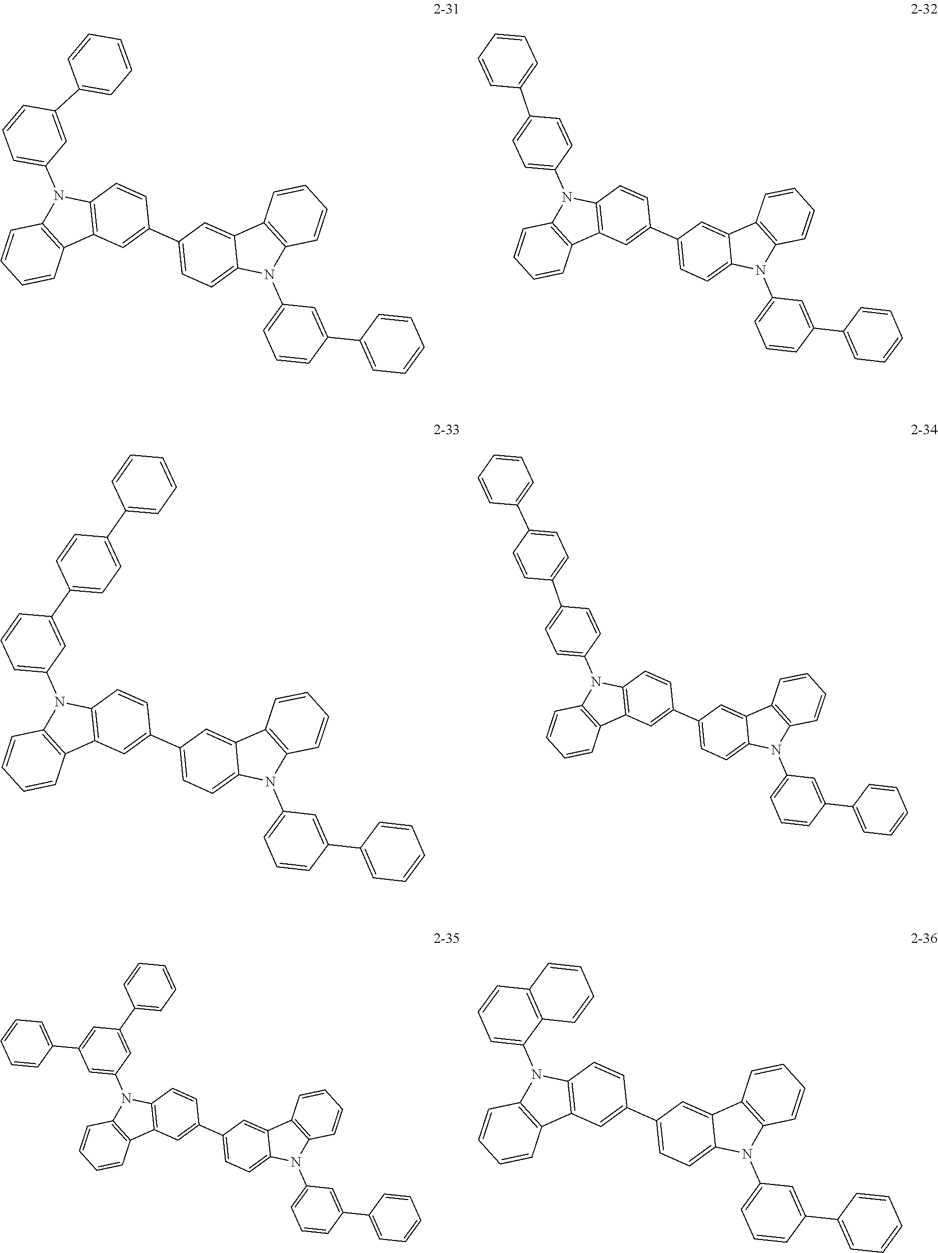

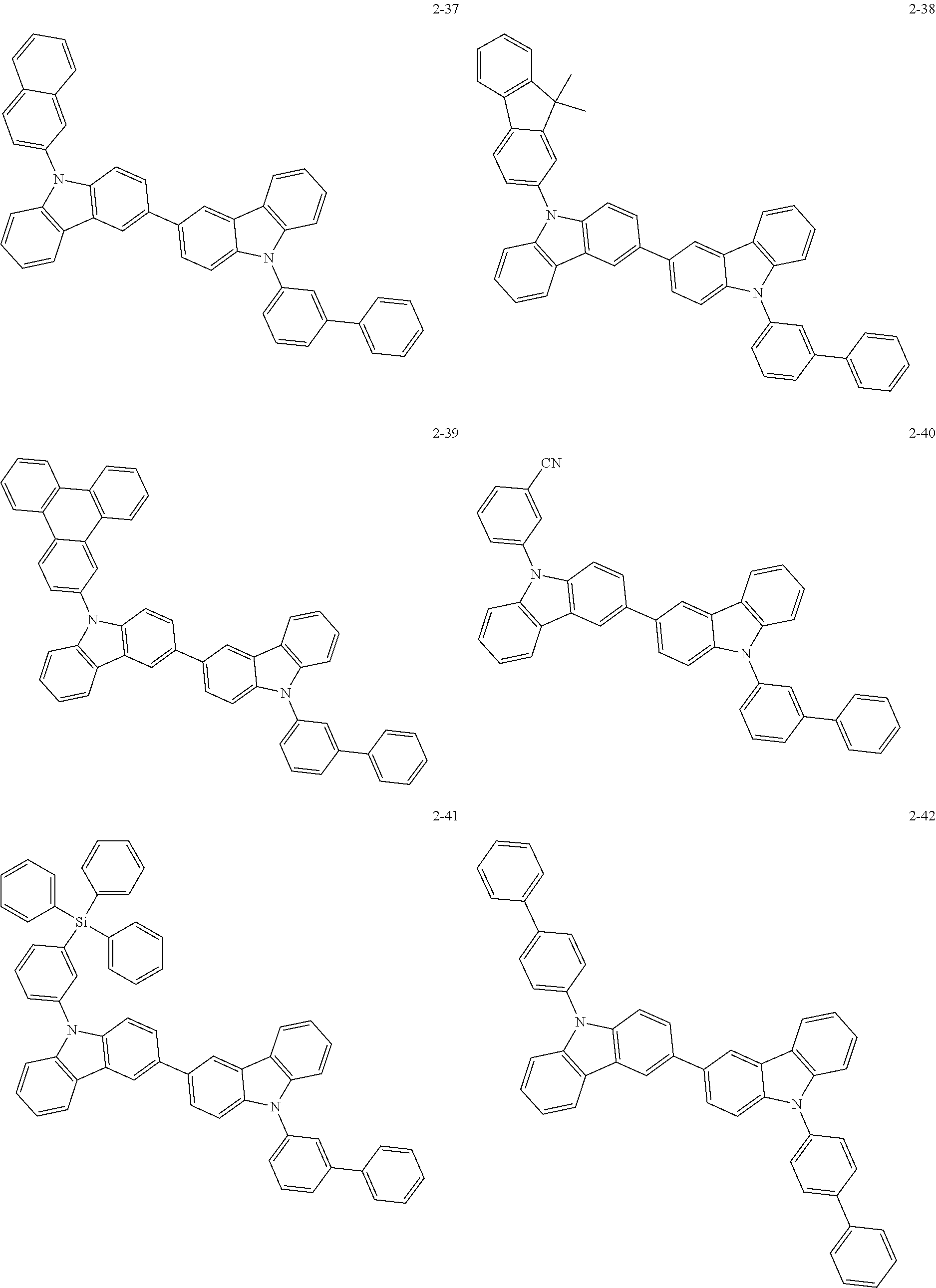

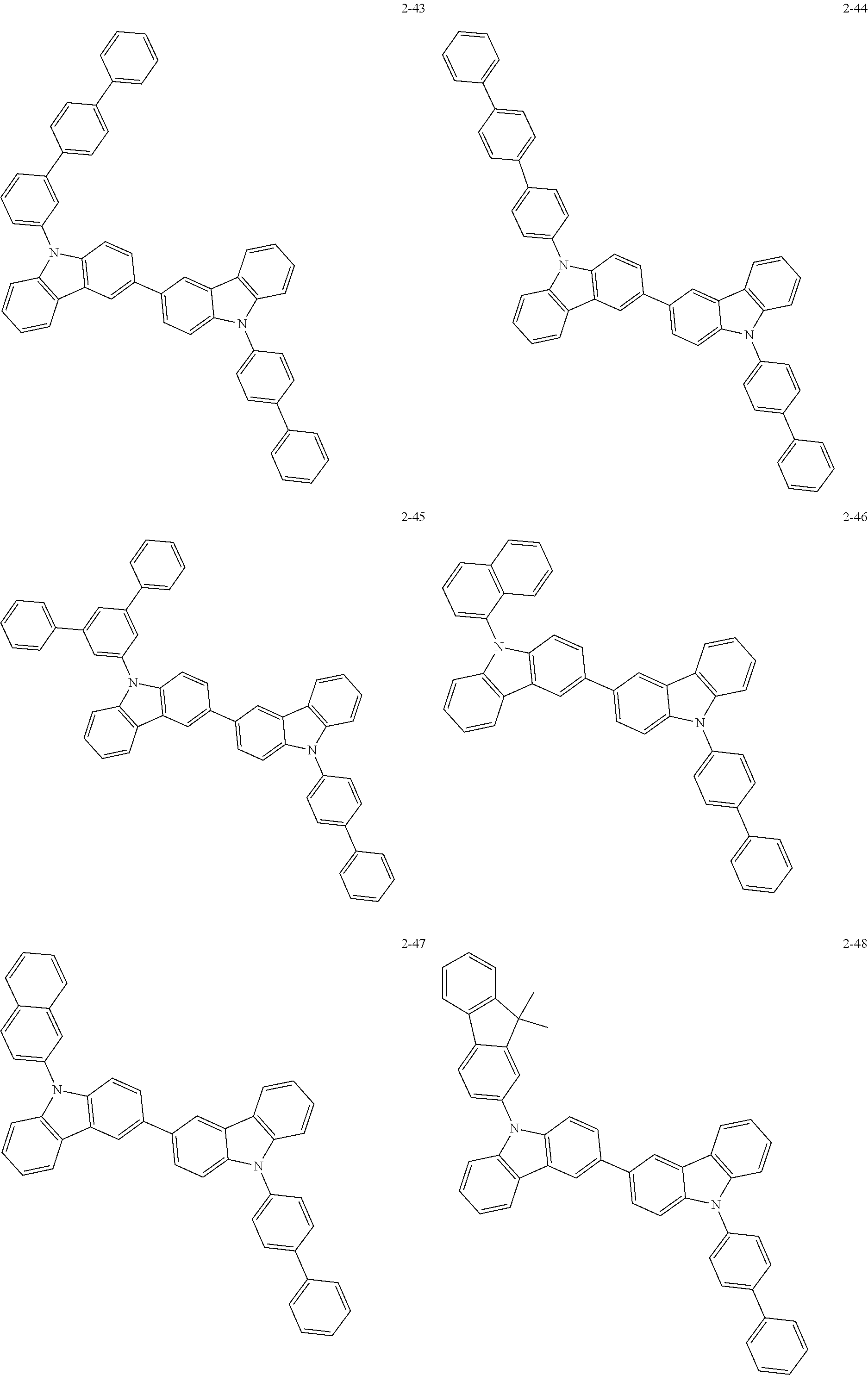

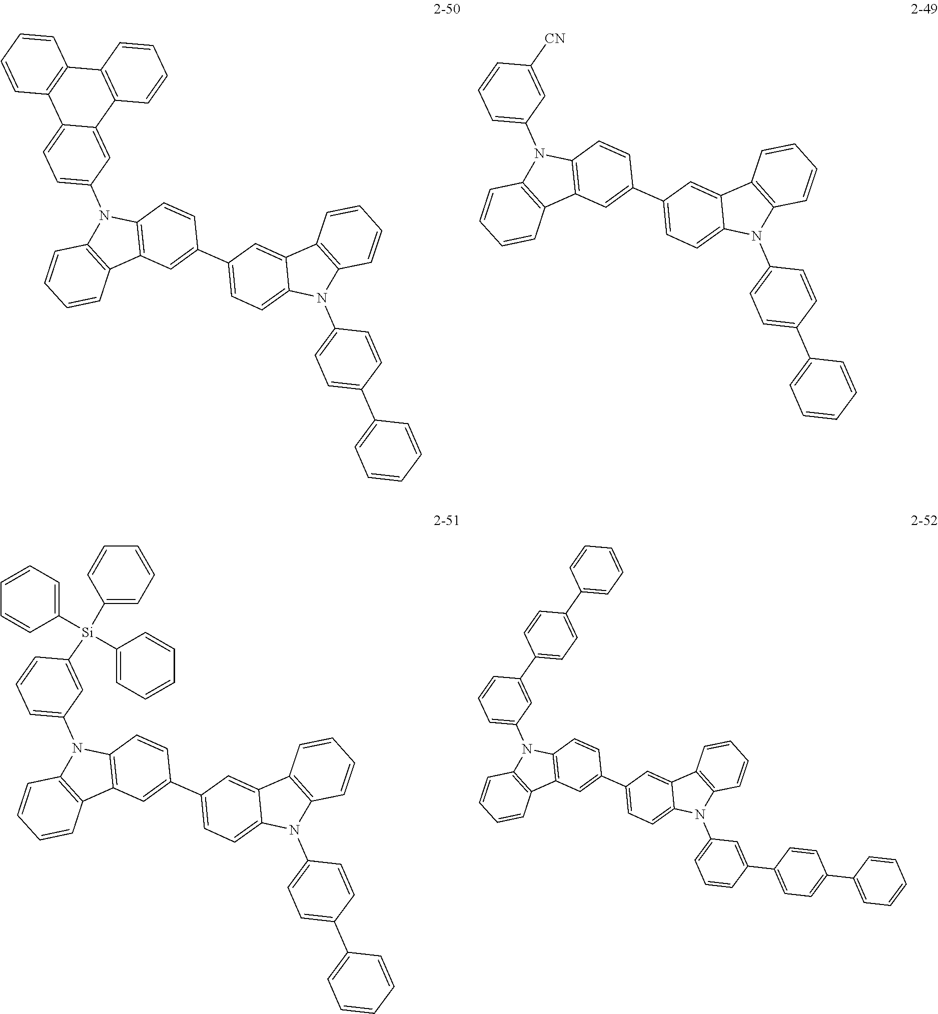 [SMILES: c1ccc(-c2ccc(-n3c4ccccc4c4cc(-c5ccc6c(c5)c5ccccc5n6-c5ccc6c7ccccc7c7ccccc7c6c5)ccc43)cc2)cc1 ; N#Cc1cccc(-n2c3ccccc3c3cc(-c4ccc5c(c4)c4ccccc4n5-c4ccc(-c5ccccc5)cc4)ccc32)c1 ; c1ccc(-c2ccc(-n3c4ccccc4c4cc(-c5ccc6c(c5)c5ccccc5n6-c5cccc([Si](c6ccccc6)(c6ccccc6)c6ccccc6)c5)ccc43)cc2)cc1 ; c1ccc(-c2ccc(-c3cccc(-n4c5ccccc5c5cc(-c6ccc7c(c6)c6ccccc6n7-c6cccc(-c7ccc(-c8ccccc8)cc7)c6)ccc54)c3)cc2)cc1]

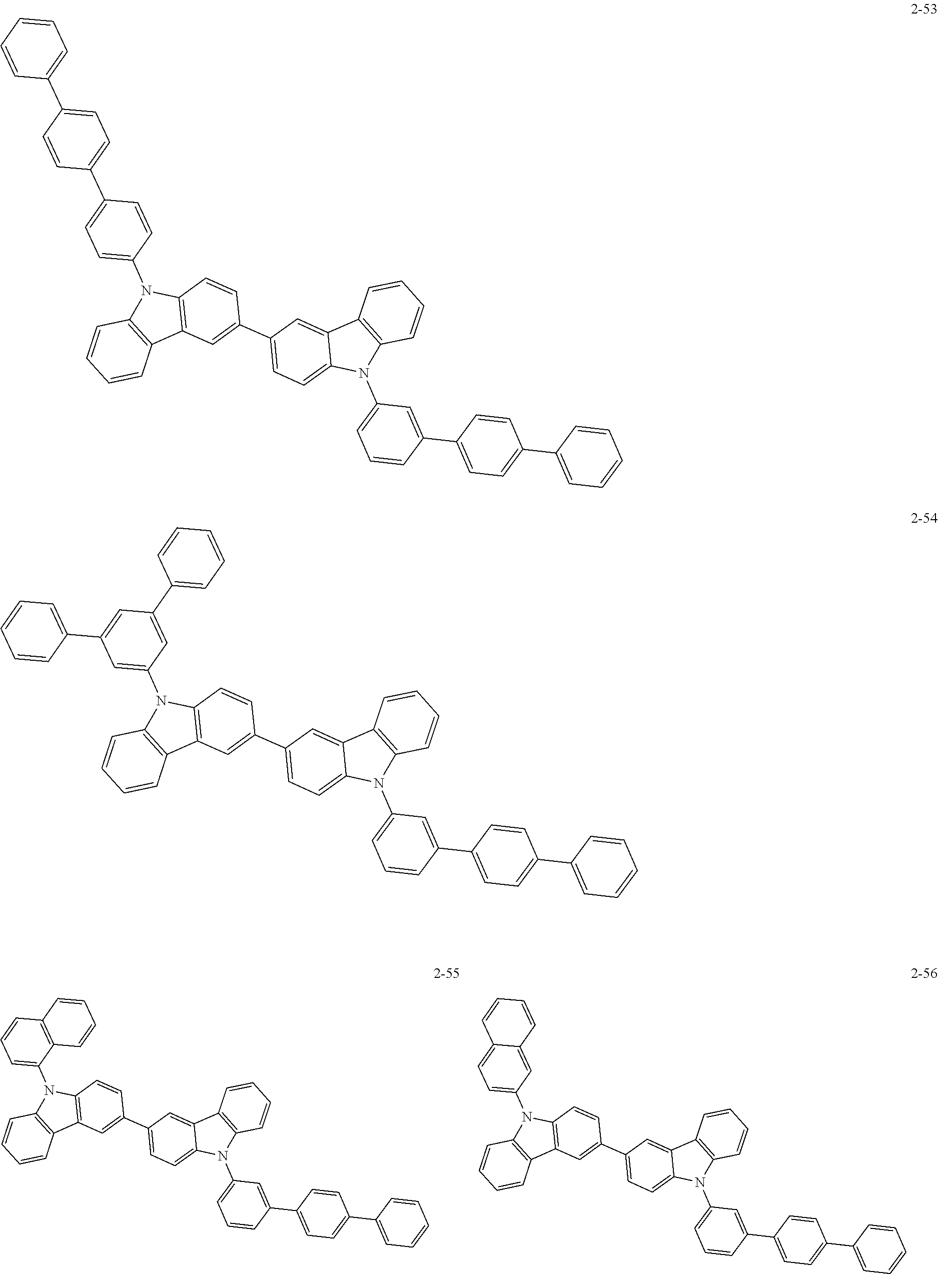 [SMILES: c1ccc(-c2ccc(-c3ccc(-n4c5ccccc5c5cc(-c6ccc7c(c6)c6ccccc6n7-c6cccc(-c7ccc(-c8ccccc8)cc7)c6)ccc54)cc3)cc2)cc1 ; c1ccc(-c2ccc(-c3cccc(-n4c5ccccc5c5cc(-c6ccc7c(c6)c6ccccc6n7-c6cc(-c7ccccc7)cc(-c7ccccc7)c6)ccc54)c3)cc2)cc1 ; c1ccc(-c2ccc(-c3cccc(-n4c5ccccc5c5cc(-c6ccc7c(c6)c6ccccc6n7-c6cccc7ccccc67)ccc54)c3)cc2)cc1 ; c1ccc(-c2ccc(-c3cccc(-n4c5ccccc5c5cc(-c6ccc7c(c6)c6ccccc6n7-c6ccc7ccccc7c6)ccc54)c3)cc2)cc1]

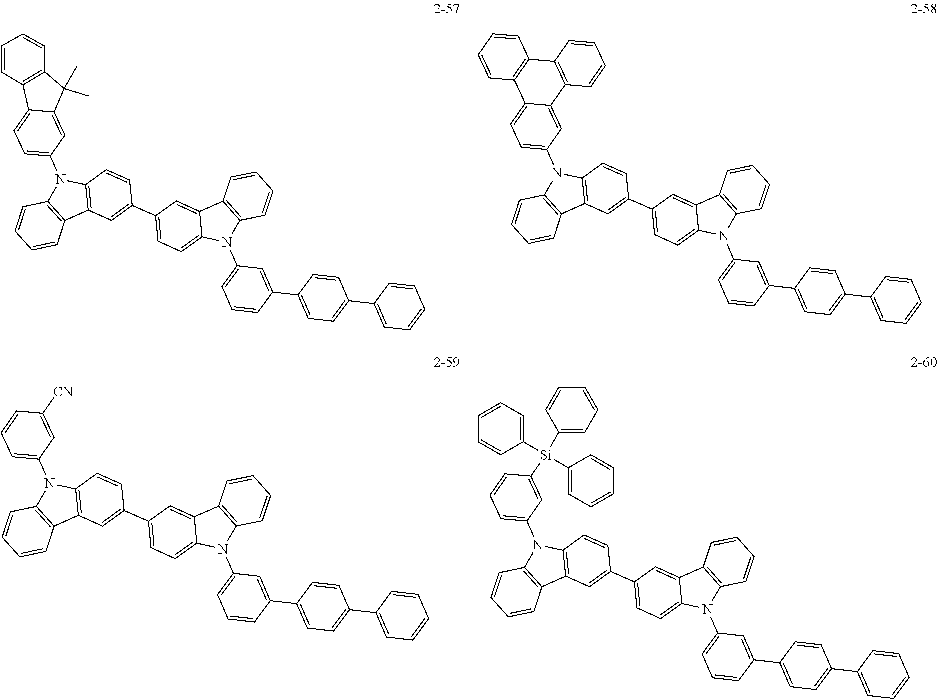 [SMILES: CC1(C)c2ccccc2-c2ccc(-n3c4ccccc4c4cc(-c5ccc6c(c5)c5ccccc5n6-c5cccc(-c6ccc(-c7ccccc7)cc6)c5)ccc43)cc21 ; c1ccc(-c2ccc(-c3cccc(-n4c5ccccc5c5cc(-c6ccc7c(c6)c6ccccc6n7-c6ccc7c8ccccc8c8ccccc8c7c6)ccc54)c3)cc2)cc1 ; N#Cc1cccc(-n2c3ccccc3c3cc(-c4ccc5c(c4)c4ccccc4n5-c4cccc(-c5ccc(-c6ccccc6)cc5)c4)ccc32)c1 ; c1ccc(-c2ccc(-c3cccc(-n4c5ccccc5c5cc(-c6ccc7c(c6)c6ccccc6n7-c6cccc([Si](c7ccccc7)(c7ccccc7)c7ccccc7)c6)ccc54)c3)cc2)cc1]

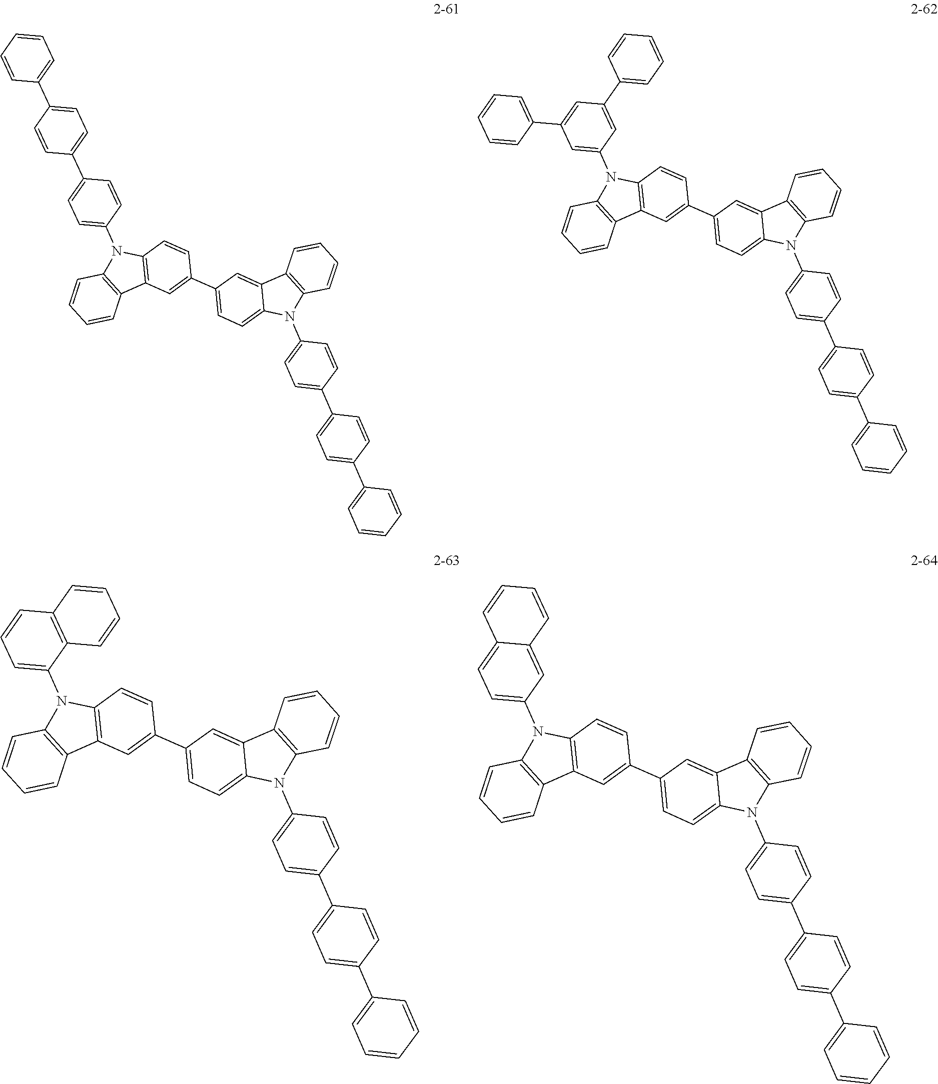 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1]

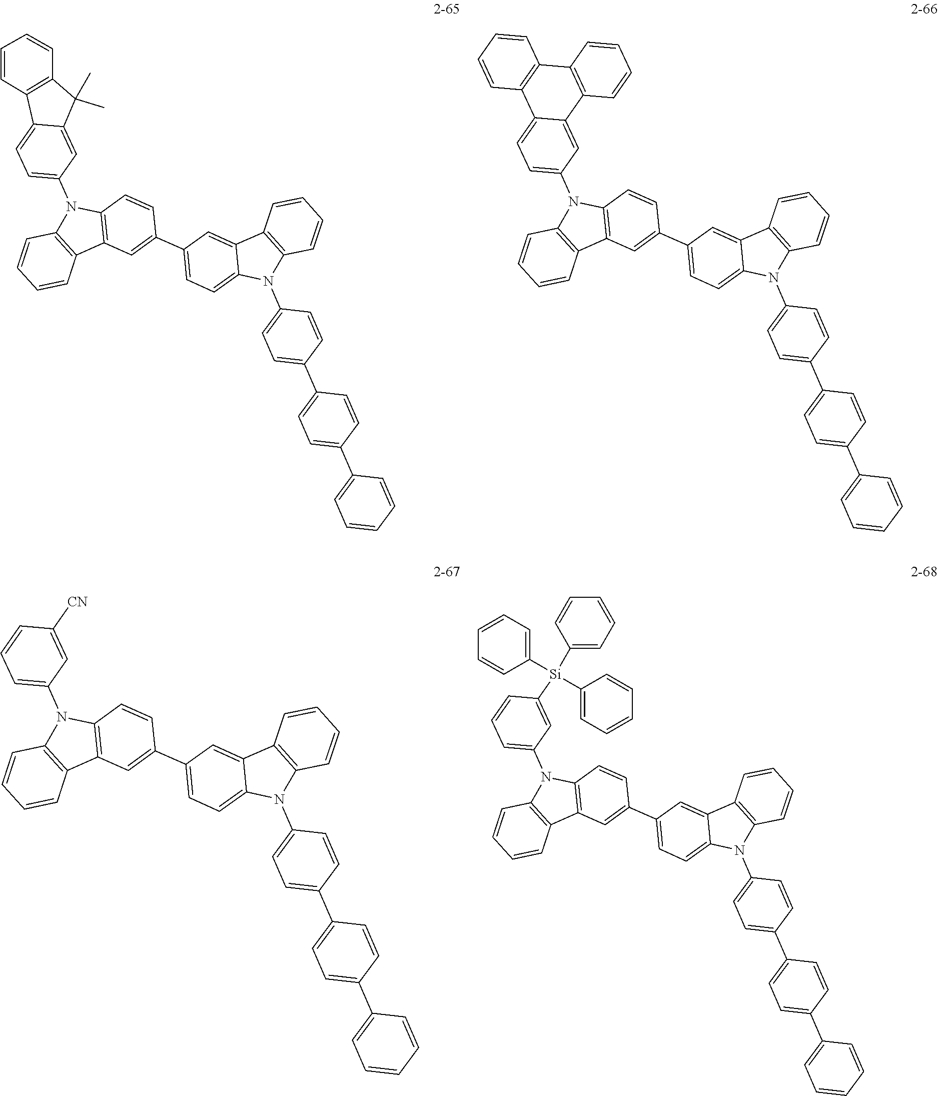 [SMILES: CC1(C)c2ccccc2-c2ccc(-n3c4ccccc4c4cc(-c5ccc6c(c5)c5ccccc5n6-c5ccc(-c6ccc(-c7ccccc7)cc6)cc5)ccc43)cc21 ; c1ccc(-c2ccc(-c3ccc(-n4c5ccccc5c5cc(-c6ccc7c(c6)c6ccccc6n7-c6ccc7c8ccccc8c8ccccc8c7c6)ccc54)cc3)cc2)cc1 ; N#Cc1cccc(-n2c3ccccc3c3cc(-c4ccc5c(c4)c4ccccc4n5-c4ccc(-c5ccc(-c6ccccc6)cc5)cc4)ccc32)c1 ; c1ccc(-c2ccc(-c3ccc(-n4c5ccccc5c5cc(-c6ccc7c(c6)c6ccccc6n7-c6cccc([Si](c7ccccc7)(c7ccccc7)c7ccccc7)c6)ccc54)cc3)cc2)cc1]

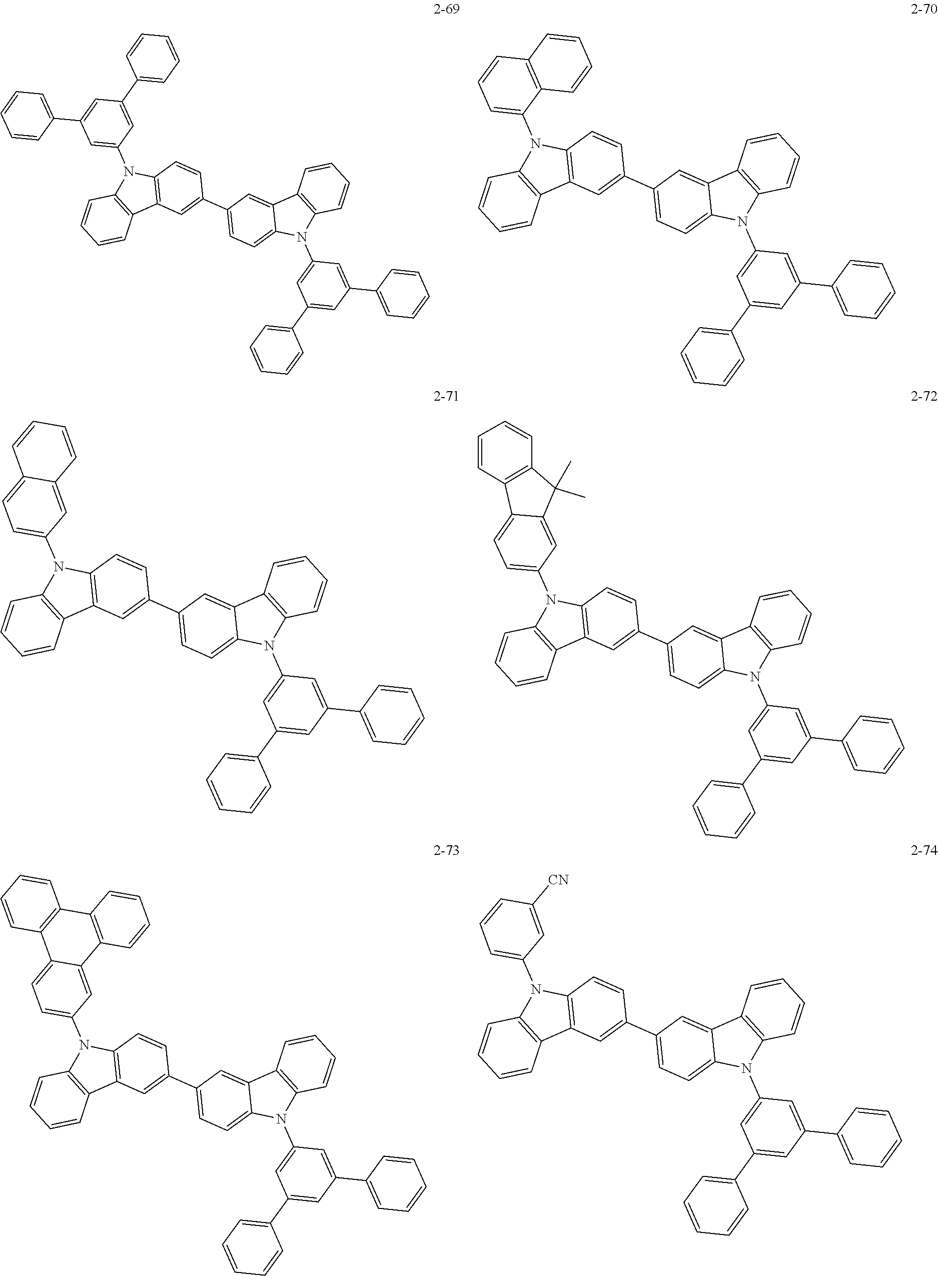

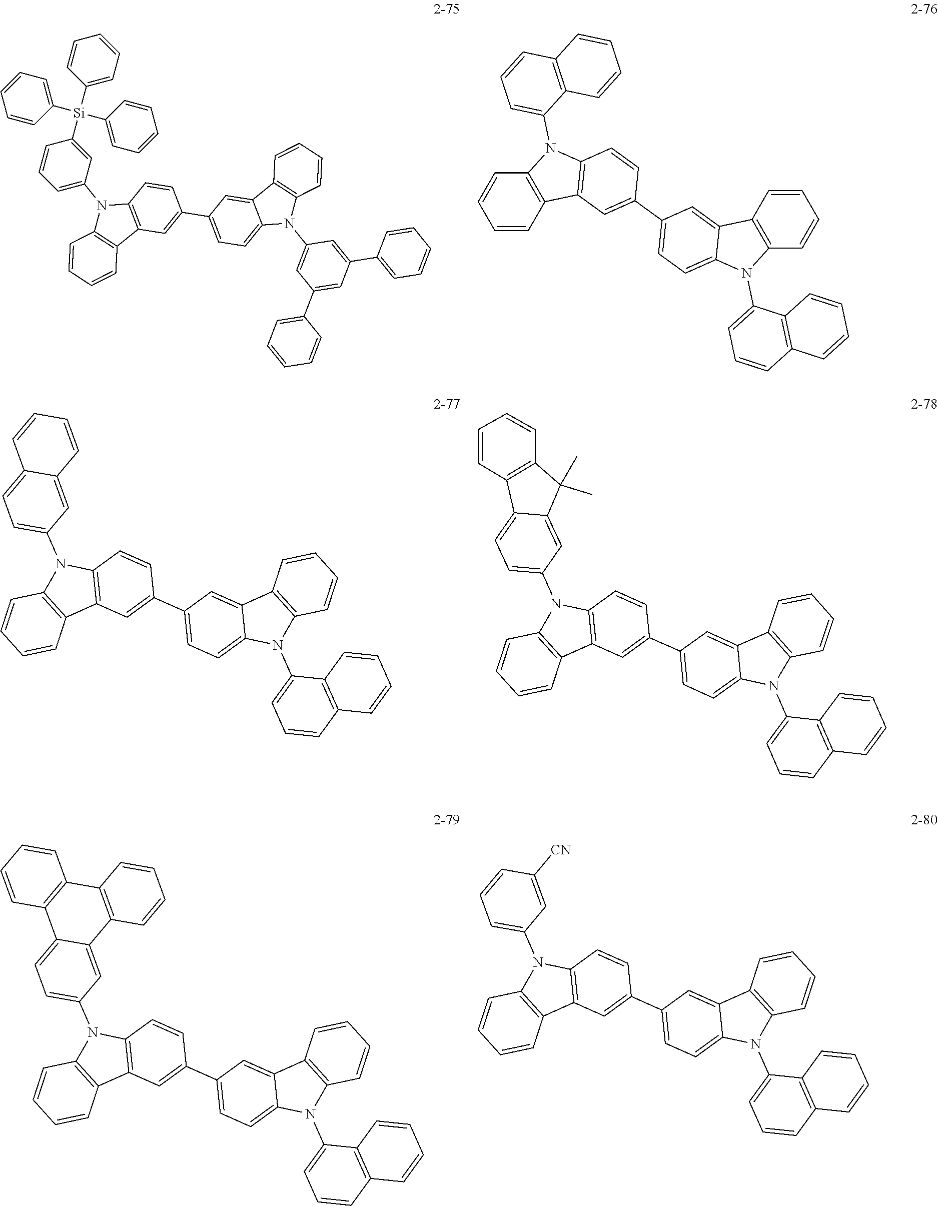

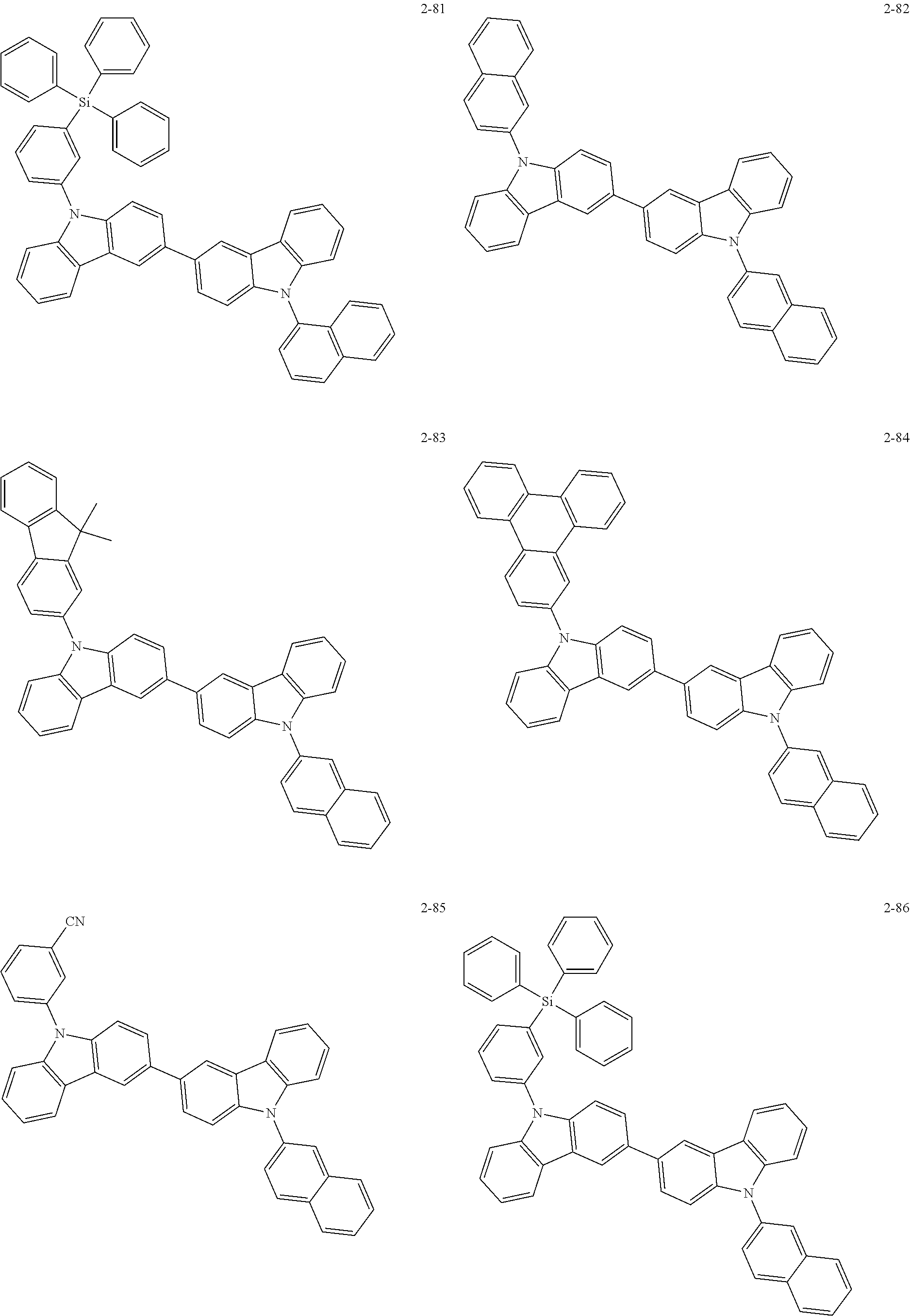

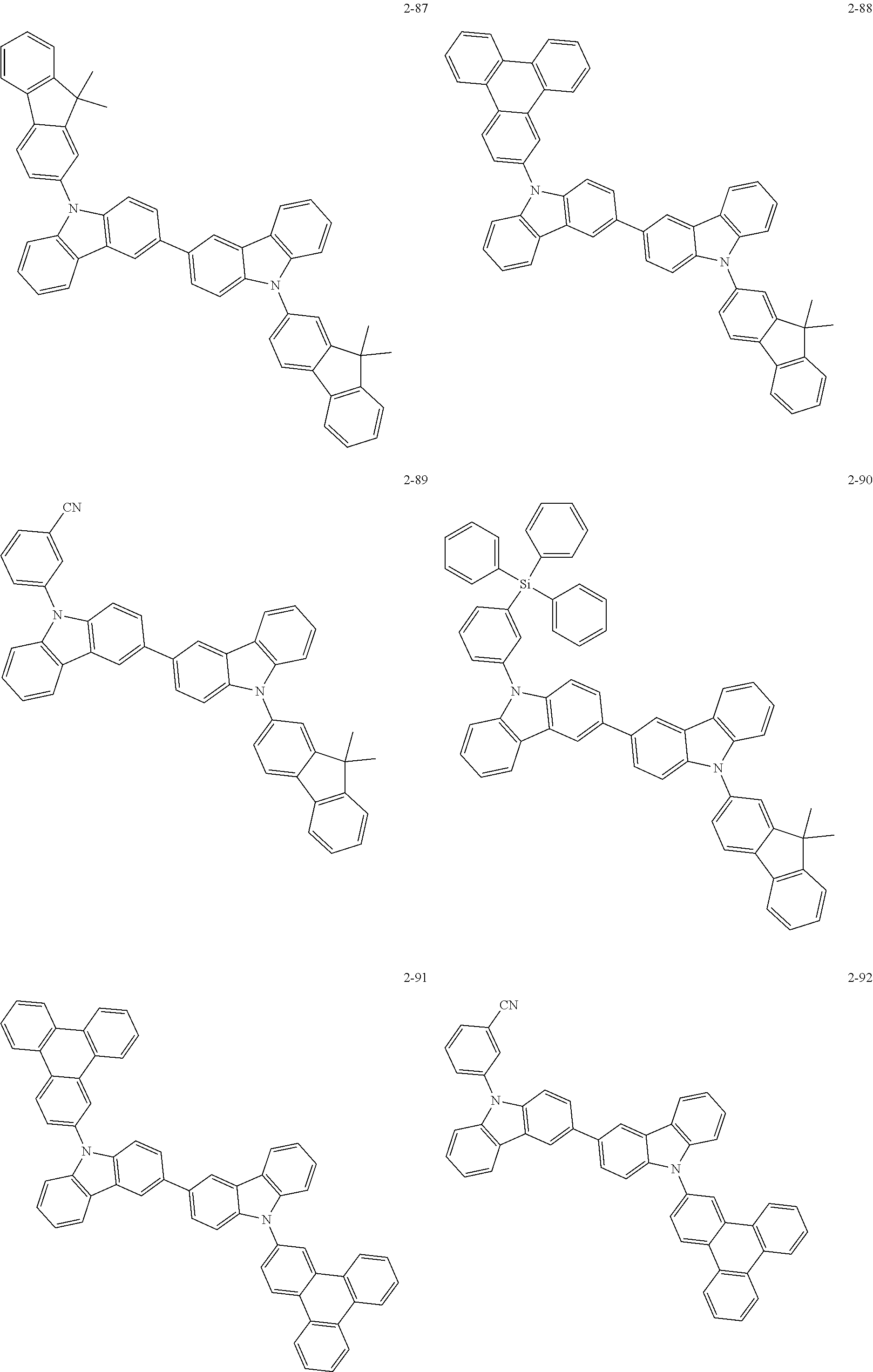

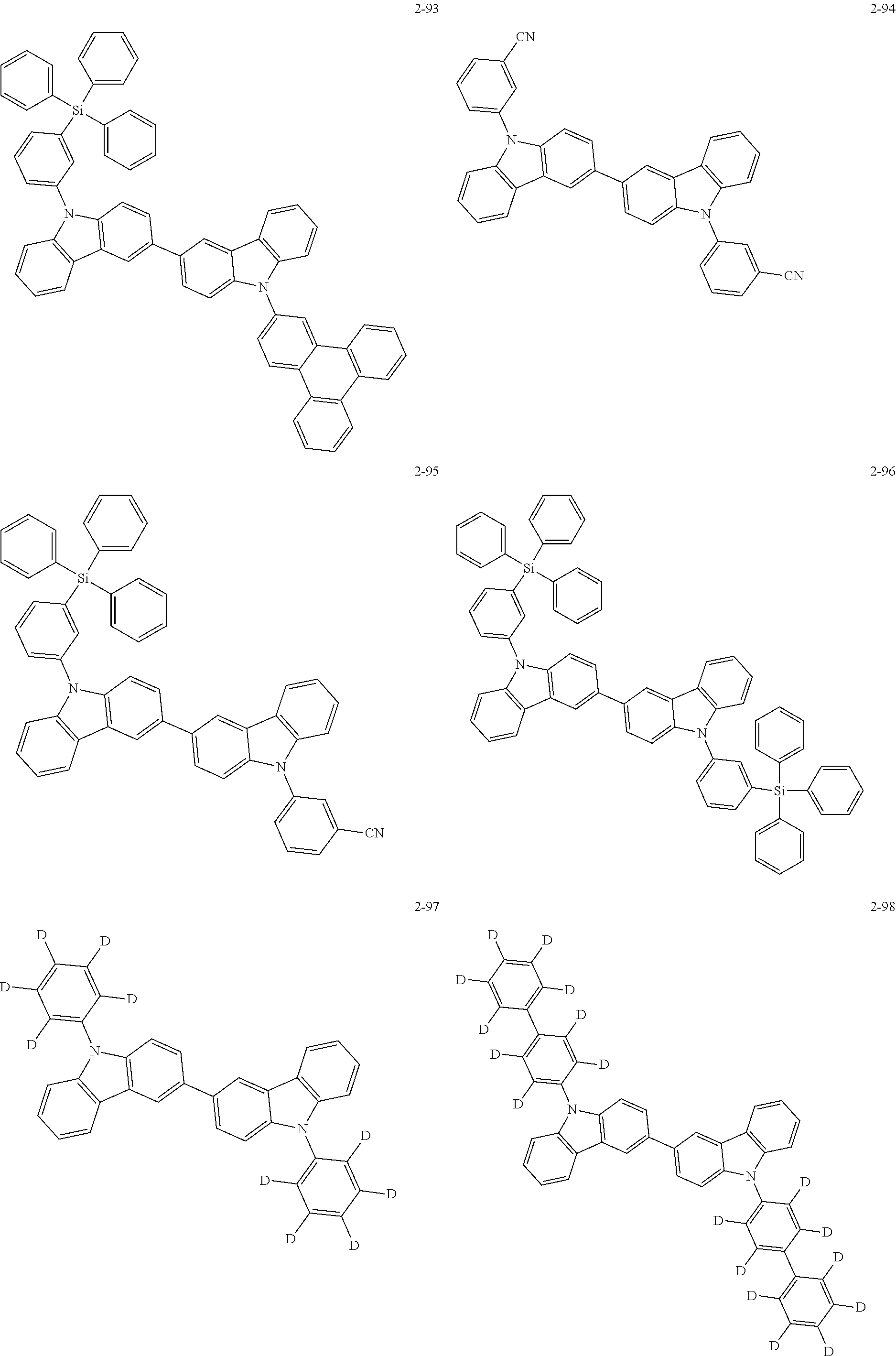

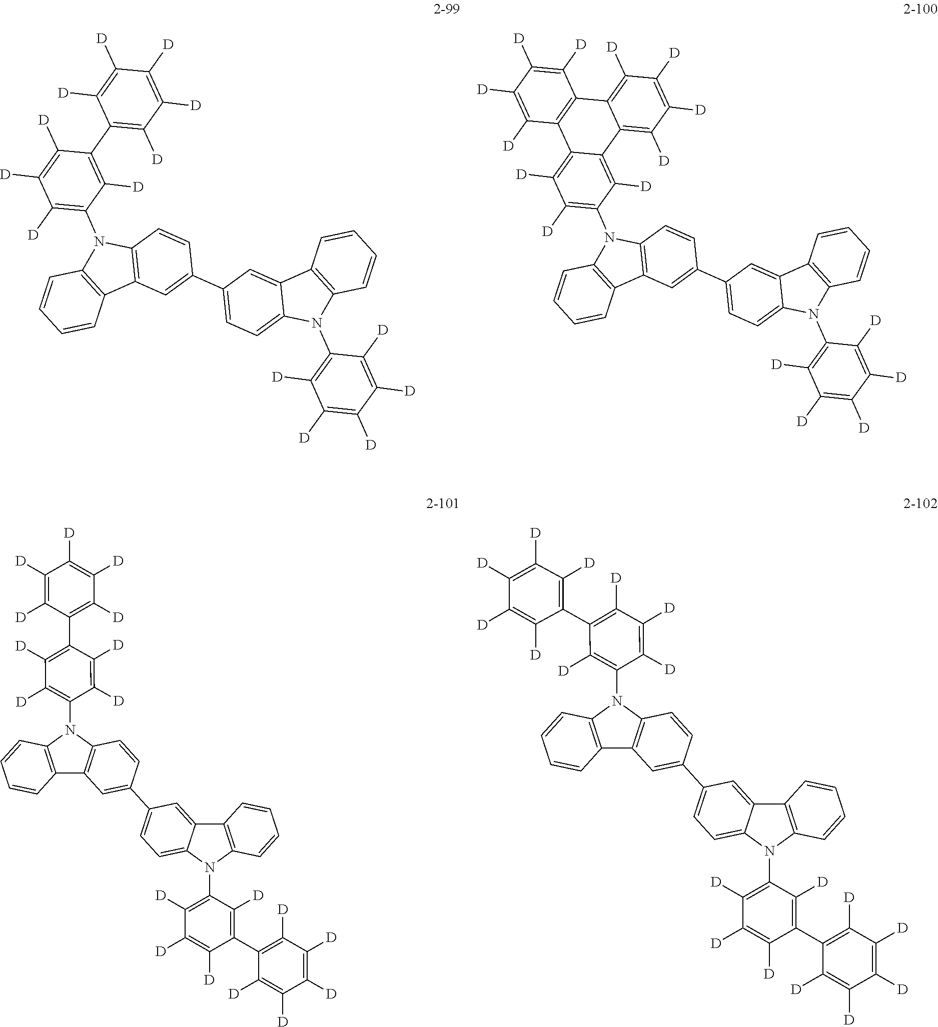

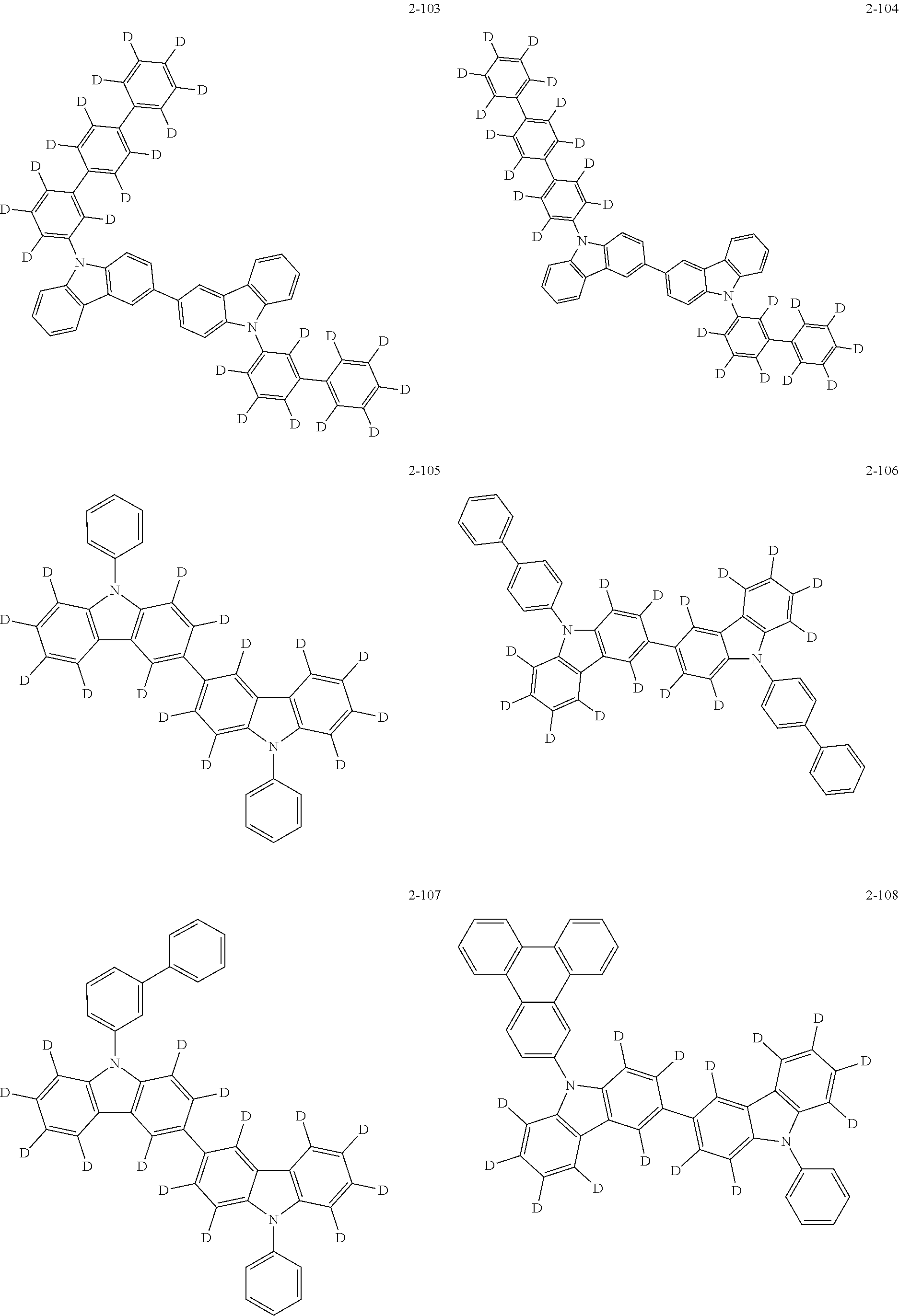

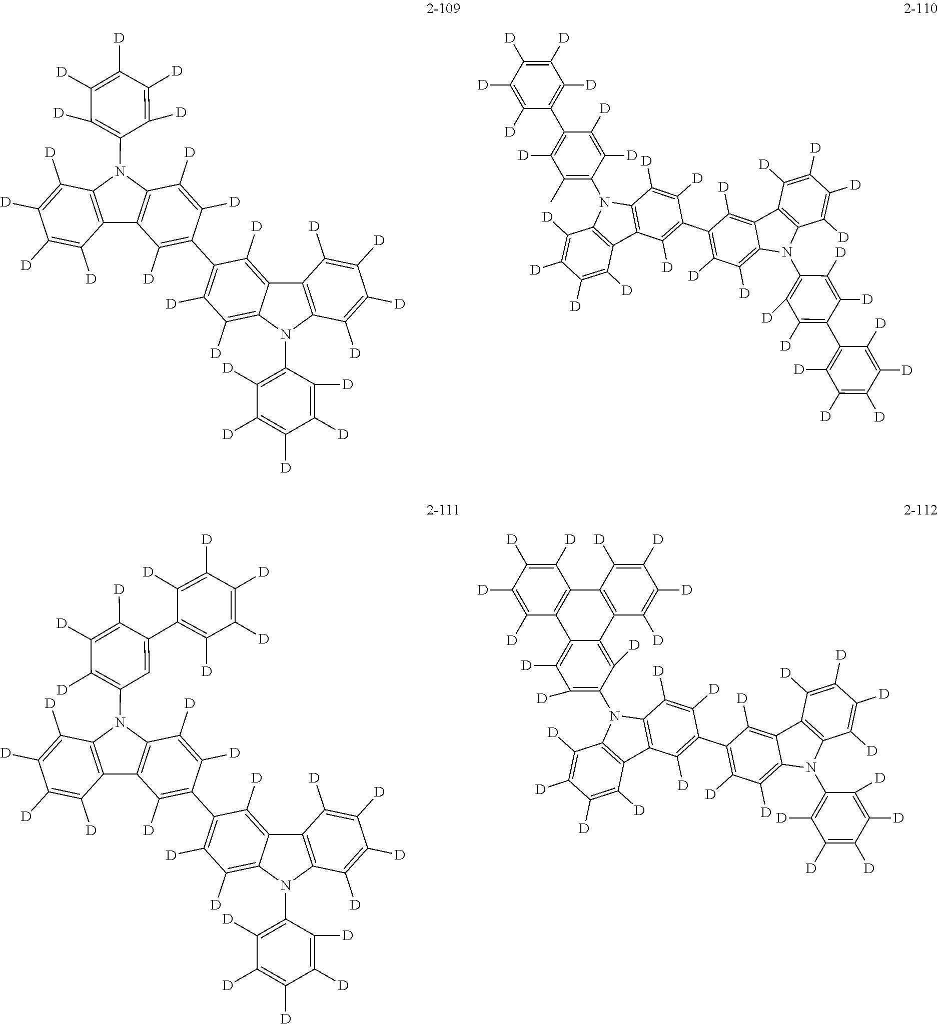

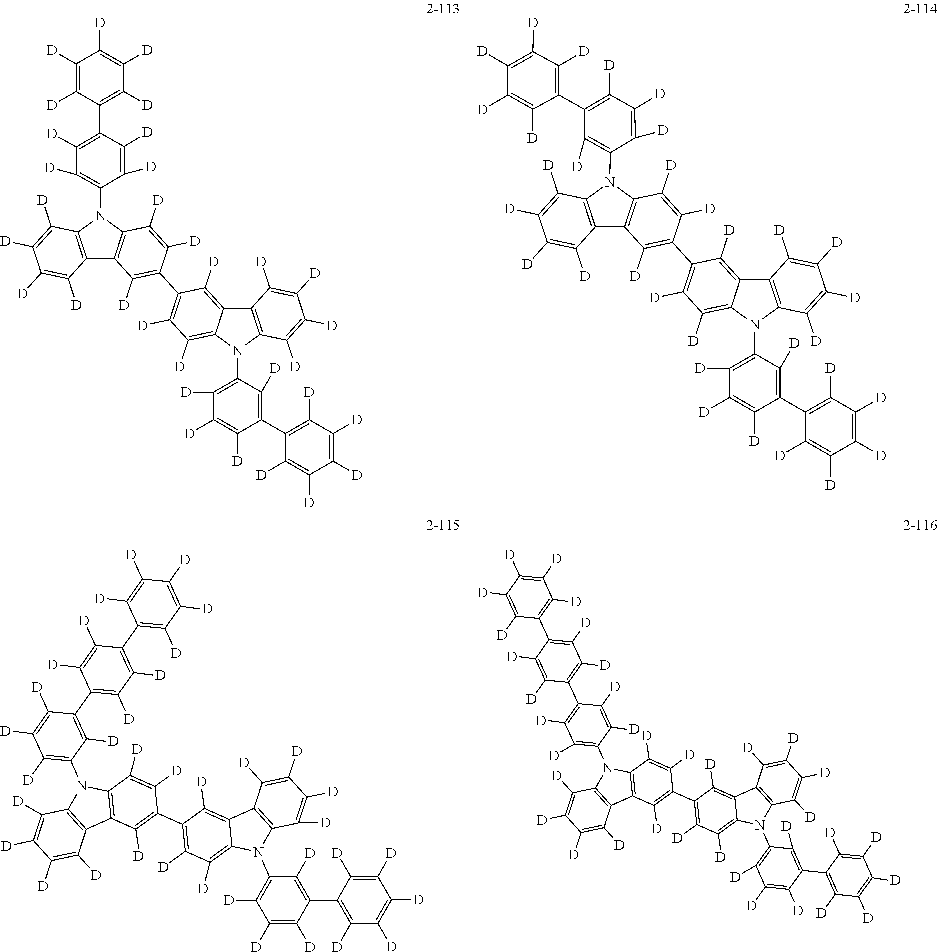

In addition, one embodiment of the present disclosure provides a composition for an organic material layer of an organic light emitting device, the composition comprising: the heterocyclic compound represented by Chemical Formula 1; and the heterocyclic compound represented by Chemical Formula 2.

Specific descriptions on the heterocyclic compound represented by Chemical Formula 1 and the heterocyclic compound represented by Chemical Formula 2 are the same as the descriptions provided above.

In one embodiment of the present disclosure, the heterocyclic compound represented by Chemical Formula 1 and the heterocyclic compound represented by Chemical Formula 2 in the composition for an organic material layer of an organic light emitting device may have a weight ratio of 1:9 to 9:1, 1:9 to 5:5 or 2:8 to 5:5, however, the ratio is not limited thereto.

The composition for an organic material layer of an organic light emitting device may be used when an organic material of an organic light emitting device is formed, and particularly, may be more preferably used when a host of a light emitting layer is formed.

In one embodiment of the present disclosure, the organic material layer includes the heterocyclic compound represented by Chemical Formula 1 and the heterocyclic compound represented by Chemical Formula 2, and a phosphorescent dopant may be used therewith.

As the phosphorescent dopant material, those known in the art may be used. For example, phosphorescent dopant materials represented by LL′MX′, LL′L″M, LMX′X″, L₂MX′ and L₃M may be used, however, the scope of the present disclosure is not limited by these examples.
M may be iridium, platinum, osmium or the like.
L is an anionic bidentate ligand coordinated to M by sp² carbon and heteroatom, and X may function to trap electrons or holes. Nonlimiting examples of L may include 2-(1-naphthyl) benzoxazole, (2-phenylbenzoxazole), (2-phenylbenzothiazole), (7,8-benzoquinoline), phenylpyridine, benzothiophenylpyrizine, 3-methoxy-2-phenylpyridine, thiophenylpyrizine, tolylpyridine and the like. Nonlimiting examples of X′ and X″ may include acetylacetonate (acac), hexafluoroacetylacetonate, salicylidene, picolinate, 8-hydroxyquinolinate and the like.

Specific examples of the phosphorescent dopant are shown below, however, the phosphorescent dopant is not limited to these examples.

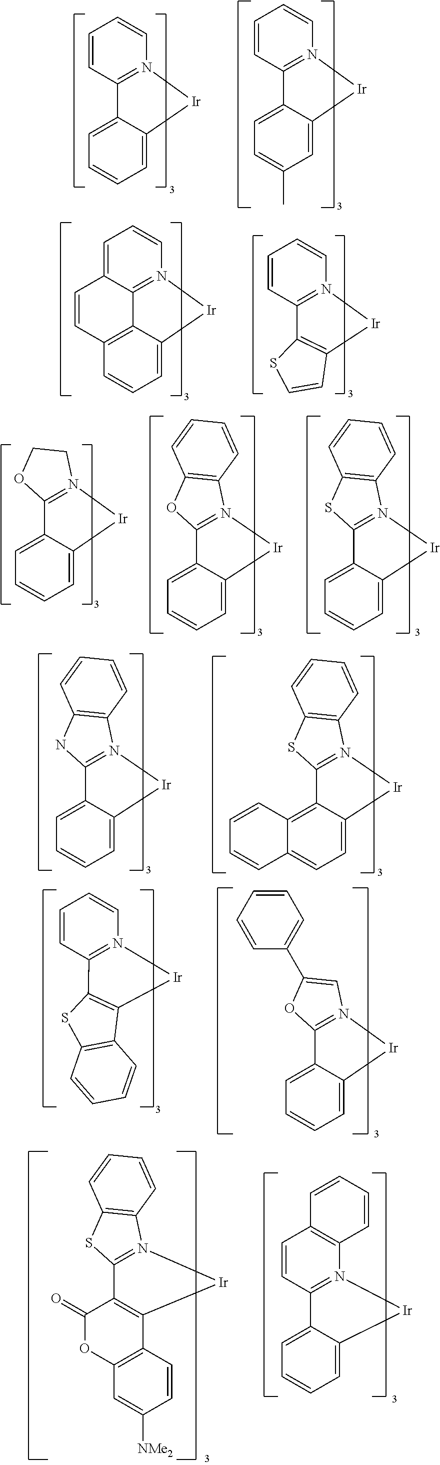

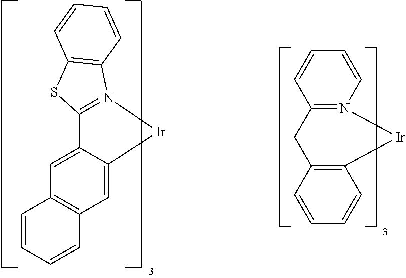

In one embodiment of the present disclosure, the organic material layer includes the heterocyclic compound represented by Chemical Formula 1 and the heterocyclic compound represented by Chemical Formula 2, and an iridium-based dopant may be used therewith.

In one embodiment of the present disclosure, as the iridium-based dopant, (piq)₂(Ir)(acac) may be used as a red phosphorescent dopant or Ir(ppy)₃ may be used as a green phosphorescent dopant.

In one embodiment of the present disclosure, a content of the dopant may be from 1% by weight to 15% by weight, preferably from 2% by weight to 10% by weight and more preferably from 3% by weight to 7% by weight based on the total weight of the light emitting layer.

In the organic light emitting device according to one embodiment of the present disclosure, the organic material layer includes an electron injection layer or an electron transport layer, and the electron injection layer or the electron transport layer may include the heterocyclic compound represented by Chemical Formula 1.

In the organic light emitting device according to another embodiment of the present disclosure, the organic material layer includes an electron blocking layer or a hole blocking layer, and the electron blocking layer or the hole blocking layer may include the heterocyclic compound represented by Chemical Formula 1.

In the organic light emitting device according to another embodiment, the organic material layer includes an electron transport layer, a light emitting layer or a hole blocking layer, and the electron transport layer, the light emitting layer or the hole blocking layer may include the heterocyclic compound represented by Chemical Formula 1.

In the organic light emitting device according to another embodiment, the organic material layer includes a light emitting layer, and the light emitting layer may include the heterocyclic compound represented by Chemical Formula 1.

In the organic light emitting device according to another embodiment, the organic material layer includes a light emitting layer, the light emitting layer includes a host material, and the host material may include the heterocyclic compound represented by Chemical Formula 1.

In the organic light emitting device according to another embodiment, the light emitting layer may include two or more host materials, and at least one of the host materials may include the heterocyclic compound represented by Chemical Formula 1, and the other one may include the heterocyclic compound represented by Chemical Formula 2.

In the organic light emitting device according to another embodiment, two or more host materials may be pre-mixed and used in the light emitting layer, and at least one of the two or more host materials may include the heterocyclic compound represented by Chemical Formula 1, and the other one may include the heterocyclic compound represented by Chemical Formula 2.

The pre-mixing means, before depositing the two or more host materials on the organic material layer, mixing the materials first in one source of supply.

The organic light emitting device according to one embodiment of the present disclosure may further include one, two or more layers selected from the group consisting of a light emitting layer, a hole injection layer, a hole transport layer, an electron injection layer, an electron transport layer, an electron blocking layer and a hole blocking layer.

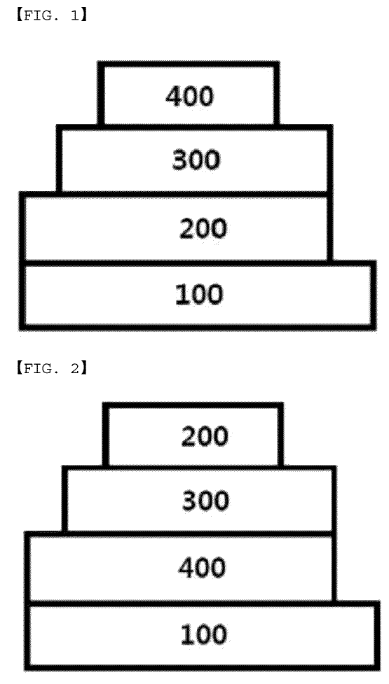

FIG. 1 to FIG. 4 illustrate a lamination order of electrodes and organic material layers of the organic light emitting device according to one embodiment of the present disclosure. However, the scope of the present application is not limited by these drawings, and structures of organic light emitting devices known in the art may also be applied to the present application.

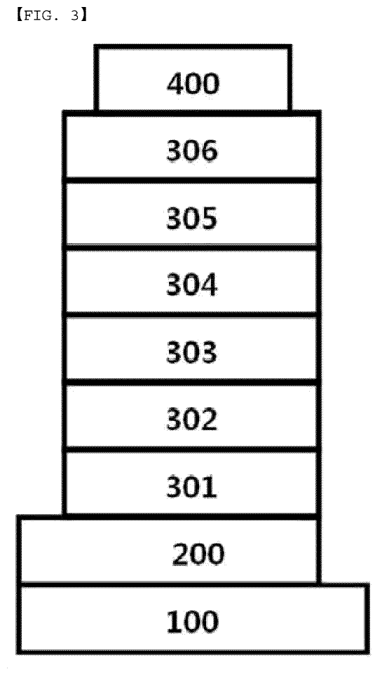

FIG. 1 illustrates an organic light emitting device in which a positive electrode 200, an organic material layer 300 and a negative electrode 400 are sequentially laminated on a substrate 100. However, the structure of the organic light emitting device is not limited only to such a structure, and as illustrated in FIG. 2, an organic light emitting device in which a negative electrode, an organic material layer and a positive electrode are sequentially laminated on a substrate may also be obtained.

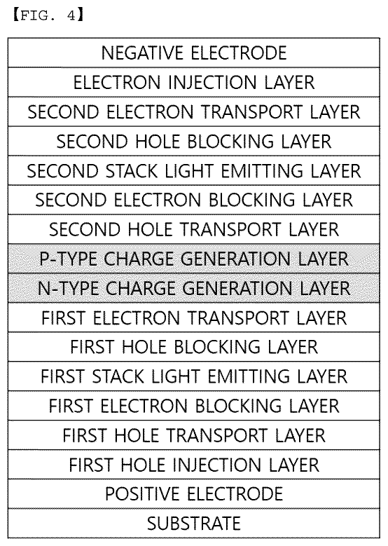

FIG. 3 illustrates a case of the organic material layer being a multilayer. An organic light emitting device according to FIG. 3 includes a hole injection layer 301, a hole transport layer 302, a light emitting layer 303, a hole blocking layer 304, an electron transport layer 305 and an electron injection layer 306. However, the scope of the present application is not limited by such a lamination structure, and as necessary, the layers other than the light emitting layer may not be included, and other necessary functional layers may be further added.

As the organic light emitting device according to one embodiment of the present application, an organic light emitting device having a 2-stack tandem structure is schematically illustrated in FIG. 4.

Herein, a first electron blocking layer, a first hole blocking layer, a second hole blocking layer and the like described in FIG. 4 may not be included in some cases.

One embodiment of the present disclosure provides a method for manufacturing an organic light emitting device, the method including: preparing a substrate; forming a first electrode on the substrate; forming one or more organic material layers on the first electrode; and forming a second electrode on the one or more organic material layers, wherein the forming of one or more organic material layers includes forming the one or more organic material layers using the composition for an organic material layer according to one embodiment of the present disclosure.

In one embodiment of the present disclosure, the forming of organic material layers may be forming organic material layers using a thermal vacuum deposition method after pre-mixing the heterocyclic compound represented by Chemical Formula 1 and the heterocyclic compound represented by Chemical Formula 2.

The pre-mixing means, before depositing the heterocyclic compound represented by Chemical Formula 1 and the heterocyclic compound represented by Chemical Formula 2 on the organic material layer, mixing the materials first in one source of supply.

The pre-mixed material may be referred to as the composition for an organic material layer according to one embodiment of the present application.

The organic material layer including the heterocyclic compound represented by Chemical Formula 1 may further include other materials as necessary.

The organic material layer including the heterocyclic compound represented by Chemical Formula 1 and the heterocyclic compound represented by Chemical Formula 2 at the same time may further include other materials as necessary.

In the organic light emitting device according to one embodiment of the present disclosure, materials other than the heterocyclic compound represented by Chemical Formula 1 or the heterocyclic compound represented by Chemical Formula 2 are illustrated below, however, these are for illustrative purposes only and not for limiting the scope of the present application, and these materials may be replaced by materials known in the art.

As the positive electrode material, materials each having a relatively large work function may be used, and transparent conductive oxides, metals, conductive polymers or the like may be used. Specific examples of the positive electrode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the negative electrode material, materials each having a relatively small work function may be used, and metals, metal oxides, conductive polymers or the like may be used. Specific examples of the negative electrode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection layer material, known hole injection layer materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], conductive polymers having solubility such as polyaniline/dodecylbenzenesulfonic acid or poly(3,4-ethylenedioxythiophene)/poly(4-styrenesulfonate), polyaniline/camphor sulfonic acid or polyaniline/poly(4-styrenesulfonate), and the like, may be used.

As the hole transport layer material, pyrazoline derivatives, arylamine-based derivatives, stilbene derivatives, triphenyldiamine derivatives and the like may be used, and low molecular or high molecular materials may also be used.

As the electron transport layer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials as well as low molecular materials may also be used.

As examples of the electron injection layer material, LiF is typically used in the art, however, the present application is not limited thereto.

As the light emitting layer material, red, green or blue light emitting materials may be used, and as necessary, two or more light emitting materials may be mixed and used. Herein, the two or more light emitting materials may be deposited as individual sources of supply or pre-mixed and deposited as one source of supply when used. In addition, fluorescent materials may also be used as the light emitting layer material, however, phosphorescent materials may also be used. As the light emitting layer material, materials emitting light alone by binding holes and electrons injected from a positive electrode and a negative electrode, respectively, may be used, however, materials having a host material and a dopant material involving together in light emission may also be used.

When hosts of the light emitting layer material are mixed and used, same series hosts may be mixed and used, or different series hosts may be mixed and used. For example, any two or more types of materials among n-type host materials and p-type host materials may be selected and used as a host material of a light emitting layer.

The organic light emitting device according to one embodiment of the present disclosure may be a top-emission type, a bottom-emission type or a dual-emission type depending on the materials used.

The heterocyclic compound according to one embodiment of the present disclosure may also be used in an organic electronic device including an organic solar cell, an organic photo conductor, an organic transistor and the like under a principle similar to that in the organic light emitting device.

Hereinafter, preferred examples are provided to help to understand t disclosure, however, the following examples are only provided to more readily understand the present disclosure, and the present disclosure is not limited thereto.

### PREPARATION EXAMPLE

### Preparation Example 1. Preparation of Compound 1-1

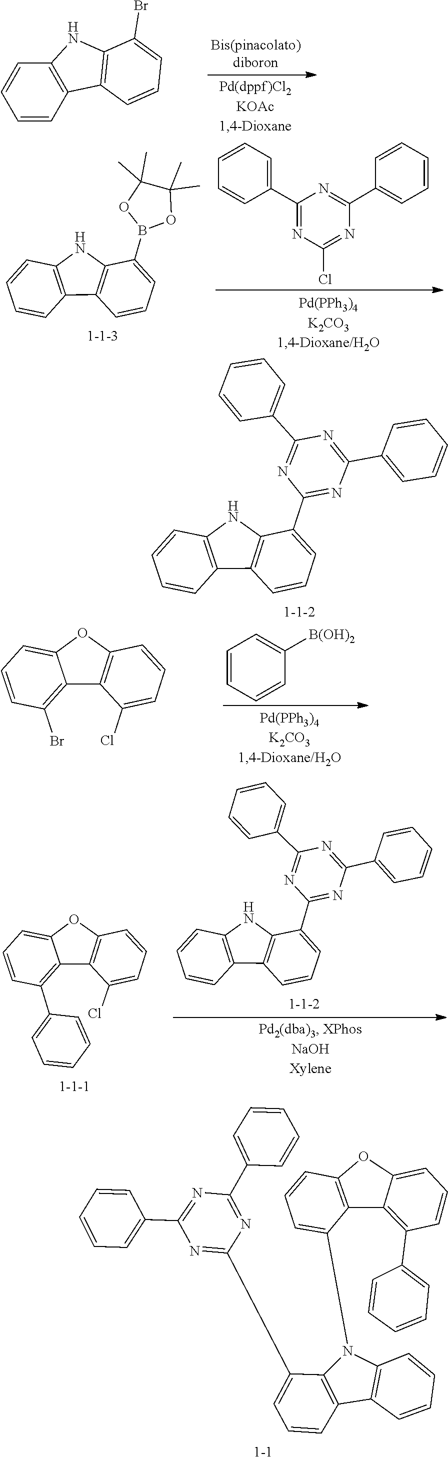

### Preparation Example 1-1. Preparation of Compound 1-1-3

1-bromo-9H-carbazole (10 g, 40.6 mM), bis(pinacolato)diboron (20.6 g, 81.2 mM), Pd(dppf)Cl₂ (1.48 g, 2.03 mM) and potassium acetate (KOAc) (11.9 g, 121.8 mM) were dissolved in 1,4-dioxane (100 mL), and then the mixture was refluxed for 4 hours at 110° C.

After the reaction was completed, the result was extracted by introducing distilled water and dichloromethane (DCM) thereto at room temperature, and after drying the organic layer with MgSO₄, the solvent was removed using a rotary evaporator.

The reaction material was purified by column chromatography (dichloromethane:hexane=1:2 (volume ratio)) to obtain target Compound 1-1-3 (10.5 g, yield 88%).

### Preparation Example 1-2. Preparation of Compound 1-1-2

Compound 1-1-3 (10.5 g, 35.8 mM), 2-chloro-4, 6-diphenyl-1, 3, 5-triazine (9.5 g, 35.8 mM), tetrakis(triphenylhosphine)palladium(0) (Pd(PPh₃)₄) (2.0 g, 1.8 m M) and K₂CO₃ (14.8 g, 45 mM) were dissolved in 1,4-dioxane (100 mL) and water (20 mL), and then the mixture was refluxed for 5 hours at 110° C.

After the reaction was completed, the result was extracted by introducing distilled water and dichloromethane (DCM) thereto at room temperature, and after drying the organic layer with MgSO₄, the solvent was removed using a rotary evaporator.

The reaction material was purified by column chromatography (dichloromethane:hexane=1:2 (volume ratio)) to obtain target Compound 1-1-2 (12.3 g, yield 86%).

### Preparation Example 1-3. Preparation of Compound 1-1-1

1-bromo-9-chlorodibenzo[b,d]furan (10 g, 35.5 mM), phenylboronic acid (8.6 g, 71.0 mM), (tetrakis(triphenylhosphine)palladium(0) (Pd(PPh₃)₄) (2.0 g, 1.77 mM) and K₂CO₃ (14.7 g, 106.5 mM) were dissolved in 1,4-dioxane (100 mL) and water (20 mL), and then the mixture was refluxed for 4 hours at 110° C.

After the reaction was completed, the result was extracted by introducing distilled water and dichloromethane (DCM) thereto at room temperature, and after drying the organic layer with MgSO₄, the solvent was removed using a rotary evaporator.

The reaction material was purified by column chromatography (dichloromethane:hexane=1:3 (volume ratio)) to obtain target Compound 1-1-1 (9.2 g, yield 93%).

### Preparation Example 1-4. Preparation of Compound 1-1

Compound 1-1-1 (9.2 g, 33.0 mM), Compound 1-1-2 (13.1 g, 33.0 mM), tris(dibenzylideneacetone)dipalladium (Pd₂(dba)₃) (3.0 g, 3.3 mM), dicyclohexyl(2′,4′,6′-triisopropyl-[1,1′-biphenyl]-2-yl) phosphine (Xphos) (4.7 g, 9.9 mM) and sodium hydroxide (NaOH) (3.3 g, 82.5 mM) were dissolved in xylene (100 mL), and then the mixture was refluxed for 12 hours at 140° C.

After the reaction was completed, the result was extracted by introducing distilled water and dichloromethane (DCM) thereto at room temperature, and after drying the organic layer with MgSO₄, the solvent was removed using a rotary evaporator.

The reaction material was purified by column chromatography (dichloromethane:hexane=1:3 (volume ratio)), and recrystallized with methanol to obtain target Compound 1-1 (12.5 g, yield 64%).

Target compounds were synthesized in the same manner as in Preparation Example 1, except that Intermediate A of the following Table 1 was used instead of 1-bromo-9H-carbazole, Intermediate B of the following Table 1 was used instead of 2-chloro-4,6-diphenyl-1,3,5-triazine, Intermediate C of the following Table 1 was used instead of 1-bromo-9-chlorodibenzo[b,d]furan, and Intermediate D of the following Table 1 was used instead of phenylboronic acid.

TABLE 1

| Intermedi- | Intermedi- | Intermedi- |
|---|---|---|
| ate A | ate B | ate C |
| 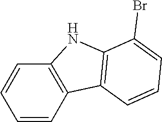 [SMILES: Brc1cccc2c1[nH]c1ccccc12] | 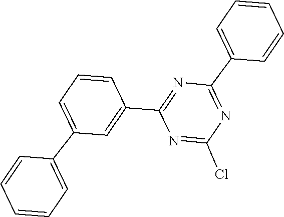 [SMILES: Clc1nc(-c2ccccc2)nc(-c2cccc(-c3ccccc3)c2)n1] | 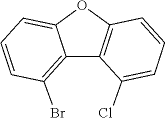 [SMILES: Clc1cccc2oc3cccc(Br)c3c12] |
| 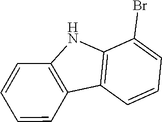 [SMILES: Brc1cccc2c1[nH]c1ccccc12] | 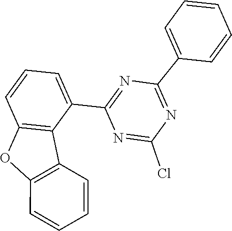 [SMILES: Clc1nc(-c2ccccc2)nc(-c2cccc3oc4ccccc4c23)n1] | 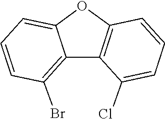 [SMILES: Clc1cccc2oc3cccc(Br)c3c12] |
| 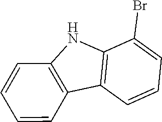 [SMILES: Brc1cccc2c1[nH]c1ccccc12] | 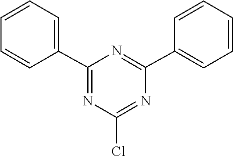 [SMILES: Clc1nc(-c2ccccc2)nc(-c2ccccc2)n1] | 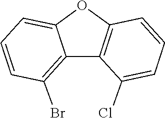 [SMILES: Clc1cccc2oc3cccc(Br)c3c12] |
| 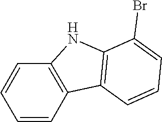 [SMILES: Brc1cccc2c1[nH]c1ccccc12] | 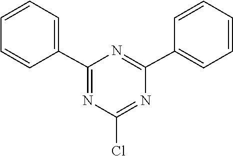 [SMILES: Clc1nc(-c2ccccc2)nc(-c2ccccc2)n1] | 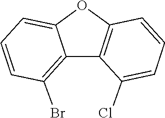 [SMILES: Clc1cccc2oc3cccc(Br)c3c12] |
| 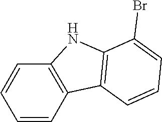 [SMILES: Brc1cccc2c1[nH]c1ccccc12] | 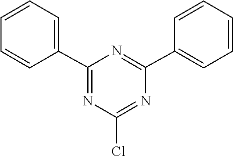 [SMILES: Clc1nc(-c2ccccc2)nc(-c2ccccc2)n1] | 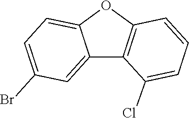 [SMILES: Clc1cccc2oc3ccc(Br)cc3c12] |
| 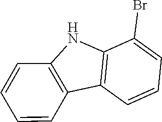 [SMILES: Brc1cccc2c1[nH]c1ccccc12] | 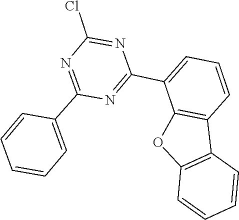 [SMILES: Clc1nc(-c2ccccc2)nc(-c2cccc3c2oc2ccccc23)n1] | 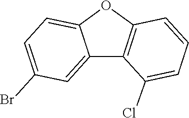 [SMILES: Clc1cccc2oc3ccc(Br)cc3c12] |
| 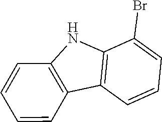 [SMILES: Brc1cccc2c1[nH]c1ccccc12] | 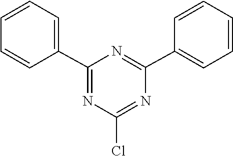 [SMILES: Clc1nc(-c2ccccc2)nc(-c2ccccc2)n1] | 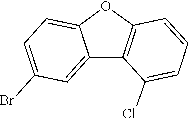 [SMILES: Clc1cccc2oc3ccc(Br)cc3c12] |
| 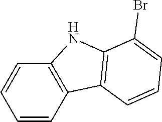 [SMILES: Brc1cccc2c1[nH]c1ccccc12] | 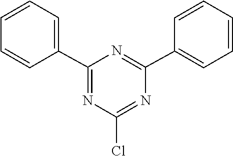 [SMILES: Clc1nc(-c2ccccc2)nc(-c2ccccc2)n1] | 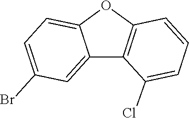 [SMILES: Clc1cccc2oc3ccc(Br)cc3c12] |
| 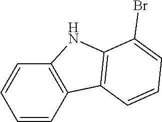 [SMILES: Brc1cccc2c1[nH]c1ccccc12] | 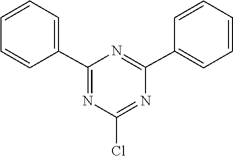 [SMILES: Clc1nc(-c2ccccc2)nc(-c2ccccc2)n1] | 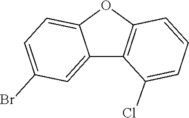 [SMILES: Clc1cccc2oc3ccc(Br)cc3c12] |
| 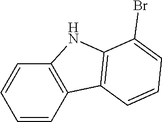 [SMILES: Brc1cccc2c1[nH]c1ccccc12] | 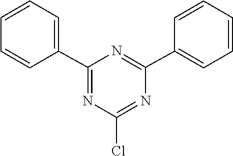 [SMILES: Clc1nc(-c2ccccc2)nc(-c2ccccc2)n1] | 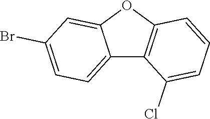 [SMILES: Clc1cccc2oc3cc(Br)ccc3c12] |
| 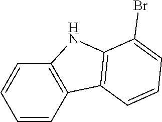 [SMILES: Brc1cccc2c1[nH]c1ccccc12] | 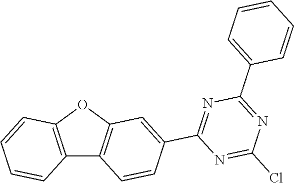 [SMILES: Clc1nc(-c2ccccc2)nc(-c2ccc3c(c2)oc2ccccc23)n1] | 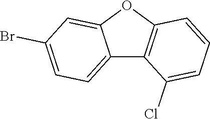 [SMILES: Clc1cccc2oc3cc(Br)ccc3c12] |
| 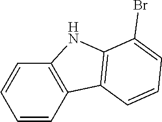 [SMILES: Brc1cccc2c1[nH]c1ccccc12] | 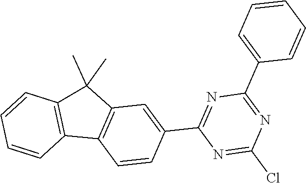 [SMILES: CC1(C)c2ccccc2-c2ccc(-c3nc(Cl)nc(-c4ccccc4)n3)cc21] | 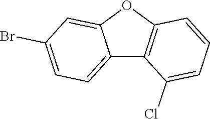 [SMILES: Clc1cccc2oc3cc(Br)ccc3c12] |
| 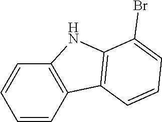 [SMILES: Brc1cccc2c1[nH]c1ccccc12] | 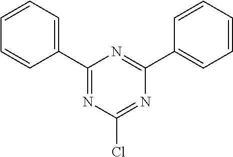 [SMILES: Clc1nc(-c2ccccc2)nc(-c2ccccc2)n1] | 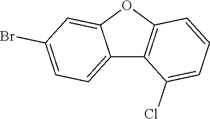 [SMILES: Clc1cccc2oc3cc(Br)ccc3c12] |
| 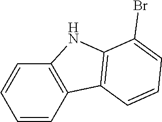 [SMILES: Brc1cccc2c1[nH]c1ccccc12] | 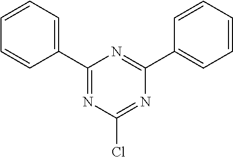 [SMILES: Clc1nc(-c2ccccc2)nc(-c2ccccc2)n1] | 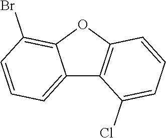 [SMILES: Clc1cccc2oc3c(Br)cccc3c12] |
| 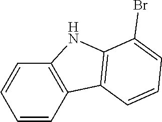 [SMILES: Brc1cccc2c1[nH]c1ccccc12] | 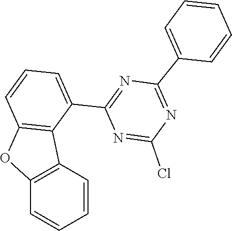 [SMILES: Clc1nc(-c2ccccc2)nc(-c2cccc3oc4ccccc4c23)n1] | 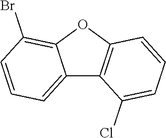 [SMILES: Clc1cccc2oc3c(Br)cccc3c12] |
| 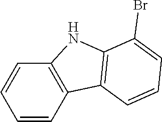 [SMILES: Brc1cccc2c1[nH]c1ccccc12] | 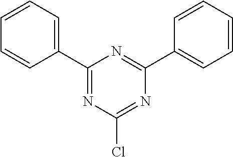 [SMILES: Clc1nc(-c2ccccc2)nc(-c2ccccc2)n1] | 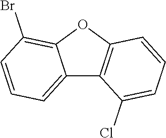 [SMILES: Clc1cccc2oc3c(Br)cccc3c12] |
| 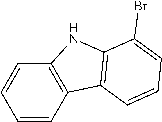 [SMILES: Brc1cccc2c1[nH]c1ccccc12] | 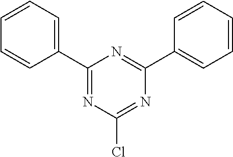 [SMILES: Clc1nc(-c2ccccc2)nc(-c2ccccc2)n1] | 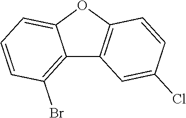 [SMILES: Clc1ccc2oc3cccc(Br)c3c2c1] |
| 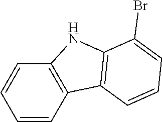 [SMILES: Brc1cccc2c1[nH]c1ccccc12] | 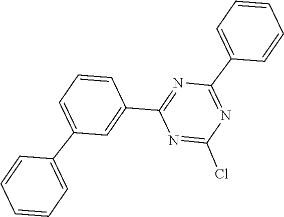 [SMILES: Clc1nc(-c2ccccc2)nc(-c2cccc(-c3ccccc3)c2)n1] | 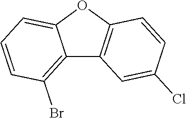 [SMILES: Clc1ccc2oc3cccc(Br)c3c2c1] |
| 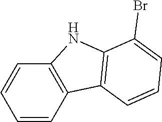 [SMILES: Brc1cccc2c1[nH]c1ccccc12] | 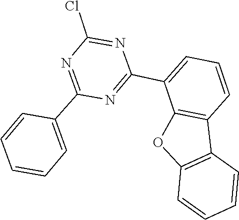 [SMILES: Clc1nc(-c2ccccc2)nc(-c2cccc3c2oc2ccccc23)n1] | 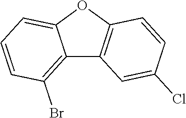 [SMILES: Clc1ccc2oc3cccc(Br)c3c2c1] |
| 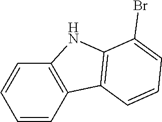 [SMILES: Brc1cccc2c1[nH]c1ccccc12] | 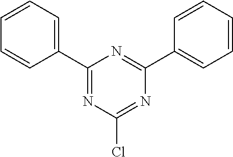 [SMILES: Clc1nc(-c2ccccc2)nc(-c2ccccc2)n1] | 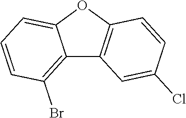 [SMILES: Clc1ccc2oc3cccc(Br)c3c2c1] |
| 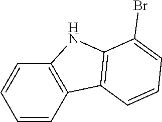 [SMILES: Brc1cccc2c1[nH]c1ccccc12] | 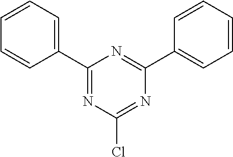 [SMILES: Clc1nc(-c2ccccc2)nc(-c2ccccc2)n1] | 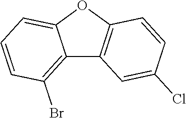 [SMILES: Clc1ccc2oc3cccc(Br)c3c2c1] |
| 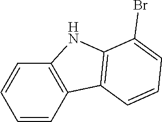 [SMILES: Brc1cccc2c1[nH]c1ccccc12] | 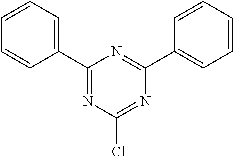 [SMILES: Clc1nc(-c2ccccc2)nc(-c2ccccc2)n1] | 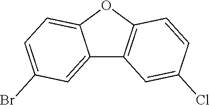 [SMILES: Clc1ccc2oc3ccc(Br)cc3c2c1] |
| 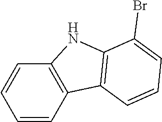 [SMILES: Brc1cccc2c1[nH]c1ccccc12] | 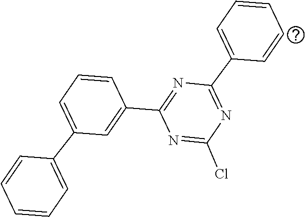 [SMILES: Clc1nc(-c2ccccc2)nc(-c2cccc(-c3ccccc3)c2)n1] | 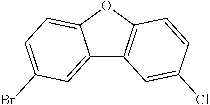 [SMILES: Clc1ccc2oc3ccc(Br)cc3c2c1] |
| 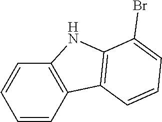 [SMILES: Brc1cccc2c1[nH]c1ccccc12] | 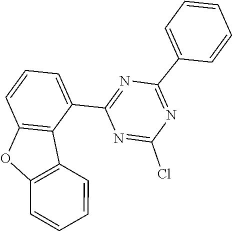 [SMILES: Clc1nc(-c2ccccc2)nc(-c2cccc3oc4ccccc4c23)n1] | 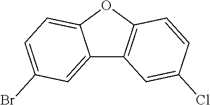 [SMILES: Clc1ccc2oc3ccc(Br)cc3c2c1] |
| 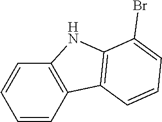 [SMILES: Brc1cccc2c1[nH]c1ccccc12] | 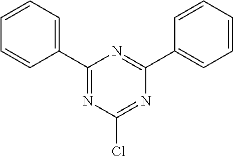 [SMILES: Clc1nc(-c2ccccc2)nc(-c2ccccc2)n1] | 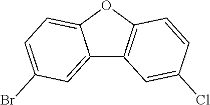 [SMILES: Clc1ccc2oc3ccc(Br)cc3c2c1] |
| 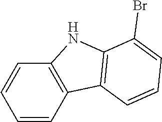 [SMILES: Brc1cccc2c1[nH]c1ccccc12] | 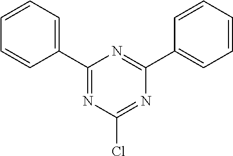 [SMILES: Clc1nc(-c2ccccc2)nc(-c2ccccc2)n1] | 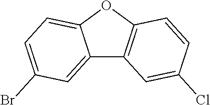 [SMILES: Clc1ccc2oc3ccc(Br)cc3c2c1] |
| 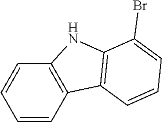 [SMILES: Brc1cccc2c1[nH]c1ccccc12] | 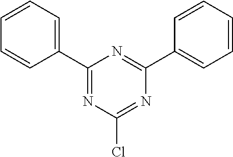 [SMILES: Clc1nc(-c2ccccc2)nc(-c2ccccc2)n1] | 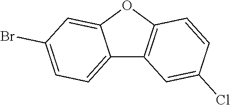 [SMILES: Clc1ccc2oc3cc(Br)ccc3c2c1] |
| 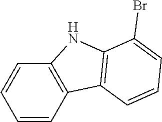 [SMILES: Brc1cccc2c1[nH]c1ccccc12] | 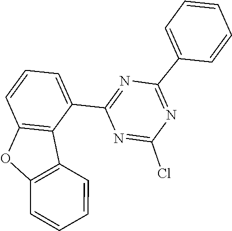 [SMILES: Clc1nc(-c2ccccc2)nc(-c2cccc3oc4ccccc4c23)n1] | 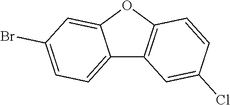 [SMILES: Clc1ccc2oc3cc(Br)ccc3c2c1] |
| 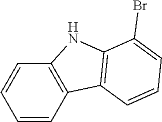 [SMILES: Brc1cccc2c1[nH]c1ccccc12] | 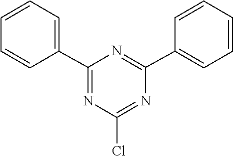 [SMILES: Clc1nc(-c2ccccc2)nc(-c2ccccc2)n1] | 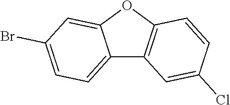 [SMILES: Clc1ccc2oc3cc(Br)ccc3c2c1] |
| 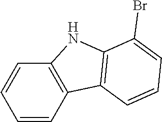 [SMILES: Brc1cccc2c1[nH]c1ccccc12] | 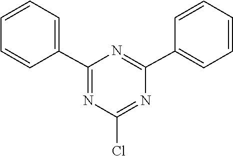 [SMILES: Clc1nc(-c2ccccc2)nc(-c2ccccc2)n1] | 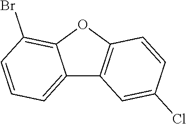 [SMILES: Clc1ccc2oc3c(Br)cccc3c2c1] |
| 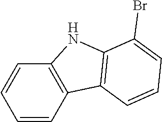 [SMILES: Brc1cccc2c1[nH]c1ccccc12] | 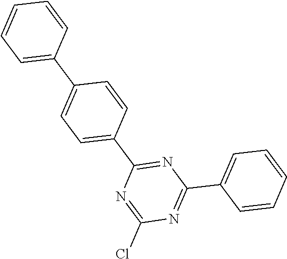 [SMILES: Clc1nc(-c2ccccc2)nc(-c2ccc(-c3ccccc3)cc2)n1] | 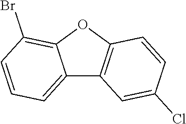 [SMILES: Clc1ccc2oc3c(Br)cccc3c2c1] |
| 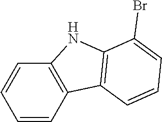 [SMILES: Brc1cccc2c1[nH]c1ccccc12] | 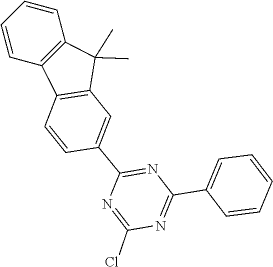 [SMILES: CC1(C)c2ccccc2-c2ccc(-c3nc(Cl)nc(-c4ccccc4)n3)cc21] | 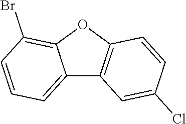 [SMILES: Clc1ccc2oc3c(Br)cccc3c2c1] |
| 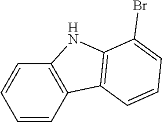 [SMILES: Brc1cccc2c1[nH]c1ccccc12] | 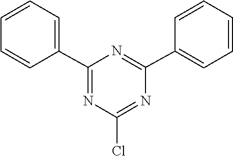 [SMILES: Clc1nc(-c2ccccc2)nc(-c2ccccc2)n1] | 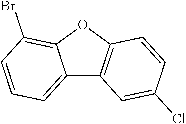 [SMILES: Clc1ccc2oc3c(Br)cccc3c2c1] |
| 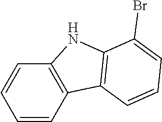 [SMILES: Brc1cccc2c1[nH]c1ccccc12] | 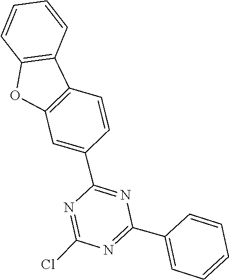 [SMILES: Clc1nc(-c2ccccc2)nc(-c2ccc3c(c2)oc2ccccc23)n1] | 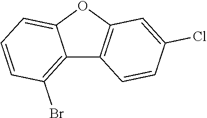 [SMILES: Clc1ccc2c(c1)oc1cccc(Br)c12] |
| 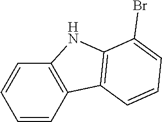 [SMILES: Brc1cccc2c1[nH]c1ccccc12] | 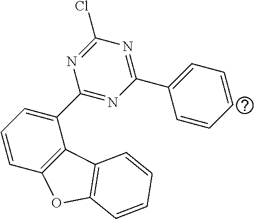 [SMILES: Clc1nc(-c2ccccc2)nc(-c2cccc3oc4ccccc4c23)n1] | 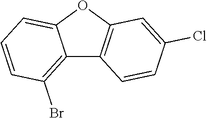 [SMILES: Clc1ccc2c(c1)oc1cccc(Br)c12] |
| 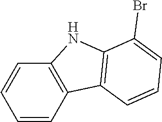 [SMILES: Brc1cccc2c1[nH]c1ccccc12] | 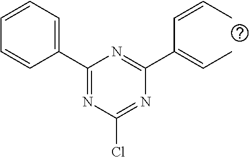 [SMILES: C/C=C\C(=C/C)c1nc(Cl)nc(-c2ccccc2)n1] | 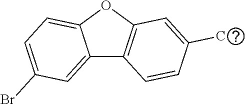 |
| 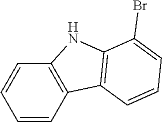 [SMILES: Brc1cccc2c1[nH]c1ccccc12] | 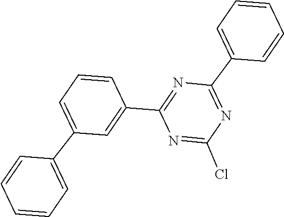 [SMILES: Clc1nc(-c2ccccc2)nc(-c2cccc(-c3ccccc3)c2)n1] | 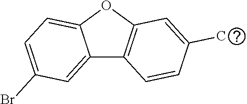 |
| 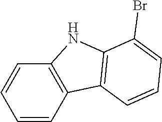 [SMILES: Brc1cccc2c1[nH]c1ccccc12] | 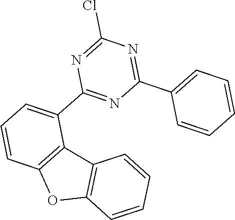 [SMILES: Clc1nc(-c2ccccc2)nc(-c2cccc3oc4ccccc4c23)n1] | 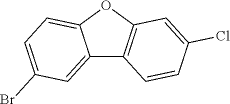 [SMILES: Clc1ccc2c(c1)oc1ccc(Br)cc12] |
| 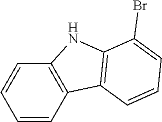 [SMILES: Brc1cccc2c1[nH]c1ccccc12] | 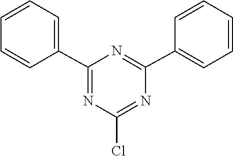 [SMILES: Clc1nc(-c2ccccc2)nc(-c2ccccc2)n1] | 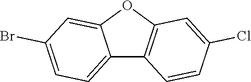 [SMILES: Clc1ccc2c(c1)oc1cc(Br)ccc12] |
| 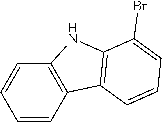 [SMILES: Brc1cccc2c1[nH]c1ccccc12] | 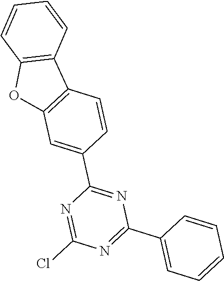 [SMILES: Clc1nc(-c2ccccc2)nc(-c2ccc3c(c2)oc2ccccc23)n1] | 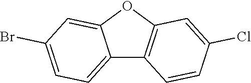 [SMILES: Clc1ccc2c(c1)oc1cc(Br)ccc12] |
| 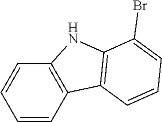 [SMILES: Brc1cccc2c1[nH]c1ccccc12] | 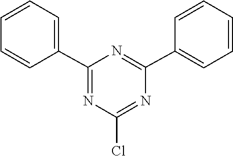 [SMILES: Clc1nc(-c2ccccc2)nc(-c2ccccc2)n1] | 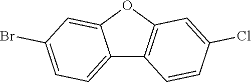 [SMILES: Clc1ccc2c(c1)oc1cc(Br)ccc12] |
| 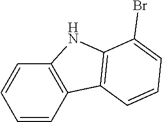 [SMILES: Brc1cccc2c1[nH]c1ccccc12] | 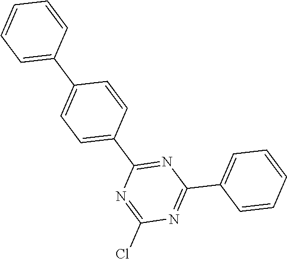 [SMILES: Clc1nc(-c2ccccc2)nc(-c2ccc(-c3ccccc3)cc2)n1] | 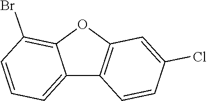 [SMILES: Clc1ccc2c(c1)oc1c(Br)cccc12] |
| 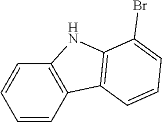 [SMILES: Brc1cccc2c1[nH]c1ccccc12] | 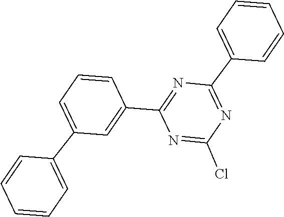 [SMILES: Clc1nc(-c2ccccc2)nc(-c2cccc(-c3ccccc3)c2)n1] | 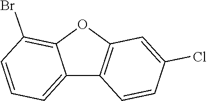 [SMILES: Clc1ccc2c(c1)oc1c(Br)cccc12] |
| 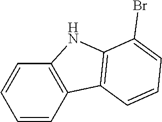 [SMILES: Brc1cccc2c1[nH]c1ccccc12] | 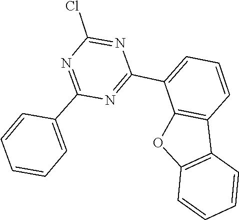 [SMILES: Clc1nc(-c2ccccc2)nc(-c2cccc3c2oc2ccccc23)n1] | 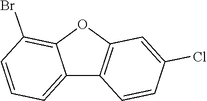 [SMILES: Clc1ccc2c(c1)oc1c(Br)cccc12] |
| 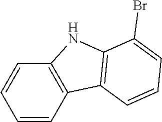 [SMILES: Brc1cccc2c1[nH]c1ccccc12] | 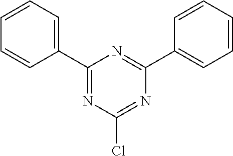 [SMILES: Clc1nc(-c2ccccc2)nc(-c2ccccc2)n1] | 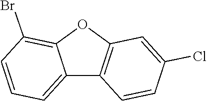 [SMILES: Clc1ccc2c(c1)oc1c(Br)cccc12] |
| 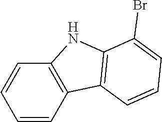 [SMILES: Brc1cccc2c1[nH]c1ccccc12] | 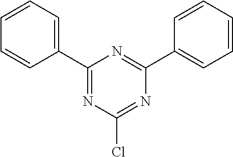 [SMILES: Clc1nc(-c2ccccc2)nc(-c2ccccc2)n1] | 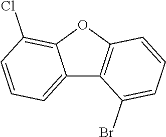 [SMILES: Clc1cccc2c1oc1cccc(Br)c12] |
| 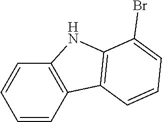 [SMILES: Brc1cccc2c1[nH]c1ccccc12] | 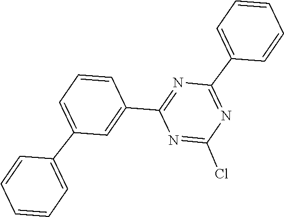 [SMILES: Clc1nc(-c2ccccc2)nc(-c2cccc(-c3ccccc3)c2)n1] | 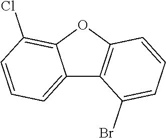 [SMILES: Clc1cccc2c1oc1cccc(Br)c12] |
| 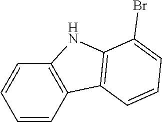 [SMILES: Brc1cccc2c1[nH]c1ccccc12] | 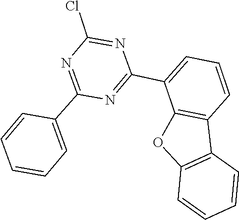 [SMILES: Clc1nc(-c2ccccc2)nc(-c2cccc3c2oc2ccccc23)n1] | 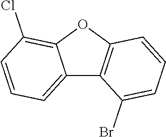 [SMILES: Clc1cccc2c1oc1cccc(Br)c12] |
| 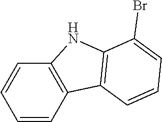 [SMILES: Brc1cccc2c1[nH]c1ccccc12] | 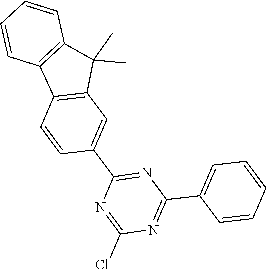 [SMILES: CC1(C)c2ccccc2-c2ccc(-c3nc(Cl)nc(-c4ccccc4)n3)cc21] | 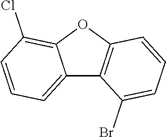 [SMILES: Clc1cccc2c1oc1cccc(Br)c12] |
| 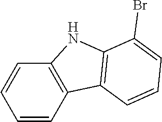 [SMILES: Brc1cccc2c1[nH]c1ccccc12] | 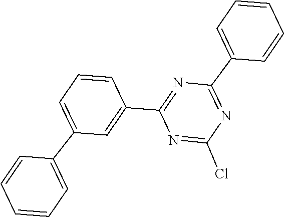 [SMILES: Clc1nc(-c2ccccc2)nc(-c2cccc(-c3ccccc3)c2)n1] | 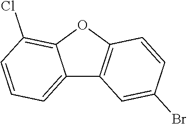 [SMILES: Clc1cccc2c1oc1ccc(Br)cc12] |
| 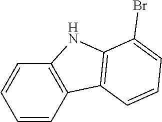 [SMILES: Brc1cccc2c1[nH]c1ccccc12] | 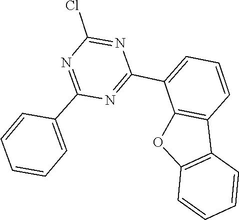 [SMILES: Clc1nc(-c2ccccc2)nc(-c2cccc3c2oc2ccccc23)n1] | 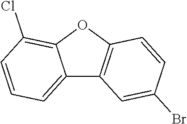 [SMILES: Clc1cccc2c1oc1ccc(Br)cc12] |
| 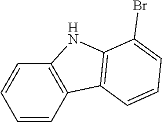 [SMILES: Brc1cccc2c1[nH]c1ccccc12] | 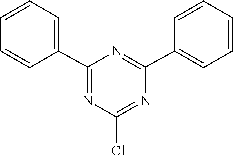 [SMILES: Clc1nc(-c2ccccc2)nc(-c2ccccc2)n1] | 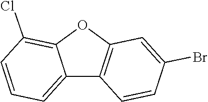 [SMILES: Clc1cccc2c1oc1cc(Br)ccc12] |
| 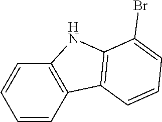 [SMILES: Brc1cccc2c1[nH]c1ccccc12] | 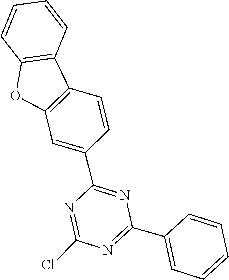 [SMILES: Clc1nc(-c2ccccc2)nc(-c2ccc3c(c2)oc2ccccc23)n1] | 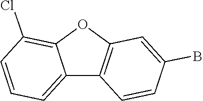 |
| 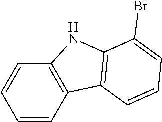 [SMILES: Brc1cccc2c1[nH]c1ccccc12] | 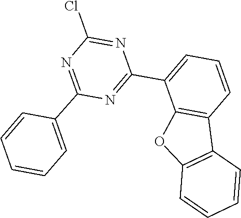 [SMILES: Clc1nc(-c2ccccc2)nc(-c2cccc3c2oc2ccccc23)n1] | 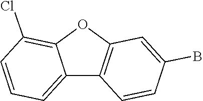 |
| 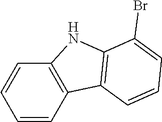 [SMILES: Brc1cccc2c1[nH]c1ccccc12] | 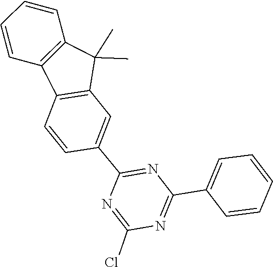 [SMILES: CC1(C)c2ccccc2-c2ccc(-c3nc(Cl)nc(-c4ccccc4)n3)cc21] | 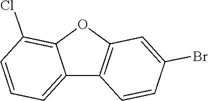 [SMILES: Clc1cccc2c1oc1cc(Br)ccc12] |
| 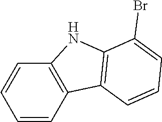 [SMILES: Brc1cccc2c1[nH]c1ccccc12] | 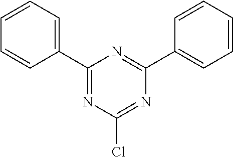 [SMILES: Clc1nc(-c2ccccc2)nc(-c2ccccc2)n1] | 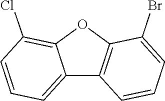 [SMILES: Clc1cccc2c1oc1c(Br)cccc12] |
| 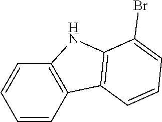 [SMILES: Brc1cccc2c1[nH]c1ccccc12] | 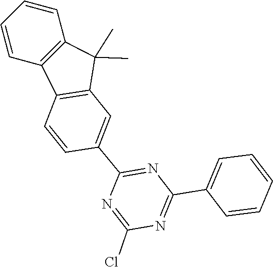 [SMILES: CC1(C)c2ccccc2-c2ccc(-c3nc(Cl)nc(-c4ccccc4)n3)cc21] | 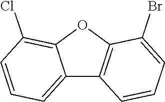 [SMILES: Clc1cccc2c1oc1c(Br)cccc12] |
| 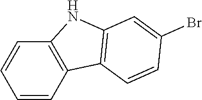 [SMILES: Brc1ccc2c(c1)[nH]c1ccccc12] | 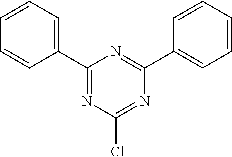 [SMILES: Clc1nc(-c2ccccc2)nc(-c2ccccc2)n1] | 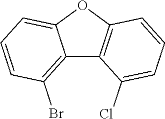 [SMILES: Clc1cccc2oc3cccc(Br)c3c12] |
| 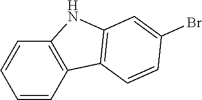 [SMILES: Brc1ccc2c(c1)[nH]c1ccccc12] | 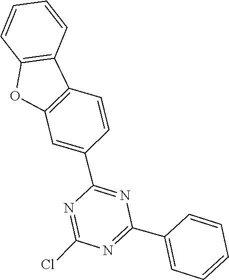 [SMILES: Clc1nc(-c2ccccc2)nc(-c2ccc3c(c2)oc2ccccc23)n1] | 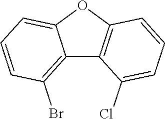 [SMILES: Clc1cccc2oc3cccc(Br)c3c12] |
| 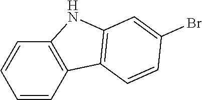 [SMILES: Brc1ccc2c(c1)[nH]c1ccccc12] | 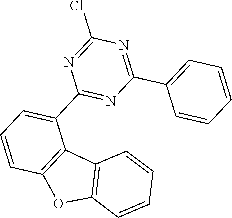 [SMILES: Clc1nc(-c2ccccc2)nc(-c2cccc3oc4ccccc4c23)n1] | 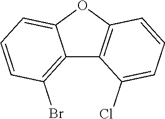 [SMILES: Clc1cccc2oc3cccc(Br)c3c12] |
| 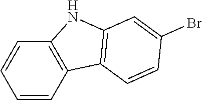 [SMILES: Brc1ccc2c(c1)[nH]c1ccccc12] | 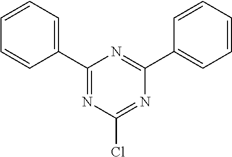 [SMILES: Clc1nc(-c2ccccc2)nc(-c2ccccc2)n1] | 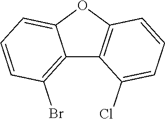 [SMILES: Clc1cccc2oc3cccc(Br)c3c12] |
| 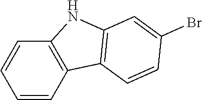 [SMILES: Brc1ccc2c(c1)[nH]c1ccccc12] | 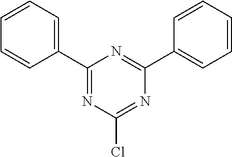 [SMILES: Clc1nc(-c2ccccc2)nc(-c2ccccc2)n1] | 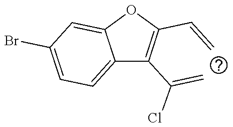 |
| 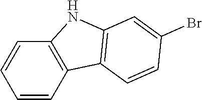 [SMILES: Brc1ccc2c(c1)[nH]c1ccccc12] | 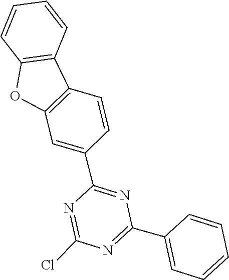 [SMILES: Clc1nc(-c2ccccc2)nc(-c2ccc3c(c2)oc2ccccc23)n1] | 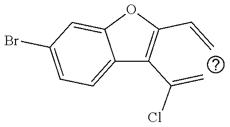 |
| 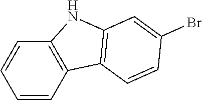 [SMILES: Brc1ccc2c(c1)[nH]c1ccccc12] | 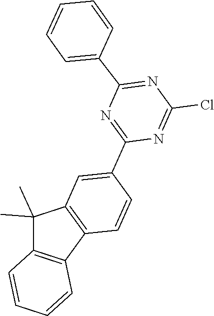 [SMILES: CC1(C)c2ccccc2-c2ccc(-c3nc(Cl)nc(-c4ccccc4)n3)cc21] | 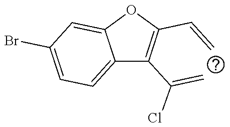 |
| 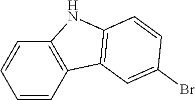 [SMILES: Brc1ccc2[nH]c3ccccc3c2c1] | 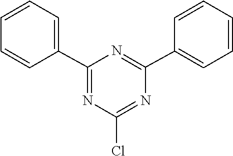 [SMILES: Clc1nc(-c2ccccc2)nc(-c2ccccc2)n1] | 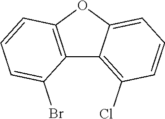 [SMILES: Clc1cccc2oc3cccc(Br)c3c12] |
| 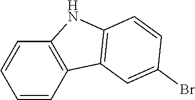 [SMILES: Brc1ccc2[nH]c3ccccc3c2c1] | 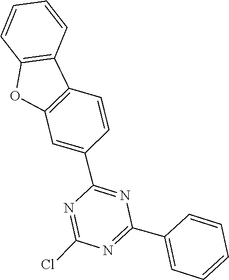 [SMILES: Clc1nc(-c2ccccc2)nc(-c2ccc3c(c2)oc2ccccc23)n1] | 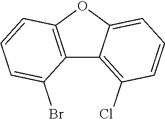 [SMILES: Clc1cccc2oc3cccc(Br)c3c12] |
| 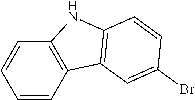 [SMILES: Brc1ccc2[nH]c3ccccc3c2c1] | 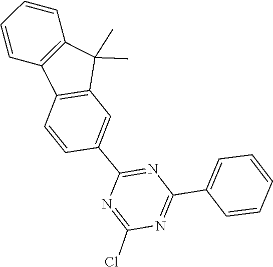 [SMILES: CC1(C)c2ccccc2-c2ccc(-c3nc(Cl)nc(-c4ccccc4)n3)cc21] | 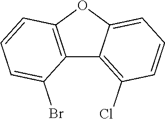 [SMILES: Clc1cccc2oc3cccc(Br)c3c12] |
| 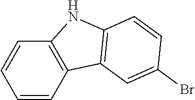 [SMILES: Brc1ccc2[nH]c3ccccc3c2c1] | 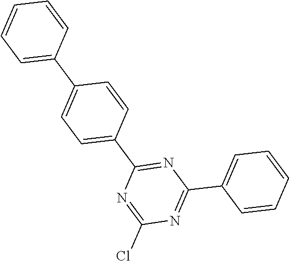 [SMILES: Clc1nc(-c2ccccc2)nc(-c2ccc(-c3ccccc3)cc2)n1] | 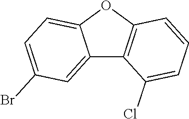 [SMILES: Clc1cccc2oc3ccc(Br)cc3c12] |
| 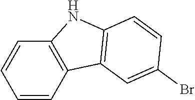 [SMILES: Brc1ccc2[nH]c3ccccc3c2c1] | 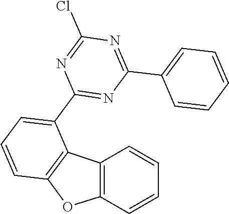 [SMILES: Clc1nc(-c2ccccc2)nc(-c2cccc3oc4ccccc4c23)n1] | 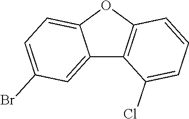 [SMILES: Clc1cccc2oc3ccc(Br)cc3c12] |
| 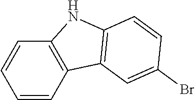 [SMILES: Brc1ccc2[nH]c3ccccc3c2c1] | 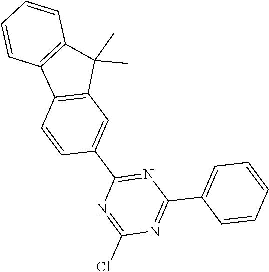 [SMILES: CC1(C)c2ccccc2-c2ccc(-c3nc(Cl)nc(-c4ccccc4)n3)cc21] | 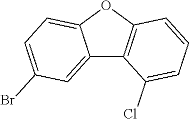 [SMILES: Clc1cccc2oc3ccc(Br)cc3c12] |
| 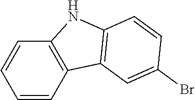 [SMILES: Brc1ccc2[nH]c3ccccc3c2c1] | 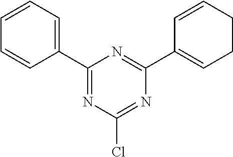 [SMILES: Clc1nc(C2=CCCC=C2)nc(-c2ccccc2)n1] | 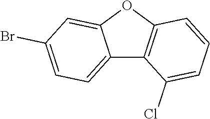 [SMILES: Clc1cccc2oc3cc(Br)ccc3c12] |
| 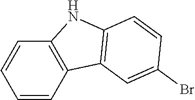 [SMILES: Brc1ccc2[nH]c3ccccc3c2c1] | 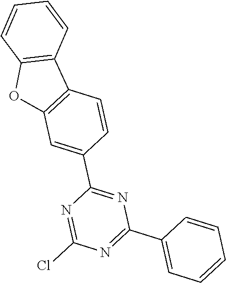 [SMILES: Clc1nc(-c2ccccc2)nc(-c2ccc3c(c2)oc2ccccc23)n1] | 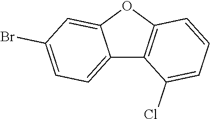 [SMILES: Clc1cccc2oc3cc(Br)ccc3c12] |
| 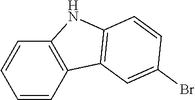 [SMILES: Brc1ccc2[nH]c3ccccc3c2c1] | 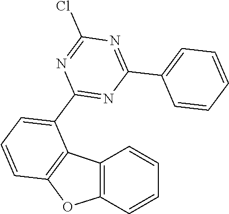 [SMILES: Clc1nc(-c2ccccc2)nc(-c2cccc3oc4ccccc4c23)n1] | 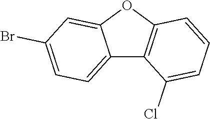 [SMILES: Clc1cccc2oc3cc(Br)ccc3c12] |
| 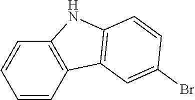 [SMILES: Brc1ccc2[nH]c3ccccc3c2c1] | 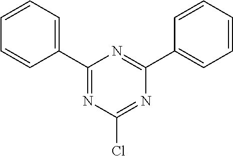 [SMILES: Clc1nc(-c2ccccc2)nc(-c2ccccc2)n1] | 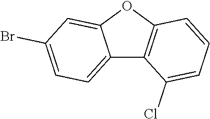 [SMILES: Clc1cccc2oc3cc(Br)ccc3c12] |
| 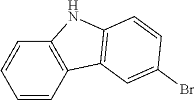 [SMILES: Brc1ccc2[nH]c3ccccc3c2c1] | 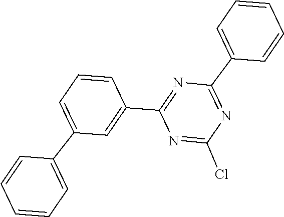 [SMILES: Clc1nc(-c2ccccc2)nc(-c2cccc(-c3ccccc3)c2)n1] | 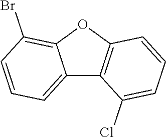 [SMILES: Clc1cccc2oc3c(Br)cccc3c12] |
| 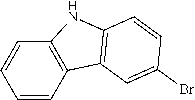 [SMILES: Brc1ccc2[nH]c3ccccc3c2c1] | 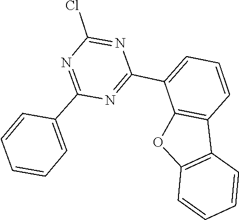 [SMILES: Clc1nc(-c2ccccc2)nc(-c2cccc3c2oc2ccccc23)n1] | 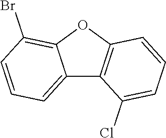 [SMILES: Clc1cccc2oc3c(Br)cccc3c12] |
| 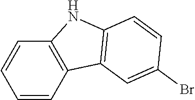 [SMILES: Brc1ccc2[nH]c3ccccc3c2c1] | 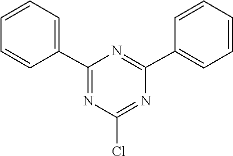 [SMILES: Clc1nc(-c2ccccc2)nc(-c2ccccc2)n1] | 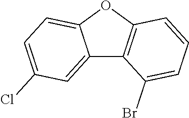 [SMILES: Clc1ccc2oc3cccc(Br)c3c2c1] |
| 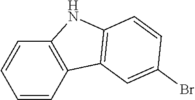 [SMILES: Brc1ccc2[nH]c3ccccc3c2c1] | 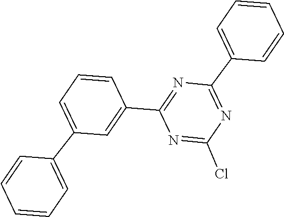 [SMILES: Clc1nc(-c2ccccc2)nc(-c2cccc(-c3ccccc3)c2)n1] | 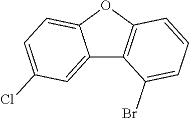 [SMILES: Clc1ccc2oc3cccc(Br)c3c2c1] |
| 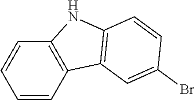 [SMILES: Brc1ccc2[nH]c3ccccc3c2c1] | 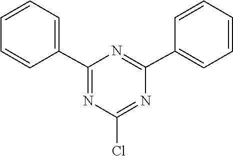 [SMILES: Clc1nc(-c2ccccc2)nc(-c2ccccc2)n1] | 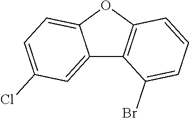 [SMILES: Clc1ccc2oc3cccc(Br)c3c2c1] |
| 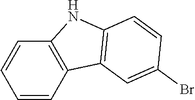 [SMILES: Brc1ccc2[nH]c3ccccc3c2c1] | 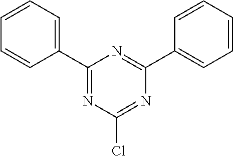 [SMILES: Clc1nc(-c2ccccc2)nc(-c2ccccc2)n1] | 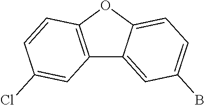 |
| 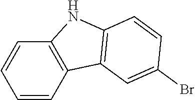 [SMILES: Brc1ccc2[nH]c3ccccc3c2c1] | 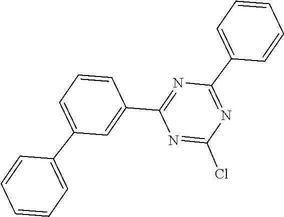 [SMILES: Clc1nc(-c2ccccc2)nc(-c2cccc(-c3ccccc3)c2)n1] | 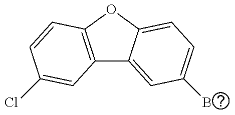 |
| 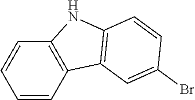 [SMILES: Brc1ccc2[nH]c3ccccc3c2c1] | 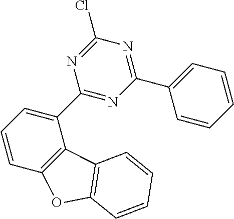 [SMILES: Clc1nc(-c2ccccc2)nc(-c2cccc3oc4ccccc4c23)n1] | 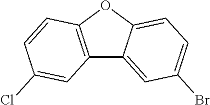 [SMILES: Clc1ccc2oc3ccc(Br)cc3c2c1] |
| 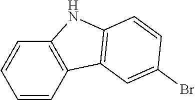 [SMILES: Brc1ccc2[nH]c3ccccc3c2c1] | 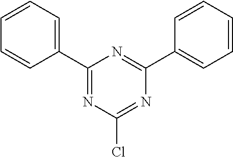 [SMILES: Clc1nc(-c2ccccc2)nc(-c2ccccc2)n1] | 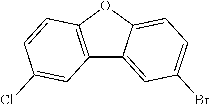 [SMILES: Clc1ccc2oc3ccc(Br)cc3c2c1] |
| 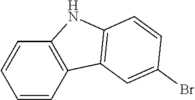 [SMILES: Brc1ccc2[nH]c3ccccc3c2c1] | 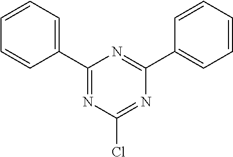 [SMILES: Clc1nc(-c2ccccc2)nc(-c2ccccc2)n1] | 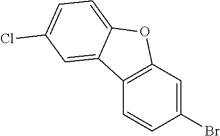 [SMILES: Clc1ccc2oc3cc(Br)ccc3c2c1] |
| 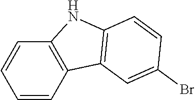 [SMILES: Brc1ccc2[nH]c3ccccc3c2c1] | 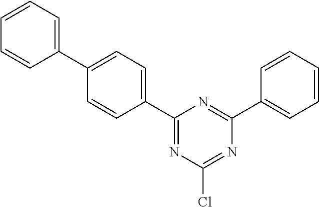 [SMILES: Clc1nc(-c2ccccc2)nc(-c2ccc(-c3ccccc3)cc2)n1] | 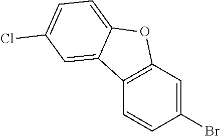 [SMILES: Clc1ccc2oc3cc(Br)ccc3c2c1] |
| 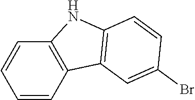 [SMILES: Brc1ccc2[nH]c3ccccc3c2c1] | 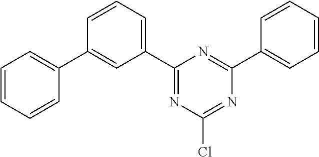 [SMILES: Clc1nc(-c2ccccc2)nc(-c2cccc(-c3ccccc3)c2)n1] | 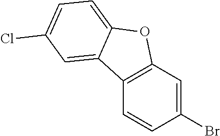 [SMILES: Clc1ccc2oc3cc(Br)ccc3c2c1] |
| 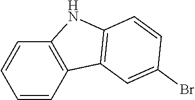 [SMILES: Brc1ccc2[nH]c3ccccc3c2c1] | 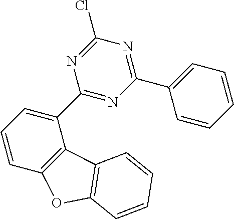 [SMILES: Clc1nc(-c2ccccc2)nc(-c2cccc3oc4ccccc4c23)n1] | 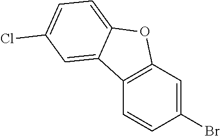 [SMILES: Clc1ccc2oc3cc(Br)ccc3c2c1] |
| 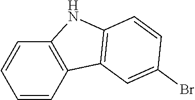 [SMILES: Brc1ccc2[nH]c3ccccc3c2c1] | 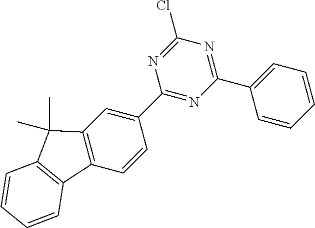 [SMILES: CC1(C)c2ccccc2-c2ccc(-c3nc(Cl)nc(-c4ccccc4)n3)cc21] | 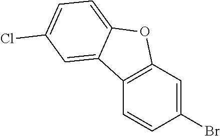 [SMILES: Clc1ccc2oc3cc(Br)ccc3c2c1] |
| 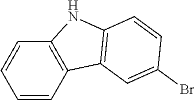 [SMILES: Brc1ccc2[nH]c3ccccc3c2c1] | 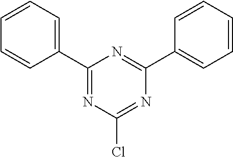 [SMILES: Clc1nc(-c2ccccc2)nc(-c2ccccc2)n1] | 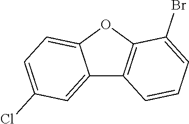 [SMILES: Clc1ccc2oc3c(Br)cccc3c2c1] |
| 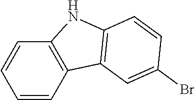 [SMILES: Brc1ccc2[nH]c3ccccc3c2c1] | 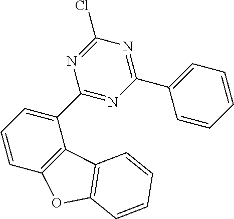 [SMILES: Clc1nc(-c2ccccc2)nc(-c2cccc3oc4ccccc4c23)n1] | 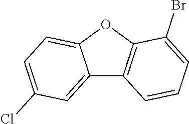 [SMILES: Clc1ccc2oc3c(Br)cccc3c2c1] |
| 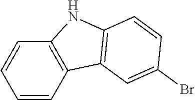 [SMILES: Brc1ccc2[nH]c3ccccc3c2c1] | 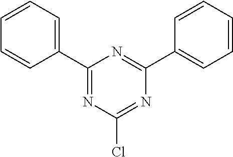 [SMILES: Clc1nc(-c2ccccc2)nc(-c2ccccc2)n1] | 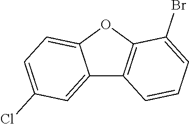 [SMILES: Clc1ccc2oc3c(Br)cccc3c2c1] |
| 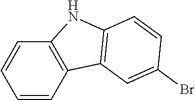 [SMILES: Brc1ccc2[nH]c3ccccc3c2c1] | 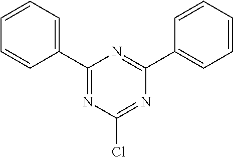 [SMILES: Clc1nc(-c2ccccc2)nc(-c2ccccc2)n1] | 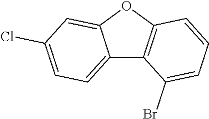 [SMILES: Clc1ccc2c(c1)oc1cccc(Br)c12] |
| 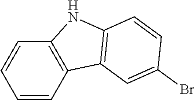 [SMILES: Brc1ccc2[nH]c3ccccc3c2c1] | 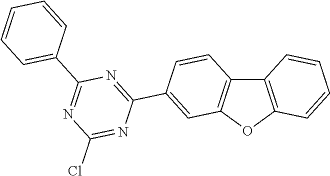 [SMILES: Clc1nc(-c2ccccc2)nc(-c2ccc3c(c2)oc2ccccc23)n1] | 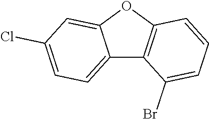 [SMILES: Clc1ccc2c(c1)oc1cccc(Br)c12] |
| 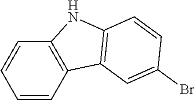 [SMILES: Brc1ccc2[nH]c3ccccc3c2c1] | 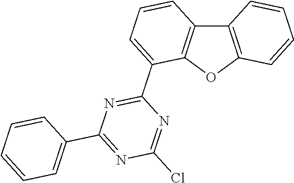 [SMILES: Clc1nc(-c2ccccc2)nc(-c2cccc3c2oc2ccccc23)n1] | 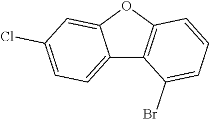 [SMILES: Clc1ccc2c(c1)oc1cccc(Br)c12] |
| 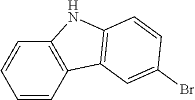 [SMILES: Brc1ccc2[nH]c3ccccc3c2c1] | 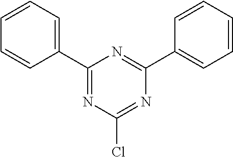 [SMILES: Clc1nc(-c2ccccc2)nc(-c2ccccc2)n1] | 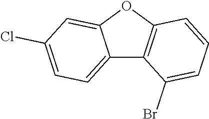 [SMILES: Clc1ccc2c(c1)oc1cccc(Br)c12] |
| 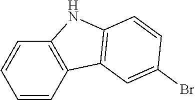 [SMILES: Brc1ccc2[nH]c3ccccc3c2c1] | 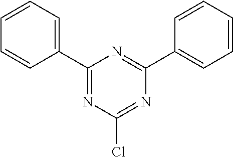 [SMILES: Clc1nc(-c2ccccc2)nc(-c2ccccc2)n1] | 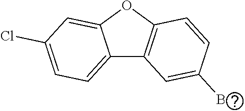 |
| 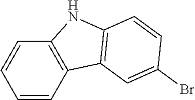 [SMILES: Brc1ccc2[nH]c3ccccc3c2c1] | 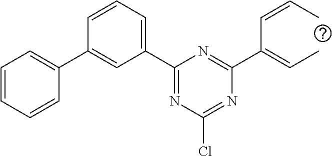 [SMILES: C/C=C\C(=C/C)c1nc(Cl)nc(-c2cccc(-c3ccccc3)c2)n1] | 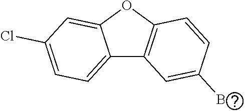 |
| 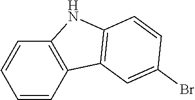 [SMILES: Brc1ccc2[nH]c3ccccc3c2c1] | 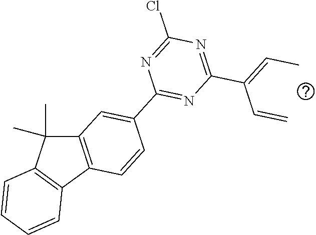 [SMILES: C=C/C(=C\C)c1nc(Cl)nc(-c2ccc3c(c2)C(C)(C)c2ccccc2-3)n1] | 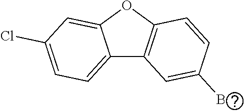 |
| 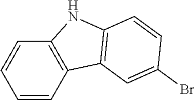 [SMILES: Brc1ccc2[nH]c3ccccc3c2c1] | 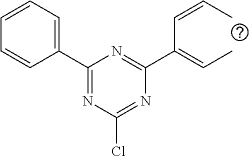 [SMILES: C/C=C\C(=C/C)c1nc(Cl)nc(-c2ccccc2)n1] | 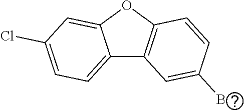 |
| 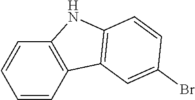 [SMILES: Brc1ccc2[nH]c3ccccc3c2c1] | 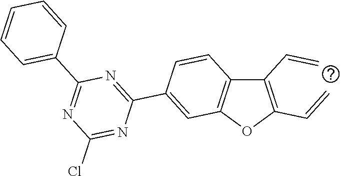 [SMILES: C=Cc1oc2cc(-c3nc(Cl)nc(-c4ccccc4)n3)ccc2c1C=C] | 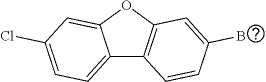 |
| 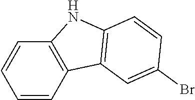 [SMILES: Brc1ccc2[nH]c3ccccc3c2c1] | 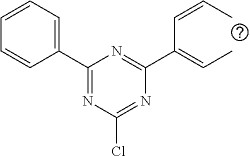 [SMILES: C/C=C\C(=C/C)c1nc(Cl)nc(-c2ccccc2)n1] | 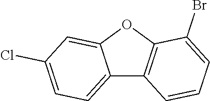 [SMILES: Clc1ccc2c(c1)oc1c(Br)cccc12] |
| 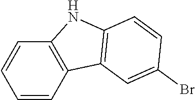 [SMILES: Brc1ccc2[nH]c3ccccc3c2c1] | 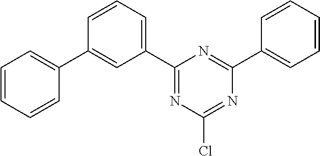 [SMILES: Clc1nc(-c2ccccc2)nc(-c2cccc(-c3ccccc3)c2)n1] | 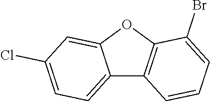 [SMILES: Clc1ccc2c(c1)oc1c(Br)cccc12] |
| 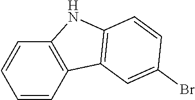 [SMILES: Brc1ccc2[nH]c3ccccc3c2c1] | 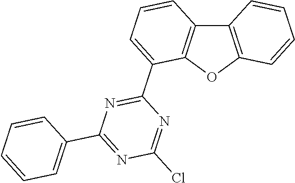 [SMILES: Clc1nc(-c2ccccc2)nc(-c2cccc3c2oc2ccccc23)n1] | 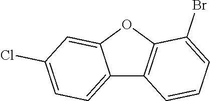 [SMILES: Clc1ccc2c(c1)oc1c(Br)cccc12] |
| 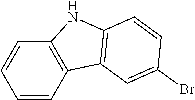 [SMILES: Brc1ccc2[nH]c3ccccc3c2c1] | 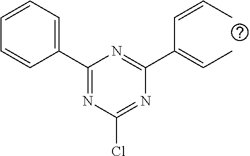 [SMILES: C/C=C\C(=C/C)c1nc(Cl)nc(-c2ccccc2)n1] | 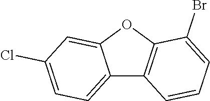 [SMILES: Clc1ccc2c(c1)oc1c(Br)cccc12] |
| 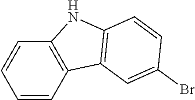 [SMILES: Brc1ccc2[nH]c3ccccc3c2c1] | 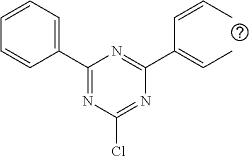 [SMILES: C/C=C\C(=C/C)c1nc(Cl)nc(-c2ccccc2)n1] | 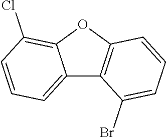 [SMILES: Clc1cccc2c1oc1cccc(Br)c12] |
| 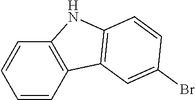 [SMILES: Brc1ccc2[nH]c3ccccc3c2c1] | 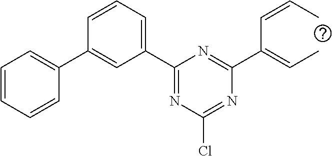 [SMILES: C/C=C\C(=C/C)c1nc(Cl)nc(-c2cccc(-c3ccccc3)c2)n1] | 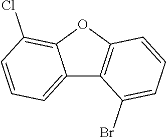 [SMILES: Clc1cccc2c1oc1cccc(Br)c12] |
| 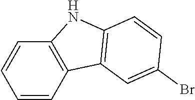 [SMILES: Brc1ccc2[nH]c3ccccc3c2c1] | 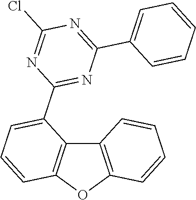 [SMILES: Clc1nc(-c2ccccc2)nc(-c2cccc3oc4ccccc4c23)n1] | 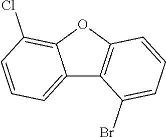 [SMILES: Clc1cccc2c1oc1cccc(Br)c12] |
| 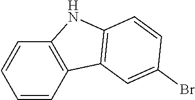 [SMILES: Brc1ccc2[nH]c3ccccc3c2c1] | 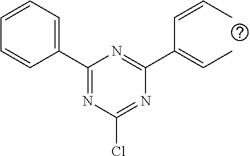 [SMILES: C/C=C\C(=C/C)c1nc(Cl)nc(-c2ccccc2)n1] | 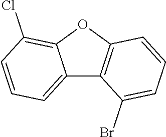 [SMILES: Clc1cccc2c1oc1cccc(Br)c12] |
| 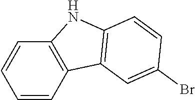 [SMILES: Brc1ccc2[nH]c3ccccc3c2c1] | 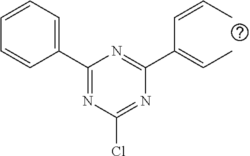 [SMILES: C/C=C\C(=C/C)c1nc(Cl)nc(-c2ccccc2)n1] | 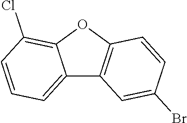 [SMILES: Clc1cccc2c1oc1ccc(Br)cc12] |
| 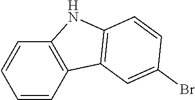 [SMILES: Brc1ccc2[nH]c3ccccc3c2c1] | 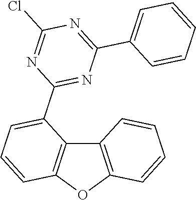 [SMILES: Clc1nc(-c2ccccc2)nc(-c2cccc3oc4ccccc4c23)n1] | 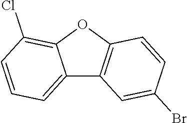 [SMILES: Clc1cccc2c1oc1ccc(Br)cc12] |
| 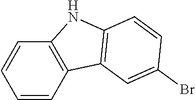 [SMILES: Brc1ccc2[nH]c3ccccc3c2c1] | 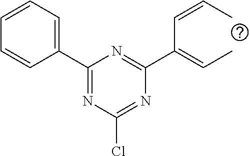 [SMILES: C/C=C\C(=C/C)c1nc(Cl)nc(-c2ccccc2)n1] | 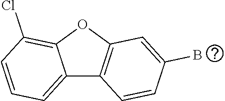 |
| 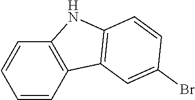 [SMILES: Brc1ccc2[nH]c3ccccc3c2c1] | 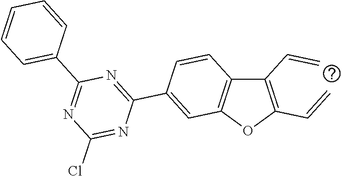 [SMILES: C=Cc1oc2cc(-c3nc(Cl)nc(-c4ccccc4)n3)ccc2c1C=C] | 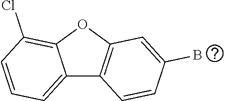 |
| 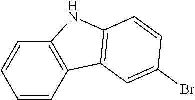 [SMILES: Brc1ccc2[nH]c3ccccc3c2c1] | 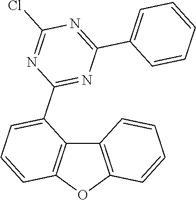 [SMILES: Clc1nc(-c2ccccc2)nc(-c2cccc3oc4ccccc4c23)n1] | 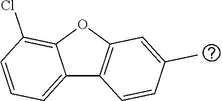 |
| 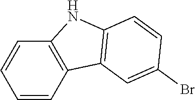 [SMILES: Brc1ccc2[nH]c3ccccc3c2c1] | 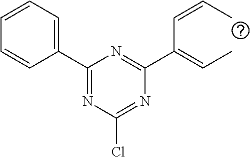 [SMILES: C/C=C\C(=C/C)c1nc(Cl)nc(-c2ccccc2)n1] | 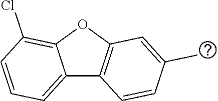 |
| 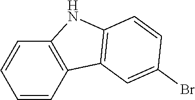 [SMILES: Brc1ccc2[nH]c3ccccc3c2c1] | 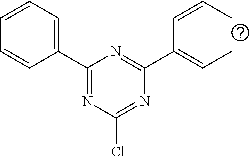 [SMILES: C/C=C\C(=C/C)c1nc(Cl)nc(-c2ccccc2)n1] | 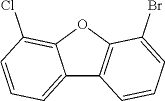 [SMILES: Clc1cccc2c1oc1c(Br)cccc12] |
| 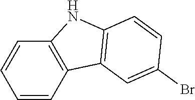 [SMILES: Brc1ccc2[nH]c3ccccc3c2c1] | 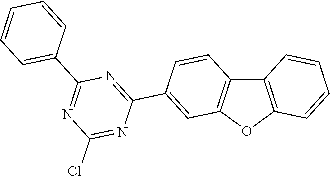 [SMILES: Clc1nc(-c2ccccc2)nc(-c2ccc3c(c2)oc2ccccc23)n1] | 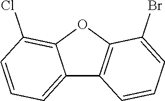 [SMILES: Clc1cccc2c1oc1c(Br)cccc12] |
| 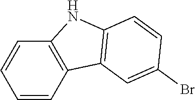 [SMILES: Brc1ccc2[nH]c3ccccc3c2c1] | 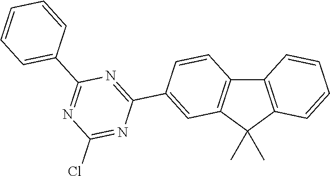 [SMILES: CC1(C)c2ccccc2-c2ccc(-c3nc(Cl)nc(-c4ccccc4)n3)cc21] | 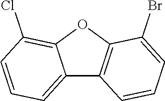 [SMILES: Clc1cccc2c1oc1c(Br)cccc12] |
| 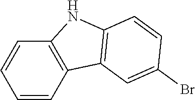 [SMILES: Brc1ccc2[nH]c3ccccc3c2c1] | 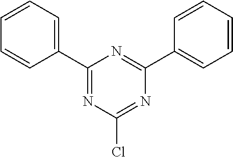 [SMILES: Clc1nc(-c2ccccc2)nc(-c2ccccc2)n1] | 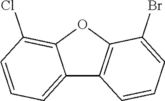 [SMILES: Clc1cccc2c1oc1c(Br)cccc12] |
| 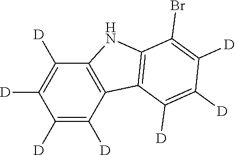 [SMILES: [2H]c1c([2H])c([2H])c2c([nH]c3c(Br)c([2H])c([2H])c([2H])c32)c1[2H]] | 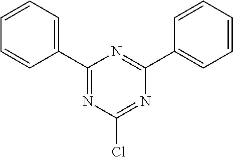 [SMILES: Clc1nc(-c2ccccc2)nc(-c2ccccc2)n1] | 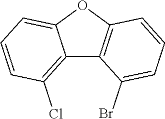 [SMILES: Clc1cccc2oc3cccc(Br)c3c12] |
| 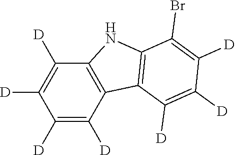 [SMILES: [2H]c1c([2H])c([2H])c2c([nH]c3c(Br)c([2H])c([2H])c([2H])c32)c1[2H]] | 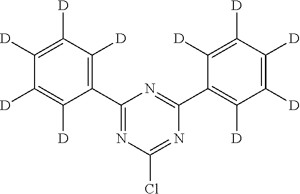 [SMILES: [2H]c1c([2H])c([2H])c(-c2nc(Cl)nc(-c3c([2H])c([2H])c([2H])c([2H])c3[2H])n2)c([2H])c1[2H]] | 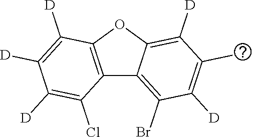 [SMILES: [2H]c1c([2H])c(Cl)c2c(oc3c([2H])c(C)c([2H])c(Br)c32)c1[2H]] |
| 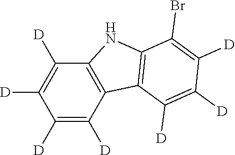 [SMILES: [2H]c1c([2H])c([2H])c2c([nH]c3c(Br)c([2H])c([2H])c([2H])c32)c1[2H]] | 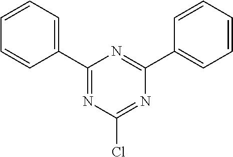 [SMILES: Clc1nc(-c2ccccc2)nc(-c2ccccc2)n1] | 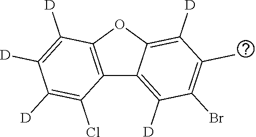 [SMILES: [2H]c1c([2H])c(Cl)c2c(oc3c([2H])c(C)c(Br)c([2H])c32)c1[2H]] |
| 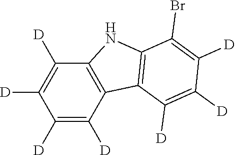 [SMILES: [2H]c1c([2H])c([2H])c2c([nH]c3c(Br)c([2H])c([2H])c([2H])c32)c1[2H]] | 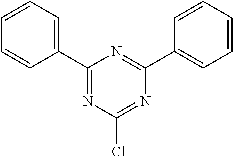 [SMILES: Clc1nc(-c2ccccc2)nc(-c2ccccc2)n1] | 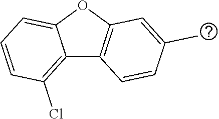 |
| 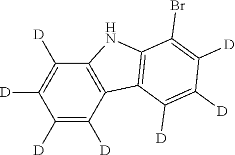 [SMILES: [2H]c1c([2H])c([2H])c2c([nH]c3c(Br)c([2H])c([2H])c([2H])c32)c1[2H]] | 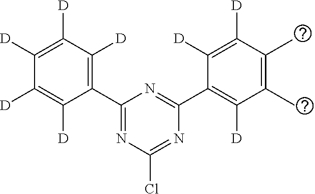 [SMILES: [2H]c1c([2H])c([2H])c(-c2nc(Cl)nc(-c3c([2H])c([2H])c(C)c(C)c3[2H])n2)c([2H])c1[2H]] | 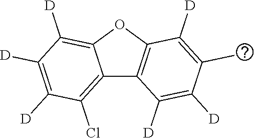 [SMILES: [2H]c1c([2H])c(Cl)c2c(oc3c([2H])c(C)c([2H])c([2H])c32)c1[2H]] |
| 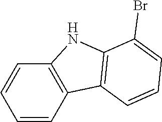 [SMILES: Brc1cccc2c1[nH]c1ccccc12] | 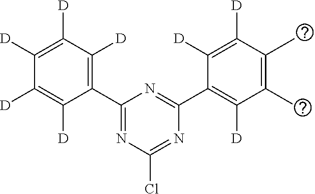 [SMILES: [2H]c1c([2H])c([2H])c(-c2nc(Cl)nc(-c3c([2H])c([2H])c(C)c(C)c3[2H])n2)c([2H])c1[2H]] | 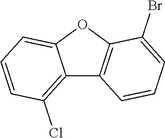 [SMILES: Clc1cccc2oc3c(Br)cccc3c12] |
| 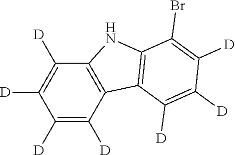 [SMILES: [2H]c1c([2H])c([2H])c2c([nH]c3c(Br)c([2H])c([2H])c([2H])c32)c1[2H]] | 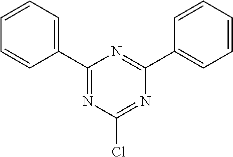 [SMILES: Clc1nc(-c2ccccc2)nc(-c2ccccc2)n1] | 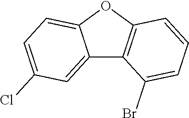 [SMILES: Clc1ccc2oc3cccc(Br)c3c2c1] |
| 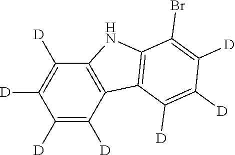 [SMILES: [2H]c1c([2H])c([2H])c2c([nH]c3c(Br)c([2H])c([2H])c([2H])c32)c1[2H]] | 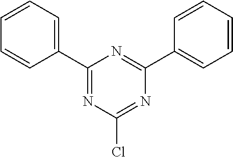 [SMILES: Clc1nc(-c2ccccc2)nc(-c2ccccc2)n1] | 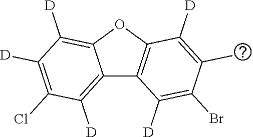 [SMILES: [2H]c1c(Cl)c([2H])c2c(oc3c([2H])c(C)c(Br)c([2H])c32)c1[2H]] |
| 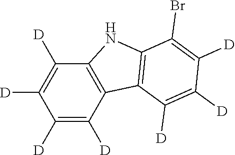 [SMILES: [2H]c1c([2H])c([2H])c2c([nH]c3c(Br)c([2H])c([2H])c([2H])c32)c1[2H]] | 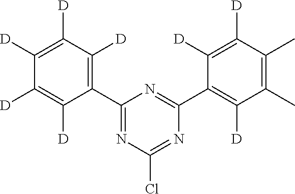 [SMILES: [2H]c1c([2H])c([2H])c(-c2nc(Cl)nc(-c3c([2H])c([2H])c(C)c(C)c3[2H])n2)c([2H])c1[2H]] | 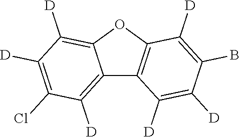 |
| 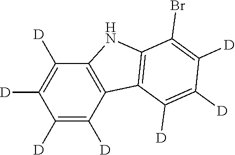 [SMILES: [2H]c1c([2H])c([2H])c2c([nH]c3c(Br)c([2H])c([2H])c([2H])c32)c1[2H]] | 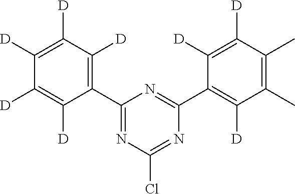 [SMILES: [2H]c1c([2H])c([2H])c(-c2nc(Cl)nc(-c3c([2H])c([2H])c(C)c(C)c3[2H])n2)c([2H])c1[2H]] | 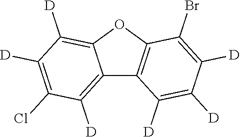 [SMILES: [2H]c1c(Cl)c([2H])c2c(oc3c(Br)c([2H])c([2H])c([2H])c32)c1[2H]] |
| 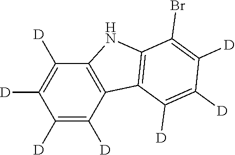 [SMILES: [2H]c1c([2H])c([2H])c2c([nH]c3c(Br)c([2H])c([2H])c([2H])c32)c1[2H]] | 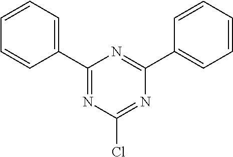 [SMILES: Clc1nc(-c2ccccc2)nc(-c2ccccc2)n1] | 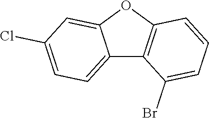 [SMILES: Clc1ccc2c(c1)oc1cccc(Br)c12] |
| 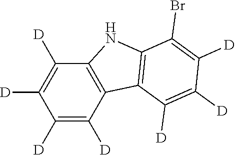 [SMILES: [2H]c1c([2H])c([2H])c2c([nH]c3c(Br)c([2H])c([2H])c([2H])c32)c1[2H]] | 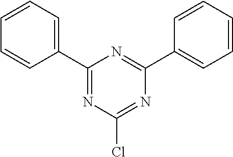 [SMILES: Clc1nc(-c2ccccc2)nc(-c2ccccc2)n1] | 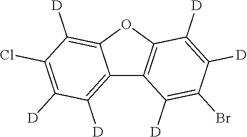 [SMILES: [2H]c1c(Br)c([2H])c2c(oc3c([2H])c(Cl)c([2H])c([2H])c32)c1[2H]] |
| 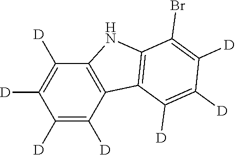 [SMILES: [2H]c1c([2H])c([2H])c2c([nH]c3c(Br)c([2H])c([2H])c([2H])c32)c1[2H]] | 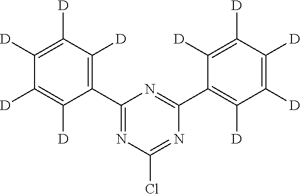 [SMILES: [2H]c1c([2H])c([2H])c(-c2nc(Cl)nc(-c3c([2H])c([2H])c([2H])c([2H])c3[2H])n2)c([2H])c1[2H]] | 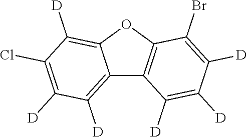 [SMILES: [2H]c1c([2H])c([2H])c2c(oc3c([2H])c(Cl)c([2H])c([2H])c32)c1Br] |
| 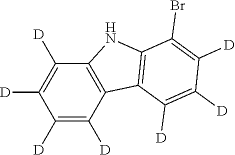 [SMILES: [2H]c1c([2H])c([2H])c2c([nH]c3c(Br)c([2H])c([2H])c([2H])c32)c1[2H]] | 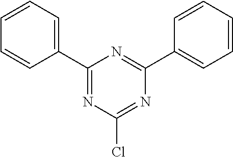 [SMILES: Clc1nc(-c2ccccc2)nc(-c2ccccc2)n1] | 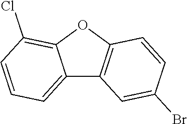 [SMILES: Clc1cccc2c1oc1ccc(Br)cc12] |
| 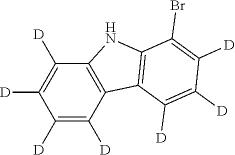 [SMILES: [2H]c1c([2H])c([2H])c2c([nH]c3c(Br)c([2H])c([2H])c([2H])c32)c1[2H]] | 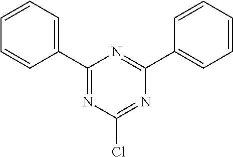 [SMILES: Clc1nc(-c2ccccc2)nc(-c2ccccc2)n1] | 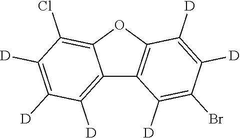 [SMILES: [2H]c1c(Br)c([2H])c2c(oc3c(Cl)c([2H])c([2H])c([2H])c32)c1[2H]] |
| 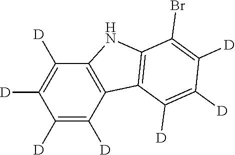 [SMILES: [2H]c1c([2H])c([2H])c2c([nH]c3c(Br)c([2H])c([2H])c([2H])c32)c1[2H]] | 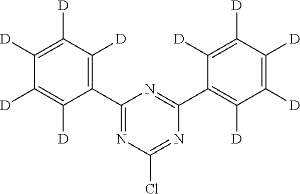 [SMILES: [2H]c1c([2H])c([2H])c(-c2nc(Cl)nc(-c3c([2H])c([2H])c([2H])c([2H])c3[2H])n2)c([2H])c1[2H]] | 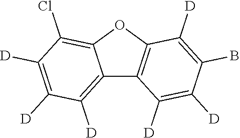 |
| 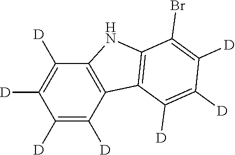 [SMILES: [2H]c1c([2H])c([2H])c2c([nH]c3c(Br)c([2H])c([2H])c([2H])c32)c1[2H]] | 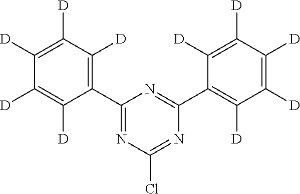 [SMILES: [2H]c1c([2H])c([2H])c(-c2nc(Cl)nc(-c3c([2H])c([2H])c([2H])c([2H])c3[2H])n2)c([2H])c1[2H]] | 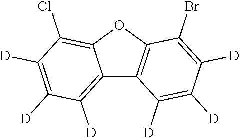 [SMILES: [2H]c1c([2H])c([2H])c2c(oc3c(Br)c([2H])c([2H])c([2H])c32)c1Cl] |
| 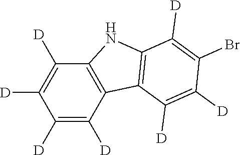 [SMILES: [2H]c1c([2H])c([2H])c2c([nH]c3c([2H])c(Br)c([2H])c([2H])c32)c1[2H]] | 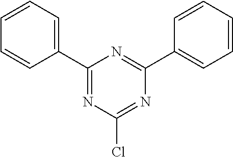 [SMILES: Clc1nc(-c2ccccc2)nc(-c2ccccc2)n1] | 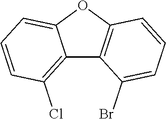 [SMILES: Clc1cccc2oc3cccc(Br)c3c12] |
| 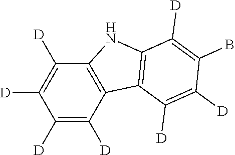 | 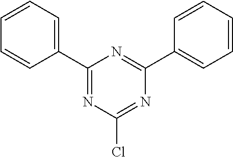 [SMILES: Clc1nc(-c2ccccc2)nc(-c2ccccc2)n1] | 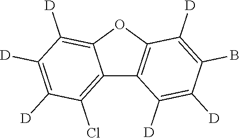 |
| 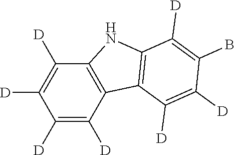 | 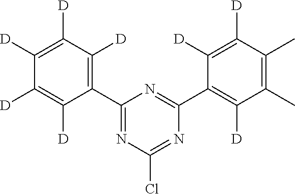 [SMILES: [2H]c1c([2H])c([2H])c(-c2nc(Cl)nc(-c3c([2H])c([2H])c(C)c(C)c3[2H])n2)c([2H])c1[2H]] | 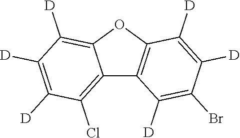 [SMILES: [2H]c1c(Br)c([2H])c2c(oc3c([2H])c([2H])c([2H])c(Cl)c32)c1[2H]] |
| 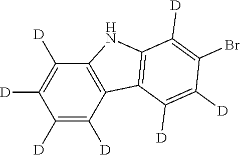 [SMILES: [2H]c1c([2H])c([2H])c2c([nH]c3c([2H])c(Br)c([2H])c([2H])c32)c1[2H]] | 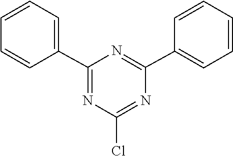 [SMILES: Clc1nc(-c2ccccc2)nc(-c2ccccc2)n1] | 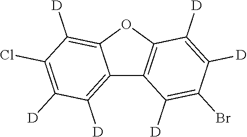 [SMILES: [2H]c1c(Br)c([2H])c2c(oc3c([2H])c(Cl)c([2H])c([2H])c32)c1[2H]] |
| 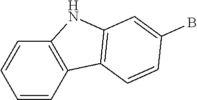 | 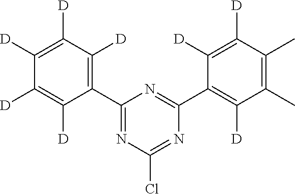 [SMILES: [2H]c1c([2H])c([2H])c(-c2nc(Cl)nc(-c3c([2H])c([2H])c(C)c(C)c3[2H])n2)c([2H])c1[2H]] | 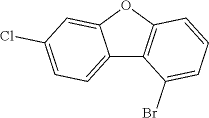 [SMILES: Clc1ccc2c(c1)oc1cccc(Br)c12] |
| 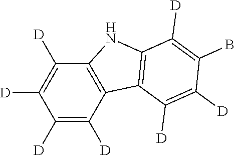 | 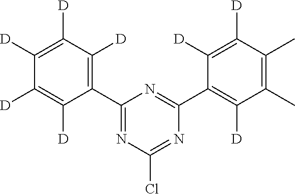 [SMILES: [2H]c1c([2H])c([2H])c(-c2nc(Cl)nc(-c3c([2H])c([2H])c(C)c(C)c3[2H])n2)c([2H])c1[2H]] | 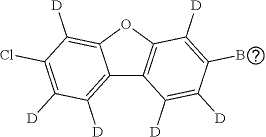 |
| 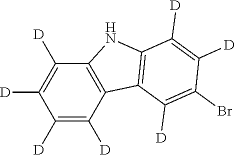 [SMILES: [2H]c1c([2H])c([2H])c2c([nH]c3c([2H])c([2H])c(Br)c([2H])c32)c1[2H]] | 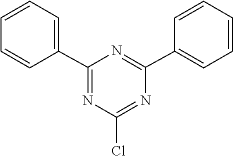 [SMILES: Clc1nc(-c2ccccc2)nc(-c2ccccc2)n1] | 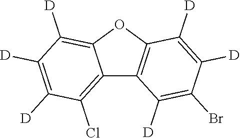 [SMILES: [2H]c1c(Br)c([2H])c2c(oc3c([2H])c([2H])c([2H])c(Cl)c32)c1[2H]] |
| 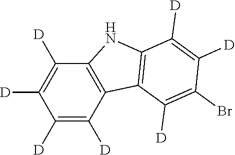 [SMILES: [2H]c1c([2H])c([2H])c2c([nH]c3c([2H])c([2H])c(Br)c([2H])c32)c1[2H]] | 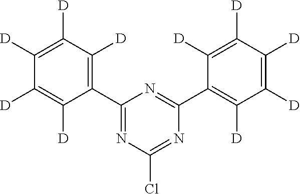 [SMILES: [2H]c1c([2H])c([2H])c(-c2nc(Cl)nc(-c3c([2H])c([2H])c([2H])c([2H])c3[2H])n2)c([2H])c1[2H]] | 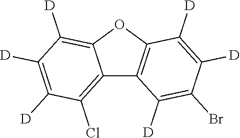 [SMILES: [2H]c1c(Br)c([2H])c2c(oc3c([2H])c([2H])c([2H])c(Cl)c32)c1[2H]] |
| 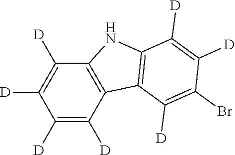 [SMILES: [2H]c1c([2H])c([2H])c2c([nH]c3c([2H])c([2H])c(Br)c([2H])c32)c1[2H]] | 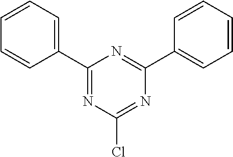 [SMILES: Clc1nc(-c2ccccc2)nc(-c2ccccc2)n1] | 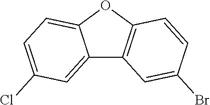 [SMILES: Clc1ccc2oc3ccc(Br)cc3c2c1] |
| 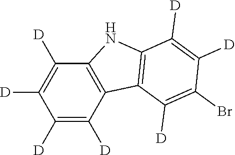 [SMILES: [2H]c1c([2H])c([2H])c2c([nH]c3c([2H])c([2H])c(Br)c([2H])c32)c1[2H]] | 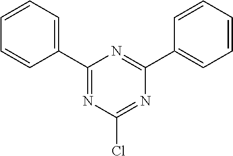 [SMILES: Clc1nc(-c2ccccc2)nc(-c2ccccc2)n1] | 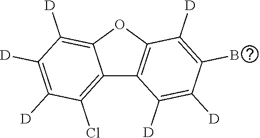 |
| 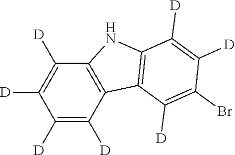 [SMILES: [2H]c1c([2H])c([2H])c2c([nH]c3c([2H])c([2H])c(Br)c([2H])c32)c1[2H]] | 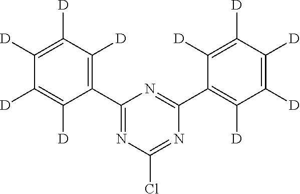 [SMILES: [2H]c1c([2H])c([2H])c(-c2nc(Cl)nc(-c3c([2H])c([2H])c([2H])c([2H])c3[2H])n2)c([2H])c1[2H]] | 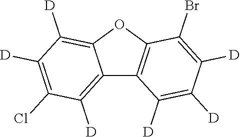 [SMILES: [2H]c1c(Cl)c([2H])c2c(oc3c(Br)c([2H])c([2H])c([2H])c32)c1[2H]] |
| 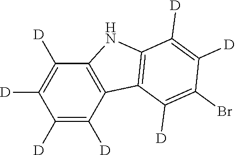 [SMILES: [2H]c1c([2H])c([2H])c2c([nH]c3c([2H])c([2H])c(Br)c([2H])c32)c1[2H]] | 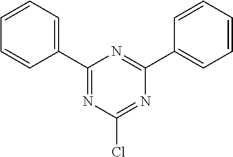 [SMILES: Clc1nc(-c2ccccc2)nc(-c2ccccc2)n1] | 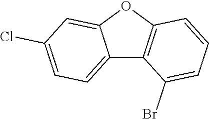 [SMILES: Clc1ccc2c(c1)oc1cccc(Br)c12] |
| 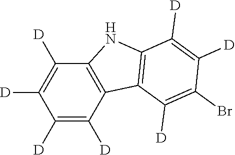 [SMILES: [2H]c1c([2H])c([2H])c2c([nH]c3c([2H])c([2H])c(Br)c([2H])c32)c1[2H]] | 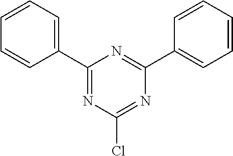 [SMILES: Clc1nc(-c2ccccc2)nc(-c2ccccc2)n1] | 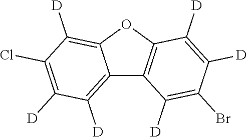 [SMILES: [2H]c1c(Br)c([2H])c2c(oc3c([2H])c(Cl)c([2H])c([2H])c32)c1[2H]] |
| 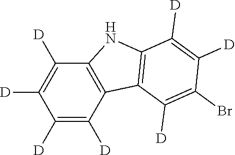 [SMILES: [2H]c1c([2H])c([2H])c2c([nH]c3c([2H])c([2H])c(Br)c([2H])c32)c1[2H]] | 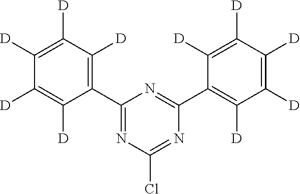 [SMILES: [2H]c1c([2H])c([2H])c(-c2nc(Cl)nc(-c3c([2H])c([2H])c([2H])c([2H])c3[2H])n2)c([2H])c1[2H]] | 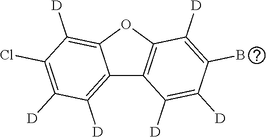 |
| 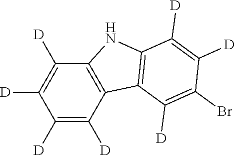 [SMILES: [2H]c1c([2H])c([2H])c2c([nH]c3c([2H])c([2H])c(Br)c([2H])c32)c1[2H]] | 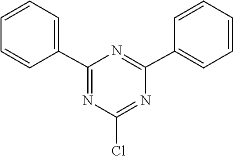 [SMILES: Clc1nc(-c2ccccc2)nc(-c2ccccc2)n1] | 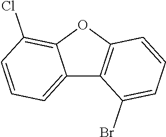 [SMILES: Clc1cccc2c1oc1cccc(Br)c12] |
| 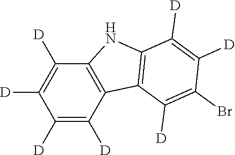 [SMILES: [2H]c1c([2H])c([2H])c2c([nH]c3c([2H])c([2H])c(Br)c([2H])c32)c1[2H]] | 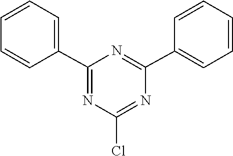 [SMILES: Clc1nc(-c2ccccc2)nc(-c2ccccc2)n1] | 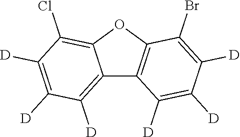 [SMILES: [2H]c1c([2H])c([2H])c2c(oc3c(Br)c([2H])c([2H])c([2H])c32)c1Cl] |
| 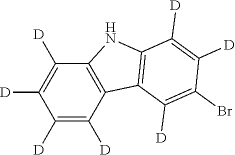 [SMILES: [2H]c1c([2H])c([2H])c2c([nH]c3c([2H])c([2H])c(Br)c([2H])c32)c1[2H]] | 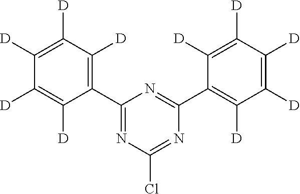 [SMILES: [2H]c1c([2H])c([2H])c(-c2nc(Cl)nc(-c3c([2H])c([2H])c([2H])c([2H])c3[2H])n2)c([2H])c1[2H]] | 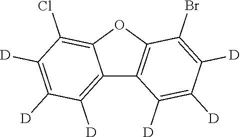 [SMILES: [2H]c1c([2H])c([2H])c2c(oc3c(Br)c([2H])c([2H])c([2H])c32)c1Cl] |
| 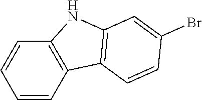 [SMILES: Brc1ccc2c(c1)[nH]c1ccccc12] | 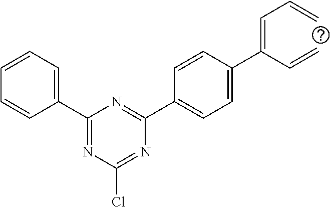 [SMILES: C=C/C=C(\C=C)c1ccc(-c2nc(Cl)nc(-c3ccccc3)n2)cc1] | 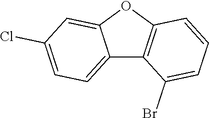 [SMILES: Clc1ccc2c(c1)oc1cccc(Br)c12] |
| 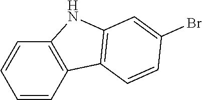 [SMILES: Brc1ccc2c(c1)[nH]c1ccccc12] | 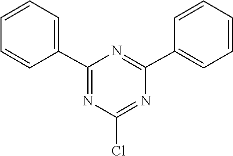 [SMILES: Clc1nc(-c2ccccc2)nc(-c2ccccc2)n1] | 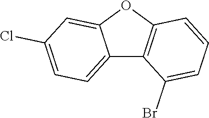 [SMILES: Clc1ccc2c(c1)oc1cccc(Br)c12] |
| 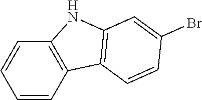 [SMILES: Brc1ccc2c(c1)[nH]c1ccccc12] | 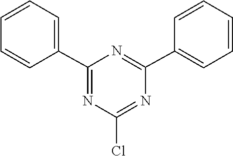 [SMILES: Clc1nc(-c2ccccc2)nc(-c2ccccc2)n1] | 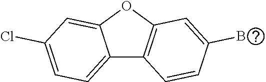 |
| 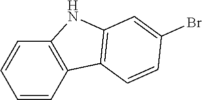 [SMILES: Brc1ccc2c(c1)[nH]c1ccccc12] | 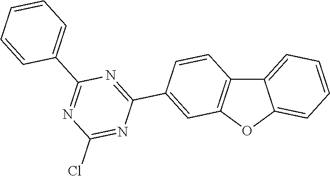 [SMILES: Clc1nc(-c2ccccc2)nc(-c2ccc3c(c2)oc2ccccc23)n1] | 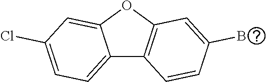 |

| Inter- | Target Compound |
|---|---|
| medi- | (Compound No., |
| ate D | Yield) |
| 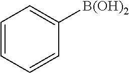 | 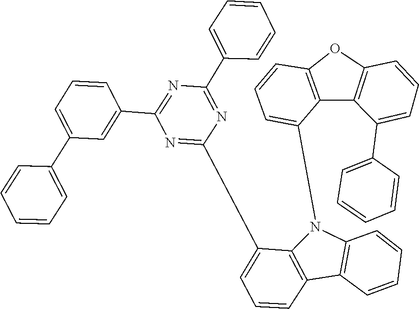 [SMILES: c1ccc(-c2cccc(-c3nc(-c4ccccc4)nc(-c4cccc5c6ccccc6n(-c6cccc7oc8cccc(-c9ccccc9)c8c67)c45)n3)c2)cc1] |
| | 1-3, 80% |
| 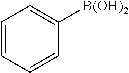 | 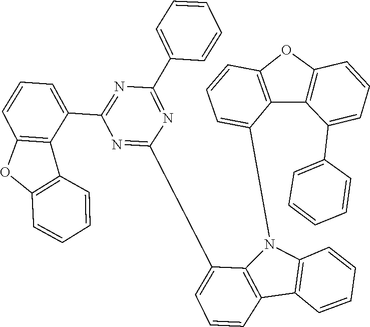 [SMILES: c1ccc(-c2nc(-c3cccc4oc5ccccc5c34)nc(-c3cccc4c5ccccc5n(-c5cccc6oc7cccc(-c8ccccc8)c7c56)c34)n2)cc1] |
| | 1-6, 78% |
| 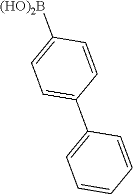 [SMILES: OB(O)c1ccc(-c2ccccc2)cc1] | 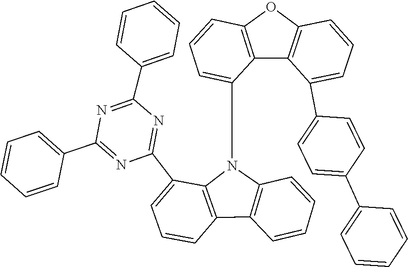 [SMILES: c1ccc(-c2ccc(-c3cccc4oc5cccc(-n6c7ccccc7c7cccc(-c8nc(-c9ccccc9)nc(-c9ccccc9)n8)c76)c5c34)cc2)cc1] |
| | 1-8, 73% |
| 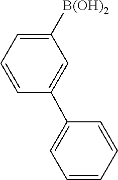 [SMILES: OB(O)c1cccc(-c2ccccc2)c1] | 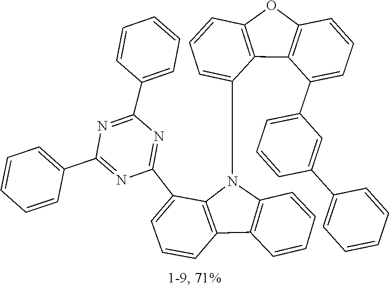 [SMILES: c1ccc(-c2cccc(-c3cccc4oc5cccc(CCn6c7ccccc7c7cccc(-c8nc(-c9ccccc9)nc(-c9ccccc9)n8)c76)c5c34)c2)cc1] |
| 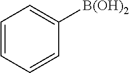 | 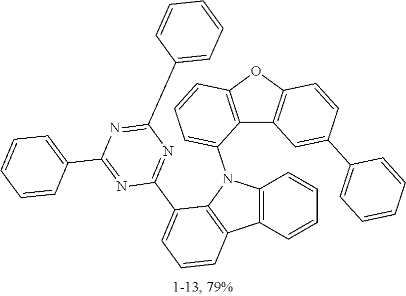 [SMILES: c1ccc(Cc2nc(-c3ccccc3)nc(-c3cccc4c5ccccc5n(-c5cccc6oc7ccc(-c8ccccc8)cc7c56)c34)n2)cc1] |
| 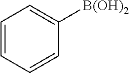 | 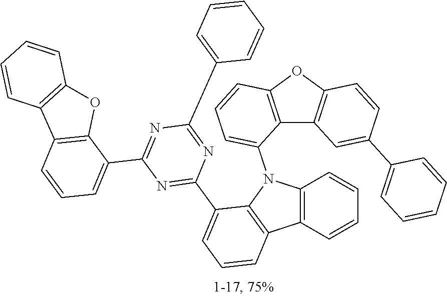 [SMILES: c1ccc(Cc2nc(-c3cccc4c3oc3ccccc34)nc(-c3cccc4c5ccccc5n(-c5cccc6oc7ccc(-c8ccccc8)cc7c56)c34)n2)cc1] |
| 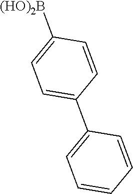 [SMILES: OB(O)c1ccc(-c2ccccc2)cc1] | 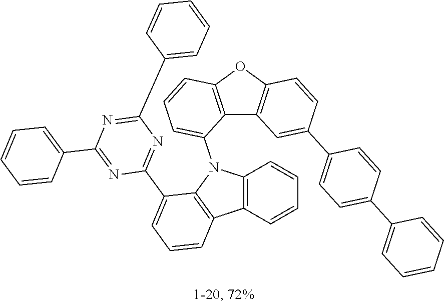 [SMILES: c1ccc(Cc2nc(-c3ccccc3)nc(-c3cccc4c5ccccc5n(-c5cccc6oc7ccc(-c8ccc(-c9ccccc9)cc8)cc7c56)c34)n2)cc1] |
| 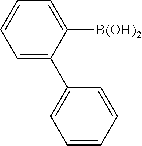 [SMILES: OB(O)c1ccccc1-c1ccccc1] | 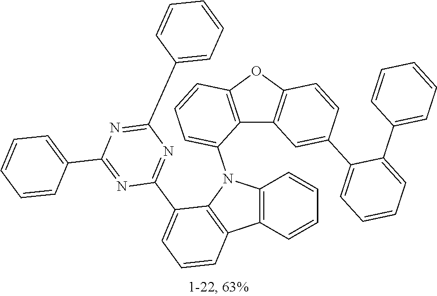 [SMILES: c1ccc(Cc2nc(-c3ccccc3)nc(-c3cccc4c5ccccc5n(-c5cccc6oc7ccc(-c8ccccc8-c8ccccc8)cc7c56)c34)n2)cc1] |
| 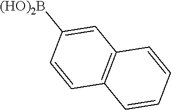 [SMILES: OB(O)c1ccc2ccccc2c1] | 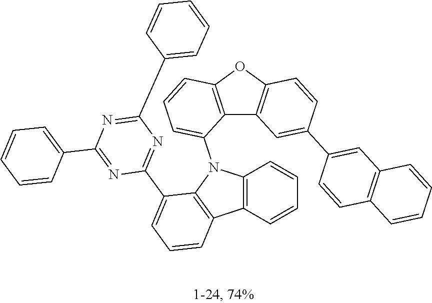 [SMILES: c1ccc(Cc2nc(-c3ccccc3)nc(-c3cccc4c5ccccc5n(-c5cccc6oc7ccc(-c8ccc9ccccc9c8)cc7c56)c34)n2)cc1] |
| 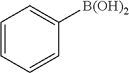 | 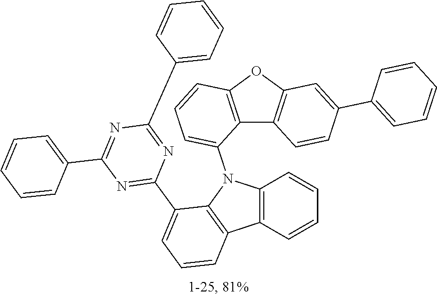 [SMILES: c1ccc(Cc2nc(-c3ccccc3)nc(-c3cccc4c5ccccc5n(-c5cccc6oc7cc(-c8ccccc8)ccc7c56)c34)n2)cc1] |
| 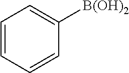 | 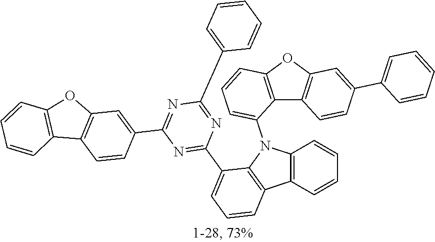 [SMILES: c1ccc(Cc2nc(-c3ccc4c(c3)oc3ccccc34)nc(-c3cccc4c5ccccc5n(-c5cccc6oc7cc(-c8ccccc8)ccc7c56)c34)n2)cc1] |
| 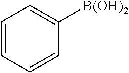 | 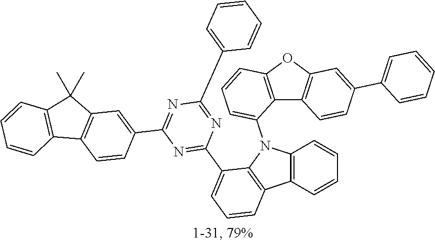 [SMILES: CC1(C)c2ccccc2-c2ccc(-c3nc(Cc4ccccc4)nc(-c4cccc5c6ccccc6n(-c6cccc7oc8cc(-c9ccccc9)ccc8c67)c45)n3)cc21] |
| 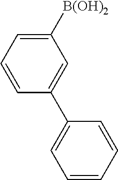 [SMILES: OB(O)c1cccc(-c2ccccc2)c1] | 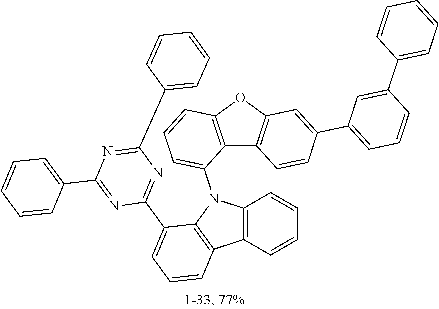 [SMILES: c1ccc(Cc2nc(-c3ccccc3)nc(-c3cccc4c5ccccc5n(-c5cccc6oc7cc(-c8cccc(-c9ccccc9)c8)ccc7c56)c34)n2)cc1] |
| 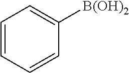 | 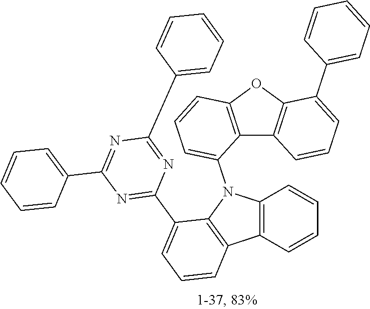 [SMILES: c1ccc(Cc2nc(-c3ccccc3)nc(-c3cccc4c5ccccc5n(-c5cccc6oc7c(-c8ccccc8)cccc7c56)c34)n2)cc1] |
| 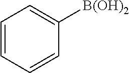 | 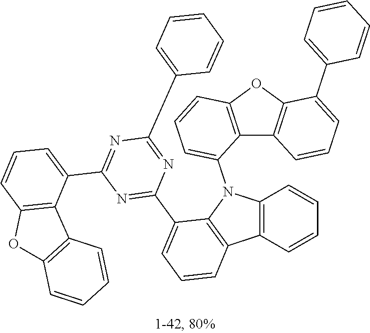 [SMILES: c1ccc(Cc2nc(-c3cccc4oc5ccccc5c34)nc(-c3cccc4c5ccccc5n(-c5cccc6oc7c(-c8ccccc8)cccc7c56)c34)n2)cc1] |
| 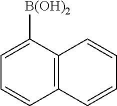 [SMILES: OB(O)c1cccc2ccccc12] | 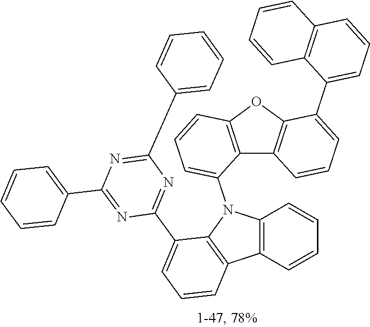 [SMILES: c1ccc(Cc2nc(-c3ccccc3)nc(-c3cccc4c5ccccc5n(-c5cccc6oc7c(-c8cccc9ccccc89)cccc7c56)c34)n2)cc1] |
| 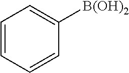 | 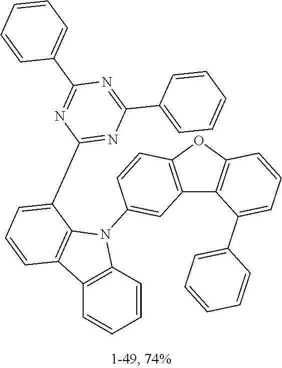 [SMILES: c1ccc(-c2nc(Cc3cccc4c5ccccc5n(-c5ccc6oc7cccc(-c8ccccc8)c7c6c5)c34)nc(-c3ccccc3)n2)cc1] |
| 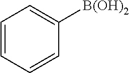 | 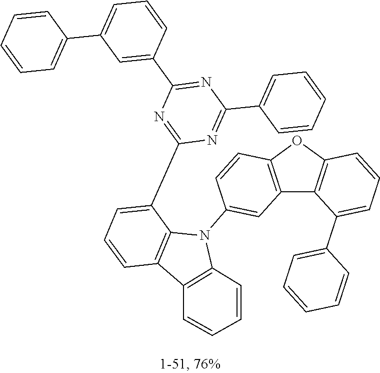 [SMILES: c1ccc(-c2cccc(-c3nc(Cc4cccc5c6ccccc6n(-c6ccc7oc8cccc(-c9ccccc9)c8c7c6)c45)nc(-c4ccccc4)n3)c2)cc1] |
| 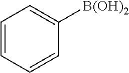 | 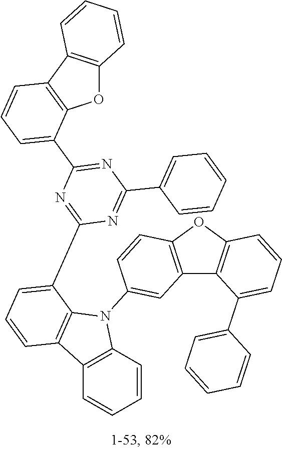 [SMILES: c1ccc(-c2nc(Cc3cccc4c5ccccc5n(-c5ccc6oc7cccc(-c8ccccc8)c7c6c5)c34)nc(-c3cccc4c3oc3ccccc34)n2)cc1] |
| 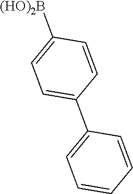 [SMILES: OB(O)c1ccc(-c2ccccc2)cc1] | 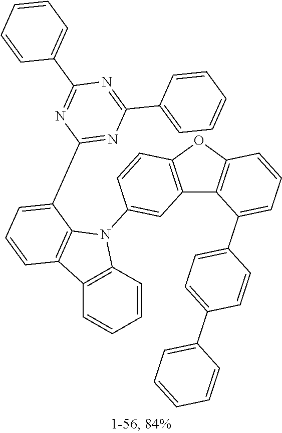 [SMILES: c1ccc(-c2ccc(-c3cccc4oc5ccc(-n6c7ccccc7c7cccc(Cc8nc(-c9ccccc9)nc(-c9ccccc9)n8)c76)cc5c34)cc2)cc1] |
| 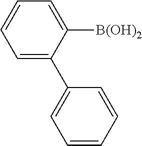 [SMILES: OB(O)c1ccccc1-c1ccccc1] | 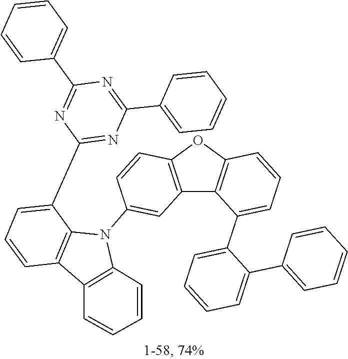 [SMILES: c1ccc(-c2nc(Cc3cccc4c5ccccc5n(-c5ccc6oc7cccc(-c8ccccc8-c8ccccc8)c7c6c5)c34)nc(-c3ccccc3)n2)cc1] |
| 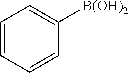 | 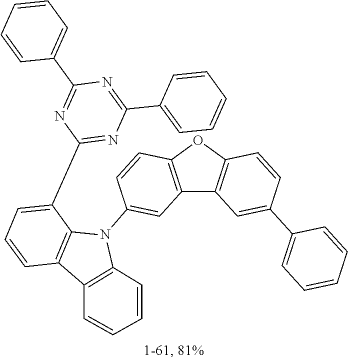 [SMILES: c1ccc(-c2ccc3oc4ccc(-n5c6ccccc6c6cccc(Cc7nc(-c8ccccc8)nc(-c8ccccc8)n7)c65)cc4c3c2)cc1] |
| 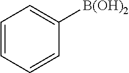 | 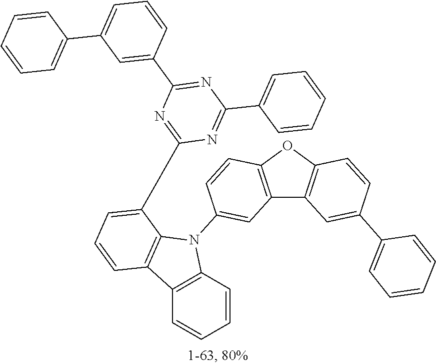 [SMILES: c1ccc(-c2cccc(-c3nc(-c4ccccc4)nc(-c4cccc5c6ccccc6n(-c6ccc7oc8ccc(-c9ccccc9)cc8c7c6)c45)n3)c2)cc1] |
| 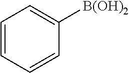 | 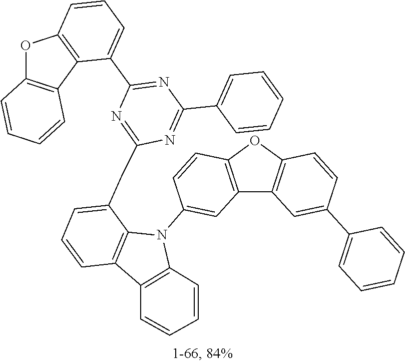 [SMILES: c1ccc(-c2ccc3oc4ccc(-n5c6ccccc6c6cccc(Cc7nc(-c8ccccc8)nc(-c8cccc9oc%10ccccc%10c89)n7)c65)cc4c3c2)cc1] |
| 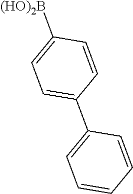 [SMILES: OB(O)c1ccc(-c2ccccc2)cc1] | 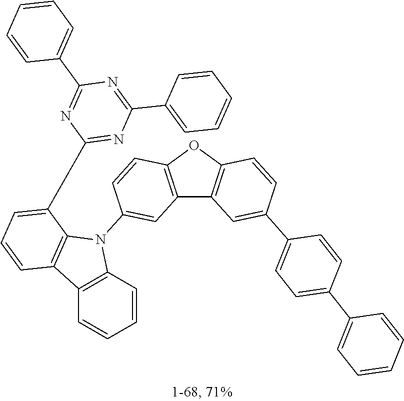 [SMILES: c1ccc(-c2ccc(-c3ccc4oc5ccc(-n6c7ccccc7c7cccc(-c8nc(-c9ccccc9)nc(-c9ccccc9)n8)c76)cc5c4c3)cc2)cc1] |
| 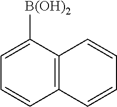 [SMILES: OB(O)c1cccc2ccccc12] | 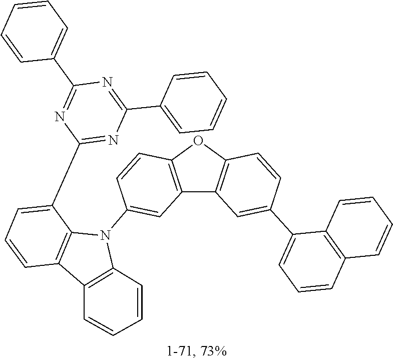 [SMILES: c1ccc(-c2nc(Cc3cccc4c5ccccc5n(-c5ccc6oc7ccc(-c8cccc9ccccc89)cc7c6c5)c34)nc(-c3ccccc3)n2)cc1] |
| 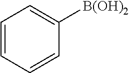 | 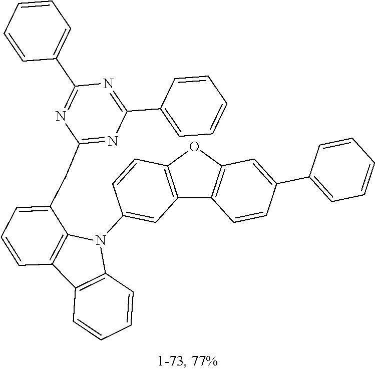 [SMILES: c1ccc(-c2ccc3c(c2)oc2ccc(-n4c5ccccc5c5cccc(Cc6nc(-c7ccccc7)nc(-c7ccccc7)n6)c54)cc23)cc1] |
| 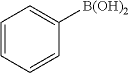 | 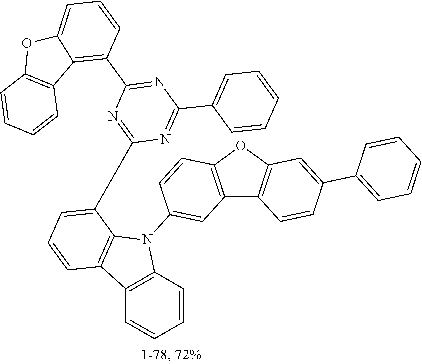 [SMILES: c1ccc(-c2ccc3c(c2)oc2ccc(-n4c5ccccc5c5cccc(-c6nc(-c7ccccc7)nc(-c7cccc8oc9ccccc9c78)n6)c54)cc23)cc1] |
| 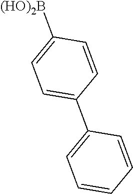 [SMILES: OB(O)c1ccc(-c2ccccc2)cc1] | 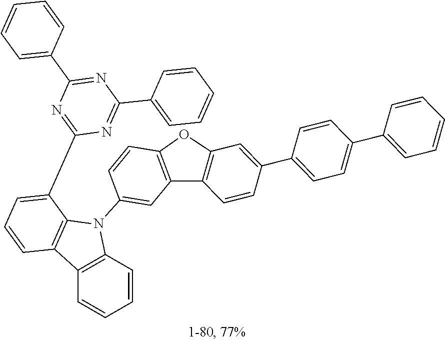 [SMILES: c1ccc(-c2ccc(-c3ccc4c(c3)oc3ccc(-n5c6ccccc6c6cccc(-c7nc(-c8ccccc8)nc(-c8ccccc8)n7)c65)cc34)cc2)cc1] |
| 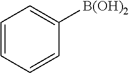 | 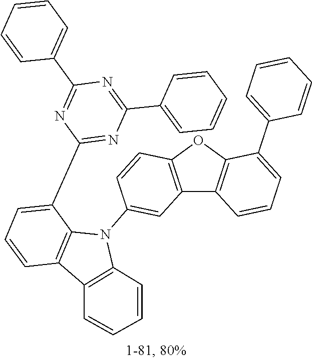 [SMILES: c1ccc(-c2nc(Cc3cccc4c5ccccc5n(-c5ccc6oc7c(-c8ccccc8)cccc7c6c5)c34)nc(-c3ccccc3)n2)cc1] |
| 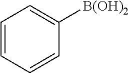 | 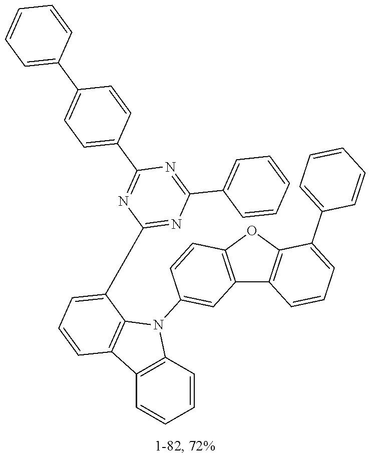 [SMILES: c1ccc(-c2ccc(-c3nc(-c4ccccc4)nc(-c4cccc5c6ccccc6n(-c6ccc7oc8c(-c9ccccc9)cccc8c7c6)c45)n3)cc2)cc1] |
| 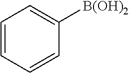 | 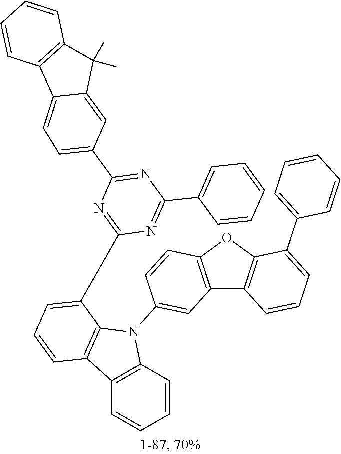 [SMILES: CC1(C)c2ccccc2-c2ccc(-c3nc(-c4ccccc4)nc(-c4cccc5c6ccccc6n(-c6ccc7oc8c(-c9ccccc9)cccc8c7c6)c45)n3)cc21] |
| 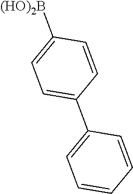 [SMILES: OB(O)c1ccc(-c2ccccc2)cc1] | 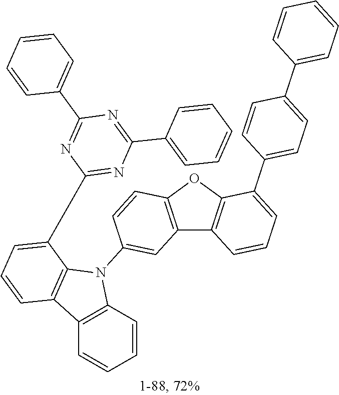 [SMILES: c1ccc(-c2ccc(-c3cccc4c3oc3ccc(-n5c6ccccc6c6cccc(-c7nc(-c8ccccc8)nc(-c8ccccc8)n7)c65)cc34)cc2)cc1] |
| 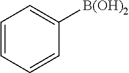 | 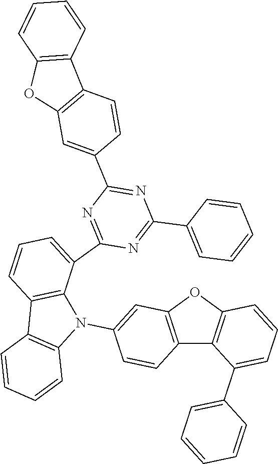 [SMILES: c1ccc(-c2nc(-c3ccc4c(c3)oc3ccccc34)nc(-c3cccc4c5ccccc5n(-c5ccc6c(c5)oc5cccc(-c7ccccc7)c56)c34)n2)cc1] |
| | 1-92, 73% |
| 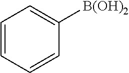 | 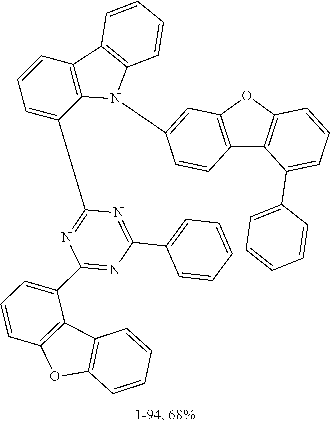 [SMILES: c1ccc(-c2nc(-c3cccc4oc5ccccc5c34)nc(-c3cccc4c5ccccc5n(-c5ccc6c(c5)oc5cccc(-c7ccccc7)c56)c34)n2)cc1] |
| 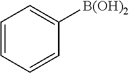 | 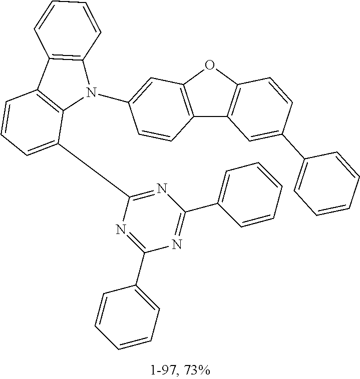 [SMILES: c1ccc(-c2ccc3oc4cc(-n5c6ccccc6c6cccc(-c7nc(-c8ccccc8)nc(-c8ccccc8)n7)c65)ccc4c3c2)cc1] |
| 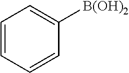 | 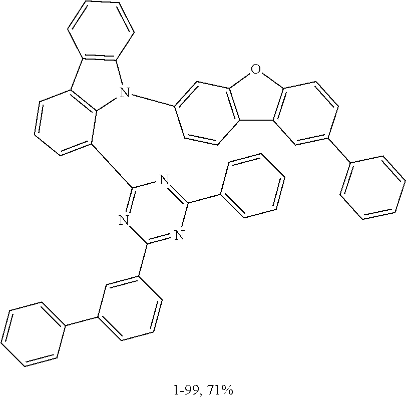 [SMILES: c1ccc(-c2cccc(-c3nc(-c4ccccc4)nc(-c4cccc5c6ccccc6n(-c6ccc7c(c6)oc6ccc(-c8ccccc8)cc67)c45)n3)c2)cc1] |
| 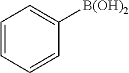 | 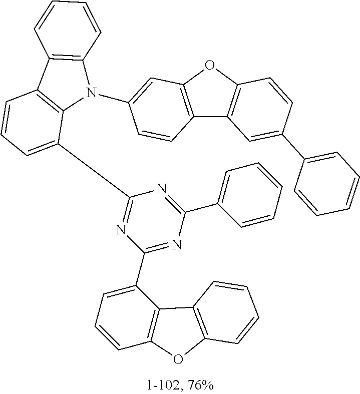 [SMILES: c1ccc(-c2ccc3oc4cc(-n5c6ccccc6c6cccc(-c7nc(-c8ccccc8)nc(-c8cccc9oc%10ccccc%10c89)n7)c65)ccc4c3c2)cc1] |
| 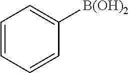 | 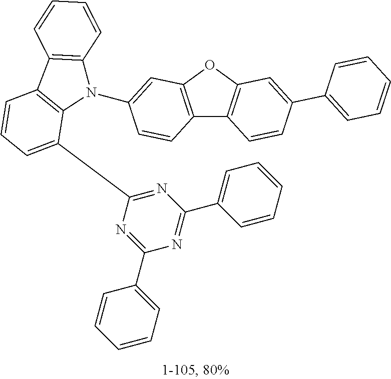 [SMILES: c1ccc(-c2ccc3c(c2)oc2cc(-n4c5ccccc5c5cccc(-c6nc(-c7ccccc7)nc(-c7ccccc7)n6)c54)ccc23)cc1] |
| 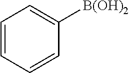 | 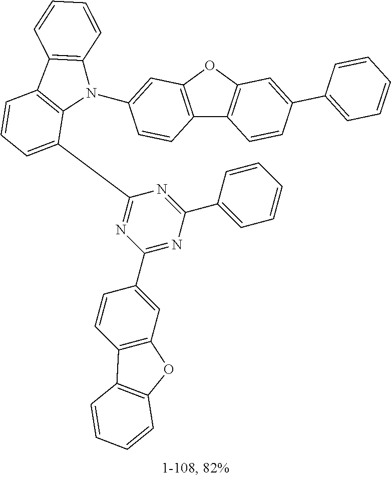 [SMILES: c1ccc(-c2ccc3c(c2)oc2cc(-n4c5ccccc5c5cccc(-c6nc(-c7ccccc7)nc(-c7ccc8c(c7)oc7ccccc78)n6)c54)ccc23)cc1] |
| 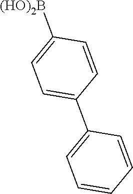 [SMILES: OB(O)c1ccc(-c2ccccc2)cc1] | 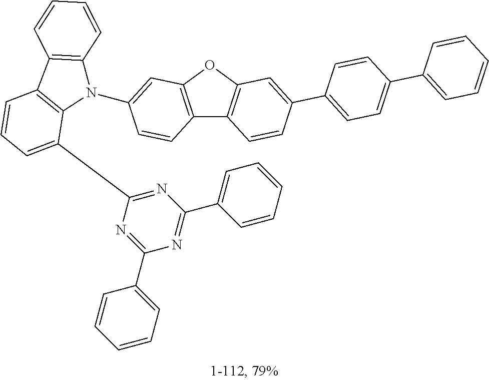 [SMILES: c1ccc(-c2ccc(-c3ccc4c(c3)oc3cc(-n5c6ccccc6c6cccc(-c7nc(-c8ccccc8)nc(-c8ccccc8)n7)c65)ccc34)cc2)cc1] |
| 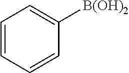 | 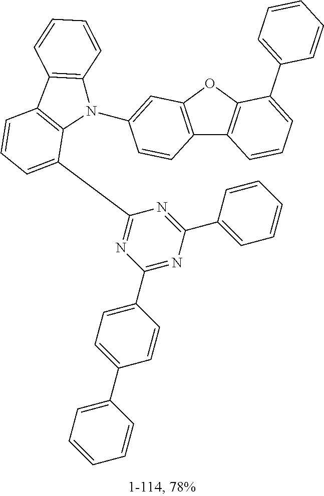 [SMILES: c1ccc(-c2ccc(-c3nc(-c4ccccc4)nc(-c4cccc5c6ccccc6n(-c6ccc7c(c6)oc6c(-c8ccccc8)cccc67)c45)n3)cc2)cc1] |
| 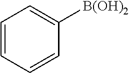 | 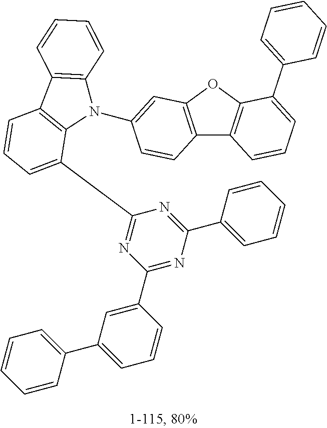 [SMILES: c1ccc(-c2cccc(-c3nc(-c4ccccc4)nc(-c4cccc5c6ccccc6n(-c6ccc7c(c6)oc6c(-c8ccccc8)cccc67)c45)n3)c2)cc1] |
| 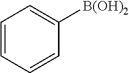 | 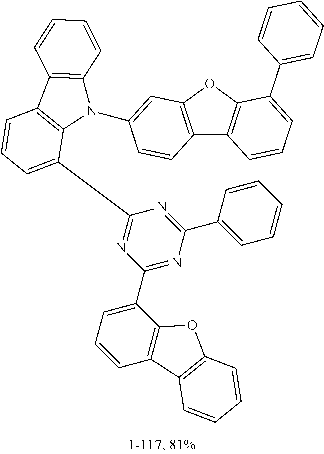 [SMILES: c1ccc(-c2nc(-c3cccc4c3oc3ccccc34)nc(-c3cccc4c5ccccc5n(-c5ccc6c(c5)oc5c(-c7ccccc7)cccc56)c34)n2)cc1] |
| 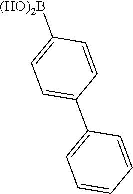 [SMILES: OB(O)c1ccc(-c2ccccc2)cc1] | 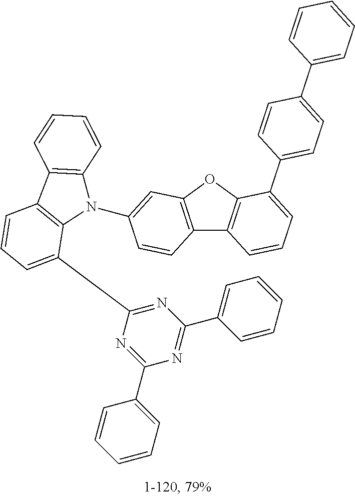 [SMILES: c1ccc(-c2ccc(-c3cccc4c3oc3cc(-n5c6ccccc6c6cccc(-c7nc(-c8ccccc8)nc(-c8ccccc8)n7)c65)ccc34)cc2)cc1] |
| 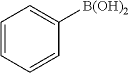 | 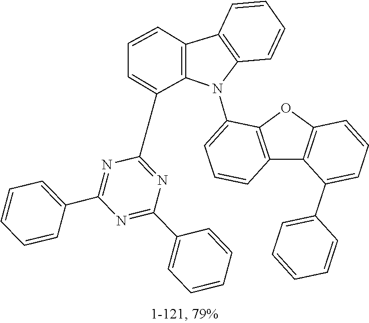 [SMILES: c1ccc(-c2nc(-c3ccccc3)nc(-c3cccc4c5ccccc5n(-c5cccc6c5oc5cccc(-c7ccccc7)c56)c34)n2)cc1] |
| 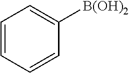 | 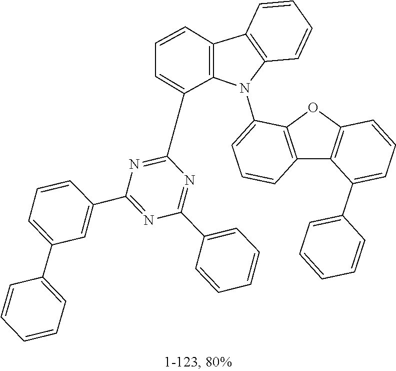 [SMILES: c1ccc(-c2cccc(-c3nc(-c4ccccc4)nc(-c4cccc5c6ccccc6n(-c6cccc7c6oc6cccc(-c8ccccc8)c67)c45)n3)c2)cc1] |
| 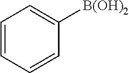 | 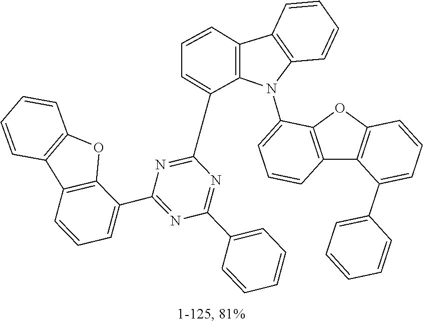 [SMILES: c1ccc(-c2nc(-c3cccc4c3oc3ccccc34)nc(-c3cccc4c5ccccc5n(-c5cccc6c5oc5cccc(-c7ccccc7)c56)c34)n2)cc1] |
| 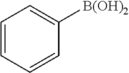 | 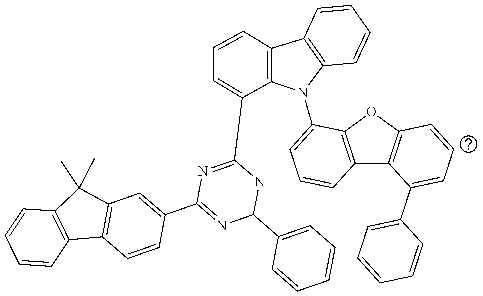 |
| | 1-127, 78% |
| 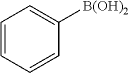 | 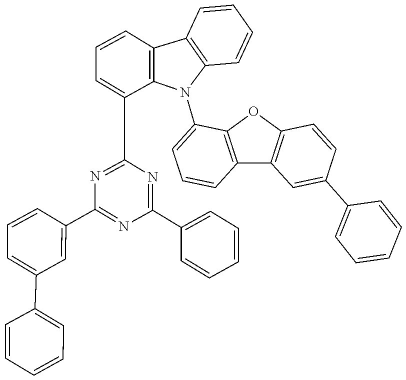 [SMILES: c1ccc(-c2cccc(-c3nc(-c4ccccc4)nc(-c4cccc5c6ccccc6n(-c6cccc7c6oc6ccc(-c8ccccc8)cc67)c45)n3)c2)cc1] |
| | 1-131, 77% |
| 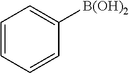 | 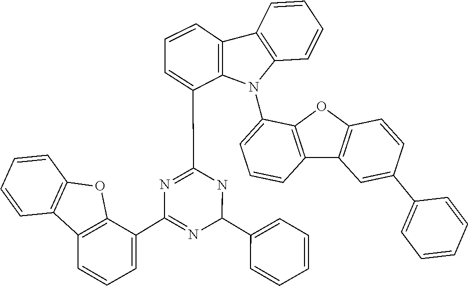 |
| | 1-133, 73% |
| 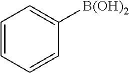 | 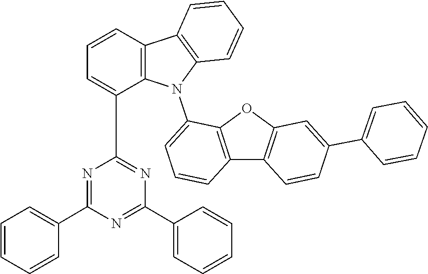 [SMILES: c1ccc(-c2ccc3c(c2)oc2c(-n4c5ccccc5c5cccc(-c6nc(-c7ccccc7)nc(-c7ccccc7)n6)c54)cccc23)cc1] |
| | 1-137, 75% |
| 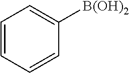 | 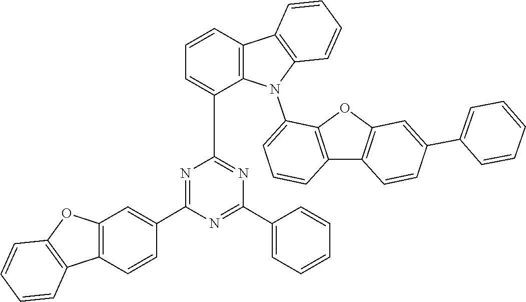 [SMILES: c1ccc(-c2ccc3c(c2)oc2c(-n4c5ccccc5c5cccc(-c6nc(-c7ccccc7)nc(-c7ccc8c(c7)oc7ccccc78)n6)c54)cccc23)cc1] |
| | 1-140, 72% |
| 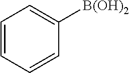 | 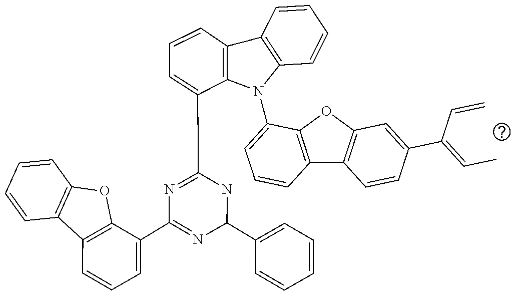 |
| | 1-141, 70% |
| 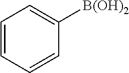 | 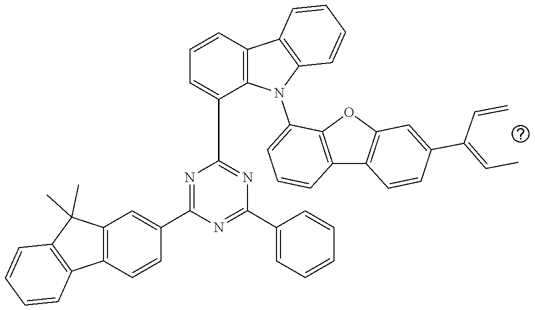 [SMILES: C=C/C(=C\C)c1ccc2c(c1)oc1c(-n3c4ccccc4c4cccc(-c5nc(-c6ccccc6)nc(-c6ccc7c(c6)C(C)(C)c6ccccc6-7)n5)c43)cccc12] |
| | 1-143, 77% |
| 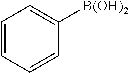 | 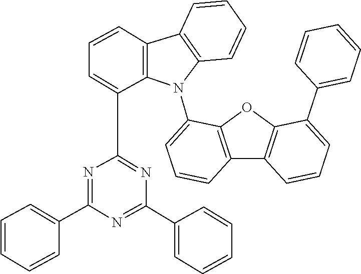 [SMILES: c1ccc(-c2nc(-c3ccccc3)nc(-c3cccc4c5ccccc5n(-c5cccc6c5oc5c(-c7ccccc7)cccc56)c34)n2)cc1] |
| | 1-145, 72% |
| 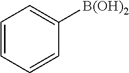 | 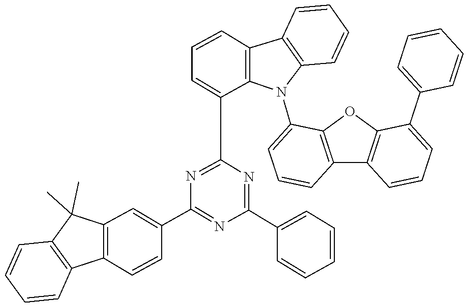 [SMILES: CC1(C)c2ccccc2-c2ccc(-c3nc(-c4ccccc4)nc(-c4cccc5c6ccccc6n(-c6cccc7c6oc6c(-c8ccccc8)cccc67)c45)n3)cc21] |
| | 1-151, 76% |
| 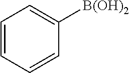 | 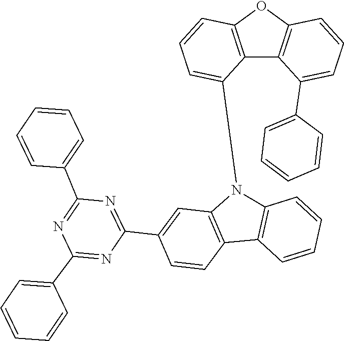 [SMILES: c1ccc(-c2nc(-c3ccccc3)nc(-c3ccc4c5ccccc5n(-c5cccc6oc7cccc(-c8ccccc8)c7c56)c4c3)n2)cc1] |
| | 1-153, 75% |
| 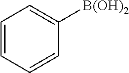 | 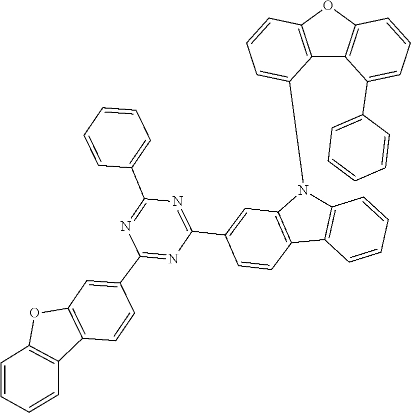 [SMILES: c1ccc(-c2nc(-c3ccc4c(c3)oc3ccccc34)nc(-c3ccc4c5ccccc5n(-c5cccc6oc7cccc(-c8ccccc8)c7c56)c4c3)n2)cc1] |
| | 1-156, 81% |
| 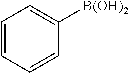 | 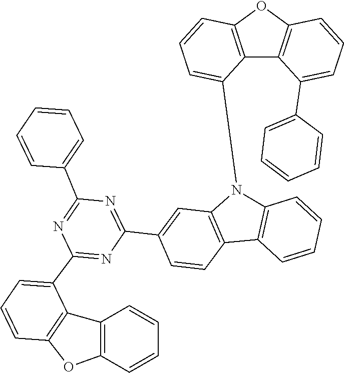 [SMILES: c1ccc(-c2nc(-c3ccc4c5ccccc5n(-c5cccc6oc7cccc(-c8ccccc8)c7c56)c4c3)nc(-c3cccc4oc5ccccc5c34)n2)cc1] |
| | 1-158, 78% |
| 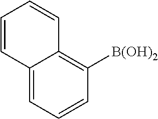 [SMILES: OB(O)c1cccc2ccccc12] | 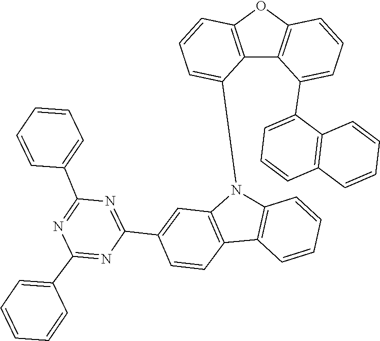 [SMILES: c1ccc(-c2nc(-c3ccccc3)nc(-c3ccc4c5ccccc5n(-c5cccc6oc7cccc(-c8cccc9ccccc89)c7c56)c4c3)n2)cc1] |
| | 1-163, 77% |
| 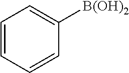 | 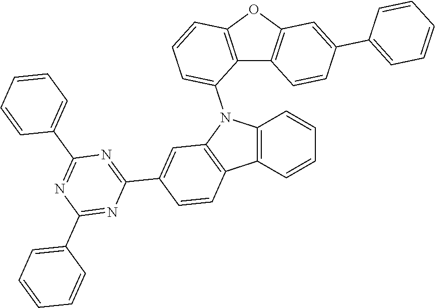 [SMILES: c1ccc(-c2ccc3c(c2)oc2cccc(-n4c5ccccc5c5ccc(-c6nc(-c7ccccc7)nc(-c7ccccc7)n6)cc54)c23)cc1] |
| | 1-165, 79% |
| 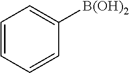 | 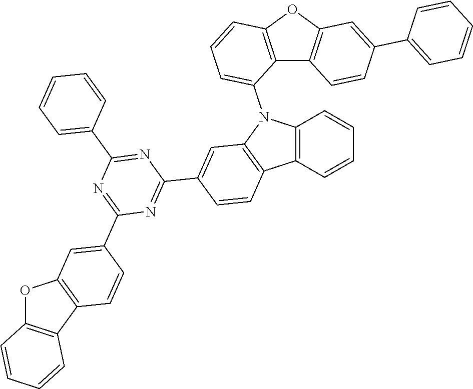 [SMILES: c1ccc(-c2ccc3c(c2)oc2cccc(-n4c5ccccc5c5ccc(-c6nc(-c7ccccc7)nc(-c7ccc8c(c7)oc7ccccc78)n6)cc54)c23)cc1] |
| | 1-168, 82% |
| 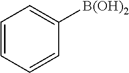 | 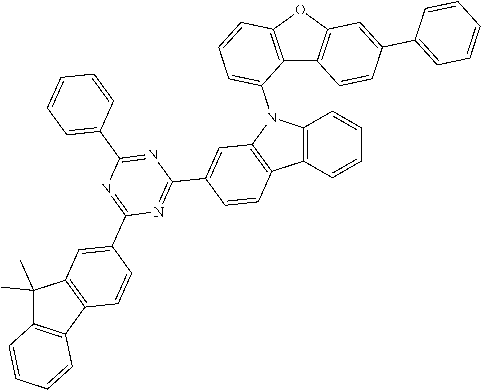 [SMILES: CC1(C)c2ccccc2-c2ccc(-c3nc(-c4ccccc4)nc(-c4ccc5c6ccccc6n(-c6cccc7oc8cc(-c9ccccc9)ccc8c67)c5c4)n3)cc21] |
| | 1-171, 75% |
| 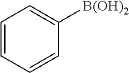 | 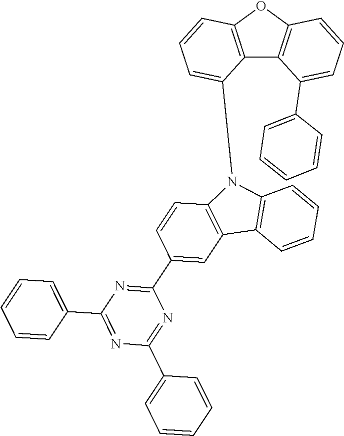 [SMILES: c1ccc(-c2nc(-c3ccccc3)nc(-c3ccc4c(c3)c3ccccc3n4-c3cccc4oc5cccc(-c6ccccc6)c5c34)n2)cc1] |
| | 1-173, 83% |
| 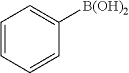 | 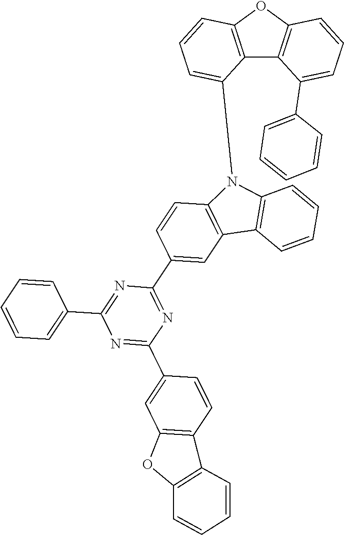 [SMILES: c1ccc(-c2nc(-c3ccc4c(c3)oc3ccccc34)nc(-c3ccc4c(c3)c3ccccc3n4-c3cccc4oc5cccc(-c6ccccc6)c5c34)n2)cc1] |
| | 1-176, 80% |
| 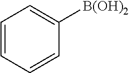 | 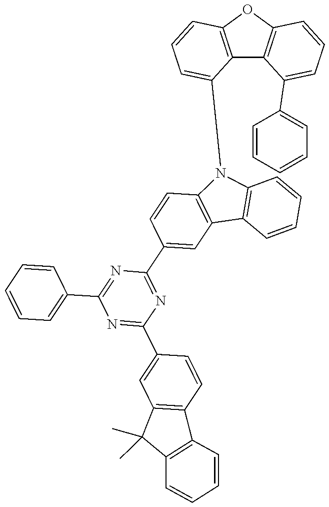 [SMILES: CC1(C)c2ccccc2-c2ccc(-c3nc(-c4ccccc4)nc(-c4ccc5c(c4)c4ccccc4n5-c4cccc5oc6cccc(-c7ccccc7)c6c45)n3)cc21] |
| | 1-179, 71% |
| 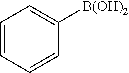 | 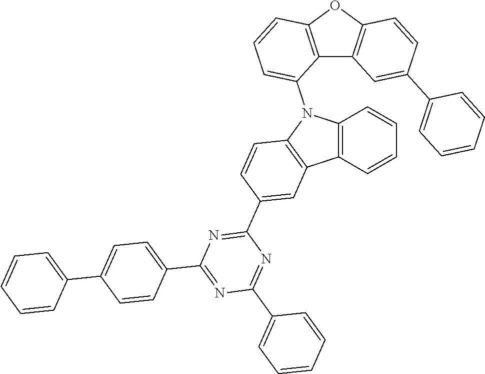 [SMILES: c1ccc(-c2ccc(-c3nc(-c4ccccc4)nc(-c4ccc5c(c4)c4ccccc4n5-c4cccc5oc6ccc(-c7ccccc7)cc6c45)n3)cc2)cc1] |
| | 1-182, 82% |
| 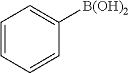 | 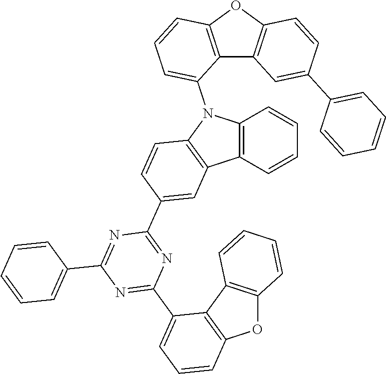 [SMILES: c1ccc(-c2ccc3oc4cccc(-n5c6ccccc6c6cc(-c7nc(-c8ccccc8)nc(-c8cccc9oc%10ccccc%10c89)n7)ccc65)c4c3c2)cc1] |
| | 1-186, 81% |
| 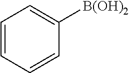 | 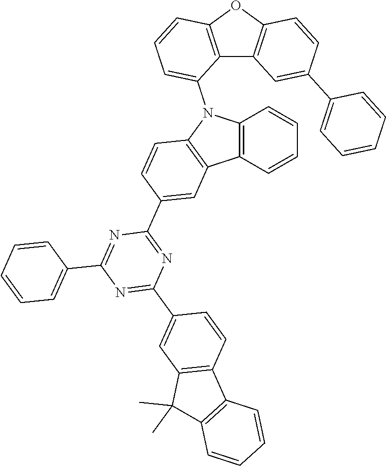 [SMILES: CC1(C)c2ccccc2-c2ccc(-c3nc(-c4ccccc4)nc(-c4ccc5c(c4)c4ccccc4n5-c4cccc5oc6ccc(-c7ccccc7)cc6c45)n3)cc21] |
| | 1-187, 70% |
| 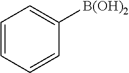 | 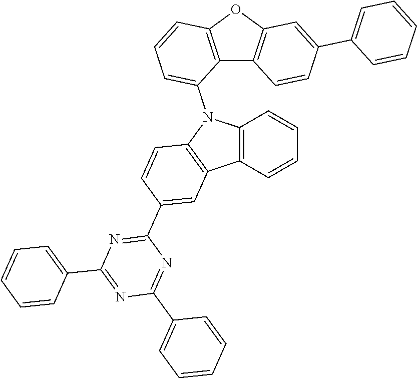 [SMILES: c1ccc(-c2ccc3c(c2)oc2cccc(-n4c5ccccc5c5cc(-c6nc(-c7ccccc7)nc(-c7ccccc7)n6)ccc54)c23)cc1] |
| | 1-189, 79% |
| 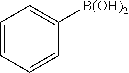 | 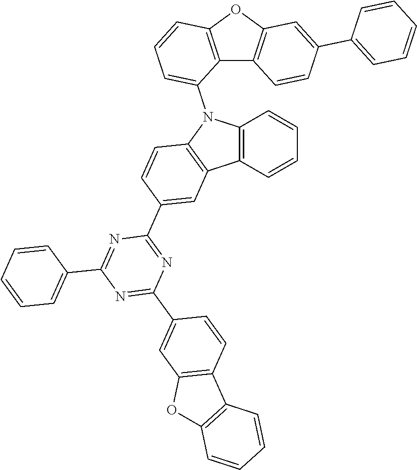 [SMILES: c1ccc(-c2ccc3c(c2)oc2cccc(-n4c5ccccc5c5cc(-c6nc(-c7ccccc7)nc(-c7ccc8c(c7)oc7ccccc78)n6)ccc54)c23)cc1] |
| | 1-192, 73% |
| 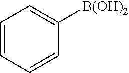 | 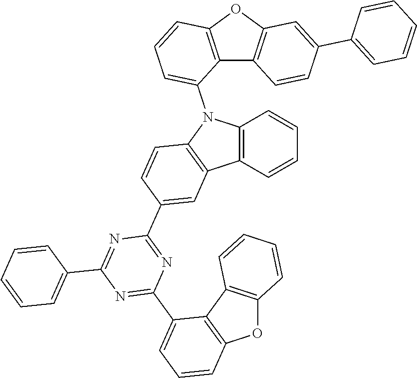 [SMILES: c1ccc(-c2ccc3c(c2)oc2cccc(-n4c5ccccc5c5cc(-c6nc(-c7ccccc7)nc(-c7cccc8oc9ccccc9c78)n6)ccc54)c23)cc1] |
| | 1-194, 77% |
| 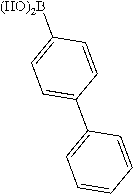 [SMILES: OB(O)c1ccc(-c2ccccc2)cc1] | 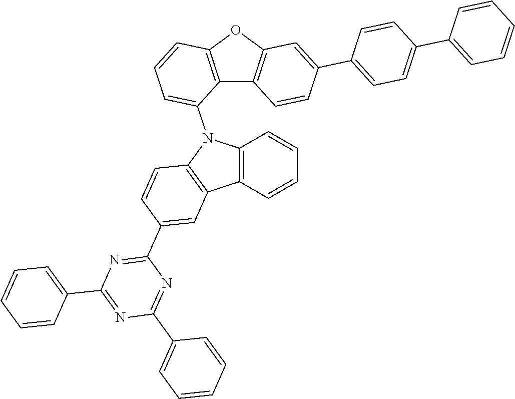 [SMILES: c1ccc(-c2ccc(-c3ccc4c(c3)oc3cccc(-n5c6ccccc6c6cc(-c7nc(-c8ccccc8)nc(-c8ccccc8)n7)ccc65)c34)cc2)cc1] |
| | 1-196, 76% |
| 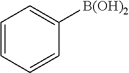 | 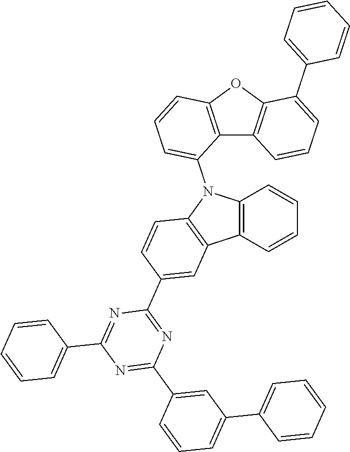 [SMILES: c1ccc(-c2cccc(-c3nc(-c4ccccc4)nc(-c4ccc5c(c4)c4ccccc4n5-c4cccc5oc6c(-c7ccccc7)cccc6c45)n3)c2)cc1] |
| | 1-199, 77% |
| 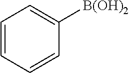 | 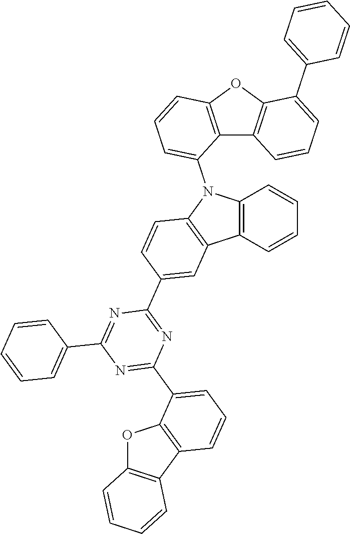 [SMILES: c1ccc(-c2nc(-c3ccc4c(c3)c3ccccc3n4-c3cccc4oc5c(-c6ccccc6)cccc5c34)nc(-c3cccc4c3oc3ccccc34)n2)cc1] |
| | 1-201, 72% |
| 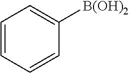 | 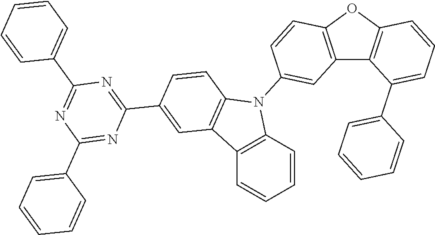 [SMILES: c1ccc(-c2nc(-c3ccccc3)nc(-c3ccc4c(c3)c3ccccc3n4-c3ccc4oc5cccc(-c6ccccc6)c5c4c3)n2)cc1] |
| | 1-205, 74% |
| 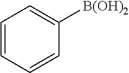 | 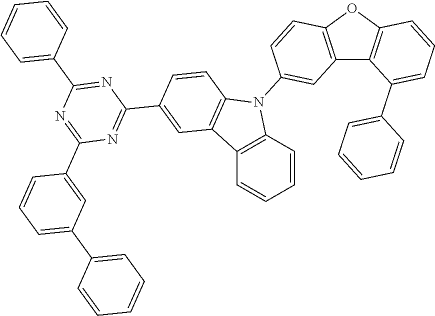 [SMILES: c1ccc(-c2cccc(-c3nc(-c4ccccc4)nc(-c4ccc5c(c4)c4ccccc4n5-c4ccc5oc6cccc(-c7ccccc7)c6c5c4)n3)c2)cc1] |
| | 1-207, 75% |
| 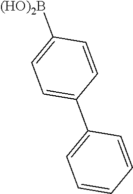 [SMILES: OB(O)c1ccc(-c2ccccc2)cc1] | 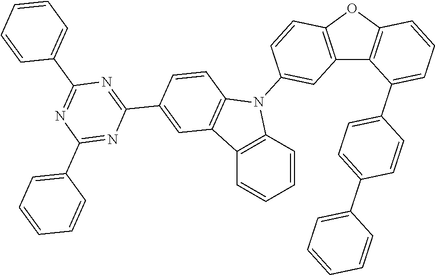 [SMILES: c1ccc(-c2ccc(-c3cccc4oc5ccc(-n6c7ccccc7c7cc(-c8nc(-c9ccccc9)nc(-c9ccccc9)n8)ccc76)cc5c34)cc2)cc1] |
| | 1-212, 77% |
| 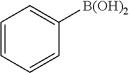 | 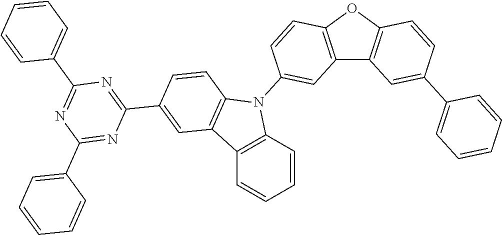 [SMILES: c1ccc(-c2ccc3oc4ccc(-n5c6ccccc6c6cc(-c7nc(-c8ccccc8)nc(-c8ccccc8)n7)ccc65)cc4c3c2)cc1] |
| | 1-213, 82% |
| 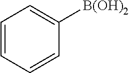 | 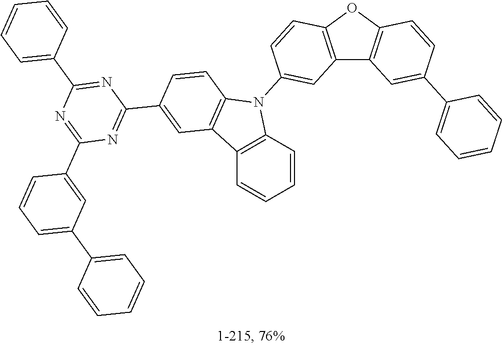 [SMILES: c1ccc(-c2cccc(-c3nc(-c4ccccc4)nc(-c4ccc5c(c4)c4ccccc4n5-c4ccc5oc6ccc(-c7ccccc7)cc6c5c4)n3)c2)cc1] |
| 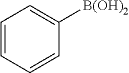 | 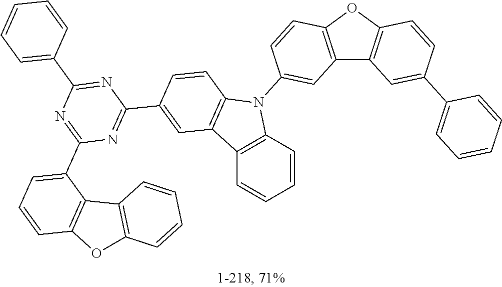 [SMILES: c1ccc(-c2ccc3oc4ccc(-n5c6ccccc6c6cc(-c7nc(-c8ccccc8)nc(-c8cccc9oc%10ccccc%10c89)n7)ccc65)cc4c3c2)cc1] |
| 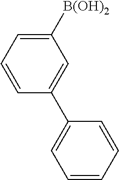 [SMILES: OB(O)c1cccc(-c2ccccc2)c1] | 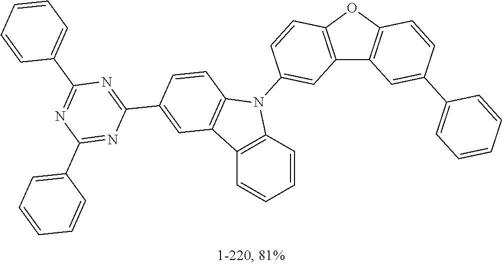 [SMILES: c1ccc(-c2ccc3oc4ccc(-n5c6ccccc6c6cc(-c7nc(-c8ccccc8)nc(-c8ccccc8)n7)ccc65)cc4c3c2)cc1] |
| 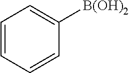 | 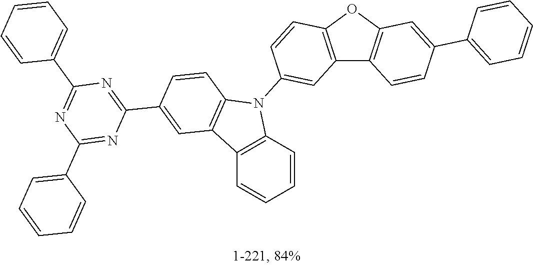 [SMILES: c1ccc(-c2ccc3c(c2)oc2ccc(-n4c5ccccc5c5cc(-c6nc(-c7ccccc7)nc(-c7ccccc7)n6)ccc54)cc23)cc1] |
| 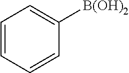 | 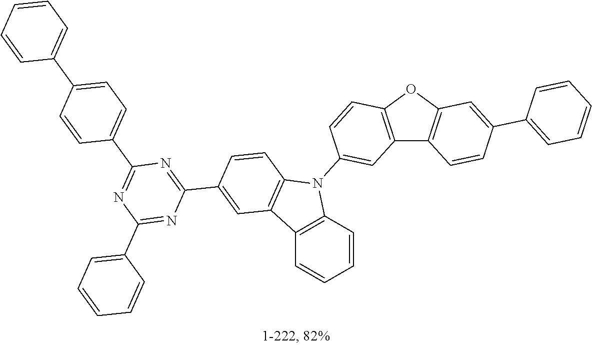 [SMILES: c1ccc(-c2ccc(-c3nc(-c4ccccc4)nc(-c4ccc5c(c4)c4ccccc4n5-c4ccc5oc6cc(-c7ccccc7)ccc6c5c4)n3)cc2)cc1] |
| 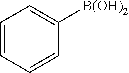 | 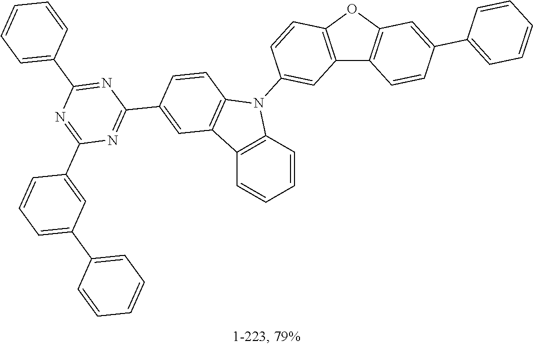 [SMILES: c1ccc(-c2cccc(-c3nc(-c4ccccc4)nc(-c4ccc5c(c4)c4ccccc4n5-c4ccc5oc6cc(-c7ccccc7)ccc6c5c4)n3)c2)cc1] |
| 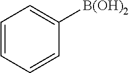 | 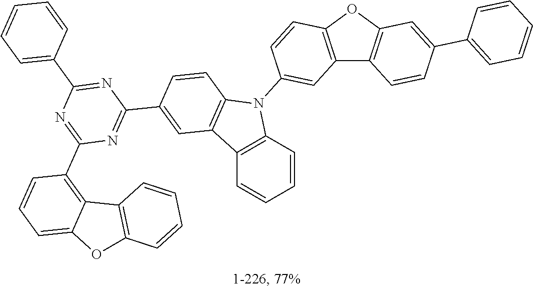 [SMILES: c1ccc(-c2ccc3c(c2)oc2ccc(-n4c5ccccc5c5cc(-c6nc(-c7ccccc7)nc(-c7cccc8oc9ccccc9c78)n6)ccc54)cc23)cc1] |
| 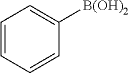 | 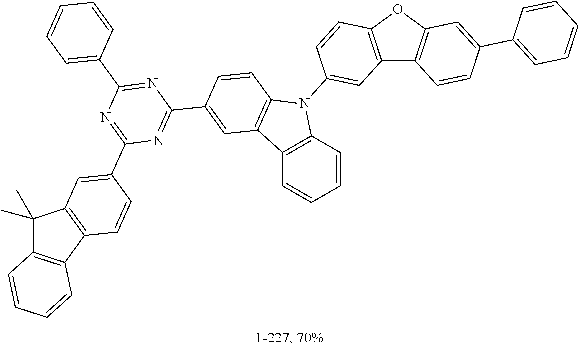 [SMILES: CC1(C)c2ccccc2-c2ccc(-c3nc(-c4ccccc4)nc(-c4ccc5c(c4)c4ccccc4n5-c4ccc5oc6cc(-c7ccccc7)ccc6c5c4)n3)cc21] |
| 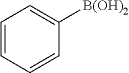 | 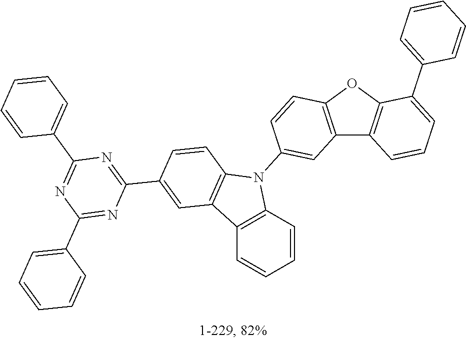 [SMILES: c1ccc(-c2nc(-c3ccccc3)nc(-c3ccc4c(c3)c3ccccc3n4-c3ccc4oc5c(-c6ccccc6)cccc5c4c3)n2)cc1] |
| 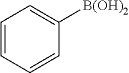 | 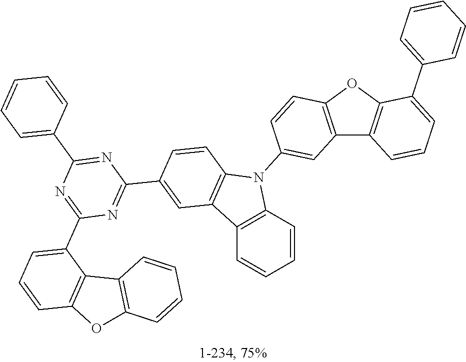 [SMILES: c1ccc(-c2nc(-c3ccc4c(c3)c3ccccc3n4-c3ccc4oc5c(-c6ccccc6)cccc5c4c3)nc(-c3cccc4oc5ccccc5c34)n2)cc1] |
| 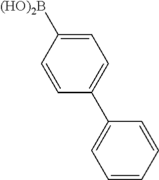 [SMILES: OB(O)c1ccc(-c2ccccc2)cc1] | 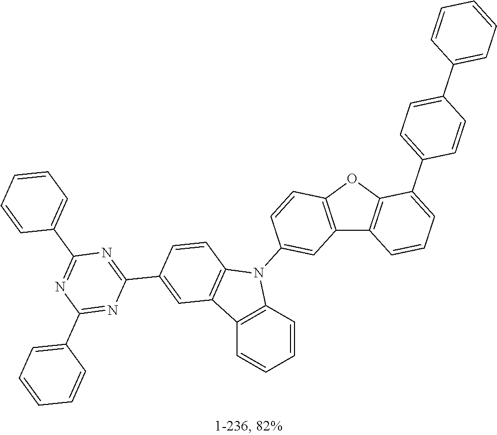 [SMILES: c1ccc(-c2ccc(-c3cccc4c3oc3ccc(-n5c6ccccc6c6cc(-c7nc(-c8ccccc8)nc(-c8ccccc8)n7)ccc65)cc34)cc2)cc1] |
| 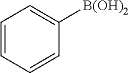 | 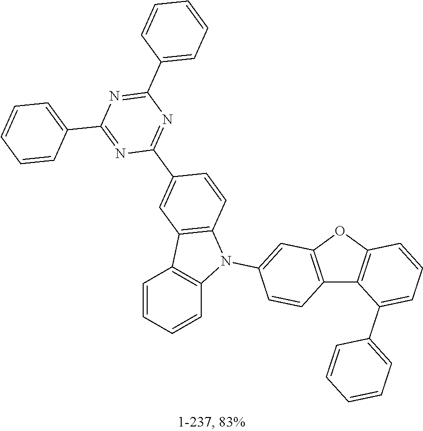 [SMILES: c1ccc(-c2nc(-c3ccccc3)nc(-c3ccc4c(c3)c3ccccc3n4-c3ccc4c(c3)oc3cccc(-c5ccccc5)c34)n2)cc1] |
| 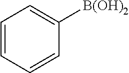 | 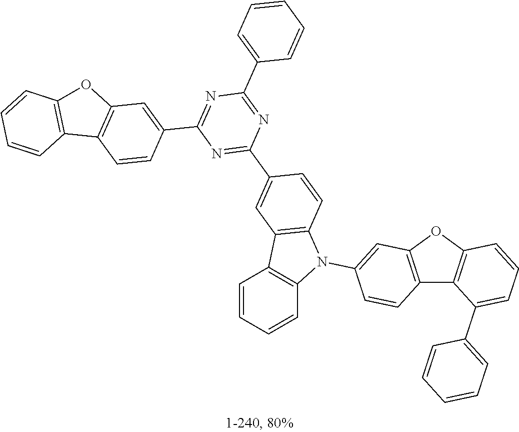 [SMILES: c1ccc(-c2nc(-c3ccc4c(c3)oc3ccccc34)nc(-c3ccc4c(c3)c3ccccc3n4-c3ccc4c(c3)oc3cccc(-c5ccccc5)c34)n2)cc1] |
| 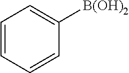 | 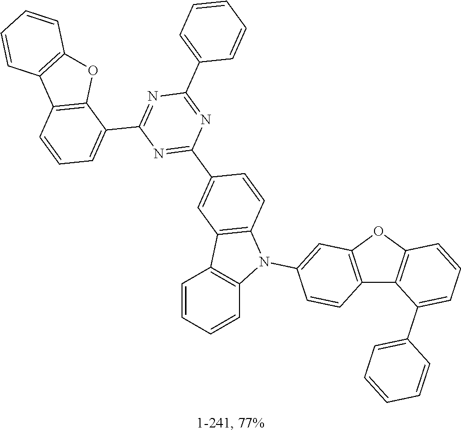 [SMILES: c1ccc(-c2nc(-c3ccc4c(c3)c3ccccc3n4-c3ccc4c(c3)oc3cccc(-c5ccccc5)c34)nc(-c3cccc4c3oc3ccccc34)n2)cc1] |
| 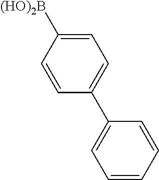 [SMILES: OB(O)c1ccc(-c2ccccc2)cc1] | 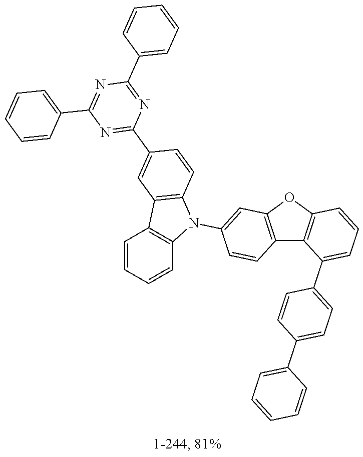 [SMILES: c1ccc(-c2ccc(-c3cccc4oc5cc(-n6c7ccccc7c7cc(-c8nc(-c9ccccc9)nc(-c9ccccc9)n8)ccc76)ccc5c34)cc2)cc1] |
| 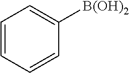 | 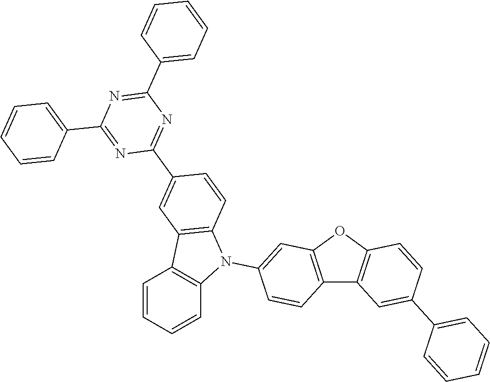 [SMILES: c1ccc(-c2ccc3oc4cc(-n5c6ccccc6c6cc(-c7nc(-c8ccccc8)nc(-c8ccccc8)n7)ccc65)ccc4c3c2)cc1] |
| | 1-245, 80% |
| 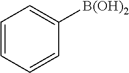 | 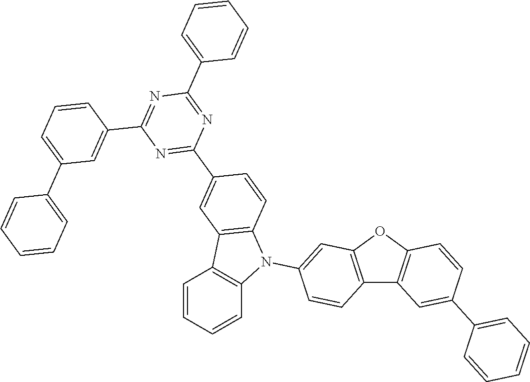 [SMILES: c1ccc(-c2cccc(-c3nc(-c4ccccc4)nc(-c4ccc5c(c4)c4ccccc4n5-c4ccc5c(c4)oc4ccc(-c6ccccc6)cc45)n3)c2)cc1] |
| | 1-247, 77% |
| 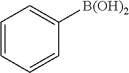 | 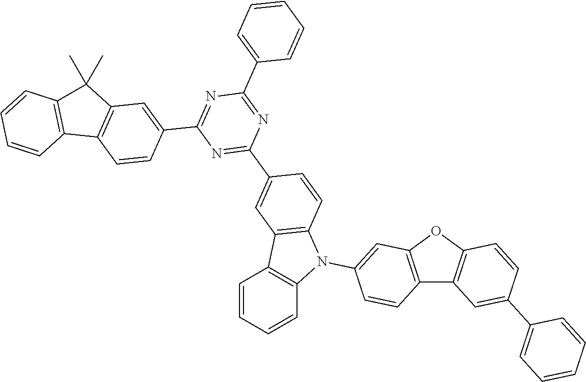 [SMILES: CC1(C)c2ccccc2-c2ccc(-c3nc(-c4ccccc4)nc(-c4ccc5c(c4)c4ccccc4n5-c4ccc5c(c4)oc4ccc(-c6ccccc6)cc45)n3)cc21] |
| | 1-251, 73% |
| 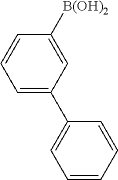 [SMILES: OB(O)c1cccc(-c2ccccc2)c1] | 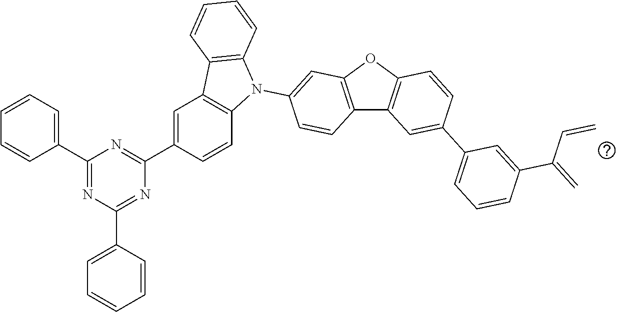 [SMILES: C=CC(=C)c1cccc(-c2ccc3oc4cc(-n5c6ccccc6c6cc(-c7nc(-c8ccccc8)nc(-c8ccccc8)n7)ccc65)ccc4c3c2)c1] |
| | 1-252, 79% |
| 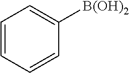 | 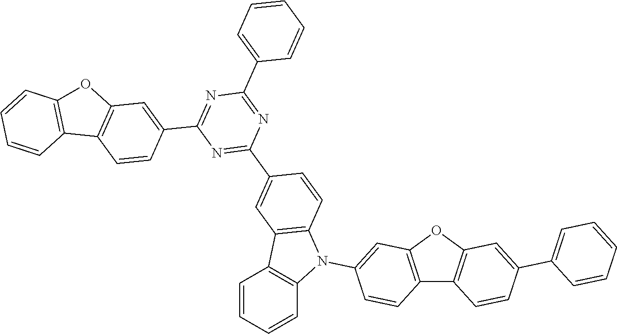 [SMILES: c1ccc(-c2ccc3c(c2)oc2cc(-n4c5ccccc5c5cc(-c6nc(-c7ccccc7)nc(-c7ccc8c(c7)oc7ccccc78)n6)ccc54)ccc23)cc1] |
| | 1-256, 77% |
| 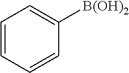 | 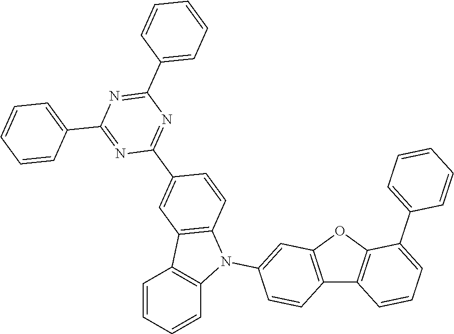 [SMILES: c1ccc(-c2nc(-c3ccccc3)nc(-c3ccc4c(c3)c3ccccc3n4-c3ccc4c(c3)oc3c(-c5ccccc5)cccc34)n2)cc1] |
| | 1-261, 80% |
| 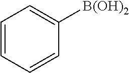 | 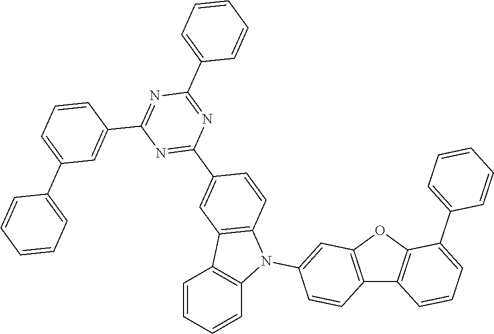 [SMILES: c1ccc(-c2cccc(-c3nc(-c4ccccc4)nc(-c4ccc5c(c4)c4ccccc4n5-c4ccc5c(c4)oc4c(-c6ccccc6)cccc45)n3)c2)cc1] |
| | 1-263, 74% |
| 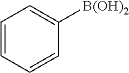 | 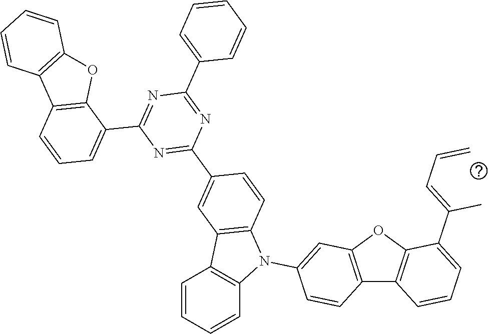 [SMILES: C=C/C=C(\C)c1cccc2c1oc1cc(-n3c4ccccc4c4cc(-c5nc(-c6ccccc6)nc(-c6cccc7c6oc6ccccc67)n5)ccc43)ccc12] |
| | 1-265, 74% |
| 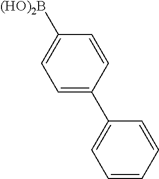 [SMILES: OB(O)c1ccc(-c2ccccc2)cc1] | 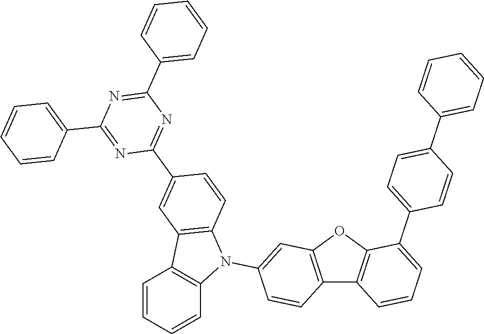 [SMILES: c1ccc(-c2ccc(-c3cccc4c3oc3cc(-n5c6ccccc6c6cc(-c7nc(-c8ccccc8)nc(-c8ccccc8)n7)ccc65)ccc34)cc2)cc1] |
| | 1-268, 81% |
| 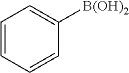 | 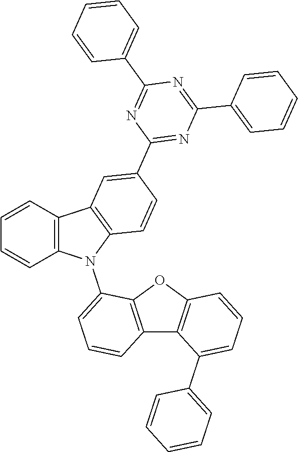 [SMILES: c1ccc(-c2nc(-c3ccccc3)nc(-c3ccc4c(c3)c3ccccc3n4-c3cccc4c3oc3cccc(-c5ccccc5)c34)n2)cc1] |
| | 1-269, 83% |
| 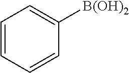 | 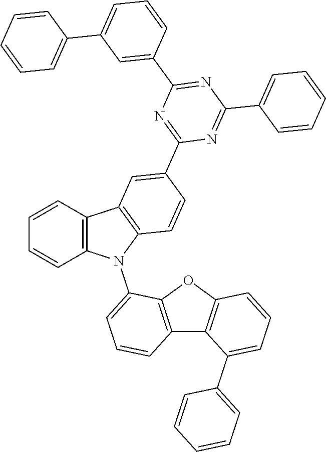 [SMILES: c1ccc(-c2cccc(-c3nc(-c4ccccc4)nc(-c4ccc5c(c4)c4ccccc4n5-c4cccc5c4oc4cccc(-c6ccccc6)c45)n3)c2)cc1] |
| | 1-271, 80% |
| 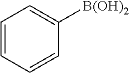 | 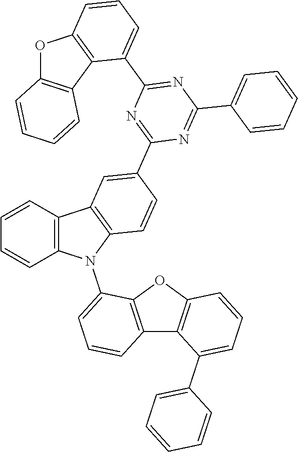 [SMILES: c1ccc(-c2nc(-c3ccc4c(c3)c3ccccc3n4-c3cccc4c3oc3cccc(-c5ccccc5)c34)nc(-c3cccc4oc5ccccc5c34)n2)cc1] |
| | 1-274, 76% |
| 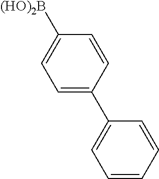 [SMILES: OB(O)c1ccc(-c2ccccc2)cc1] | 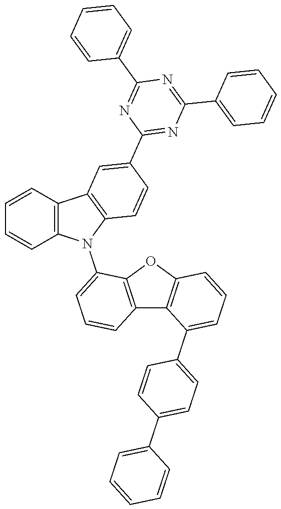 [SMILES: c1ccc(-c2ccc(-c3cccc4oc5c(-n6c7ccccc7c7cc(-c8nc(-c9ccccc9)nc(-c9ccccc9)n8)ccc76)cccc5c34)cc2)cc1] |
| | 1-276, 77% |
| 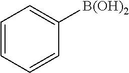 | 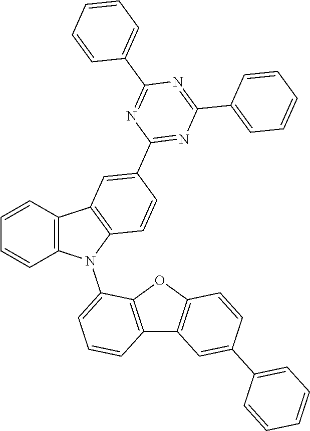 [SMILES: c1ccc(-c2ccc3oc4c(-n5c6ccccc6c6cc(-c7nc(-c8ccccc8)nc(-c8ccccc8)n7)ccc65)cccc4c3c2)cc1] |
| | 1-277, 82% |
| 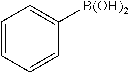 | 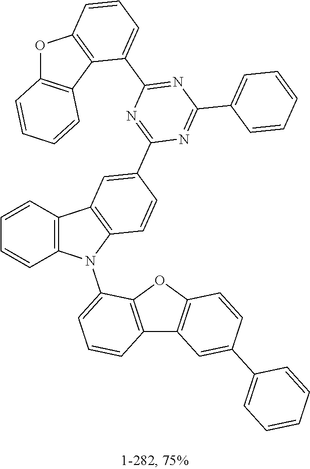 [SMILES: c1ccc(-c2ccc3oc4c(-n5c6ccccc6c6cc(-c7nc(-c8ccccc8)nc(-c8cccc9oc%10ccccc%10c89)n7)ccc65)cccc4c3c2)cc1] |
| 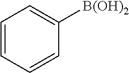 | 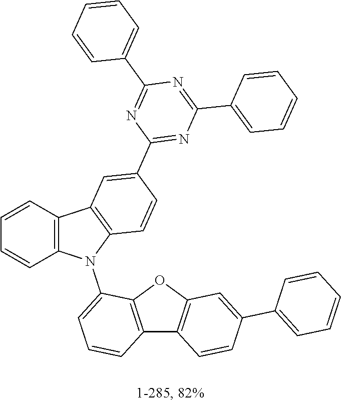 [SMILES: c1ccc(-c2ccc3c(c2)oc2c(-n4c5ccccc5c5cc(-c6nc(-c7ccccc7)nc(-c7ccccc7)n6)ccc54)cccc23)cc1] |
| 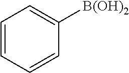 | 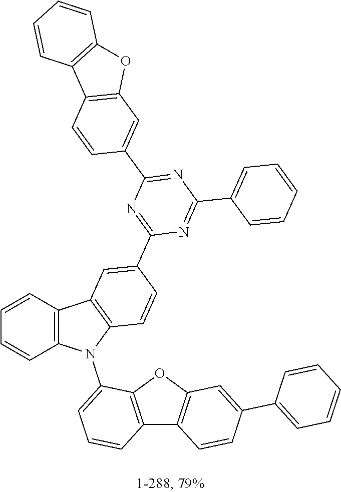 [SMILES: c1ccc(-c2ccc3c(c2)oc2c(-n4c5ccccc5c5cc(-c6nc(-c7ccccc7)nc(-c7ccc8c(c7)oc7ccccc78)n6)ccc54)cccc23)cc1] |
| 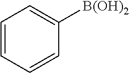 | 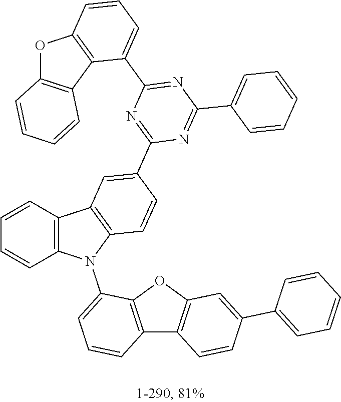 [SMILES: c1ccc(-c2ccc3c(c2)oc2c(-n4c5ccccc5c5cc(-c6nc(-c7ccccc7)nc(-c7cccc8oc9ccccc9c78)n6)ccc54)cccc23)cc1] |
| 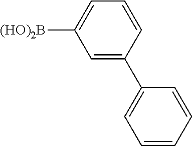 [SMILES: OB(O)c1cccc(-c2ccccc2)c1] | 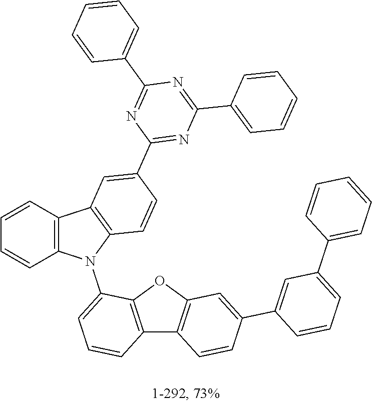 [SMILES: c1ccc(-c2cccc(-c3ccc4c(c3)oc3c(-n5c6ccccc6c6cc(-c7nc(-c8ccccc8)nc(-c8ccccc8)n7)ccc65)cccc34)c2)cc1] |
| 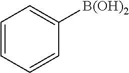 | 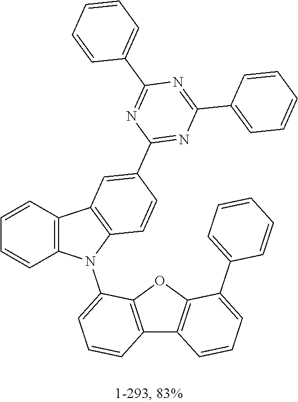 [SMILES: c1ccc(-c2nc(-c3ccccc3)nc(-c3ccc4c(c3)c3ccccc3n4-c3cccc4c3oc3c(-c5ccccc5)cccc34)n2)cc1] |
| 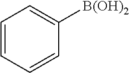 | 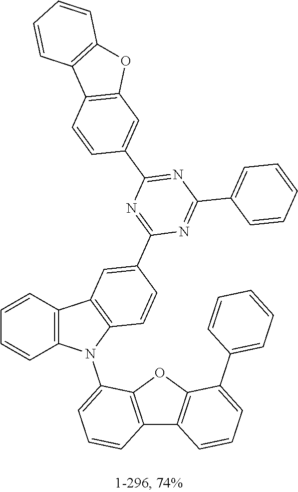 [SMILES: c1ccc(-c2nc(-c3ccc4c(c3)oc3ccccc34)nc(-c3ccc4c(c3)c3ccccc3n4-c3cccc4c3oc3c(-c5ccccc5)cccc34)n2)cc1] |
| 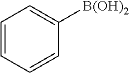 | 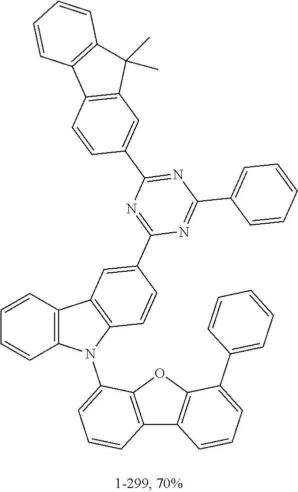 [SMILES: CC1(C)c2ccccc2-c2ccc(-c3nc(-c4ccccc4)nc(-c4ccc5c(c4)c4ccccc4n5-c4cccc5c4oc4c(-c6ccccc6)cccc45)n3)cc21] |
| 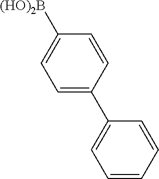 [SMILES: OB(O)c1ccc(-c2ccccc2)cc1] | 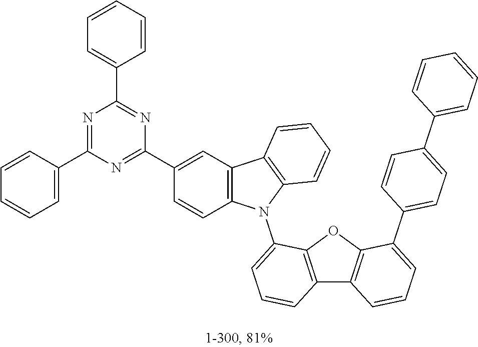 [SMILES: c1ccc(-c2ccc(-c3cccc4c3oc3c(-n5c6ccccc6c6cc(-c7nc(-c8ccccc8)nc(-c8ccccc8)n7)ccc65)cccc34)cc2)cc1] |
| 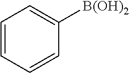 | 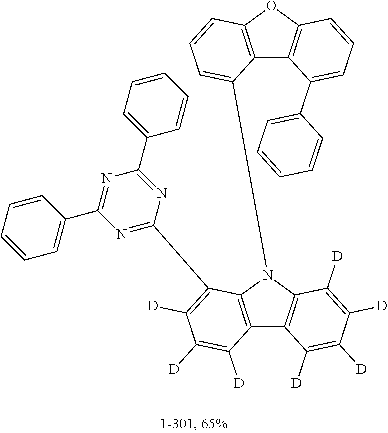 [SMILES: [2H]c1c([2H])c([2H])c2c(c1[2H])c1c([2H])c([2H])c([2H])c(-c3nc(-c4ccccc4)nc(-c4ccccc4)n3)c1n2-c1cccc2oc3cccc(-c4ccccc4)c3c12] |
| 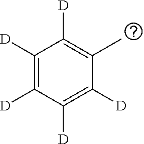 | 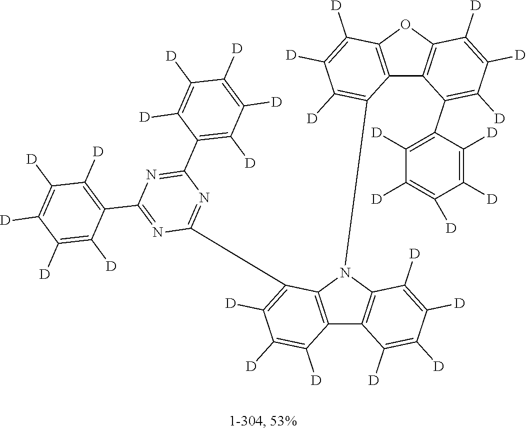 [SMILES: [2H]c1c([2H])c([2H])c(-c2nc(-c3c([2H])c([2H])c([2H])c([2H])c3[2H])nc(-c3c([2H])c([2H])c([2H])c4c5c([2H])c([2H])c([2H])c([2H])c5n(-c5c([2H])c([2H])c([2H])c6oc7c([2H])c([2H])c([2H])c(-c8c([2H])c([2H])c([2H])c([2H])c8[2H])c7c56)c34)n2)c([2H])c1[2H]] |
| 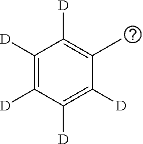 | 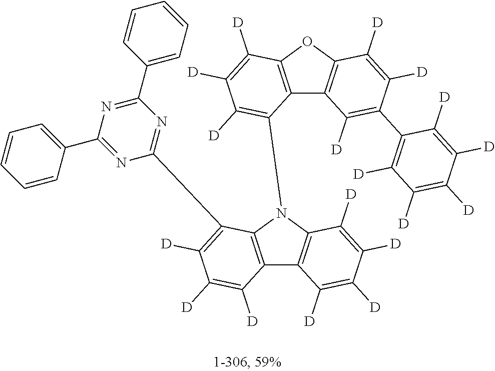 [SMILES: [2H]c1c([2H])c([2H])c(-c2c([2H])c([2H])c3oc4c([2H])c([2H])c([2H])c(-n5c6c([2H])c([2H])c([2H])c([2H])c6c6c([2H])c([2H])c([2H])c(-c7nc(-c8ccccc8)nc(-c8ccccc8)n7)c65)c4c3c2[2H])c([2H])c1[2H]] |
| 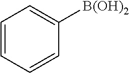 | 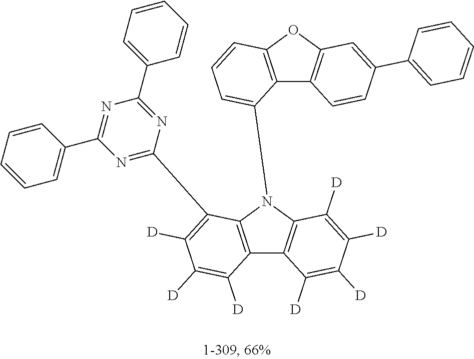 [SMILES: [2H]c1c([2H])c([2H])c2c(c1[2H])c1c([2H])c([2H])c([2H])c(-c3nc(-c4ccccc4)nc(-c4ccccc4)n3)c1n2-c1cccc2oc3cc(-c4ccccc4)ccc3c12] |
| 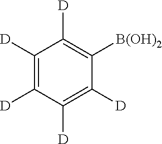 [SMILES: [2H]c1c([2H])c([2H])c(B(O)O)c([2H])c1[2H]] | 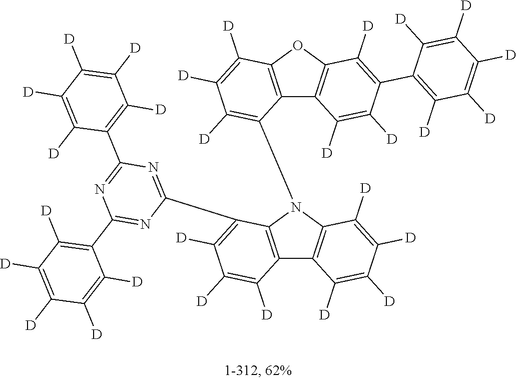 [SMILES: [2H]c1c([2H])c([2H])c(-c2nc(-c3c([2H])c([2H])c([2H])c([2H])c3[2H])nc(-c3c([2H])c([2H])c([2H])c4c5c([2H])c([2H])c([2H])c([2H])c5n(-c5c([2H])c([2H])c([2H])c6oc7c([2H])c(-c8c([2H])c([2H])c([2H])c([2H])c8[2H])c([2H])c([2H])c7c56)c34)n2)c([2H])c1[2H]] |
| 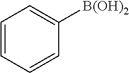 | 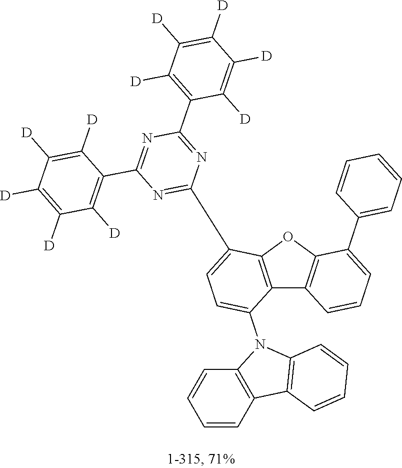 [SMILES: [2H]c1c([2H])c([2H])c(-c2nc(-c3c([2H])c([2H])c([2H])c([2H])c3[2H])nc(-c3ccc(-n4c5ccccc5c5ccccc54)c4c3oc3c(-c5ccccc5)cccc34)n2)c([2H])c1[2H]] |
| 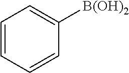 | 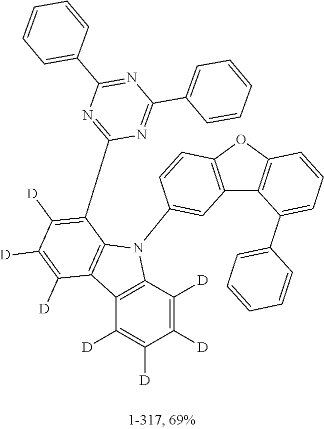 [SMILES: [2H]c1c([2H])c([2H])c2c(c1[2H])c1c([2H])c([2H])c([2H])c(-c3nc(-c4ccccc4)nc(-c4ccccc4)n3)c1n2-c1ccc2oc3cccc(-c4ccccc4)c3c2c1] |
| 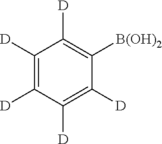 [SMILES: [2H]c1c([2H])c([2H])c(B(O)O)c([2H])c1[2H]] | 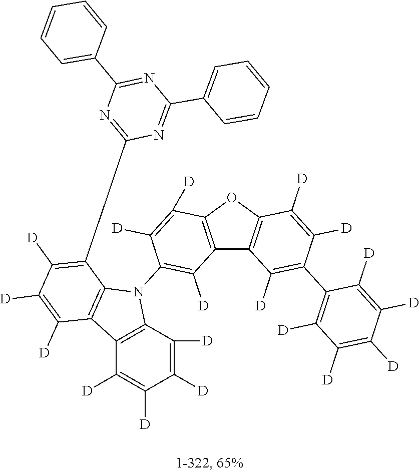 [SMILES: [2H]c1c([2H])c([2H])c(-c2c([2H])c([2H])c3oc4c([2H])c([2H])c(-n5c6c([2H])c([2H])c([2H])c([2H])c6c6c([2H])c([2H])c([2H])c(-c7nc(-c8ccccc8)nc(-c8ccccc8)n7)c65)c([2H])c4c3c2[2H])c([2H])c1[2H]] |
| 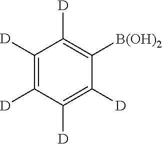 [SMILES: [2H]c1c([2H])c([2H])c(B(O)O)c([2H])c1[2H]] | 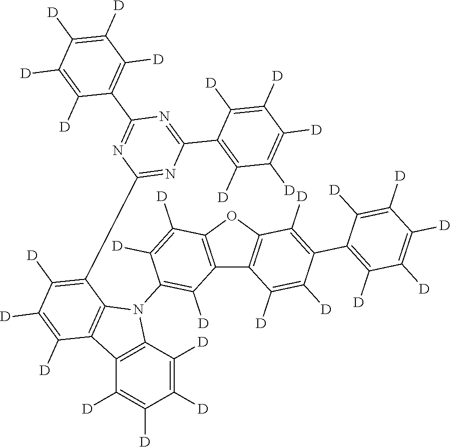 [SMILES: [2H]c1c([2H])c([2H])c(-c2nc(-c3c([2H])c([2H])c([2H])c([2H])c3[2H])nc(-c3c([2H])c([2H])c([2H])c4c5c([2H])c([2H])c([2H])c([2H])c5n(-c5c([2H])c([2H])c6oc7c([2H])c(-c8c([2H])c([2H])c([2H])c([2H])c8[2H])c([2H])c([2H])c7c6c5[2H])c34)n2)c([2H])c1[2H]] |
| | 1-328, 60% |
| 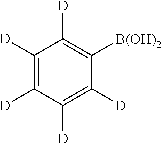 [SMILES: [2H]c1c([2H])c([2H])c(B(O)O)c([2H])c1[2H]] | 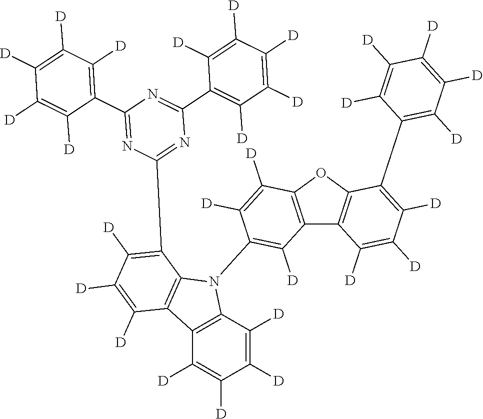 [SMILES: [2H]c1c([2H])c([2H])c(-c2nc(-c3c([2H])c([2H])c([2H])c([2H])c3[2H])nc(-c3c([2H])c([2H])c([2H])c4c5c([2H])c([2H])c([2H])c([2H])c5n(-c5c([2H])c([2H])c6oc7c(-c8c([2H])c([2H])c([2H])c([2H])c8[2H])c([2H])c([2H])c([2H])c7c6c5[2H])c34)n2)c([2H])c1[2H]] |
| | 1-332, 59% |
| 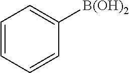 | 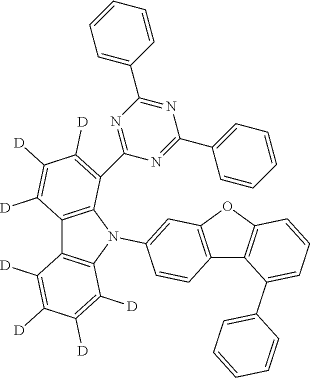 [SMILES: [2H]c1c([2H])c([2H])c2c(c1[2H])c1c([2H])c([2H])c([2H])c(-c3nc(-c4ccccc4)nc(-c4ccccc4)n3)c1n2-c1ccc2c(c1)oc1cccc(-c3ccccc3)c12] |
| | 1-333, 68% |
| 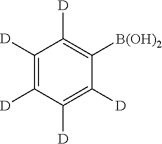 [SMILES: [2H]c1c([2H])c([2H])c(B(O)O)c([2H])c1[2H]] | 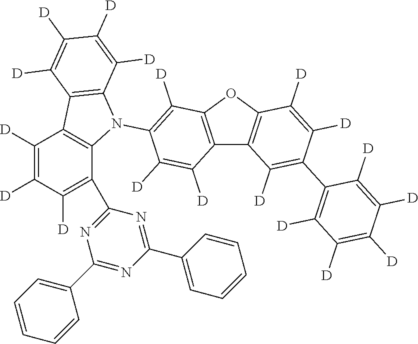 [SMILES: [2H]c1c([2H])c([2H])c(-c2c([2H])c([2H])c3oc4c([2H])c(-n5c6c([2H])c([2H])c([2H])c([2H])c6c6c([2H])c([2H])c([2H])c(-c7nc(-c8ccccc8)nc(-c8ccccc8)n7)c65)c([2H])c([2H])c4c3c2[2H])c([2H])c1[2H]] |
| | 1-338, 62% |
| 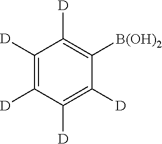 [SMILES: [2H]c1c([2H])c([2H])c(B(O)O)c([2H])c1[2H]] | 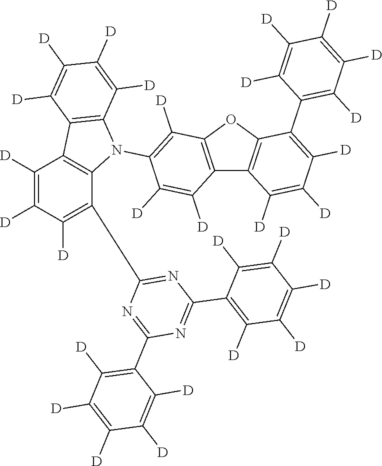 [SMILES: [2H]c1c([2H])c([2H])c(-c2nc(-c3c([2H])c([2H])c([2H])c([2H])c3[2H])nc(-c3c([2H])c([2H])c([2H])c4c5c([2H])c([2H])c([2H])c([2H])c5n(-c5c([2H])c([2H])c6c(oc7c(-c8c([2H])c([2H])c([2H])c([2H])c8[2H])c([2H])c([2H])c([2H])c76)c5[2H])c34)n2)c([2H])c1[2H]] |
| | 1-344, 60% |
| 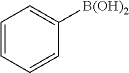 | 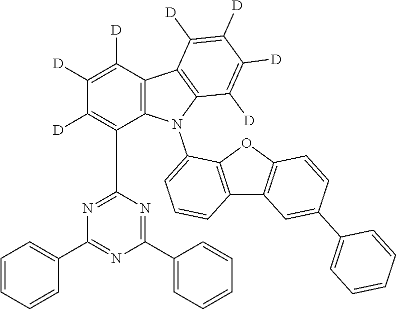 [SMILES: [2H]c1c([2H])c([2H])c2c(c1[2H])c1c([2H])c([2H])c([2H])c(-c3nc(-c4ccccc4)nc(-c4ccccc4)n3)c1n2-c1cccc2c1oc1ccc(-c3ccccc3)cc12] |
| | 1-349, 66% |
| 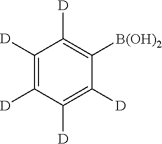 [SMILES: [2H]c1c([2H])c([2H])c(B(O)O)c([2H])c1[2H]] | 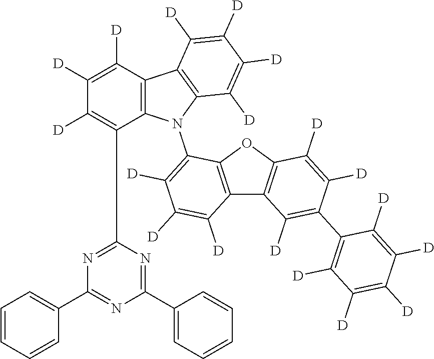 [SMILES: [2H]c1c([2H])c([2H])c(-c2c([2H])c([2H])c3oc4c(-n5c6c([2H])c([2H])c([2H])c([2H])c6c6c([2H])c([2H])c([2H])c(-c7nc(-c8ccccc8)nc(-c8ccccc8)n7)c65)c([2H])c([2H])c([2H])c4c3c2[2H])c([2H])c1[2H]] |
| | 1-350, 64% |
| 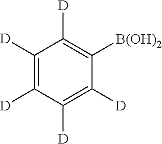 [SMILES: [2H]c1c([2H])c([2H])c(B(O)O)c([2H])c1[2H]] | 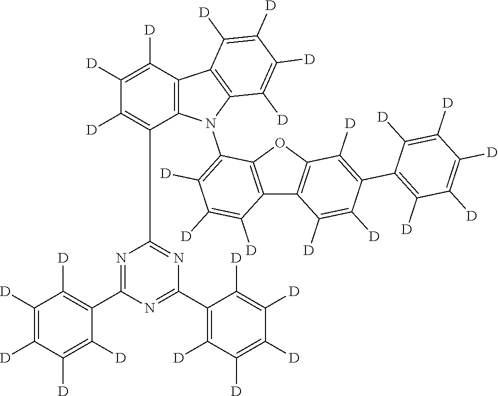 [SMILES: [2H]c1c([2H])c([2H])c(-c2nc(-c3c([2H])c([2H])c([2H])c([2H])c3[2H])nc(-c3c([2H])c([2H])c([2H])c4c5c([2H])c([2H])c([2H])c([2H])c5n(-c5c([2H])c([2H])c([2H])c6c5oc5c([2H])c(-c7c([2H])c([2H])c([2H])c([2H])c7[2H])c([2H])c([2H])c56)c34)n2)c([2H])c1[2H]] |
| | 1-356, 58% |
| 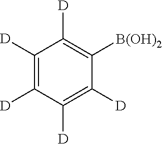 [SMILES: [2H]c1c([2H])c([2H])c(B(O)O)c([2H])c1[2H]] | 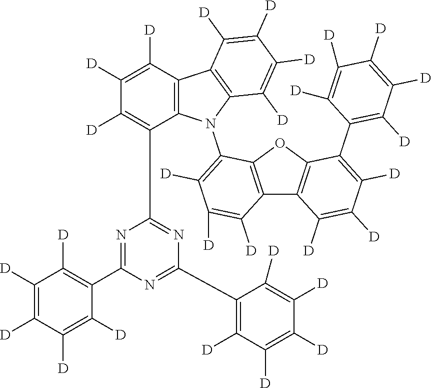 [SMILES: [2H]c1c([2H])c([2H])c(-c2nc(-c3c([2H])c([2H])c([2H])c([2H])c3[2H])nc(-c3c([2H])c([2H])c([2H])c4c5c([2H])c([2H])c([2H])c([2H])c5n(-c5c([2H])c([2H])c([2H])c6c5oc5c(-c7c([2H])c([2H])c([2H])c([2H])c7[2H])c([2H])c([2H])c([2H])c56)c34)n2)c([2H])c1[2H]] |
| | 1-360, 58% |
| 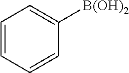 | 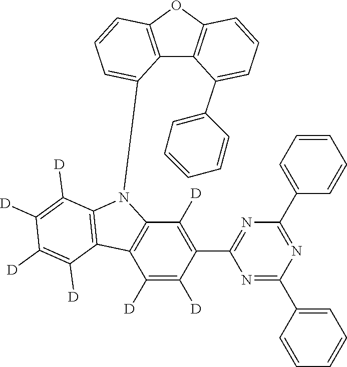 [SMILES: [2H]c1c([2H])c([2H])c2c(c1[2H])c1c([2H])c([2H])c(-c3nc(-c4ccccc4)nc(-c4ccccc4)n3)c([2H])c1n2-c1cccc2oc3cccc(-c4ccccc4)c3c12] |
| | 1-361, 69% |
| 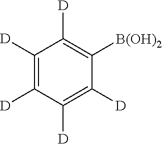 [SMILES: [2H]c1c([2H])c([2H])c(B(O)O)c([2H])c1[2H]] | 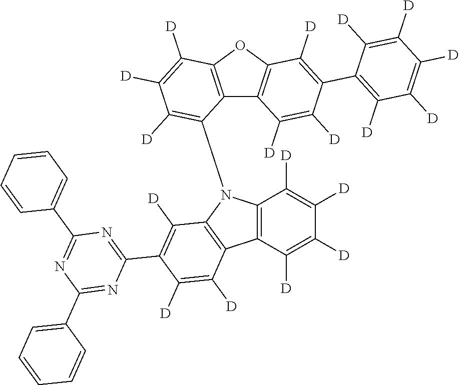 [SMILES: [2H]c1c([2H])c([2H])c(-c2c([2H])c([2H])c3c(oc4c([2H])c([2H])c([2H])c(-n5c6c([2H])c([2H])c([2H])c([2H])c6c6c([2H])c([2H])c(-c7nc(-c8ccccc8)nc(-c8ccccc8)n7)c([2H])c65)c43)c2[2H])c([2H])c1[2H]] |
| | 1-366, 61% |
| 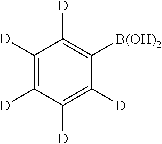 [SMILES: [2H]c1c([2H])c([2H])c(B(O)O)c([2H])c1[2H]] | 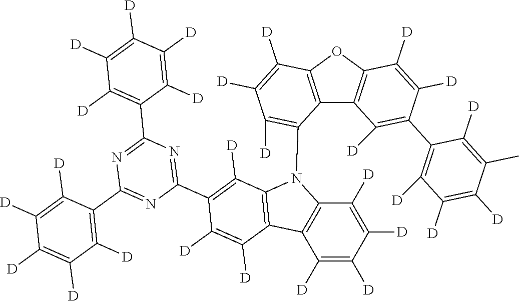 [SMILES: [2H]c1c([2H])c([2H])c(-c2nc(-c3c([2H])c([2H])c([2H])c([2H])c3[2H])nc(-c3c([2H])c([2H])c4c5c([2H])c([2H])c([2H])c([2H])c5n(-c5c([2H])c([2H])c([2H])c6oc7c([2H])c([2H])c(-c8c([2H])c([2H])c([2H])c(C)c8[2H])c([2H])c7c56)c4c3[2H])n2)c([2H])c1[2H]] |
| | 1-372, 55% |
| 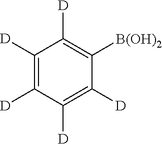 [SMILES: [2H]c1c([2H])c([2H])c(B(O)O)c([2H])c1[2H]] | 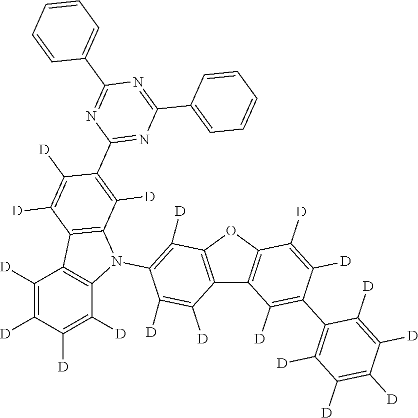 [SMILES: [2H]c1c([2H])c([2H])c(-c2c([2H])c([2H])c3oc4c([2H])c(-n5c6c([2H])c([2H])c([2H])c([2H])c6c6c([2H])c([2H])c(-c7nc(-c8ccccc8)nc(-c8ccccc8)n7)c([2H])c65)c([2H])c([2H])c4c3c2[2H])c([2H])c1[2H]] |
| | 1-374, 63% |
| 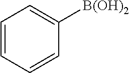 | 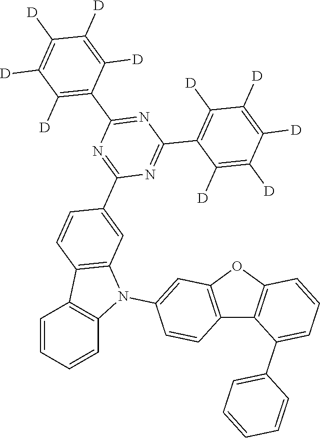 [SMILES: [2H]c1c([2H])c([2H])c(-c2nc(-c3ccc4c5ccccc5n(-c5ccc6c(c5)oc5cccc(-c7ccccc7)c56)c4c3)nc(-c3c([2H])c([2H])c([2H])c([2H])c3[2H])n2)c([2H])c1[2H]] |
| | 1-379, 66% |
| 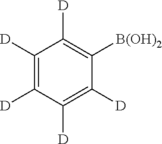 [SMILES: [2H]c1c([2H])c([2H])c(B(O)O)c([2H])c1[2H]] | 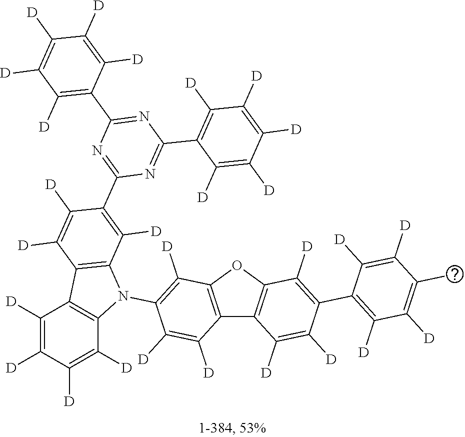 [SMILES: [2H]c1c([2H])c([2H])c(-c2nc(-c3c([2H])c([2H])c([2H])c([2H])c3[2H])nc(-c3c([2H])c([2H])c4c5c([2H])c([2H])c([2H])c([2H])c5n(-c5c([2H])c([2H])c6c(oc7c([2H])c(-c8c([2H])c([2H])c(C)c([2H])c8[2H])c([2H])c([2H])c76)c5[2H])c4c3[2H])n2)c([2H])c1[2H]] |
| 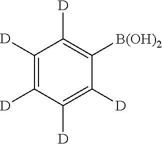 [SMILES: [2H]c1c([2H])c([2H])c(B(O)O)c([2H])c1[2H]] | 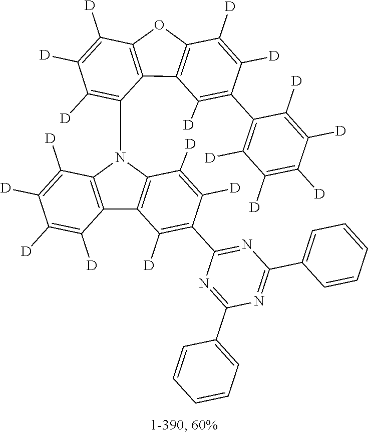 [SMILES: [2H]c1c([2H])c([2H])c(-c2c([2H])c([2H])c3oc4c([2H])c([2H])c([2H])c(-n5c6c([2H])c([2H])c([2H])c([2H])c6c6c([2H])c(-c7nc(-c8ccccc8)nc(-c8ccccc8)n7)c([2H])c([2H])c65)c4c3c2[2H])c([2H])c1[2H]] |
| 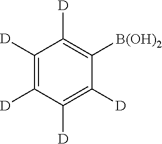 [SMILES: [2H]c1c([2H])c([2H])c(B(O)O)c([2H])c1[2H]] | 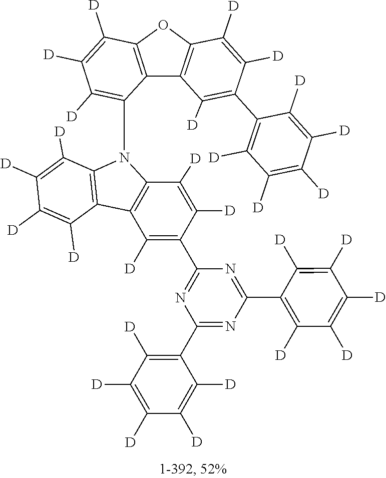 [SMILES: [2H]c1c([2H])c([2H])c(-c2nc(-c3c([2H])c([2H])c([2H])c([2H])c3[2H])nc(-c3c([2H])c([2H])c4c(c3[2H])c3c([2H])c([2H])c([2H])c([2H])c3n4-c3c([2H])c([2H])c([2H])c4oc5c([2H])c([2H])c(-c6c([2H])c([2H])c([2H])c([2H])c6[2H])c([2H])c5c34)n2)c([2H])c1[2H]] |
| 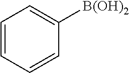 | 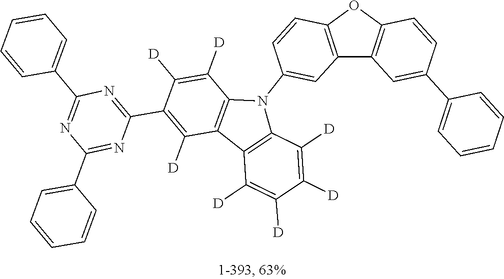 [SMILES: [2H]c1c([2H])c([2H])c2c(c1[2H])c1c([2H])c(-c3nc(-c4ccccc4)nc(-c4ccccc4)n3)c([2H])c([2H])c1n2-c1ccc2oc3ccc(-c4ccccc4)cc3c2c1] |
| 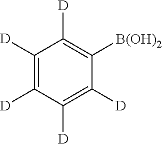 [SMILES: [2H]c1c([2H])c([2H])c(B(O)O)c([2H])c1[2H]] | 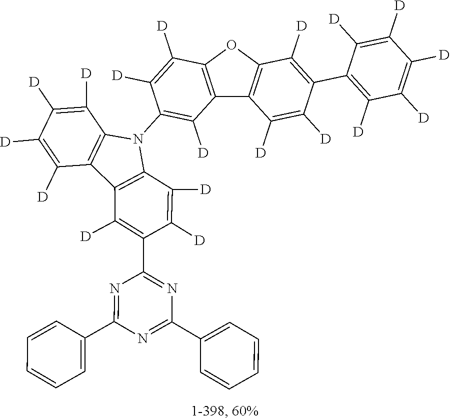 [SMILES: [2H]c1c([2H])c([2H])c(-c2c([2H])c([2H])c3c(oc4c([2H])c([2H])c(-n5c6c([2H])c([2H])c([2H])c([2H])c6c6c([2H])c(-c7nc(-c8ccccc8)nc(-c8ccccc8)n7)c([2H])c([2H])c65)c([2H])c43)c2[2H])c([2H])c1[2H]] |
| 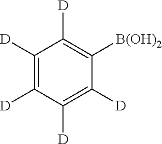 [SMILES: [2H]c1c([2H])c([2H])c(B(O)O)c([2H])c1[2H]] | 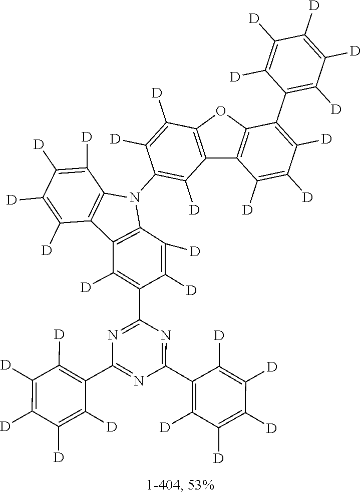 [SMILES: [2H]c1c([2H])c([2H])c(-c2nc(-c3c([2H])c([2H])c([2H])c([2H])c3[2H])nc(-c3c([2H])c([2H])c4c(c3[2H])c3c([2H])c([2H])c([2H])c([2H])c3n4-c3c([2H])c([2H])c4oc5c(-c6c([2H])c([2H])c([2H])c([2H])c6[2H])c([2H])c([2H])c([2H])c5c4c3[2H])n2)c([2H])c1[2H]] |
| 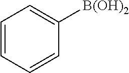 | 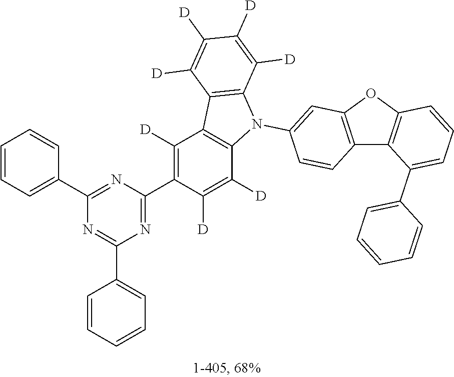 [SMILES: [2H]c1c([2H])c([2H])c2c(c1[2H])c1c([2H])c(-c3nc(-c4ccccc4)nc(-c4ccccc4)n3)c([2H])c([2H])c1n2-c1ccc2c(c1)oc1cccc(-c3ccccc3)c12] |
| 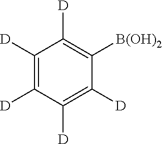 [SMILES: [2H]c1c([2H])c([2H])c(B(O)O)c([2H])c1[2H]] | 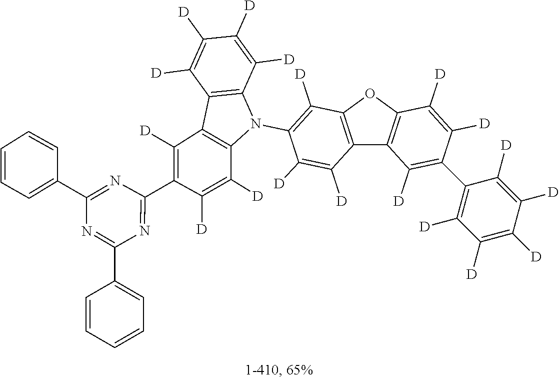 [SMILES: [2H]c1c([2H])c([2H])c(-c2c([2H])c([2H])c3oc4c([2H])c(-n5c6c([2H])c([2H])c([2H])c([2H])c6c6c([2H])c(-c7nc(-c8ccccc8)nc(-c8ccccc8)n7)c([2H])c([2H])c65)c([2H])c([2H])c4c3c2[2H])c([2H])c1[2H]] |
| 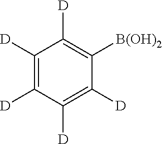 [SMILES: [2H]c1c([2H])c([2H])c(B(O)O)c([2H])c1[2H]] | 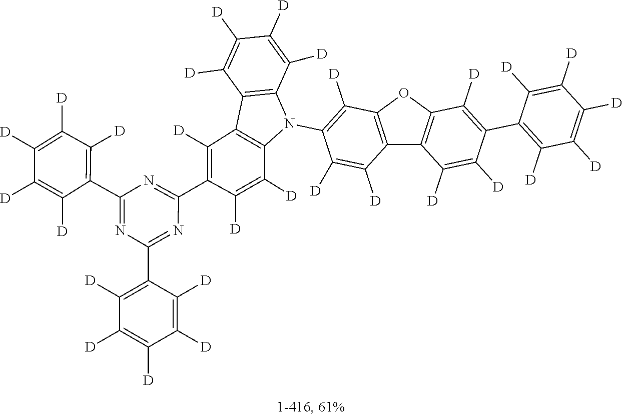 [SMILES: [2H]c1c([2H])c([2H])c(-c2nc(-c3c([2H])c([2H])c([2H])c([2H])c3[2H])nc(-c3c([2H])c([2H])c4c(c3[2H])c3c([2H])c([2H])c([2H])c([2H])c3n4-c3c([2H])c([2H])c4c(oc5c([2H])c(-c6c([2H])c([2H])c([2H])c([2H])c6[2H])c([2H])c([2H])c54)c3[2H])n2)c([2H])c1[2H]] |
| 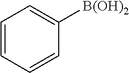 | 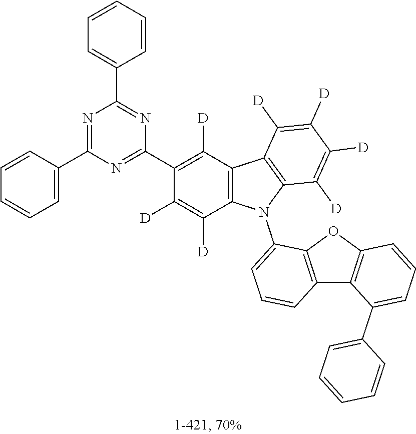 [SMILES: [2H]c1c([2H])c([2H])c2c(c1[2H])c1c([2H])c(-c3nc(-c4ccccc4)nc(-c4ccccc4)n3)c([2H])c([2H])c1n2-c1cccc2c1oc1cccc(-c3ccccc3)c12] |
| 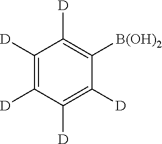 [SMILES: [2H]c1c([2H])c([2H])c(B(O)O)c([2H])c1[2H]] | 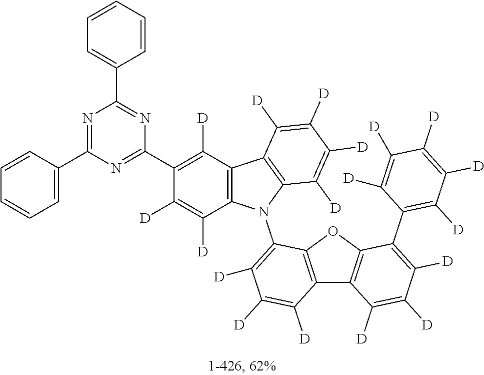 [SMILES: [2H]c1c([2H])c([2H])c(-c2c([2H])c([2H])c([2H])c3c2oc2c(-n4c5c([2H])c([2H])c([2H])c([2H])c5c5c([2H])c(-c6nc(-c7ccccc7)nc(-c7ccccc7)n6)c([2H])c([2H])c54)c([2H])c([2H])c([2H])c23)c([2H])c1[2H]] |
| 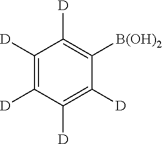 [SMILES: [2H]c1c([2H])c([2H])c(B(O)O)c([2H])c1[2H]] | 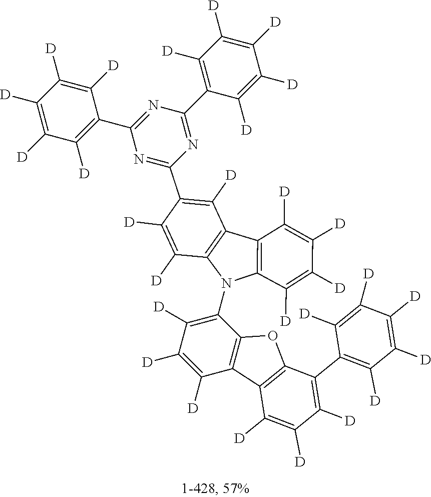 [SMILES: [2H]c1c([2H])c([2H])c(-c2nc(-c3c([2H])c([2H])c([2H])c([2H])c3[2H])nc(-c3c([2H])c([2H])c4c(c3[2H])c3c([2H])c([2H])c([2H])c([2H])c3n4-c3c([2H])c([2H])c([2H])c4c3oc3c(-c5c([2H])c([2H])c([2H])c([2H])c5[2H])c([2H])c([2H])c([2H])c34)n2)c([2H])c1[2H]] |
| 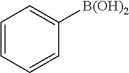 | 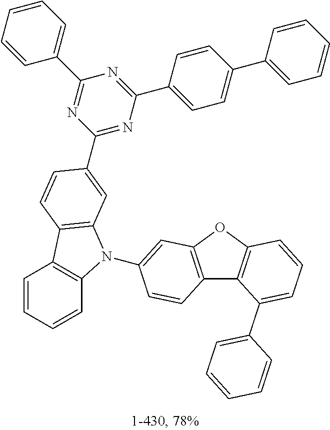 [SMILES: c1ccc(-c2ccc(-c3nc(-c4ccccc4)nc(-c4ccc5c6ccccc6n(-c6ccc7c(c6)oc6cccc(-c8ccccc8)c67)c5c4)n3)cc2)cc1] |
| 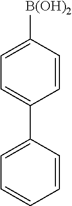 [SMILES: OB(O)c1ccc(-c2ccccc2)cc1] | 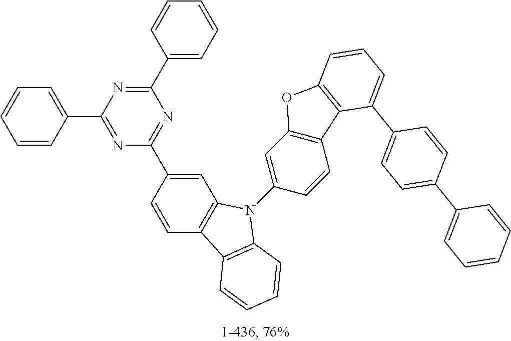 [SMILES: c1ccc(-c2ccc(-c3cccc4oc5cc(-n6c7ccccc7c7ccc(-c8nc(-c9ccccc9)nc(-c9ccccc9)n8)cc76)ccc5c34)cc2)cc1] |
| 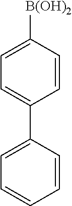 [SMILES: OB(O)c1ccc(-c2ccccc2)cc1] | 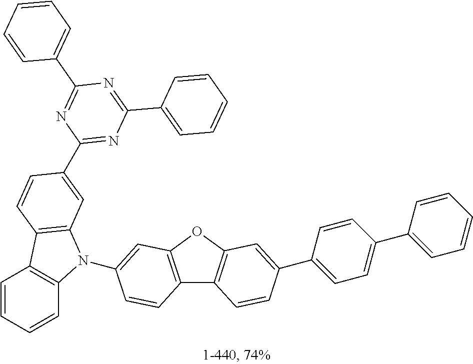 [SMILES: c1ccc(-c2ccc(-c3ccc4c(c3)oc3cc(-n5c6ccccc6c6ccc(-c7nc(-c8ccccc8)nc(-c8ccccc8)n7)cc65)ccc34)cc2)cc1] |
| 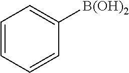 | 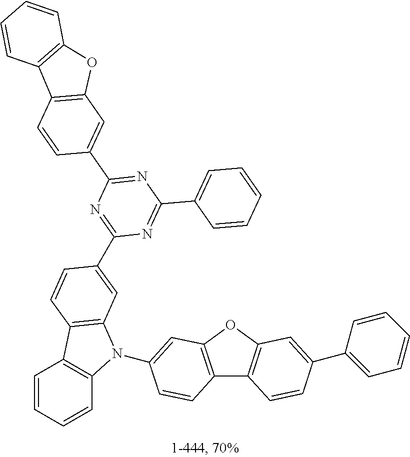 [SMILES: c1ccc(-c2ccc3c(c2)oc2cc(-n4c5ccccc5c5ccc(-c6nc(-c7ccccc7)nc(-c7ccc8c(c7)oc7ccccc78)n6)cc54)ccc23)cc1] |

### Preparation Example 2. Preparation of Compound 2-3

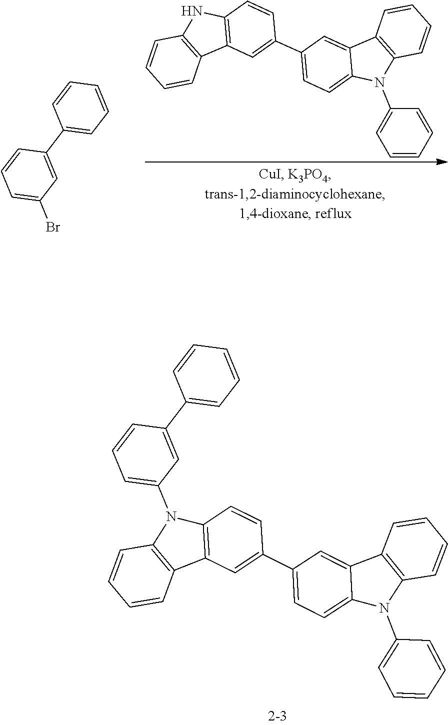

3-bromo-1,1′-biphenyl (3.7 g, 15.8 mM), 9-phenyl-9H, 9′H-3,3′-bicarbazole (6.5 g, 15.8 mM), copper iodide (CuI) (3.0 g, 15.8 mM), trans-1,2-diaminocyclohexane (1.9 mL, 15.8 mM) and K₃PO₄ (3.3 g, 31.6 mM) were dissolved in 1, 4-dioxane (100 mL), and then the mixture was refluxed for 24 hours at 110° C.

After the reaction was completed, the result was extracted by introducing distilled water and dichloromethane (DCM) thereto at room temperature, and after drying the organic layer with MgSO₄, the solvent was removed using a rotary evaporator.

The reaction material was purified by column chromatography (dichloromethane:hexane=1:3 (volume ratio)), and recrystallized with methanol to obtain target Compound 2-3 (7.5 g, yield 85%).

Target compounds were synthesized in the same manner as in Preparation Example 2, except that Intermediate a of the following Table 2 was used instead of 3-bromo-1,1′-biphenyl, and Intermediate b of the following Table 2 was used instead of 9-phenyl-9H,9′H-3,3′-bicarbazole.

TABLE 2

| | | Target Compound |
|---|---|---|
| Intermediate a | Intermediate b | (Compound No., Yield) |
| 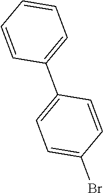 [SMILES: Brc1ccc(-c2ccccc2)cc1] | 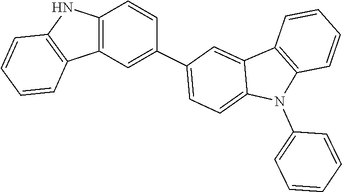 [SMILES: c1ccc(-n2c3ccccc3c3cc(-c4ccc5[nH]c6ccccc6c5c4)ccc32)cc1] | 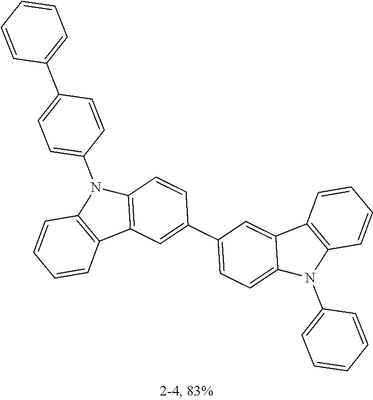 [SMILES: c1ccc(-c2ccc(-n3c4ccccc4c4cc(-c5ccc6c(c5)c5ccccc5n6-c5ccccc5)ccc43)cc2)cc1] |
| 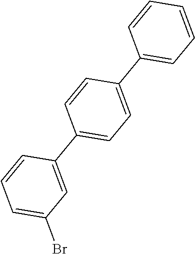 [SMILES: Brc1cccc(-c2ccc(-c3ccccc3)cc2)c1] | 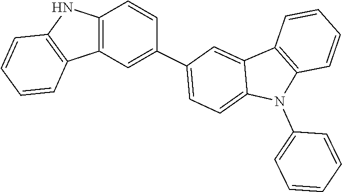 [SMILES: c1ccc(-n2c3ccccc3c3cc(-c4ccc5[nH]c6ccccc6c5c4)ccc32)cc1] | 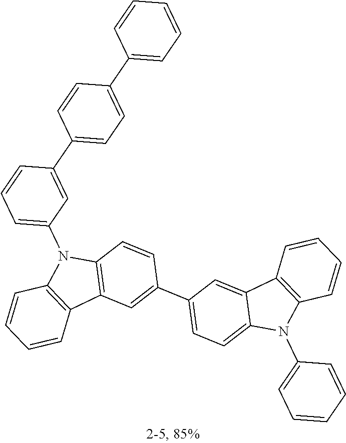 [SMILES: c1ccc(-c2ccc(-c3cccc(-n4c5ccccc5c5cc(-c6ccc7c(c6)c6ccccc6n7-c6ccccc6)ccc54)c3)cc2)cc1] |
| 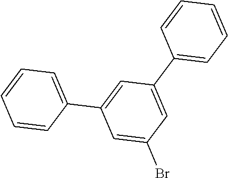 [SMILES: Brc1cc(-c2ccccc2)cc(-c2ccccc2)c1] | 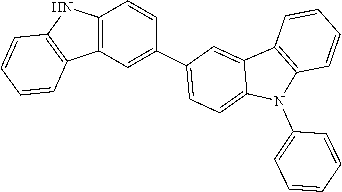 [SMILES: c1ccc(-n2c3ccccc3c3cc(-c4ccc5[nH]c6ccccc6c5c4)ccc32)cc1] | 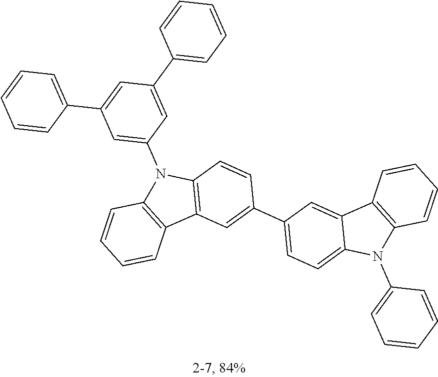 [SMILES: c1ccc(-c2cc(-c3ccccc3)cc(-n3c4ccccc4c4cc(-c5ccc6c(c5)c5ccccc5n6-c5ccccc5)ccc43)c2)cc1] |
| 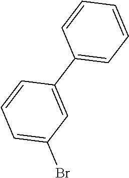 [SMILES: Brc1cccc(-c2ccccc2)c1] | 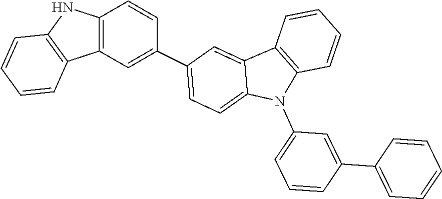 [SMILES: c1ccc(-c2cccc(-n3c4ccccc4c4cc(-c5ccc6[nH]c7ccccc7c6c5)ccc43)c2)cc1] | 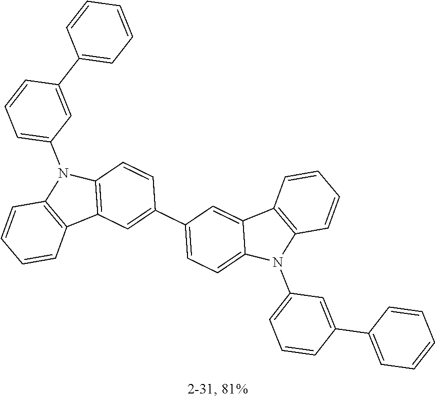 [SMILES: c1ccc(-c2cccc(-n3c4ccccc4c4cc(-c5ccc6c(c5)c5ccccc5n6-c5cccc(-c6ccccc6)c5)ccc43)c2)cc1] |
| 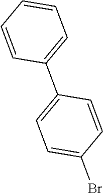 [SMILES: Brc1ccc(-c2ccccc2)cc1] | 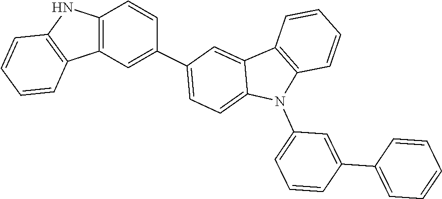 [SMILES: c1ccc(-c2cccc(-n3c4ccccc4c4cc(-c5ccc6[nH]c7ccccc7c6c5)ccc43)c2)cc1] | 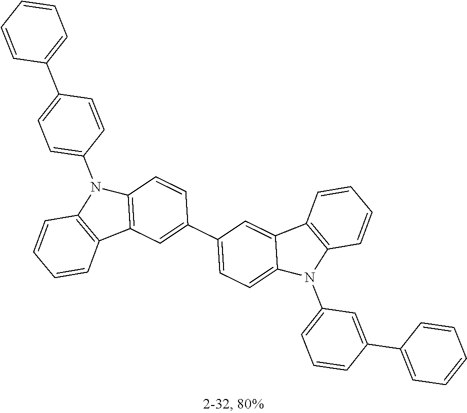 [SMILES: c1ccc(-c2ccc(-n3c4ccccc4c4cc(-c5ccc6c(c5)c5ccccc5n6-c5cccc(-c6ccccc6)c5)ccc43)cc2)cc1] |
| 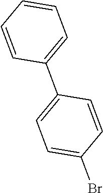 [SMILES: Brc1ccc(-c2ccccc2)cc1] | 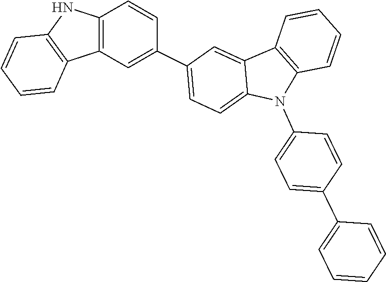 [SMILES: c1ccc(-c2ccc(-n3c4ccccc4c4cc(-c5ccc6[nH]c7ccccc7c6c5)ccc43)cc2)cc1] | 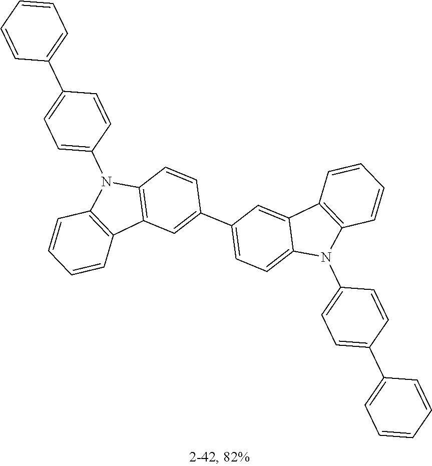 [SMILES: c1ccc(-c2ccc(-n3c4ccccc4c4cc(-c5ccc6c(c5)c5ccccc5n6-c5ccc(-c6ccccc6)cc5)ccc43)cc2)cc1] |

### Preparation Example 3. Preparation of Compound 2-98

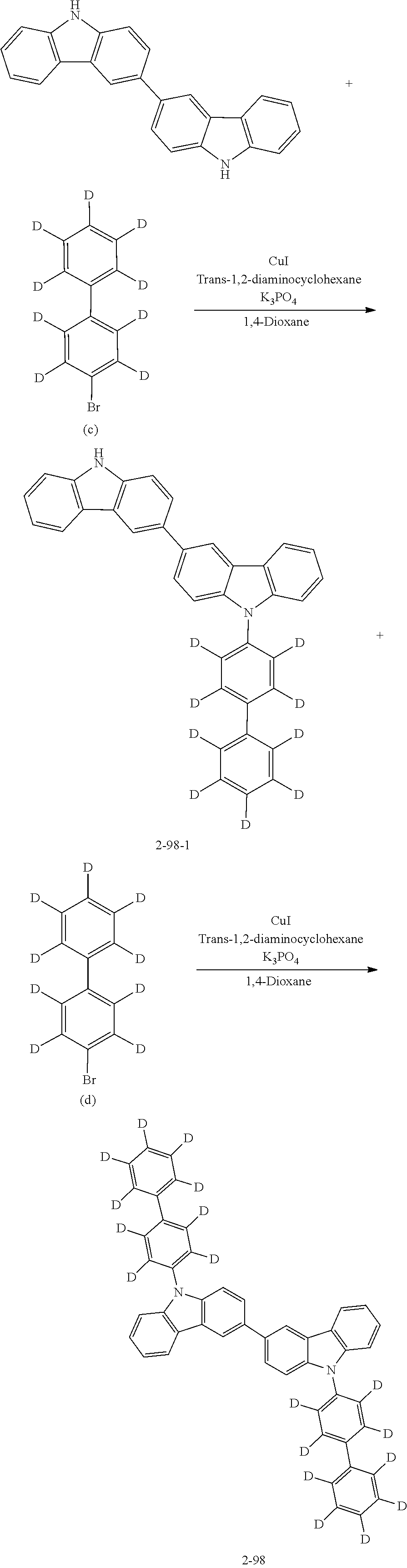 [SMILES: c1ccc2c(c1)[nH]c1ccc(-c3ccc4[nH]c5ccccc5c4c3)cc12 ; [2H]c1c([2H])c([2H])c(-c2c([2H])c([2H])c(Br)c([2H])c2[2H])c([2H])c1[2H] ; [2H]c1c([2H])c([2H])c(-c2c([2H])c([2H])c(-n3c4ccccc4c4cc(-c5ccc6[nH]c7ccccc7c6c5)ccc43)c([2H])c2[2H])c([2H])c1[2H] ; [2H]c1c([2H])c([2H])c(-c2c([2H])c([2H])c(Br)c([2H])c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c([2H])c6[2H])c([2H])c5[2H])ccc43)c([2H])c2[2H])c([2H])c1[2H]]

### Preparation Example 3-1. Preparation of Compound 2-98-1

9H,9′H-3,3′-bicarbazole (10 g, 0.03 mol), 4-bromo-1,1′-biphenyl-2,2′,3,3′,4′,5,5′,6,6′-D₉ (c) (7.26 g, 0.03 mol), copper iodide (CuI) (0.57 g, 0.003 mol), trans-1,2-diaminocyclohexane (0.34 g, 0.003 mol) and K₃PO₄ (12.7 g, 0.06 mol) were dissolved in 1,4-dioxane (100 mL), and then the mixture was refluxed for 8 hours at 125° C.

After the reaction was completed, the result was extracted by introducing dichloromethane thereto at room temperature, and the organic layer was dried with MgSO₄.

The filtered organic layer was concentrated under reduced pressure and then separated by column chromatography to obtain Compound 2-98-1 (13.92 g, yield 94%).

### Preparation Example 3-2. Preparation of Compound 2-98

Compound 2-98-1 (13.92 g, 0.028 mol), 4-bromo-1,1′-biphenyl-2,2′,3,3′,4′,5,5′,6,6′-D₉ (d) (6.83 g, 0.028 mol), copper iodide (CuI) (0.53 g 0.0028 mol), trans-1, 2-diaminocyclohexane (0.32 g, 0.0028 mol) and K₃PO₄ (11.89 g, 0.056 mol) were dissolved in 1,4-dioxane (140 mL), and then the mixture was refluxed for 8 hours at 125° C.

After the reaction was completed, the result was extracted by introducing dichloromethane thereto at room temperature, and the organic layer was dried with MgSO₄.

The filtered organic layer was concentrated under reduced pressure and then separated by column chromatography to obtain Compound 2-98 (16.14 g, yield 88%).

When Compound c and Compound d are the same, 2 equivalents of Compound c may be introduced in the method of Preparation of 2-98-1 in Preparation Example 3 to directly synthesize the target compound.

The following target compounds were synthesized in the same manner as in Preparation Example 3, except that Intermediate c of the following Table 3 was used instead of 4-bromo-1,1′-biphenyl-2,2′,3,3′,4′,5,5′,6,6′-D₉ (c), and Intermediate d of the following Table 3 was used instead of 4-bromo-1,1′-biphenyl-2,2′,3,3′,4′,5,5′,6,6′-D₉ (d).

TABLE 3

| | | Target Compound |
|---|---|---|
| Intermediate c | Intermediate d | (Compound No., Yield) |
| 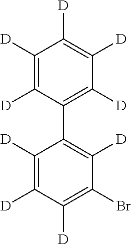 [SMILES: [2H]c1c([2H])c([2H])c(-c2c([2H])c([2H])c([2H])c(Br)c2[2H])c([2H])c1[2H]] | 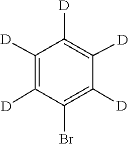 [SMILES: [2H]c1c([2H])c([2H])c(Br)c([2H])c1[2H]] | 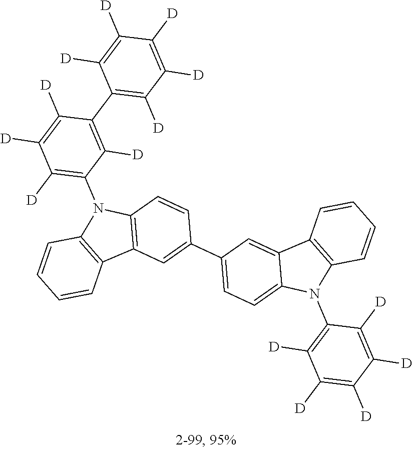 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c([2H])c5[2H])ccc43)c2[2H])c([2H])c1[2H]] |
| 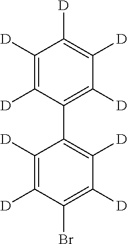 [SMILES: [2H]c1c([2H])c([2H])c(-c2c([2H])c([2H])c(Br)c([2H])c2[2H])c([2H])c1[2H]] | 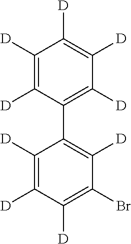 [SMILES: [2H]c1c([2H])c([2H])c(-c2c([2H])c([2H])c([2H])c(Br)c2[2H])c([2H])c1[2H]] | 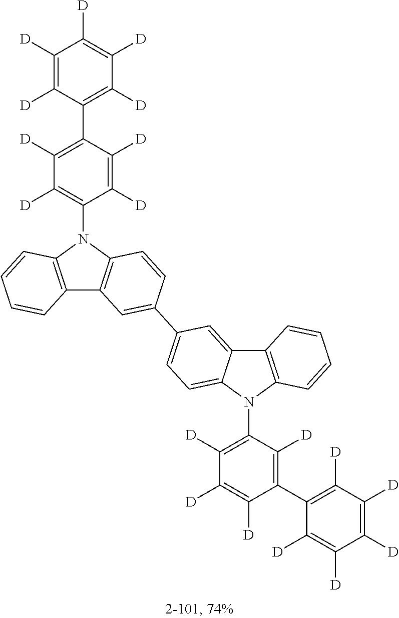 [SMILES: [2H]c1c([2H])c([2H])c(-c2c([2H])c([2H])c(-n3c4ccccc4c4cc(-c5ccc6c(c5)c5ccccc5n6-c5c([2H])c([2H])c([2H])c(-c6c([2H])c([2H])c([2H])c([2H])c6[2H])c5[2H])ccc43)c([2H])c2[2H])c([2H])c1[2H]] |
| 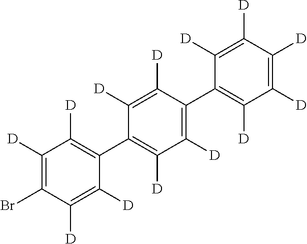 [SMILES: [2H]c1c([2H])c([2H])c(-c2c([2H])c([2H])c(-c3c([2H])c([2H])c(Br)c([2H])c3[2H])c([2H])c2[2H])c([2H])c1[2H]] | 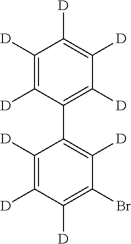 [SMILES: [2H]c1c([2H])c([2H])c(-c2c([2H])c([2H])c([2H])c(Br)c2[2H])c([2H])c1[2H]] | 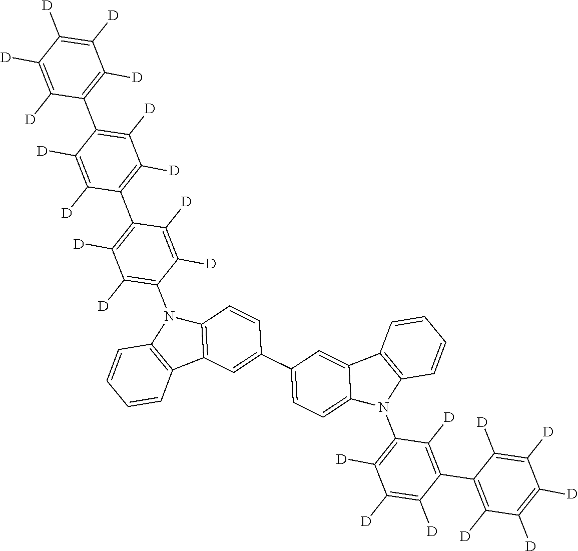 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c([2H])c3[2H])c([2H])c2[2H])c([2H])c1[2H]] |
| | | 2-104, 69% |

### Preparation Example 4. Preparation of Compound 2-106

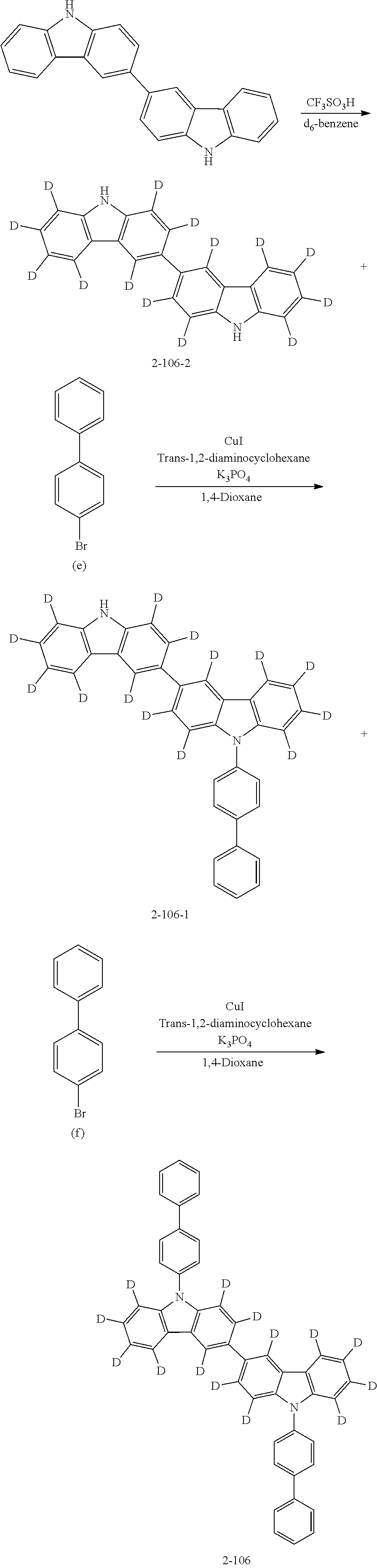

### Preparation Example 4-1. Preparation of Compound 2-106-2

9H,9′H-3,3′-bicarbazole (10 g, 0.03 mol) and triflic acid (34 g, 0.023 mol) were dissolved in D₆-benzene (100 mL), and then the mixture was refluxed for 1 hour at 50° C.

After the reaction was completed, the result was extracted by introducing dichloromethane thereto at room temperature, and the organic layer was dried with MgSO₄.

The filtered organic layer was concentrated under reduced pressure and then separated by column chromatography to obtain Compound 2-106-2 (7.07 g, yield 68%).

### Preparation Example 4-2. Preparation of Compound 2-106-1

Compound 2-106-2 (7.07 g, 0.02 mol), 4-bromo-1, 1′-biphenyl (e) (4.66 g, 0.02 mol), trans-1,2-diaminocyclohexane (0.23 g, 0.002 mol) and K₃PO₄ (8.49 g, 0.04 mol) were dissolved in 1,4-dioxane (70 mL), and then the mixture was refluxed for 8 hours at 125° C.

After the reaction was completed, the result was extracted by introducing distilled water and dichloromethane thereto at room temperature, and after drying the organic layer with MgSO₄, the solvent was removed using a rotary evaporator.

The reaction material was purified by column chromatography (dichloromethane:hexane=1:3 (volume ratio)), and recrystallized with methanol to obtain target Compound 2-106-1 (8.28 g, yield 83%).

### Preparation Example 4-3. Preparation of Compound 2-106

Compound 2-106-1 (8.28 g, 0.017 mol), 4-bromo-1, 1′-biphenyl (f) (3.96 g, 0.017 mol), trans-1, 2-diaminocyclohexane (0.19 g, 0.0017 mol) and K₃PO₄ (7.22 g, 0.034 mol) were dissolved in 1,4-dioxane (80 mL), and then the mixture was refluxed for 8 hours at 125° C.

After the reaction was completed, the result was extracted by introducing distilled water and dichloromethane thereto at room temperature, and after drying the organic layer with MgSO₄, the solvent was removed using a rotary evaporator.

The reaction material was purified by column chromatography (dichloromethane:hexane=1:3 (volume ratio)), and recrystallized with methanol to obtain target Compound 2-106 (8.63 g, yield 78%).

When Compound e and Compound f are the same, 2 equivalents of Compound e may be introduced in the method of Preparation of Compound 2-106-1 in Preparation Example 4 to directly synthesize the target compound.

5 The following target compounds were synthesized in the same manner as in Preparation Example 4, except that Intermediate e of the following Table 4 was used instead of 4-bromo-1,1′-biphenyl (e), and Intermediate f of the following Table 4 was used instead of 4-bromo-1,1′-biphenyl (f).

TABLE 4

| | | Target Compound |
|---|---|---|
| Intermediate e | Intermediate f | (Compound No., Yield) |
| 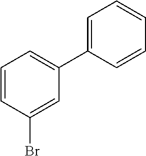 [SMILES: Brc1cccc(-c2ccccc2)c1] | 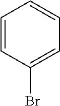 | 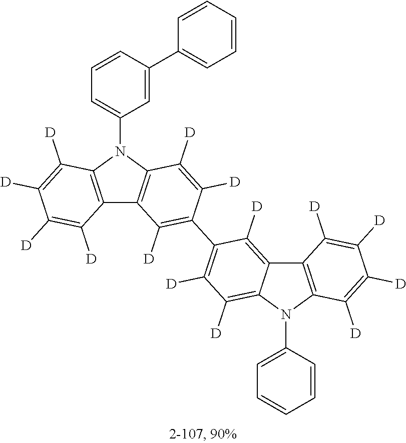 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccccc1] |
| 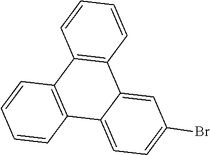 [SMILES: Brc1ccc2c3ccccc3c3ccccc3c2c1] | 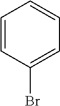 | 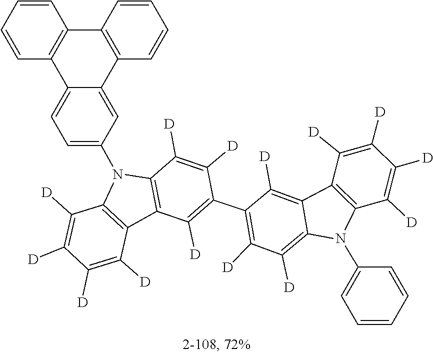 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c5ccccc5c5ccccc5c4c3)c([2H])c([2H])c1n2-c1ccccc1] |

### Preparation Example 5. Preparation of Compound 2-110

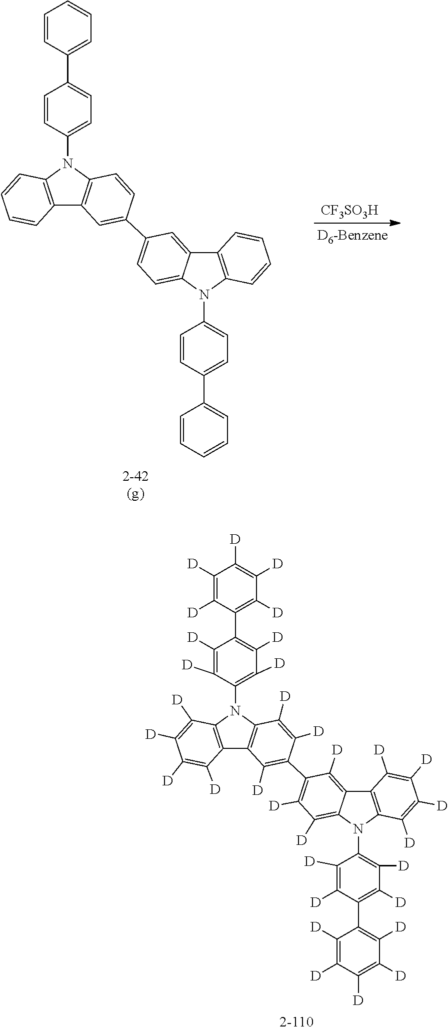 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; "CF3SO3H" ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c(-c6c([2H])c([2H])c([2H])c([2H])c6[2H])c([2H])c5[2H])c([2H])c([2H])c43)c([2H])c2[2H])c([2H])c1[2H]]

Compound 2-42 (12.17 g, 0.017 mol) and triflic acid (40.8 g, 0.27 mol) were dissolved in De-benzene (120 mL), and then the mixture was refluxed for 1 hour at 50° C.

After the reaction was completed, the result was extracted by introducing water and dichloromethane thereto at room temperature, and the organic layer was dried with MgSO₄.

The filtered organic layer was concentrated under reduced pressure and then silica gel filtered. After that, the result was recrystallized with methanol to obtain target Compound 2-110 (8.87 g, yield 78%).

The following target compounds were synthesized in the same manner as in Preparation Example 5, except that Intermediate g of the following Table 5 was used instead of Compound 2-42 (g).

TABLE 5

| Intermediate g | Target Compound |
|---|---|
| (Compound No. ) | (Compound No., Yield) |
| 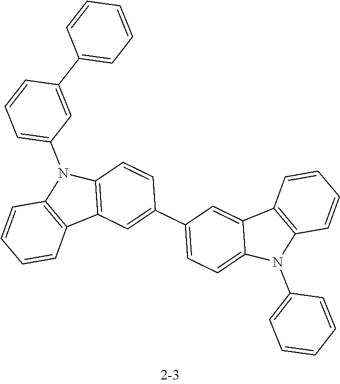 [SMILES: c1ccc(-c2cccc(-n3c4ccccc4c4cc(-c5ccc6c(c5)c5ccccc5n6-c5ccccc5)ccc43)c2)cc1] | 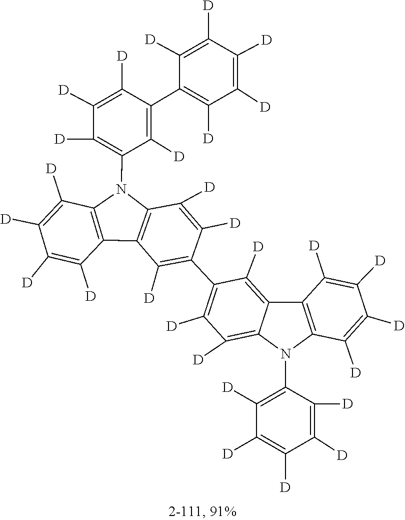 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c2[2H])c([2H])c1[2H]] |
| 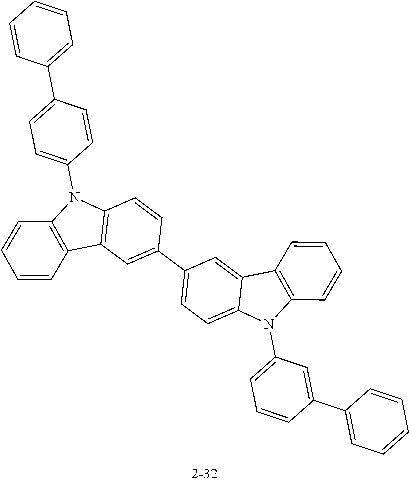 [SMILES: c1ccc(-c2ccc(-n3c4ccccc4c4cc(-c5ccc6c(c5)c5ccccc5n6-c5cccc(-c6ccccc6)c5)ccc43)cc2)cc1] | 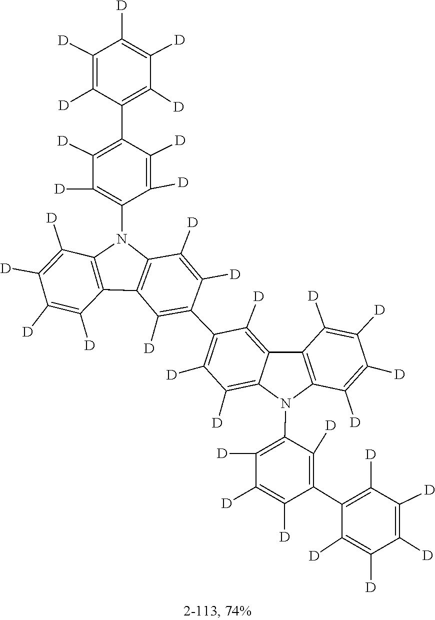 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c(-c6c([2H])c([2H])c([2H])c([2H])c6[2H])c5[2H])c([2H])c([2H])c43)c([2H])c2[2H])c([2H])c1[2H]] |
| 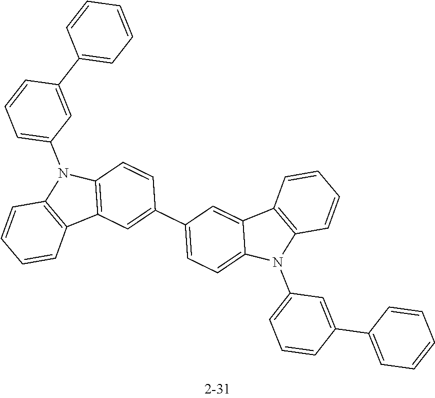 [SMILES: c1ccc(-c2cccc(-n3c4ccccc4c4cc(-c5ccc6c(c5)c5ccccc5n6-c5cccc(-c6ccccc6)c5)ccc43)c2)cc1] | 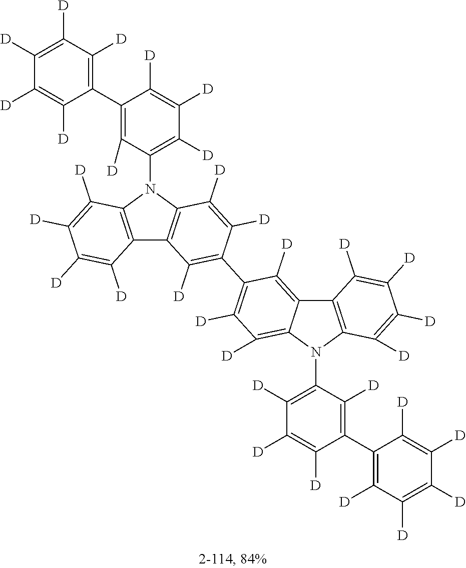 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c(-c6c([2H])c([2H])c([2H])c([2H])c6[2H])c5[2H])c([2H])c([2H])c43)c2[2H])c([2H])c1[2H]] |

Synthesis results for the compounds described in Preparation Examples 1 to 5 and Tables 1 to 5 are shown in the following Table 6 to Table 9.

The following Tables 6 and 7 show measurement values of FD-mass spectrometry (FD-MS: field desorption mass spectrometry), and the following Tables 8 and 9 show measurement values of ¹H NMR (CDCl₃, 200 MHZ).

TABLE 6

| Compound | FD-MS | Compound | FD-MS |
|---|---|---|---|
| 1-1 | m/z = 640.23 (C₄₅H₂₈N₄O = 640.75) | 1-3 | m/z = 716.26 (C₅₁H₃₂N₄O = 716.84) |
| 1-6 | m/z = 730.24 (C₅₁H₃₀N₄O₂ = 730.83) | 1-8 | m/z = 716.26 (C₅₁H₃₂N₄O = 716.84) |
| 1-9 | m/z = 716.26 (C₅₁H₃₂N₄O = 716.84) | 1-13 | m/z = 640.23 (C₄₅H₂₈N₄O = 640.75) |
| 1-17 | m/z = 730.24 (C₅₁H₃₀N₄O₂ = 730.83) | 1-20 | m/z = 716.26 (C₅₁H₃₂N₄O = 716.84) |
| 1-22 | m/z = 716.26 (C₅₁H₃₂N₄O = 716.84) | 1-24 | m/z = 690.24 (C₄₉H₃₀N₄O = 690.81) |
| 1-25 | m/z = 640.23 (C₄₅H₂₈N₄O = 640.75) | 1-31 | m/z = 756.29 (C₅₄H₃₆N₄O = 756.91) |
| 1-33 | m/z = 716.26 (C₅₁H₃₂N₄O = 716.84) | 1-37 | m/z = 640.23 (C₄₅H₂₈N₄O = 640.75) |
| 1-42 | m/z = 730.24 (C₅₁H₃₀N₄O₂ = 730.83) | 1-47 | m/z = 690.24 (C₄₉H₃₀N₄O = 690.81) |
| 1-49 | m/z = 640.23 (C₄₅H₂₈N₄O = 640.75) | 1-51 | m/z = 716.26 (C₅₁H₃₂N₄O = 716.84) |
| 1-53 | m/z = 730.24 (C₅₁H₃₀N₄O₂ = 730.83) | 1-56 | m/z = 716.26 (C₅₁H₃₂N₄O = 716.84) |
| 1-58 | m/z = 716.26 (C₅₁H₃₂N₄O = 716.84) | 1-61 | m/z = 640.23 (C₄₅H₂₈N₄O = 640.75) |
| 1-63 | m/z = 716.26 (C₅₁H₃₂N₄O = 716.84) | 1-68 | m/z = 716.26 (C₅₁H₃₂N₄O = 716.84) |
| 1-71 | m/z = 690.24 (C₄₉H₃₀N₄O = 690.81) | 1-73 | m/z = 640.23 (C₄₅H₂₈N₄O = 640.75) |
| 1-78 | m/z = 730.24 (C₅₁H₃₀N₄O₂ = 730.83) | 1-80 | m/z = 716.26 (C₅₁H₃₂N₄O = 716.84) |
| 1-81 | m/z = 640.23 (C₄₅H₂₈N₄O = 640.75) | 1-82 | m/z = 716.26 (C₅₁H₃₂N₄O = 716.84) |
| 1-87 | m/z = 756.29 (C₅₄H₃₆N₄O = 756.91) | 1-88 | m/z = 716.26 (C₅₁H₃₂N₄O = 716.84) |
| 1-92 | m/z = 730.24 (C₅₁H₃₀N₄O₂ = 730.83) | 1-94 | m/z = 730.24 (C₅₁H₃₀N₄O₂ = 730.83) |
| 1-97 | m/z = 640.23 (C₄₅H₂₈N₄O = 640.75) | 1-99 | m/z = 716.26 (C₅₁H₃₂N₄O = 716.84) |
| 1-102 | m/z = 730.24 (C₅₁H₃₀N₄O₂ = 730.83) | 1-105 | m/z = 640.23 (C₄₅H₂₈N₄O = 640.75) |
| 1-108 | m/z = 730.24 (C₅₁H₃₀N₄O₂ = 730.83) | 1-112 | m/z = 716.26 (C₅₁H₃₂N₄O = 716.84) |
| 1-114 | m/z = 716.26 (C₅₁H₃₂N₄O = 716.84) | 1-115 | m/z = 716.26 (C₅₁H₃₂N₄O = 716.84) |
| 1-117 | m/z = 730.24 (C₅₁H₃₀N₄O₂ = 730.83) | 1-120 | m/z = 716.26 (C₅₁H₃₂N₄O = 716.84) |
| 1-121 | m/z = 640.23 (C₄₅H₂₈N₄O = 640.75) | 1-123 | m/z = 716.26 (C₅₁H₃₂N₄O = 716.84) |
| 1-125 | m/z = 730.24 (C₅₁H₃₀N₄O₂ = 730.83) | 1-127 | m/z = 756.29 (C₅₄H₃₆N₄O = 756.91) |
| 1-131 | m/z = 716.26 (C₅₁H₃₂N₄O = 716.84) | 1-133 | m/z = 730.24 (C₅₁H₃₀N₄O₂ = 730.83) |
| 1-137 | m/z = 640.23 (C₄₅H₂₈N₄O = 640.75) | 1-140 | m/z = 730.24 (C₅₁H₃₀N₄O₂ = 730.83) |
| 1-141 | m/z = 730.24 (C₅₁H₃₀N₄O₂ = 730.83) | 1-143 | m/z = 756.29 (C₅₄H₃₆N₄O = 756.91) |
| 1-145 | m/z = 640.23 (C₄₅H₂₈N₄O = 640.75) | 1-151 | m/z = 756.29 (C₅₄H₃₆N₄O = 756.91) |
| 1-153 | m/z = 640.23 (C₄₅H₂₈N₄O = 640.75) | 1-156 | m/z = 730.24 (C₅₁H₃₀N₄O₂ = 730.83) |
| 1-158 | m/z = 730.24 (C₅₁H₃₀N₄O₂ = 730.83) | 1-163 | m/z = 690.24 (C₄₉H₃₀N₄O = 690.81) |
| 1-165 | m/z = 640.23 (C₄₅H₂₈N₄O = 640.75) | 1-168 | m/z = 730.24 (C₅₁H₃₀N₄O₂ = 730.83) |
| 1-171 | m/z = 756.29 (C₅₄H₃₆N₄O = 756.91) | 1-173 | m/z = 640.23 (C₄₅H₂₈N₄O = 640.75) |
| 1-176 | m/z = 730.24 (C₅₁H₃₀N₄O₂ = 730.83) | 1-179 | m/z = 756.29 (C₅₄H₃₆N₄O = 756.91) |
| 1-182 | m/z = 716.26 (C₅₁H₃₂N₄O = 716.84) | 1-186 | m/z = 730.24 (C₅₁H₃₀N₄O₂ = 730.83) |
| 1-187 | m/z = 756.29 (C₅₄H₃₆N₄O = 756.91) | 1-189 | m/z = 640.23 (C₄₅H₂₈N₄O = 640.75) |
| 1-192 | m/z = 730.24 (C₅₁H₃₀N₄O₂ = 730.83) | 1-194 | m/z = 730.24 (C₅₁H₃₀N₄O₂ = 730.83) |
| 1-196 | m/z = 716.26 (C₅₁H₃₂N₄O = 716.84) | 1-199 | m/z = 716.26 (C₅₁H₃₂N₄O = 716.84) |
| 1-201 | m/z = 730.24 (C₅₁H₃₀N₄O₂ = 730.83) | 1-205 | m/z = 640.23 (C₄₅H₂₈N₄O = 640.75) |
| 1-207 | m/z = 716.26 (C₅₁H₃₂N₄O = 716.84) | 1-212 | m/z = 716.26 (C₅₁H₃₂N₄O = 716.84) |
| 1-213 | m/z = 640.23 (C₄₅H₂₈N₄O = 640.75) | 1-215 | m/z = 716.26 (C₅₁H₃₂N₄O = 716.84) |
| 1-218 | m/z = 730.24 (C₅₁H₃₀N₄O₂ = 730.83) | 1-220 | m/z = 716.26 (C₅₁H₃₂N₄O = 716.84) |
| 1-221 | m/z = 640.23 (C₄₅H₂₈N₄O = 640.75) | 1-222 | m/z = 716.26 (C₅₁H₃₂N₄O = 716.84) |
| 1-223 | m/z = 716.26 (C₅₁H₃₂N₄O = 716.84) | 1-226 | m/z = 730.24 (C₅₁H₃₀N₄O₂ = 730.83) |
| 1-227 | m/z = 756.29 (C₅₄H₃₆N₄O = 756.91) | 1-229 | m/z = 640.23 (C₄₅H₂₈N₄O = 640.75) |
| 1-234 | m/z = 730.24 (C₅₁H₃₀N₄O₂ = 730.83) | 1-236 | m/z = 716.26 (C₅₁H₃₂N₄O = 716.84) |
| 1-237 | m/z = 640.23 (C₄₅H₂₈N₄O = 640.75) | 1-240 | m/z = 730.24 (C₅₁H₃₀N₄O₂ = 730.83) |
| 1-241 | m/z = 730.24 (C₅₁H₃₀N₄O₂ = 730.83) | 1-244 | m/z = 716.26 (C₅₁H₃₂N₄O = 716.84) |
| 1-245 | m/z = 640.23 (C₄₅H₂₈N₄O = 640.75) | 1-247 | m/z = 716.26 (C₅₁H₃₂N₄O = 716.84) |
| 1-251 | m/z = 756.29 (C₅₄H₃₆N₄O = 756.91) | 1-252 | m/z = 716.26 (C₅₁H₃₂N₄O = 716.84) |
| 1-256 | m/z = 730.24 (C₅₁H₃₀N₄O₂ = 730.83) | 1-261 | m/z = 640.23 (C₄₅H₂₈N₄O = 640.75) |
| 1-263 | m/z = 716.26 (C₅₁H₃₂N₄O = 716.84) | 1-265 | m/z = 730.24 (C₅₁H₃₀N₄O₂ = 730.83) |
| 1-268 | m/z = 716.26 (C₅₁H₃₂N₄O = 716.84) | 1-269 | m/z = 640.23 (C₄₅H₂₈N₄O = 640.75) |
| 1-271 | m/z = 716.26 (C₅₁H₃₂N₄O = 716.84) | 1-274 | m/z = 730.24 (C₅₁H₃₀N₄O₂ = 730.83) |
| 1-276 | m/z = 716.26 (C₅₁H₃₂N₄O = 716.84) | 1-277 | m/z = 640.23 (C₄₅H₂₈N₄O = 640.75) |
| 1-282 | m/z = 730.24 (C₅₁H₃₀N₄O₂ = 730.83) | 1-285 | m/z = 640.23 (C₄₅H₂₈N₄O = 640.75) |
| 1-288 | m/z = 730.24 (C₅₁H₃₀N₄O₂ = 730.83) | 1-290 | m/z = 730.24 (C₅₁H₃₀N₄O₂ = 730.83) |
| 1-292 | m/z = 716.26 (C₅₁H₃₂N₄O = 716.84) | 1-293 | m/z = 640.23 (C₄₅H₂₈N₄O = 640.75) |
| 1-296 | m/z = 730.24 (C₅₁H₃₀N₄O₂ = 730.83) | 1-299 | m/z = 756.29 (C₅₄H₃₆N₄O = 756.91) |
| 1-300 | m/z = 716.26 (C₅₁H₃₂N₄O = 716.84) | 1-301 | m/z = 647.27 (C₄₅H₂₁D₇N₄O = 647.79) |
| 1-304 | m/z = 668.40 (C₄₅D₂₈N₄O = 668.92) | 1-306 | m/z = 658.34 (C₄₅H₁₀D₁₈N₄O = 658.86) |
| 1-309 | m/z = 647.27 (C₄₅H₂₁D₇N₄O = 647.79) | 1-312 | m/z = 668.40 (C₄₅D₂₈N₄O = 668.92) |
| 1-315 | m/z = 650.29 (C₄₅H₁₈D₁₀N₄O = 650.81) | 1-317 | m/z = 647.27 (C₄₅H₂₁D₇N₄O = 647.79) |
| 1-322 | m/z = 658.34 (C₄₅H₁₀D₁₈N₄O = 658.86) | 1-328 | m/z = 668.40 (C₄₅D₂₈N₄O = 668.92) |
| 1-332 | m/z = 668.40 (C₄₅D₂₈N₄O = 668.92) | 1-333 | m/z = 647.27 (C₄₅H₂₁DN₄O = 647. 79) |
| 1-338 | m/z = 658.34 (C₄₅H₁₀D₁₈N₄O = 658.86) | 1-344 | m/z = 668.40 (C₄₅D₂₈N₄O = 668.92) |
| 1-349 | m/z = 647.27 (C₄₅H₂₁D₇N₄O = 647.79) | 1-350 | m/z = 658.34 (C₄₅H₁₀D₁₈N₄O = 658.86) |
| 1-356 | m/z = 668.40 (C₄₅D₂₈N₄O = 668.92) | 1-360 | m/z = 668.40 (C₄₅D₂₈N₄O = 668.92) |
| 1-361 | m/z = 647.27 (C₄₅H₂₁D₇N₄O = 647. 79) | 1-366 | m/z = 658.34 (C₄₅H₁₀D₁₈N₄O = 658.86) |
| 1-372 | m/z = 668.40 (C₄₅D₂₈N₄O = 668.92) | 1-374 | m/z = 658.34 (C₄₅H₁₀D₁₈N₄O = 658.86) |
| 1-379 | m/z = 650.29 (C₄₅H₁₈D₁₀N₄O = 650.81) | 1-384 | m/z = 668.40 (C₄₅D₂₈N₄O = 668.92) |
| 1-390 | m/z = 657.33 (C₄₅H₁₁D₁₇N₄O = 657.85) | 1-392 | m/z = 668.40 (C₄₅D₂₈N₄O = 668.92) |
| 1-393 | m/z = 647.27 (C₄₅H₂₁D₇N₄O = 647.79) | 1-398 | m/z = 657.33 (C₄₅H₁₁D₁₇N₄O = 657.85) |
| 1-404 | m/z = 668.40 (C₄₅D₂₈N₄O = 668.92) | 1-405 | m/z = 647.27 (C₄₅H₂₁D₇N₄O = 647. 79) |
| 1-410 | m/z = 658.34 (C₄₅H₁₀D₁₈N₄O = 658.86) | 1-416 | m/z = 668.40 (C₄₅D₂₈N₄O = 668.92) |
| 1-421 | m/z = 647.27 (C₄₅H₂₁D₇N₄O = 647. 79) | 1-426 | m/z = 657.33 (C₄₅H₁₁D₁₇N₄O = 657.85) |
| 1-428 | m/z = 668.40 (C₄₅D₂₈N₄O = 668.92) | 1-430 | m/z = 716.26 (C₅₁H₃₂N₄O = 716.84) |
| 1-436 | m/z = 716.26 (C₅₁H₃₂N₄O = 716.84) | 1-440 | m/z = 716.26 (C₅₁H₃₂N₄O = 716.84) |
| 1-444 | m/z = 730.24 (C₅₁H₃₀N₄O₂ = 730.83) | | |

TABLE 7

| Compound | FD-MS | Compound | FD-MS |
|---|---|---|---|
| 2-3 | m/z = 560.23 (C₄₂H₂₈N₂ = 560.70) | 2-4 | m/z = 560.23 (C₄₂H₂₈N₂ = 560.70) |
| 2-5 | m/z = 636.26 (C₄₈H₃₂N₂ = 636.80) | 2-7 | m/z = 636.26 (C₄₈H₃₂N₂ = 636.80) |
| 2-31 | m/z = 636.26 (C₄₈H₃₂N₂ = 636.80) | 2-32 | m/z = 636.26 (C₄₈H₃₂N₂ = 636.80) |
| 2-42 | m/z = 636.26 (C₄₈H₃₂N₂ = 636.80) | 2-98 | m/z = 654.37 (C₄₈H₁₄D₁₈N₂ = 654.91) |
| 2-99 | m/z = 574.31 (C₄₂H₁₄D₁₄N₂ = 574.79) | 2-101 | m/z = 654.37 (C₄₈H₁₄D₁₈N₂ = 654.91) |
| 2-102 | m/z = 734.43 (C₅₄H₁₄D₂₂N₂ = 735.03) | 2-104 | m/z = 734.43 (C₅₄H₁₄D₂₂N₂ = 735.03) |
| 2-106 | m/z = 650.34 (C₄₈H₁₈D₁₄N₂ = 650.88) | 2-107 | m/z = 574.31 (C₄₂H₁₄D₁₄N₂ = 574.79) |
| 2-108 | m/z = 648.33 (C₄₈H₁₆D₁₄N₂ = 648.87) | 2-110 | m/z = 668.46 (C₄₈D₃₂N₂ = 668.99) |
| 2-111 | m/z = 588.40 (C₄₂D₂₈N₂ = 588.87) | 2-113 | m/z = 668.46 (C₄₈D₃₂N₂ = 668.99) |
| 2-114 | m/z = 668.46 (C₄₈D₃₂N₂ = 668.99) | 2-115 | m/z = 748.51 (C₅₄D₃₂N₂ = 749.12) |
| 2-116 | m/z = 748.51 (C₅₄D₃₂N₂ = 749.12) | | |

TABLE 8

| Compound | ¹H NMR (CDCl₃, 200 MHz) |
|---|---|
| 1-1 | δ = 8.55 (1H, d), 8.36-8.29 (5H, m), 7.99-7.94 (2H, m), 7.82-7.79 (3H, |
| | m), 7.69 (1H, d), 7.57-7.35 (14H, m), 7.25 (1H, d), 7.16 (1H, t) |
| 1-3 | δ = 8.55 (1H, d), 8.38-8.29 (4H, m), 7.99-7.94 (3H, m), 7.82-7.69 (7H, |
| | m), 7.61-7.41 (15H, m), 7.25 (1H, d), 7.16 (1H, d) |
| 1-8 | δ = 8.55 (1H, d), 8.36-8.29 (5H, m), 7.99-7.94 (2H, m), 7.82-7.69 (4H, |
| | m), 7.57-7.41 (14H, m), 7.25 (5H, m), 7.16 (1H, t) |
| 1-13 | δ = 8.55 (1H, d), 8.36-8.29 (5H, m), 7.99-7.75 (7H, m), 7.50-7.41 (13H, |
| | m), 7.25 (1H, d), 7.16 (1H, t) |
| 1-20 | δ = 8.55 (1H, d), 8.36-8.29 (5H, m), 7.99-7.75 (7H, m), 7.55-7.41 (13H, |
| | m), 7.25 (5H, m), 7.16 (1H, t) |
| 1-25 | δ = 8.55 (1H, d), 8.36-8.29 (5H, m), 8.03-7.94 (3H, m), 7.82-7.75 (4H, |
| | m), 7.55-7.35 (13H, m), 7.25 (1H, d), 7.16 (1H, t) |
| 1-31 | δ = 8.55 (1H, d), 8.36-8.29 (3H, m), 8.09-7.89 (6H, m), 7.82-7.75 (5H, |
| | m), 7.55-7.25 (14H, m), 7.16 (1H, t), 1.69 (6H, s) |
| 1-37 | δ = 8.55 (1H, d), 8.36-8.29 (5H, m), 8.02-7.99 (4H, m), 7.55-7.35 (16H, |
| | m), 7.25 (1H, d), 7.16 (1H, t) |
| 1-49 | δ = 8.55 (1H, d), 8.36-8.29 (5H, m), 7.99-7.94 (2H, m), 7.82-7.69 (5H, |
| | m), 7.61-7.31 (14H, m), 7.16 (1H, t) |
| 1-51 | δ = 8.55 (1H, d), 8.38-8.29 (4H, m), 7.99-7.94 (3H, m), 7.79-7.69 (8H, |
| | m), 7.61-7.35 (15H, m), 7.16 (1H, t) |
| 1-53 | δ = 8.55 (1H, d), 8.36-8.29 (3H, m), 8.08 (1H, d), 7.99-7.69 (9H, m), |
| | 7.61-7.31 (15H, m), 7.16 (1H, t) |
| 1-56 | δ = 8.55 (1H, d), 8.36-8.29 (5H, m), 7.99-7.94 (2H, m), 7.82-7.69 (5H, |
| | m), 7.57-7.25 (18H, m), 7.16 (1H, t) |
| 1-61 | δ = 8.55 (1H, d), 8.36-8.29 (5H, m), 7.99-7.74 (8H, m), 7.61 (1H, d), |
| | 7.50-7.31 (12H, m), 7.16 (1H, t) |
| 1-68 | δ = 8.55 (1H, d), 8.36-8.29 (5H, m), 7.99-7.94 (8H, m), 7.61 (1H, d), |
| | 7.50-7.25 (16H, m), 7.16 (1H, t) |
| 1-73 | δ = 8.55 (1H, d), 8.36-8.29 (5H, m), 8.03-7.94 (3H, m), 7.82-7.74 (5H, |
| | m), 7.61 (1H, s), 7.50-7.31 (12H, m), 7.16 (1H, t) |
| 1-78 | δ = 8.55 (1H, d), 8.36-8.29 (3H, m), 8.03-7.94 (4H, m), 7.82-7.69 (7H, |
| | m), 7.61-7.31 (14H, m), 7.16 (1H, t) |
| 1-80 | δ = 8.55 (1H, d), 8.36-8.29 (5H, m), 8.03-7.94 (3H, m), 7.82-7.74 (5H, |
| | m), 7.61 (1H, s), 7.50-7.31 (16H, m), 7.16 (1H, t) |
| 1-82 | δ = 8.55 (1H, d), 8.36-8.29 (3H, m), 8.08-7.94 (6H, m), 7.75-7.74 (3H, |
| | m), 7.61 (1H, s), 7.51-7.25 (17H, m), 7.16 (1H, t) |
| 1-87 | δ = 8.55 (1H, d), 8.36-8.29 (3H, m), 8.09-7.89 (7H, m), 7.78-7.74 (2H, |
| | m), 7.61-7.28 (16H, m), 7.16 (1H, t), 1.69 (6H, s) |
| 1-88 | δ = 8.55 (1H, d), 8.36-8.29 (5H, m), 8.08-7.94 (4H, m), 7.75-7.74 (3H, |
| | m), 7.61 (1H, s), 7.51-7.25 (17H, m), 7.16 (1H, t) |
| 1-92 | δ = 8.55 (1H, d), 8.36-8.29 (3H, m), 8.03-7.94 (5H, m), 7.82-7.69 (6H, |
| | m), 7.54-7.25 (14H, m), 7.16 (1H, t) |
| 1-97 | δ = 8.55 (1H, d), 8.36-8.29 (5H, m), 8.03-7.75 (8H, m), 7.54-7.35 (12H, |
| | m), 7.25 (1H, d), 7.16 (1H, t) |
| 1-99 | δ = 8.55 (1H, d), 8.38-8.29 (4H, m), 7.99-7.75 (12H, m), 7.61 (1H, d), |
| | 7.50-7.35 (12H, m), 7.25 (1H, d), 7.16 (1H, t) |
| 1-105 | δ = 8.55 (1H, d), 8.36-8.29 (5H, m), 8.03-7.94 (4H, m), 7.82-7.75 (4H, |
| | m), 7.54-7.35 (12H, m), 7.25 (1H, d), 7.16 (1H, t) |
| 1-108 | δ = 8.55 (1H, d), 8.36-8.29 (3H, m), 8.03-7.94 (6H, m), 7.82-7.75 (6H, |
| | m), 7.54-7.35 (13H, m), 7.16 (1H, t) |
| 1-112 | δ = 8.55 (1H, d), 8.36-8.29 (5H, m), 8.03-7.94 (4H, m), 7.76-7.75 (4H, |
| | m), 7.54-7.35 (12H, m), 7.25 (5H, m), 7.16 (1H, t) |
| 1-114 | δ = 8.55 (1H, d), 8.36-8.29 (3H, m), 8.08-7.94 (7H, m), 7.75 (2H, d), |
| | 7.50-7.35 (15H, m), 7.25 (3H, m), 7.16 (1H, t) |
| 1-120 | δ = 8.55 (1H, d), 8.36-8.29 (5H, m), 8.08-7.94 (5H, m), 7.75 (2H, d), |
| | 7.54-7.35 (13H, m), 7.25 (5H, m), 7.16 (1H, t) |
| 1-125 | δ = 8.55 (1H, d), 8.36-8.29 (3H, m), 8.08 (1H, d), 7.99-7.79 (8H, m), |
| | 7.69 (1H, d), 7.57-7.31 (15H, m), 7.16 (1H, t) |
| 1-127 | δ = 8.55 (1H, d), 8.36-8.29 (3H, m), 8.09 (1H, d), 7.99-7.89 (5H, m), |
| | 7.82-7.78 (4H, m), 7.69 (1H, d), 7.57-7.28 (14H, m), 7.16 (1H, t), 1.69 |
| | (6H, s) |
| 1-131 | δ = 8.55 (1H, d), 8.38-8.29 (4H, m), 7.99-7.73 (12H, m), 7.61 (1H, d), |
| | 7.50-7.35 (13H, m), 7.16 (1H, t) |
| 1-137 | δ = 8.55 (1H, d), 8.36-8.29 (5H, m), 8.03-7.94 (4H, m), 7.82-7.75 (4H, |
| | m), 7.50-7.35 (13H, m), 7.16 (1H, t) |
| 1-140 | δ = 8.55 (1H, d), 8.36-8.29 (3H, m), 8.03-7.94 (6H, m), 7.82-7.75 (6H, |
| | m), 7.54-7.31 (13H, m), 7.16 (1H, t) |
| 1-143 | δ = 8.55 (1H, d), 8.36-8.29 (3H, m), 8.09-7.90 (7H, m), 7.82-7.75 (5H, |
| | m), 7.50-7.28 (13H, m), 7.16 (1H, t), 1.69 (6H, s) |
| 1-145 | δ = 8.55 (1H, d), 8.36-8.29 (5H, m), 8.08-7.94 (5H, m), 7.51-7.31 (16H, |
| | m), 7.16 (1H, t) |
| 1-153 | δ = 8.55 (1H, d), 8.36-8.31 (5H, m), 7.94-7.91 (2H, m), 7.82-7.69 (5H, |
| | m), 7.57-7.35 (13H, m), 7.25 (1H, d), 7.16 (1H, t) |
| 1-156 | δ = 8.55 (1H, d), 8.36-8.31 (3H, m), 8.03-7.91 (4H, m), 7.82-7.57 (7H, |
| | m), 7.57-7.25 (14H, m), 7.16 (1H, t) |
| 1-158 | δ = 8.55 (1H, d), 8.36-8.31 (3H, m), 7.98-7.91 (3H, m), 7.82-7.69 (7H, |
| | m), 7.57-7.31 (15H, m), 7.16 (1H, t) |
| 1-163 | δ = 8.95 (1H, d), 8.55-8.50 (2H, m), 8.36-8.31 (5H, m), 8.26 (1H, d), |
| | 8.09 (1H, d), 7.94-7.91 (2H, m), 7.82-7.69 (4H, m), 7.57-7.50 (10H, m), |
| | 7.39-7.35 (2H, m), 7.25 (1H, d), 7.16 (1H, t) |
| 1-165 | δ = 8.55 (1H, d), 8.36-8.31 (5H, m), 8.03 (1H, d), 7.94-7.91 (2H, m), |
| | 7.82-7.74 (5H, m), 7.55-7.35 (12H, m), 7.25 (1H, d), 7.16 (1H, t) |
| 1-168 | δ = 8.55 (1H, d), 8.36-8.31 (3H, m), 8.03-7.91 (5H, m), 7.82-7.74 (7H, |
| | m), 7.55-7.25 (13H, m), 7.16 (1H, t) |
| 1-173 | δ = 8.55 (1H, d), 8.36 (4H, m), 7.99-7.89 (3H, m), 7.82-7.77 (4H, m), |
| | 7.69 (1H, d), 7.57-7.35 (13H, m), 7.25 (1H, d), 7.16 (1H, t) |
| 1-176 | δ = 8.55 (1H, d), 8.36 (2H, m), 8.03-7.69 (12H, m), 7.57-7.35 (14H, m), |
| | 7.16 (1H, t) |
| 1-179 | δ = 8.55 (1H, d), 8.36 (2H, m), 8.09 (1H, d), 7.99-7.77 (10H, m), 7.69 |
| | (1H, d), 7.57-7.25 (14H, m), 7.16 (1H, t), 1.69 (6H, s) |
| 1-182 | δ = 8.55 (1H, d), 8.36 (2H, m), 7.99-7.75 (13H, m), 7.55-7.41 (12H, m), |
| | 7.25 (3H, m), 7.16 (1H, t) |
| 1-189 | δ = 8.55 (1H, d), 8.36 (4H, m), 8.03-7.89 (4H, m), 7.82-7.75 (5H, m), |
| | 7.55-7.35 (12H, m), 7.25 (1H, d), 7.16 (1H, t) |
| 1-192 | δ = 8.55 (1H, d), 8.36 (2H, m), 8.03-7.75 (13H, m), 7.55-7.31 (13H, m), |
| | 7.16 (1H, t) |
| 1-196 | δ = 8.55 (1H, d), 8.36 (4H, m), 8.03-7.89 (4H, m), 7.82-7.75 (5H, m), |
| | 7.55-7.35 (12H, m), 7.25 (5H, m), 7.16 (1H, t) |
| 1-199 | δ = 8.55 (1H, d), 8.38-8.36 (3H, m), 8.03-7.89 (6H, m), 7.77-7.73 (4H, |
| | m), 7.55-7.35 (16H, m), 7.25 (1H, d), 7.16 (1H, t) |
| 1-205 | δ = 8.55 (1H, d), 8.36 (4H, m), 7.99-7.94 (3H, m), 7.82-7.69 (6H, m), |
| | 7.57-7.31 (13H, m), 7.16 (1H, t) |
| 1-207 | δ = 8.55 (1H, d), 8.38-8.36 (3H, m), 7.99-7.94 (4H, m), 7.57-7.31 (23H, |
| | m), 7.16 (1H, t) |
| 1-212 | δ = 8.55 (1H, d), 8.36 (4H, m), 7.99-7.94 (3H, m), 7.82-7.69 (6H, m), |
| | 7.57-7.25 (17H, m), 7.16 (1H, t) |
| 1-213 | δ = 8.55 (1H, d), 8.36 (4H, m), 7.99-7.74 (10H, m), 7.61 (1H, s), |
| | 7.50-7.31 (11H, m), 7.16 (1H, t) |
| 1-220 | δ = 8.55 (1H, d), 8.36 (4H, m), 7.99-7.73 (12H, m), 7.61 (3H, m), |
| | 7.50-7.31 (11H, m), 7.16 (1H, t) |
| 1-221 | δ = 8.55 (1H, d), 8.36 (4H, m), 8.03-7.89 (4H, m), 7.82-7.74 (6H, m), |
| | 7.61 (1H, s), 7.50-7.31 (11H, m), 7.16 (1H, t) |
| 1-222 | δ = 8.55 (1H, d), 8.36 (2H, m), 8.03-7.89 (6H, m), 7.82-7.74 (8H, m), |
| | 7.61 (1H, s), 7.50-7.25 (13H, m), 7.16 (1H, t) |
| 1-229 | δ = 8.55 (1H, d), 8.36 (4H, m), 8.08-7.89 (5H, m), 7.77-7.74 (2H, m), |
| | 7.61 (1H, s), 7.50-7.31 (14H, m), 7.16 (1H, t) |
| 1-236 | δ = 8.55 (1H, d), 8.36 (4H, m), 8.08-7.89 (5H, m), 7.77-7.74 (4H, m), |
| | 7.61 (1H, s), 7.50-7.25 (16H, m), 7.16 (1H, t) |
| 1-237 | δ = 8.55 (1H, d), 8.36 (4H, m), 7.99-7.89 (4H, m), 7.82-7.77 (4H, m), |
| | 7.69 (1H, d), 7.50-7.35 (12H, m), 7.25 (1H, d), 7.16 (1H, t) |
| 1-240 | δ = 8.55 (1H, d), 8.36 (2H, m), 8.03-7.69 (13H, m), 7.57-7.25 (13H, m), |
| | 7.16 (1H, t) |
| 1-244 | δ = 8.55 (1H, d), 8.36 (4H, m), 7.99-7.89 (4H, m), 7.82-7.75 (5H, m), |
| | 7.57-7.35 (12H, m), 7.25 (5H, m), 7.16 (1H, t) |
| 1-245 | δ = 8.55 (1H, d), 8.36 (4H, m), 7.99-7.75 (10H, m), 7.54-7.35 (11H, m), |
| | 7.25 (1H, d), 7.16 (1H, t) |
| 1-247 | δ = 8.55 (1H, d), 8.38-8.36 (3H, m), 7.99-7.73 (14H, m), 7.61 (1H, d), |
| | 7.54-7.35 (11H, m), 7.25 (1H, d), 7.16 (1H, t) |
| 1-252 | δ = 8.55 (1H, d), 8.36 (4H, m), 7.99-7.75 (12H, m), 7.61 (2H, m), |
| | 7.54-7.35 (11H, m), 7.25 (1H, d), 7.16 (1H, t) |
| 1-256 | δ = 8.55 (1H, d), 8.36 (2H, m), 8.03-7.75 (14H, m), 7.54-7.25 (12H, m), |
| | 7.16 (1H, t) |
| 1-261 | δ = 8.55 (1H, d), 8.36 (4H, m), 8.08-7.94 (6H, m), 7.77 (1H, d), |
| | 7.54-7.35 (14H, m), 7.25 (1H, d), 7.16 (1H, t) |
| 1-263 | δ = 8.55 (1H, d), 8.38-8.36 (3H, m), 8.08-7.89 (7H, m), 7.77-7.74 (4H, |
| | m), 7.61 (1H, d), 7.54-7.35 (14H, m), 7.25 (1H, d), 7.16 (1H, t) |
| 1-268 | δ = 8.55 (1H, d), 8.36 (4H, m), 8.08-7.89 (6H, m), 7.77-7.75 (3H, m), |
| | 7.54-7.31 (12H, m), 7.25 (5H, m), 7.16 (1H, t) |
| 1-269 | δ = 8.55 (1H, d), 8.36 (4H, m), 7.99-7.89 (4H, m), 7.82-7.77 (4H, m), |
| | 7.69 (1H, d), 7.57-7.31 (13H, m), 7.16 (1H, t) |
| 1-271 | δ = 8.55 (1H, d), 8.38-8.36 (3H, m), 7.99-7.89 (5H, m), 7.82-7.73 (8H, |
| | m), 7.61-7.31 (14H, m), 7.16 (1H, t) |
| 1-276 | δ = 8.55 (1H, d), 8.36 (4H, m), 7.99-7.89 (4H, m), 7.82-7.69 (5H, m), |
| | 7.57-7.25 (17H, m), 7.16 (1H, t) |
| 1-277 | δ = 8.55 (1H, d), 8.36 (4H, m), 7.99-7.75 (10H, m), 7.50-7.31 (12H, m), |
| | 7.16 (1H, t) |
| 1-285 | δ = 8.55 (1H, d), 8.36 (4H, m), 8.03-7.89 (5H, m), 7.82-7.75 (5H, m), |
| | 7.50-7.31 (12H, m), 7.16 (1H, t) |
| 1-288 | δ = 8.55 (1H, d), 8.36 (2H, m), 8.03-7.75 (14H, m), 7.54-7.31 (12H, m), |
| | 7.16 (1H, t) |
| 1-292 | δ = 8.55 (1H, d), 8.36 (4H, m), 8.03-7.89 (6H, m), 7.82-7.75 (6H, m), |
| | 7.69 (2H, m), 7.50-7.31 (12H, m), 7.16 (1H, t) |
| 1-293 | δ = 8.55 (1H, d), 8.36 (4H, m), 8.08-7.89 (6H, m), 7.77 (1H, d), |
| | 7.51-7.31 (15H, m), 7.16 (1H, t) |
| 1-296 | δ = 8.55 (1H, d), 8.36 (2H, m), 8.08-7.89 (8H, m), 7.82-7.77 (3H, m), |
| | 7.54-7.31 (15H, m), 7.16 (1H, t) |
| 1-300 | δ = 8.55 (1H, d), 8.36 (4H, m), 8.08-7.89 (6H, m), 7.77-7.75 (3H, m), |
| | 7.51-7.25 (17H, m), 7.16 (1H, t) |
| 1-306 | δ = 8.36 (4H, m), 7.50 (6H, m) |
| 1-309 | δ = 8.36 (4H, m), 8.03 (1H, d), 7.82-7.75 (4H, m), 7.55-7.41 (11H, m), |
| | 7.25 (1H, d) |
| 1-312 | δ = No ¹H NMR peaks due to 100% deuterium content |
| 1-317 | δ = 8.36 (4H, m), 7.82-7.69 (5H, m), 7.61-7.41 (11H, m), 7.31 (1H, d) |
| 1-322 | δ = 8.36 (4H, m), 7.50 (6H, m) |
| 1-328 | δ = No ¹H NMR peaks due to 100% deuterium content |
| 1-333 | δ = 8.36 (4H, m), 7.98 (1H, d), 7.82-7.79 (3H, m), 7.69 (1H, d), 7.57-7.41 |
| | (11H, m), 7.25 (1H, d) |
| 1-338 | δ = 8.36 (4H, m), 7.50 (6H, m) |
| 1-344 | δ = No ¹H NMR peaks due to 100% deuterium content |
| 1-349 | δ = 8.36 (4H, m), 7.94-7.75 (6H, m), 7.50-7.41 (10H, m), 7.31 (1H, d) |
| 1-350 | δ = 8.36 (4H, m), 7.50 (6H, m) |
| 1-356 | δ = No ¹H NMR peaks due to 100% deuterium content |
| 1-361 | δ = 8.36 (4H, m), 7.82-7.79 (3H, m), 7.69 (1H, d), 7.57-7.44 (12H, m), |
| | 7.25 (1H, d) |
| 1-366 | δ = 8.36 (4H, m), 7.50 (6H, m) |
| 1-372 | δ = No ¹H NMR peaks due to 100% deuterium content |
| 1-374 | δ = 8.36 (4H, m), 7.50 (6H, m) |
| 1-379 | δ = 8.55 (1H, d), 8.31 (1H, d), 7.98-7.91 (3H, m), 7.82-7.69 (5H, m), |
| | 7.57-7.35 (6H, m), 7.25 (1H, d), 7.16 (1H, t) |
| 1-384 | δ = No ¹H NMR peaks due to 100% deuterium content |
| 1-390 | δ = 8.36 (4H, m), 7.50 (6H, m) |
| 1-392 | δ = No ¹H NMR peaks due to 100% deuterium content |
| 1-393 | δ = 8.36 (4H, m), 7.88-7.74 (6H, m), 7.61 (1H, s), 7.50-7.41 (9H, m), |
| | 7.31 (1H, d) |
| 1-398 | δ = 8.36 (4H, m), 7.50 (6H, m) |
| 1-404 | δ = No ¹H NMR peaks due to 100% deuterium content |
| 1-405 | δ = 8.36 (4H, m), 7.98 (1H, d), 7.82-7.79 (3H, m), 7.69 (1H, d), |
| | 7.57-7.41 (11H, m), 7.25 (1H, d) |
| 1-410 | δ = 8.36 (4H, m), 7.50 (6H, m) |
| 1-416 | δ = No ¹H NMR peaks due to 100% deuterium content |
| 1-421 | δ = 8.36 (4H, m), 7.94 (1H, d), 7.82-7.79 (3H, m), 7.69 (1H, d), |
| | 7.57-7.41 (11H, m), 7.25 (1H, d) |
| 1-426 | δ = 8.36 (4H, m), 7.50 (6H, m) |
| 1-428 | δ = No ¹H NMR peaks due to 100% deuterium content |
| 1-430 | δ = 8.55 (1H, d), 8.36-8.31 (3H, m), 7.98-7.91 (5H, m), 7.82-7.69 (7H, |
| | m), 7.57-7.35 (12H, m), 7.25-7.16 (4H, m) |
| 1-436 | δ = 8.55 (1H, d), 8.36-8.31 (5H, m), 7.98-7.91 (3H, m), 7.82-7.69 (5H, |
| | m), 7.57-7.35 (12H, m), 7.25-7.16 (6H, m) |
| 1-440 | δ = 8.55 (1H, d), 8.36-8.31 (5H, m), 8.03-7.91 (4H, m), 7.82-7.74 (5H, |
| | m), 7.54-7.35 (11H, m), 7.25 (5H, m), 7.16 (1H, t) |
| 1-444 | δ = 8.55 (1H, d), 8.36-8.31 (3H, m), 8.03-7.91 (6H, m), 7.82-7.74 (7H, |
| | m), 7.54-7.25 (12H, m), 7.16 (1H, t) |

TABLE 9

| Compound | ¹H NMR (CDCl₃, 200 MHz) |
|---|---|
| 2-3 | δ = 8.55 (1H, d), 8.30 (1H, d), 8.21-8.13 (3H, m), 7.99-7.89 (4H, m), |
| | 7.77-7.35 (17H, m), 7.20-7.16 (2H, m) |
| 2-4 | δ = 8.55 (1H, d), 8.30 (1H, d), 8.19-8.13 (2H, m), 7.99-7.89 (8H, m), |
| | 7.77-7.75 (3H, m), 7.62-7.35 (11H, m), 7.20-7.16 (2H, m) |
| 2-5 | δ = 8.55 (1H, d), 8.30 (1H, d), 8.21-8.13 (3H, m), 7.99-7.94 (2H, m), |
| | 7.89 (2H, s), 7.77-7.35 (17H, m), 7.25-7.16 (6H, m) |
| 2-7 | δ = 8.55 (1H, d), 8.31-8.30 (3H, d), 8.19-8.13 (2H, m), 7.99-7.89 (5H, |
| | m), 7.77-7.75 (5H, m), 7.62-7.35 (14H, m), 7.20-7.16 (2H, m) |
| 2-31 | δ = 8.55 (1H, d), 8.30 (1H, d), 8.21-8.13 (4H, m), 7.99-7.89 (4H, m), |
| | 7.77-7.35 (20H, m), 7.20-7.16 (2H, m) |
| 2-32 | δ = 8.55 (1H, d), 8.30 (1H, d), 8.21-8.13 (3H, m), 7.99-7.89 (8H, m), |
| | 7.77-7.35 (17H, m), 7.20-7.16 (2H, m) |
| 2-37 | δ = 8.55 (1H, d), 8.30 (1H, d), 8.21-8.13 (3H, m), 8.03-7.35 (23H, m), |
| | 7.20-7.16 (2H, m) |
| 2-42 | δ = 8.55 (1H, d), 8.30 (1H, d), 8.19 (1H, d) 8.13 (1H, d), 7.99-7.89 (12H, |
| | m), 7.77-7.75 (5H, m), 7.50-7.35 (8H, m), 7.20-7.16 (2H, m) |
| 2-82 | δ = 8.55 (1H, d), 8.30 (1H, d), 8.19-8.13 (2H, m), 8.03-7.77 (13H, m), |
| | 7.59-7.50 (6H, m) 7.36-7.35 (3H, m), 7.20-7.16 (2H, m) |
| 2-98 | δ = 8.55 (1H, d), 8.30 (1H, d), 8.13~8.19 (2H, m), 7.89~7.99 (4H, m), |
| | 7.77 (1H, d), 7.50~7.58 (2H, m), 7.35 (1H, t), 7.16~7.20 (2H, t) |
| 2-99 | δ = 8.55 (1H, d), 8.30 (1H, d), 8.13~8.19 (2H, m), 7.89~7.99 (4H, m), |
| | 7.77 (1H, d), 7.50~7.58 (2H, m), 7.35 (1H, t), 7.16~7.20 (2H, t) |
| 2-101 | δ = 8.55 (1H, d), 8.30 (1H, d), 8.13~8.19 (2H, m), 7.89~7.99 (4H, m), |
| | 7.77 (1H, d), 7.50~7.58 (2H, m), 7.35 (1H, t), 7.16~7.20 (2H, t) |
| 2-104 | δ = 8.55 (1H, d), 8.30 (1H, d), 8.13~8.19 (2H, m), 7.89~7.99 (4H, m), |
| | 7.77 (1H, d), 7.50~7.58 (2H, m), 7.35 (1H, t), 7.16~7.20 (2H, t) |
| 2-106 | δ = 7.91~7.92 (8H, m), 7.75 (4H, d), 7.41~7.49 (6H, m) |
| 2-107 | δ = 8.21 (1H, s), 7.41~7.68 (13H, m) |
| 2-108 | δ = 9.05 (1H, s), 8.33~8.25 (4H, m), 7.94 (1H, d), 7.70~7.50 (10H, m) |
| 2-110 | δ = No ¹H NMR peaks due to 100% deuterium content |
| 2-111 | δ = No ¹H NMR peaks due to 100% deuterium content |
| 2-113 | δ = No ¹H NMR peaks due to 100% deuterium content |
| 2-114 | δ = No ¹H NMR peaks due to 100% deuterium content |
| 2-115 | δ = No ¹H NMR peaks due to 100% deuterium content |
| 2-116 | δ = No ¹H NMR peaks due to 100% deuterium content |

### Experimental Example 1

### Experimental Example 1-1. Manufacture of Organic Light Emitting Device

A glass substrate coated with indium tin oxide (ITO) as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and then subjected to UVO (ultraviolet ozone) treatment for 5 minutes using UV (ultraviolet) in a UV cleaner. After that, the substrate was transferred to a plasma cleaner (PT), and after subjected to plasma treatment under vacuum for ITO work function increase and residual film removal, the substrate was transferred to a thermal deposition apparatus for organic deposition.

On the transparent ITO electrode (positive electrode), a hole injection layer 2-TNATA (4,4′,4″-tris[2-naphthyl(phenyl)amino]triphenylamine) was formed to 100 Å and a hole transport layer NPB (N,N′-di(1-naphthyl)-N,N′-diphenyl-(1,1′-biphenyl)-4,4′-diamine) was formed to 1100 Å as common layers.

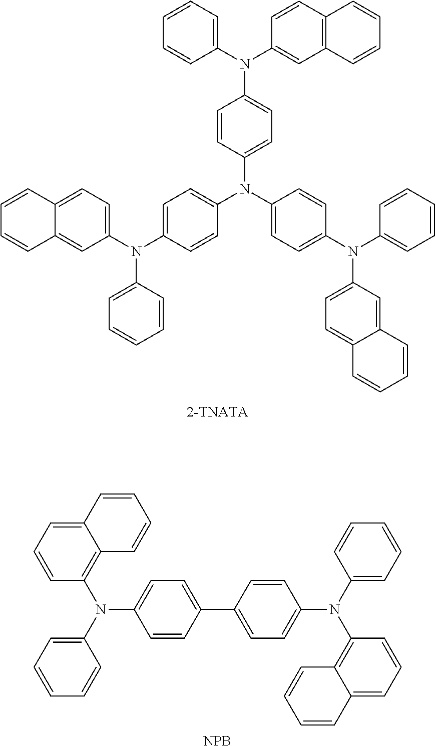 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

A light emitting layer was thermal vacuum deposited thereon as follows. The light emitting layer was deposited by depositing a compound of Chemical Formula 1 described in the following Table 10 to a thickness of 400 Å as a host, and doping the host with a green phosphorescent dopant Ir(ppy)₃ by 7% by weight with respect to the host weight. After that, BCP was deposited to a thickness of 60 Å as a hole blocking layer, and Alq₃ was deposited to a thickness of 200 Å thereon as an electron transport layer. Lastly, an electron injection layer was formed on the electron transport layer by depositing lithium fluoride (LiF) to a thickness of 10 Å, and then a negative electrode was formed on the electron injection layer by depositing aluminum (Al) to a thickness of 1,200 Å, and as a result, an organic light emitting device was manufactured.

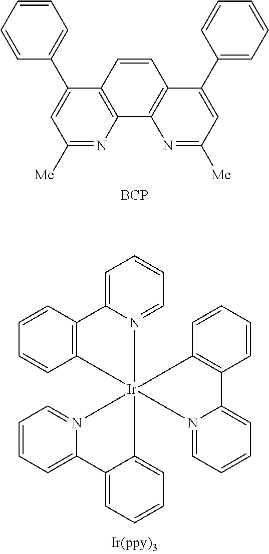

Meanwhile, all the organic compounds required to manufacture the OLED device were vacuum sublimation purified under 10-8 torr to 10-6 torr for each material to be used in the OLED manufacture.

### Experimental Example 1-2. Driving Voltage and Light Emission Efficiency of Organic Light Emitting Device

For each of the organic light emitting devices of Examples 1 to 61 and Comparative Examples 1 to 10 manufactured as above, electroluminescent (EL) properties were measured using M7000 manufactured by McScience Inc., and with the measurement results, T90 was measured when standard luminance was 6,000 cd/m² through a lifetime measurement system (M6000) manufactured by McScience Inc.

Properties of the organic electroluminescent devices of the present disclosure are as shown in the following Table 10. The T90 means a lifetime (unit: h, hour), a time taken for luminance to become 90% with respect to initial luminance.

TABLE 10

| | | Driving | | Color | Life |
|---|---|---|---|---|---|
| | Light Emitting | Voltage | Efficiency | Coordinate | time |
| | Layer Compound | (V) | (cd/A) | (x, y) | (T₉₀) |
| Comparative | C | 5.27 | 55.8 | (0.278, 0.673) | 79 |
| Example 1 | | | | | |
| Comparative | D | 5.24 | 56.9 | (0.282, 0.676) | 76 |
| Example 2 | | | | | |
| Comparative | E | 5.13 | 59.3 | (0.284, 0.674) | 62 |
| Example 3 | | | | | |
| Comparative | F | 5.49 | 58.7 | (0.281, 0.685) | 68 |
| Example 4 | | | | | |
| Comparative | G | 5.42 | 38.8 | (0.276, 0.682) | 61 |
| Example 5 | | | | | |
| Comparative | H | 5.51 | 60.5 | (0.282, 0.679) | 72 |
| Example 6 | | | | | |
| Comparative | I | 5.08 | 57.0 | (0.275, 0.672) | 87 |
| Example 7 | | | | | |
| Comparative | J | 5.03 | 56.5 | (0.279, 0.676) | 79 |
| Example 8 | | | | | |
| Comparative | K | 5.02 | 57.8 | (0.285, 0.680) | 85 |
| Example 9 | | | | | |
| Comparative | L | 5.05 | 55.3 | (0.277, 0.682) | 80 |
| Example 10 | | | | | |
| Example 1 | 1-20 | 4.23 | 66.5 | (0.274, 0.681) | 125 |
| Example 2 | 1-25 | 4.26 | 67.1 | (0.275, 0.686) | 123 |
| Example 3 | 1-37 | 4.32 | 66.8 | (0.273, 0.674) | 128 |
| Example 4 | 1-56 | 4.42 | 67.3 | (0.278, 0.682) | 130 |
| Example 5 | 1-68 | 4.40 | 67.2 | (0.276, 0.686) | 128 |
| Example 6 | 1-82 | 4.43 | 68.3 | (0.275, 0.684) | 126 |
| Example 7 | 1-92 | 4.39 | 70.2 | (0.271, 0.691) | 132 |
| Example 8 | 1-114 | 4.40 | 69.1 | (0.272, 0.672) | 135 |
| Example 9 | 1-120 | 4.38 | 69.8 | (0.276, 0.682) | 138 |
| Example 10 | 1-131 | 4.44 | 67.5 | (0.274, 0.684) | 125 |
| Example 11 | 1-140 | 4.53 | 66.3 | (0.272, 0.681) | 128 |
| Example 12 | 1-143 | 4.42 | 67.7 | (0.278, 0.685) | 130 |
| Example 13 | 1-156 | 4.59 | 64.2 | (0.271, 0.690) | 114 |
| Example 14 | 1-165 | 4.58 | 63.1 | (0.273, 0.685) | 111 |
| Example 15 | 1-168 | 4.52 | 65.2 | (0.277, 0.668) | 116 |
| Example 16 | 1-430 | 4.53 | 65.7 | (0.272, 0.681) | 115 |
| Example 17 | 1-436 | 4.57 | 68.3 | (0.274, 0.678) | 113 |
| Example 18 | 1-444 | 4.58 | 67.4 | (0.279, 0.679) | 119 |
| Example 19 | 1-182 | 4.18 | 68.3 | (0.269, 0.669) | 138 |
| Example 20 | 1-192 | 4.21 | 69.2 | (0.277, 0.681) | 141 |
| Example 21 | 1-199 | 4.19 | 70.5 | (0.275, 0.676) | 135 |
| Example 22 | 1-207 | 4.24 | 69.6 | (0.272, 0.690) | 137 |
| Example 23 | 1-212 | 4.27 | 69.8 | (0.268, 0.667) | 140 |
| Example 24 | 1-222 | 4.23 | 68.3 | (0.271, 0.672) | 137 |
| Example 25 | 1-240 | 4.26 | 70.1 | (0.275, 0.683) | 135 |
| Example 26 | 1-247 | 4.25 | 70.6 | (0.279, 0.684) | 139 |
| Example 27 | 1-256 | 4.19 | 69.5 | (0.281, 0.675) | 141 |
| Example 28 | 1-276 | 4.21 | 68.3 | (0.272, 0.689) | 138 |
| Example 29 | 1-288 | 4.23 | 69.3 | (0.268, 0.685) | 137 |
| Example 30 | 1-296 | 4.30 | 70.4 | (0.272, 0.681) | 135 |
| Example 31 | 1-306 | 3.55 | 78.6 | (0.277, 0.681) | 157 |
| Example 32 | 1-309 | 3.75 | 75.8 | (0.269, 0.672) | 152 |
| Example 33 | 1-312 | 3.42 | 81.3 | (0.276, 0.673) | 161 |
| Example 34 | 1-317 | 3.79 | 75.3 | (0.277, 0.682) | 151 |
| Example 35 | 1-322 | 3.57 | 78.1 | (0.272, 0.690) | 156 |
| Example 36 | 1-328 | 3.48 | 80.6 | (0.275, 0.685) | 160 |
| Example 37 | 1-333 | 3.72 | 75.6 | (0.274, 0.671) | 153 |
| Example 38 | 1-338 | 3.55 | 78.8 | (0.277, 0.682) | 157 |
| Example 39 | 1-344 | 3.41 | 81.6 | (0.265, 0.663) | 162 |
| Example 40 | 1-349 | 3.76 | 73.8 | (0.278, 0.682) | 152 |
| Example 41 | 1-350 | 3.58 | 77.3 | (0.278, 0.683) | 156 |
| Example 42 | 1-356 | 3.45 | 80.2 | (0.274, 0.677) | 160 |
| Example 43 | 1-361 | 3.98 | 74.3 | (0.269, 0.669) | 140 |
| Example 44 | 1-366 | 3.81 | 76.8 | (0.278, 0.683) | 142 |
| Example 45 | 1-372 | 3.66 | 78.1 | (0.273, 0.686) | 151 |
| Example 46 | 1-374 | 3.78 | 77.3 | (0.273, 0.691) | 146 |
| Example 47 | 1-379 | 3.95 | 75.2 | (0.265, 0.663) | 142 |
| Example 48 | 1-384 | 3.61 | 78.4 | (0.276, 0.671) | 153 |
| Example 49 | 1-390 | 3.49 | 79.1 | (0.278, 0.682) | 163 |
| Example 50 | 1-392 | 3.32 | 80.9 | (0.273, 0.688) | 168 |
| Example 51 | 1-393 | 3.69 | 75.2 | (0.276, 0.671) | 159 |
| Example 52 | 1-398 | 3.52 | 78.7 | (0.281, 0.690) | 162 |
| Example 53 | 1-404 | 3.32 | 80.6 | (0.278, 0.683) | 167 |
| Example 54 | 1-405 | 3.62 | 76.8 | (0.273, 0.686) | 162 |
| Example 55 | 1-410 | 3.46 | 79.4 | (0.273, 0.691) | 166 |
| Example 56 | 1-416 | 3.25 | 82.9 | (0.269, 0.669) | 171 |
| Example 57 | 1-421 | 3.67 | 75.9 | (0.278, 0.683) | 160 |
| Example 58 | 1-426 | 3.50 | 79.0 | (0.273, 0.686) | 164 |
| Example 59 | 1-428 | 3.31 | 81.2 | (0.273, 0.691) | 169 |
| Example 60 | A | 4.83 | 59.8 | (0.282, 0.674) | 98 |
| Example 61 | B | 4.90 | 58.3 | (0.283, 0.672) | 96 |

[Compounds A to L]

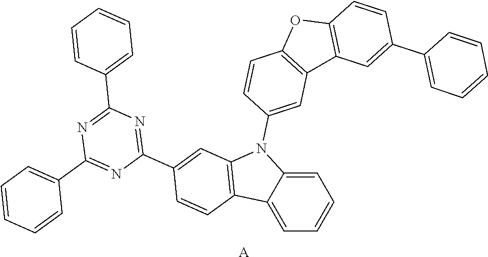

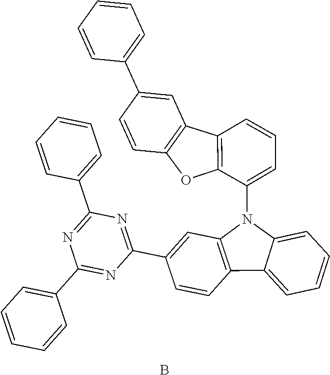

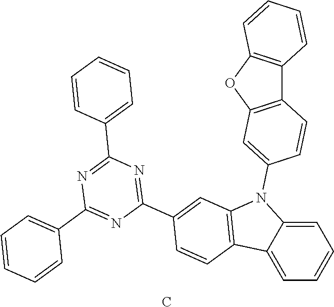

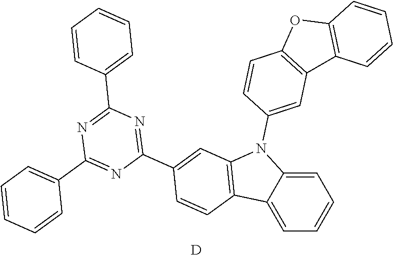

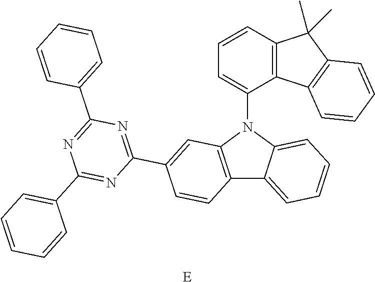

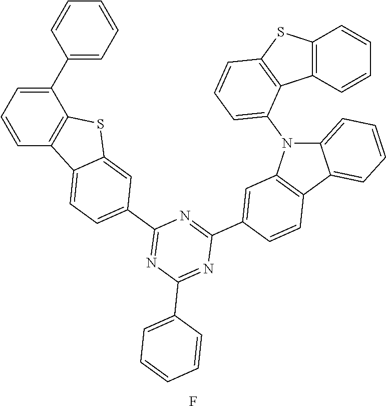

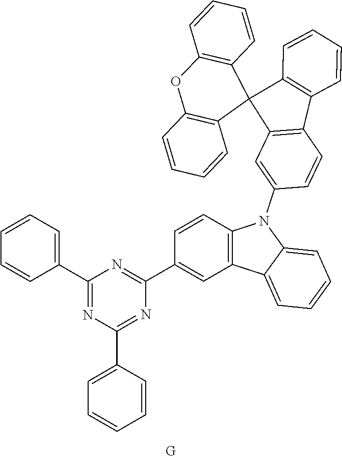 [SMILES: c1ccc(-c2nc(-c3ccccc3)nc(-c3ccc4c(c3)c3ccccc3n4-c3ccc4c(c3)C3(c5ccccc5Oc5ccccc53)c3ccccc3-4)n2)cc1]

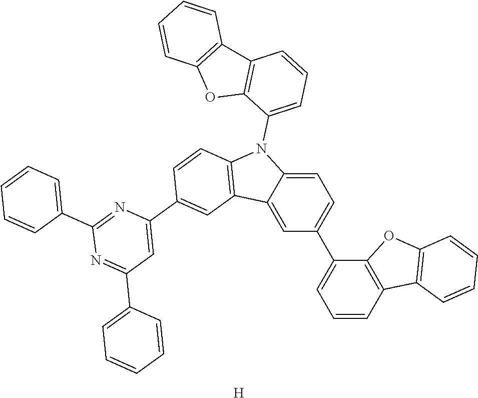

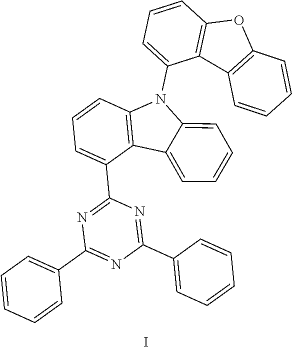

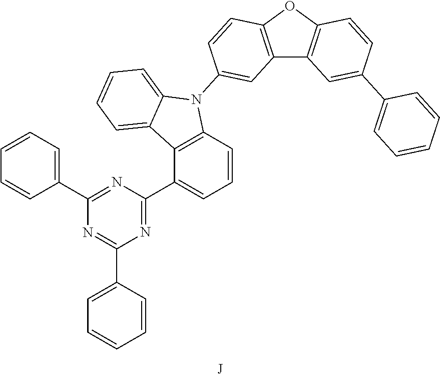

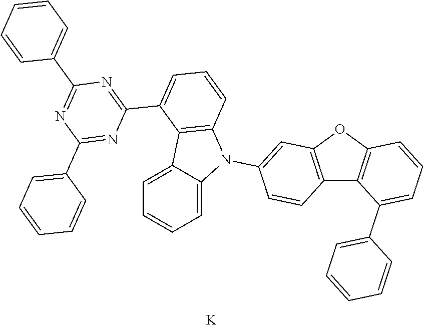

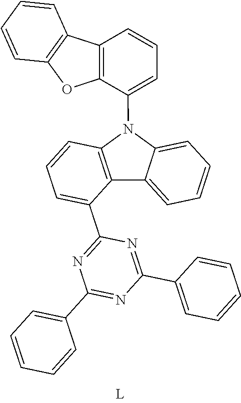 [SMILES: c1ccc(-c2nc(-c3ccccc3)nc(-c3cccc4c3c3ccccc3n4-c3cccc4c3oc3ccccc34)n2)cc1]

In the results of Table 10, Compounds A and B used in Examples 60 and 61 have structures in which a triazine group bonds to No. 2 position of carbazole, and No. 2 and No. 4 positions of dibenzofuran are each substituted with nitrogen of the carbazole. This is structurally similar to the compounds of Examples 14 and 16, however, high driving voltage, and low efficiency and lifetime were obtained. When a triazine group is introduced to the corresponding positions of Compounds A and B, a high T1 level is obtained, which may be advantageous in terms of efficiency, however, No. 2 and No. 4 positions of dibenzofuran are the same as cases of respectively substituting para and ortho positions of oxygen with carbazole. These positions are good positions at which degradation by radicalization may occur in the dibenzofuran, and a lifetime tended to rapidly decrease when substituted with an acceptor that attracts electrons since radicalization proceeded more readily. Such an effect was identified to be greater when a triazine group bonds to No. 2 position of carbazole.

Compounds C, D and F used in Comparative Examples 1, 2 and 4 are types in which an aryl group is not introduced to the ends of dibenzofuran and dibenzothiophene, and since the HOMO level region did not expand more widely, hole mobility increased and charge balance was affected, resulting in increased driving voltage and reduced efficiency. On the other hand, it was identified that, by introducing an aryl group to dibenzofuran, the compound of the present disclosure was capable of properly controlling hole mobility, stabilizing charge balance, and improving driving voltage and efficiency.

Compound E used in Comparative Example 3 has a structure substituted with dimethylfluorene, a cyclic group that does not include a heteroatom. In this structure, a low voltage and a similar level of efficiency were obtained, however, a low lifetime was obtained due to the weak bonding force at the position where methyl of the dimethylfluorene bonds. Dibenzofuran substituted with oxygen at the same position and thereby having a strong resonance structure has increased structural stability, and thereby has properties of longer lifetime.

Compound G used in Comparative Example 5 is a type having large steric hindrance due to the substituent bonding to the carbazole nitrogen. In this case, it was shown that structural stability decreased since structural distortion was intensified due to the steric hindrance. Both the HOMO and the LUMO levels were not able to spread widely and remained narrow within the substituent, and an unstable form was obtained since the orbital did not expand to the carbazole at the center, resulting in low efficiency and lifetime.

Compound H used in Comparative Example 6 has a structure in which positions of both benzene rings of carbazole are each substituted with triazine and dibenzofuran, which is different from the present disclosure, and has a bulkier structure by introducing an additional heteroaryl substituent. Such a structure has an effect of relatively increasing a driving voltage, and may act as a disadvantage when manufacturing a device.

Compounds I to L used in Comparative Examples 7 to 10 have structures in which a triazine group bonds to No. 4 position of carbazole, and, although structurally similar to the compound of the present disclosure, had a relatively low T1 value due to the bonding position of the substituent, and it was identified that this acted as a disadvantage of efficiency decrease when manufacturing a device.

Through such results, it is seen that devices properties may vary even with compounds having similar structures depending on the type of substituents and the position of substitution. This seems to be due to the fact that, when compounds are different, differences occur in the properties thereof, and properties of the compounds act as a major factor in enhancing device performance when depositing the compounds in a device manufacturing process.

Examining Examples 31 to 59 that are materials substituted with deuterium, an effect of further improving a lifetime was identified compared to Examples 1 to 30 that are materials not substituted with deuterium. A compound bonding with hydrogen and a compound substituted with deuterium are generally different in thermodynamic behavior. Such a reason is due to the fact that the mass of deuterium atom is twice the hydrogen, and by the difference in the atomic mass, deuterium tends to have lower vibration energy. In addition, a bond length between carbon and deuterium is shorter than a bond with hydrogen, and dissociation energy used to break the bond is also stronger. Such a reason is due to the fact that deuterium has a smaller Van der Waals radius compared to hydrogen, resulting in a narrower elongation amplitude of the bond between carbon-deuterium.

A compound substituted with deuterium has lower ground state energy than a compound substituted with hydrogen, and as the bond length between carbon-deuterium decreases, a molecular hardcore volume decreases. Electronical polarizability may be reduced therefrom, and, by further weakening intermolecular interactions, a device thin film volume may be increased. Such properties create an amorphous state of the thin film and induce an effect of lowering crystallinity. As a result, substitution with deuterium may be effective in enhancing heat resistance of an OLED device, which may improve lifetime properties of the device. In addition, the effect of enhancing device properties obtained from the substitution with deuterium is improved as the deuterium substitution ratio in the molecule increases.

### Experimental Example 2

### Experimental Example 2-1. Manufacture of Organic Light Emitting Device

A glass substrate coated with indium tin oxide (ITO) as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and then subjected to UVO (ultraviolet ozone) treatment for 5 minutes using UV (ultraviolet) in a UV cleaner. After that, the substrate was transferred to a plasma cleaner (PT), and after subjected to plasma treatment under vacuum for ITO work function increase and residual film removal, the substrate was transferred to a thermal deposition apparatus for organic deposition.

On the transparent ITO electrode (positive electrode), a hole injection layer 2-TNATA (4,4′,4″-tris[2-naphthyl(phenyl)amino]triphenylamine) was formed to 100 Å and a hole transport layer NPB (N,N′-di(1-naphthyl)-N,N′-diphenyl-(1,1′-biphenyl)-4,4′-diamine) was formed to 1100 Å as common layers.

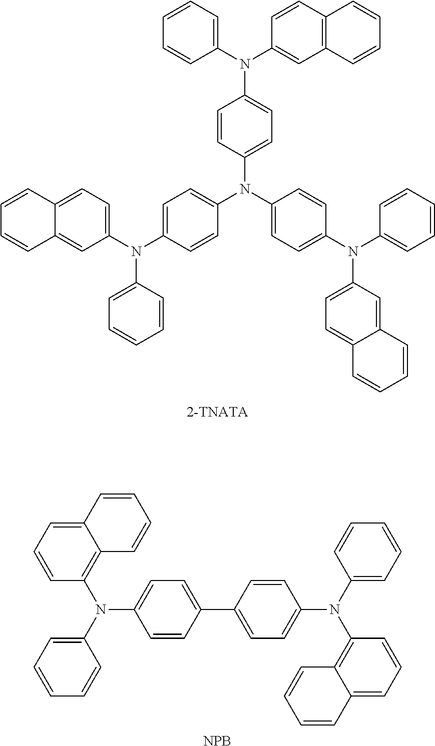 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

A light emitting layer was thermal vacuum deposited thereon as follows. As the light emitting layer, one type of a compound of Chemical Formula 1 and two types of compounds of Chemical Formula 2 described in the following Table 11 were pre-mixed and then deposited in one source of supply to a thickness of 400 Å as a host, and the host was doped with a green phosphorescent dopant Ir(ppy)₃ by 7% by weight with respect to the host weight. After that, BCP was deposited to a thickness of 60 Å as a hole blocking layer, and Alq₃ was deposited to a thickness of 200 Å thereon as an electron transport layer. Lastly, an electron injection layer was formed on the electron transport layer by depositing lithium fluoride (LiF) to a thickness of 10 Å, and then a negative electrode was formed on the electron injection layer by depositing aluminum (Al) to a thickness of 1,200 Å, and as a result, an organic light emitting device was manufactured.

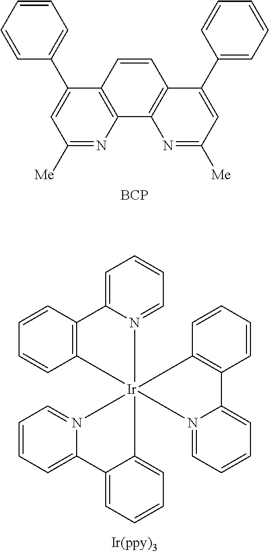

Meanwhile, all the organic compounds required to manufacture the OLED device were vacuum sublimation purified under 10-8 torr to 10-6 torr for each material to be used in the OLED manufacture.

### Experimental Example 2-2. Driving Voltage and Light Emission Efficiency of Organic Light Emitting Device

For each of the organic light emitting devices of Examples 62 to 125 and Comparative Examples 11 to 21 manufactured as above, electroluminescent (EL) properties were measured using M7000 manufactured by McScience Inc., and with the measurement results, T90 was measured when standard luminance was 6,000 cd/m² through a lifetime measurement system (M6000) manufactured by McScience Inc.

Properties of the organic electroluminescent devices of the present disclosure are as shown in the following Table 11. The T90 means a lifetime (unit: h, hour), a time taken for luminance to become 90% with respect to initial luminance.

TABLE 11

| | | | | Driving | | Color | Life |
|---|---|---|---|---|---|---|---|
| | | | | Voltage | Efficiency | Coordinate | time |
| | Compound 1 | Compound 2 | Ratio | (V) | (cd/A) | (x, y) | (T₉₀) |
| Comparative | C | 2-4 | 1:1 | 4.88 | 73.4 | (0.272, 0.675) | 328 |
| Example 11 | | | | | | | |
| Comparative | D | 2-4 | 1:1 | 4.85 | 74.2 | (0.272, 0.676) | 336 |
| Example 12 | | | | | | | |
| Comparative | E | 2-5 | 1:1 | 4.62 | 69.4 | (0.273, 0.681) | 317 |
| Example 13 | | | | | | | |
| Comparative | F | 2-7 | 1:1 | 4.97 | 71.5 | (0.276, 0.672) | 321 |
| Example 14 | | | | | | | |
| Comparative | G | 2-3 | 1:2 | 5.01 | 68.2 | (0.274, 0.671) | 227 |
| Example 15 | | | | | | | |
| Comparative | G | 2-3 | 1:1 | 4.89 | 72.3 | (0.275, 0.673) | 215 |
| Example 16 | | | | | | | |
| Comparative | H | 2-4 | 1:1 | 4.83 | 73.5 | (0.264, 0.675) | 318 |
| Example 17 | | | | | | | |
| Comparative | I | 2-3 | 1:1 | 4.45 | 76.2 | (0.277, 0.675) | 348 |
| Example 18 | | | | | | | |
| Comparative | J | 2-4 | 1:1 | 4.43 | 74.6 | (0.280, 0.673) | 335 |
| Example 19 | | | | | | | |
| Comparative | K | 2-5 | 1:1 | 4.39 | 75.1 | (0.272, 0.681) | 346 |
| Example 20 | | | | | | | |
| Comparative | L | 2-7 | 1:1 | 4.48 | 73.9 | (0.270, 0.675) | 339 |
| Example 21 | | | | | | | |
| Example 62 | 1-20 | 2-3 | 1:8 | 4.11 | 101.8 | (0.277, 0.674) | 453 |
| Example 63 | 1-20 | 2-3 | 1:5 | 3.62 | 102.4 | (0.275, 0.673) | 454 |
| Example 64 | 1-20 | 2-3 | 1:2 | 3.33 | 105.8 | (0.272, 0.674) | 468 |
| Example 65 | 1-20 | 2-3 | 1:1 | 3.22 | 107.3 | (0.280, 0.671) | 462 |
| Example 66 | 1-20 | 2-3 | 2:1 | 3.34 | 105.7 | (0.273, 0.675) | 460 |
| Example 67 | 1-20 | 2-3 | 5:1 | 3.40 | 104.2 | (0.275, 0.677) | 457 |
| Example 68 | 1-20 | 2-3 | 8:1 | 4.21 | 108.4 | (0.274, 0.679) | 453 |
| Example 69 | 1-68 | 2-4 | 1:2 | 3.34 | 105.1 | (0.272, 0.673) | 466 |
| Example 70 | 1-68 | 2-4 | 1:1 | 3.32 | 106.8 | (0.271, 0.672) | 462 |
| Example 71 | 1-92 | 2-5 | 1:2 | 3.32 | 106.2 | (0.273, 0.678) | 470 |
| Example 72 | 1-92 | 2-5 | 1:1 | 3.21 | 108.1 | (0.270, 0.679) | 465 |
| Example 73 | 1-131 | 2-7 | 1:2 | 3.35 | 105.2 | (0.281, 0.671) | 465 |
| Example 74 | 1-131 | 2-7 | 1:1 | 3.33 | 106.1 | (0.280, 0.674) | 463 |
| Example 75 | 1-156 | 2-31 | 1:2 | 3.38 | 102.8 | (0.271, 0.674) | 459 |
| Example 76 | 1-156 | 2-31 | 1:1 | 3.31 | 103.3 | (0.272, 0.677) | 454 |
| Example 77 | 1-430 | 2-32 | 1:2 | 3.36 | 103.2 | (0.272, 0.674) | 461 |
| Example 78 | 1-430 | 2-32 | 1:1 | 3.30 | 104.5 | (0.275, 0.672) | 455 |
| Example 79 | 1-182 | 2-42 | 1:2 | 3.30 | 110.6 | (0.281, 0.680) | 473 |
| Example 80 | 1-182 | 2-42 | 1:1 | 3.21 | 112.3 | (0.281, 0.683) | 467 |
| Example 81 | 1-207 | 2-3 | 1:2 | 3.31 | 110.8 | (0.280, 0.675) | 471 |
| Example 82 | 1-207 | 2-3 | 1:1 | 3.28 | 111.3 | (0.277, 0.672) | 467 |
| Example 83 | 1-240 | 2-4 | 1:2 | 3.28 | 111.6 | (0.281, 0.680) | 475 |
| Example 84 | 1-240 | 2-4 | 1:1 | 3.19 | 113.3 | (0.271, 0.682) | 470 |
| Example 85 | 1-276 | 2-7 | 1:2 | 3.32 | 110.4 | (0.273, 0.678) | 471 |
| Example 86 | 1-276 | 2-7 | 1:1 | 3.26 | 111.2 | (0.272, 0.677) | 468 |
| Example 87 | 1-20 | 2-108 | 1:2 | 3.37 | 120.6 | (0.275, 0.680) | 508 |
| Example 88 | 1-20 | 2-108 | 1:1 | 3.31 | 121.3 | (0.273, 0.682) | 506 |
| Example 89 | 1-20 | 2-115 | 1:2 | 3.26 | 123.7 | (0.275, 0.683) | 527 |
| Example 90 | 1-20 | 2-115 | 1:1 | 3.22 | 125.4 | (0.274, 0.681) | 524 |
| Example 91 | 1-410 | 2-3 | 1:2 | 3.39 | 121.2 | (0.282, 0.677) | 505 |
| Example 92 | 1-410 | 2-3 | 1:1 | 3.31 | 125.4 | (0.281, 0.671) | 502 |
| Example 93 | 1-410 | 2-108 | 1:2 | 3.12 | 131.2 | (0.280, 0.674) | 529 |
| Example 94 | 1-410 | 2-108 | 1:1 | 3.10 | 132.3 | (0.282, 0.676) | 526 |
| Example 95 | 1-410 | 2-115 | 1:2 | 3.15 | 131.9 | (0.276, 0.677) | 541 |
| Example 96 | 1-410 | 2-115 | 1:1 | 3.11 | 132.5 | (0.276, 0.679) | 538 |
| Example 97 | 1-416 | 2-3 | 1:2 | 3.23 | 122.4 | (0.274, 0.675) | 503 |
| Example 98 | 1-416 | 2-3 | 1:1 | 3.20 | 125.6 | (0.272, 0.673) | 500 |
| Example 99 | 1-416 | 2-108 | 1:2 | 3.16 | 131.3 | (0.282, 0.679) | 538 |
| Example 100 | 1-416 | 2-108 | 1:1 | 3.12 | 132.4 | (0.284, 0.676) | 532 |
| Example 101 | 1-416 | 2-115 | 1:2 | 3.03 | 135.3 | (0.273, 0.674) | 554 |
| Example 102 | 1-416 | 2-115 | 1:1 | 2.97 | 137.6 | (0.272, 0.673) | 552 |
| Example 103 | 1-312 | 2-3 | 1:2 | 3.24 | 121.7 | (0.275, 0.675) | 505 |
| Example 104 | 1-312 | 2-3 | 1:1 | 3.21 | 124.2 | (0.272, 0.673) | 500 |
| Example 105 | 1-328 | 2-4 | 1:2 | 3.38 | 120.8 | (0.281, 0.685) | 501 |
| Example 106 | 1-328 | 2-4 | 1:1 | 3.31 | 123.3 | (0.280, 0.678) | 498 |
| Example 107 | 1-344 | 2-5 | 1:2 | 3.23 | 122.4 | (0.281, 0.674) | 507 |
| Example 108 | 1-344 | 2-5 | 1:1 | 3.20 | 125.6 | (0.280, 0.672) | 501 |
| Example 109 | 1-356 | 2-7 | 1:2 | 3.37 | 120.5 | (0.281, 0.683) | 500 |
| Example 110 | 1-356 | 2-7 | 1:1 | 3.32 | 123.8 | (0.273, 0.682) | 497 |
| Example 111 | 1-372 | 2-31 | 1:2 | 3.39 | 118.4 | (0.275, 0.678) | 502 |
| Example 112 | 1-372 | 2-31 | 1:1 | 3.34 | 121.5 | (0.276, 0.676) | 498 |
| Example 113 | 1-384 | 2-32 | 1:2 | 3.38 | 119.8 | (0.273, 0.682) | 504 |
| Example 114 | 1-384 | 2-32 | 1:1 | 3.33 | 121.9 | (0.274, 0.684) | 500 |
| Example 115 | 1-392 | 2-4 | 1:2 | 3.22 | 130.8 | (0.276, 0.682) | 515 |
| Example 116 | 1-392 | 2-4 | 1:1 | 3.18 | 131.6 | (0.274, 0.681) | 510 |
| Example 117 | 1-404 | 2-5 | 1:2 | 3.24 | 129.4 | (0.273, 0.677) | 508 |
| Example 118 | 1-404 | 2-5 | 1:1 | 3.22 | 130.9 | (0.275, 0.677) | 505 |
| Example 119 | 1-416 | 2-7 | 1:2 | 3.23 | 131.7 | (0.273, 0.682) | 514 |
| Example 120 | 1-416 | 2-7 | 1:1 | 3.19 | 132.4 | (0.275, 0.682) | 510 |
| Example 121 | 1-428 | 2-31 | 1:2 | 3.25 | 130.1 | (0.280, 0.683) | 506 |
| Example 122 | 1-428 | 2-31 | 1:1 | 3.23 | 131.7 | (0.281, 0.682) | 504 |
| Example 123 | A | 2-3 | 1:2 | 4.67 | 77.2 | (0.281, 0.682) | 394 |
| Example 124 | A | 2-3 | 1:1 | 4.37 | 82.7 | (0.278, 0.676) | 385 |
| Example 125 | B | 2-3 | 1:1 | 4.42 | 81.2 | (0.282, 0.684) | 379 |

[Compounds A to L]

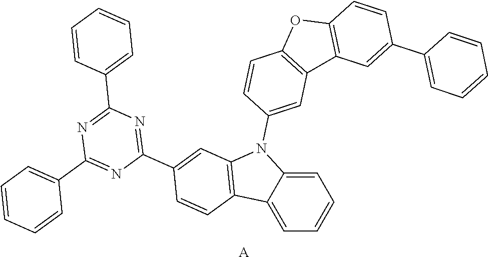

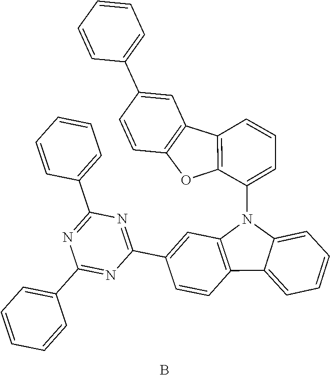

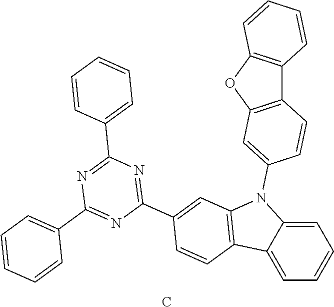

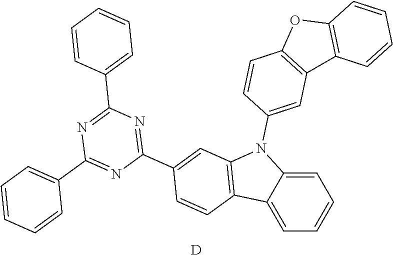

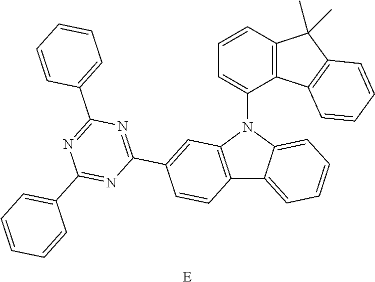

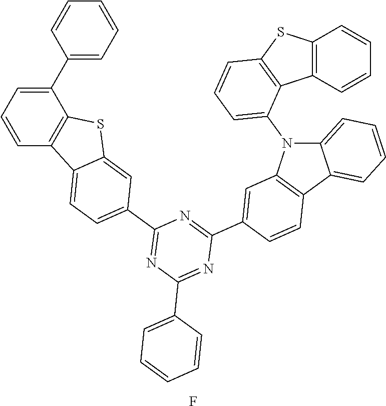

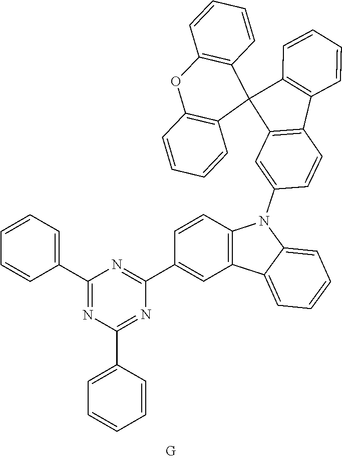 [SMILES: c1ccc(-c2nc(-c3ccccc3)nc(-c3ccc4c(c3)c3ccccc3n4-c3ccc4c(c3)C3(c5ccccc5Oc5ccccc53)c3ccccc3-4)n2)cc1]

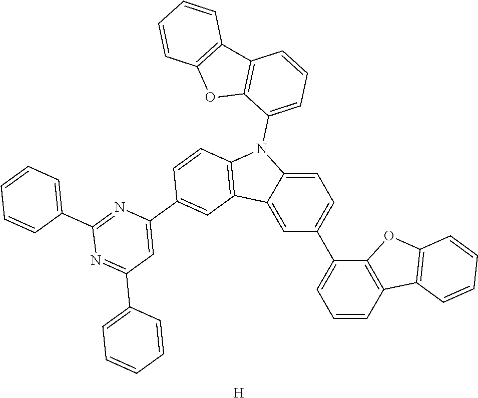

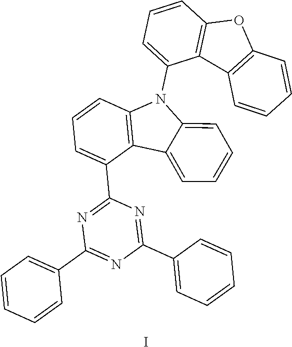

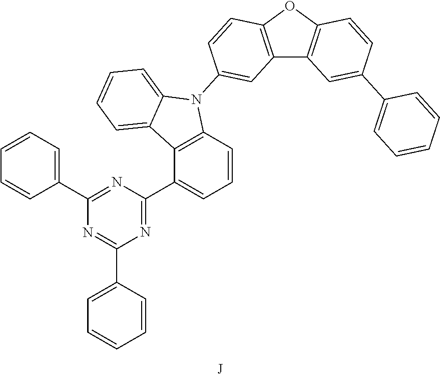

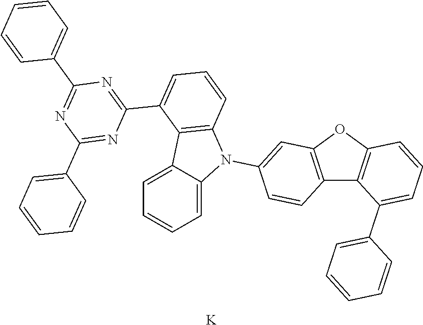

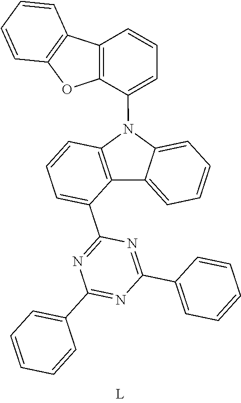 [SMILES: c1ccc(-c2nc(-c3ccccc3)nc(-c3cccc4c3c3ccccc3n4-c3cccc4c3oc3ccccc34)n2)cc1]

From the results of Table 11, effects of superior efficiency and lifetime were obtained when using the compound represented by Chemical Formula 1 (n-type) according to the present disclosure with the compound represented by Chemical Formula 2 (p-type). This is due to the fact that an exciplex phenomenon occurs when including the two compounds at the same time.

The exciplex phenomenon is a phenomenon of releasing energy having sizes of a HOMO energy level of a donor (p-host) and a LUMO energy level of an acceptor (n-host) due to electron exchanges between two molecules. When the exciplex phenomenon occurs between two molecules, reverse intersystem crossing (RISC) occurs, and as a result, internal quantum efficiency of fluorescence emission may increase up to 100%. When a donor (p-host) having a favorable hole transport ability and an acceptor (n-host) having a favorable electron transport ability are used as a host of a light emitting layer, holes are injected to the p-host and electrons are injected to the n-host, and herein, excitons are not quenched due to the electron exchange between molecules, and a lifetime of the excitons capable of having energy increases. This improves overall current efficiency, and in addition thereto, may help with enhancement in the device lifetime. In the present disclosure, it was identified that superior device properties were obtained when, as the light emitting layer host, using the compound of Chemical Formula 2 as a donor role and the compound of Chemical Formula 1 as an acceptor role.

The pre-mixing according to the present disclosure means, before depositing the heterocyclic compound of Chemical Formula 1 and the heterocyclic compound of Chemical Formula 2 on the organic material layer, mixing the materials first in one source of supply. Since one deposition source is used instead of two or three deposition sources in the pre-mixing, there is an advantage of making the process simpler.

During pre-mixing as described above, unique thermal properties of each material need to be checked before mixing. Herein, when depositing the pre-mixed host material from one deposition source, unique thermal properties of each of the materials may greatly affect deposition conditions including a deposition rate. When thermal properties between two or more types of the pre-mixed materials are not similar and differ greatly, repeatability and reproducibility in the deposition process may not be maintained, and this means that an OLED device that is all uniform may not be manufactured in one deposition process.

By using a proper combination of the basic structure and substituents of each material in order to overcome this, thermal properties of the material may also be controlled depending on the molecular structure type while tuning electrical properties of the material. Therefore, diversity of various pre-mixing deposition processes between host-host may be secured by controlling thermal properties of each material as well as attempting to enhance device performance by using, as well as the C—N bond of carbazole as in Chemical Formula 2, various substituents in the triazine group or carbazole/dibenzofuran of Chemical Formula 1 in addition to the basic skeleton. This has an advantage of securing diversity of pre-mixing deposition processes using, as a host, three, four or more host materials as well as two compounds.

As the mixing of the heterocyclic compound of Chemical Formula 1 and the heterocyclic compound of Chemical Formula 2, two or more types (three, four types and the like) of materials may be mixed, and the experimental examples are only representative examples, and mixing is not limited thereto.

Through the mixing of the heterocyclic compound of Chemical Formula 1 and the heterocyclic compound of Chemical Formula 2, an effect of partially improving current efficiency of the light emitting layer may be obtained, and a device having long lifetime properties may be obtained as well.

From the results of Table 11, effects of superior efficiency and lifetime were obtained when the heterocyclic compound of Chemical Formula 1 and the heterocyclic compound of Chemical Formula 2 are included at the same time. This is due to the exciplex phenomenon occurring when two or more materials are mixed.

A driving voltage increases in some cases when the exciplex phenomenon occurs. This is due to the fact that an imbalance occurs between holes and electrons in a light emitting layer of a device. This is a problem caused by deviations in hole and electron mobilities of each material between mixed hosts. Therefore, a device exhibiting optimal performance may be obtained when properly maintaining a balance in the electron flow in the device. The corresponding problem was able to be resolved through adjusting a ratio between the acceptor and the donor.

### REFERENCE NUMERAL

100: Substrate
200: Positive Electrode
300: Organic Material Layer
301: Hole Injection Layer
302: Hole Transport Layer
303: Light Emitting Layer
304: Hole Blocking Layer
305: Electron Transport Layer
306: Electron Injection Layer
400: Negative Electrode

1. A heterocyclic compound represented by the following Chemical Formula 1: 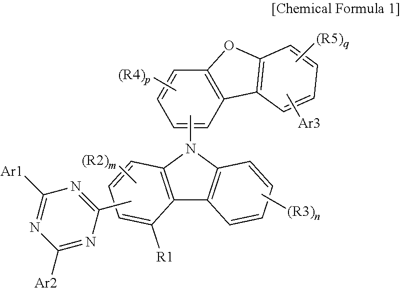
wherein, in Chemical Formula 1,
R1 is hydrogen; or deuterium;
R2 to R5 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
Ar1 to Ar3 are the same as or different from each other, and each independently a substituted or unsubstituted C6 to C60 aryl group; or a substituted or unsubstituted C2 to C60 heteroaryl group;
m is an integer of 0 to 2, and when m is 2 or greater, R2s are the same as or different from each other;
n is an integer of 0 to 4, and when n is 2 or greater, R3s are the same as or different from each other;
p is an integer of 0 to 3, and when p is 2 or greater, R4s are the same as or different from each other; and
q is an integer of 0 to 3, and when q is 2 or greater, R5s are the same as or different from each other.

2. The heterocyclic compound of claim 1, wherein the heterocyclic compound represented by Chemical Formula 1 is represented by any one of the following Chemical Formulae 1-1 to 1-4: 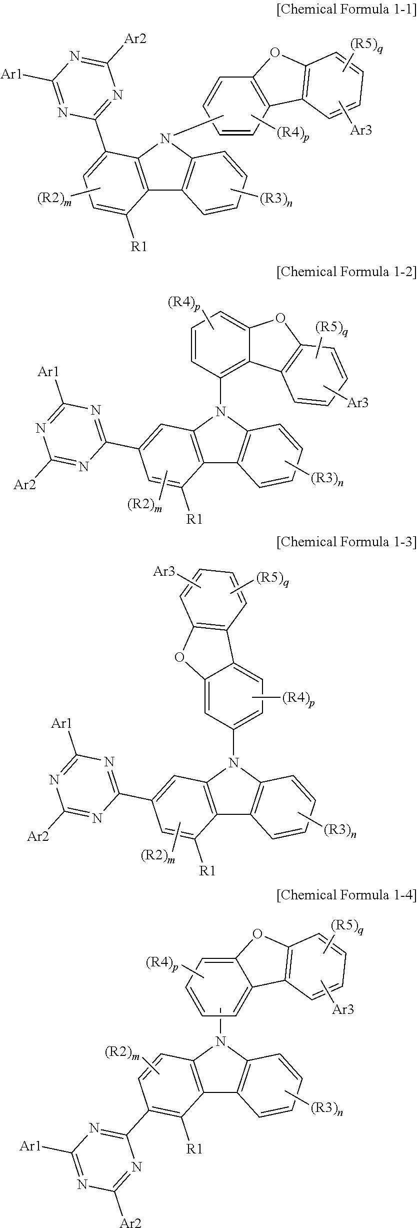
in Chemical Formulae 1-1 to 1-4,
R1 to R5, Ar1 to Ar3, m, n, p and q have the same definitions as in Chemical Formula 1.

3. The heterocyclic compound of claim 1, wherein Ar3 is a substituted or unsubstituted C6 to C60 aryl group.

4. The heterocyclic compound of claim 1, wherein the heterocyclic compound represented by Chemical Formula 1 does not include deuterium as a substituent, or has a deuterium content of 1% to 100% with respect to a total number of hydrogen atoms and deuterium atoms.

5. The heterocyclic compound of claim 1, wherein the heterocyclic compound represented by Chemical Formula 1 is represented by any one of the following compounds:

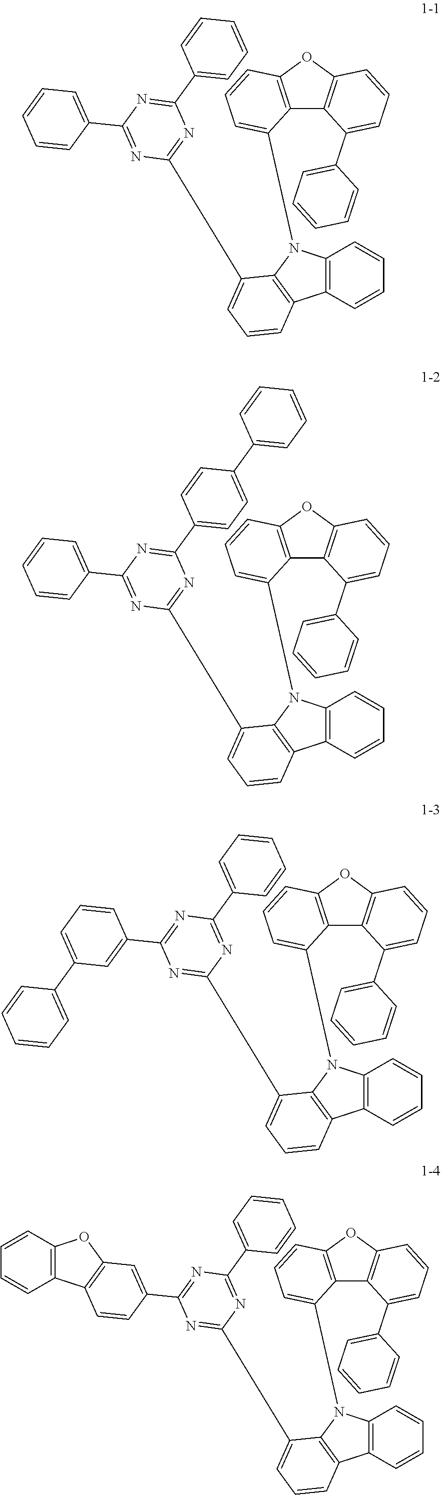

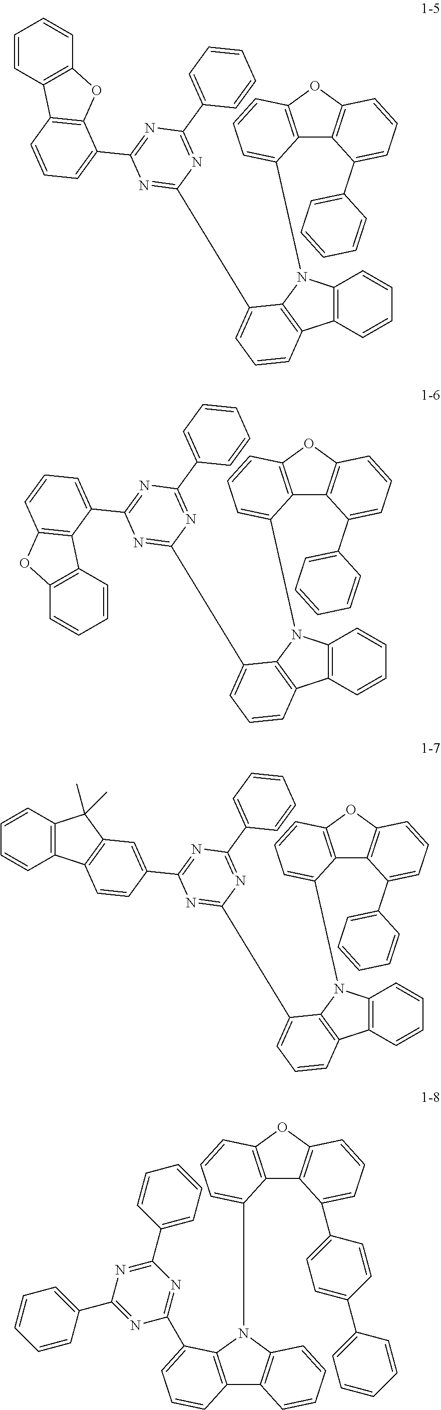 [SMILES: c1ccc(-c2nc(-c3cccc4c3oc3ccccc34)nc(-c3cccc4c5ccccc5n(-c5cccc6oc7cccc(-c8ccccc8)c7c56)c34)n2)cc1 ; c1ccc(-c2nc(-c3cccc4oc5ccccc5c34)nc(-c3cccc4c5ccccc5n(-c5cccc6oc7cccc(-c8ccccc8)c7c56)c34)n2)cc1 ; CC1(C)c2ccccc2-c2ccc(-c3nc(-c4ccccc4)nc(-c4cccc5c6ccccc6n(-c6cccc7oc8cccc(-c9ccccc9)c8c67)c45)n3)cc21 ; c1ccc(-c2ccc(-c3cccc4oc5cccc(-n6c7ccccc7c7cccc(-c8nc(-c9ccccc9)nc(-c9ccccc9)n8)c76)c5c34)cc2)cc1]

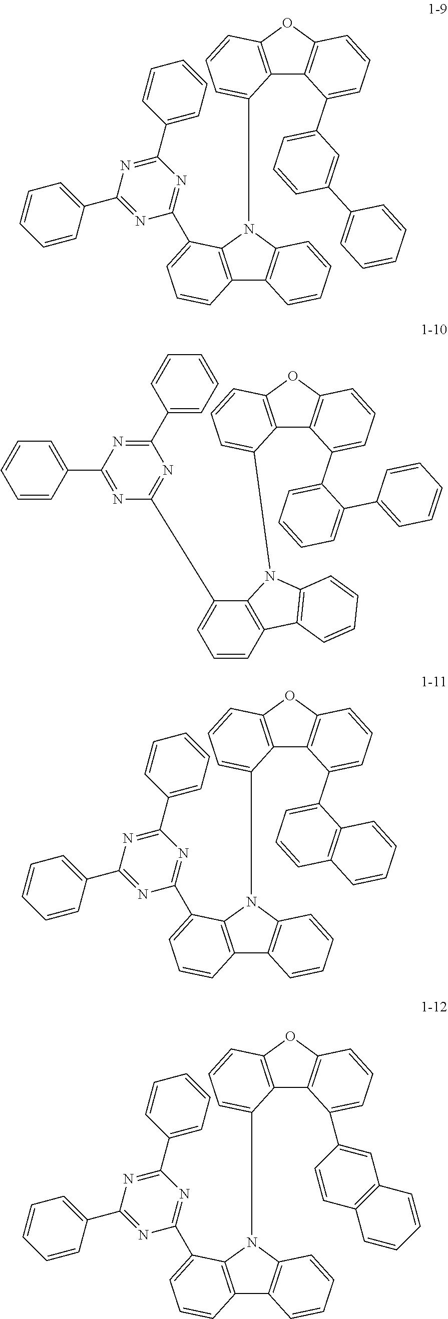 [SMILES: c1ccc(-c2cccc(-c3cccc4oc5cccc(-n6c7ccccc7c7cccc(-c8nc(-c9ccccc9)nc(-c9ccccc9)n8)c76)c5c34)c2)cc1 ; c1ccc(-c2nc(-c3ccccc3)nc(-c3cccc4c5ccccc5n(-c5cccc6oc7cccc(-c8ccccc8-c8ccccc8)c7c56)c34)n2)cc1 ; c1ccc(-c2nc(-c3ccccc3)nc(-c3cccc4c5ccccc5n(-c5cccc6oc7cccc(-c8cccc9ccccc89)c7c56)c34)n2)cc1 ; c1ccc(-c2nc(-c3ccccc3)nc(-c3cccc4c5ccccc5n(-c5cccc6oc7cccc(-c8ccc9ccccc9c8)c7c56)c34)n2)cc1]

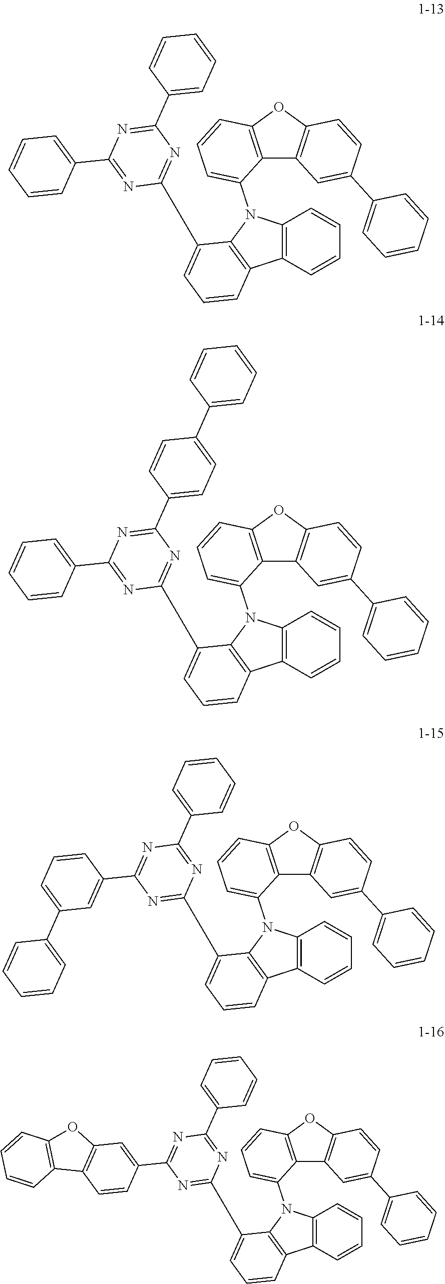 [SMILES: c1ccc(-c2ccc3oc4cccc(-n5c6ccccc6c6cccc(-c7nc(-c8ccccc8)nc(-c8ccccc8)n7)c65)c4c3c2)cc1 ; c1ccc(-c2ccc(-c3nc(-c4ccccc4)nc(-c4cccc5c6ccccc6n(-c6cccc7oc8ccc(-c9ccccc9)cc8c67)c45)n3)cc2)cc1 ; c1ccc(-c2cccc(-c3nc(-c4ccccc4)nc(-c4cccc5c6ccccc6n(-c6cccc7oc8ccc(-c9ccccc9)cc8c67)c45)n3)c2)cc1 ; c1ccc(-c2ccc3oc4cccc(-n5c6ccccc6c6cccc(-c7nc(-c8ccccc8)nc(-c8ccc9c(c8)oc8ccccc89)n7)c65)c4c3c2)cc1]

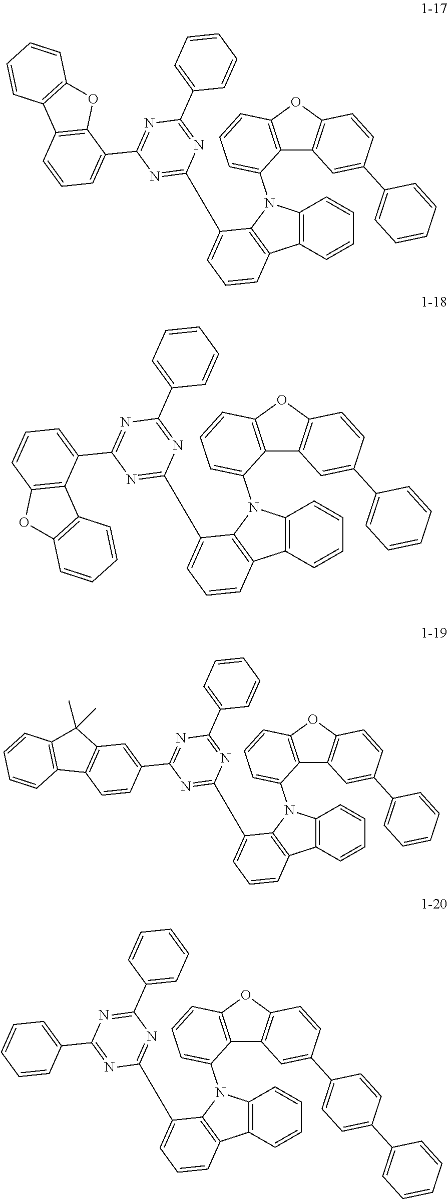 [SMILES: c1ccc(-c2ccc3oc4cccc(-n5c6ccccc6c6cccc(-c7nc(-c8ccccc8)nc(-c8cccc9c8oc8ccccc89)n7)c65)c4c3c2)cc1 ; c1ccc(-c2ccc3oc4cccc(-n5c6ccccc6c6cccc(-c7nc(-c8ccccc8)nc(-c8cccc9oc%10ccccc%10c89)n7)c65)c4c3c2)cc1 ; CC1(C)c2ccccc2-c2ccc(-c3nc(-c4ccccc4)nc(-c4cccc5c6ccccc6n(-c6cccc7oc8ccc(-c9ccccc9)cc8c67)c45)n3)cc21 ; c1ccc(-c2ccc(-c3ccc4oc5cccc(-n6c7ccccc7c7cccc(-c8nc(-c9ccccc9)nc(-c9ccccc9)n8)c76)c5c4c3)cc2)cc1]

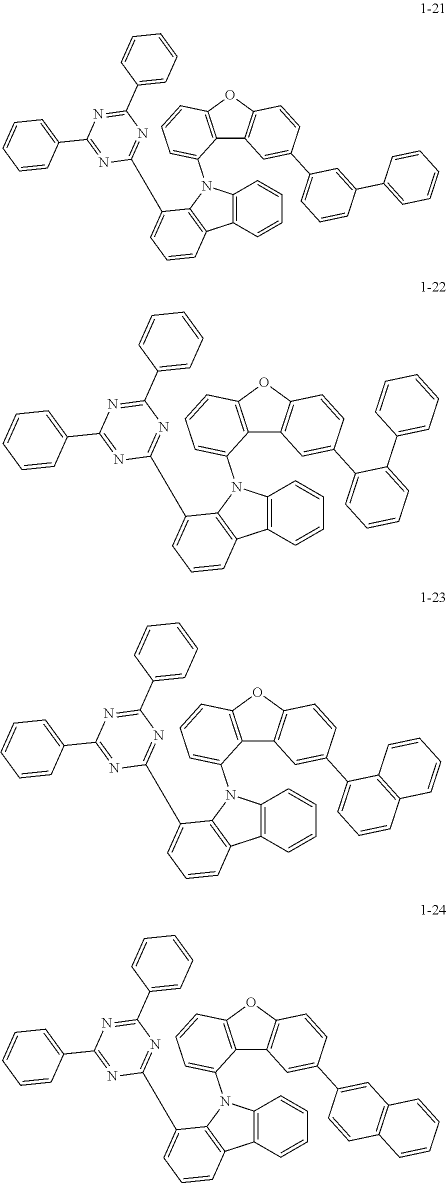 [SMILES: c1ccc(-c2cccc(-c3ccc4oc5cccc(-n6c7ccccc7c7cccc(-c8nc(-c9ccccc9)nc(-c9ccccc9)n8)c76)c5c4c3)c2)cc1 ; c1ccc(-c2nc(-c3ccccc3)nc(-c3cccc4c5ccccc5n(-c5cccc6oc7ccc(-c8ccccc8-c8ccccc8)cc7c56)c34)n2)cc1 ; c1ccc(-c2nc(-c3ccccc3)nc(-c3cccc4c5ccccc5n(-c5cccc6oc7ccc(-c8cccc9ccccc89)cc7c56)c34)n2)cc1 ; c1ccc(-c2nc(-c3ccccc3)nc(-c3cccc4c5ccccc5n(-c5cccc6oc7ccc(-c8ccc9ccccc9c8)cc7c56)c34)n2)cc1]

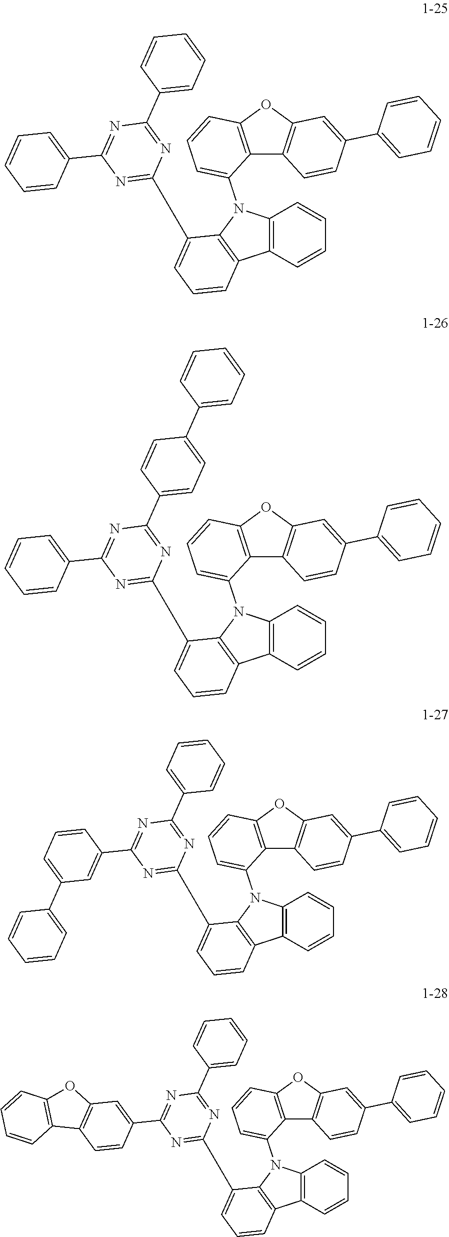 [SMILES: c1ccc(-c2ccc3c(c2)oc2cccc(-n4c5ccccc5c5cccc(-c6nc(-c7ccccc7)nc(-c7ccccc7)n6)c54)c23)cc1 ; c1ccc(-c2ccc(-c3nc(-c4ccccc4)nc(-c4cccc5c6ccccc6n(-c6cccc7oc8cc(-c9ccccc9)ccc8c67)c45)n3)cc2)cc1 ; c1ccc(-c2cccc(-c3nc(-c4ccccc4)nc(-c4cccc5c6ccccc6n(-c6cccc7oc8cc(-c9ccccc9)ccc8c67)c45)n3)c2)cc1 ; c1ccc(-c2ccc3c(c2)oc2cccc(-n4c5ccccc5c5cccc(-c6nc(-c7ccccc7)nc(-c7ccc8c(c7)oc7ccccc78)n6)c54)c23)cc1]

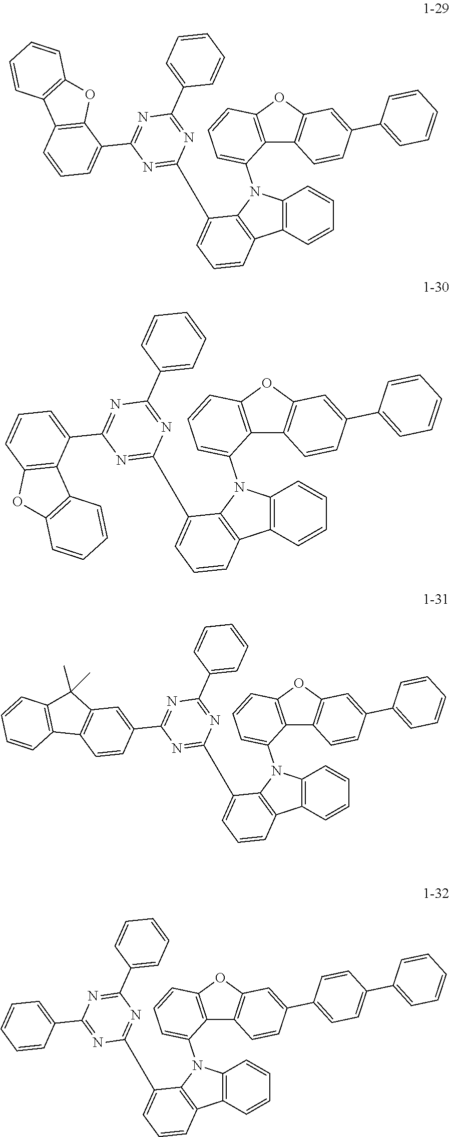 [SMILES: c1ccc(-c2ccc3c(c2)oc2cccc(-n4c5ccccc5c5cccc(-c6nc(-c7ccccc7)nc(-c7cccc8c7oc7ccccc78)n6)c54)c23)cc1 ; c1ccc(-c2ccc3c(c2)oc2cccc(-n4c5ccccc5c5cccc(-c6nc(-c7ccccc7)nc(-c7cccc8oc9ccccc9c78)n6)c54)c23)cc1 ; CC1(C)c2ccccc2-c2ccc(-c3nc(-c4ccccc4)nc(-c4cccc5c6ccccc6n(-c6cccc7oc8cc(-c9ccccc9)ccc8c67)c45)n3)cc21 ; c1ccc(-c2ccc(-c3ccc4c(c3)oc3cccc(-n5c6ccccc6c6cccc(-c7nc(-c8ccccc8)nc(-c8ccccc8)n7)c65)c34)cc2)cc1]

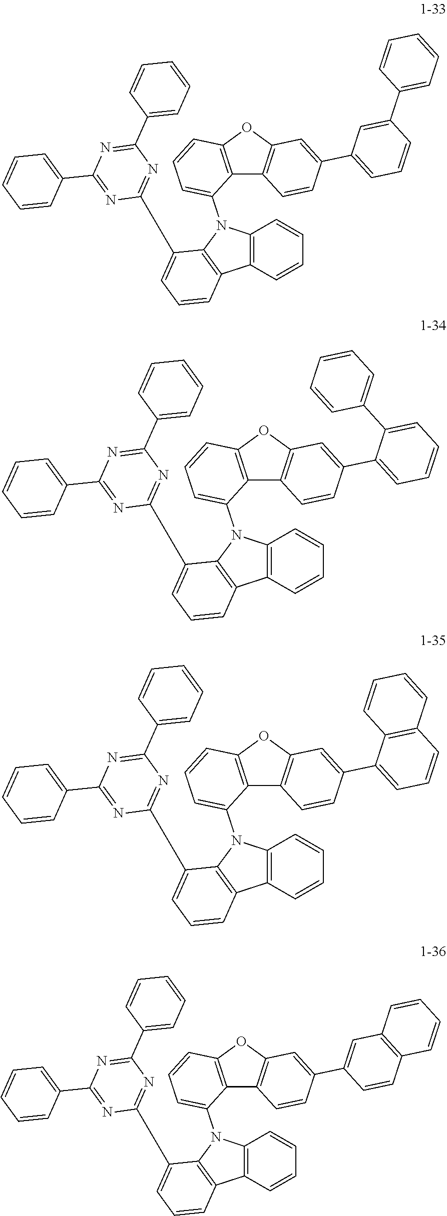 [SMILES: c1ccc(-c2cccc(-c3ccc4c(c3)oc3cccc(-n5c6ccccc6c6cccc(-c7nc(-c8ccccc8)nc(-c8ccccc8)n7)c65)c34)c2)cc1 ; c1ccc(-c2nc(-c3ccccc3)nc(-c3cccc4c5ccccc5n(-c5cccc6oc7cc(-c8ccccc8-c8ccccc8)ccc7c56)c34)n2)cc1 ; c1ccc(-c2nc(-c3ccccc3)nc(-c3cccc4c5ccccc5n(-c5cccc6oc7cc(-c8cccc9ccccc89)ccc7c56)c34)n2)cc1 ; c1ccc(-c2nc(-c3ccccc3)nc(-c3cccc4c5ccccc5n(-c5cccc6oc7cc(-c8ccc9ccccc9c8)ccc7c56)c34)n2)cc1]

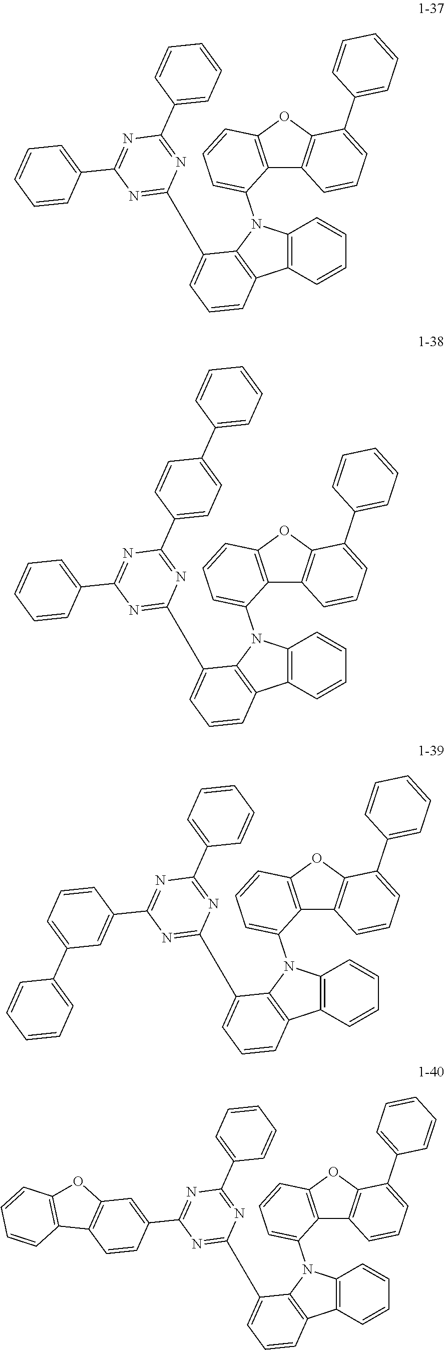 [SMILES: c1ccc(-c2nc(-c3ccccc3)nc(-c3cccc4c5ccccc5n(-c5cccc6oc7c(-c8ccccc8)cccc7c56)c34)n2)cc1 ; c1ccc(-c2ccc(-c3nc(-c4ccccc4)nc(-c4cccc5c6ccccc6n(-c6cccc7oc8c(-c9ccccc9)cccc8c67)c45)n3)cc2)cc1 ; c1ccc(-c2cccc(-c3nc(-c4ccccc4)nc(-c4cccc5c6ccccc6n(-c6cccc7oc8c(-c9ccccc9)cccc8c67)c45)n3)c2)cc1 ; c1ccc(-c2nc(-c3ccc4c(c3)oc3ccccc34)nc(-c3cccc4c5ccccc5n(-c5cccc6oc7c(-c8ccccc8)cccc7c56)c34)n2)cc1]

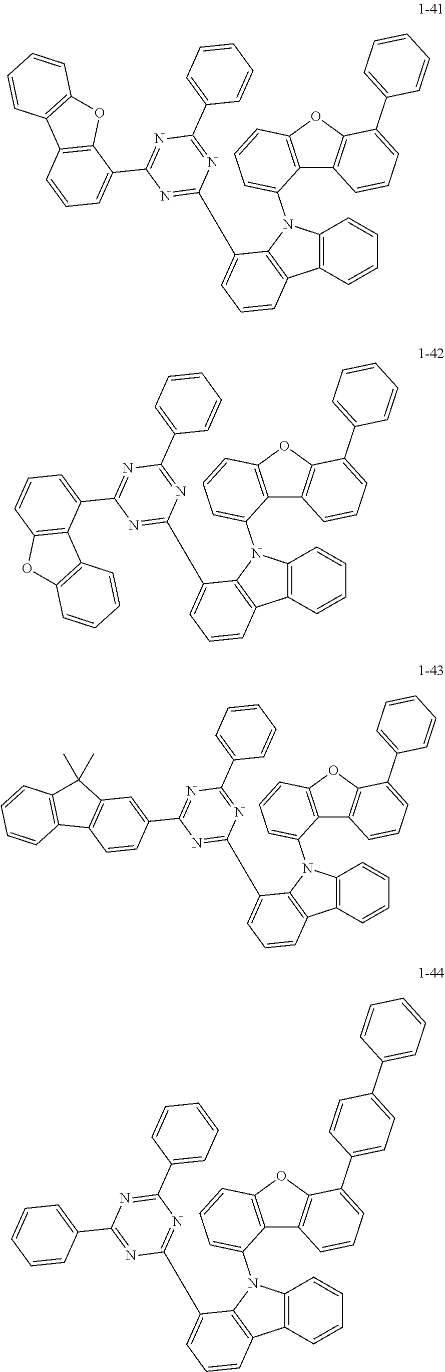 [SMILES: c1ccc(-c2nc(-c3cccc4c3oc3ccccc34)nc(-c3cccc4c5ccccc5n(-c5cccc6oc7c(-c8ccccc8)cccc7c56)c34)n2)cc1 ; c1ccc(-c2nc(-c3cccc4oc5ccccc5c34)nc(-c3cccc4c5ccccc5n(-c5cccc6oc7c(-c8ccccc8)cccc7c56)c34)n2)cc1 ; CC1(C)c2ccccc2-c2ccc(-c3nc(-c4ccccc4)nc(-c4cccc5c6ccccc6n(-c6cccc7oc8c(-c9ccccc9)cccc8c67)c45)n3)cc21 ; c1ccc(-c2ccc(-c3cccc4c3oc3cccc(-n5c6ccccc6c6cccc(-c7nc(-c8ccccc8)nc(-c8ccccc8)n7)c65)c34)cc2)cc1]

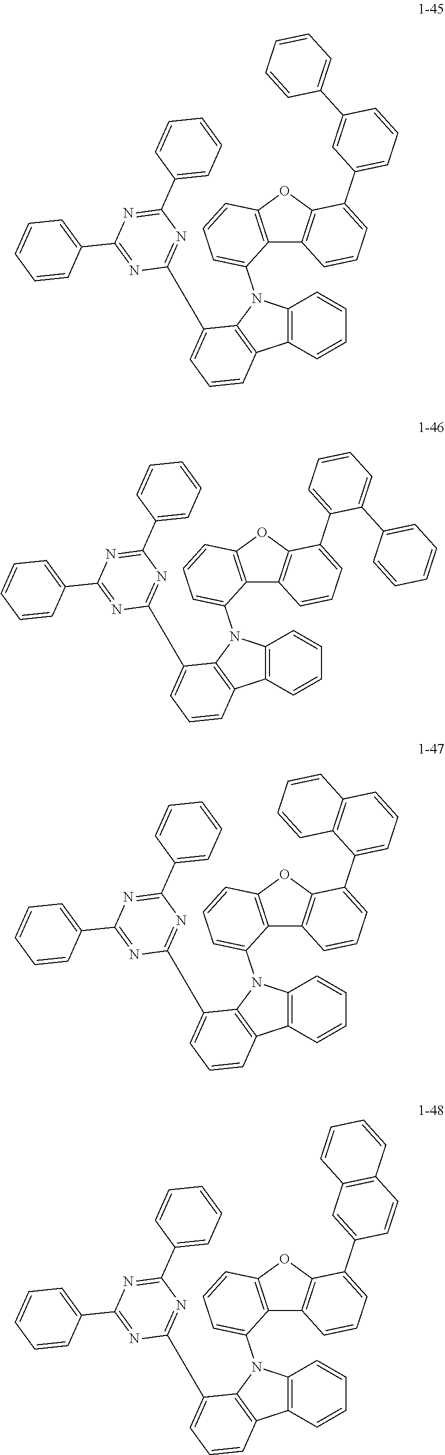 [SMILES: c1ccc(-c2cccc(-c3cccc4c3oc3cccc(-n5c6ccccc6c6cccc(-c7nc(-c8ccccc8)nc(-c8ccccc8)n7)c65)c34)c2)cc1 ; c1ccc(-c2nc(-c3ccccc3)nc(-c3cccc4c5ccccc5n(-c5cccc6oc7c(-c8ccccc8-c8ccccc8)cccc7c56)c34)n2)cc1 ; c1ccc(-c2nc(-c3ccccc3)nc(-c3cccc4c5ccccc5n(-c5cccc6oc7c(-c8cccc9ccccc89)cccc7c56)c34)n2)cc1 ; c1ccc(-c2nc(-c3ccccc3)nc(-c3cccc4c5ccccc5n(-c5cccc6oc7c(-c8ccc9ccccc9c8)cccc7c56)c34)n2)cc1]

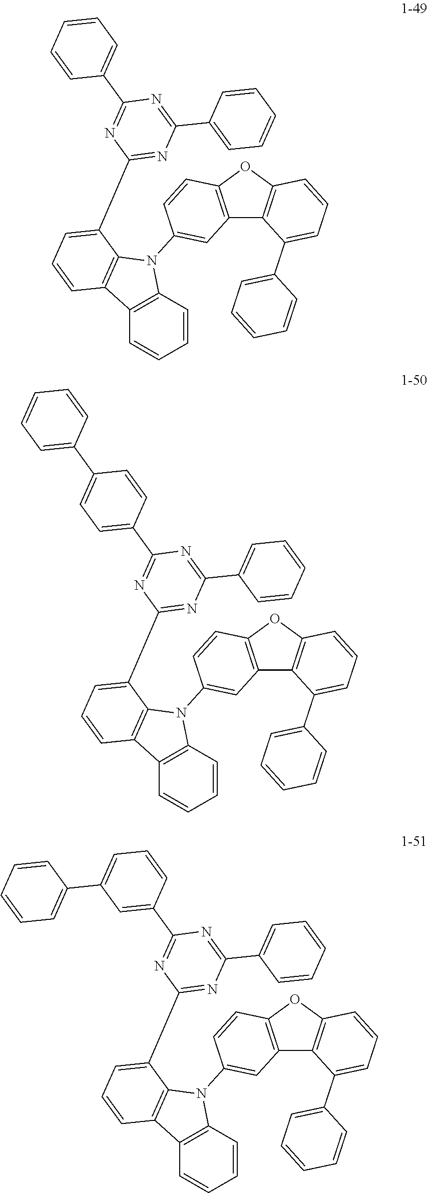 [SMILES: c1ccc(-c2nc(-c3ccccc3)nc(-c3cccc4c5ccccc5n(-c5ccc6oc7cccc(-c8ccccc8)c7c6c5)c34)n2)cc1 ; c1ccc(-c2ccc(-c3nc(-c4ccccc4)nc(-c4cccc5c6ccccc6n(-c6ccc7oc8cccc(-c9ccccc9)c8c7c6)c45)n3)cc2)cc1 ; c1ccc(-c2cccc(-c3nc(-c4ccccc4)nc(-c4cccc5c6ccccc6n(-c6ccc7oc8cccc(-c9ccccc9)c8c7c6)c45)n3)c2)cc1]

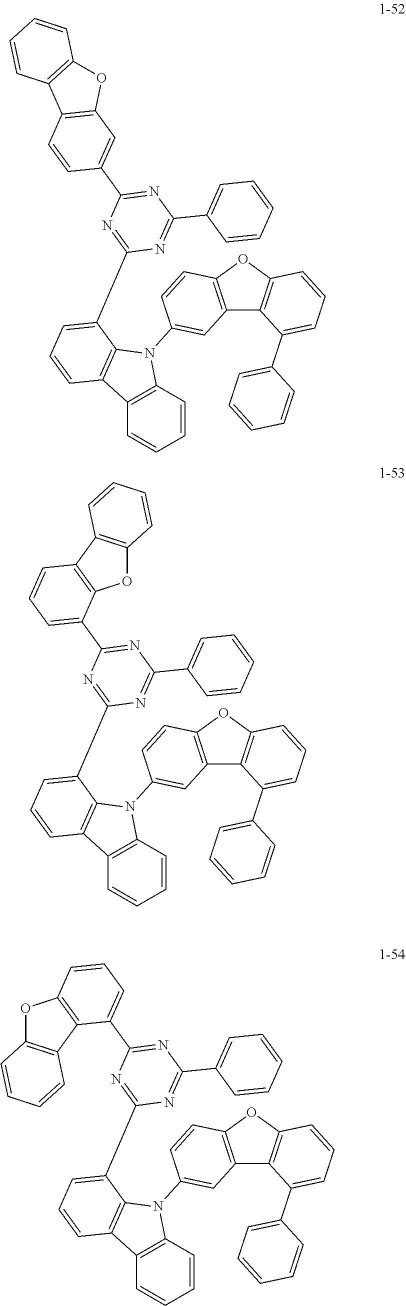

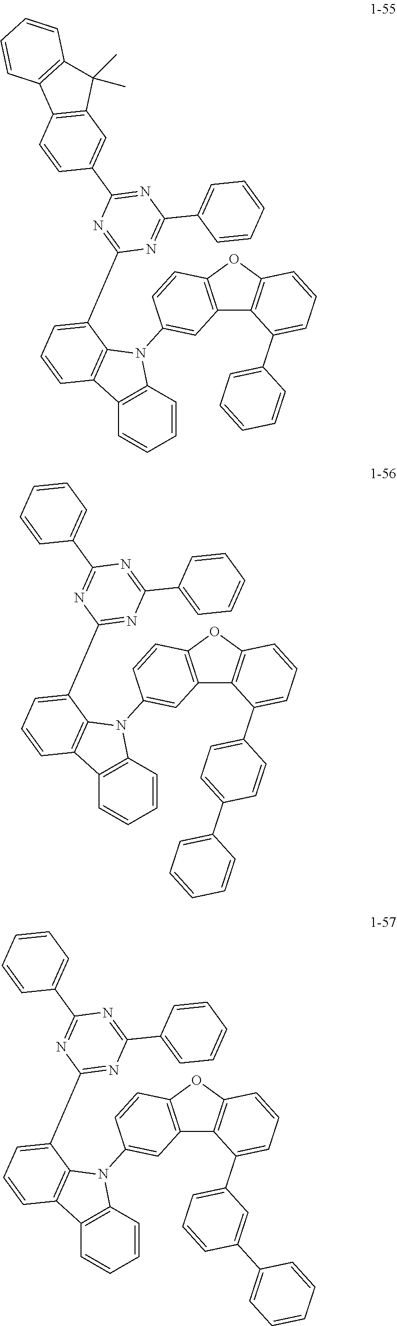 [SMILES: CC1(C)c2ccccc2-c2ccc(-c3nc(-c4ccccc4)nc(-c4cccc5c6ccccc6n(-c6ccc7oc8cccc(-c9ccccc9)c8c7c6)c45)n3)cc21 ; c1ccc(-c2ccc(-c3cccc4oc5ccc(-n6c7ccccc7c7cccc(-c8nc(-c9ccccc9)nc(-c9ccccc9)n8)c76)cc5c34)cc2)cc1 ; c1ccc(-c2cccc(-c3cccc4oc5ccc(-n6c7ccccc7c7cccc(-c8nc(-c9ccccc9)nc(-c9ccccc9)n8)c76)cc5c34)c2)cc1]

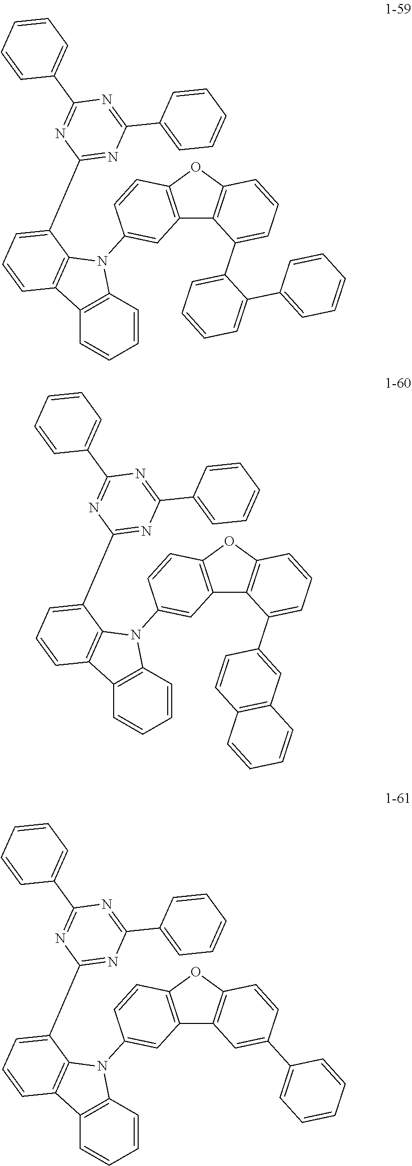

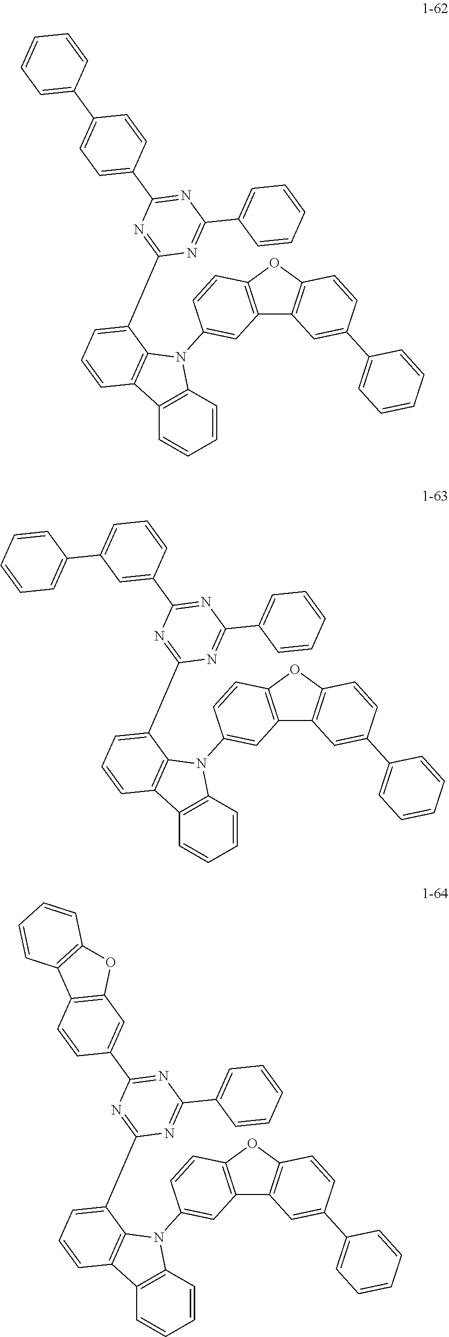 [SMILES: c1ccc(-c2ccc(-c3nc(-c4ccccc4)nc(-c4cccc5c6ccccc6n(-c6ccc7oc8ccc(-c9ccccc9)cc8c7c6)c45)n3)cc2)cc1 ; c1ccc(-c2cccc(-c3nc(-c4ccccc4)nc(-c4cccc5c6ccccc6n(-c6ccc7oc8ccc(-c9ccccc9)cc8c7c6)c45)n3)c2)cc1 ; c1ccc(-c2ccc3oc4ccc(-n5c6ccccc6c6cccc(-c7nc(-c8ccccc8)nc(-c8ccc9c(c8)oc8ccccc89)n7)c65)cc4c3c2)cc1]

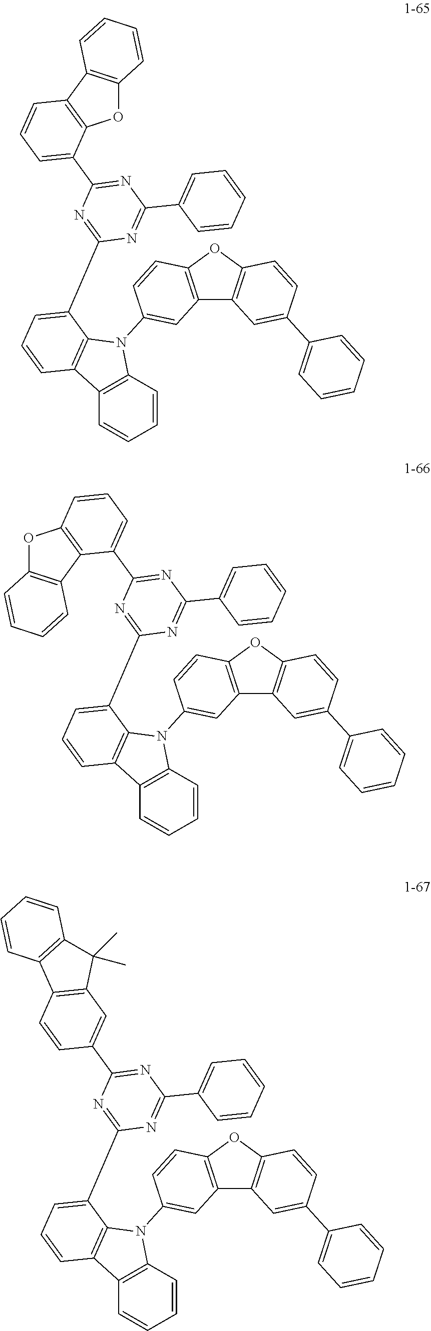 [SMILES: c1ccc(-c2ccc3oc4ccc(-n5c6ccccc6c6cccc(-c7nc(-c8ccccc8)nc(-c8cccc9c8oc8ccccc89)n7)c65)cc4c3c2)cc1 ; c1ccc(-c2ccc3oc4ccc(-n5c6ccccc6c6cccc(-c7nc(-c8ccccc8)nc(-c8cccc9oc%10ccccc%10c89)n7)c65)cc4c3c2)cc1 ; CC1(C)c2ccccc2-c2ccc(-c3nc(-c4ccccc4)nc(-c4cccc5c6ccccc6n(-c6ccc7oc8ccc(-c9ccccc9)cc8c7c6)c45)n3)cc21]

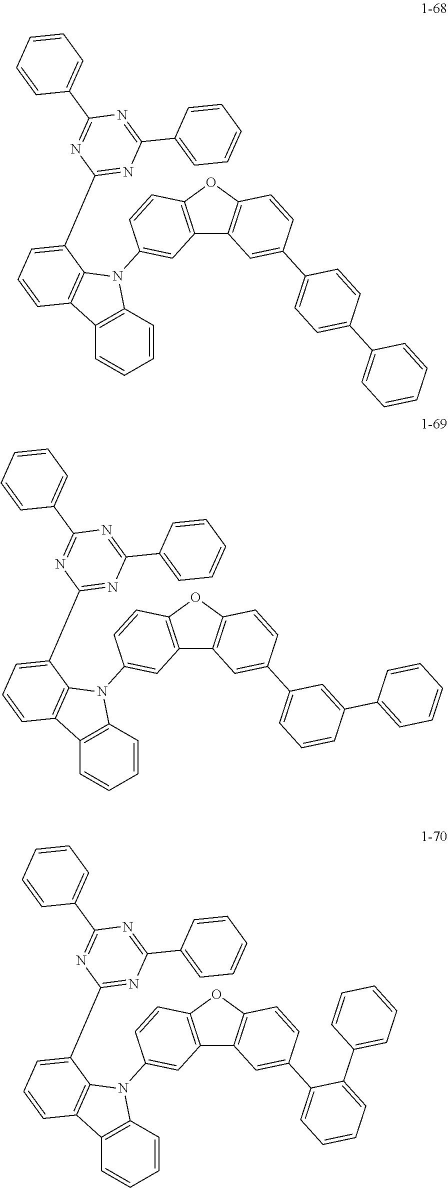 [SMILES: c1ccc(-c2ccc(-c3ccc4oc5ccc(-n6c7ccccc7c7cccc(-c8nc(-c9ccccc9)nc(-c9ccccc9)n8)c76)cc5c4c3)cc2)cc1 ; c1ccc(-c2cccc(-c3ccc4oc5ccc(-n6c7ccccc7c7cccc(-c8nc(-c9ccccc9)nc(-c9ccccc9)n8)c76)cc5c4c3)c2)cc1 ; c1ccc(-c2nc(-c3ccccc3)nc(-c3cccc4c5ccccc5n(-c5ccc6oc7ccc(-c8ccccc8-c8ccccc8)cc7c6c5)c34)n2)cc1]

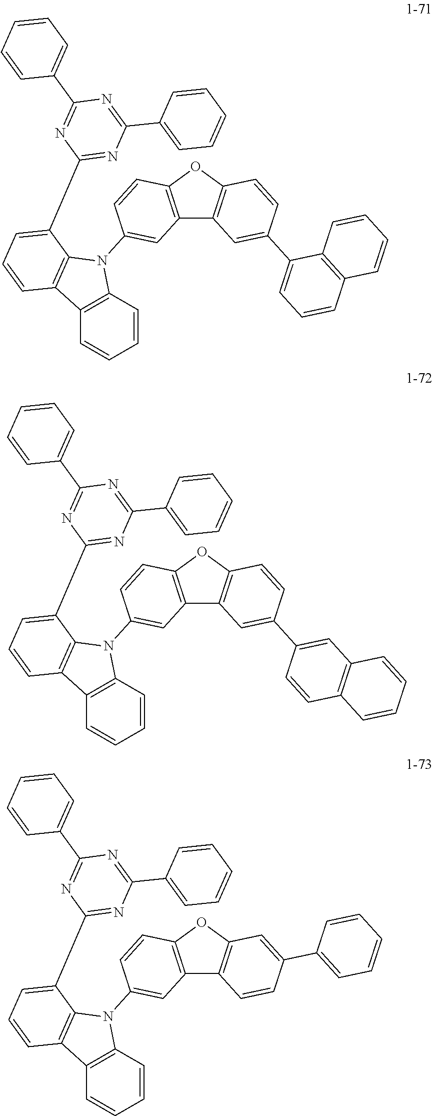 [SMILES: c1ccc(-c2nc(-c3ccccc3)nc(-c3cccc4c5ccccc5n(-c5ccc6oc7ccc(-c8cccc9ccccc89)cc7c6c5)c34)n2)cc1 ; c1ccc(-c2nc(-c3ccccc3)nc(-c3cccc4c5ccccc5n(-c5ccc6oc7ccc(-c8ccc9ccccc9c8)cc7c6c5)c34)n2)cc1 ; c1ccc(-c2ccc3c(c2)oc2ccc(-n4c5ccccc5c5cccc(-c6nc(-c7ccccc7)nc(-c7ccccc7)n6)c54)cc23)cc1]

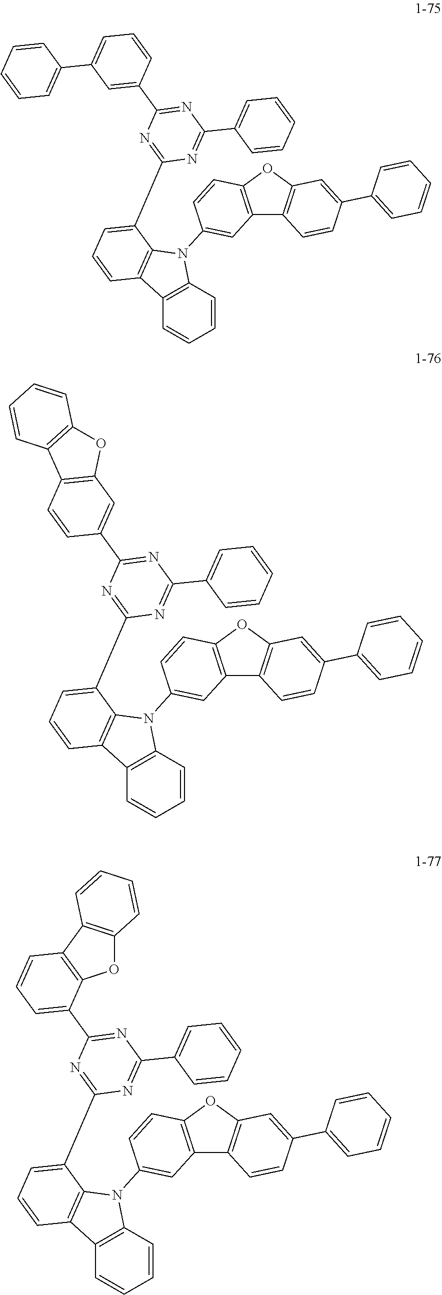 [SMILES: c1ccc(-c2cccc(-c3nc(-c4ccccc4)nc(-c4cccc5c6ccccc6n(-c6ccc7oc8cc(-c9ccccc9)ccc8c7c6)c45)n3)c2)cc1 ; c1ccc(-c2ccc3c(c2)oc2ccc(-n4c5ccccc5c5cccc(-c6nc(-c7ccccc7)nc(-c7ccc8c(c7)oc7ccccc78)n6)c54)cc23)cc1 ; c1ccc(-c2ccc3c(c2)oc2ccc(-n4c5ccccc5c5cccc(-c6nc(-c7ccccc7)nc(-c7cccc8c7oc7ccccc78)n6)c54)cc23)cc1]

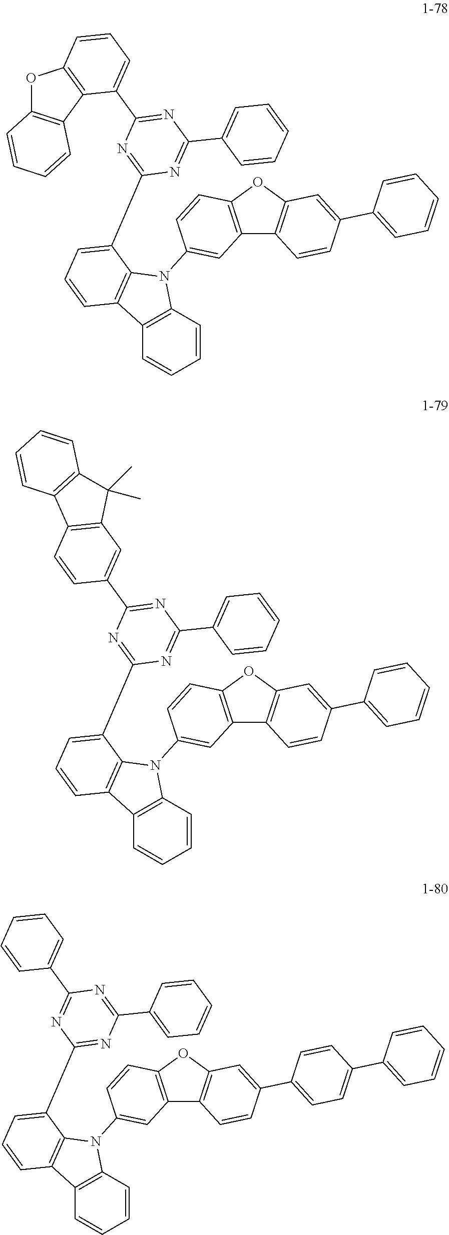 [SMILES: c1ccc(-c2ccc3c(c2)oc2ccc(-n4c5ccccc5c5cccc(-c6nc(-c7ccccc7)nc(-c7cccc8oc9ccccc9c78)n6)c54)cc23)cc1 ; CC1(C)c2ccccc2-c2ccc(-c3nc(-c4ccccc4)nc(-c4cccc5c6ccccc6n(-c6ccc7oc8cc(-c9ccccc9)ccc8c7c6)c45)n3)cc21 ; c1ccc(-c2ccc(-c3ccc4c(c3)oc3ccc(-n5c6ccccc6c6cccc(-c7nc(-c8ccccc8)nc(-c8ccccc8)n7)c65)cc34)cc2)cc1]

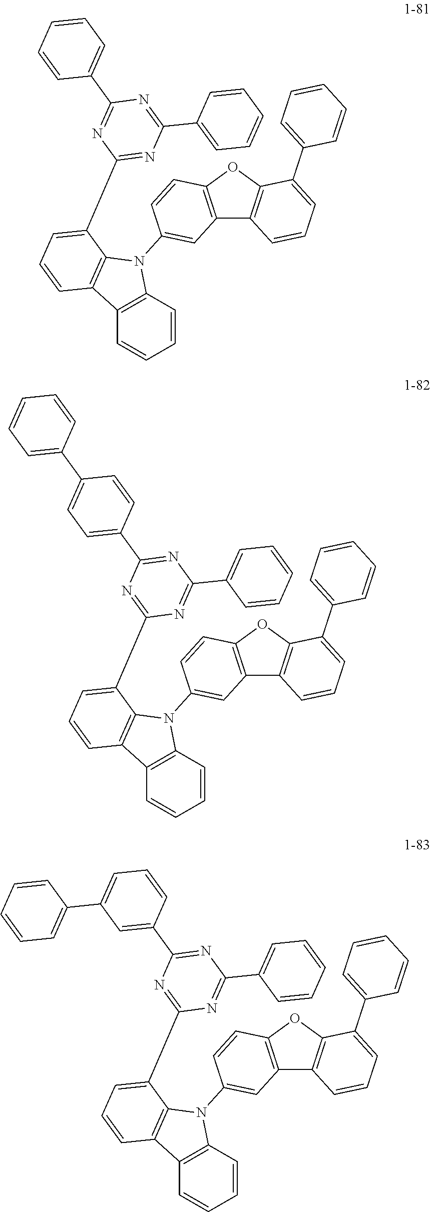 [SMILES: c1ccc(-c2nc(-c3ccccc3)nc(-c3cccc4c5ccccc5n(-c5ccc6oc7c(-c8ccccc8)cccc7c6c5)c34)n2)cc1 ; c1ccc(-c2ccc(-c3nc(-c4ccccc4)nc(-c4cccc5c6ccccc6n(-c6ccc7oc8c(-c9ccccc9)cccc8c7c6)c45)n3)cc2)cc1 ; c1ccc(-c2cccc(-c3nc(-c4ccccc4)nc(-c4cccc5c6ccccc6n(-c6ccc7oc8c(-c9ccccc9)cccc8c7c6)c45)n3)c2)cc1]

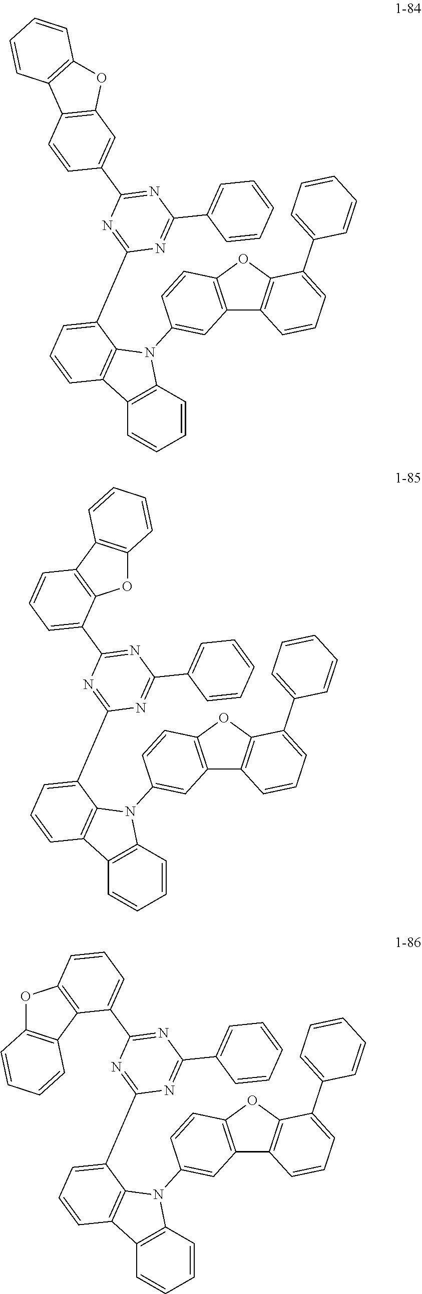 [SMILES: c1ccc(-c2nc(-c3ccc4c(c3)oc3ccccc34)nc(-c3cccc4c5ccccc5n(-c5ccc6oc7c(-c8ccccc8)cccc7c6c5)c34)n2)cc1 ; c1ccc(-c2nc(-c3cccc4c3oc3ccccc34)nc(-c3cccc4c5ccccc5n(-c5ccc6oc7c(-c8ccccc8)cccc7c6c5)c34)n2)cc1 ; c1ccc(-c2nc(-c3cccc4oc5ccccc5c34)nc(-c3cccc4c5ccccc5n(-c5ccc6oc7c(-c8ccccc8)cccc7c6c5)c34)n2)cc1]

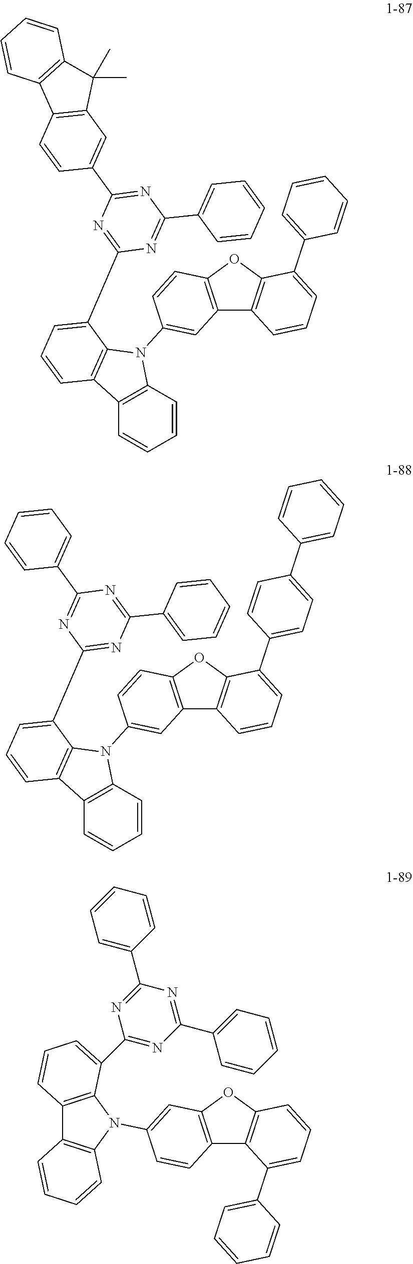

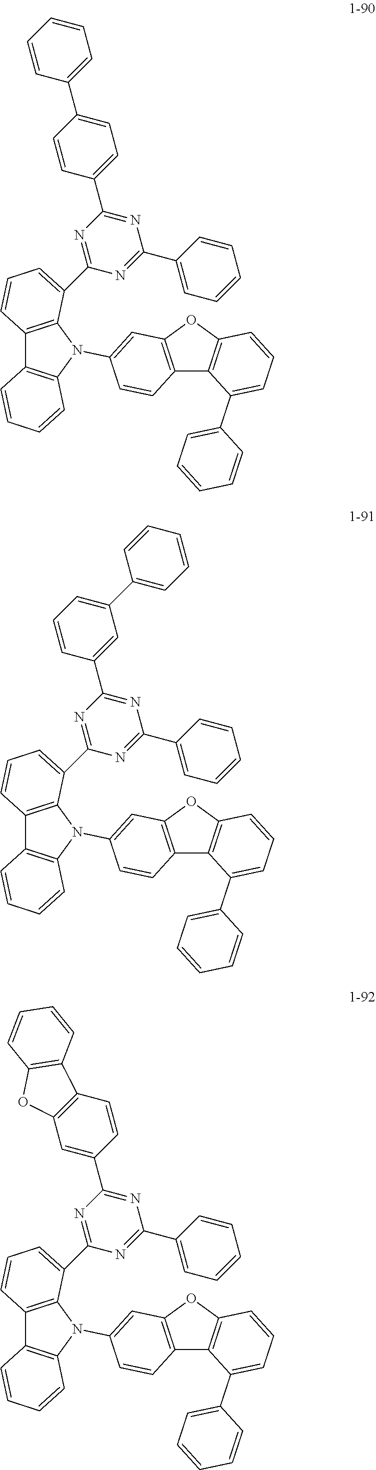 [SMILES: c1ccc(-c2ccc(-c3nc(-c4ccccc4)nc(-c4cccc5c6ccccc6n(-c6ccc7c(c6)oc6cccc(-c8ccccc8)c67)c45)n3)cc2)cc1 ; c1ccc(-c2cccc(-c3nc(-c4ccccc4)nc(-c4cccc5c6ccccc6n(-c6ccc7c(c6)oc6cccc(-c8ccccc8)c67)c45)n3)c2)cc1 ; c1ccc(-c2nc(-c3ccc4c(c3)oc3ccccc34)nc(-c3cccc4c5ccccc5n(-c5ccc6c(c5)oc5cccc(-c7ccccc7)c56)c34)n2)cc1]

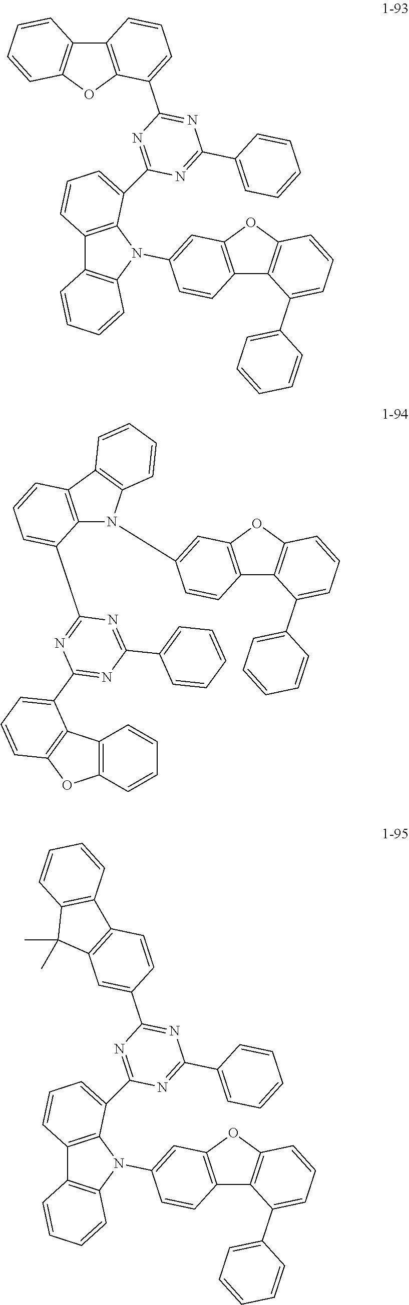 [SMILES: c1ccc(-c2nc(-c3cccc4c3oc3ccccc34)nc(-c3cccc4c5ccccc5n(-c5ccc6c(c5)oc5cccc(-c7ccccc7)c56)c34)n2)cc1 ; c1ccc(-c2nc(-c3cccc4oc5ccccc5c34)nc(-c3cccc4c5ccccc5n(-c5ccc6c(c5)oc5cccc(-c7ccccc7)c56)c34)n2)cc1 ; CC1(C)c2ccccc2-c2ccc(-c3nc(-c4ccccc4)nc(-c4cccc5c6ccccc6n(-c6ccc7c(c6)oc6cccc(-c8ccccc8)c67)c45)n3)cc21]

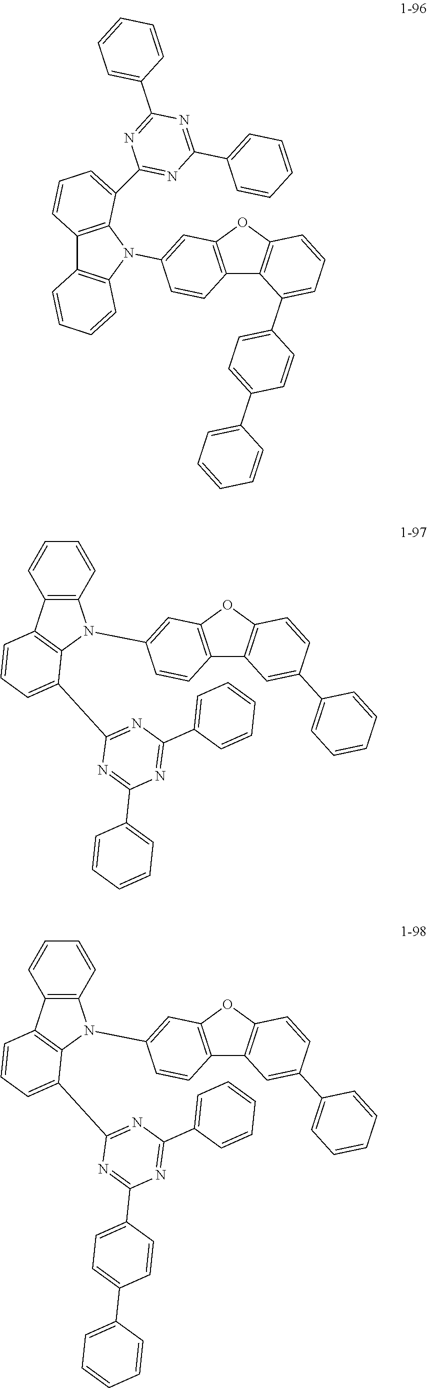 [SMILES: c1ccc(-c2ccc(-c3cccc4oc5cc(-n6c7ccccc7c7cccc(-c8nc(-c9ccccc9)nc(-c9ccccc9)n8)c76)ccc5c34)cc2)cc1 ; c1ccc(-c2ccc3oc4cc(-n5c6ccccc6c6cccc(-c7nc(-c8ccccc8)nc(-c8ccccc8)n7)c65)ccc4c3c2)cc1 ; c1ccc(-c2ccc(-c3nc(-c4ccccc4)nc(-c4cccc5c6ccccc6n(-c6ccc7c(c6)oc6ccc(-c8ccccc8)cc67)c45)n3)cc2)cc1]

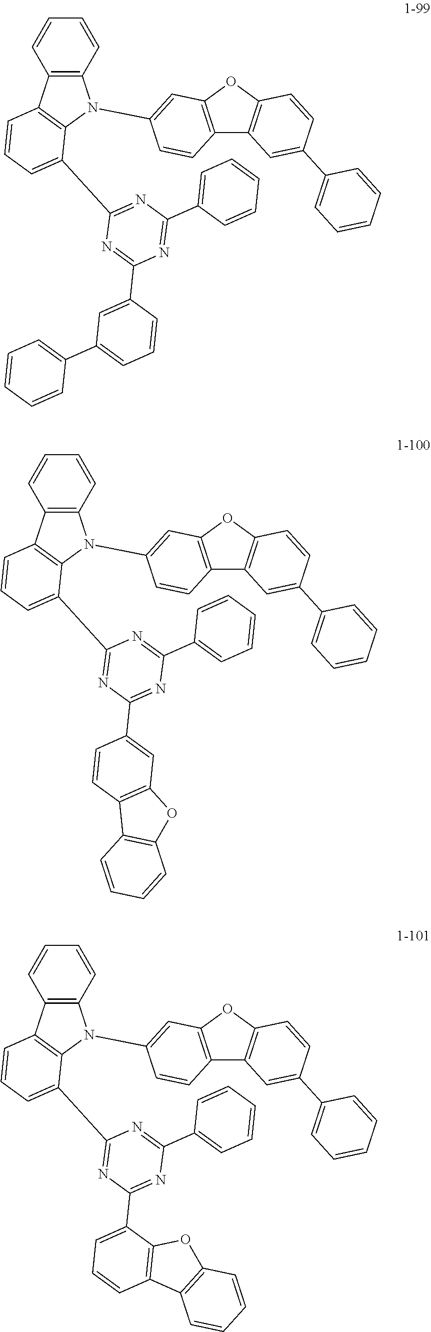 [SMILES: c1ccc(-c2cccc(-c3nc(-c4ccccc4)nc(-c4cccc5c6ccccc6n(-c6ccc7c(c6)oc6ccc(-c8ccccc8)cc67)c45)n3)c2)cc1 ; c1ccc(-c2ccc3oc4cc(-n5c6ccccc6c6cccc(-c7nc(-c8ccccc8)nc(-c8ccc9c(c8)oc8ccccc89)n7)c65)ccc4c3c2)cc1 ; c1ccc(-c2ccc3oc4cc(-n5c6ccccc6c6cccc(-c7nc(-c8ccccc8)nc(-c8cccc9c8oc8ccccc89)n7)c65)ccc4c3c2)cc1]

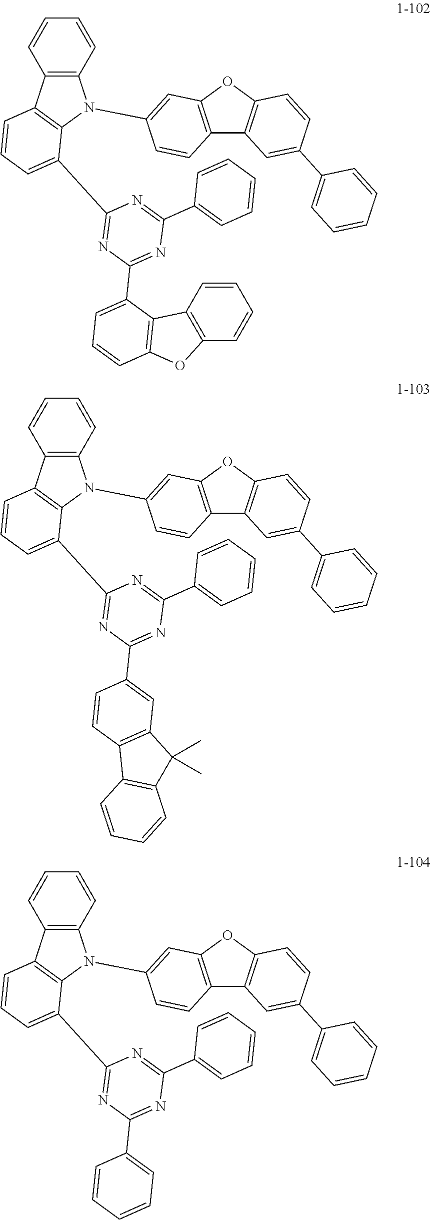 [SMILES: c1ccc(-c2ccc3oc4cc(-n5c6ccccc6c6cccc(-c7nc(-c8ccccc8)nc(-c8cccc9oc%10ccccc%10c89)n7)c65)ccc4c3c2)cc1 ; CC1(C)c2ccccc2-c2ccc(-c3nc(-c4ccccc4)nc(-c4cccc5c6ccccc6n(-c6ccc7c(c6)oc6ccc(-c8ccccc8)cc67)c45)n3)cc21 ; c1ccc(-c2ccc3oc4cc(-n5c6ccccc6c6cccc(-c7nc(-c8ccccc8)nc(-c8ccccc8)n7)c65)ccc4c3c2)cc1]

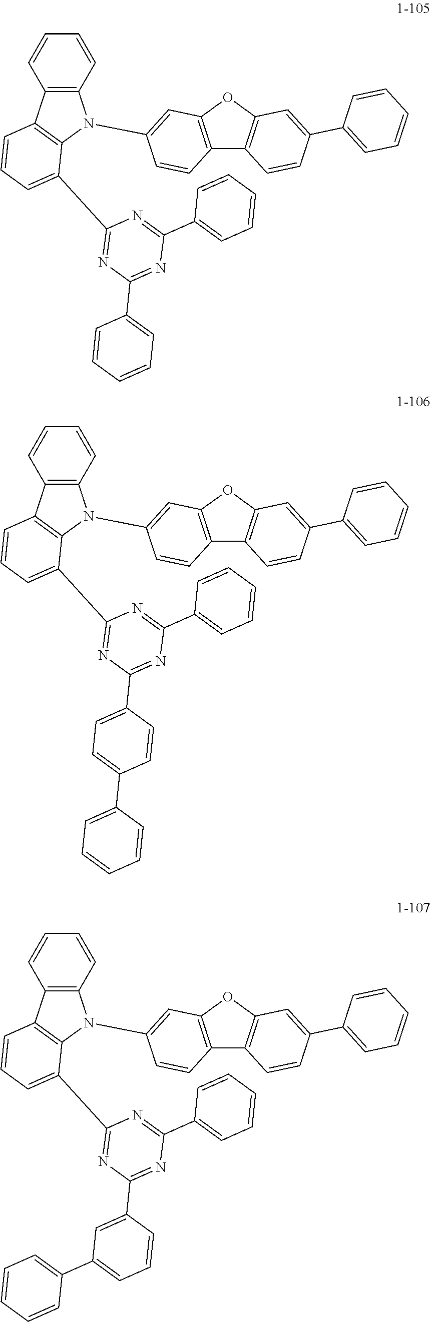 [SMILES: c1ccc(-c2ccc3c(c2)oc2cc(-n4c5ccccc5c5cccc(-c6nc(-c7ccccc7)nc(-c7ccccc7)n6)c54)ccc23)cc1 ; c1ccc(-c2ccc(-c3nc(-c4ccccc4)nc(-c4cccc5c6ccccc6n(-c6ccc7c(c6)oc6cc(-c8ccccc8)ccc67)c45)n3)cc2)cc1 ; c1ccc(-c2cccc(-c3nc(-c4ccccc4)nc(-c4cccc5c6ccccc6n(-c6ccc7c(c6)oc6cc(-c8ccccc8)ccc67)c45)n3)c2)cc1]

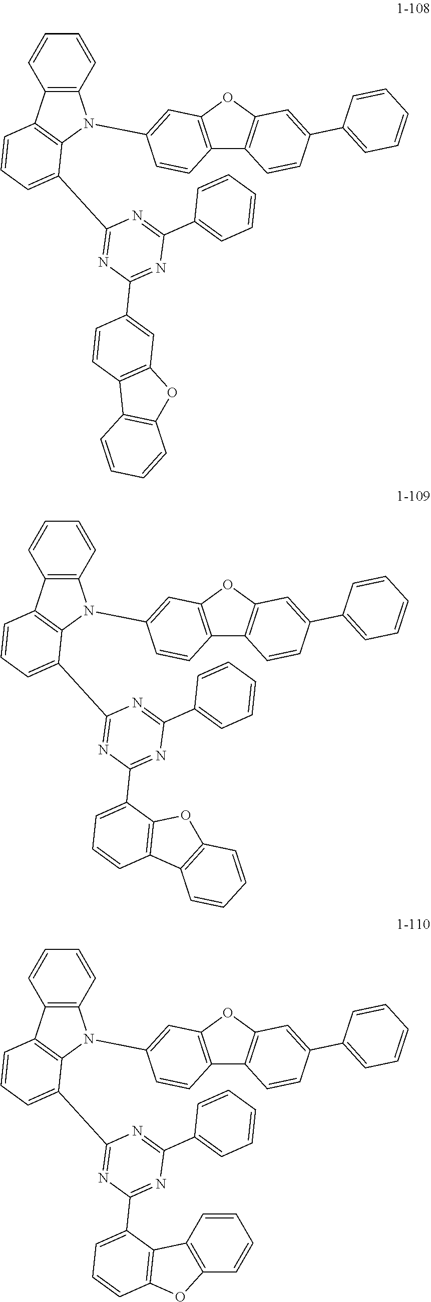 [SMILES: c1ccc(-c2ccc3c(c2)oc2cc(-n4c5ccccc5c5cccc(-c6nc(-c7ccccc7)nc(-c7ccc8c(c7)oc7ccccc78)n6)c54)ccc23)cc1 ; c1ccc(-c2ccc3c(c2)oc2cc(-n4c5ccccc5c5cccc(-c6nc(-c7ccccc7)nc(-c7cccc8c7oc7ccccc78)n6)c54)ccc23)cc1 ; c1ccc(-c2ccc3c(c2)oc2cc(-n4c5ccccc5c5cccc(-c6nc(-c7ccccc7)nc(-c7cccc8oc9ccccc9c78)n6)c54)ccc23)cc1]

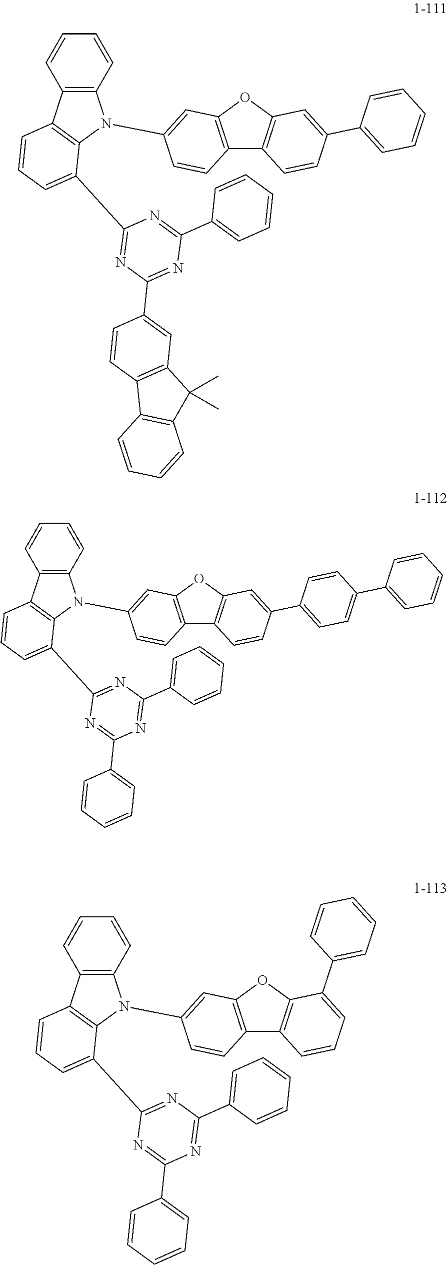 [SMILES: CC1(C)c2ccccc2-c2ccc(-c3nc(-c4ccccc4)nc(-c4cccc5c6ccccc6n(-c6ccc7c(c6)oc6cc(-c8ccccc8)ccc67)c45)n3)cc21 ; c1ccc(-c2ccc(-c3ccc4c(c3)oc3cc(-n5c6ccccc6c6cccc(-c7nc(-c8ccccc8)nc(-c8ccccc8)n7)c65)ccc34)cc2)cc1 ; c1ccc(-c2nc(-c3ccccc3)nc(-c3cccc4c5ccccc5n(-c5ccc6c(c5)oc5c(-c7ccccc7)cccc56)c34)n2)cc1]

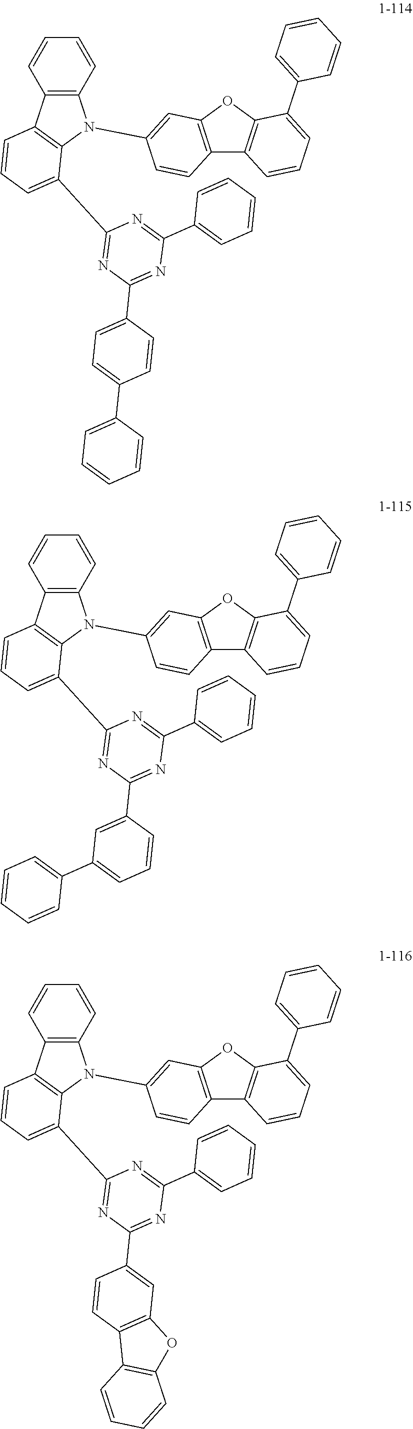 [SMILES: c1ccc(-c2ccc(-c3nc(-c4ccccc4)nc(-c4cccc5c6ccccc6n(-c6ccc7c(c6)oc6c(-c8ccccc8)cccc67)c45)n3)cc2)cc1 ; c1ccc(-c2cccc(-c3nc(-c4ccccc4)nc(-c4cccc5c6ccccc6n(-c6ccc7c(c6)oc6c(-c8ccccc8)cccc67)c45)n3)c2)cc1 ; c1ccc(-c2nc(-c3ccc4c(c3)oc3ccccc34)nc(-c3cccc4c5ccccc5n(-c5ccc6c(c5)oc5c(-c7ccccc7)cccc56)c34)n2)cc1]

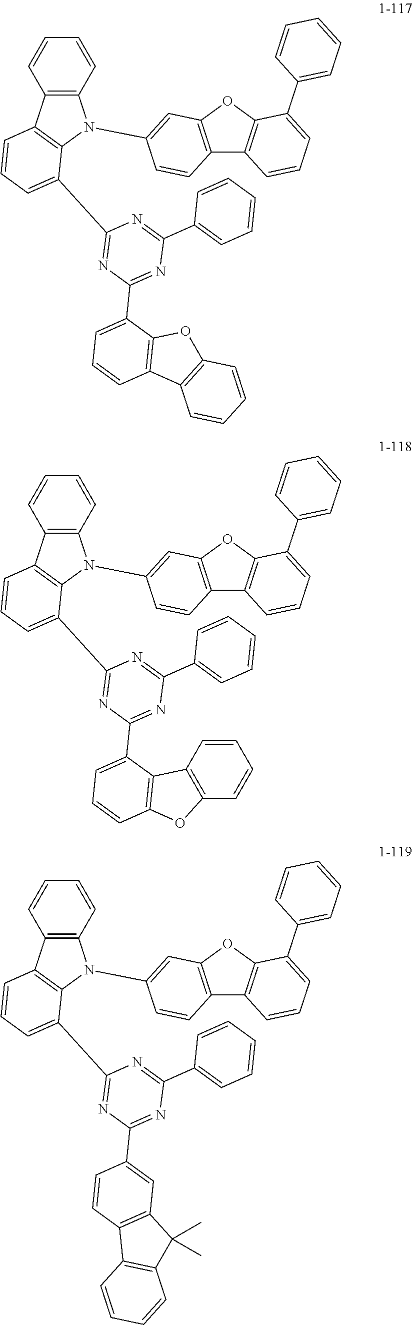 [SMILES: c1ccc(-c2nc(-c3cccc4c3oc3ccccc34)nc(-c3cccc4c5ccccc5n(-c5ccc6c(c5)oc5c(-c7ccccc7)cccc56)c34)n2)cc1 ; c1ccc(-c2nc(-c3cccc4oc5ccccc5c34)nc(-c3cccc4c5ccccc5n(-c5ccc6c(c5)oc5c(-c7ccccc7)cccc56)c34)n2)cc1 ; CC1(C)c2ccccc2-c2ccc(-c3nc(-c4ccccc4)nc(-c4cccc5c6ccccc6n(-c6ccc7c(c6)oc6c(-c8ccccc8)cccc67)c45)n3)cc21]

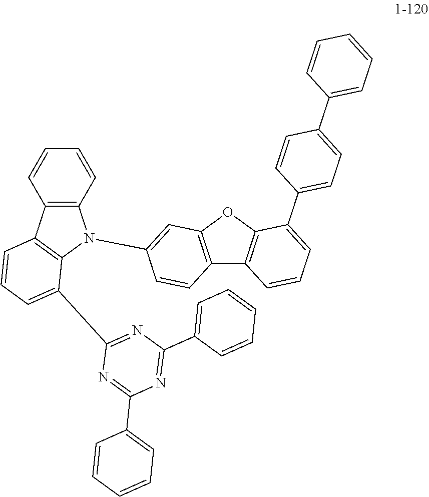 [SMILES: c1ccc(-c2ccc(-c3cccc4c3oc3cc(-n5c6ccccc6c6cccc(-c7nc(-c8ccccc8)nc(-c8ccccc8)n7)c65)ccc34)cc2)cc1]

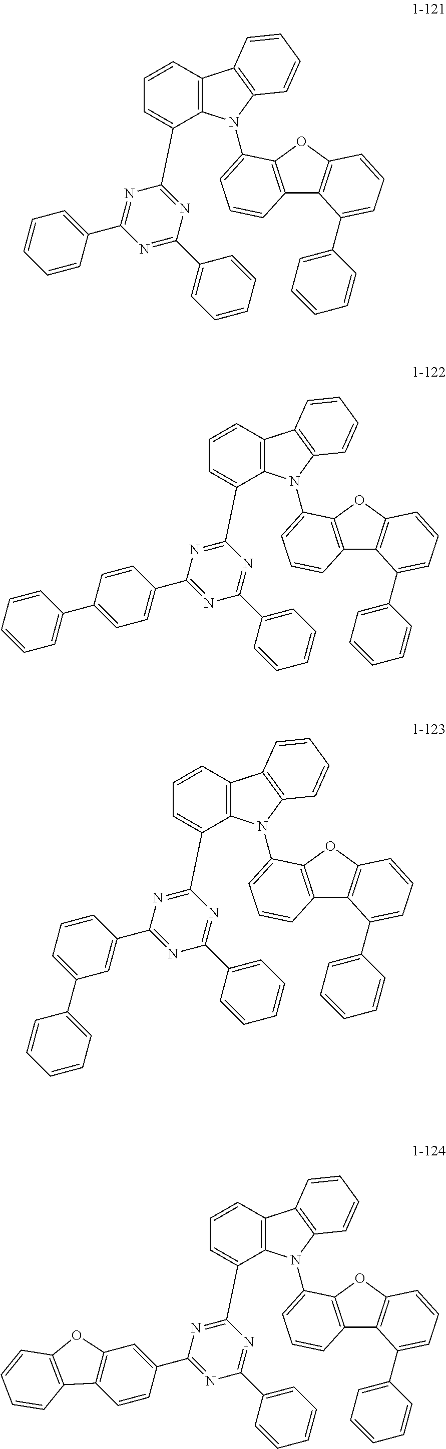 [SMILES: c1ccc(-c2nc(-c3ccccc3)nc(-c3cccc4c5ccccc5n(-c5cccc6c5oc5cccc(-c7ccccc7)c56)c34)n2)cc1 ; c1ccc(-c2ccc(-c3nc(-c4ccccc4)nc(-c4cccc5c6ccccc6n(-c6cccc7c6oc6cccc(-c8ccccc8)c67)c45)n3)cc2)cc1 ; c1ccc(-c2cccc(-c3nc(-c4ccccc4)nc(-c4cccc5c6ccccc6n(-c6cccc7c6oc6cccc(-c8ccccc8)c67)c45)n3)c2)cc1 ; c1ccc(-c2nc(-c3ccc4c(c3)oc3ccccc34)nc(-c3cccc4c5ccccc5n(-c5cccc6c5oc5cccc(-c7ccccc7)c56)c34)n2)cc1]

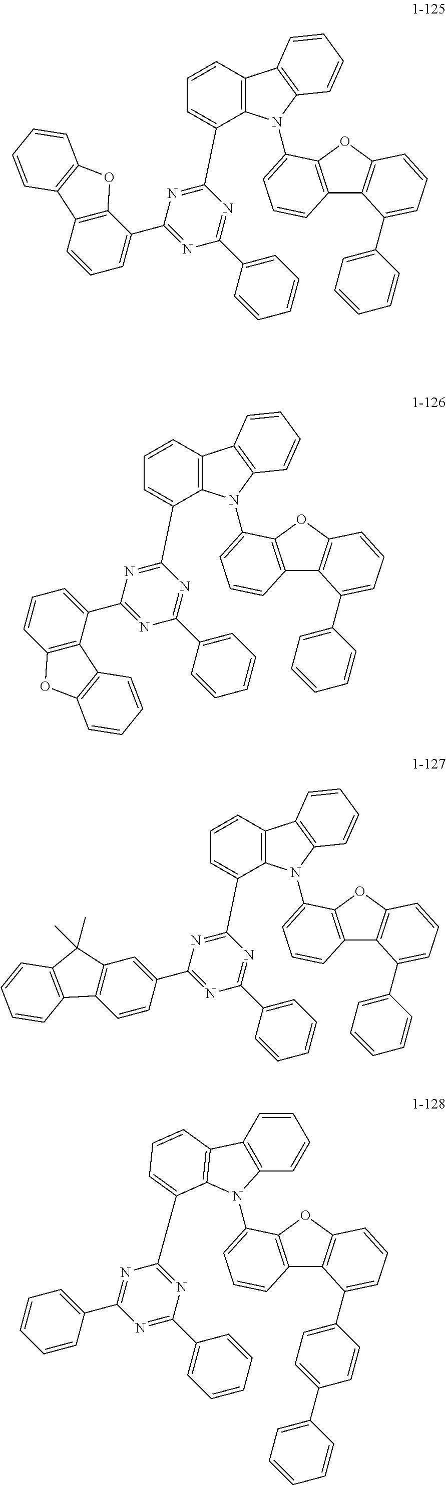 [SMILES: c1ccc(-c2nc(-c3cccc4c3oc3ccccc34)nc(-c3cccc4c5ccccc5n(-c5cccc6c5oc5cccc(-c7ccccc7)c56)c34)n2)cc1 ; c1ccc(-c2nc(-c3cccc4oc5ccccc5c34)nc(-c3cccc4c5ccccc5n(-c5cccc6c5oc5cccc(-c7ccccc7)c56)c34)n2)cc1 ; CC1(C)c2ccccc2-c2ccc(-c3nc(-c4ccccc4)nc(-c4cccc5c6ccccc6n(-c6cccc7c6oc6cccc(-c8ccccc8)c67)c45)n3)cc21 ; c1ccc(-c2ccc(-c3cccc4oc5c(-n6c7ccccc7c7cccc(-c8nc(-c9ccccc9)nc(-c9ccccc9)n8)c76)cccc5c34)cc2)cc1]

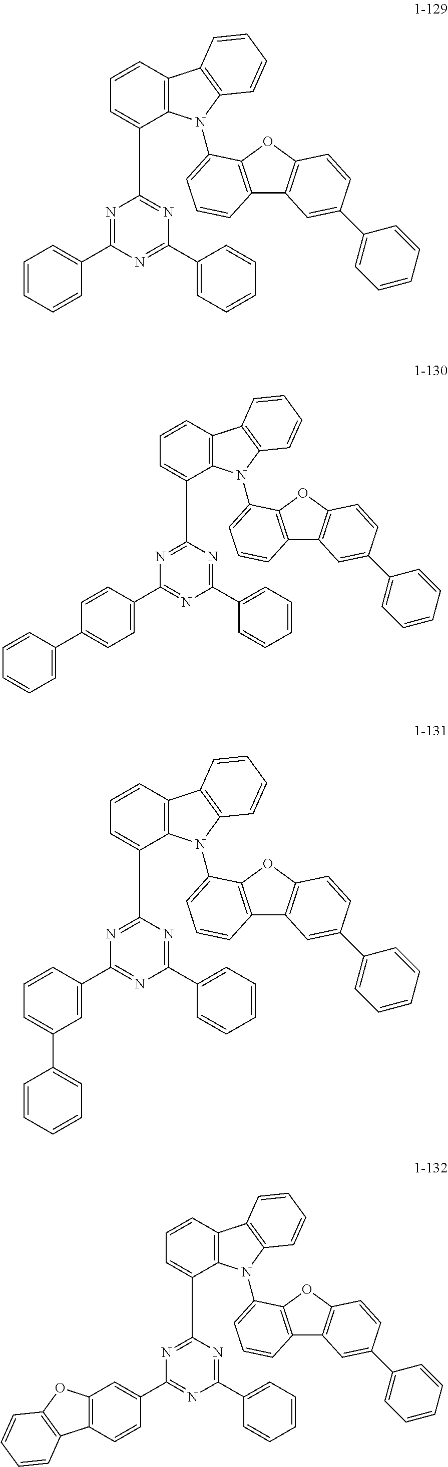 [SMILES: c1ccc(-c2ccc3oc4c(-n5c6ccccc6c6cccc(-c7nc(-c8ccccc8)nc(-c8ccccc8)n7)c65)cccc4c3c2)cc1 ; c1ccc(-c2ccc(-c3nc(-c4ccccc4)nc(-c4cccc5c6ccccc6n(-c6cccc7c6oc6ccc(-c8ccccc8)cc67)c45)n3)cc2)cc1 ; c1ccc(-c2cccc(-c3nc(-c4ccccc4)nc(-c4cccc5c6ccccc6n(-c6cccc7c6oc6ccc(-c8ccccc8)cc67)c45)n3)c2)cc1 ; c1ccc(-c2ccc3oc4c(-n5c6ccccc6c6cccc(-c7nc(-c8ccccc8)nc(-c8ccc9c(c8)oc8ccccc89)n7)c65)cccc4c3c2)cc1]

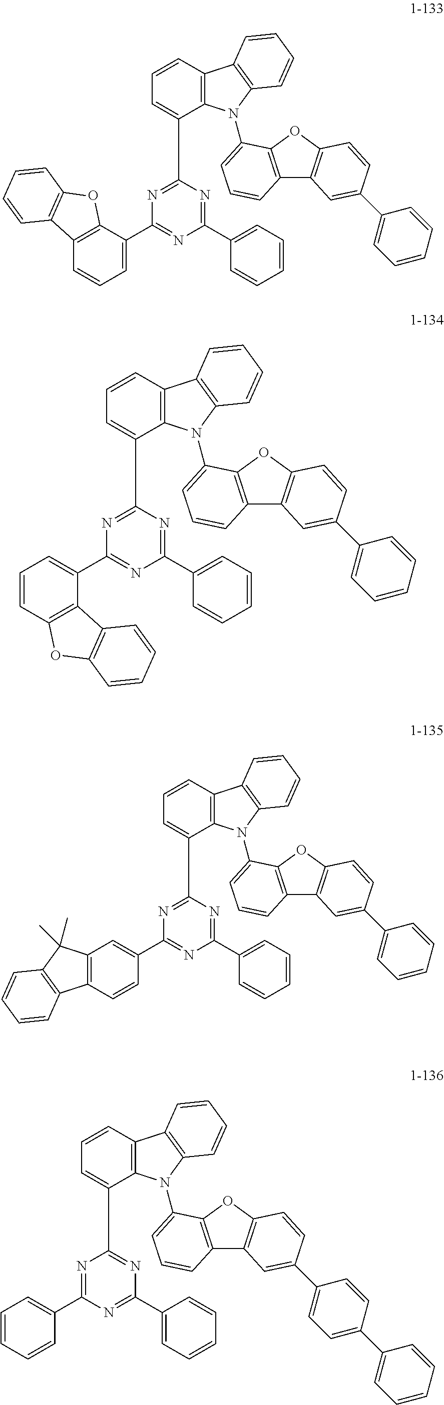 [SMILES: c1ccc(-c2ccc3oc4c(-n5c6ccccc6c6cccc(-c7nc(-c8ccccc8)nc(-c8cccc9c8oc8ccccc89)n7)c65)cccc4c3c2)cc1 ; c1ccc(-c2ccc3oc4c(-n5c6ccccc6c6cccc(-c7nc(-c8ccccc8)nc(-c8cccc9oc%10ccccc%10c89)n7)c65)cccc4c3c2)cc1 ; CC1(C)c2ccccc2-c2ccc(-c3nc(-c4ccccc4)nc(-c4cccc5c6ccccc6n(-c6cccc7c6oc6ccc(-c8ccccc8)cc67)c45)n3)cc21 ; c1ccc(-c2ccc(-c3ccc4oc5c(-n6c7ccccc7c7cccc(-c8nc(-c9ccccc9)nc(-c9ccccc9)n8)c76)cccc5c4c3)cc2)cc1]

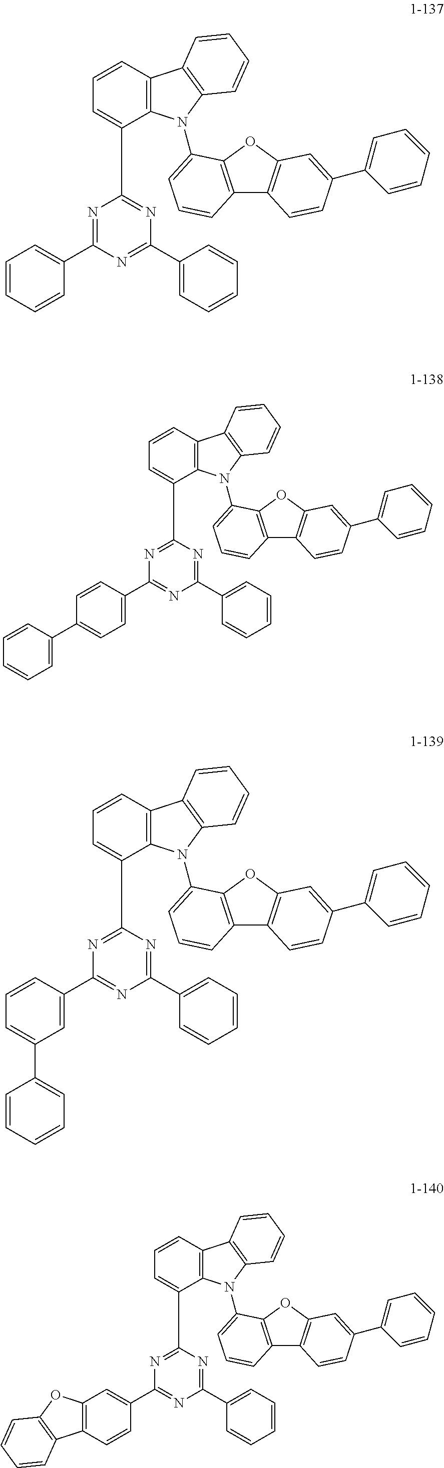 [SMILES: c1ccc(-c2ccc3c(c2)oc2c(-n4c5ccccc5c5cccc(-c6nc(-c7ccccc7)nc(-c7ccccc7)n6)c54)cccc23)cc1 ; c1ccc(-c2ccc(-c3nc(-c4ccccc4)nc(-c4cccc5c6ccccc6n(-c6cccc7c6oc6cc(-c8ccccc8)ccc67)c45)n3)cc2)cc1 ; c1ccc(-c2cccc(-c3nc(-c4ccccc4)nc(-c4cccc5c6ccccc6n(-c6cccc7c6oc6cc(-c8ccccc8)ccc67)c45)n3)c2)cc1 ; c1ccc(-c2ccc3c(c2)oc2c(-n4c5ccccc5c5cccc(-c6nc(-c7ccccc7)nc(-c7ccc8c(c7)oc7ccccc78)n6)c54)cccc23)cc1]

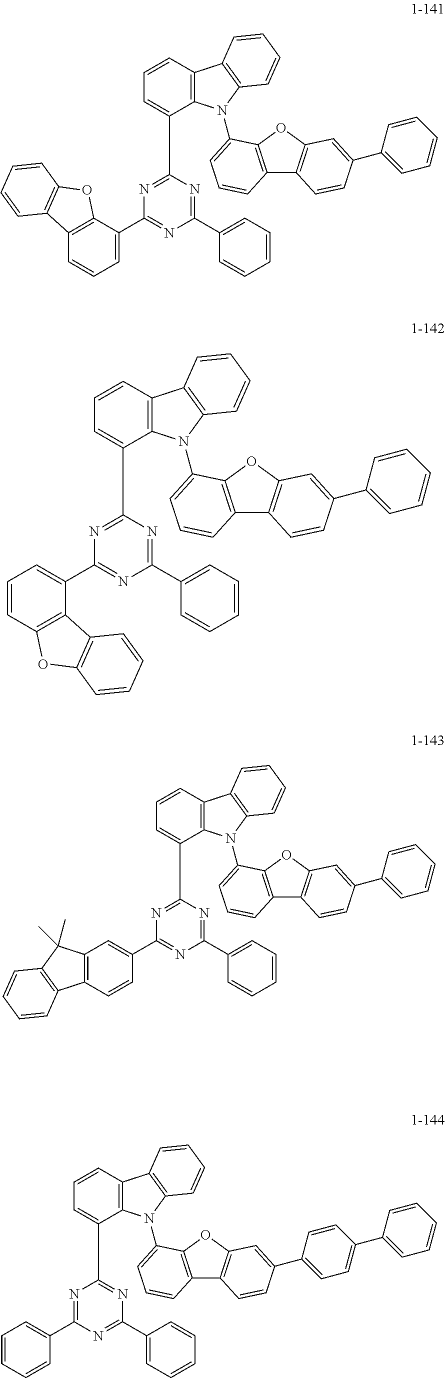 [SMILES: c1ccc(-c2ccc3c(c2)oc2c(-n4c5ccccc5c5cccc(-c6nc(-c7ccccc7)nc(-c7cccc8c7oc7ccccc78)n6)c54)cccc23)cc1 ; c1ccc(-c2ccc3c(c2)oc2c(-n4c5ccccc5c5cccc(-c6nc(-c7ccccc7)nc(-c7cccc8oc9ccccc9c78)n6)c54)cccc23)cc1 ; CC1(C)c2ccccc2-c2ccc(-c3nc(-c4ccccc4)nc(-c4cccc5c6ccccc6n(-c6cccc7c6oc6cc(-c8ccccc8)ccc67)c45)n3)cc21 ; c1ccc(-c2ccc(-c3ccc4c(c3)oc3c(-n5c6ccccc6c6cccc(-c7nc(-c8ccccc8)nc(-c8ccccc8)n7)c65)cccc34)cc2)cc1]

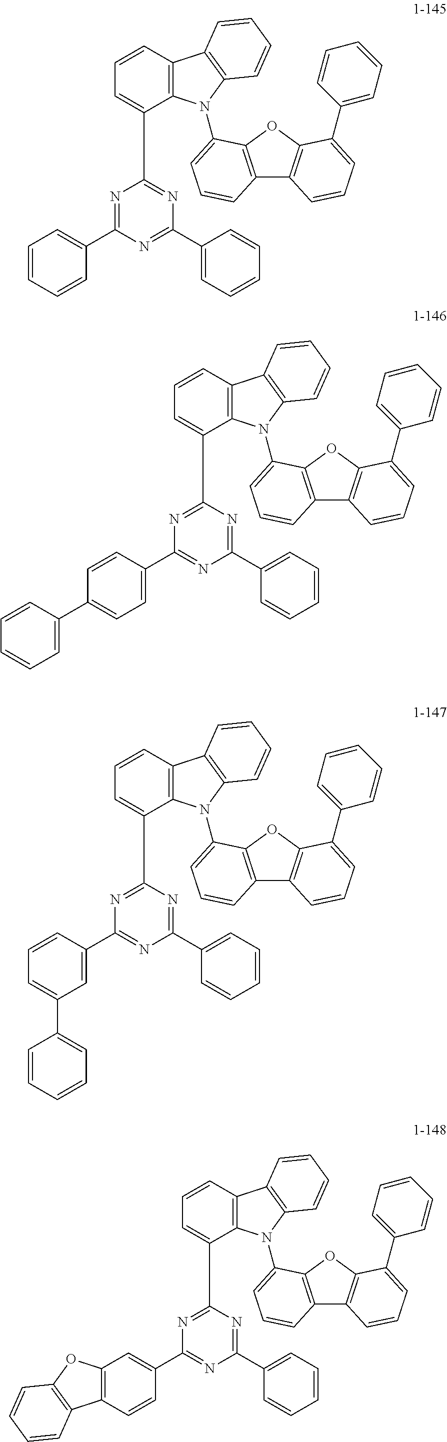 [SMILES: c1ccc(-c2nc(-c3ccccc3)nc(-c3cccc4c5ccccc5n(-c5cccc6c5oc5c(-c7ccccc7)cccc56)c34)n2)cc1 ; c1ccc(-c2ccc(-c3nc(-c4ccccc4)nc(-c4cccc5c6ccccc6n(-c6cccc7c6oc6c(-c8ccccc8)cccc67)c45)n3)cc2)cc1 ; c1ccc(-c2cccc(-c3nc(-c4ccccc4)nc(-c4cccc5c6ccccc6n(-c6cccc7c6oc6c(-c8ccccc8)cccc67)c45)n3)c2)cc1 ; c1ccc(-c2nc(-c3ccc4c(c3)oc3ccccc34)nc(-c3cccc4c5ccccc5n(-c5cccc6c5oc5c(-c7ccccc7)cccc56)c34)n2)cc1]

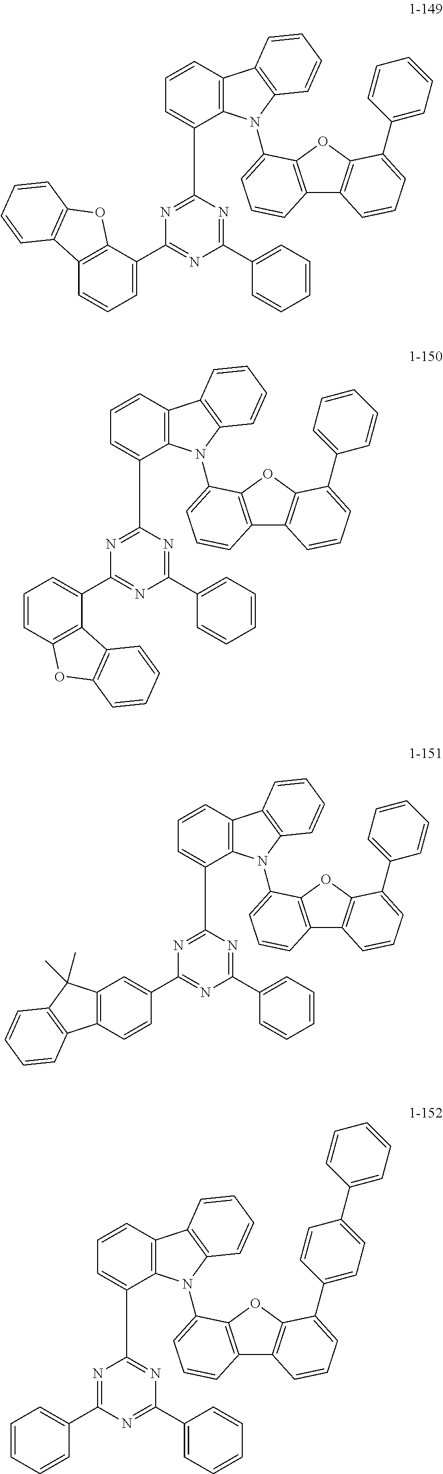 [SMILES: c1ccc(-c2nc(-c3cccc4c3oc3ccccc34)nc(-c3cccc4c5ccccc5n(-c5cccc6c5oc5c(-c7ccccc7)cccc56)c34)n2)cc1 ; c1ccc(-c2nc(-c3cccc4oc5ccccc5c34)nc(-c3cccc4c5ccccc5n(-c5cccc6c5oc5c(-c7ccccc7)cccc56)c34)n2)cc1 ; CC1(C)c2ccccc2-c2ccc(-c3nc(-c4ccccc4)nc(-c4cccc5c6ccccc6n(-c6cccc7c6oc6c(-c8ccccc8)cccc67)c45)n3)cc21 ; c1ccc(-c2ccc(-c3cccc4c3oc3c(-n5c6ccccc6c6cccc(-c7nc(-c8ccccc8)nc(-c8ccccc8)n7)c65)cccc34)cc2)cc1]

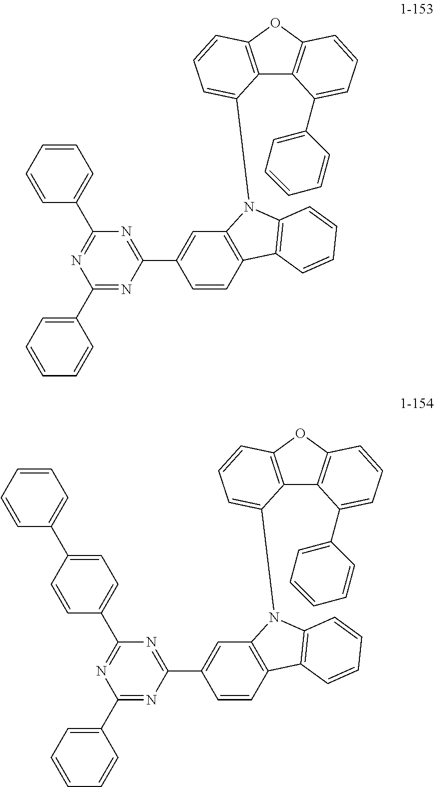 [SMILES: c1ccc(-c2nc(-c3ccccc3)nc(-c3ccc4c5ccccc5n(-c5cccc6oc7cccc(-c8ccccc8)c7c56)c4c3)n2)cc1 ; c1ccc(-c2ccc(-c3nc(-c4ccccc4)nc(-c4ccc5c6ccccc6n(-c6cccc7oc8cccc(-c9ccccc9)c8c67)c5c4)n3)cc2)cc1]

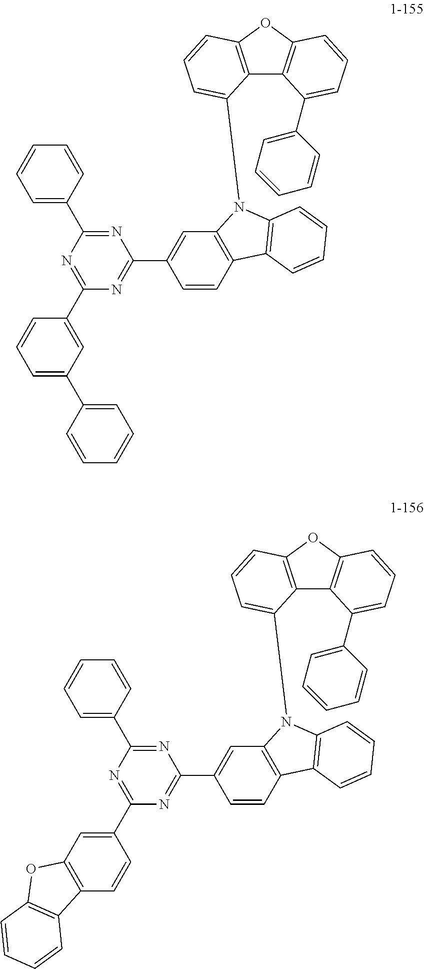 [SMILES: c1ccc(-c2cccc(-c3nc(-c4ccccc4)nc(-c4ccc5c6ccccc6n(-c6cccc7oc8cccc(-c9ccccc9)c8c67)c5c4)n3)c2)cc1 ; c1ccc(-c2nc(-c3ccc4c(c3)oc3ccccc34)nc(-c3ccc4c5ccccc5n(-c5cccc6oc7cccc(-c8ccccc8)c7c56)c4c3)n2)cc1]

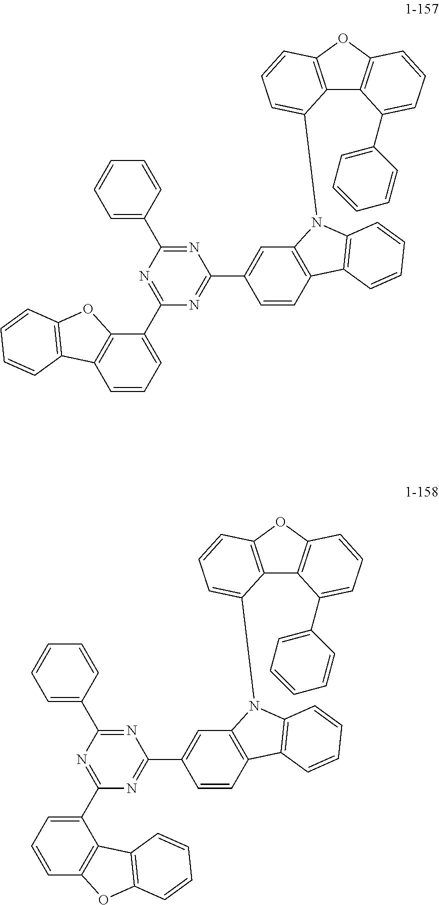 [SMILES: c1ccc(-c2nc(-c3ccc4c5ccccc5n(-c5cccc6oc7cccc(-c8ccccc8)c7c56)c4c3)nc(-c3cccc4c3oc3ccccc34)n2)cc1 ; c1ccc(-c2nc(-c3ccc4c5ccccc5n(-c5cccc6oc7cccc(-c8ccccc8)c7c56)c4c3)nc(-c3cccc4oc5ccccc5c34)n2)cc1]

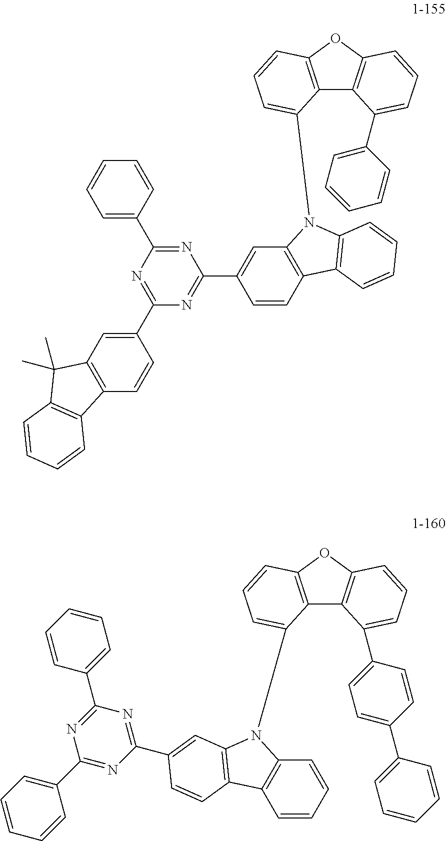 [SMILES: CC1(C)c2ccccc2-c2ccc(-c3nc(-c4ccccc4)nc(-c4ccc5c6ccccc6n(-c6cccc7oc8cccc(-c9ccccc9)c8c67)c5c4)n3)cc21 ; c1ccc(-c2ccc(-c3cccc4oc5cccc(-n6c7ccccc7c7ccc(-c8nc(-c9ccccc9)nc(-c9ccccc9)n8)cc76)c5c34)cc2)cc1]

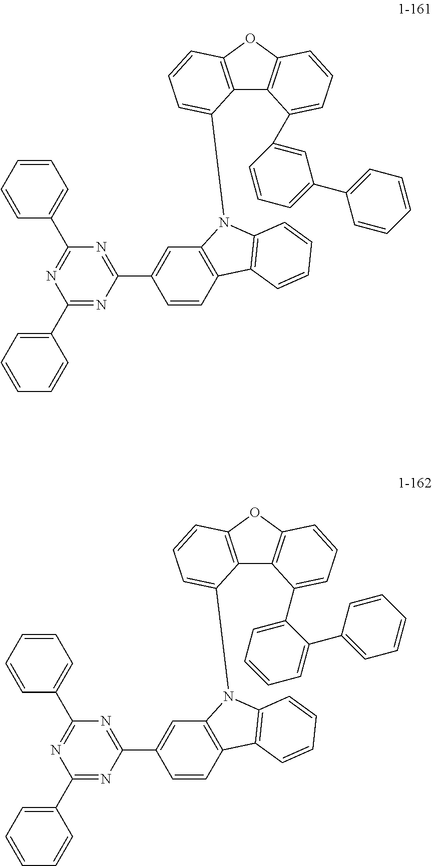 [SMILES: c1ccc(-c2cccc(-c3cccc4oc5cccc(-n6c7ccccc7c7ccc(-c8nc(-c9ccccc9)nc(-c9ccccc9)n8)cc76)c5c34)c2)cc1 ; c1ccc(-c2nc(-c3ccccc3)nc(-c3ccc4c5ccccc5n(-c5cccc6oc7cccc(-c8ccccc8-c8ccccc8)c7c56)c4c3)n2)cc1]

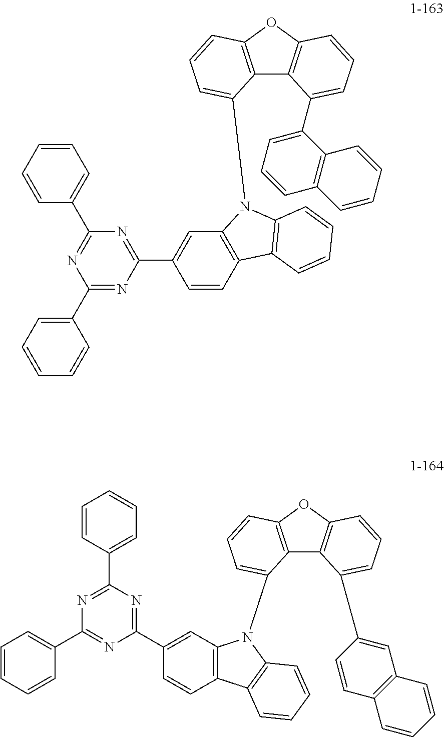 [SMILES: c1ccc(-c2nc(-c3ccccc3)nc(-c3ccc4c5ccccc5n(-c5cccc6oc7cccc(-c8cccc9ccccc89)c7c56)c4c3)n2)cc1 ; c1ccc(-c2nc(-c3ccccc3)nc(-c3ccc4c5ccccc5n(-c5cccc6oc7cccc(-c8ccc9ccccc9c8)c7c56)c4c3)n2)cc1]

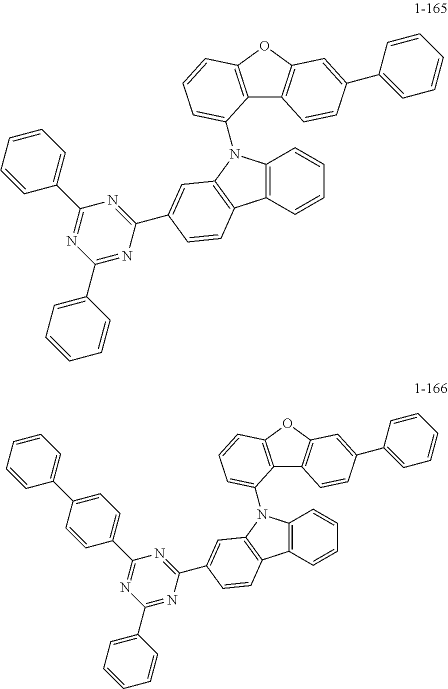 [SMILES: c1ccc(-c2ccc3c(c2)oc2cccc(-n4c5ccccc5c5ccc(-c6nc(-c7ccccc7)nc(-c7ccccc7)n6)cc54)c23)cc1 ; c1ccc(-c2ccc(-c3nc(-c4ccccc4)nc(-c4ccc5c6ccccc6n(-c6cccc7oc8cc(-c9ccccc9)ccc8c67)c5c4)n3)cc2)cc1]

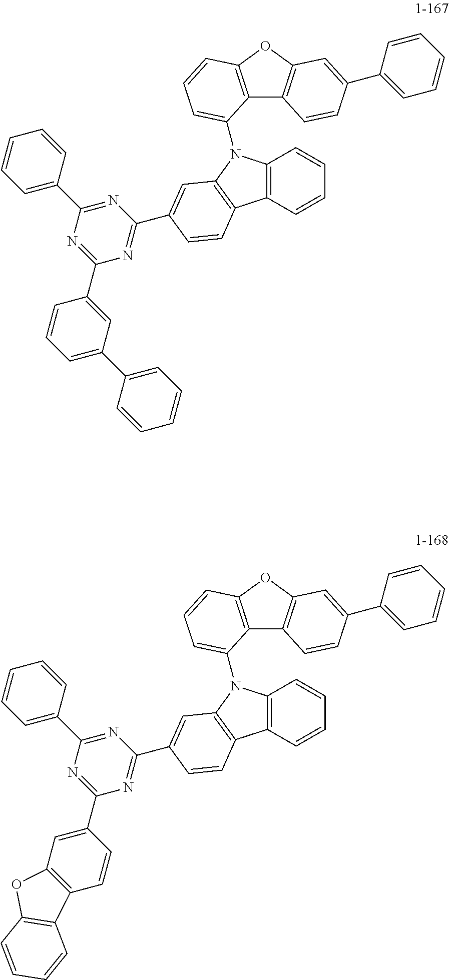 [SMILES: c1ccc(-c2cccc(-c3nc(-c4ccccc4)nc(-c4ccc5c6ccccc6n(-c6cccc7oc8cc(-c9ccccc9)ccc8c67)c5c4)n3)c2)cc1 ; c1ccc(-c2ccc3c(c2)oc2cccc(-n4c5ccccc5c5ccc(-c6nc(-c7ccccc7)nc(-c7ccc8c(c7)oc7ccccc78)n6)cc54)c23)cc1]

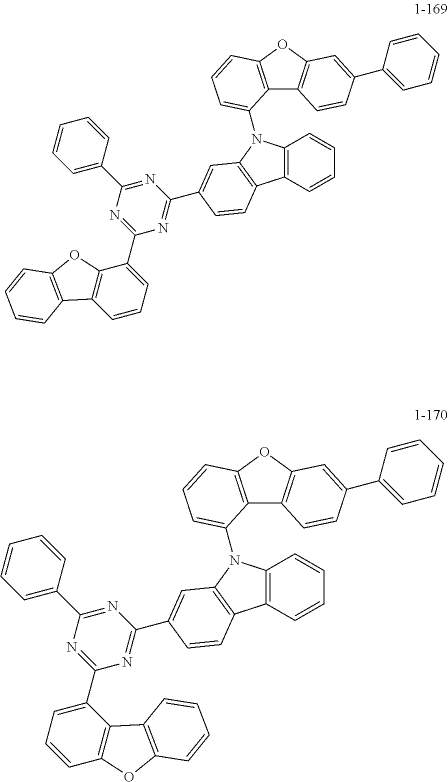 [SMILES: c1ccc(-c2ccc3c(c2)oc2cccc(-n4c5ccccc5c5ccc(-c6nc(-c7ccccc7)nc(-c7cccc8c7oc7ccccc78)n6)cc54)c23)cc1 ; c1ccc(-c2ccc3c(c2)oc2cccc(-n4c5ccccc5c5ccc(-c6nc(-c7ccccc7)nc(-c7cccc8oc9ccccc9c78)n6)cc54)c23)cc1]

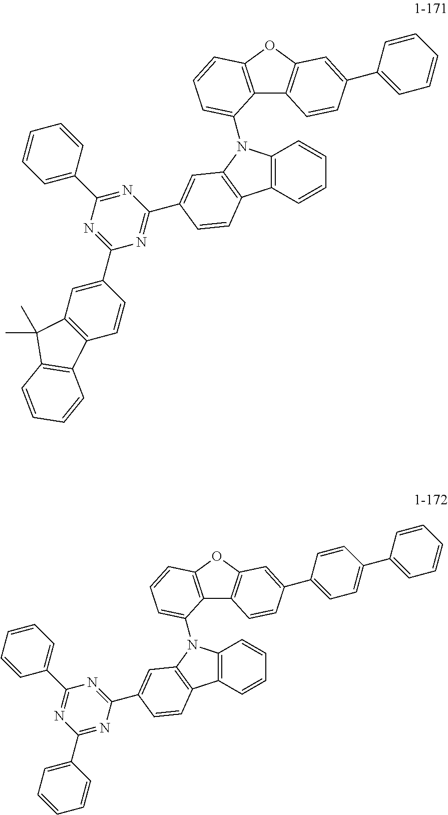 [SMILES: CC1(C)c2ccccc2-c2ccc(-c3nc(-c4ccccc4)nc(-c4ccc5c6ccccc6n(-c6cccc7oc8cc(-c9ccccc9)ccc8c67)c5c4)n3)cc21 ; c1ccc(-c2ccc(-c3ccc4c(c3)oc3cccc(-n5c6ccccc6c6ccc(-c7nc(-c8ccccc8)nc(-c8ccccc8)n7)cc65)c34)cc2)cc1]

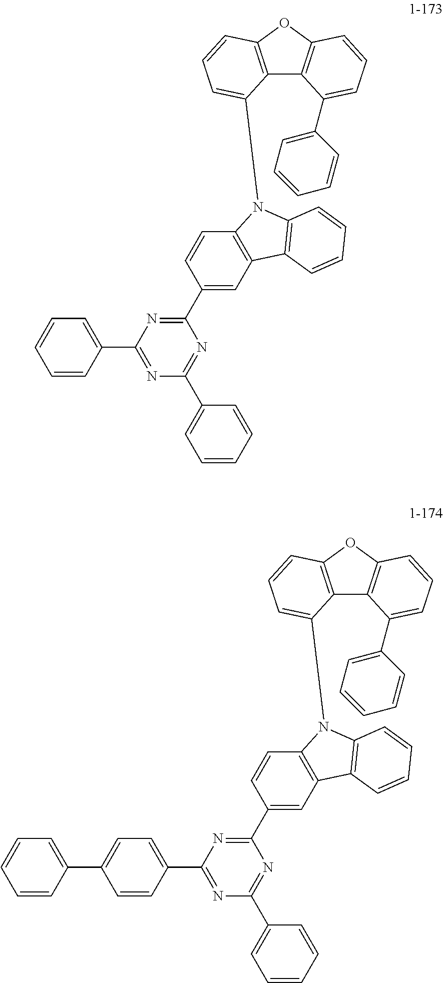 [SMILES: c1ccc(-c2nc(-c3ccccc3)nc(-c3ccc4c(c3)c3ccccc3n4-c3cccc4oc5cccc(-c6ccccc6)c5c34)n2)cc1 ; c1ccc(-c2ccc(-c3nc(-c4ccccc4)nc(-c4ccc5c(c4)c4ccccc4n5-c4cccc5oc6cccc(-c7ccccc7)c6c45)n3)cc2)cc1]

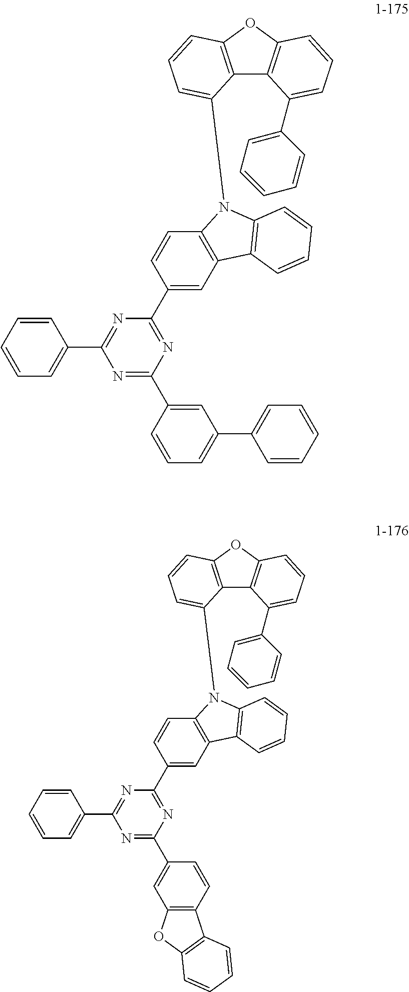 [SMILES: c1ccc(-c2cccc(-c3nc(-c4ccccc4)nc(-c4ccc5c(c4)c4ccccc4n5-c4cccc5oc6cccc(-c7ccccc7)c6c45)n3)c2)cc1 ; c1ccc(-c2nc(-c3ccc4c(c3)oc3ccccc34)nc(-c3ccc4c(c3)c3ccccc3n4-c3cccc4oc5cccc(-c6ccccc6)c5c34)n2)cc1]

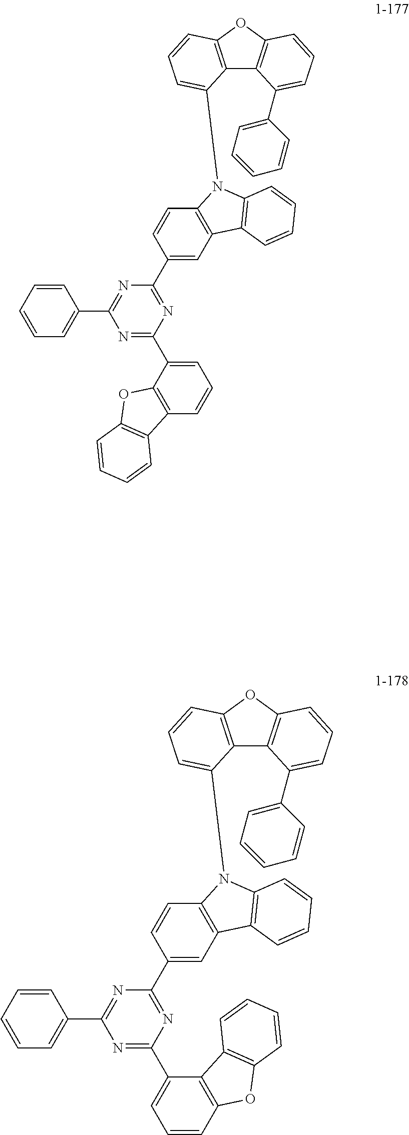 [SMILES: c1ccc(-c2nc(-c3ccc4c(c3)c3ccccc3n4-c3cccc4oc5cccc(-c6ccccc6)c5c34)nc(-c3cccc4c3oc3ccccc34)n2)cc1 ; c1ccc(-c2nc(-c3ccc4c(c3)c3ccccc3n4-c3cccc4oc5cccc(-c6ccccc6)c5c34)nc(-c3cccc4oc5ccccc5c34)n2)cc1]

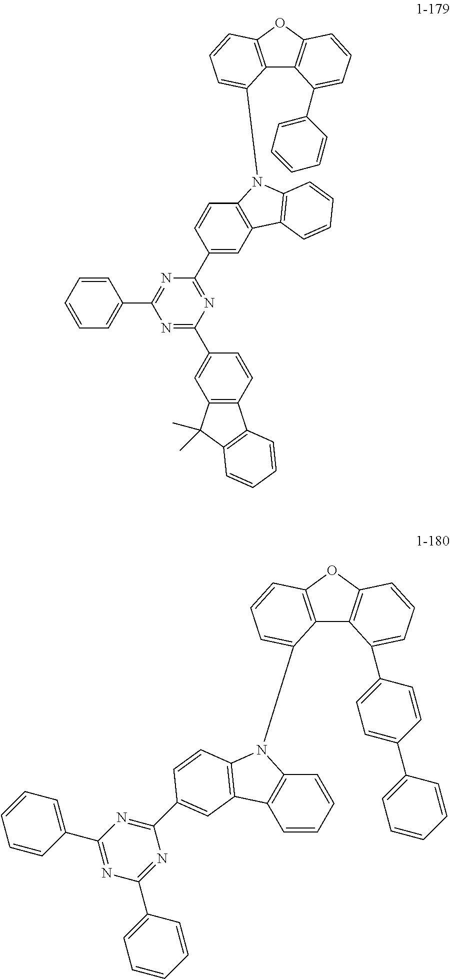 [SMILES: CC1(C)c2ccccc2-c2ccc(-c3nc(-c4ccccc4)nc(-c4ccc5c(c4)c4ccccc4n5-c4cccc5oc6cccc(-c7ccccc7)c6c45)n3)cc21 ; c1ccc(-c2ccc(-c3cccc4oc5cccc(-n6c7ccccc7c7cc(-c8nc(-c9ccccc9)nc(-c9ccccc9)n8)ccc76)c5c34)cc2)cc1]

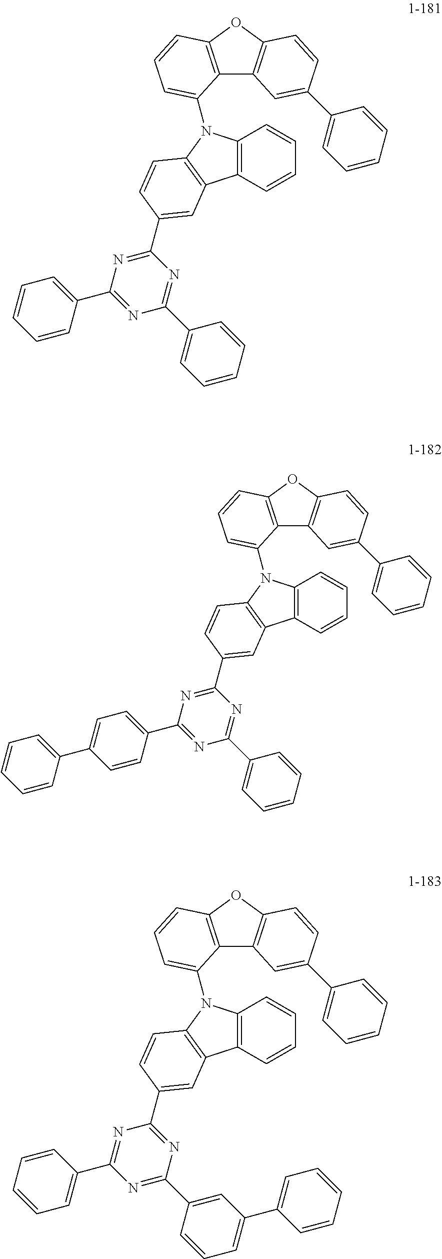 [SMILES: c1ccc(-c2ccc3oc4cccc(-n5c6ccccc6c6cc(-c7nc(-c8ccccc8)nc(-c8ccccc8)n7)ccc65)c4c3c2)cc1 ; c1ccc(-c2ccc(-c3nc(-c4ccccc4)nc(-c4ccc5c(c4)c4ccccc4n5-c4cccc5oc6ccc(-c7ccccc7)cc6c45)n3)cc2)cc1 ; c1ccc(-c2cccc(-c3nc(-c4ccccc4)nc(-c4ccc5c(c4)c4ccccc4n5-c4cccc5oc6ccc(-c7ccccc7)cc6c45)n3)c2)cc1]

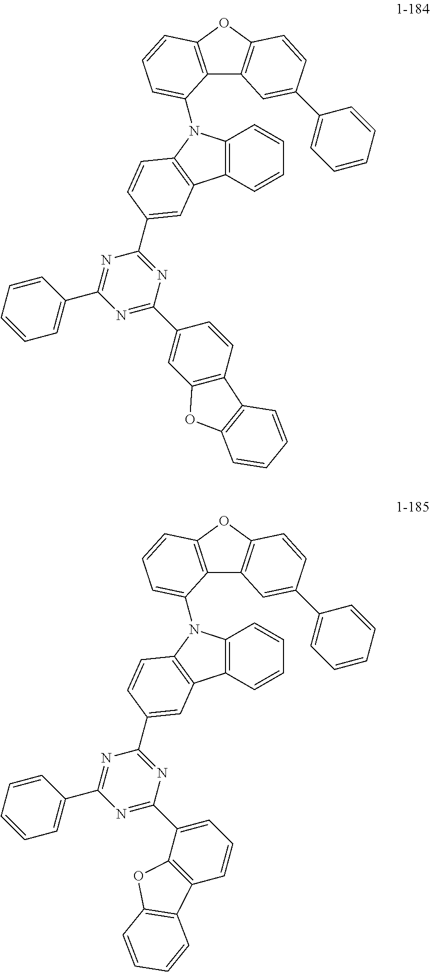 [SMILES: c1ccc(-c2ccc3oc4cccc(-n5c6ccccc6c6cc(-c7nc(-c8ccccc8)nc(-c8ccc9c(c8)oc8ccccc89)n7)ccc65)c4c3c2)cc1 ; c1ccc(-c2ccc3oc4cccc(-n5c6ccccc6c6cc(-c7nc(-c8ccccc8)nc(-c8cccc9c8oc8ccccc89)n7)ccc65)c4c3c2)cc1]

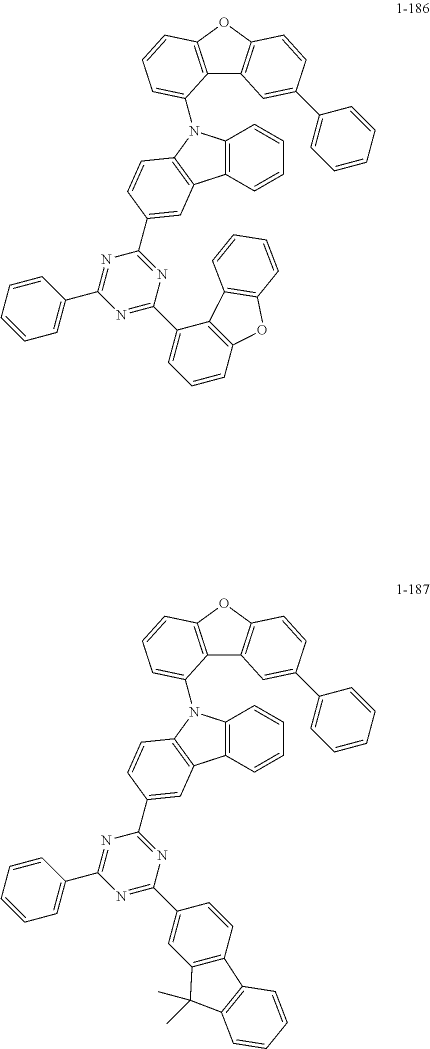 [SMILES: c1ccc(-c2ccc3oc4cccc(-n5c6ccccc6c6cc(-c7nc(-c8ccccc8)nc(-c8cccc9oc%10ccccc%10c89)n7)ccc65)c4c3c2)cc1 ; CC1(C)c2ccccc2-c2ccc(-c3nc(-c4ccccc4)nc(-c4ccc5c(c4)c4ccccc4n5-c4cccc5oc6ccc(-c7ccccc7)cc6c45)n3)cc21]

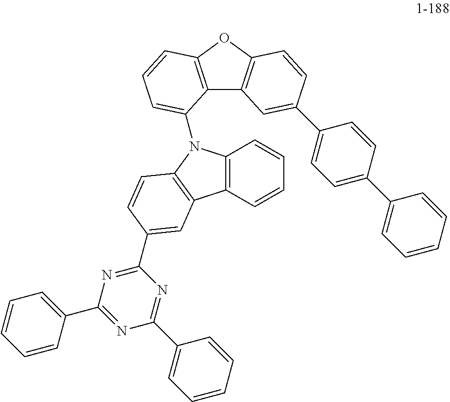 [SMILES: c1ccc(-c2ccc(-c3ccc4oc5cccc(-n6c7ccccc7c7cc(-c8nc(-c9ccccc9)nc(-c9ccccc9)n8)ccc76)c5c4c3)cc2)cc1]

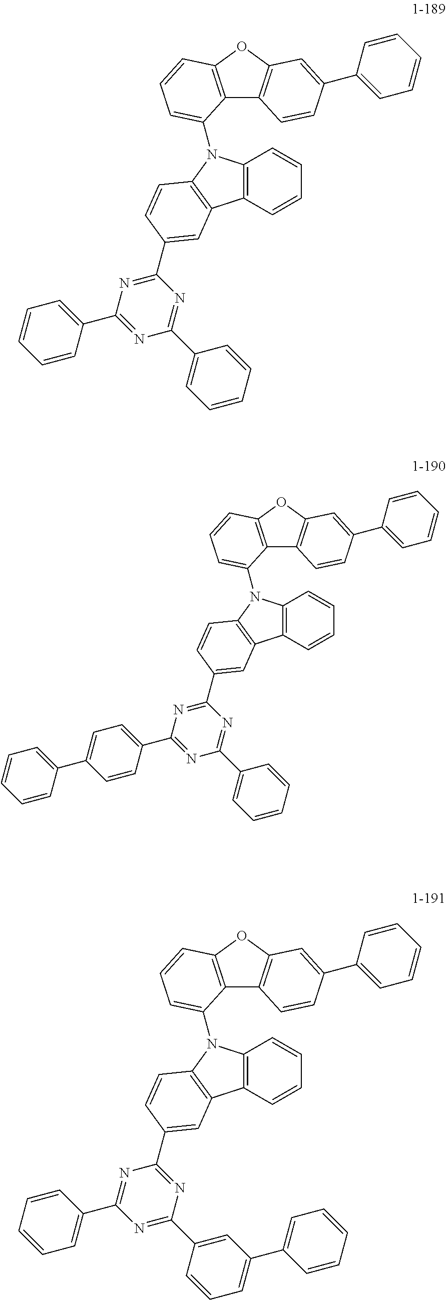 [SMILES: c1ccc(-c2ccc3c(c2)oc2cccc(-n4c5ccccc5c5cc(-c6nc(-c7ccccc7)nc(-c7ccccc7)n6)ccc54)c23)cc1 ; c1ccc(-c2ccc(-c3nc(-c4ccccc4)nc(-c4ccc5c(c4)c4ccccc4n5-c4cccc5oc6cc(-c7ccccc7)ccc6c45)n3)cc2)cc1 ; c1ccc(-c2cccc(-c3nc(-c4ccccc4)nc(-c4ccc5c(c4)c4ccccc4n5-c4cccc5oc6cc(-c7ccccc7)ccc6c45)n3)c2)cc1]

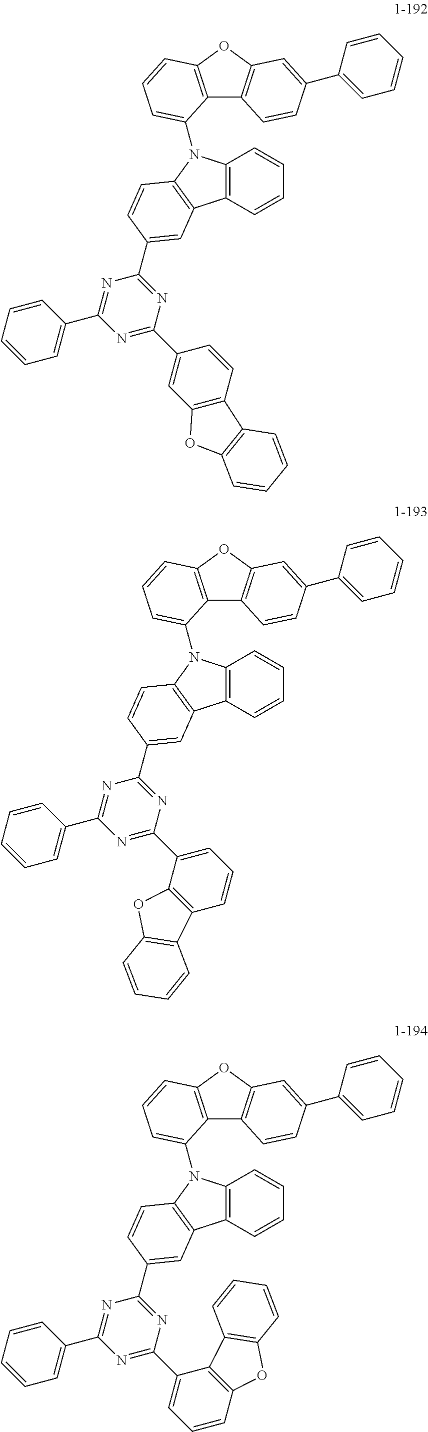 [SMILES: c1ccc(-c2ccc3c(c2)oc2cccc(-n4c5ccccc5c5cc(-c6nc(-c7ccccc7)nc(-c7ccc8c(c7)oc7ccccc78)n6)ccc54)c23)cc1 ; c1ccc(-c2ccc3c(c2)oc2cccc(-n4c5ccccc5c5cc(-c6nc(-c7ccccc7)nc(-c7cccc8c7oc7ccccc78)n6)ccc54)c23)cc1 ; c1ccc(-c2ccc3c(c2)oc2cccc(-n4c5ccccc5c5cc(-c6nc(-c7ccccc7)nc(-c7cccc8oc9ccccc9c78)n6)ccc54)c23)cc1]

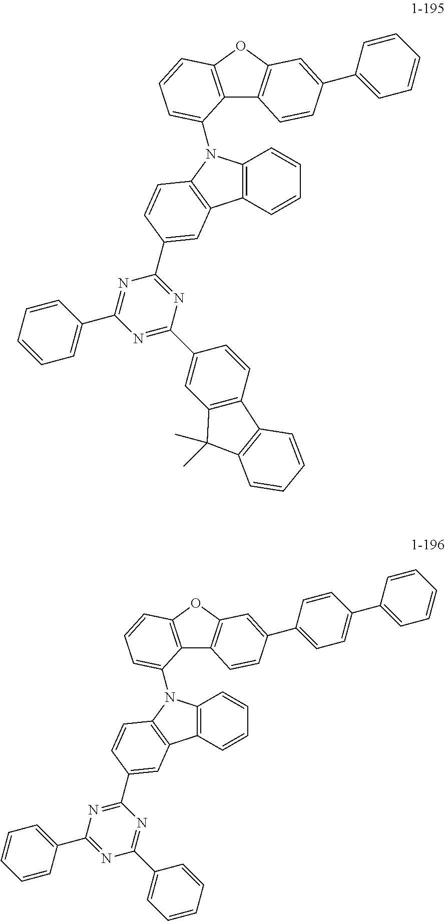 [SMILES: CC1(C)c2ccccc2-c2ccc(-c3nc(-c4ccccc4)nc(-c4ccc5c(c4)c4ccccc4n5-c4cccc5oc6cc(-c7ccccc7)ccc6c45)n3)cc21 ; c1ccc(-c2ccc(-c3ccc4c(c3)oc3cccc(-n5c6ccccc6c6cc(-c7nc(-c8ccccc8)nc(-c8ccccc8)n7)ccc65)c34)cc2)cc1]

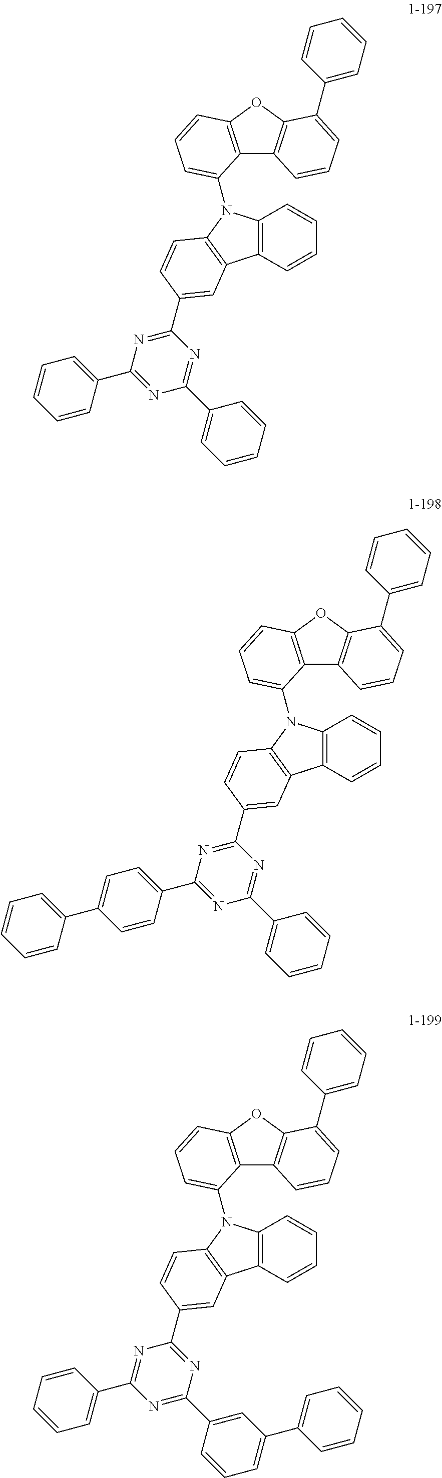 [SMILES: c1ccc(-c2nc(-c3ccccc3)nc(-c3ccc4c(c3)c3ccccc3n4-c3cccc4oc5c(-c6ccccc6)cccc5c34)n2)cc1 ; c1ccc(-c2ccc(-c3nc(-c4ccccc4)nc(-c4ccc5c(c4)c4ccccc4n5-c4cccc5oc6c(-c7ccccc7)cccc6c45)n3)cc2)cc1 ; c1ccc(-c2cccc(-c3nc(-c4ccccc4)nc(-c4ccc5c(c4)c4ccccc4n5-c4cccc5oc6c(-c7ccccc7)cccc6c45)n3)c2)cc1]

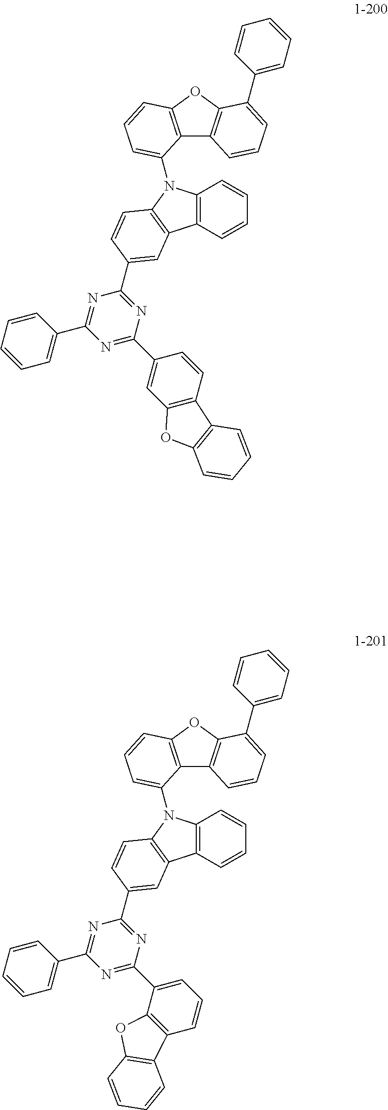 [SMILES: c1ccc(-c2nc(-c3ccc4c(c3)oc3ccccc34)nc(-c3ccc4c(c3)c3ccccc3n4-c3cccc4oc5c(-c6ccccc6)cccc5c34)n2)cc1 ; c1ccc(-c2nc(-c3ccc4c(c3)c3ccccc3n4-c3cccc4oc5c(-c6ccccc6)cccc5c34)nc(-c3cccc4c3oc3ccccc34)n2)cc1]

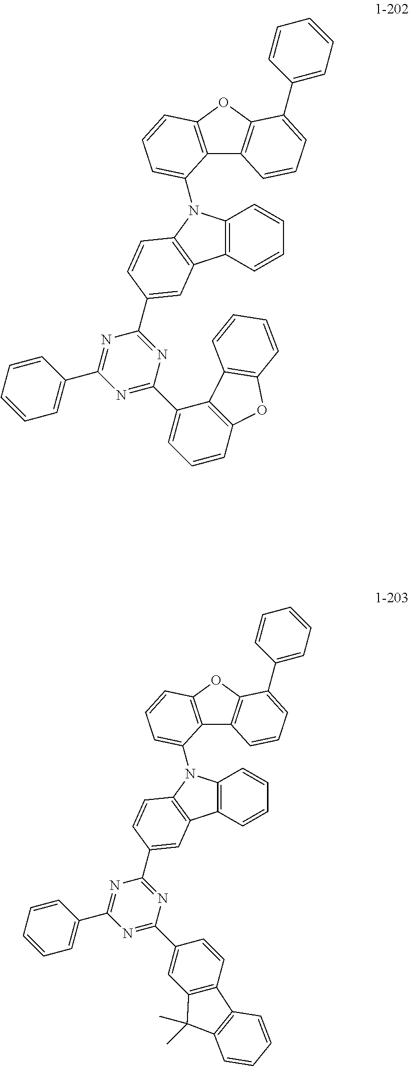 [SMILES: c1ccc(-c2nc(-c3ccc4c(c3)c3ccccc3n4-c3cccc4oc5c(-c6ccccc6)cccc5c34)nc(-c3cccc4oc5ccccc5c34)n2)cc1 ; CC1(C)c2ccccc2-c2ccc(-c3nc(-c4ccccc4)nc(-c4ccc5c(c4)c4ccccc4n5-c4cccc5oc6c(-c7ccccc7)cccc6c45)n3)cc21]

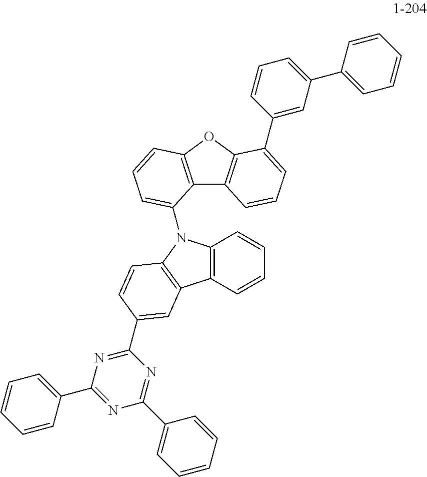 [SMILES: c1ccc(-c2cccc(-c3cccc4c3oc3cccc(-n5c6ccccc6c6cc(-c7nc(-c8ccccc8)nc(-c8ccccc8)n7)ccc65)c34)c2)cc1]

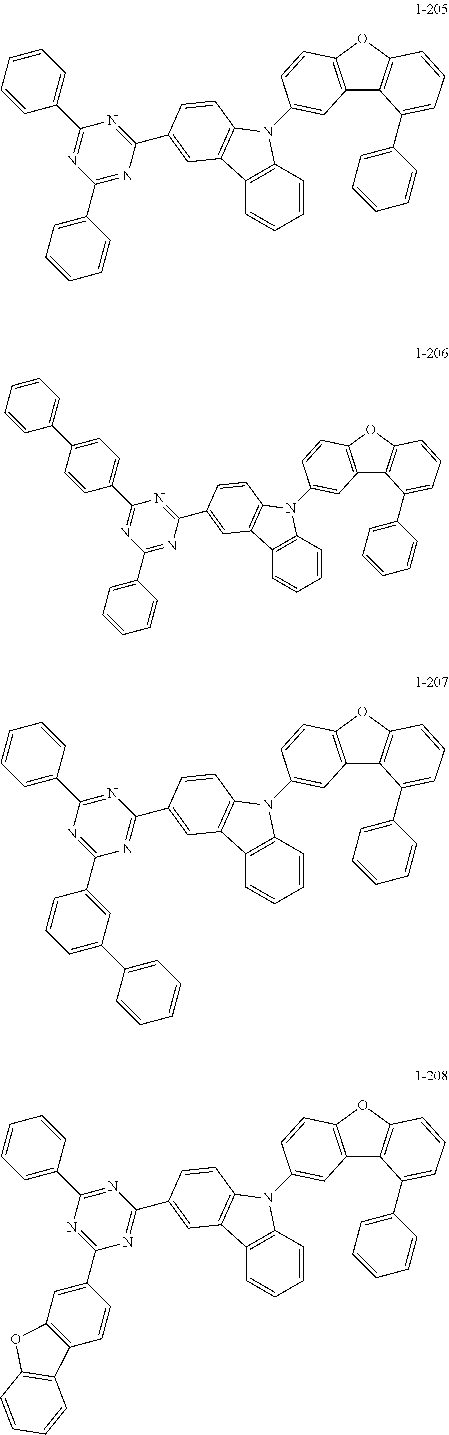 [SMILES: c1ccc(-c2nc(-c3ccccc3)nc(-c3ccc4c(c3)c3ccccc3n4-c3ccc4oc5cccc(-c6ccccc6)c5c4c3)n2)cc1 ; c1ccc(-c2ccc(-c3nc(-c4ccccc4)nc(-c4ccc5c(c4)c4ccccc4n5-c4ccc5oc6cccc(-c7ccccc7)c6c5c4)n3)cc2)cc1 ; c1ccc(-c2cccc(-c3nc(-c4ccccc4)nc(-c4ccc5c(c4)c4ccccc4n5-c4ccc5oc6cccc(-c7ccccc7)c6c5c4)n3)c2)cc1 ; c1ccc(-c2nc(-c3ccc4c(c3)oc3ccccc34)nc(-c3ccc4c(c3)c3ccccc3n4-c3ccc4oc5cccc(-c6ccccc6)c5c4c3)n2)cc1]

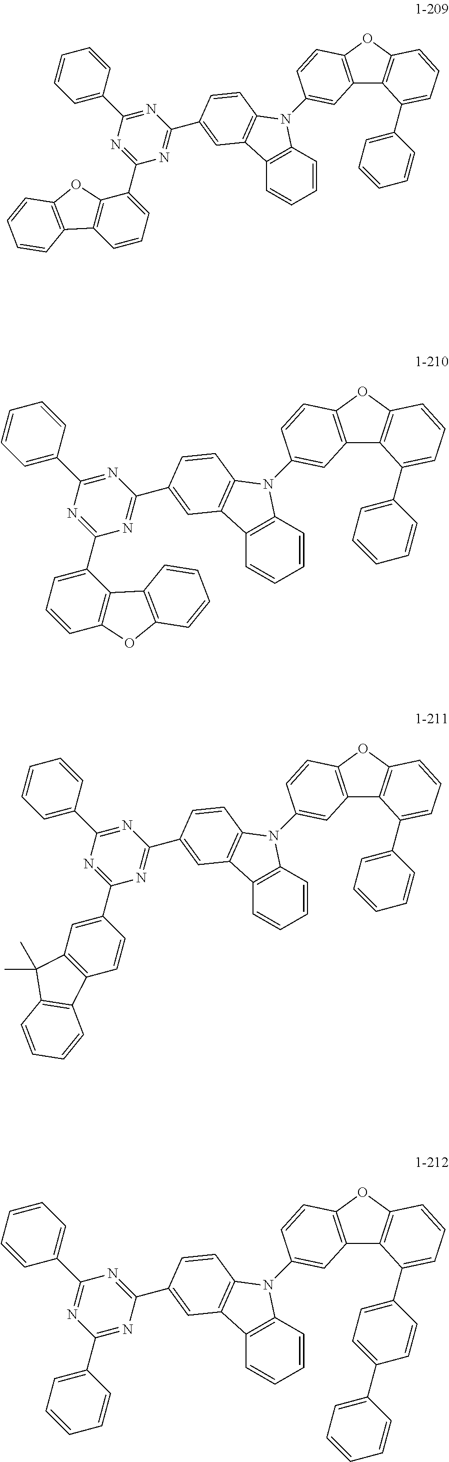 [SMILES: c1ccc(-c2nc(-c3ccc4c(c3)c3ccccc3n4-c3ccc4oc5cccc(-c6ccccc6)c5c4c3)nc(-c3cccc4c3oc3ccccc34)n2)cc1 ; c1ccc(-c2nc(-c3ccc4c(c3)c3ccccc3n4-c3ccc4oc5cccc(-c6ccccc6)c5c4c3)nc(-c3cccc4oc5ccccc5c34)n2)cc1 ; CC1(C)c2ccccc2-c2ccc(-c3nc(-c4ccccc4)nc(-c4ccc5c(c4)c4ccccc4n5-c4ccc5oc6cccc(-c7ccccc7)c6c5c4)n3)cc21 ; c1ccc(-c2ccc(-c3cccc4oc5ccc(-n6c7ccccc7c7cc(-c8nc(-c9ccccc9)nc(-c9ccccc9)n8)ccc76)cc5c34)cc2)cc1]

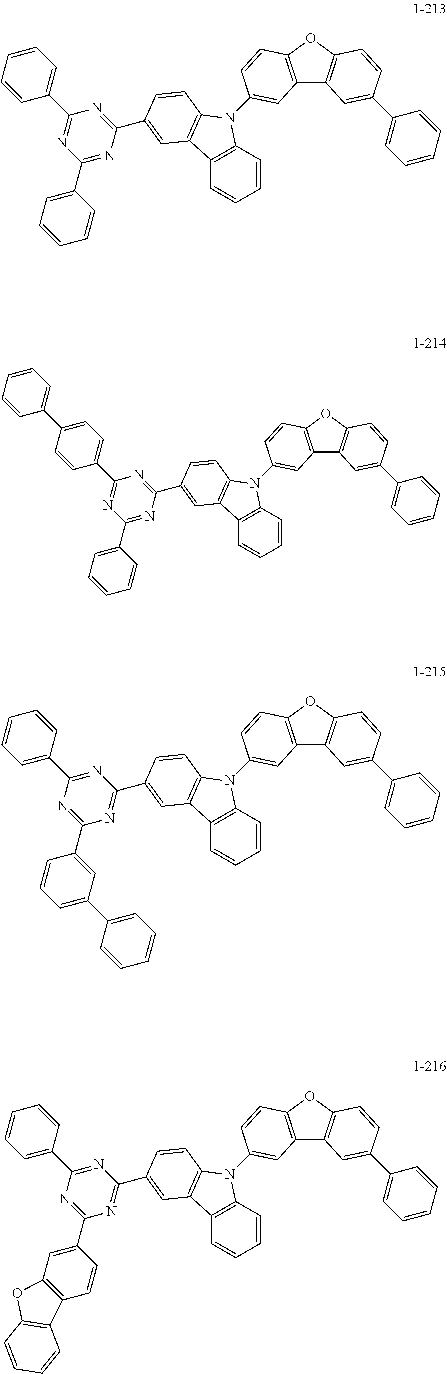 [SMILES: c1ccc(-c2ccc3oc4ccc(-n5c6ccccc6c6cc(-c7nc(-c8ccccc8)nc(-c8ccccc8)n7)ccc65)cc4c3c2)cc1 ; c1ccc(-c2ccc(-c3nc(-c4ccccc4)nc(-c4ccc5c(c4)c4ccccc4n5-c4ccc5oc6ccc(-c7ccccc7)cc6c5c4)n3)cc2)cc1 ; c1ccc(-c2cccc(-c3nc(-c4ccccc4)nc(-c4ccc5c(c4)c4ccccc4n5-c4ccc5oc6ccc(-c7ccccc7)cc6c5c4)n3)c2)cc1 ; c1ccc(-c2ccc3oc4ccc(-n5c6ccccc6c6cc(-c7nc(-c8ccccc8)nc(-c8ccc9c(c8)oc8ccccc89)n7)ccc65)cc4c3c2)cc1]

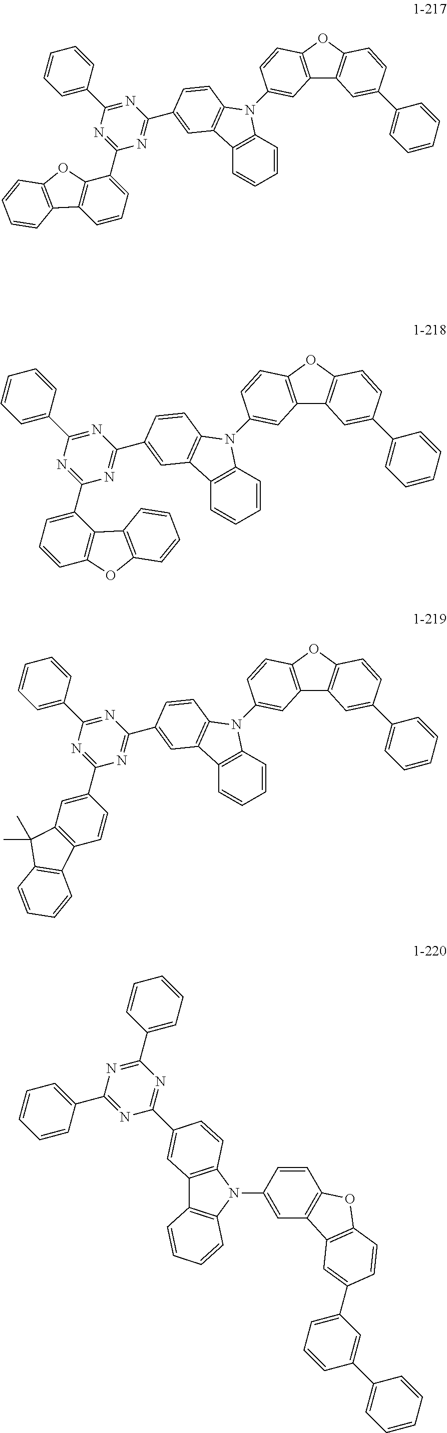 [SMILES: c1ccc(-c2ccc3oc4ccc(-n5c6ccccc6c6cc(-c7nc(-c8ccccc8)nc(-c8cccc9c8oc8ccccc89)n7)ccc65)cc4c3c2)cc1 ; c1ccc(-c2ccc3oc4ccc(-n5c6ccccc6c6cc(-c7nc(-c8ccccc8)nc(-c8cccc9oc%10ccccc%10c89)n7)ccc65)cc4c3c2)cc1 ; CC1(C)c2ccccc2-c2ccc(-c3nc(-c4ccccc4)nc(-c4ccc5c(c4)c4ccccc4n5-c4ccc5oc6ccc(-c7ccccc7)cc6c5c4)n3)cc21 ; c1ccc(-c2cccc(-c3ccc4oc5ccc(-n6c7ccccc7c7cc(-c8nc(-c9ccccc9)nc(-c9ccccc9)n8)ccc76)cc5c4c3)c2)cc1]

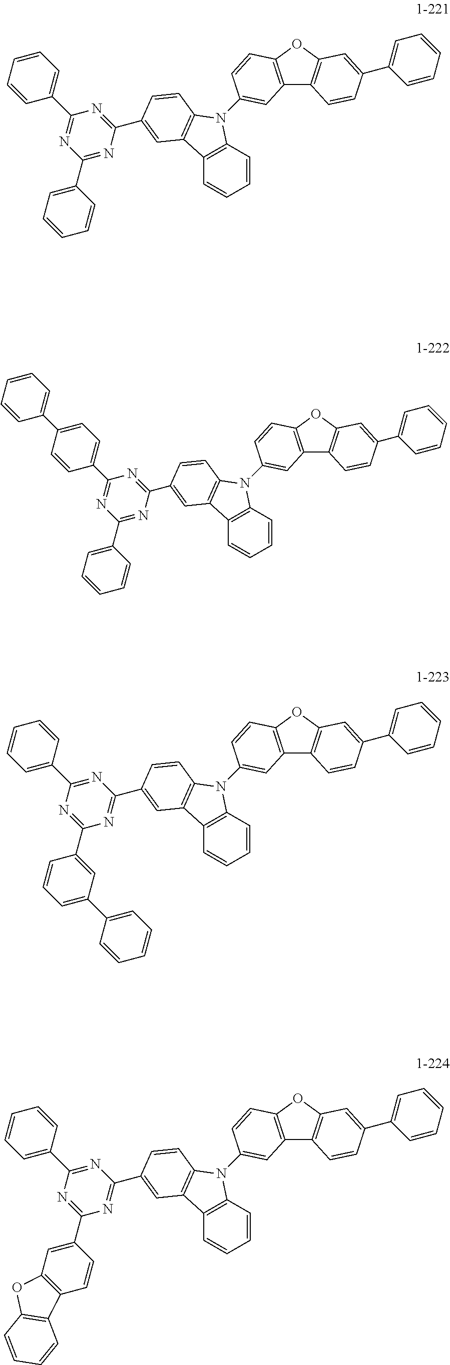 [SMILES: c1ccc(-c2ccc3c(c2)oc2ccc(-n4c5ccccc5c5cc(-c6nc(-c7ccccc7)nc(-c7ccccc7)n6)ccc54)cc23)cc1 ; c1ccc(-c2ccc(-c3nc(-c4ccccc4)nc(-c4ccc5c(c4)c4ccccc4n5-c4ccc5oc6cc(-c7ccccc7)ccc6c5c4)n3)cc2)cc1 ; c1ccc(-c2cccc(-c3nc(-c4ccccc4)nc(-c4ccc5c(c4)c4ccccc4n5-c4ccc5oc6cc(-c7ccccc7)ccc6c5c4)n3)c2)cc1 ; c1ccc(-c2ccc3c(c2)oc2ccc(-n4c5ccccc5c5cc(-c6nc(-c7ccccc7)nc(-c7ccc8c(c7)oc7ccccc78)n6)ccc54)cc23)cc1]

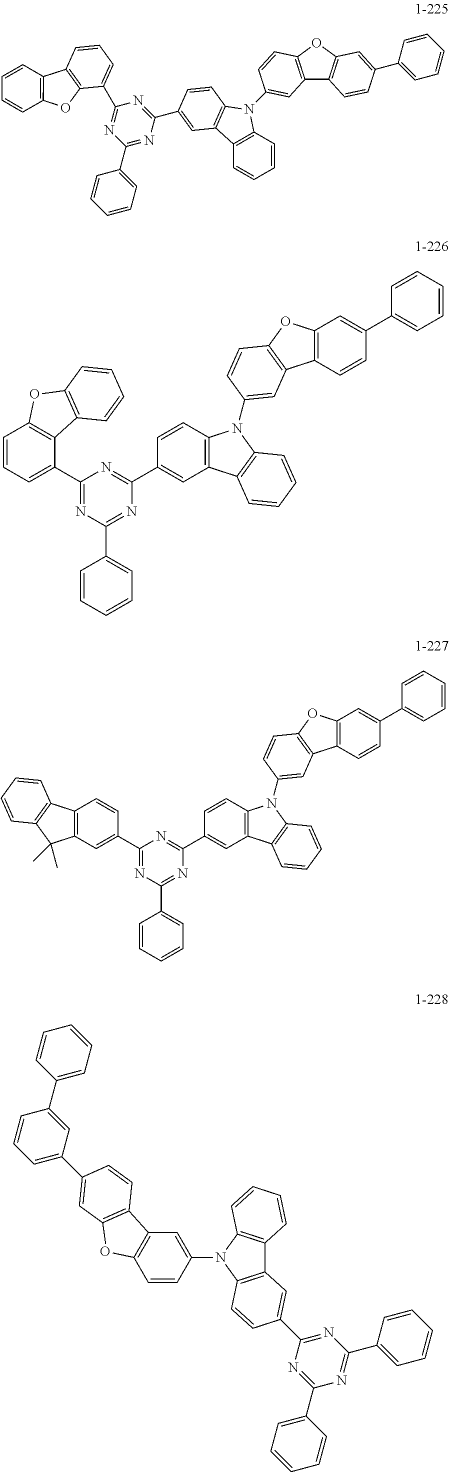 [SMILES: c1ccc(-c2ccc3c(c2)oc2ccc(-n4c5ccccc5c5cc(-c6nc(-c7ccccc7)nc(-c7cccc8c7oc7ccccc78)n6)ccc54)cc23)cc1 ; c1ccc(-c2ccc3c(c2)oc2ccc(-n4c5ccccc5c5cc(-c6nc(-c7ccccc7)nc(-c7cccc8oc9ccccc9c78)n6)ccc54)cc23)cc1 ; CC1(C)c2ccccc2-c2ccc(-c3nc(-c4ccccc4)nc(-c4ccc5c(c4)c4ccccc4n5-c4ccc5oc6cc(-c7ccccc7)ccc6c5c4)n3)cc21 ; c1ccc(-c2cccc(-c3ccc4c(c3)oc3ccc(-n5c6ccccc6c6cc(-c7nc(-c8ccccc8)nc(-c8ccccc8)n7)ccc65)cc34)c2)cc1]

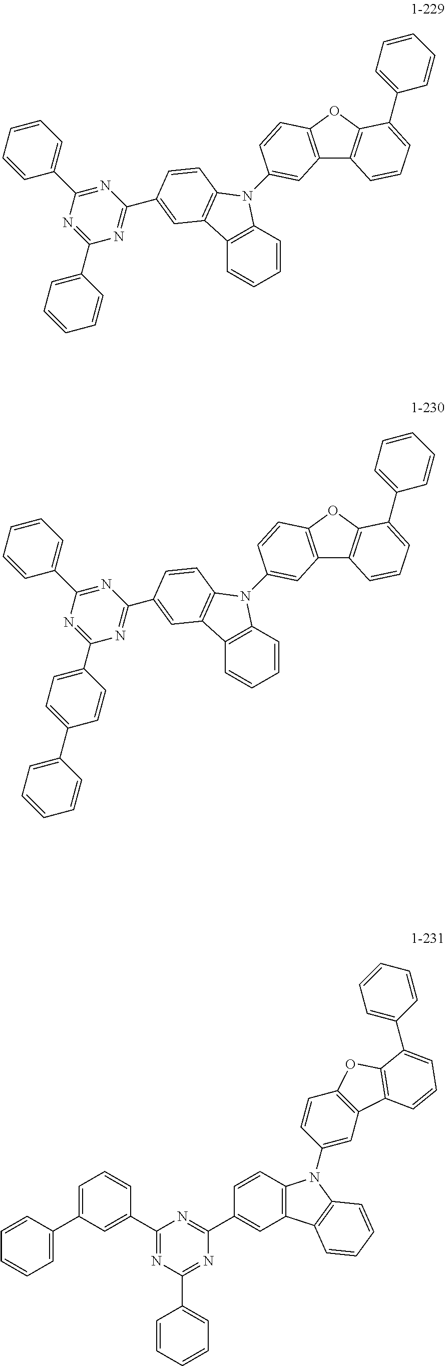 [SMILES: c1ccc(-c2nc(-c3ccccc3)nc(-c3ccc4c(c3)c3ccccc3n4-c3ccc4oc5c(-c6ccccc6)cccc5c4c3)n2)cc1 ; c1ccc(-c2ccc(-c3nc(-c4ccccc4)nc(-c4ccc5c(c4)c4ccccc4n5-c4ccc5oc6c(-c7ccccc7)cccc6c5c4)n3)cc2)cc1 ; c1ccc(-c2cccc(-c3nc(-c4ccccc4)nc(-c4ccc5c(c4)c4ccccc4n5-c4ccc5oc6c(-c7ccccc7)cccc6c5c4)n3)c2)cc1]

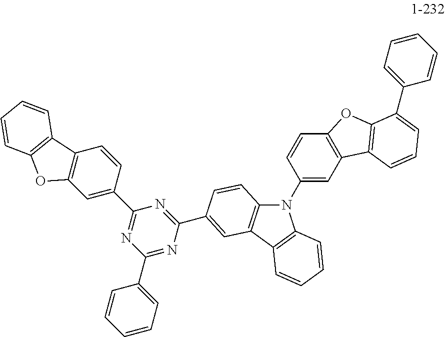 [SMILES: c1ccc(-c2nc(-c3ccc4c(c3)oc3ccccc34)nc(-c3ccc4c(c3)c3ccccc3n4-c3ccc4oc5c(-c6ccccc6)cccc5c4c3)n2)cc1]

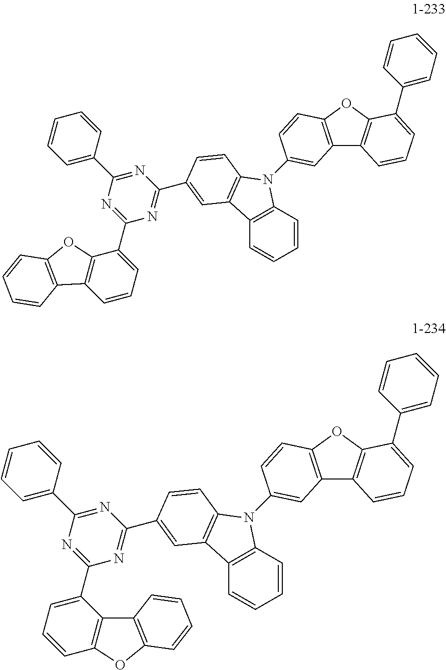 [SMILES: c1ccc(-c2nc(-c3ccc4c(c3)c3ccccc3n4-c3ccc4oc5c(-c6ccccc6)cccc5c4c3)nc(-c3cccc4c3oc3ccccc34)n2)cc1 ; c1ccc(-c2nc(-c3ccc4c(c3)c3ccccc3n4-c3ccc4oc5c(-c6ccccc6)cccc5c4c3)nc(-c3cccc4oc5ccccc5c34)n2)cc1]

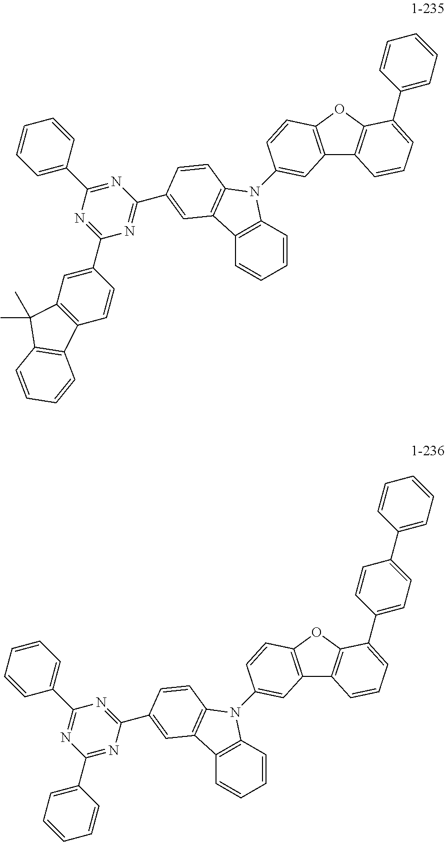 [SMILES: CC1(C)c2ccccc2-c2ccc(-c3nc(-c4ccccc4)nc(-c4ccc5c(c4)c4ccccc4n5-c4ccc5oc6c(-c7ccccc7)cccc6c5c4)n3)cc21 ; c1ccc(-c2ccc(-c3cccc4c3oc3ccc(-n5c6ccccc6c6cc(-c7nc(-c8ccccc8)nc(-c8ccccc8)n7)ccc65)cc34)cc2)cc1]

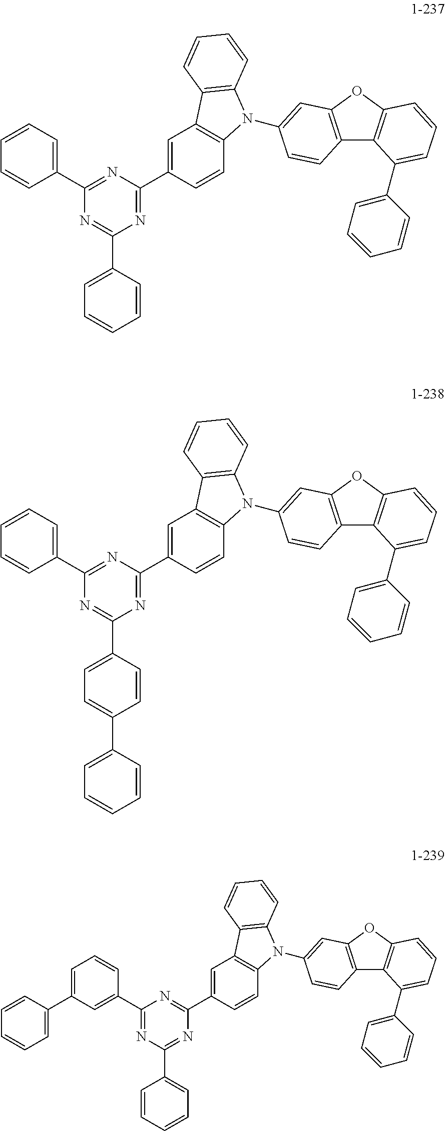 [SMILES: c1ccc(-c2nc(-c3ccccc3)nc(-c3ccc4c(c3)c3ccccc3n4-c3ccc4c(c3)oc3cccc(-c5ccccc5)c34)n2)cc1 ; c1ccc(-c2ccc(-c3nc(-c4ccccc4)nc(-c4ccc5c(c4)c4ccccc4n5-c4ccc5c(c4)oc4cccc(-c6ccccc6)c45)n3)cc2)cc1 ; c1ccc(-c2cccc(-c3nc(-c4ccccc4)nc(-c4ccc5c(c4)c4ccccc4n5-c4ccc5c(c4)oc4cccc(-c6ccccc6)c45)n3)c2)cc1]

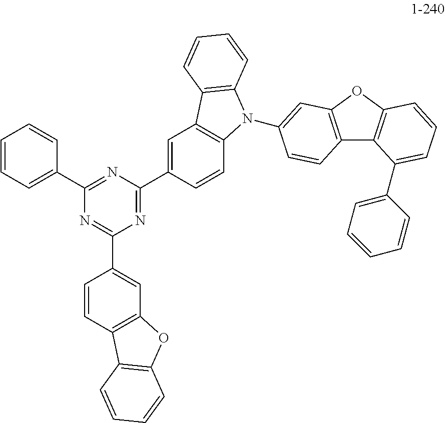 [SMILES: c1ccc(-c2nc(-c3ccc4c(c3)oc3ccccc34)nc(-c3ccc4c(c3)c3ccccc3n4-c3ccc4c(c3)oc3cccc(-c5ccccc5)c34)n2)cc1]

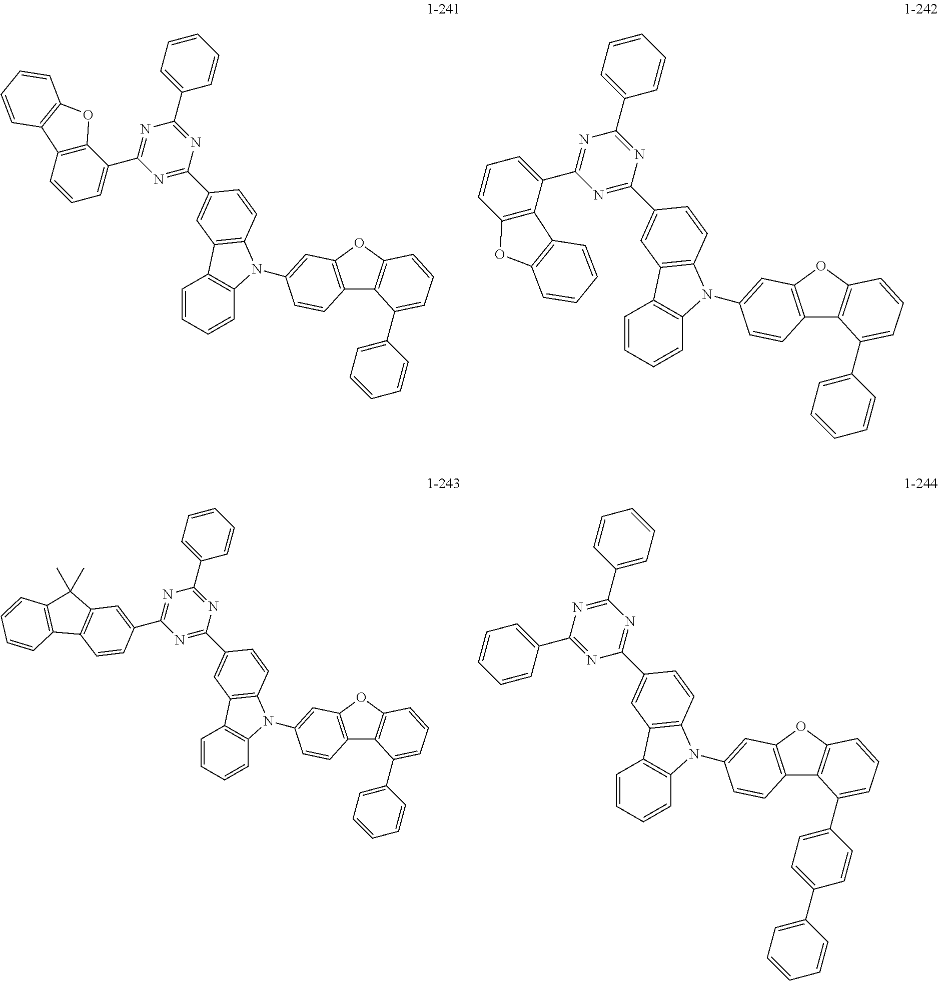 [SMILES: c1ccc(-c2nc(-c3ccc4c(c3)c3ccccc3n4-c3ccc4c(c3)oc3cccc(-c5ccccc5)c34)nc(-c3cccc4c3oc3ccccc34)n2)cc1 ; c1ccc(-c2nc(-c3ccc4c(c3)c3ccccc3n4-c3ccc4c(c3)oc3cccc(-c5ccccc5)c34)nc(-c3cccc4oc5ccccc5c34)n2)cc1 ; CC1(C)c2ccccc2-c2ccc(-c3nc(-c4ccccc4)nc(-c4ccc5c(c4)c4ccccc4n5-c4ccc5c(c4)oc4cccc(-c6ccccc6)c45)n3)cc21 ; c1ccc(-c2ccc(-c3cccc4oc5cc(-n6c7ccccc7c7cc(-c8nc(-c9ccccc9)nc(-c9ccccc9)n8)ccc76)ccc5c34)cc2)cc1]

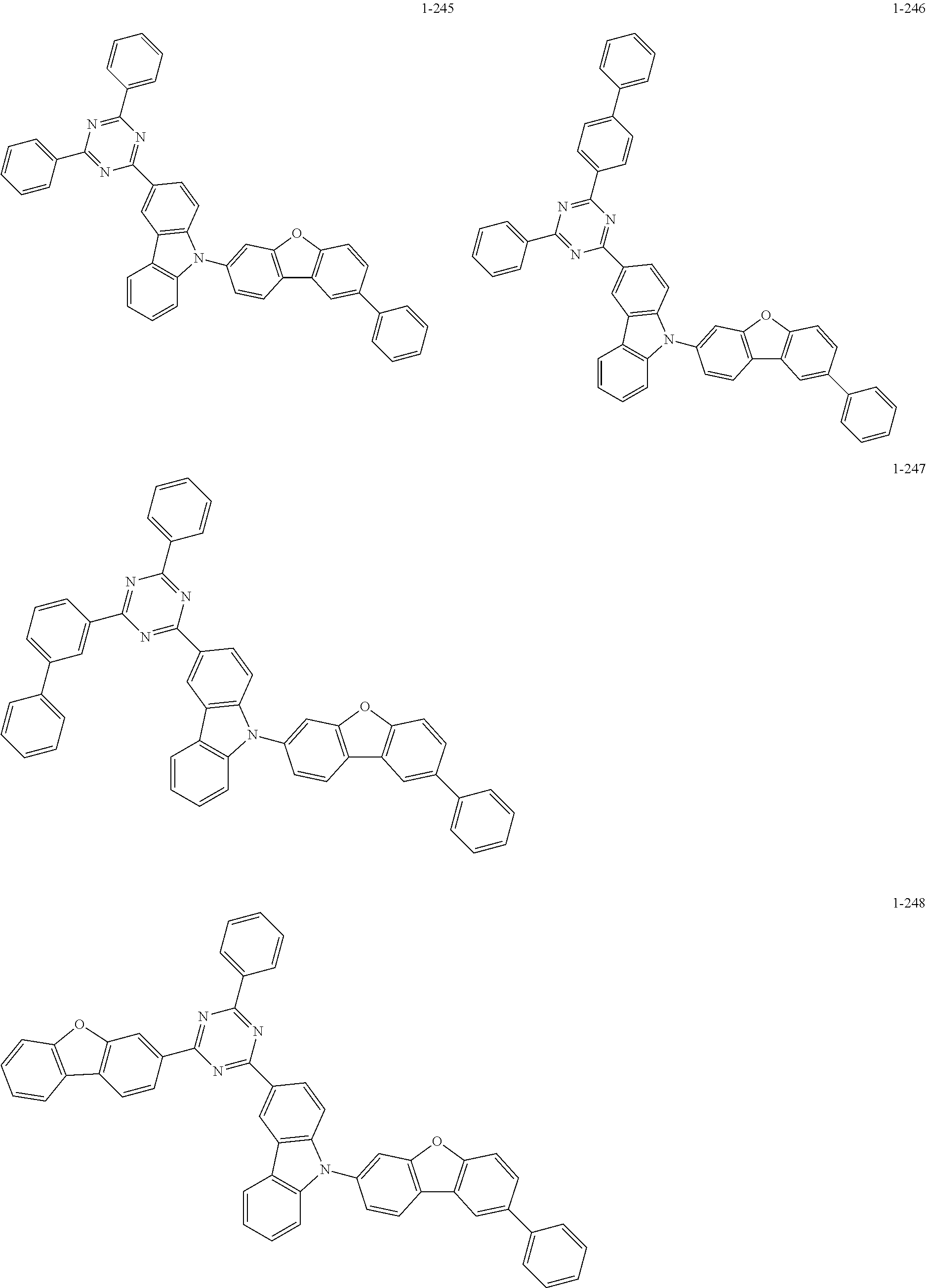 [SMILES: c1ccc(-c2ccc3oc4cc(-n5c6ccccc6c6cc(-c7nc(-c8ccccc8)nc(-c8ccccc8)n7)ccc65)ccc4c3c2)cc1 ; c1ccc(-c2ccc(-c3nc(-c4ccccc4)nc(-c4ccc5c(c4)c4ccccc4n5-c4ccc5c(c4)oc4ccc(-c6ccccc6)cc45)n3)cc2)cc1 ; c1ccc(-c2cccc(-c3nc(-c4ccccc4)nc(-c4ccc5c(c4)c4ccccc4n5-c4ccc5c(c4)oc4ccc(-c6ccccc6)cc45)n3)c2)cc1 ; c1ccc(-c2ccc3oc4cc(-n5c6ccccc6c6cc(-c7nc(-c8ccccc8)nc(-c8ccc9c(c8)oc8ccccc89)n7)ccc65)ccc4c3c2)cc1]

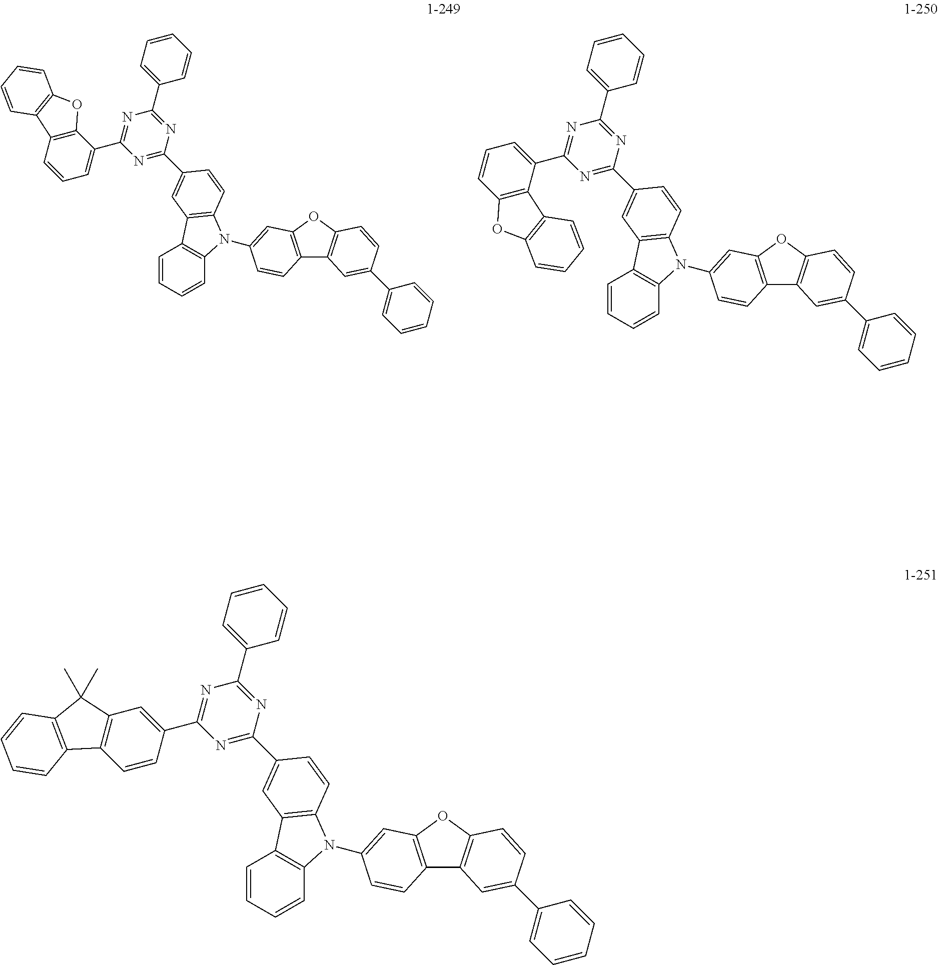 [SMILES: c1ccc(-c2ccc3oc4cc(-n5c6ccccc6c6cc(-c7nc(-c8ccccc8)nc(-c8cccc9c8oc8ccccc89)n7)ccc65)ccc4c3c2)cc1 ; c1ccc(-c2ccc3oc4cc(-n5c6ccccc6c6cc(-c7nc(-c8ccccc8)nc(-c8cccc9oc%10ccccc%10c89)n7)ccc65)ccc4c3c2)cc1 ; CC1(C)c2ccccc2-c2ccc(-c3nc(-c4ccccc4)nc(-c4ccc5c(c4)c4ccccc4n5-c4ccc5c(c4)oc4ccc(-c6ccccc6)cc45)n3)cc21]

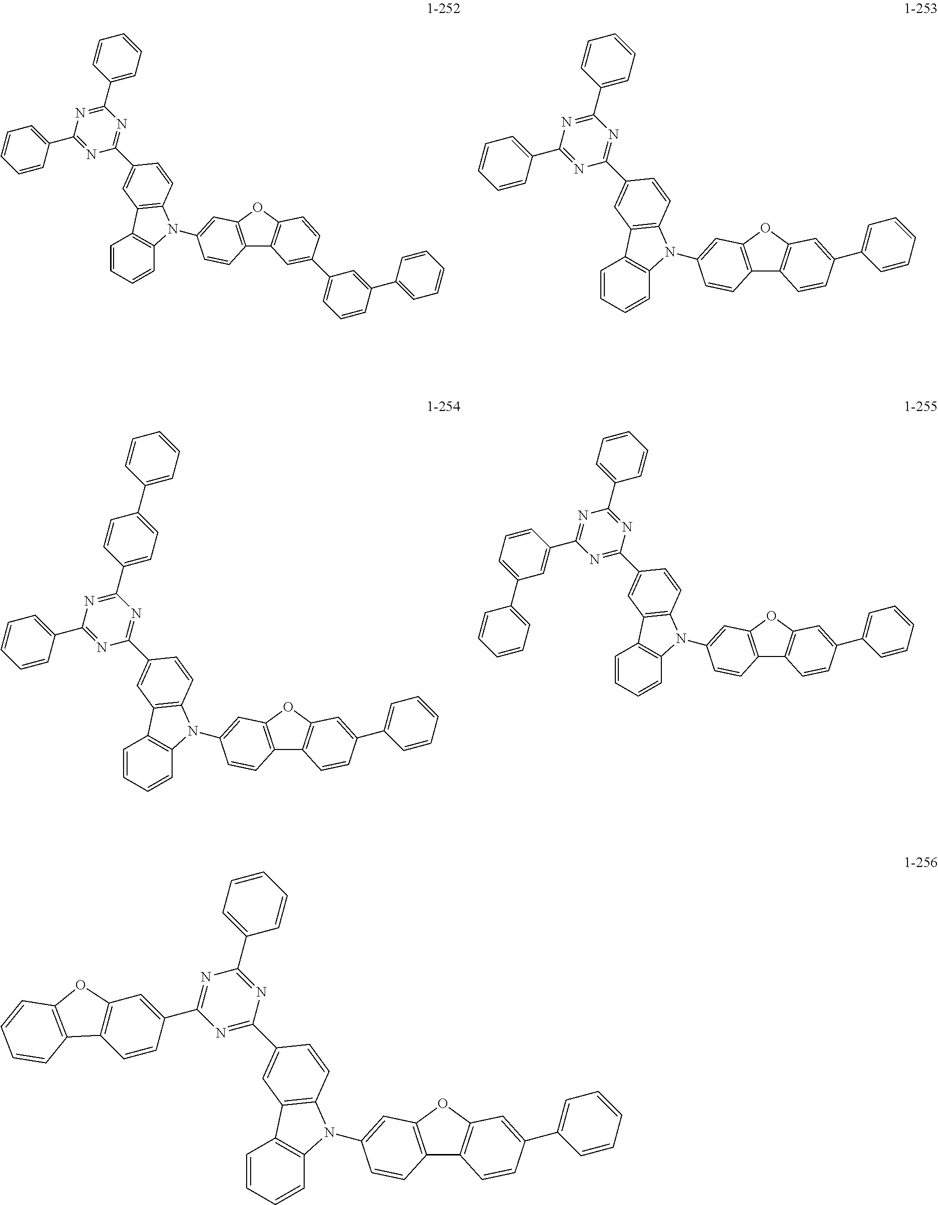 [SMILES: c1ccc(-c2cccc(-c3ccc4oc5cc(-n6c7ccccc7c7cc(-c8nc(-c9ccccc9)nc(-c9ccccc9)n8)ccc76)ccc5c4c3)c2)cc1 ; c1ccc(-c2ccc3c(c2)oc2cc(-n4c5ccccc5c5cc(-c6nc(-c7ccccc7)nc(-c7ccccc7)n6)ccc54)ccc23)cc1 ; c1ccc(-c2ccc(-c3nc(-c4ccccc4)nc(-c4ccc5c(c4)c4ccccc4n5-c4ccc5c(c4)oc4cc(-c6ccccc6)ccc45)n3)cc2)cc1 ; c1ccc(-c2cccc(-c3nc(-c4ccccc4)nc(-c4ccc5c(c4)c4ccccc4n5-c4ccc5c(c4)oc4cc(-c6ccccc6)ccc45)n3)c2)cc1 ; c1ccc(-c2ccc3c(c2)oc2cc(-n4c5ccccc5c5cc(-c6nc(-c7ccccc7)nc(-c7ccc8c(c7)oc7ccccc78)n6)ccc54)ccc23)cc1]

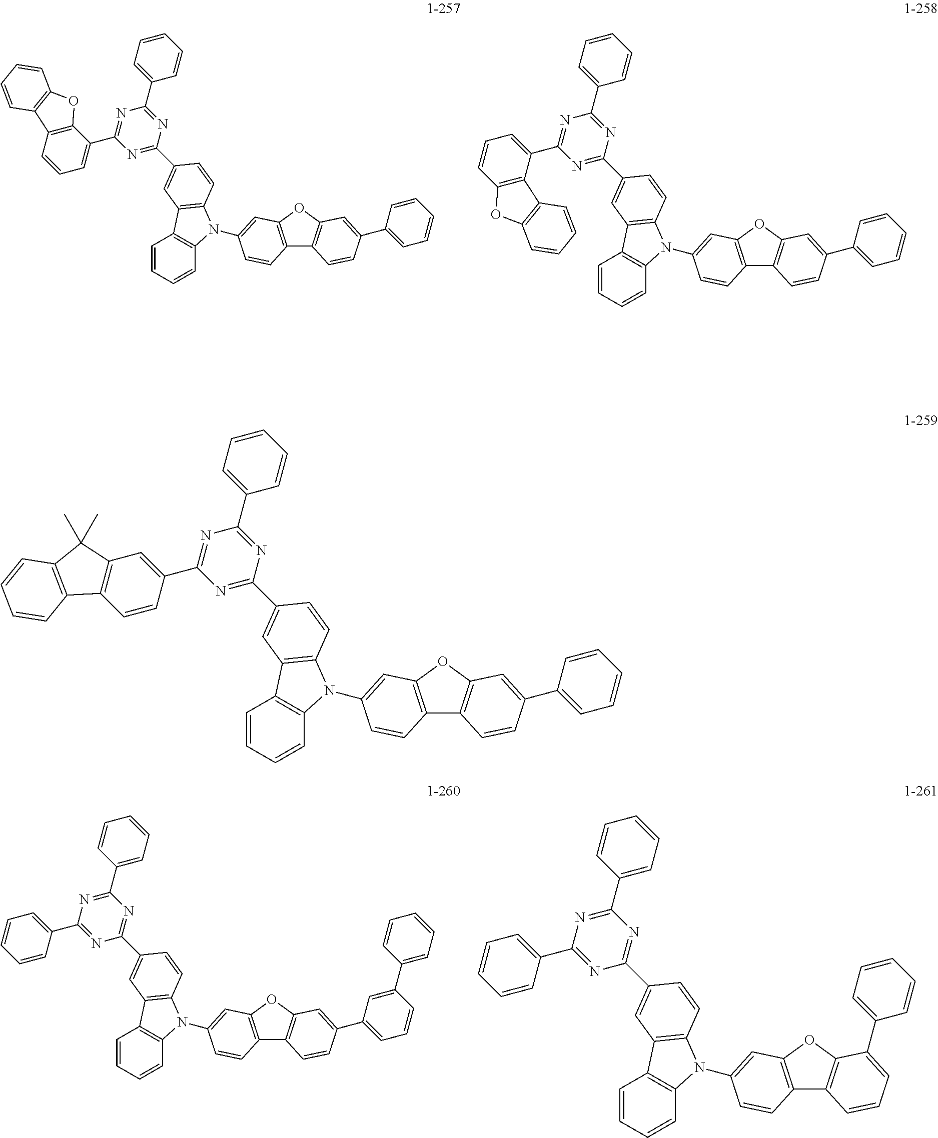 [SMILES: c1ccc(-c2ccc3c(c2)oc2cc(-n4c5ccccc5c5cc(-c6nc(-c7ccccc7)nc(-c7cccc8c7oc7ccccc78)n6)ccc54)ccc23)cc1 ; c1ccc(-c2ccc3c(c2)oc2cc(-n4c5ccccc5c5cc(-c6nc(-c7ccccc7)nc(-c7cccc8oc9ccccc9c78)n6)ccc54)ccc23)cc1 ; CC1(C)c2ccccc2-c2ccc(-c3nc(-c4ccccc4)nc(-c4ccc5c(c4)c4ccccc4n5-c4ccc5c(c4)oc4cc(-c6ccccc6)ccc45)n3)cc21 ; c1ccc(-c2cccc(-c3ccc4c(c3)oc3cc(-n5c6ccccc6c6cc(-c7nc(-c8ccccc8)nc(-c8ccccc8)n7)ccc65)ccc34)c2)cc1 ; c1ccc(-c2nc(-c3ccccc3)nc(-c3ccc4c(c3)c3ccccc3n4-c3ccc4c(c3)oc3c(-c5ccccc5)cccc34)n2)cc1]

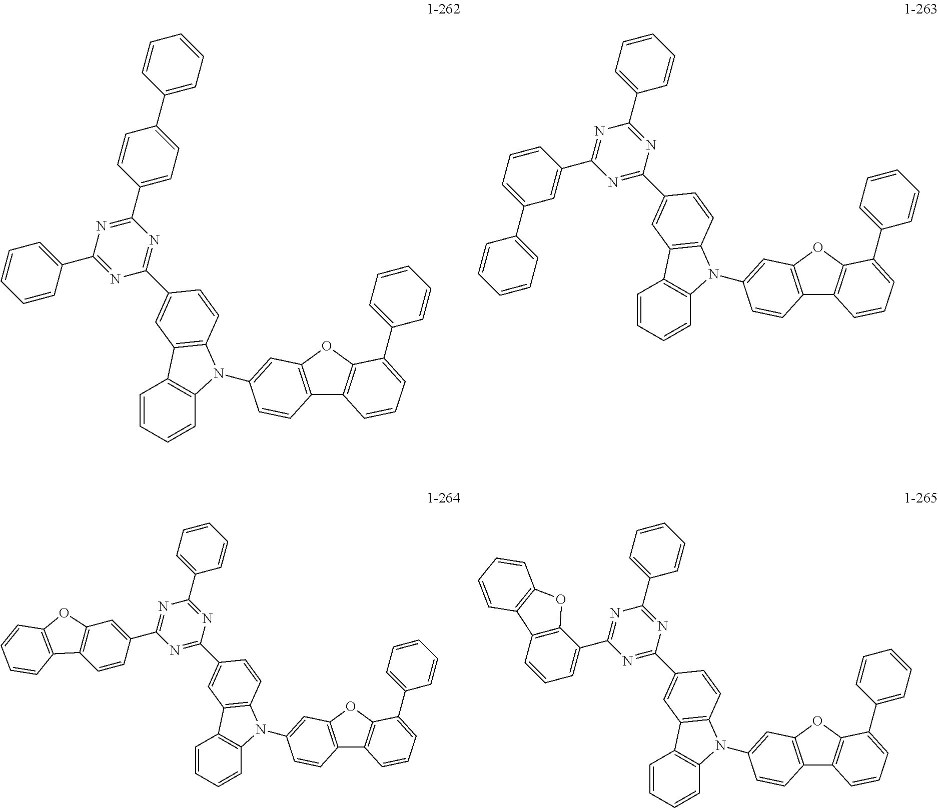 [SMILES: c1ccc(-c2ccc(-c3nc(-c4ccccc4)nc(-c4ccc5c(c4)c4ccccc4n5-c4ccc5c(c4)oc4c(-c6ccccc6)cccc45)n3)cc2)cc1 ; c1ccc(-c2cccc(-c3nc(-c4ccccc4)nc(-c4ccc5c(c4)c4ccccc4n5-c4ccc5c(c4)oc4c(-c6ccccc6)cccc45)n3)c2)cc1 ; c1ccc(-c2nc(-c3ccc4c(c3)oc3ccccc34)nc(-c3ccc4c(c3)c3ccccc3n4-c3ccc4c(c3)oc3c(-c5ccccc5)cccc34)n2)cc1 ; c1ccc(-c2nc(-c3ccc4c(c3)c3ccccc3n4-c3ccc4c(c3)oc3c(-c5ccccc5)cccc34)nc(-c3cccc4c3oc3ccccc34)n2)cc1]

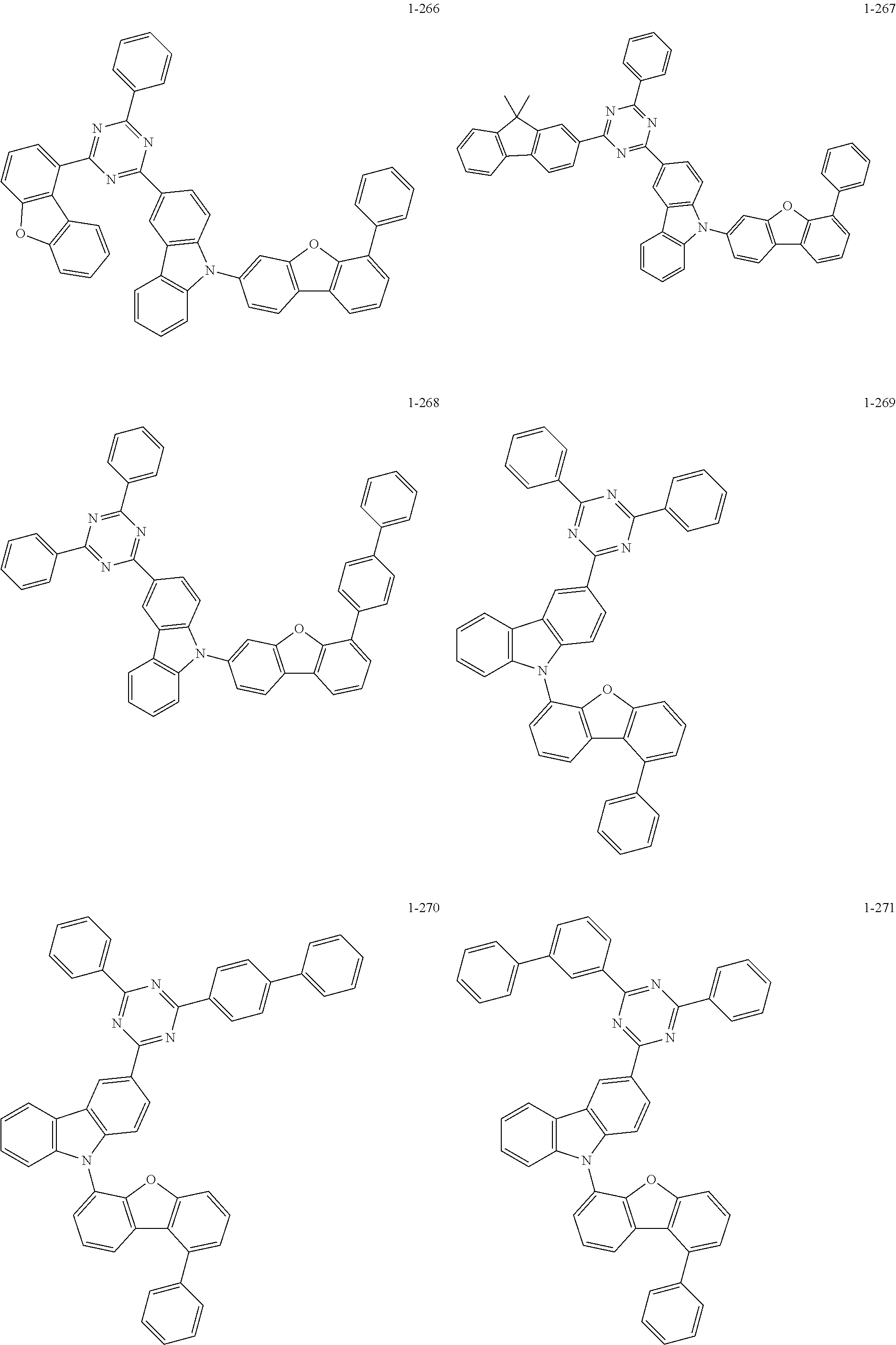

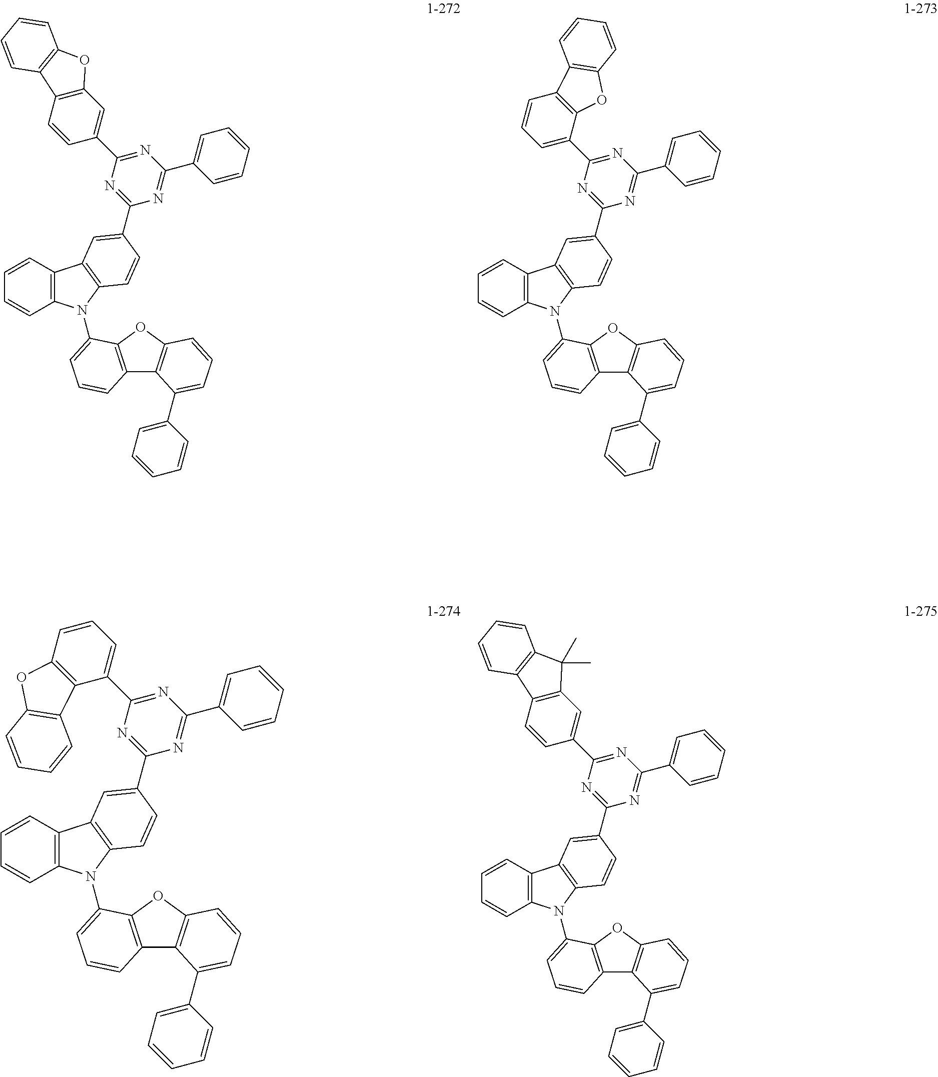 [SMILES: c1ccc(-c2nc(-c3ccc4c(c3)oc3ccccc34)nc(-c3ccc4c(c3)c3ccccc3n4-c3cccc4c3oc3cccc(-c5ccccc5)c34)n2)cc1 ; c1ccc(-c2nc(-c3ccc4c(c3)c3ccccc3n4-c3cccc4c3oc3cccc(-c5ccccc5)c34)nc(-c3cccc4c3oc3ccccc34)n2)cc1 ; c1ccc(-c2nc(-c3ccc4c(c3)c3ccccc3n4-c3cccc4c3oc3cccc(-c5ccccc5)c34)nc(-c3cccc4oc5ccccc5c34)n2)cc1 ; CC1(C)c2ccccc2-c2ccc(-c3nc(-c4ccccc4)nc(-c4ccc5c(c4)c4ccccc4n5-c4cccc5c4oc4cccc(-c6ccccc6)c45)n3)cc21]

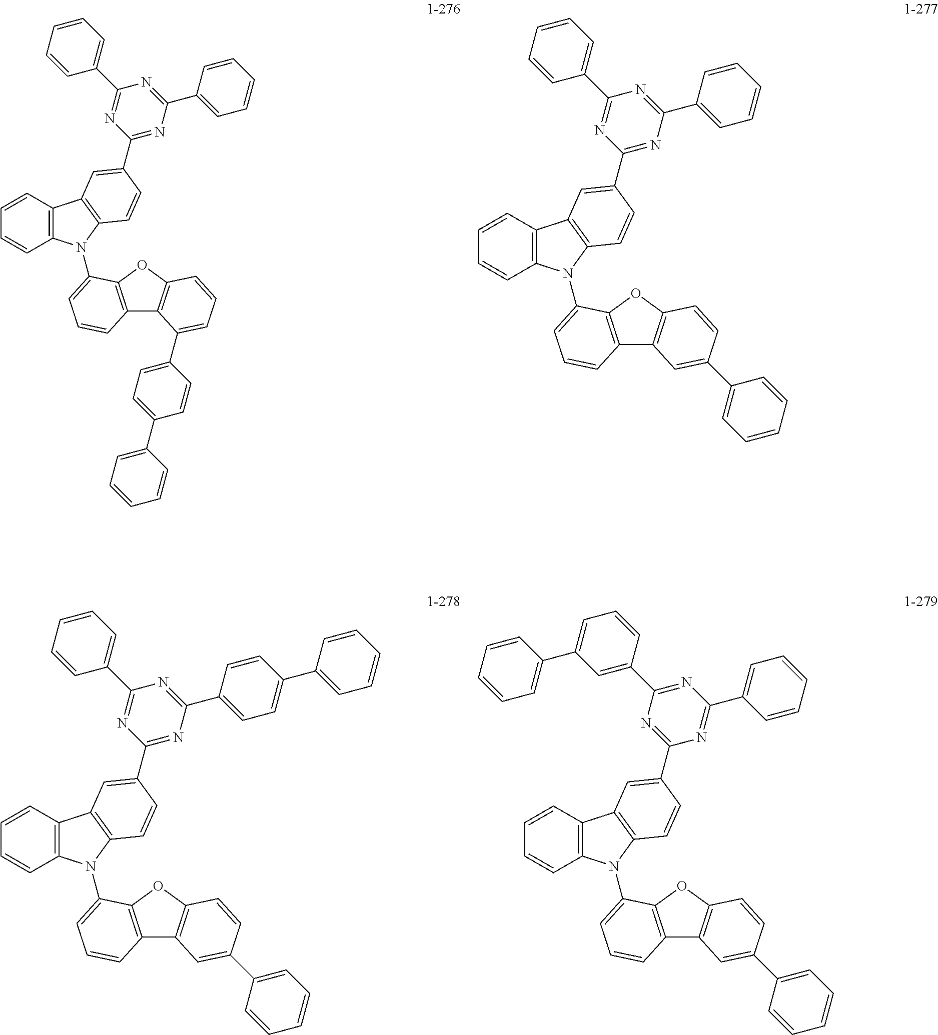 [SMILES: c1ccc(-c2ccc(-c3cccc4oc5c(-n6c7ccccc7c7cc(-c8nc(-c9ccccc9)nc(-c9ccccc9)n8)ccc76)cccc5c34)cc2)cc1 ; c1ccc(-c2ccc3oc4c(-n5c6ccccc6c6cc(-c7nc(-c8ccccc8)nc(-c8ccccc8)n7)ccc65)cccc4c3c2)cc1 ; c1ccc(-c2ccc(-c3nc(-c4ccccc4)nc(-c4ccc5c(c4)c4ccccc4n5-c4cccc5c4oc4ccc(-c6ccccc6)cc45)n3)cc2)cc1 ; c1ccc(-c2cccc(-c3nc(-c4ccccc4)nc(-c4ccc5c(c4)c4ccccc4n5-c4cccc5c4oc4ccc(-c6ccccc6)cc45)n3)c2)cc1]

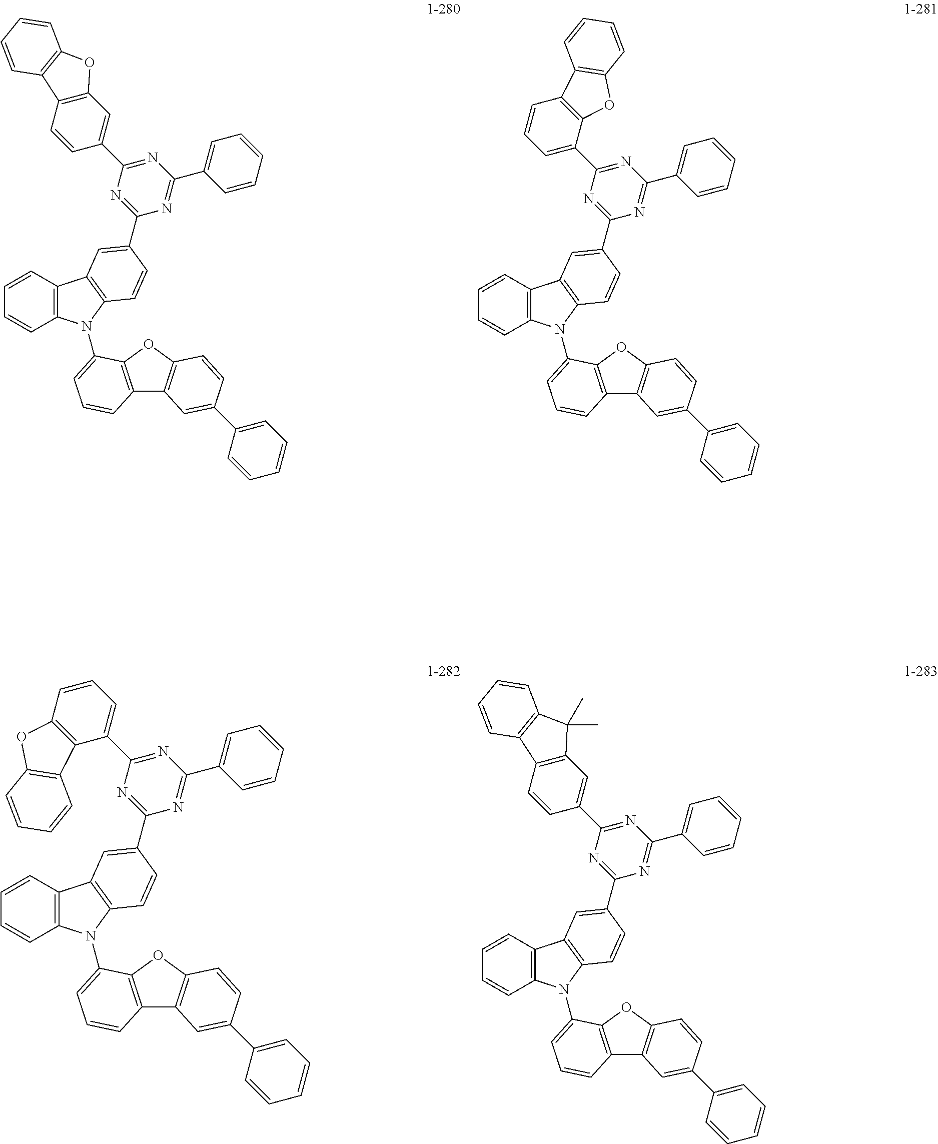 [SMILES: c1ccc(-c2ccc3oc4c(-n5c6ccccc6c6cc(-c7nc(-c8ccccc8)nc(-c8ccc9c(c8)oc8ccccc89)n7)ccc65)cccc4c3c2)cc1 ; c1ccc(-c2ccc3oc4c(-n5c6ccccc6c6cc(-c7nc(-c8ccccc8)nc(-c8cccc9c8oc8ccccc89)n7)ccc65)cccc4c3c2)cc1 ; c1ccc(-c2ccc3oc4c(-n5c6ccccc6c6cc(-c7nc(-c8ccccc8)nc(-c8cccc9oc%10ccccc%10c89)n7)ccc65)cccc4c3c2)cc1 ; CC1(C)c2ccccc2-c2ccc(-c3nc(-c4ccccc4)nc(-c4ccc5c(c4)c4ccccc4n5-c4cccc5c4oc4ccc(-c6ccccc6)cc45)n3)cc21]

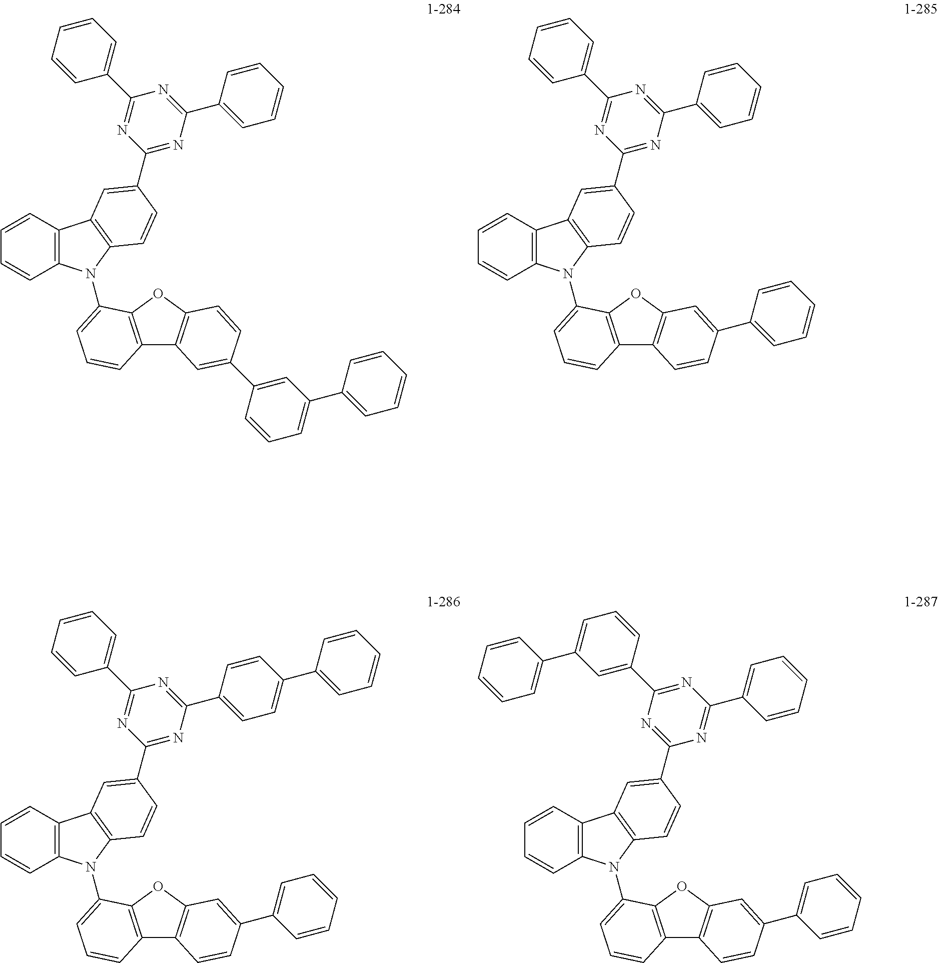 [SMILES: c1ccc(-c2cccc(-c3ccc4oc5c(-n6c7ccccc7c7cc(-c8nc(-c9ccccc9)nc(-c9ccccc9)n8)ccc76)cccc5c4c3)c2)cc1 ; c1ccc(-c2ccc3c(c2)oc2c(-n4c5ccccc5c5cc(-c6nc(-c7ccccc7)nc(-c7ccccc7)n6)ccc54)cccc23)cc1 ; c1ccc(-c2ccc(-c3nc(-c4ccccc4)nc(-c4ccc5c(c4)c4ccccc4n5-c4cccc5c4oc4cc(-c6ccccc6)ccc45)n3)cc2)cc1 ; c1ccc(-c2cccc(-c3nc(-c4ccccc4)nc(-c4ccc5c(c4)c4ccccc4n5-c4cccc5c4oc4cc(-c6ccccc6)ccc45)n3)c2)cc1]

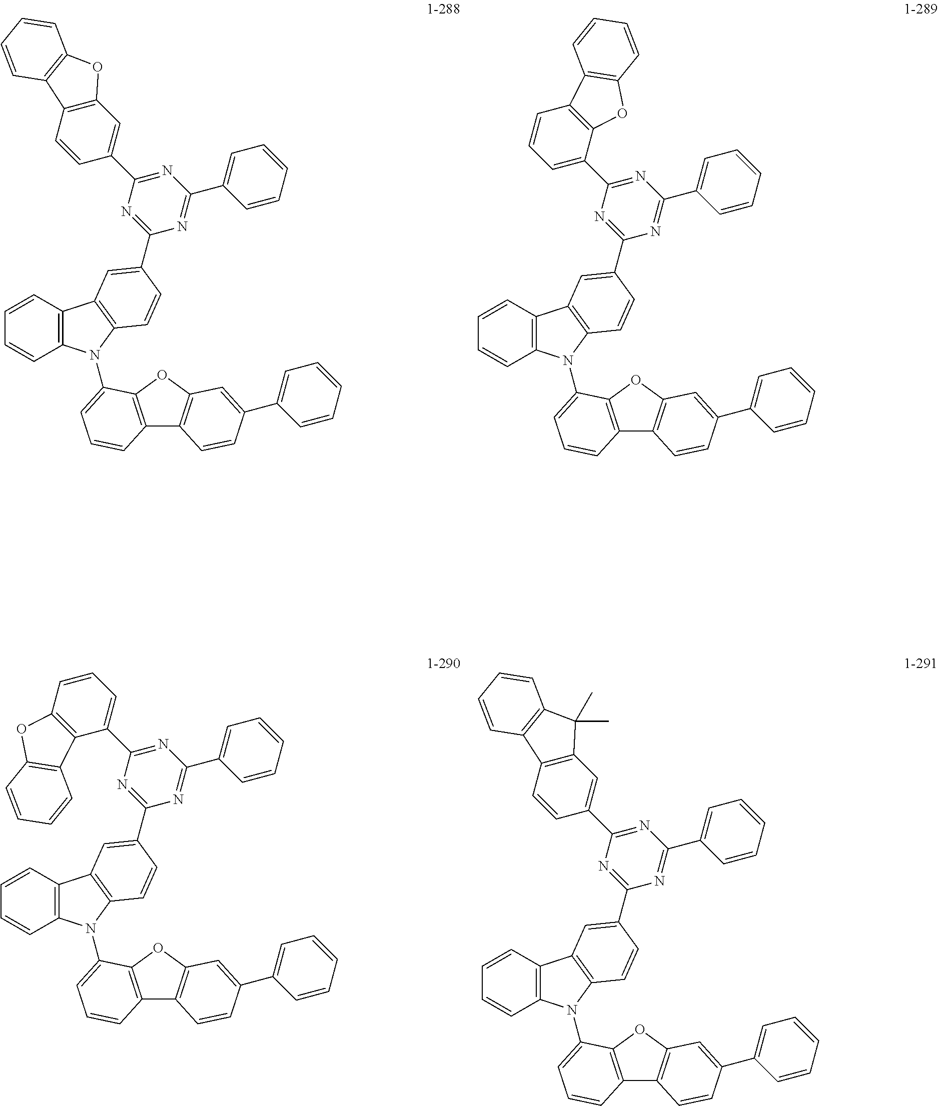 [SMILES: c1ccc(-c2ccc3c(c2)oc2c(-n4c5ccccc5c5cc(-c6nc(-c7ccccc7)nc(-c7ccc8c(c7)oc7ccccc78)n6)ccc54)cccc23)cc1 ; c1ccc(-c2ccc3c(c2)oc2c(-n4c5ccccc5c5cc(-c6nc(-c7ccccc7)nc(-c7cccc8c7oc7ccccc78)n6)ccc54)cccc23)cc1 ; c1ccc(-c2ccc3c(c2)oc2c(-n4c5ccccc5c5cc(-c6nc(-c7ccccc7)nc(-c7cccc8oc9ccccc9c78)n6)ccc54)cccc23)cc1 ; CC1(C)c2ccccc2-c2ccc(-c3nc(-c4ccccc4)nc(-c4ccc5c(c4)c4ccccc4n5-c4cccc5c4oc4cc(-c6ccccc6)ccc45)n3)cc21]

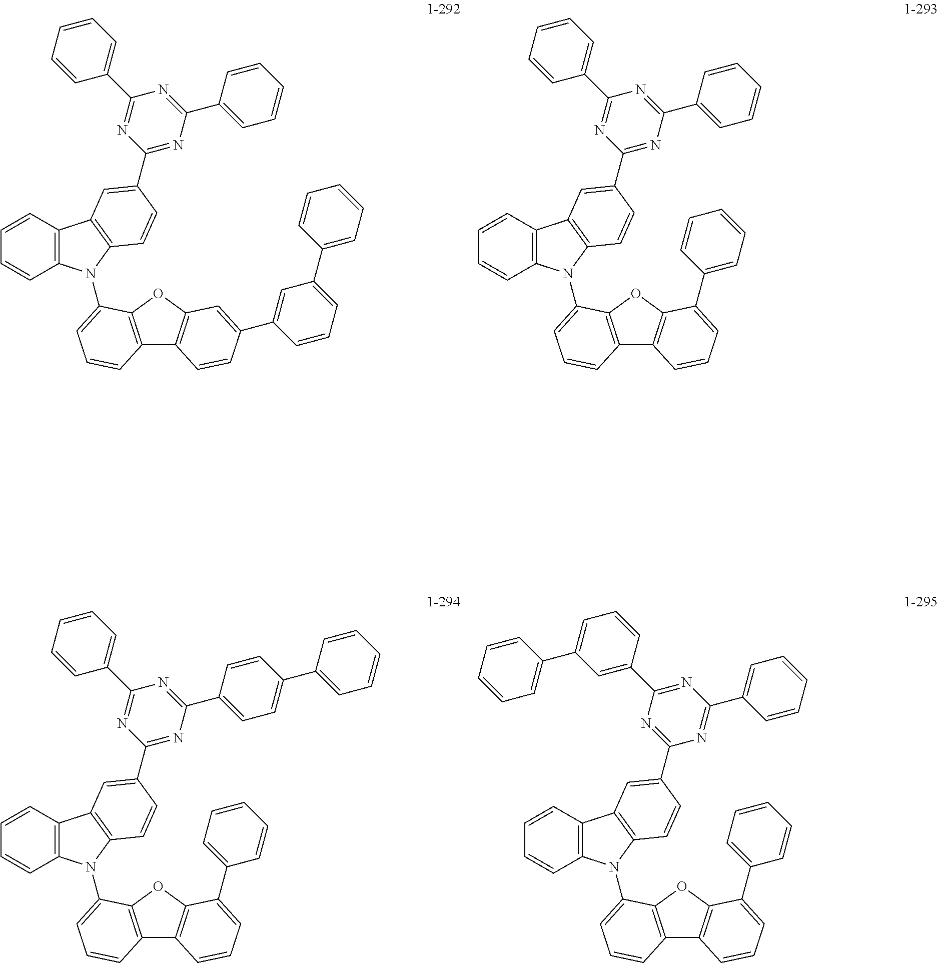 [SMILES: c1ccc(-c2cccc(-c3ccc4c(c3)oc3c(-n5c6ccccc6c6cc(-c7nc(-c8ccccc8)nc(-c8ccccc8)n7)ccc65)cccc34)c2)cc1 ; c1ccc(-c2nc(-c3ccccc3)nc(-c3ccc4c(c3)c3ccccc3n4-c3cccc4c3oc3c(-c5ccccc5)cccc34)n2)cc1 ; c1ccc(-c2ccc(-c3nc(-c4ccccc4)nc(-c4ccc5c(c4)c4ccccc4n5-c4cccc5c4oc4c(-c6ccccc6)cccc45)n3)cc2)cc1 ; c1ccc(-c2cccc(-c3nc(-c4ccccc4)nc(-c4ccc5c(c4)c4ccccc4n5-c4cccc5c4oc4c(-c6ccccc6)cccc45)n3)c2)cc1]

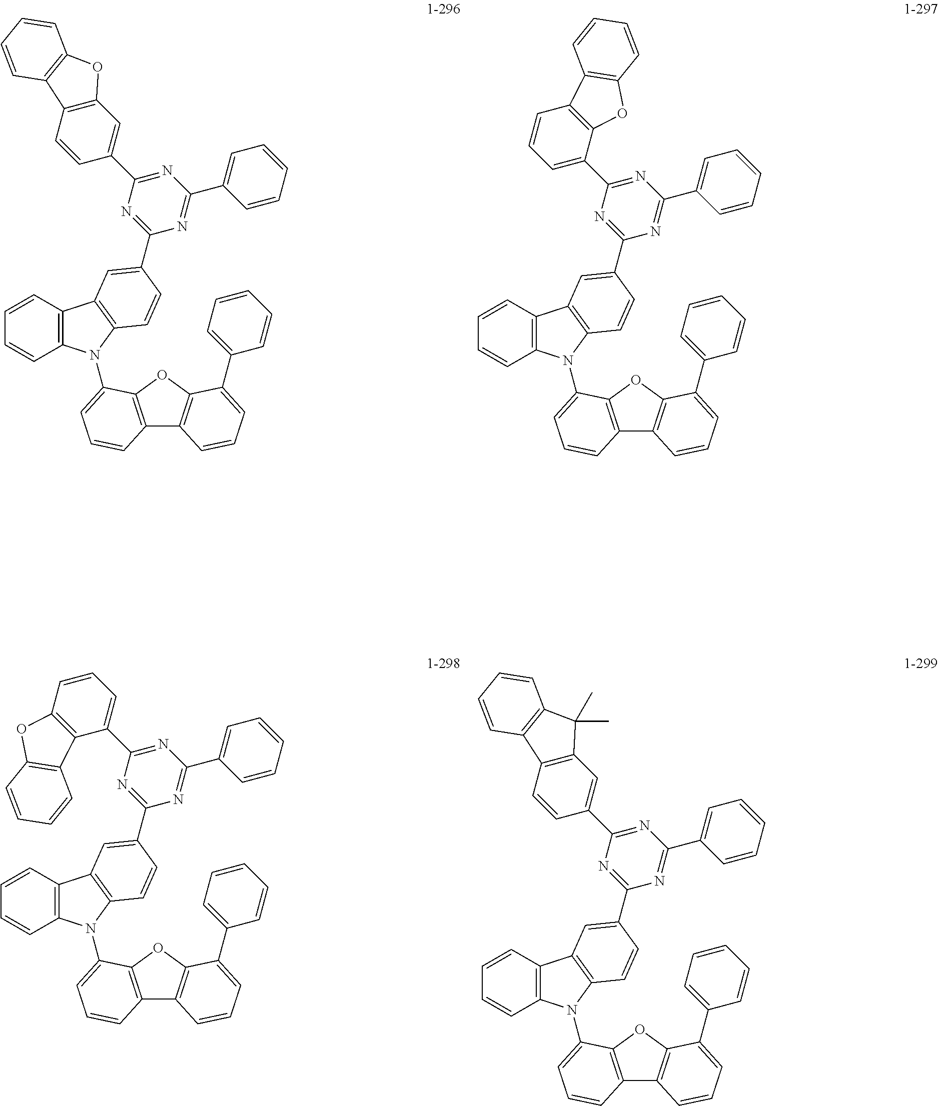 [SMILES: c1ccc(-c2nc(-c3ccc4c(c3)oc3ccccc34)nc(-c3ccc4c(c3)c3ccccc3n4-c3cccc4c3oc3c(-c5ccccc5)cccc34)n2)cc1 ; c1ccc(-c2nc(-c3ccc4c(c3)c3ccccc3n4-c3cccc4c3oc3c(-c5ccccc5)cccc34)nc(-c3cccc4c3oc3ccccc34)n2)cc1 ; c1ccc(-c2nc(-c3ccc4c(c3)c3ccccc3n4-c3cccc4c3oc3c(-c5ccccc5)cccc34)nc(-c3cccc4oc5ccccc5c34)n2)cc1 ; CC1(C)c2ccccc2-c2ccc(-c3nc(-c4ccccc4)nc(-c4ccc5c(c4)c4ccccc4n5-c4cccc5c4oc4c(-c6ccccc6)cccc45)n3)cc21]

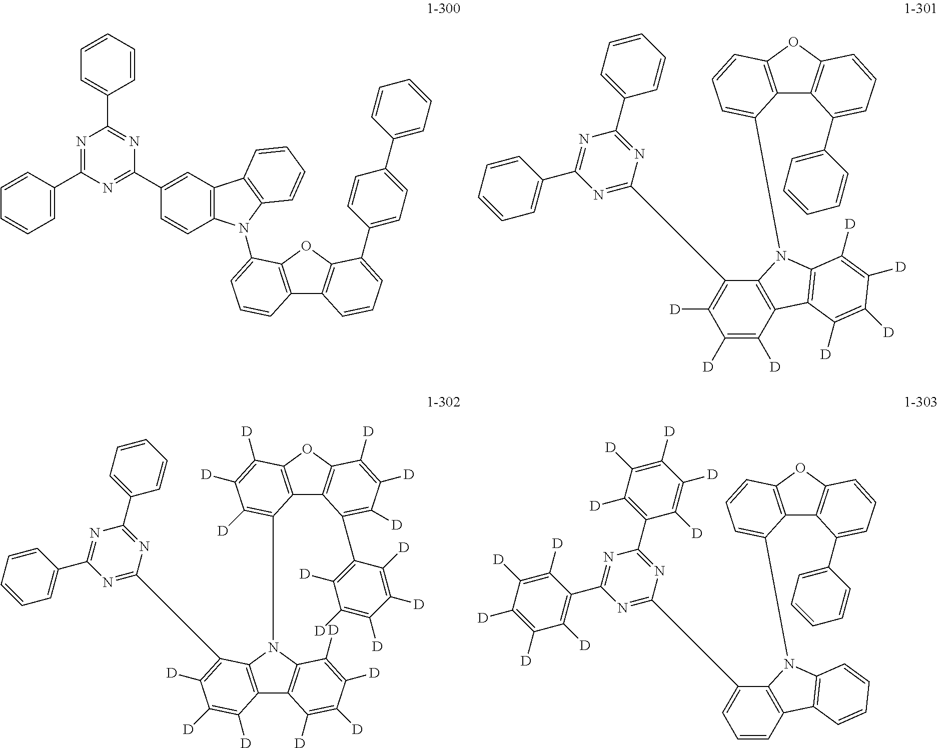

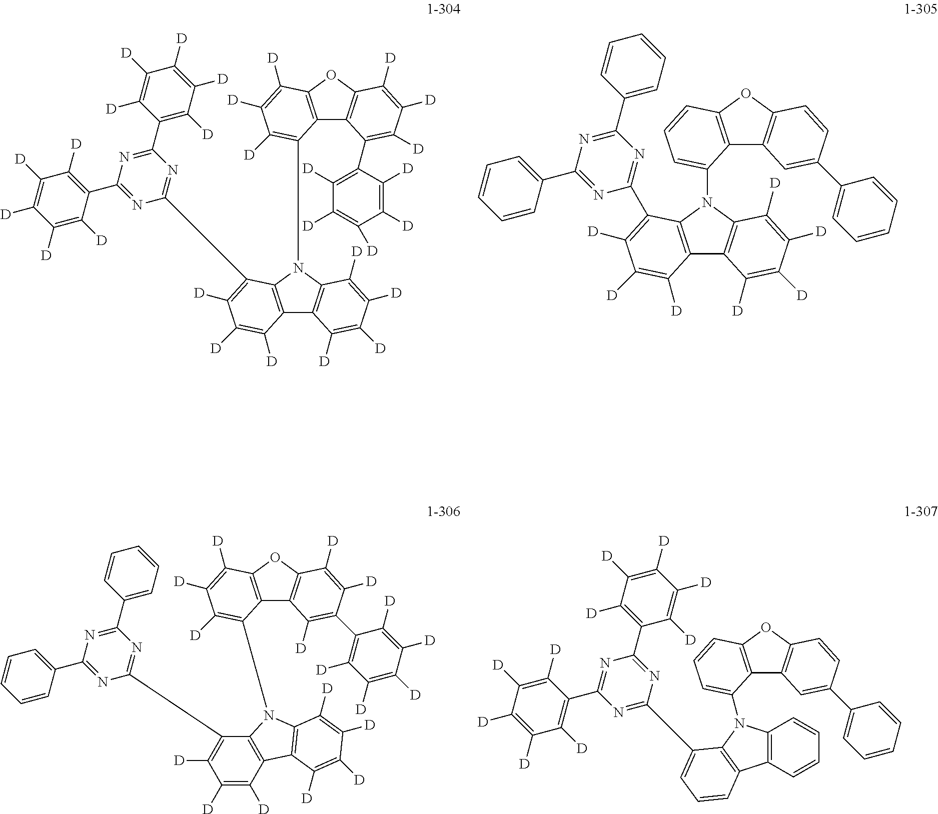

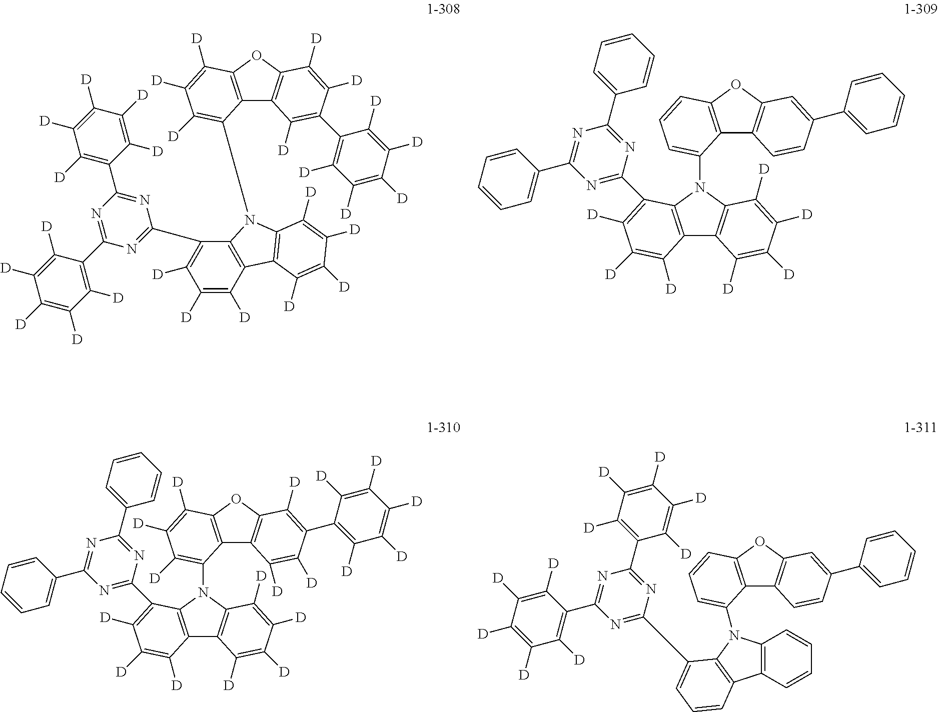

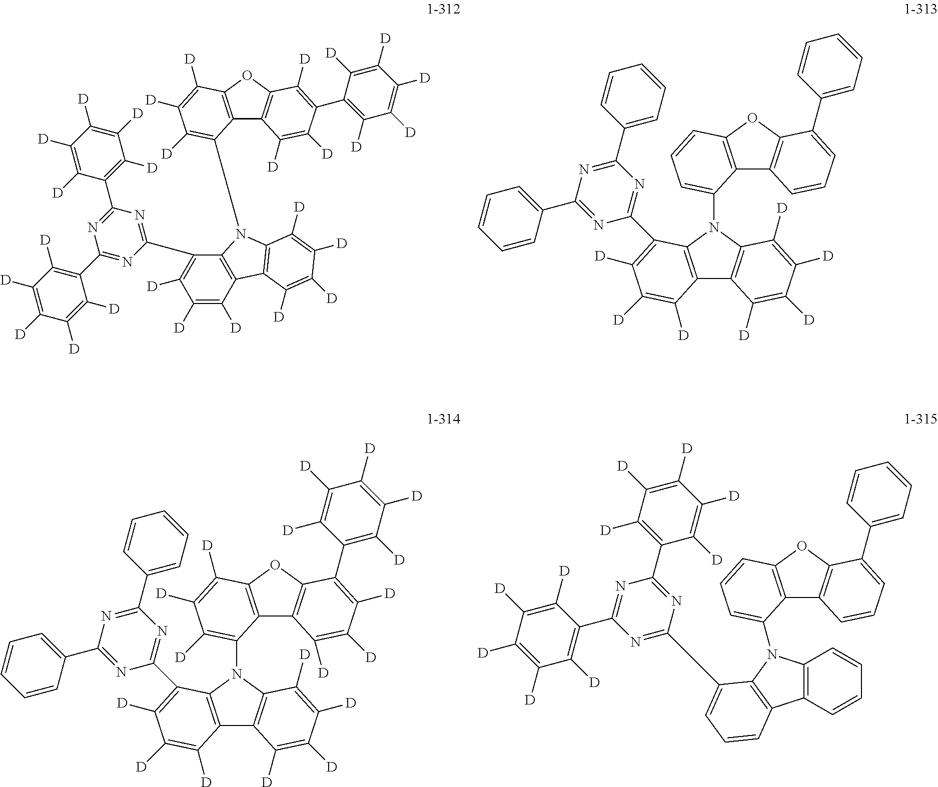

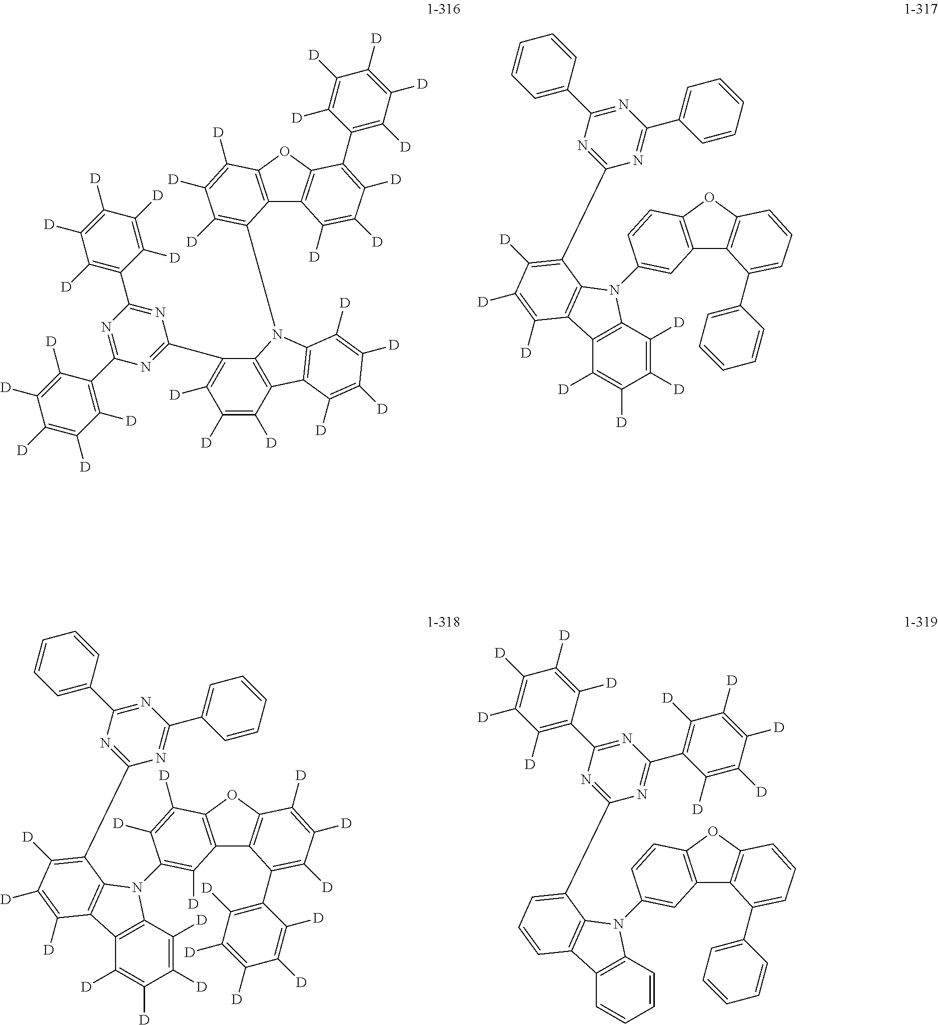

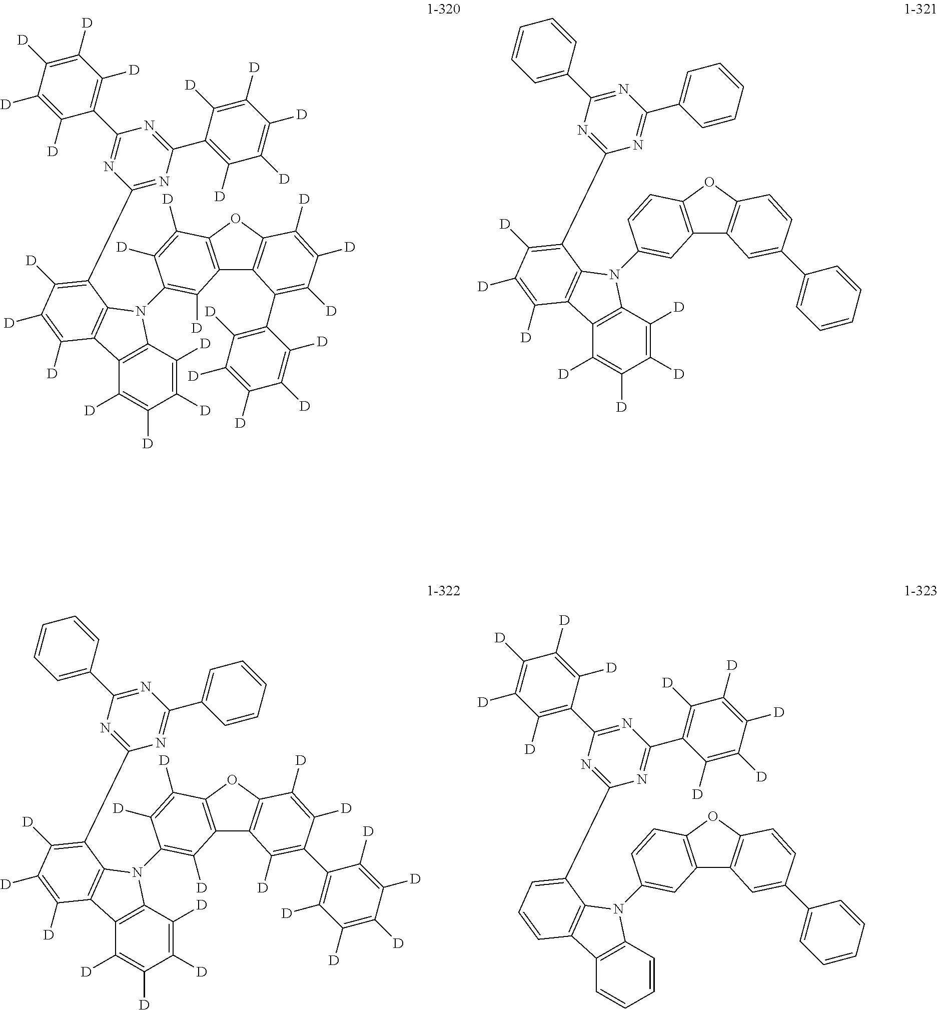

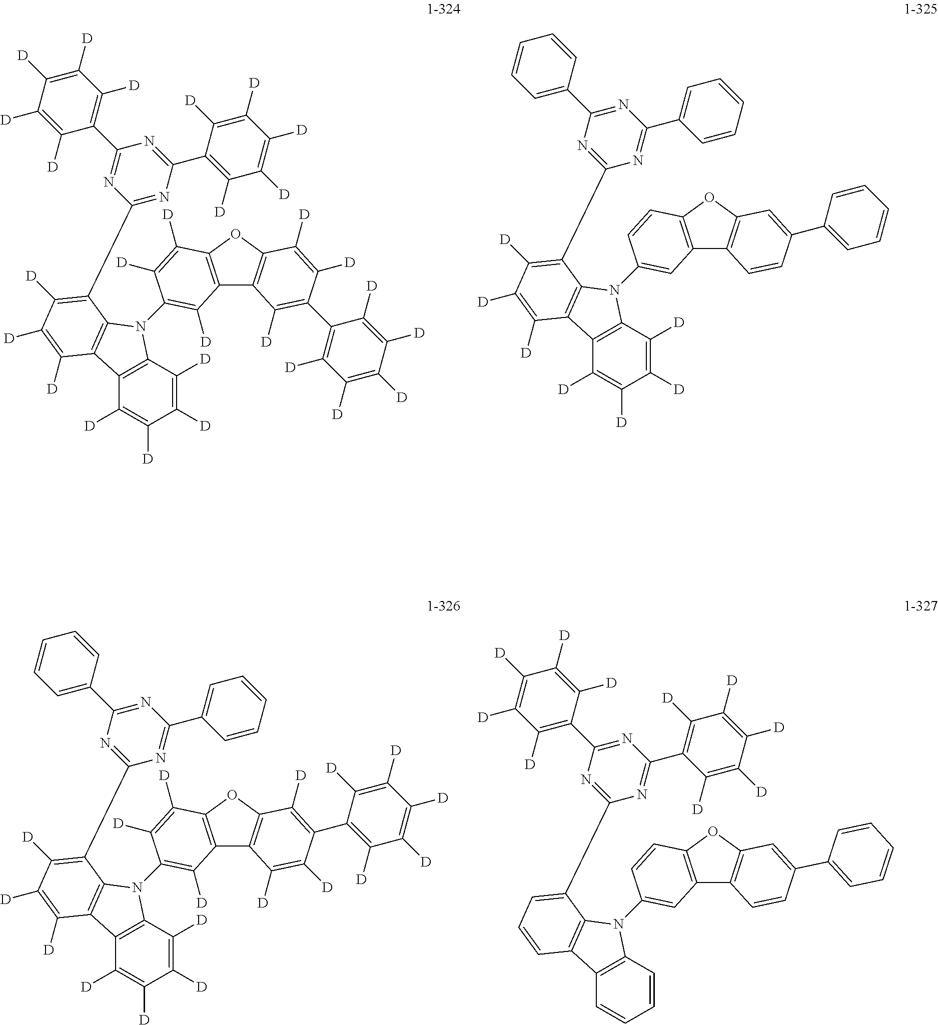

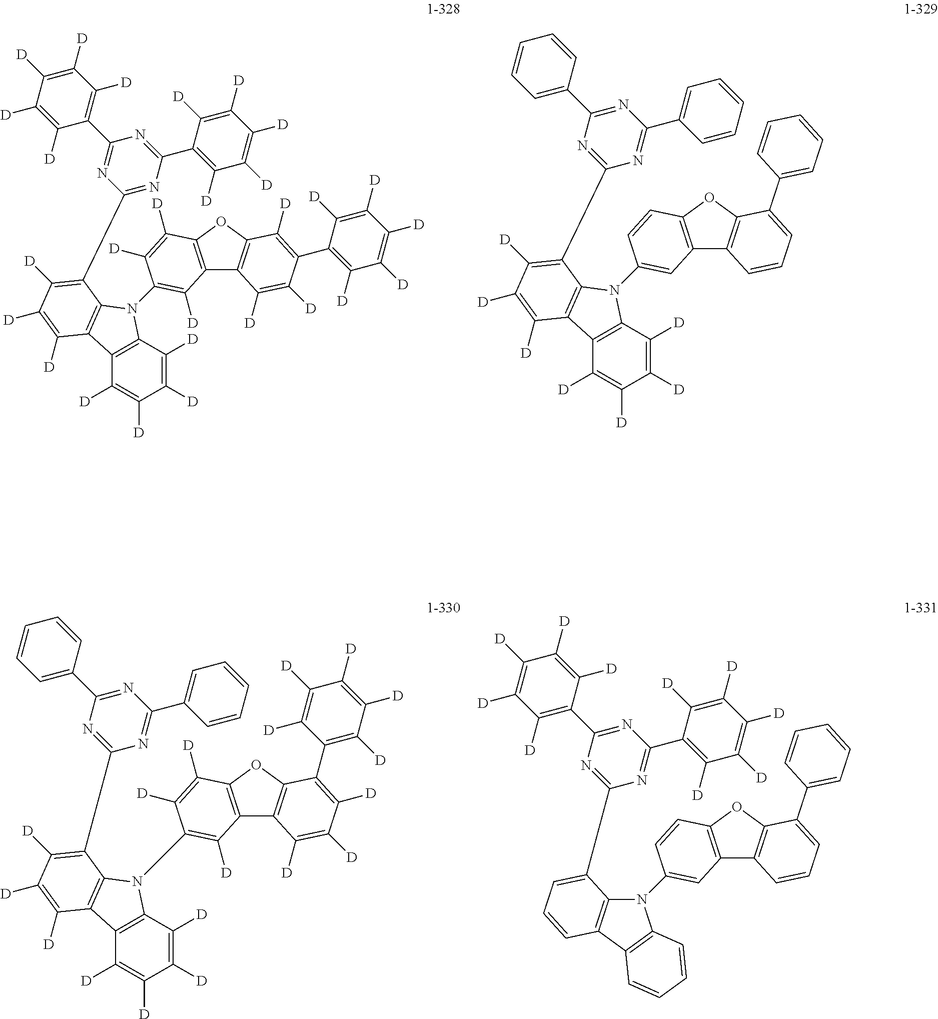

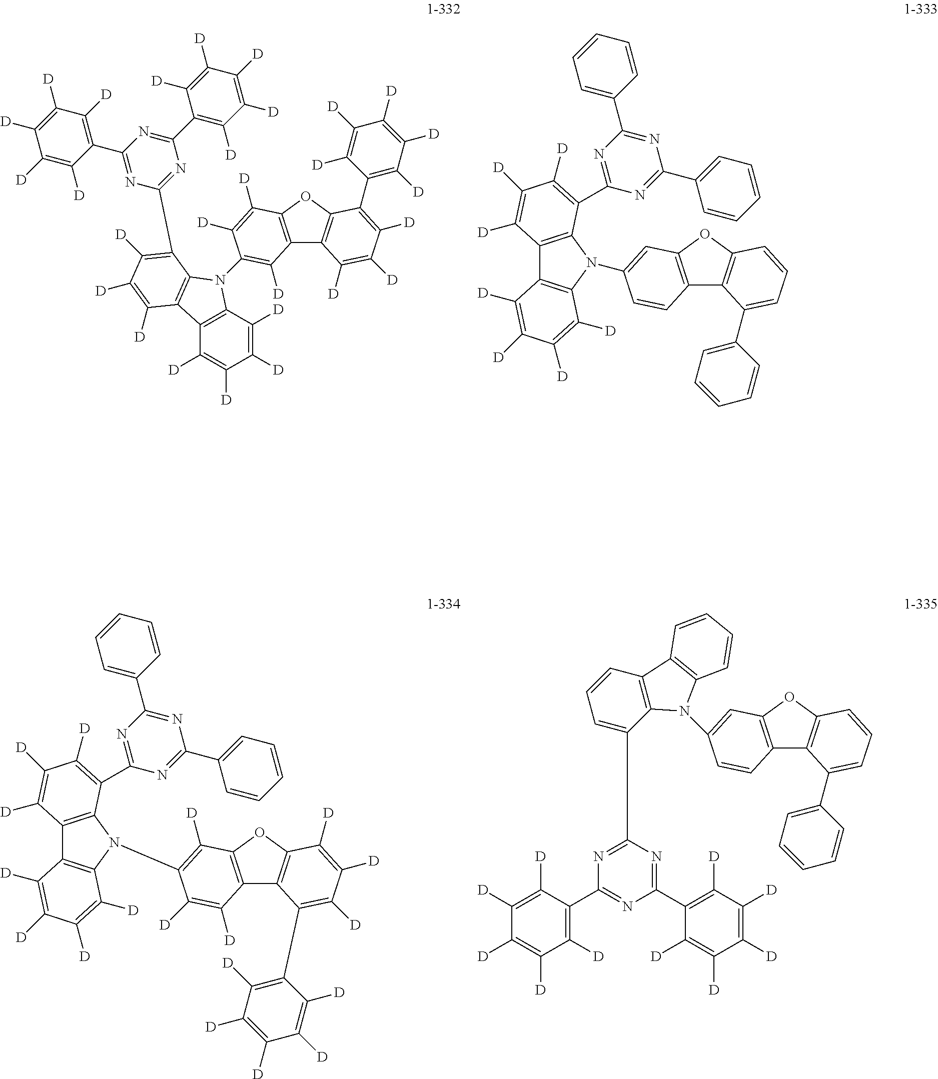

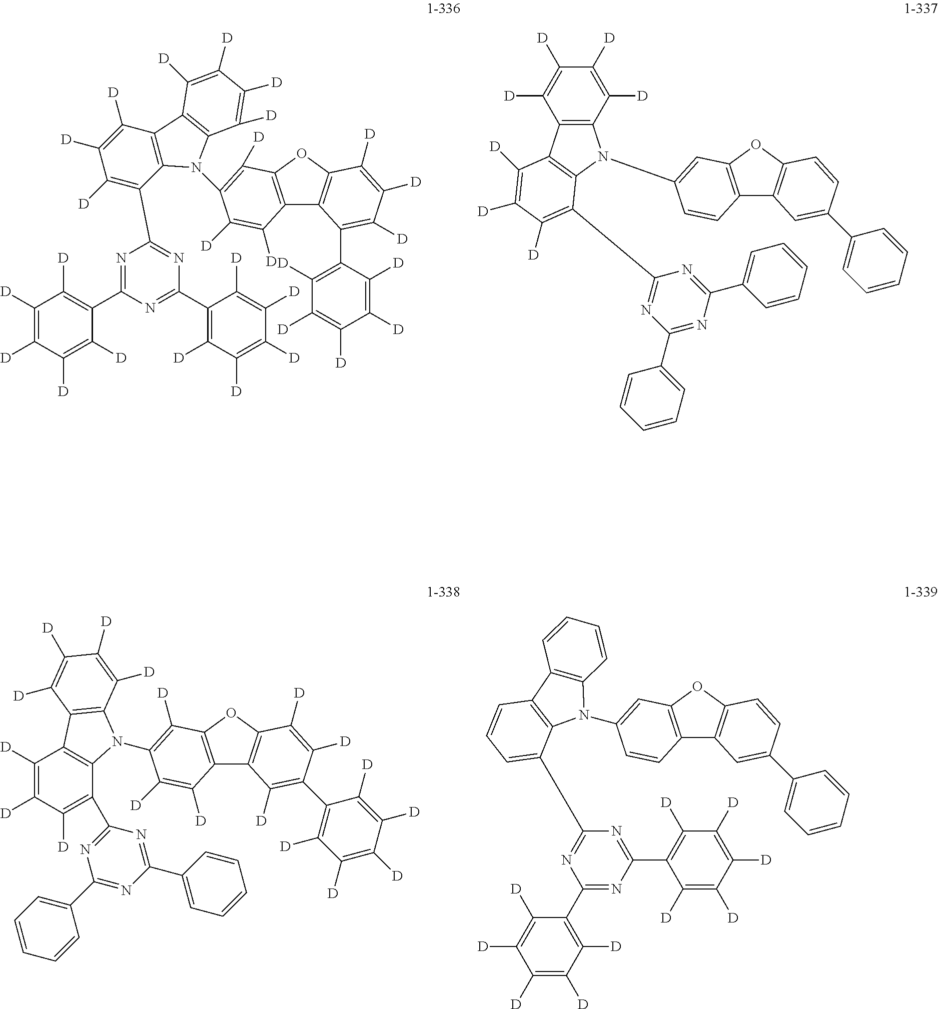

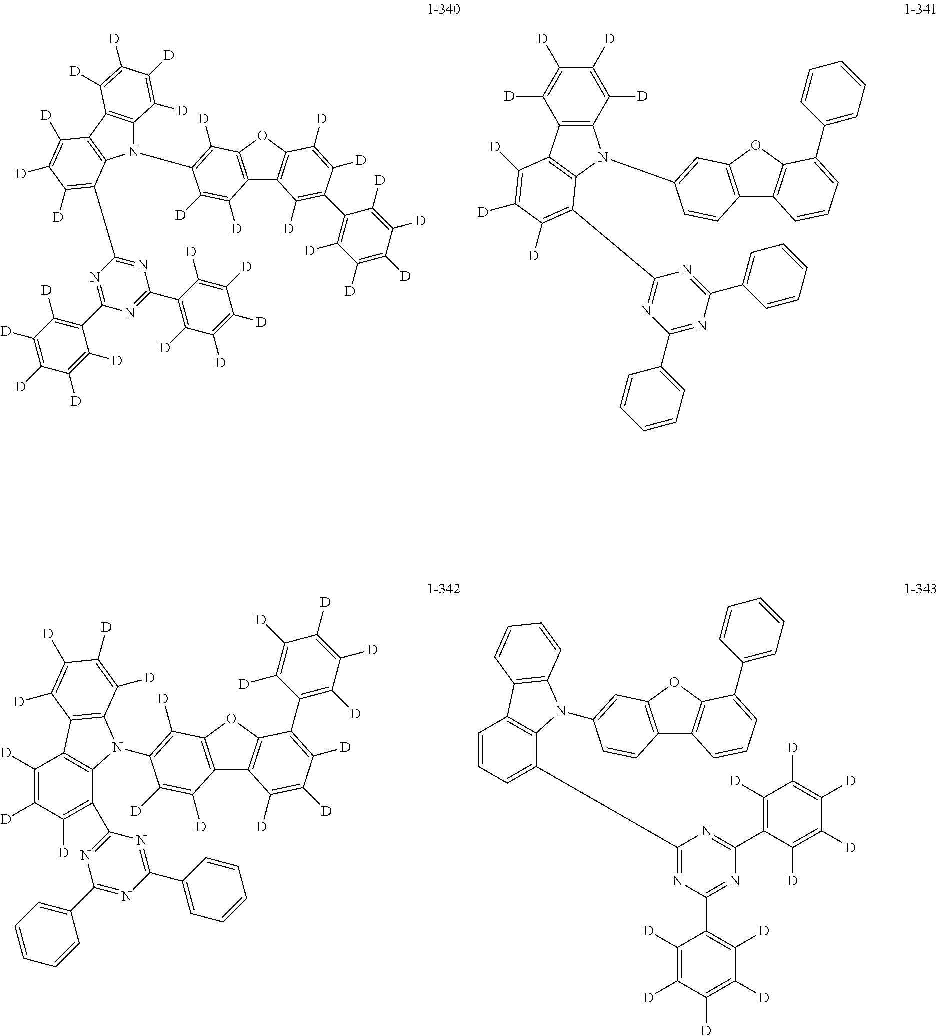

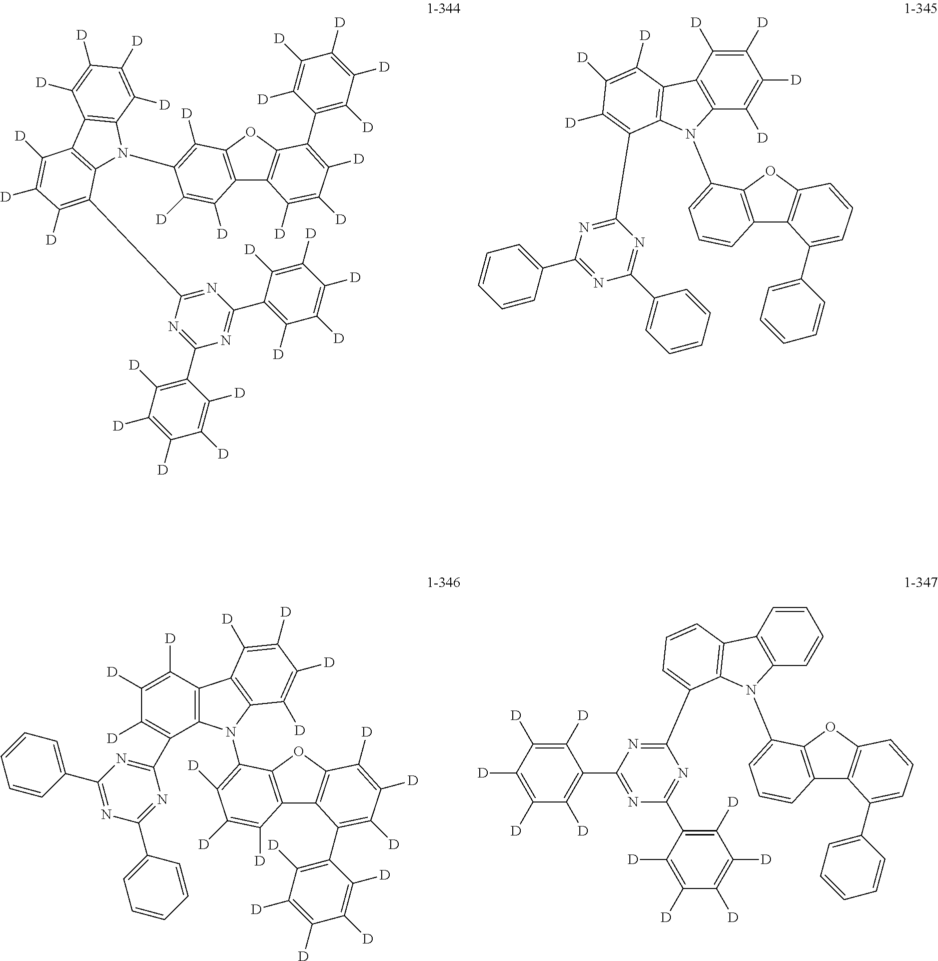

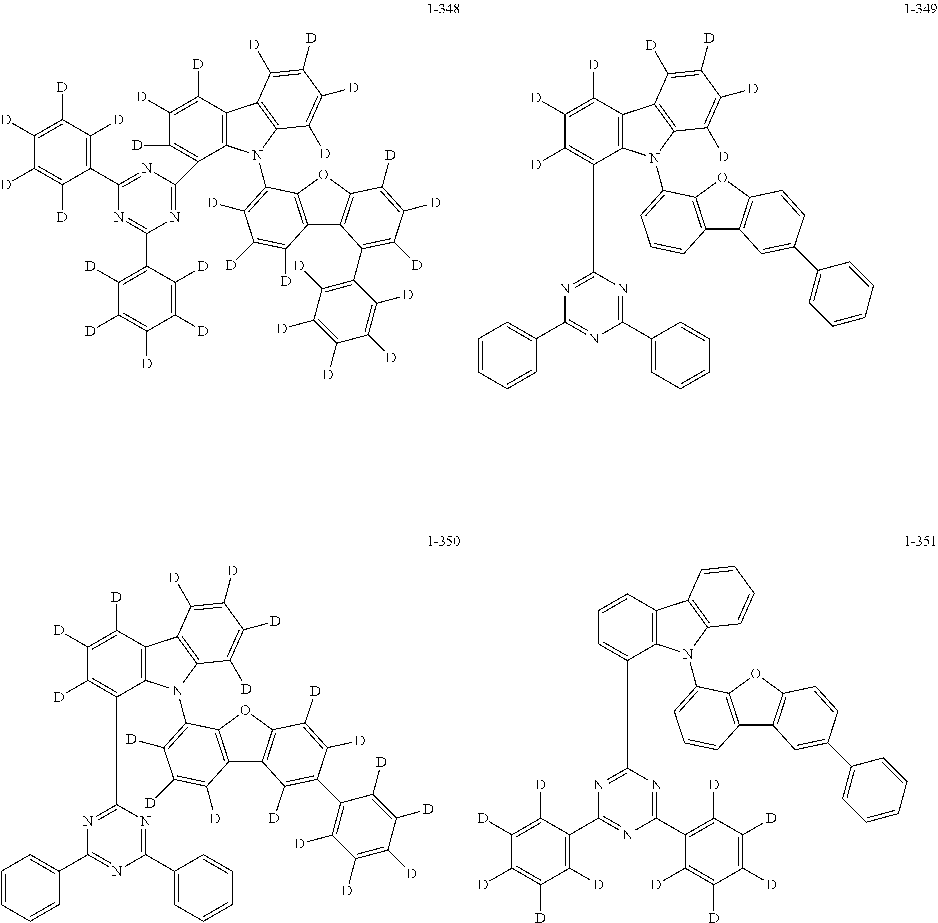

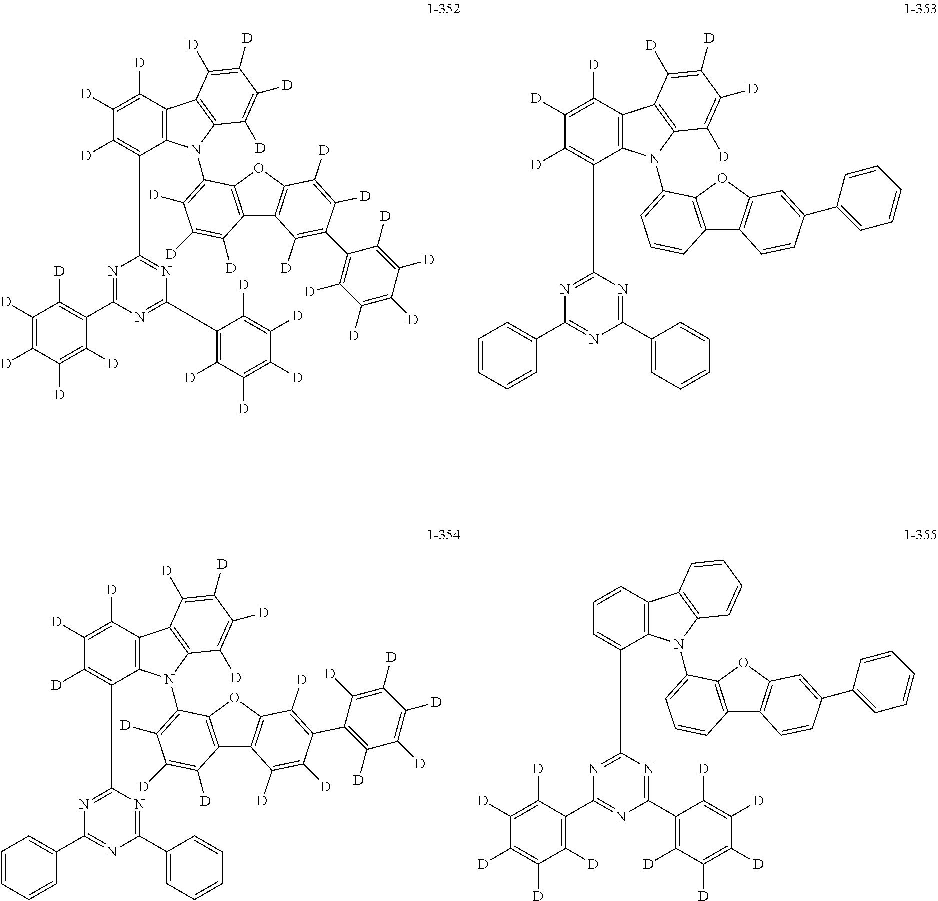

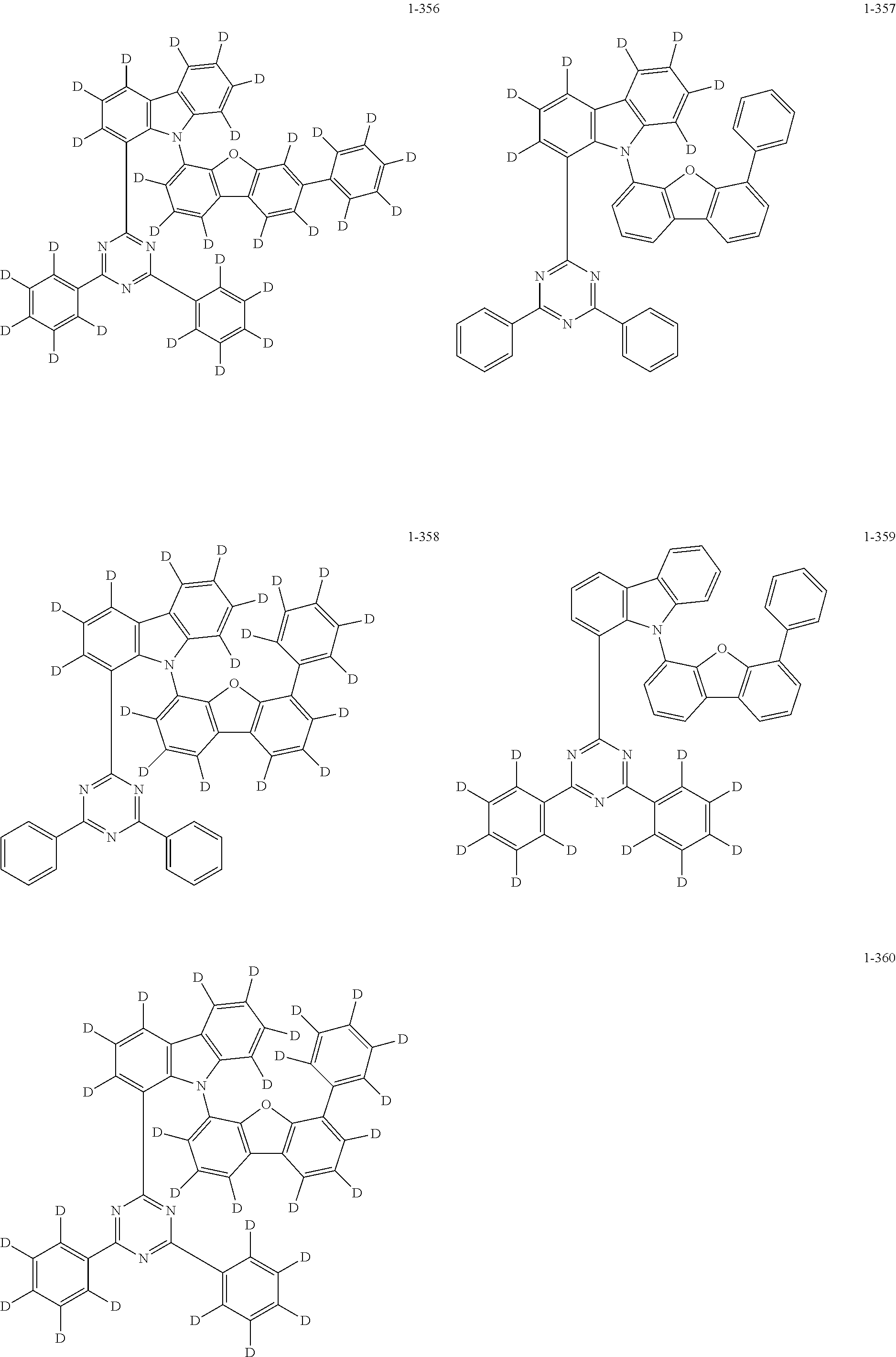

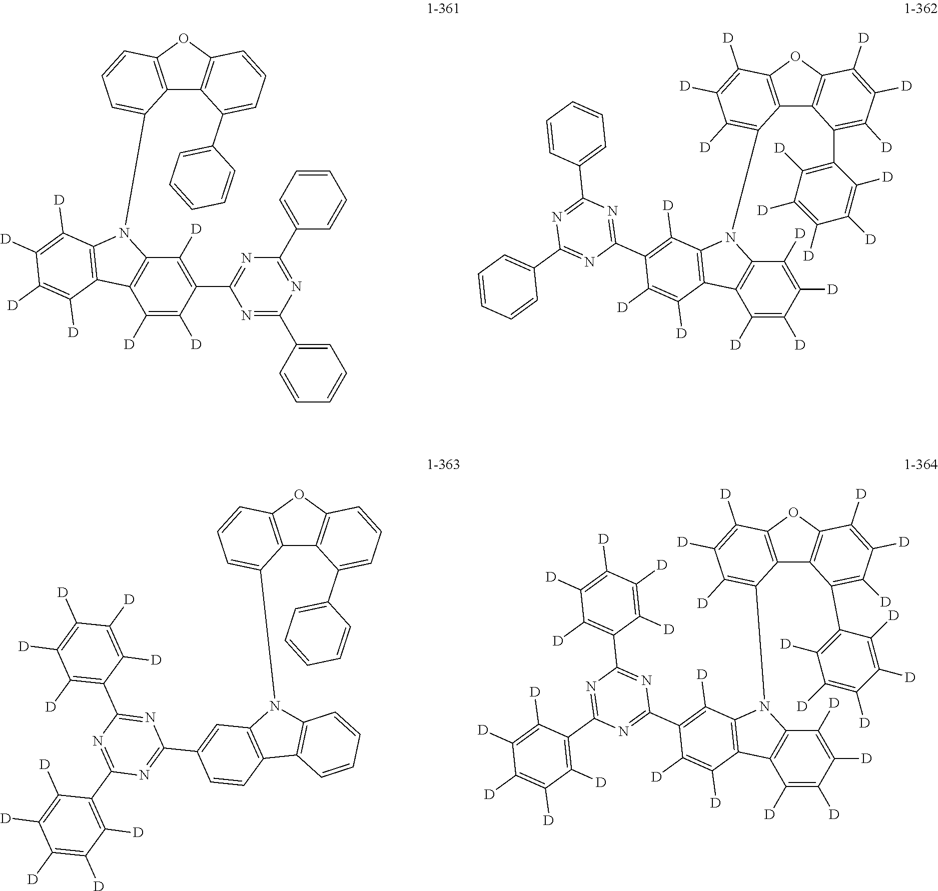

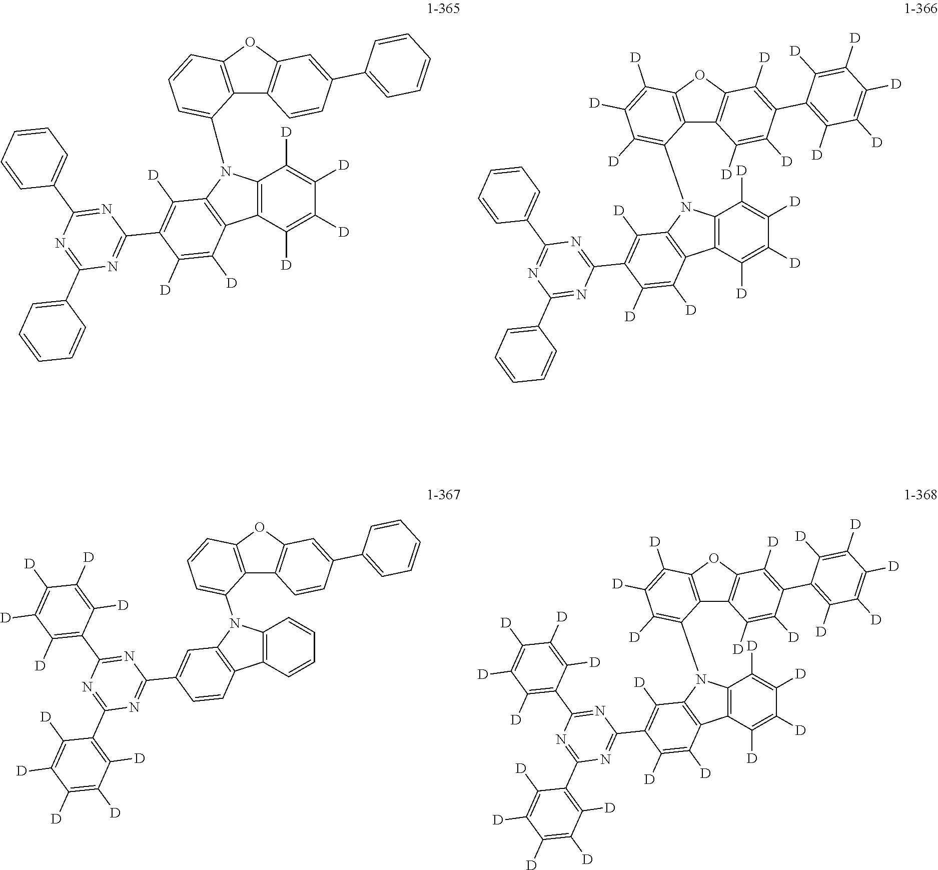

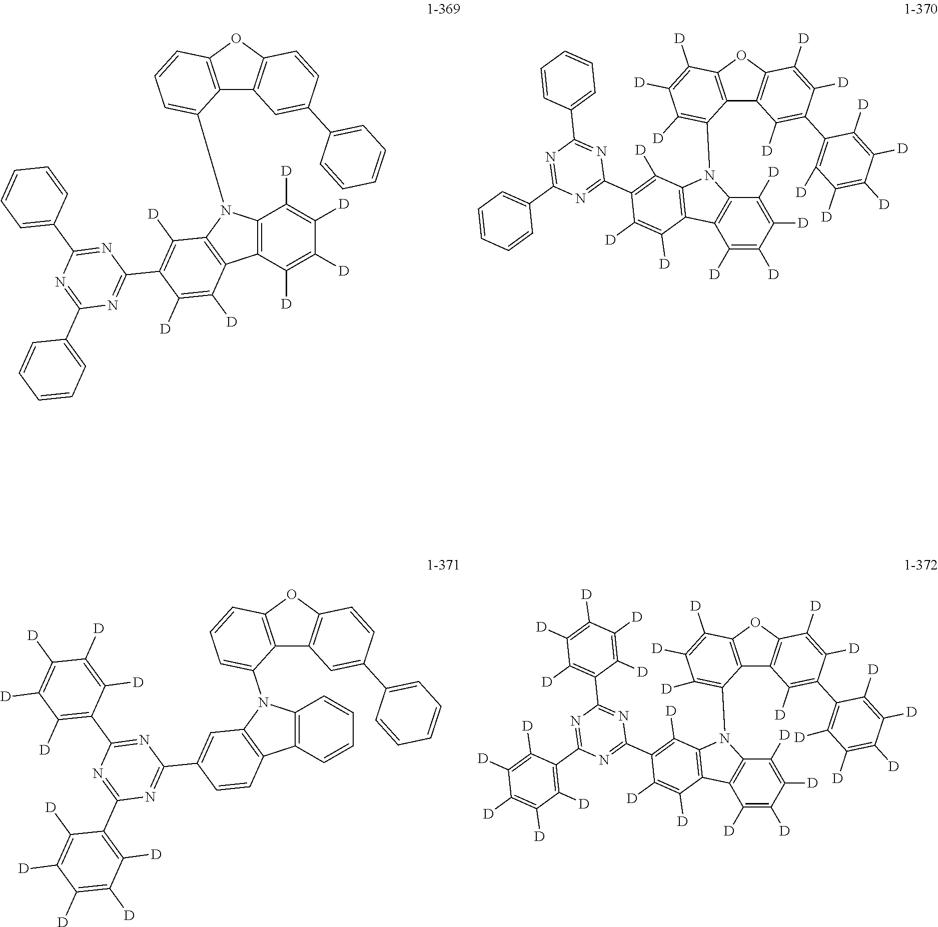

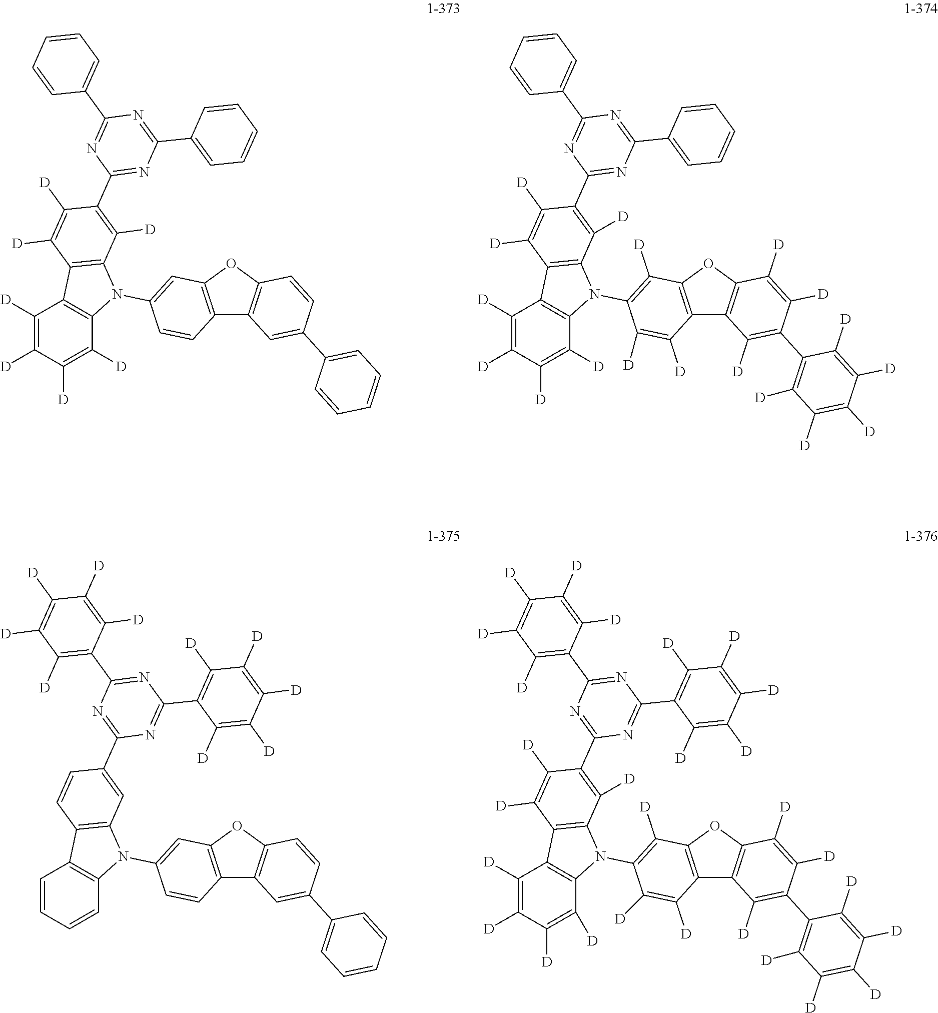

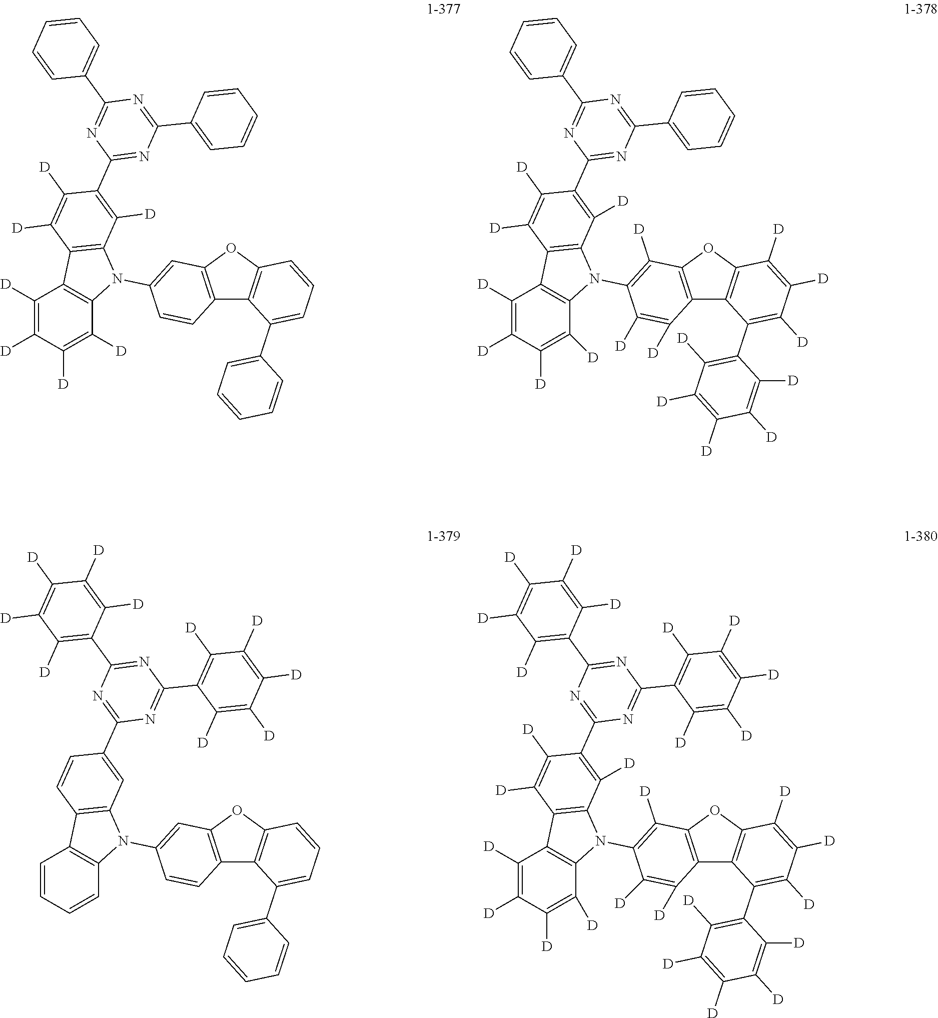

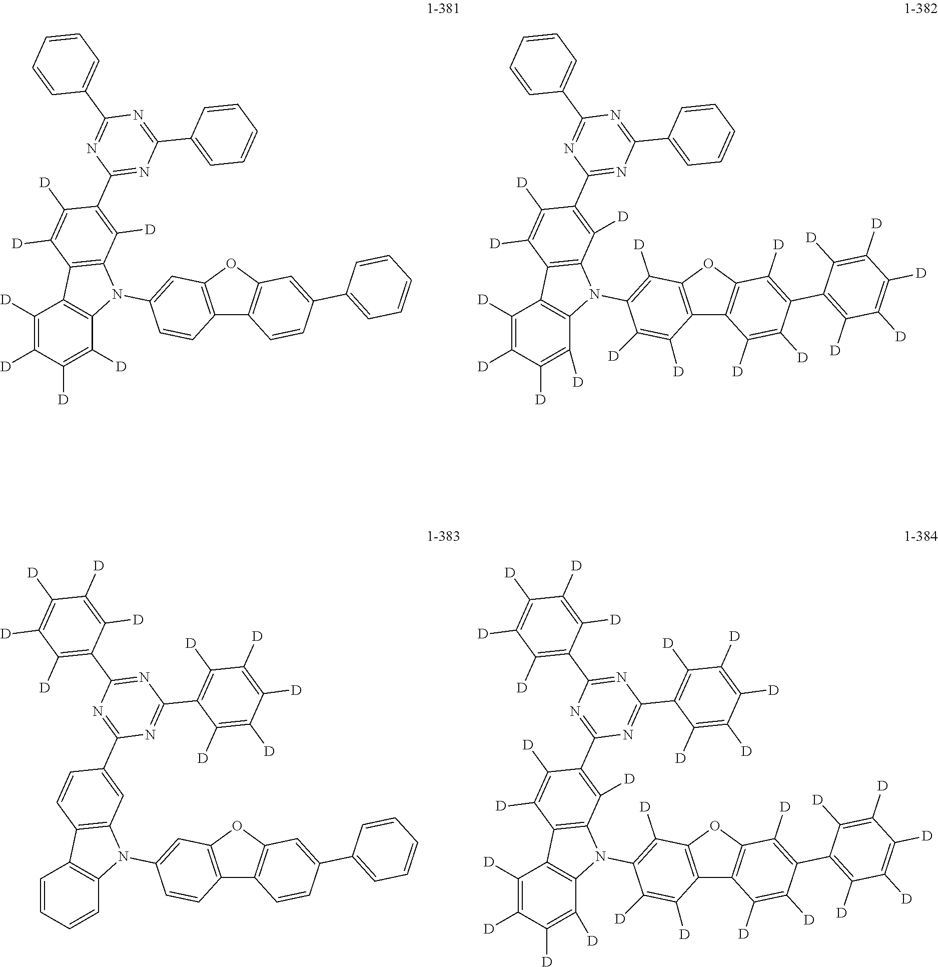

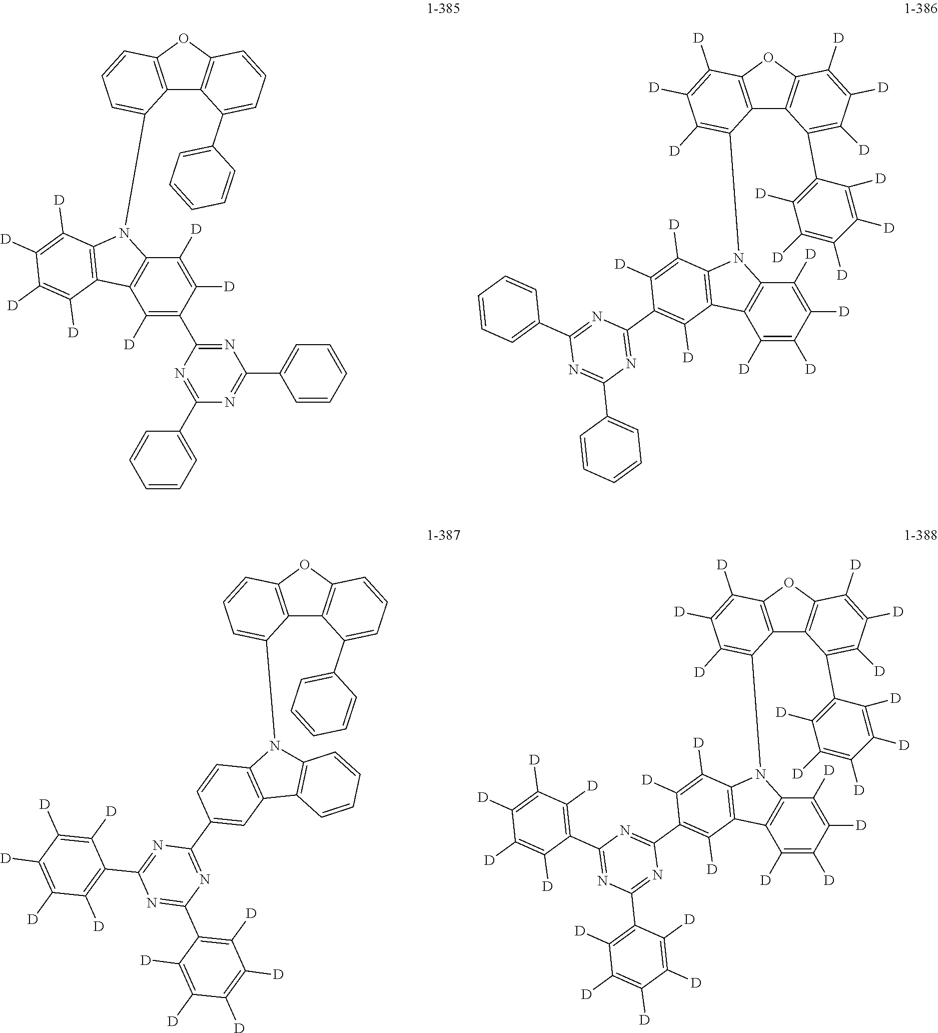

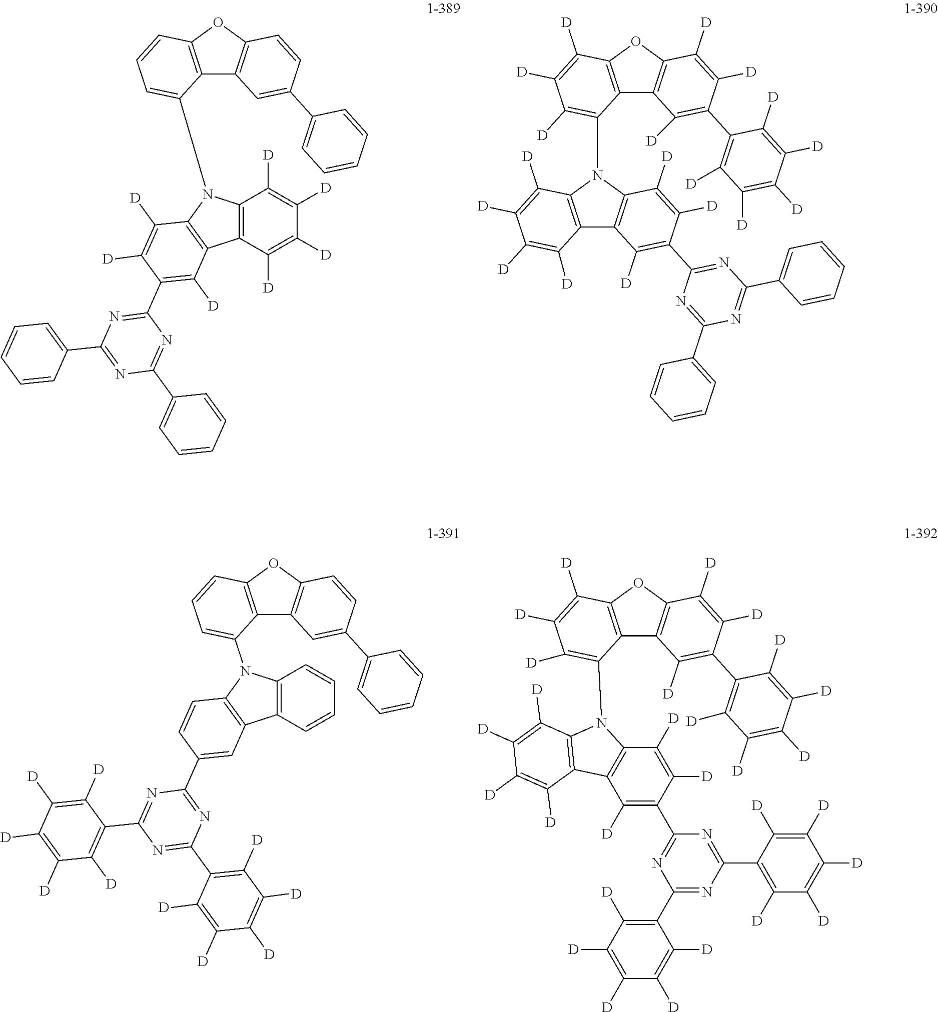

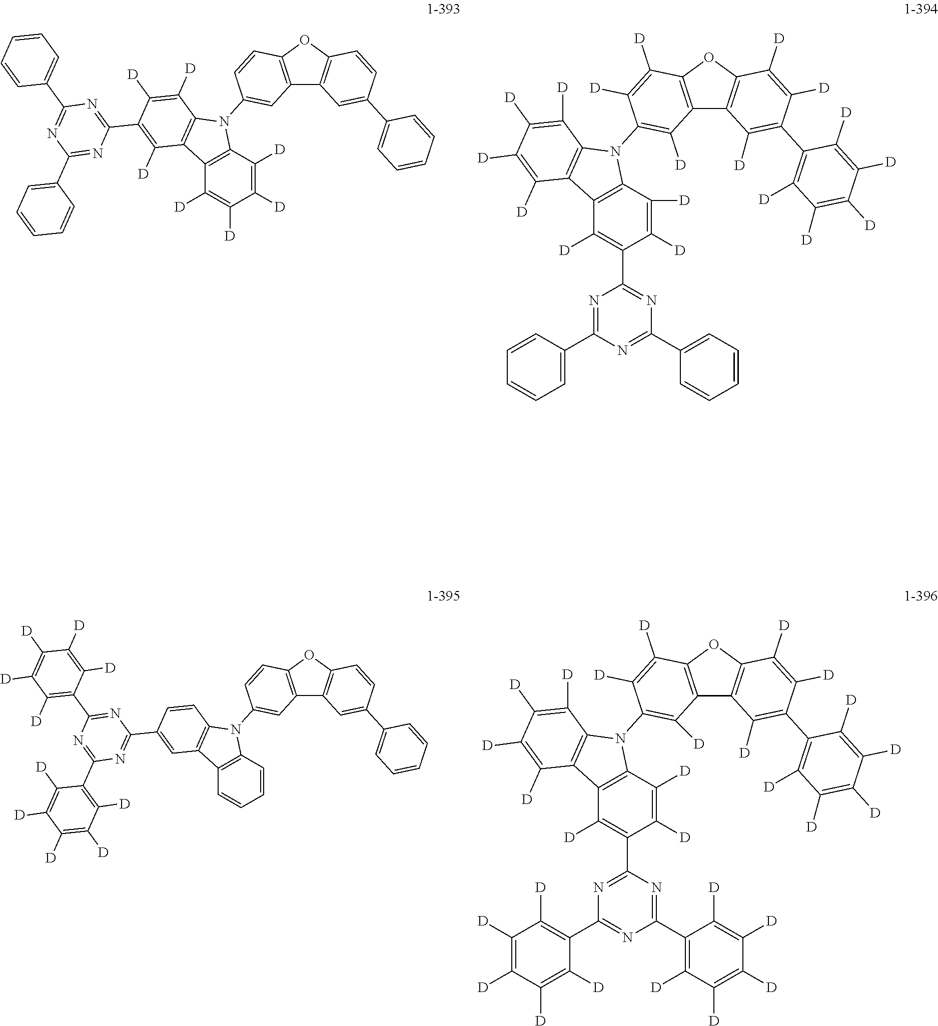

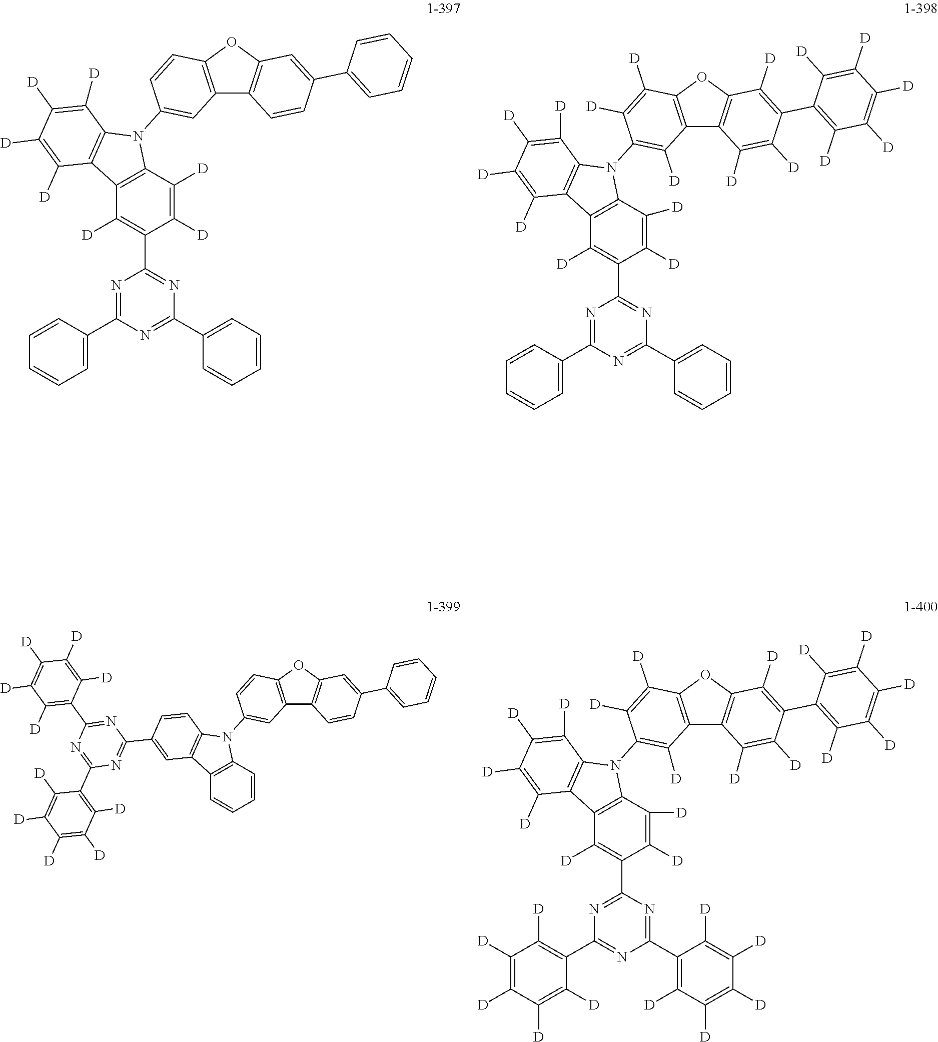

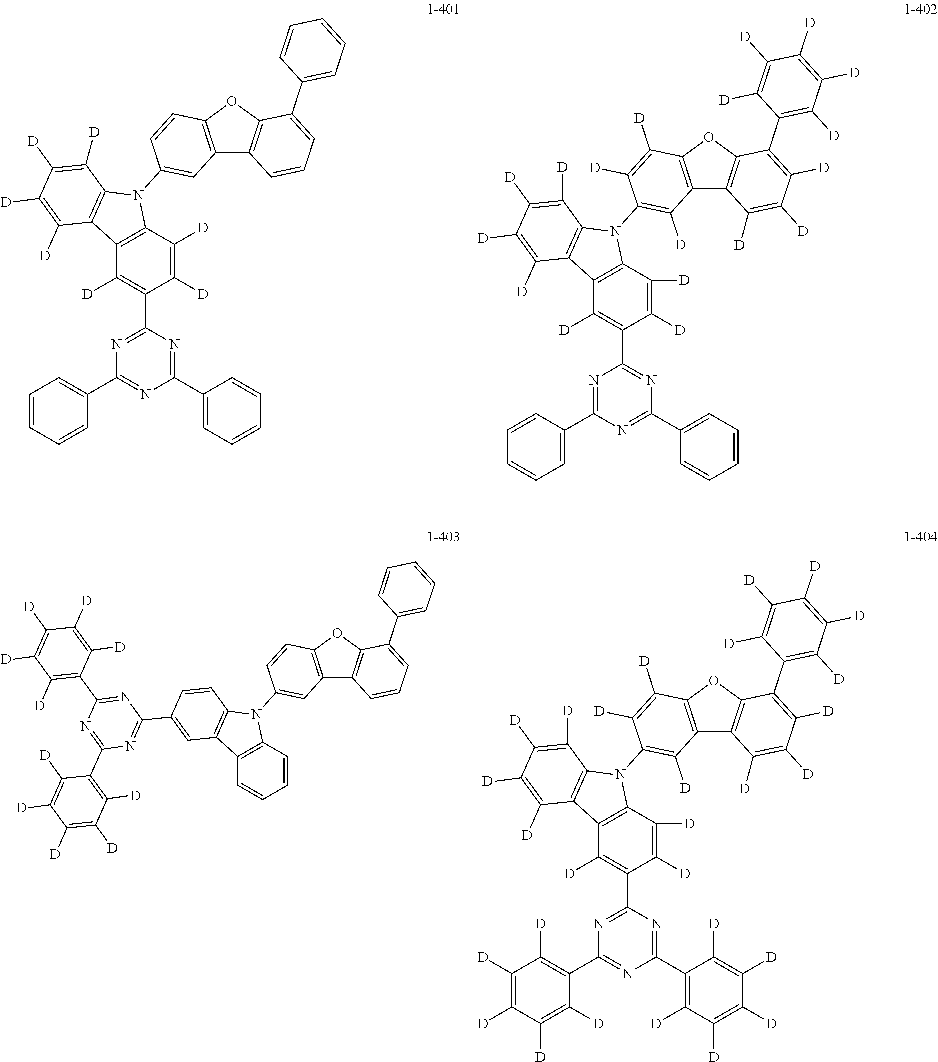

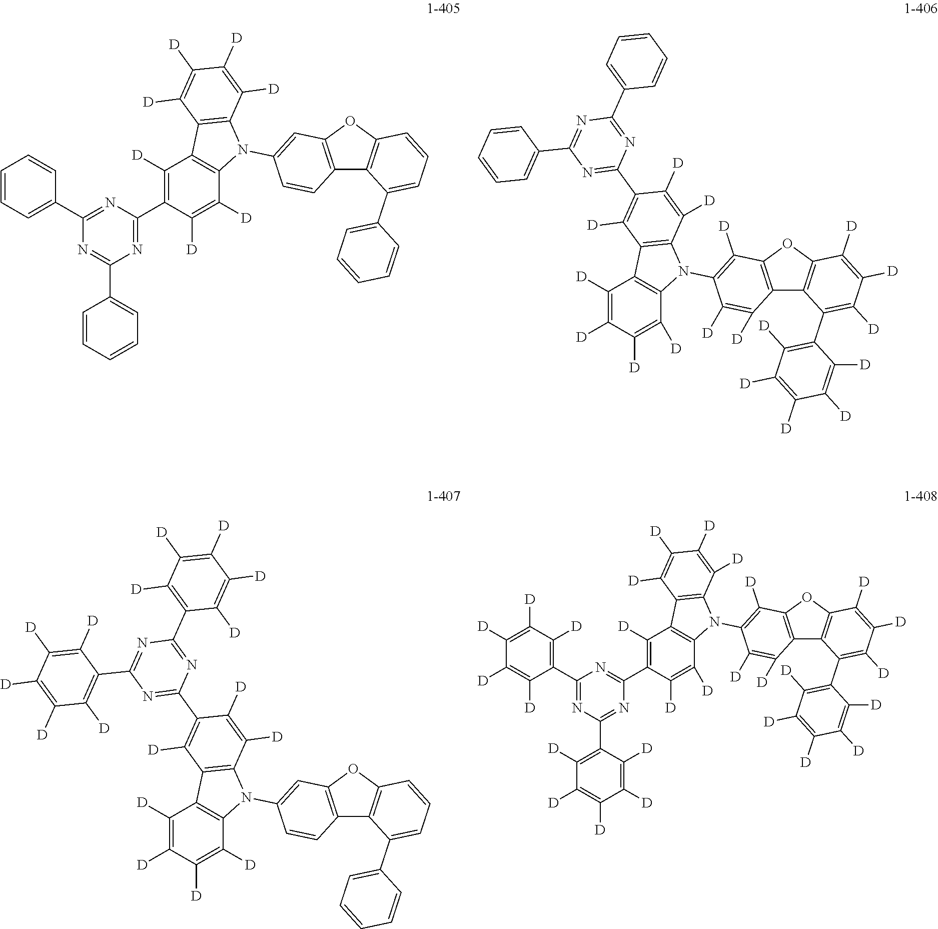

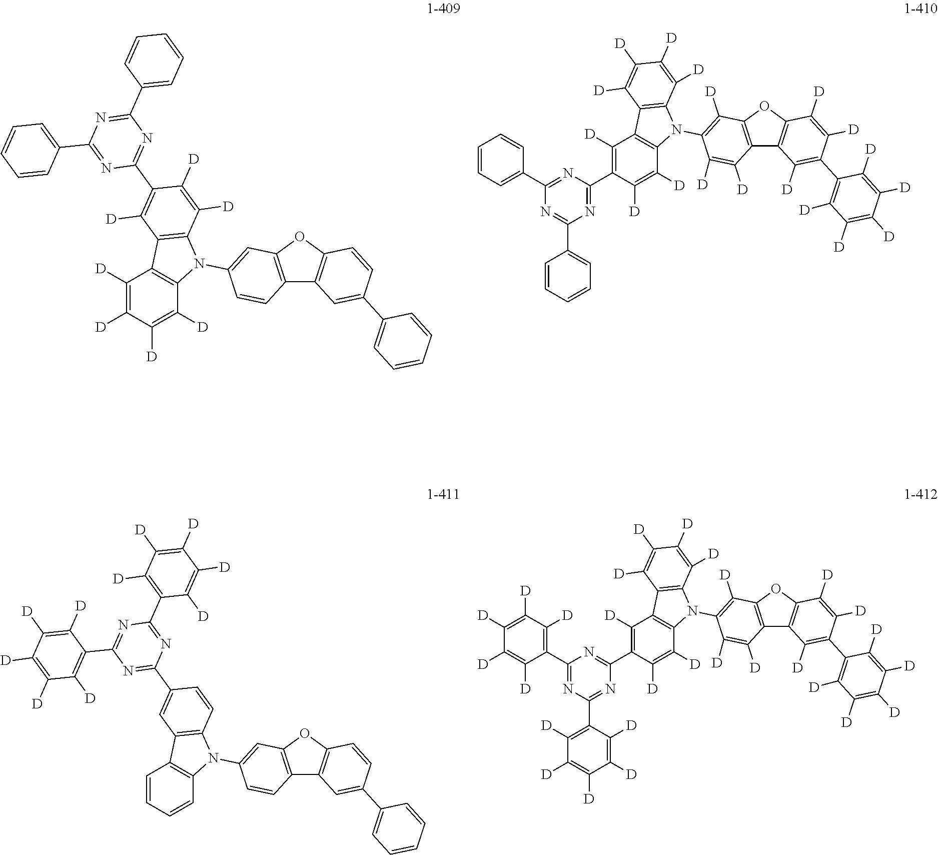

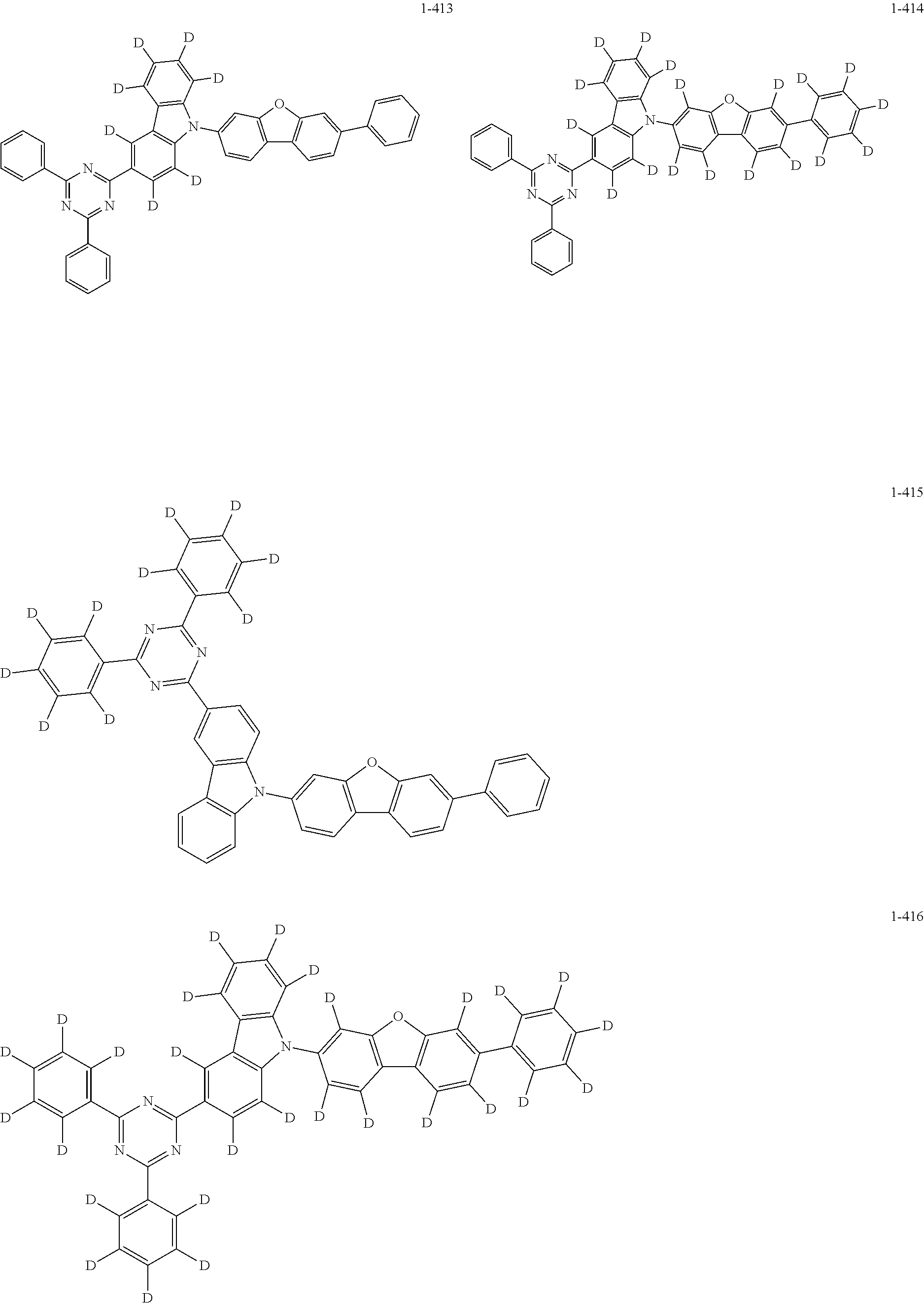

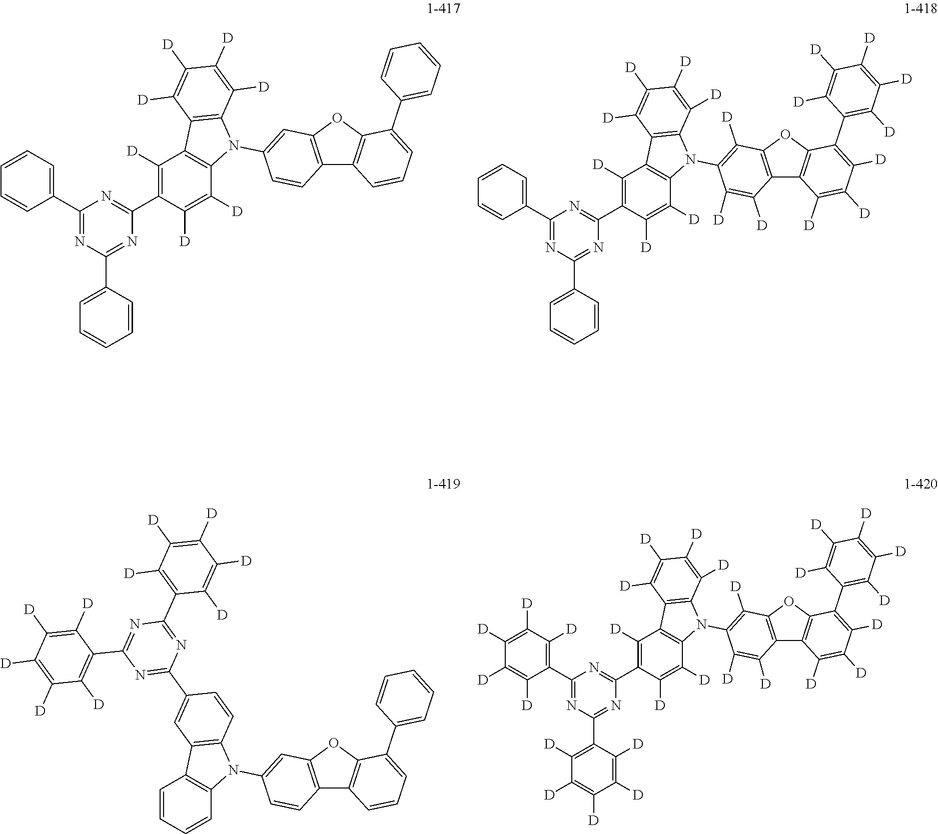

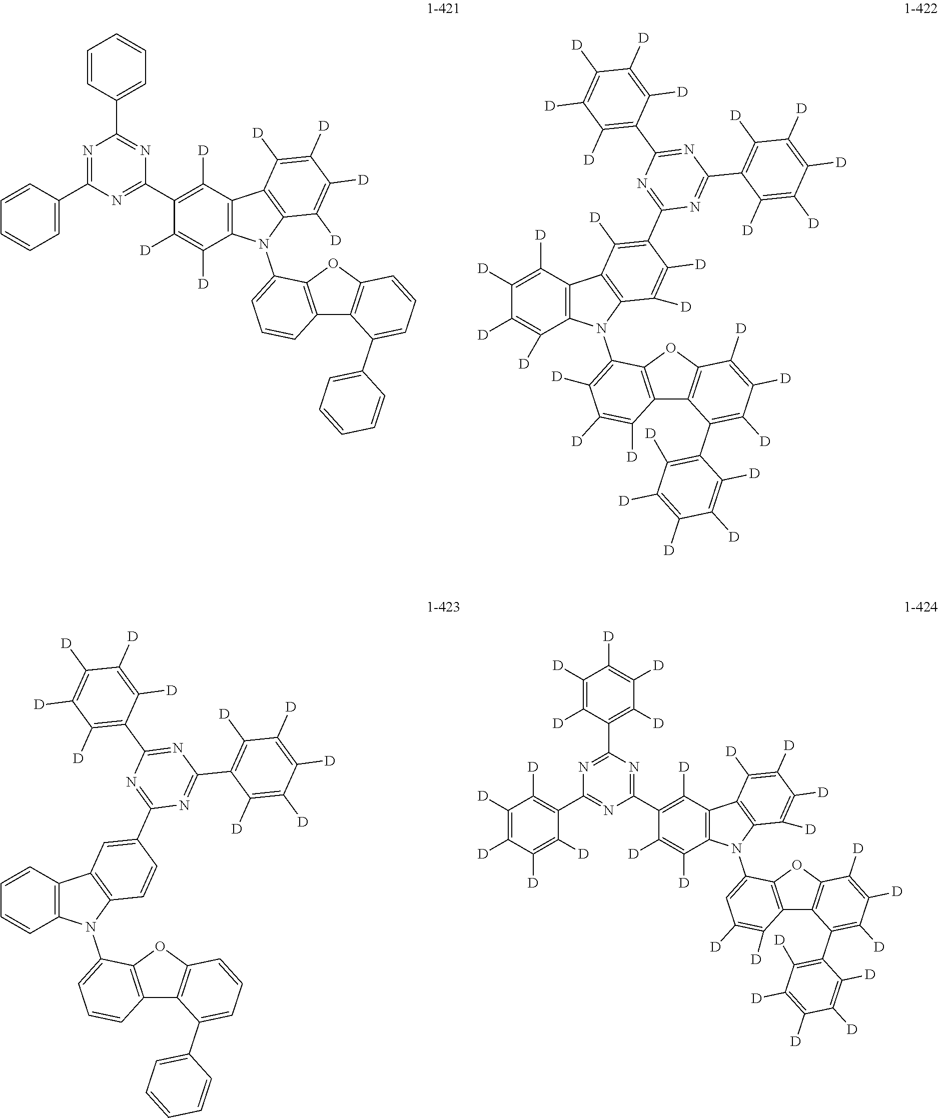

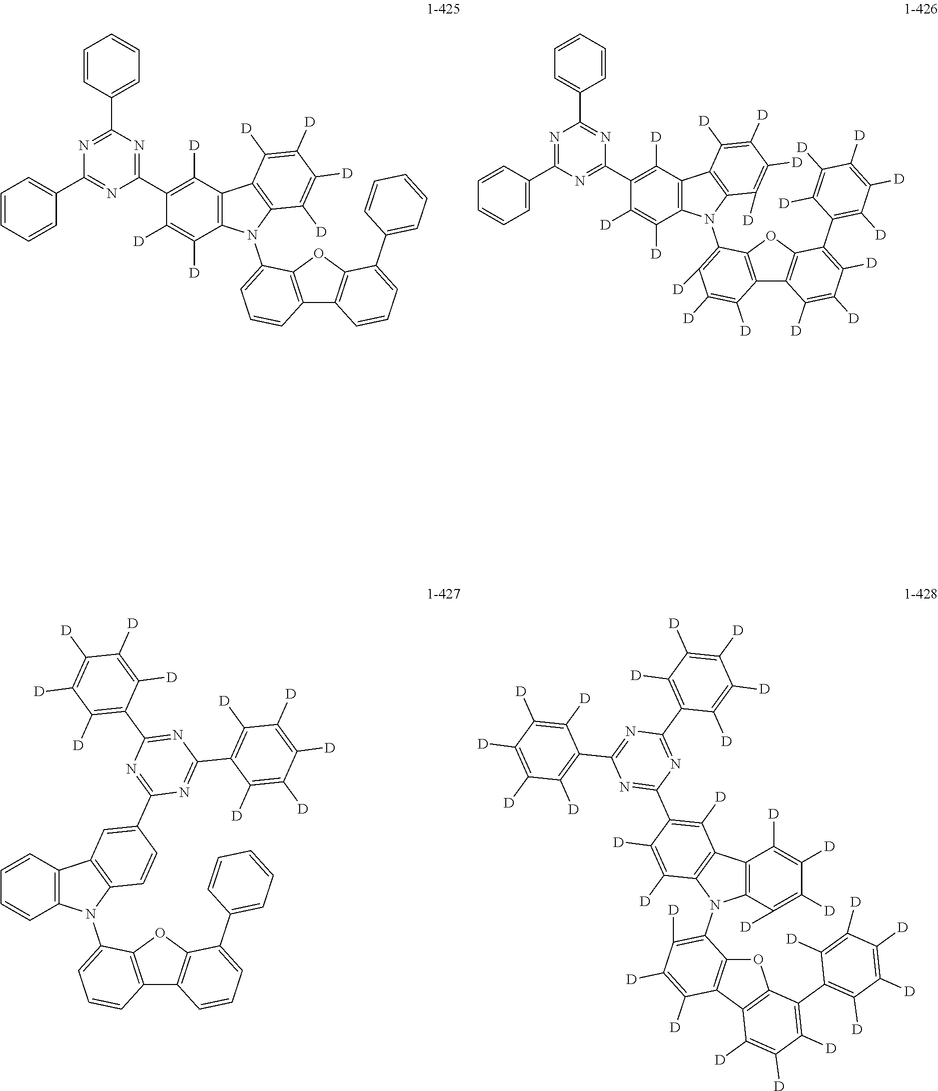

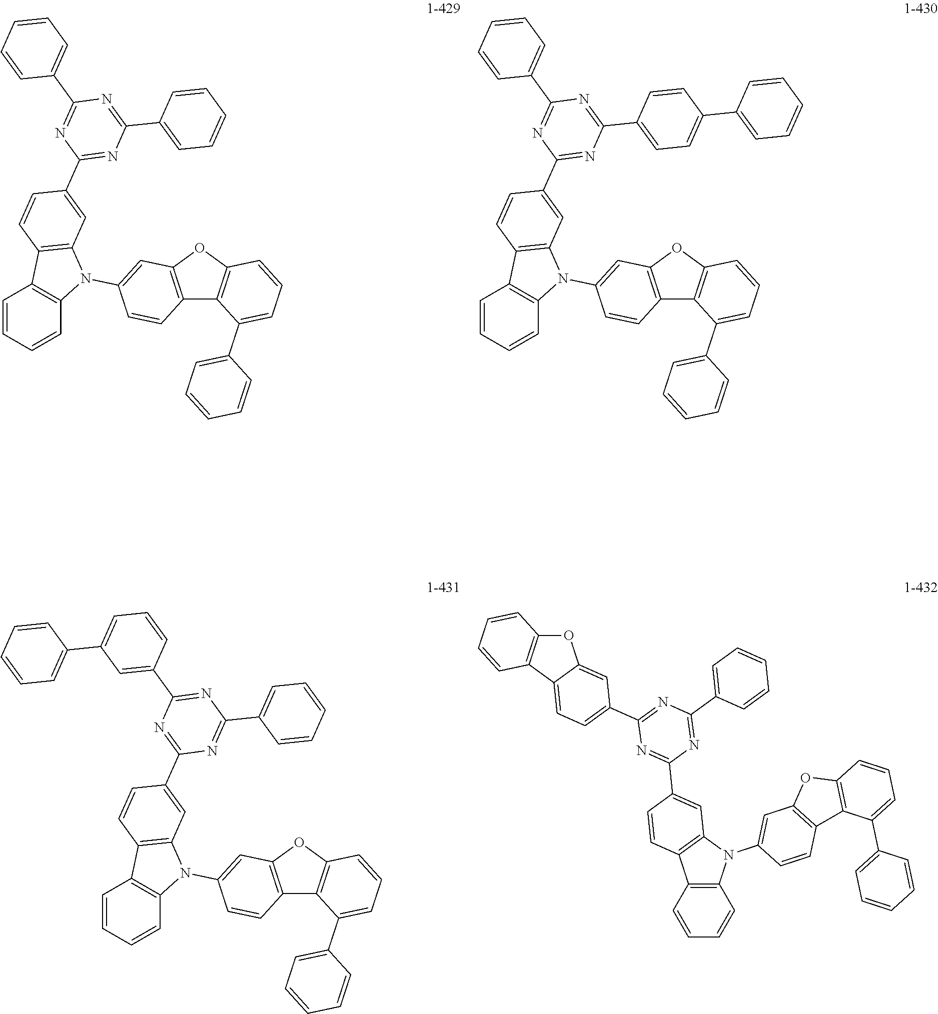 [SMILES: c1ccc(-c2nc(-c3ccccc3)nc(-c3ccc4c5ccccc5n(-c5ccc6c(c5)oc5cccc(-c7ccccc7)c56)c4c3)n2)cc1 ; c1ccc(-c2ccc(-c3nc(-c4ccccc4)nc(-c4ccc5c6ccccc6n(-c6ccc7c(c6)oc6cccc(-c8ccccc8)c67)c5c4)n3)cc2)cc1 ; c1ccc(-c2cccc(-c3nc(-c4ccccc4)nc(-c4ccc5c6ccccc6n(-c6ccc7c(c6)oc6cccc(-c8ccccc8)c67)c5c4)n3)c2)cc1 ; c1ccc(-c2nc(-c3ccc4c(c3)oc3ccccc34)nc(-c3ccc4c5ccccc5n(-c5ccc6c(c5)oc5cccc(-c7ccccc7)c56)c4c3)n2)cc1]

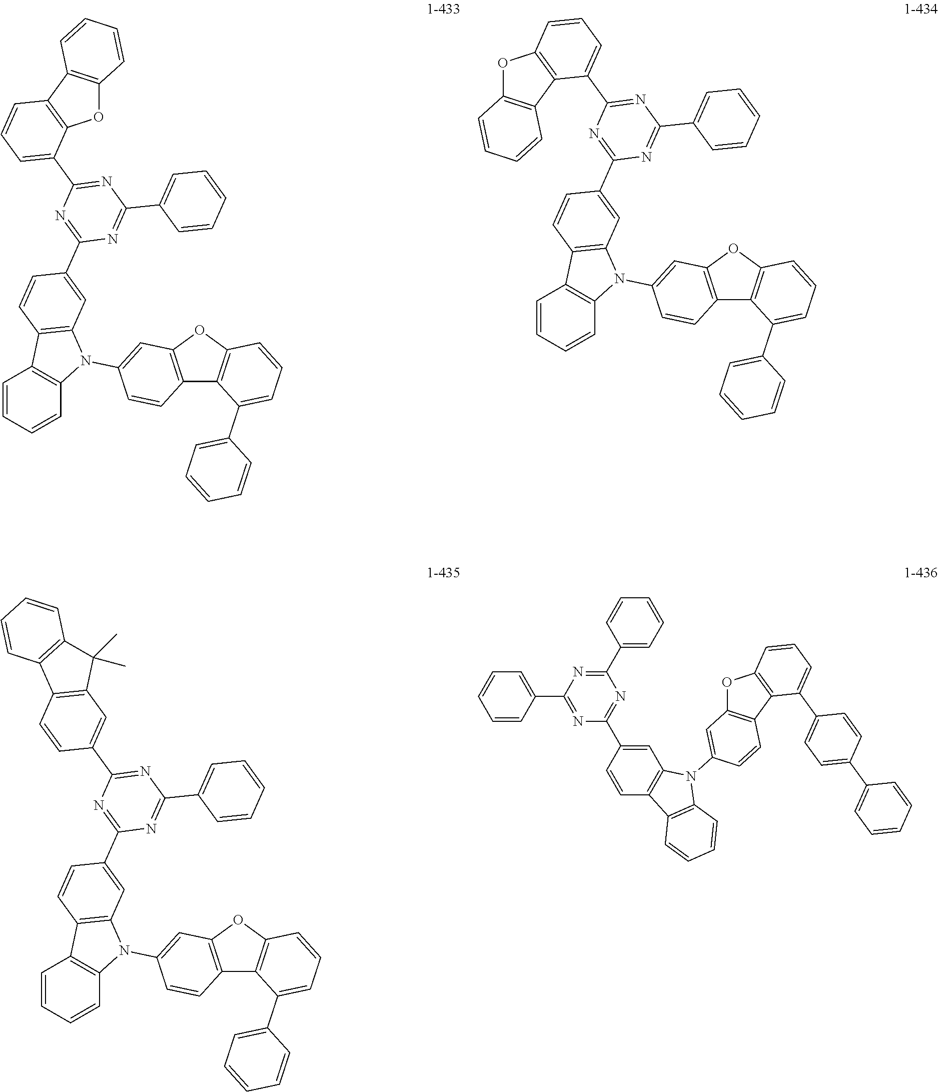 [SMILES: c1ccc(-c2nc(-c3ccc4c5ccccc5n(-c5ccc6c(c5)oc5cccc(-c7ccccc7)c56)c4c3)nc(-c3cccc4c3oc3ccccc34)n2)cc1 ; c1ccc(-c2nc(-c3ccc4c5ccccc5n(-c5ccc6c(c5)oc5cccc(-c7ccccc7)c56)c4c3)nc(-c3cccc4oc5ccccc5c34)n2)cc1 ; CC1(C)c2ccccc2-c2ccc(-c3nc(-c4ccccc4)nc(-c4ccc5c6ccccc6n(-c6ccc7c(c6)oc6cccc(-c8ccccc8)c67)c5c4)n3)cc21 ; c1ccc(-c2ccc(-c3cccc4oc5cc(-n6c7ccccc7c7ccc(-c8nc(-c9ccccc9)nc(-c9ccccc9)n8)cc76)ccc5c34)cc2)cc1]

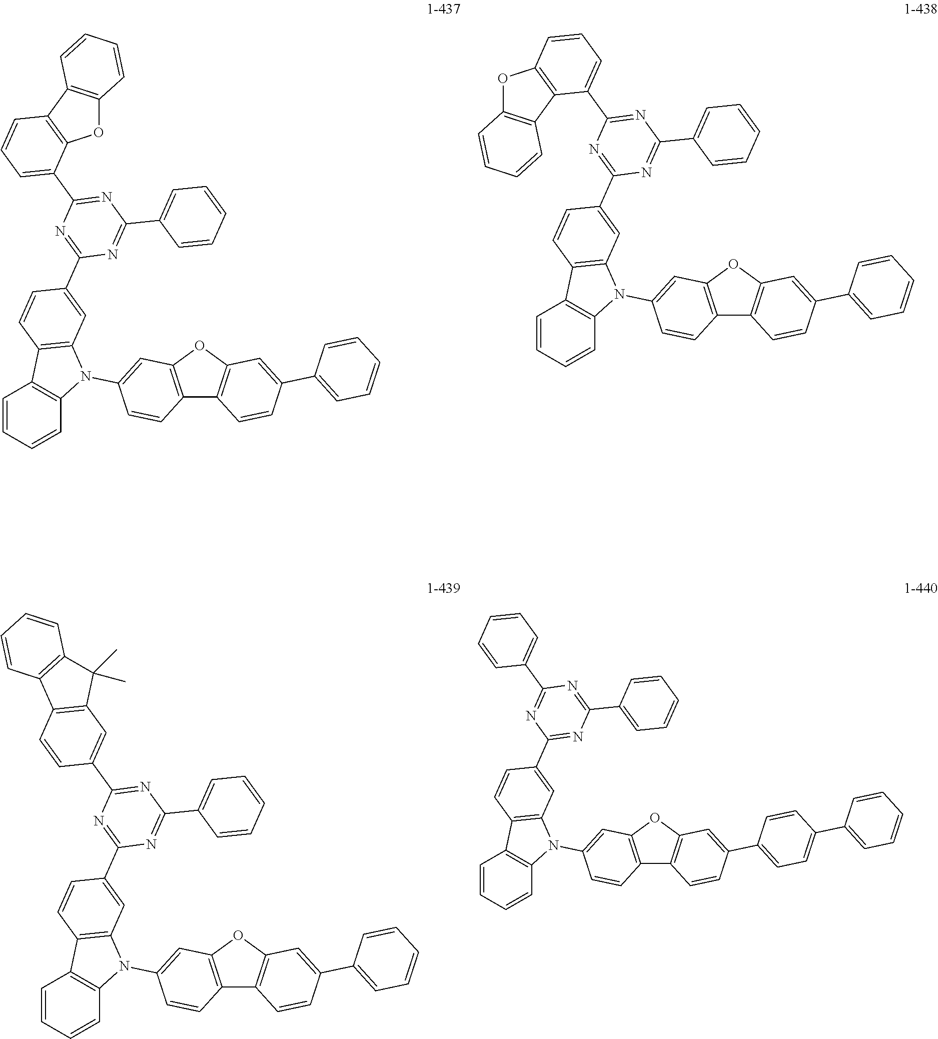 [SMILES: c1ccc(-c2ccc3c(c2)oc2cc(-n4c5ccccc5c5ccc(-c6nc(-c7ccccc7)nc(-c7cccc8c7oc7ccccc78)n6)cc54)ccc23)cc1 ; c1ccc(-c2ccc3c(c2)oc2cc(-n4c5ccccc5c5ccc(-c6nc(-c7ccccc7)nc(-c7cccc8oc9ccccc9c78)n6)cc54)ccc23)cc1 ; CC1(C)c2ccccc2-c2ccc(-c3nc(-c4ccccc4)nc(-c4ccc5c6ccccc6n(-c6ccc7c(c6)oc6cc(-c8ccccc8)ccc67)c5c4)n3)cc21 ; c1ccc(-c2ccc(-c3ccc4c(c3)oc3cc(-n5c6ccccc6c6ccc(-c7nc(-c8ccccc8)nc(-c8ccccc8)n7)cc65)ccc34)cc2)cc1]

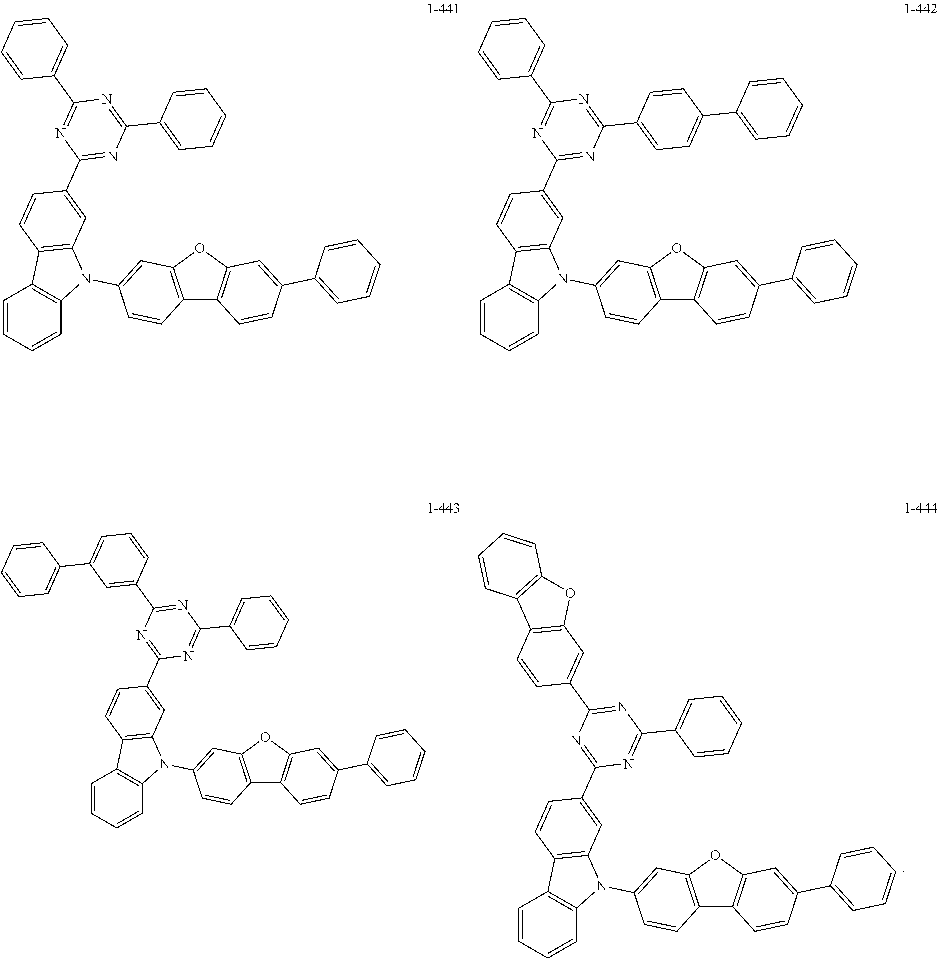 [SMILES: c1ccc(-c2ccc3c(c2)oc2cc(-n4c5ccccc5c5ccc(-c6nc(-c7ccccc7)nc(-c7ccccc7)n6)cc54)ccc23)cc1 ; c1ccc(-c2ccc(-c3nc(-c4ccccc4)nc(-c4ccc5c6ccccc6n(-c6ccc7c(c6)oc6cc(-c8ccccc8)ccc67)c5c4)n3)cc2)cc1 ; c1ccc(-c2cccc(-c3nc(-c4ccccc4)nc(-c4ccc5c6ccccc6n(-c6ccc7c(c6)oc6cc(-c8ccccc8)ccc67)c5c4)n3)c2)cc1 ; c1ccc(-c2ccc3c(c2)oc2cc(-n4c5ccccc5c5ccc(-c6nc(-c7ccccc7)nc(-c7ccc8c(c7)oc7ccccc78)n6)cc54)ccc23)cc1]

6. An organic light emitting device comprising:
a first electrode;
a second electrode provided opposite to the first electrode; and
one or more organic material layers provided between the first electrode and the second electrode,
wherein one or more layers of the one or more organic material layers comprise the heterocyclic compound of claim 1.

7. The organic light emitting device of claim 6, wherein the one or more organic material layer comprise a light emitting layer, and the light emitting layer includes the heterocyclic compound.

8. The organic light emitting device of claim 6, wherein the one or more organic material layer comprise a light emitting layer, the light emitting layer includes a host material, and the host material includes the heterocyclic compound.

9. The organic light emitting device of claim 6, wherein the one or more organic material layers further includes a heterocyclic compound represented by the following Chemical Formula 2: 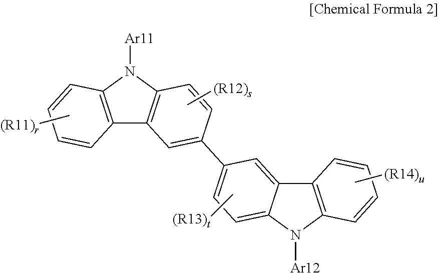
in Chemical Formula 2,
R11 to R14 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; and —NR201R202, or two or more groups adjacent to each other bond to each other to form a substituted or unsubstituted C6 to C60 aromatic hydrocarbon ring or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group;
Ar11 and Ar12 are the same as or different from each other, and each independently a substituted or unsubstituted C6 to C60 aryl group; or a substituted or unsubstituted C2 to C60 heteroaryl group;
r is an integer of 0 to 4, and when r is 2 or greater, R11s are the same as or different from each other;
s is an integer of 0 to 3, and when s is 2 or greater, R12s are the same as or different from each other;
t is an integer of 0 to 3, and when t is 2 or greater, R13s are the same as or different from each other; and
u is an integer of 0 to 4, and when u is 2 or greater, R14s are the same as or different from each other.

10. The organic light emitting device of claim 9, wherein the heterocyclic compound represented by Chemical Formula 2 does not include deuterium as a substituent, or has a deuterium content of 1% to 100% with respect to a total number of hydrogen atoms and deuterium atoms.

11. The organic light emitting device of claim 9, wherein at least one of the heterocyclic compound represented by Chemical Formula 1 and the heterocyclic compound represented by Chemical Formula 2 does not include deuterium as a substituent, or has a deuterium content of 1% to 100% with respect to a total number of hydrogen atoms and deuterium atoms.

12. The organic light emitting device of claim 9, wherein the heterocyclic compound represented by Chemical Formula 2 is any one selected from among the following compounds:

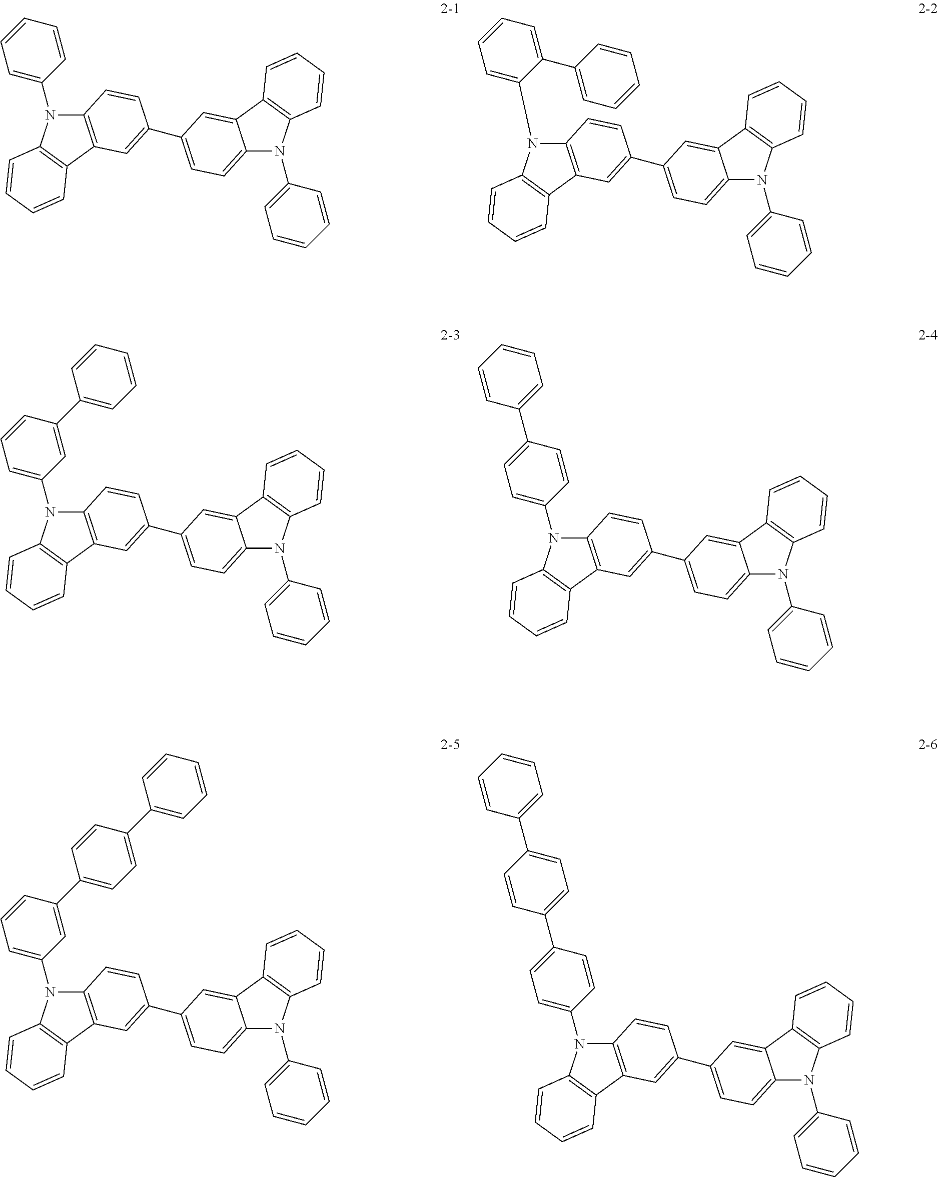 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

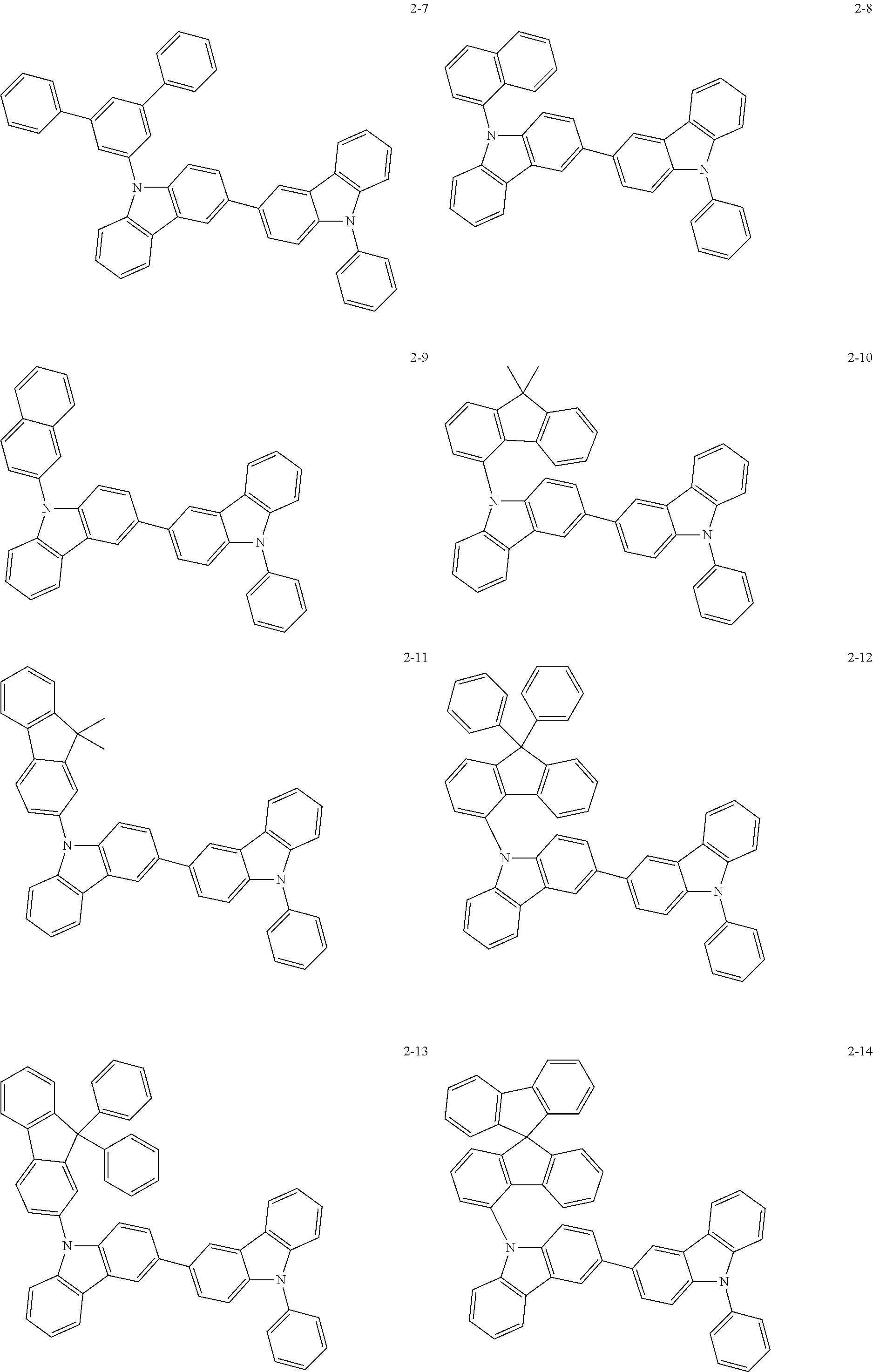

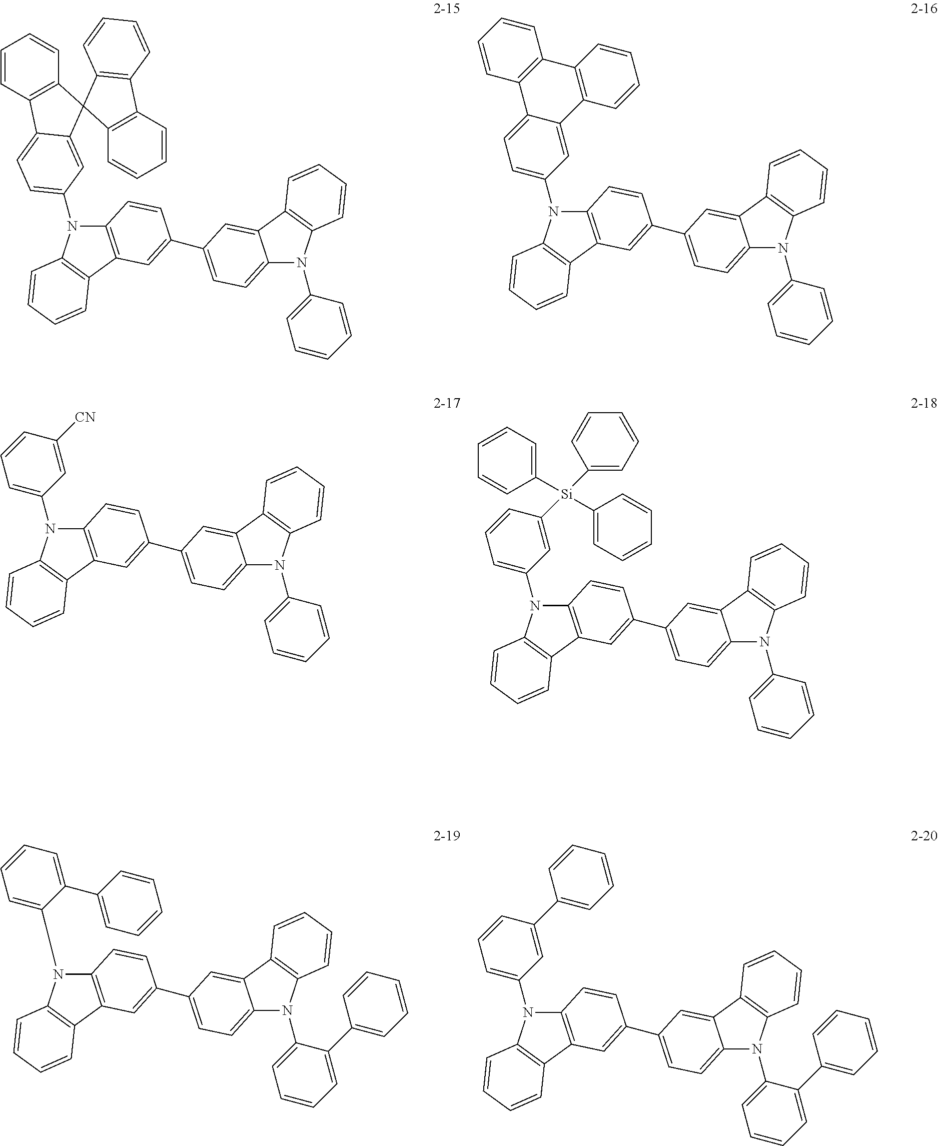

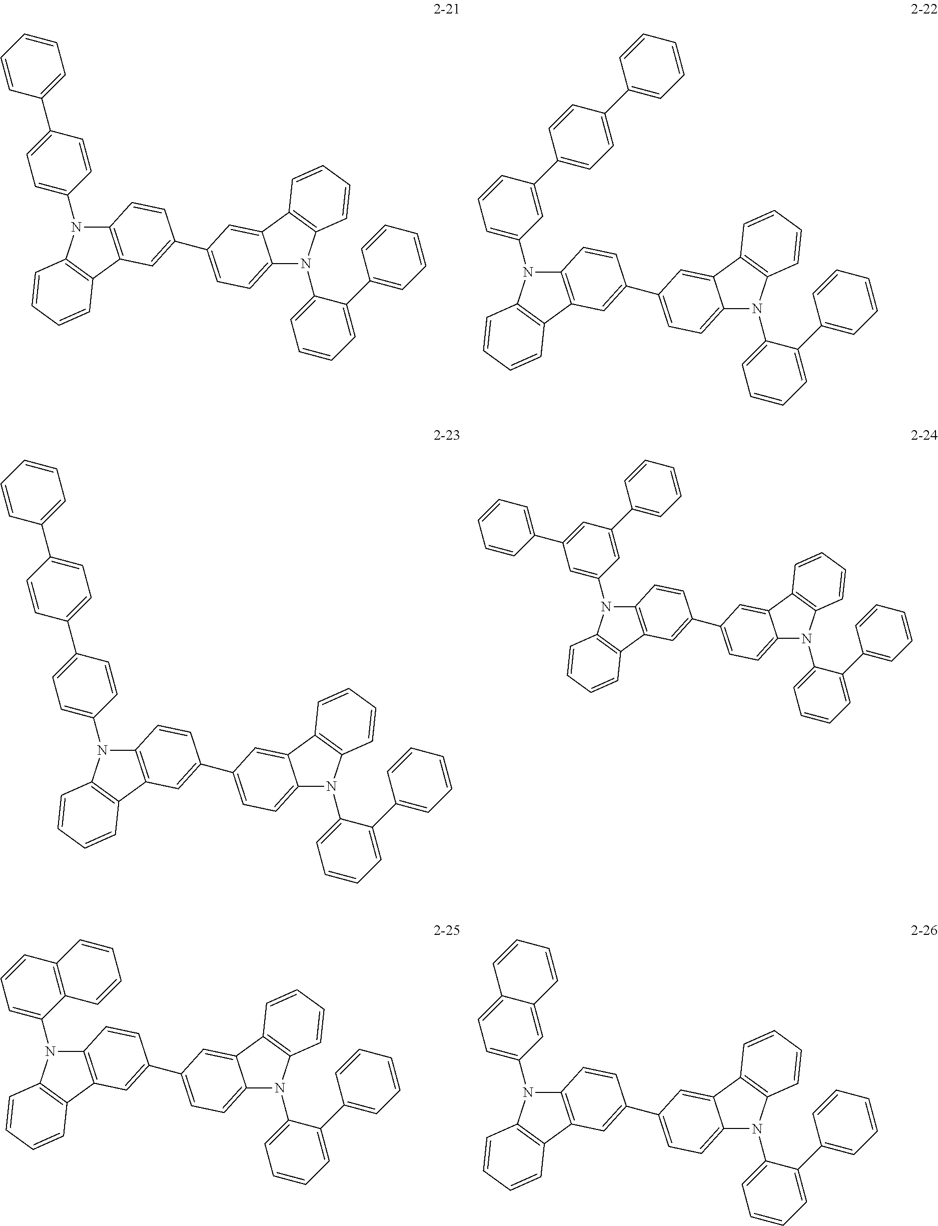

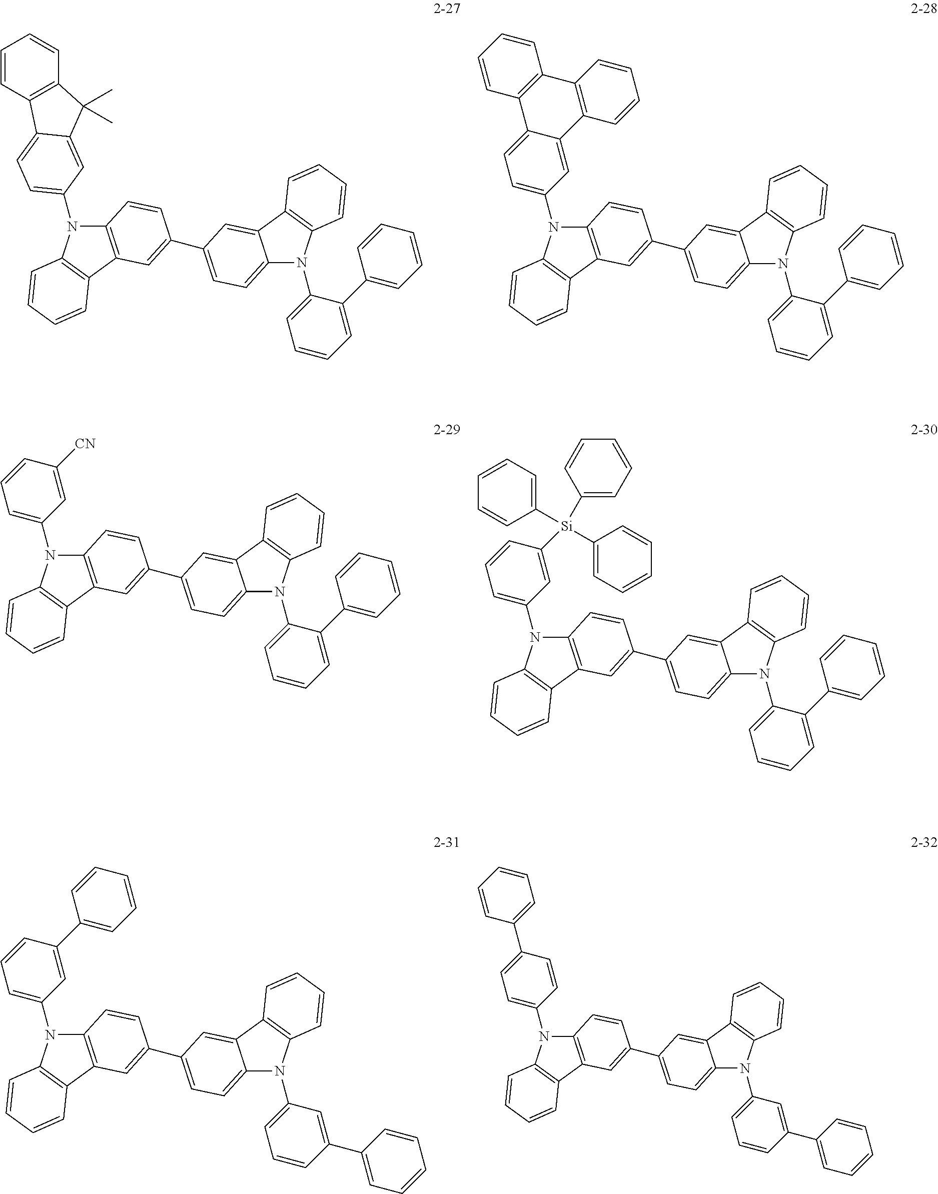

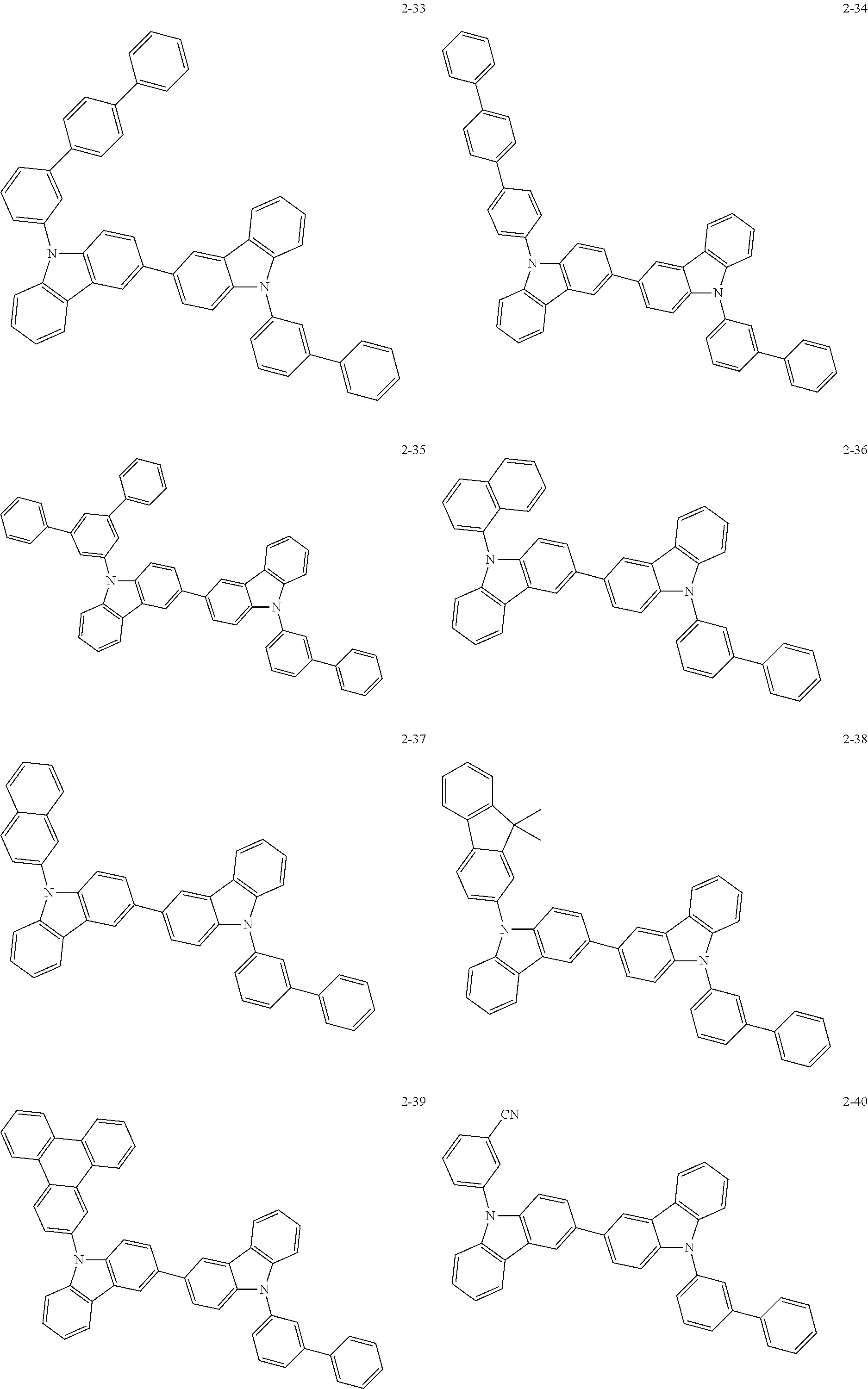

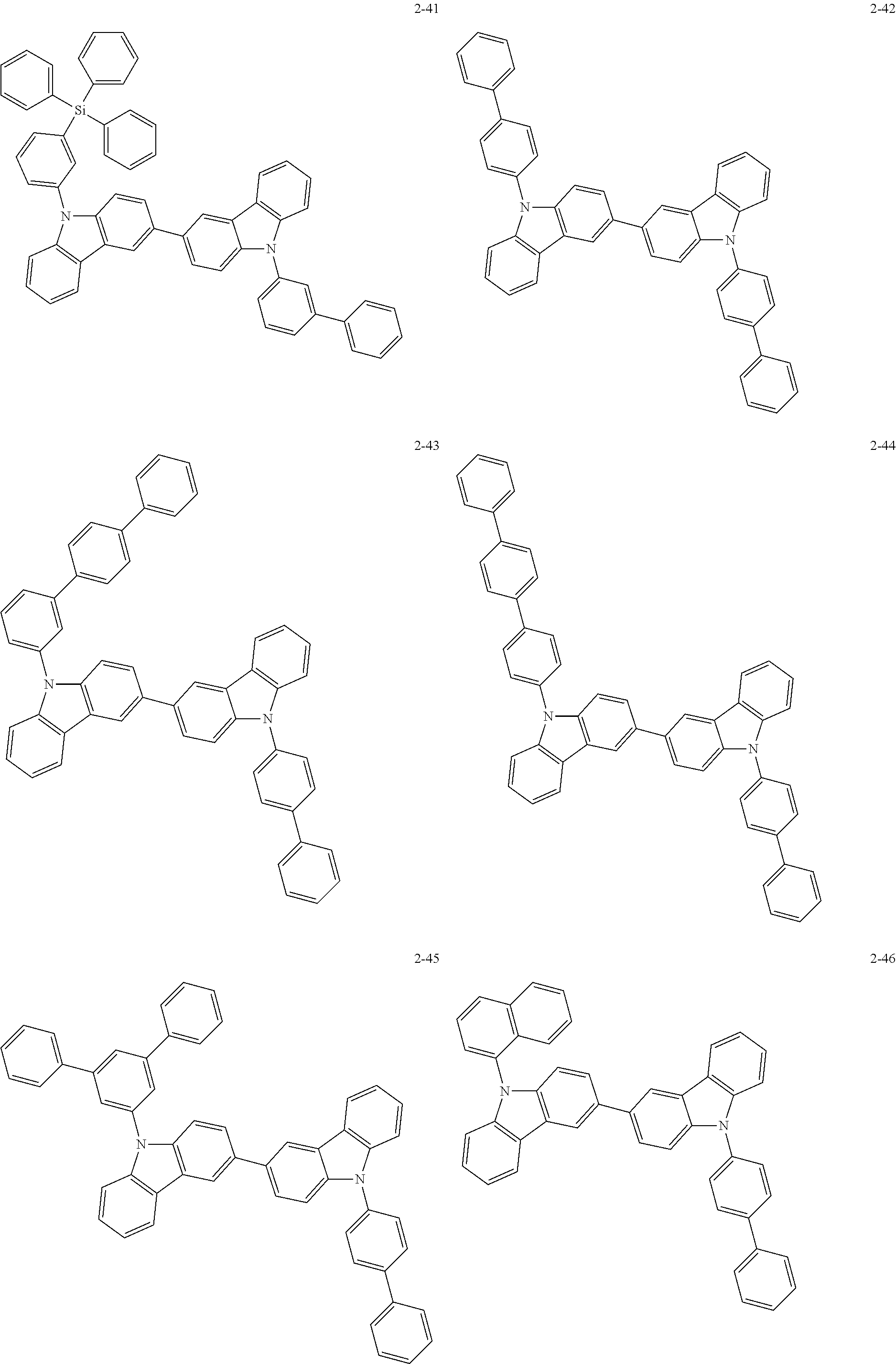

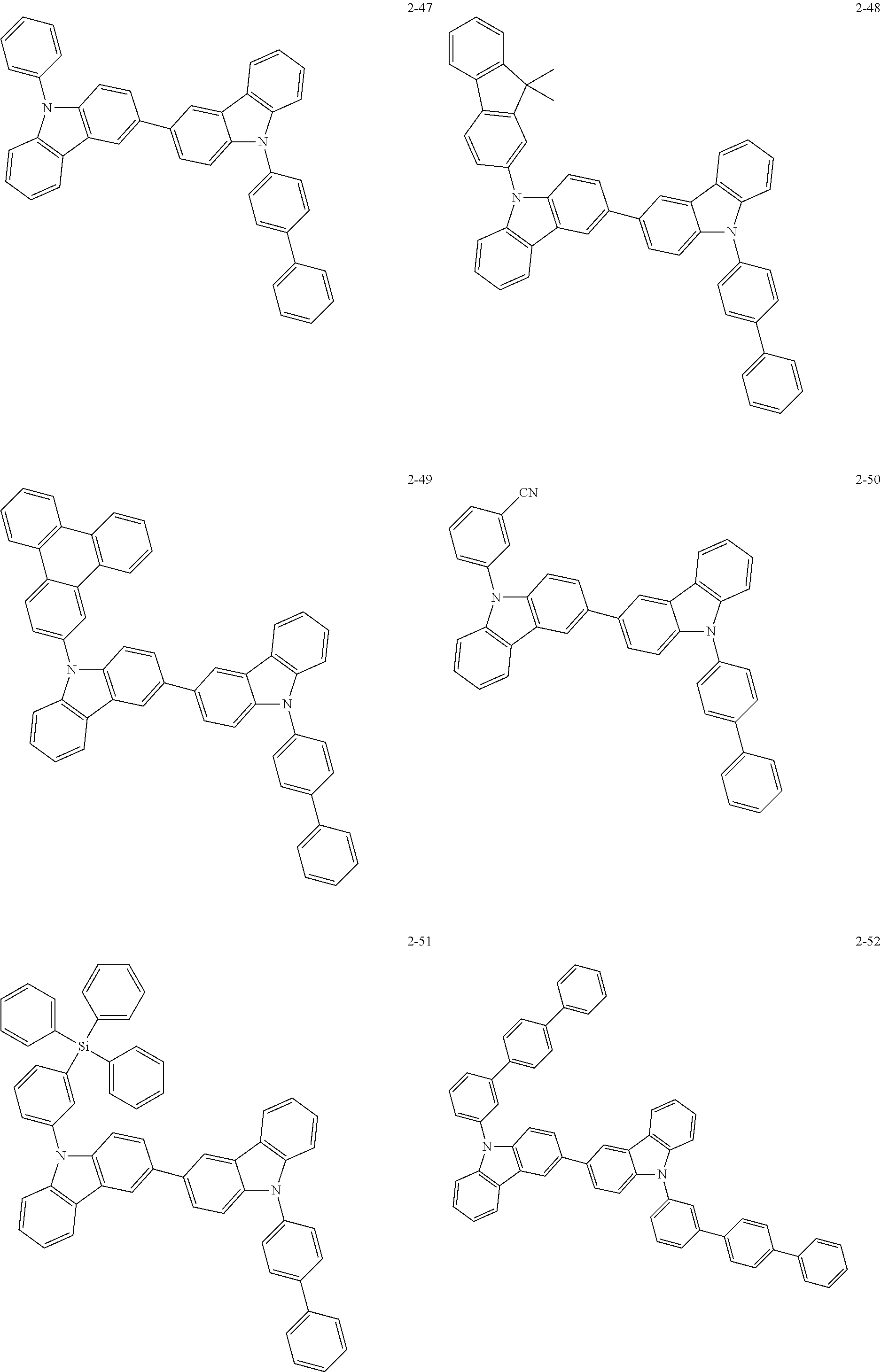

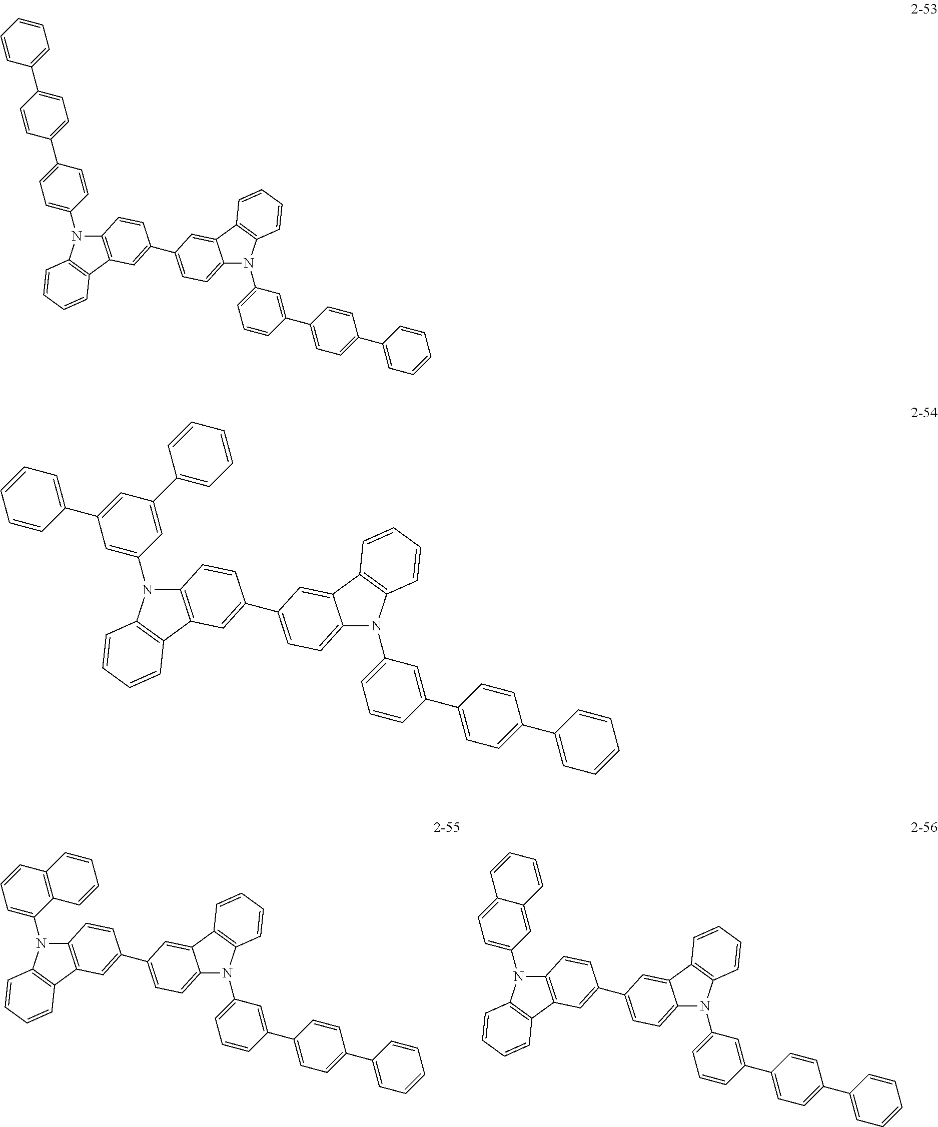 [SMILES: c1ccc(-c2ccc(-c3ccc(-n4c5ccccc5c5cc(-c6ccc7c(c6)c6ccccc6n7-c6cccc(-c7ccc(-c8ccccc8)cc7)c6)ccc54)cc3)cc2)cc1 ; c1ccc(-c2ccc(-c3cccc(-n4c5ccccc5c5cc(-c6ccc7c(c6)c6ccccc6n7-c6cc(-c7ccccc7)cc(-c7ccccc7)c6)ccc54)c3)cc2)cc1 ; c1ccc(-c2ccc(-c3cccc(-n4c5ccccc5c5cc(-c6ccc7c(c6)c6ccccc6n7-c6cccc7ccccc67)ccc54)c3)cc2)cc1 ; c1ccc(-c2ccc(-c3cccc(-n4c5ccccc5c5cc(-c6ccc7c(c6)c6ccccc6n7-c6ccc7ccccc7c6)ccc54)c3)cc2)cc1]

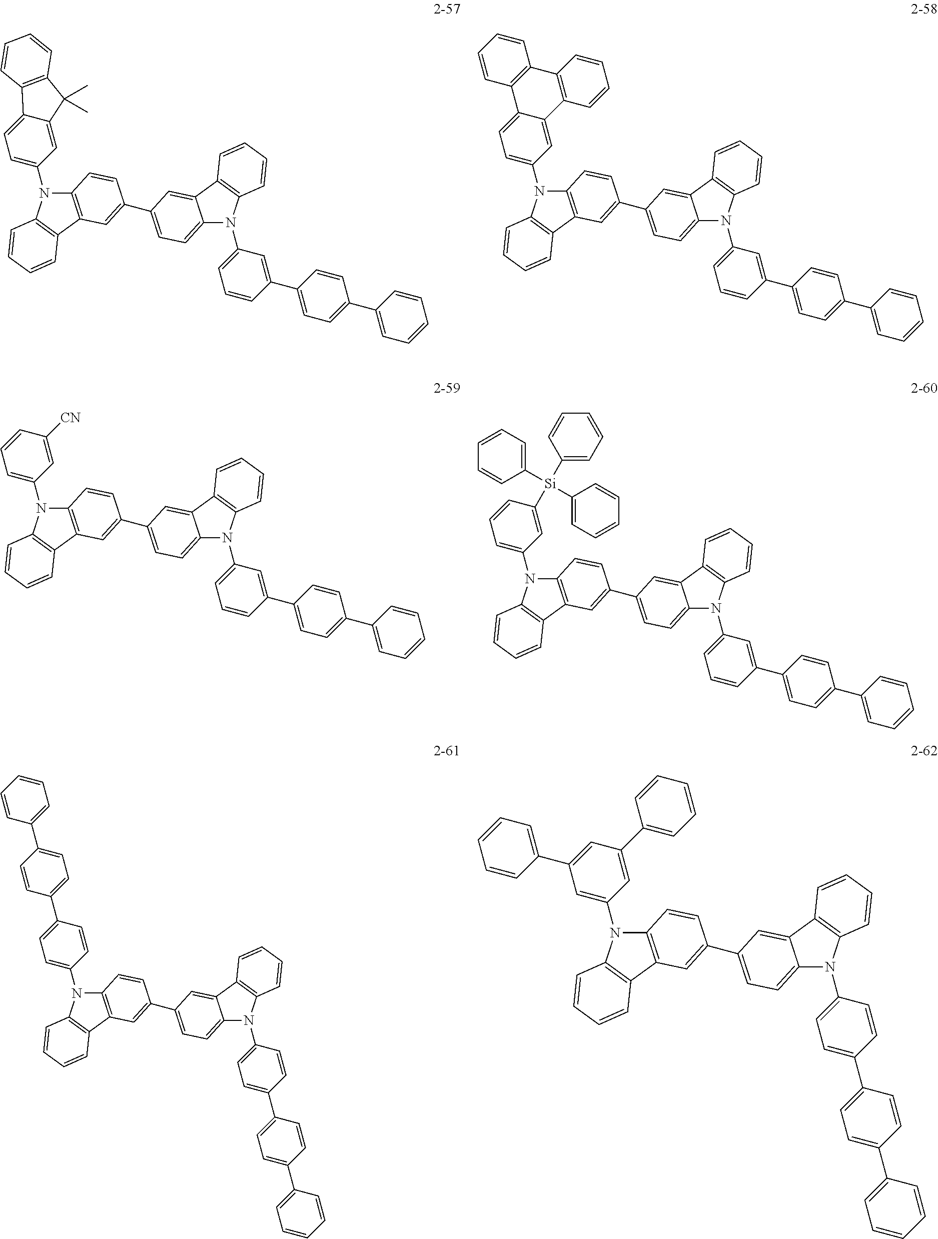

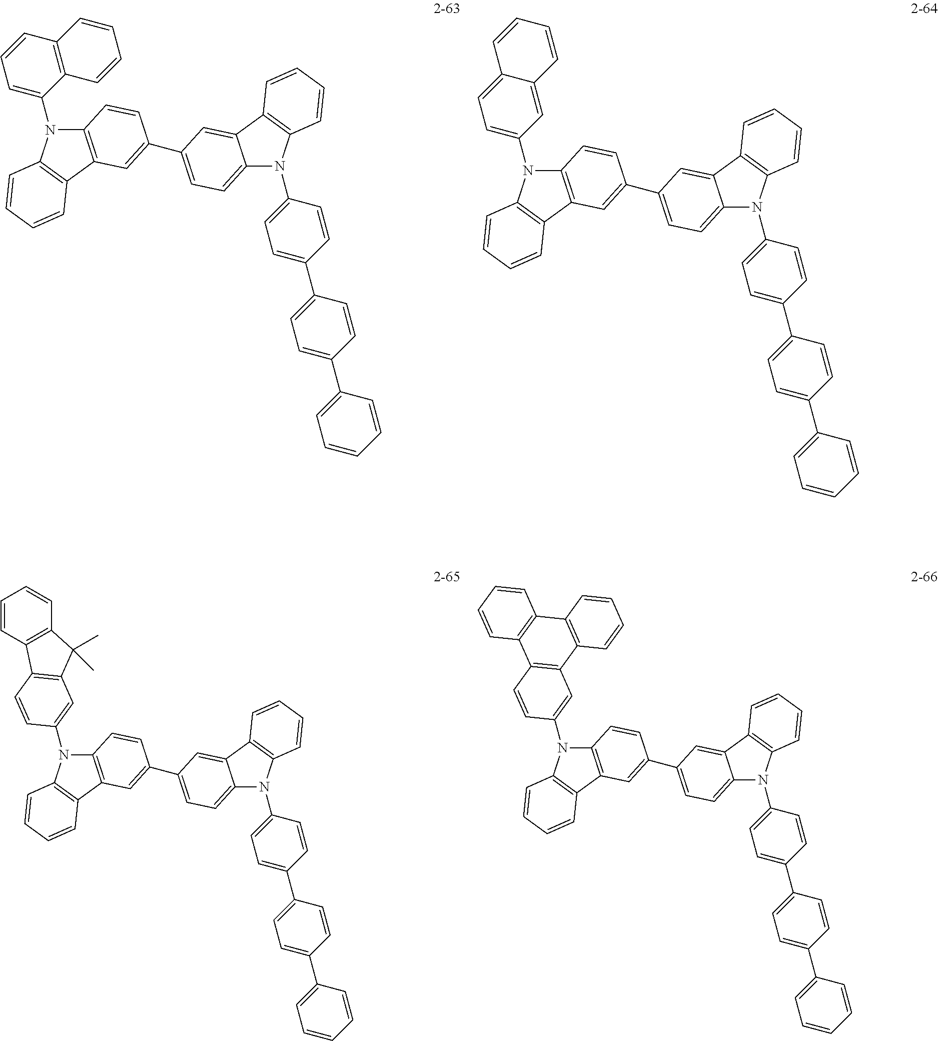 [SMILES: c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1 ; CC1(C)c2ccccc2-c2ccc(-n3c4ccccc4c4cc(-c5ccc6c(c5)c5ccccc5n6-c5ccc(-c6ccc(-c7ccccc7)cc6)cc5)ccc43)cc21 ; c1ccc(-c2ccc(-c3ccc(-n4c5ccccc5c5cc(-c6ccc7c(c6)c6ccccc6n7-c6ccc7c8ccccc8c8ccccc8c7c6)ccc54)cc3)cc2)cc1]

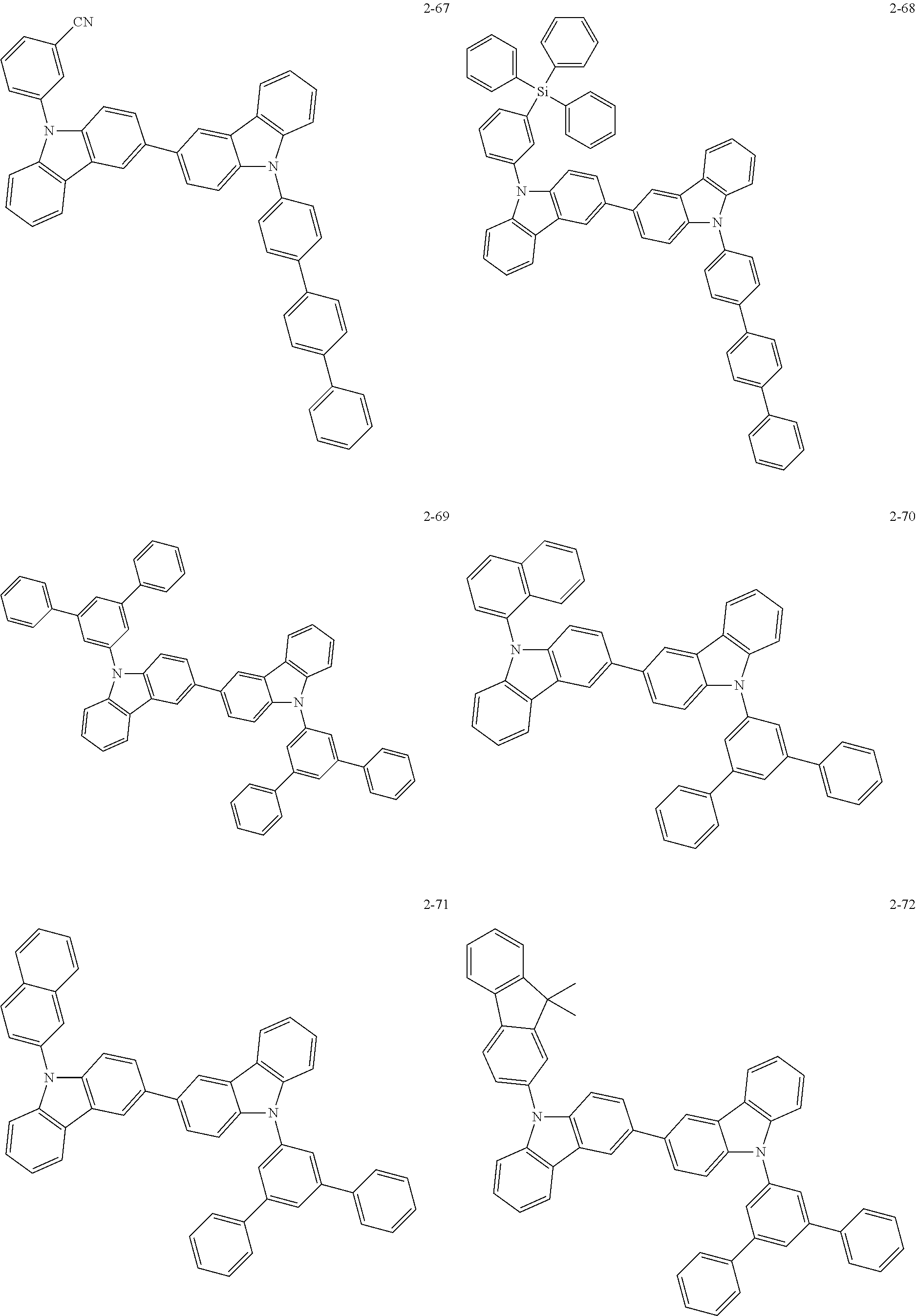

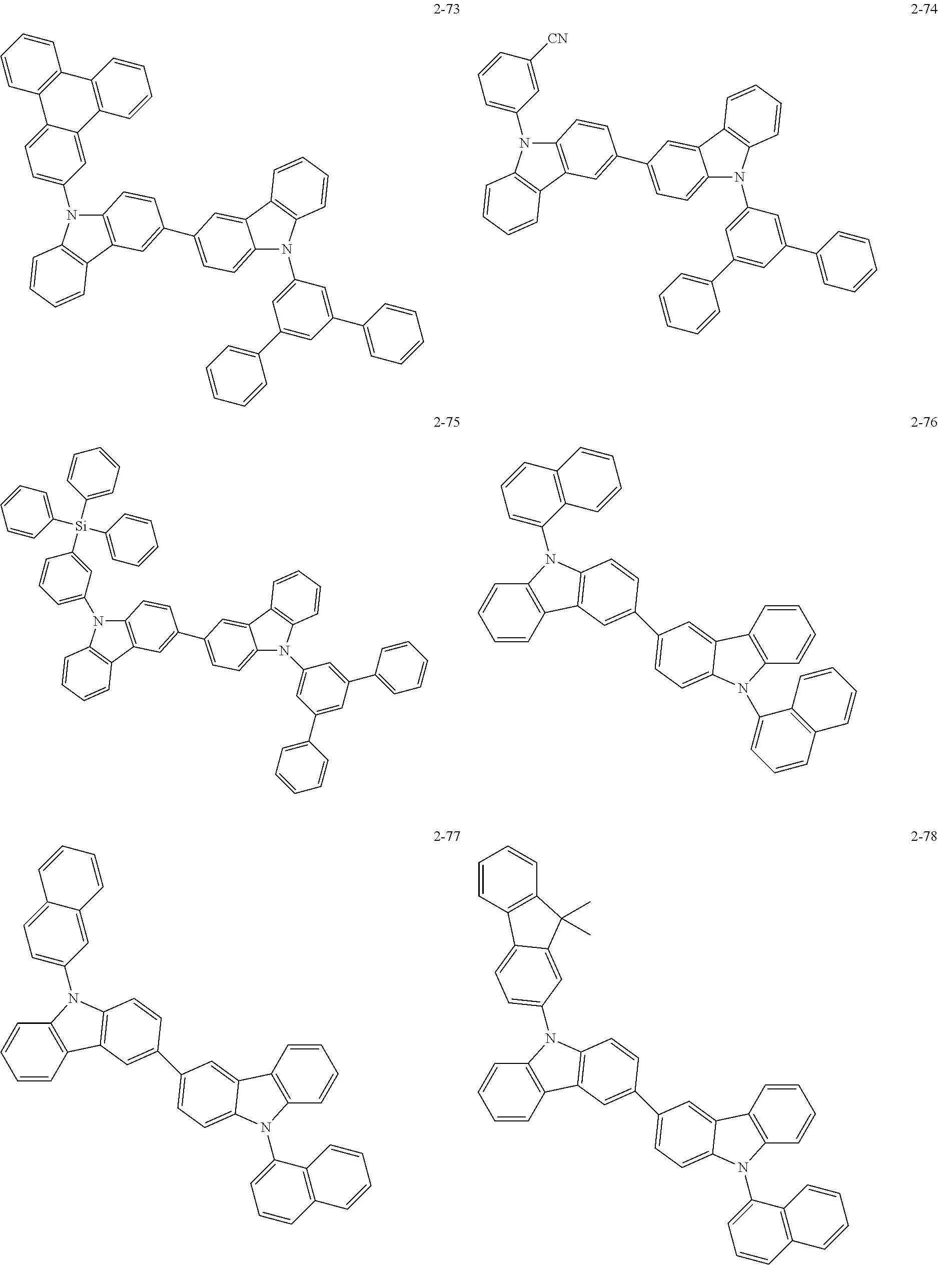

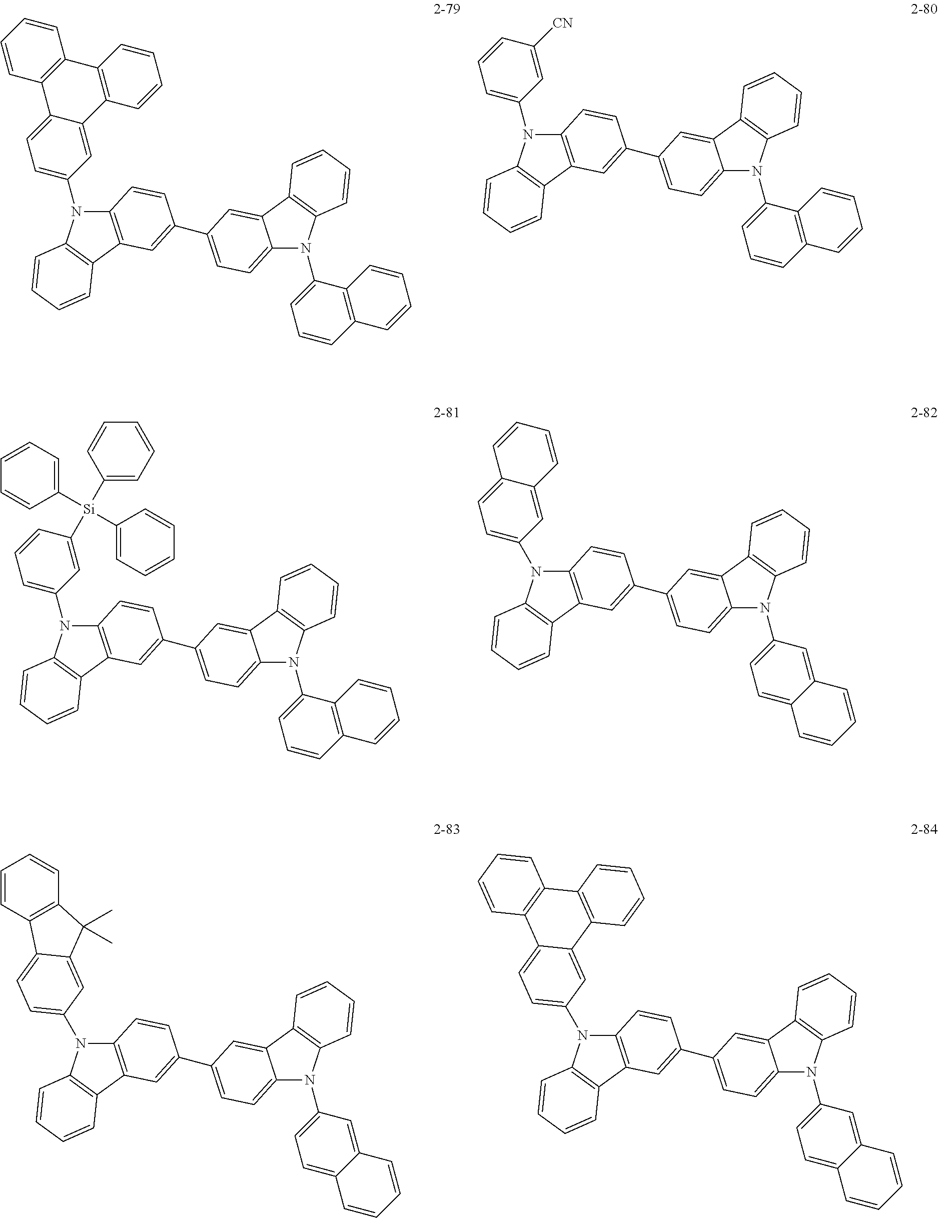

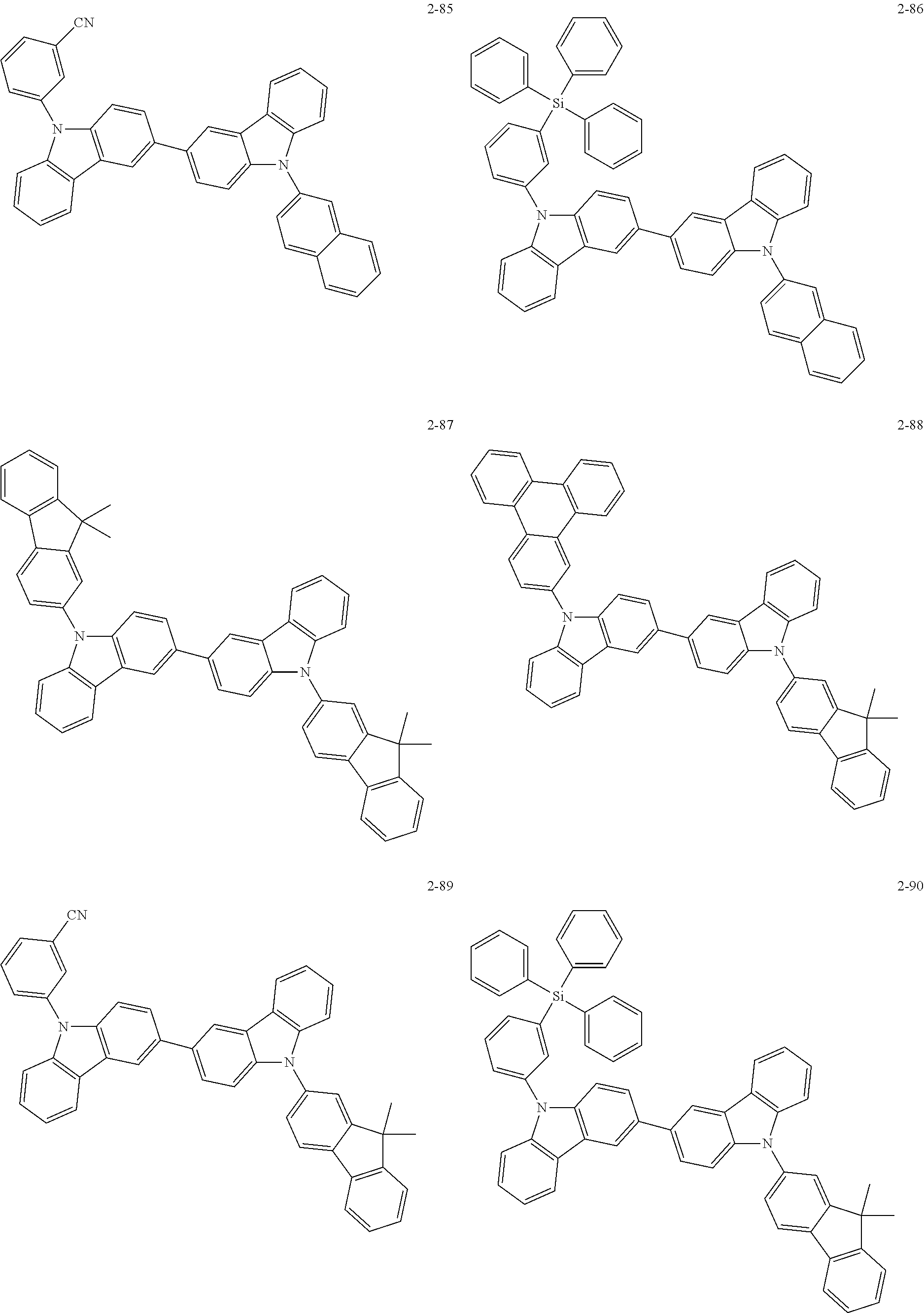

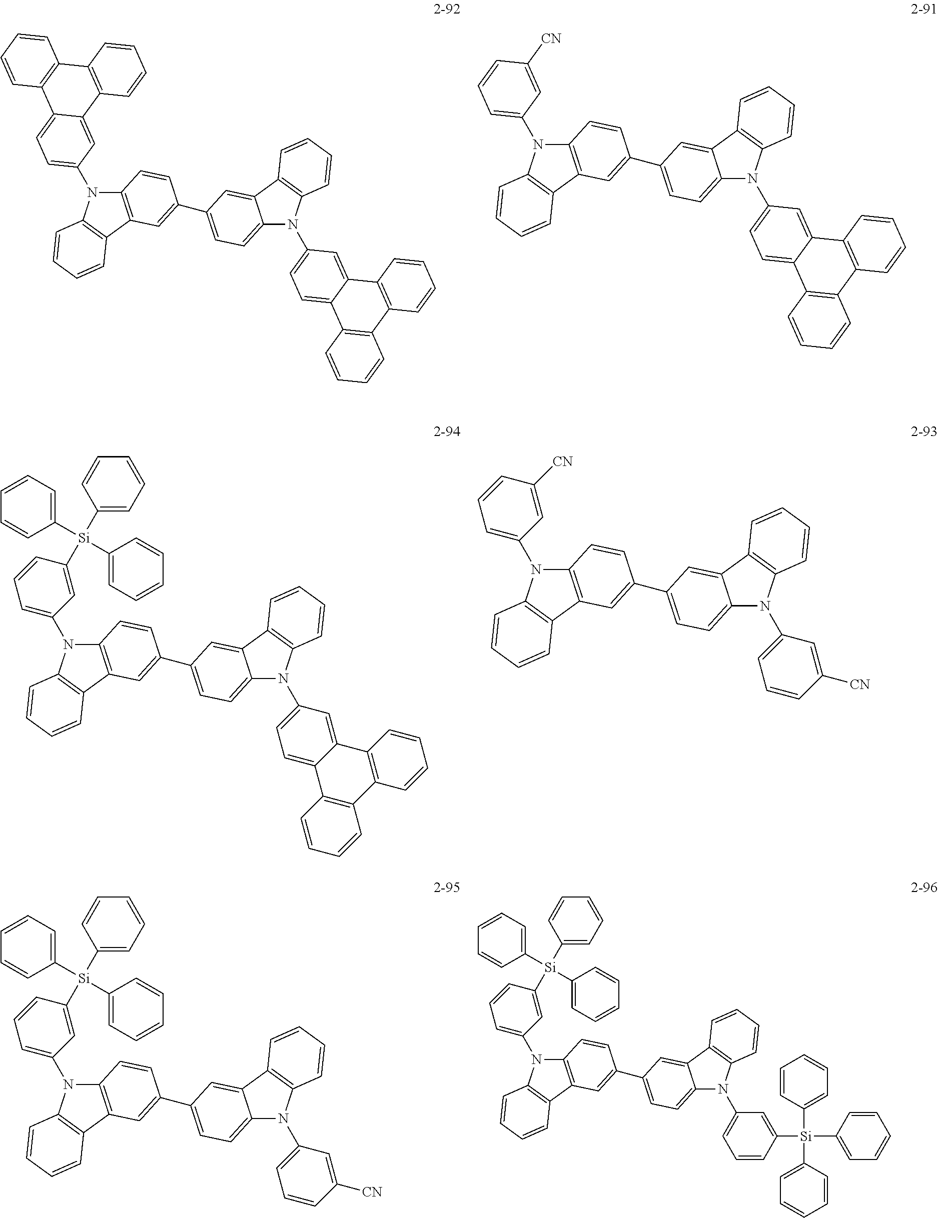

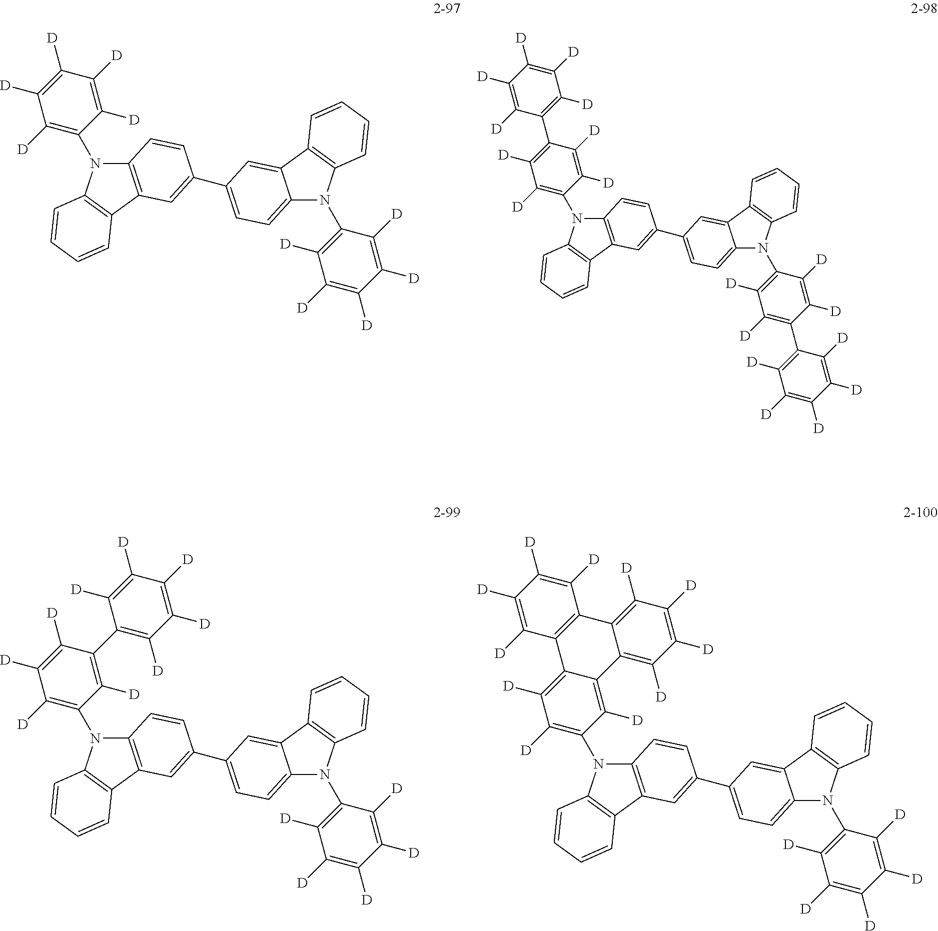

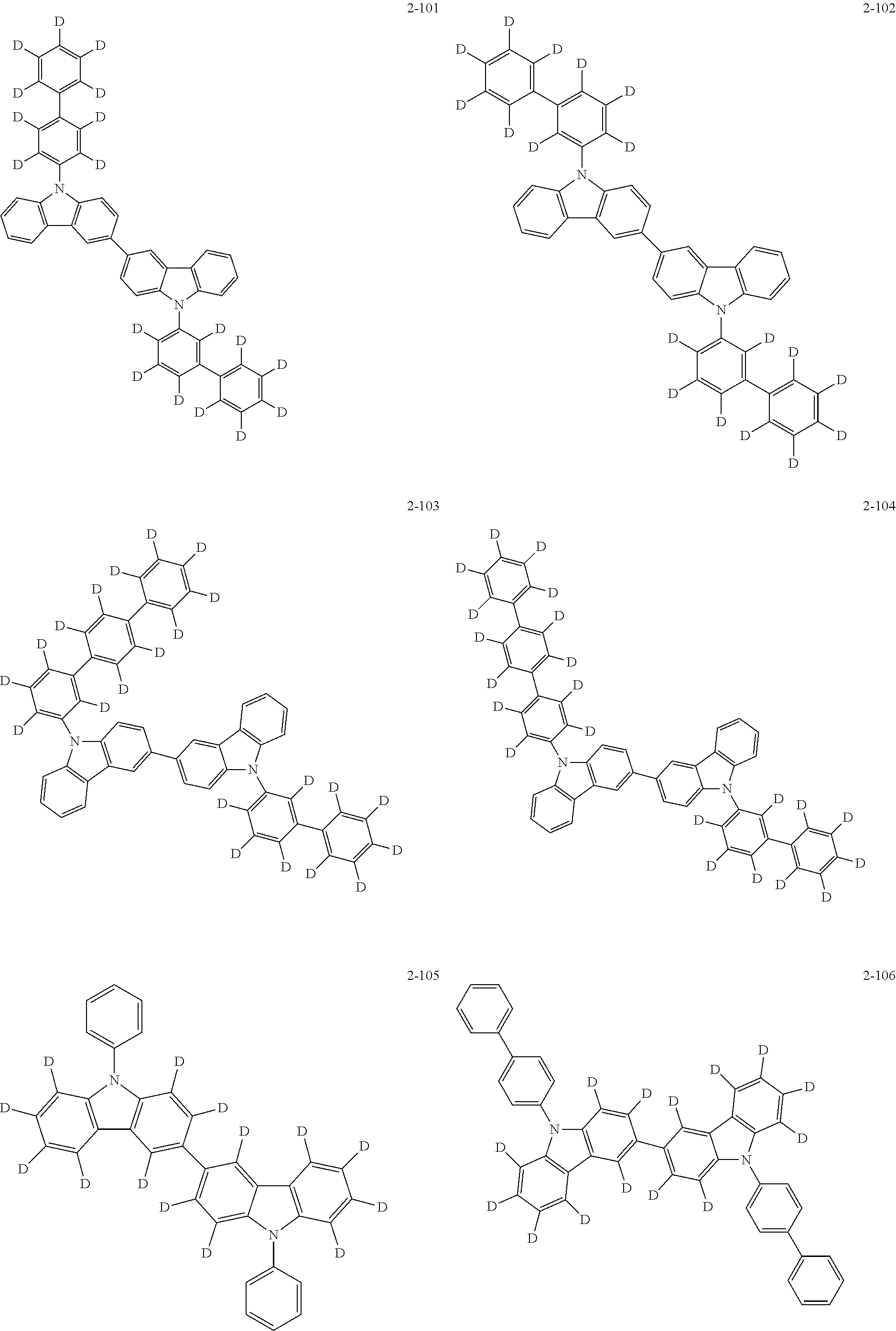

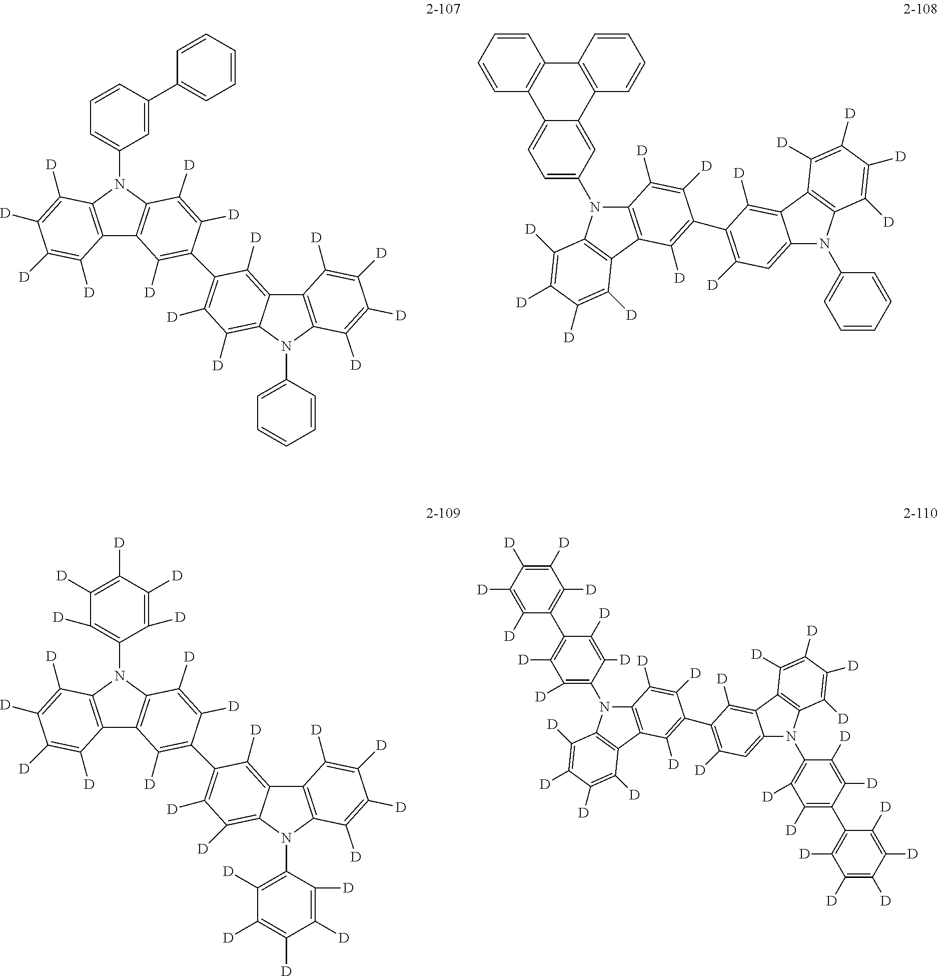

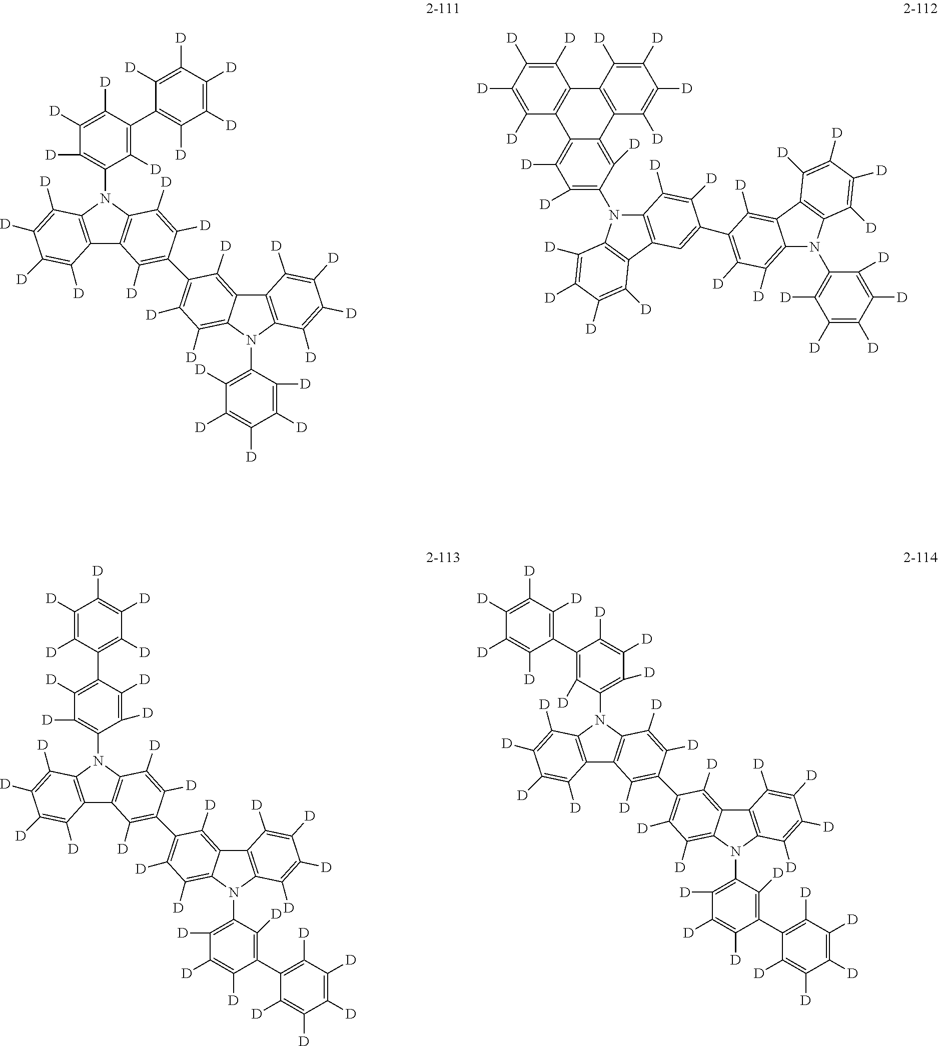

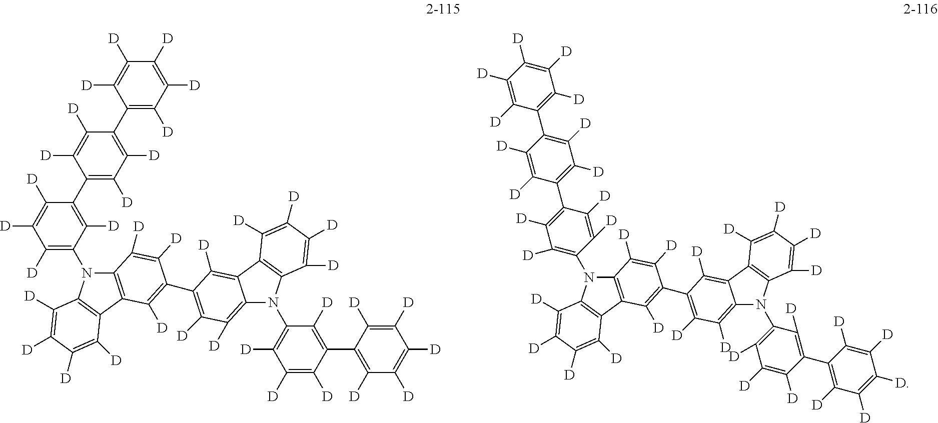

13. The organic light emitting device of claim 6, further comprising one, two or more layers selected from the group consisting of a light emitting layer, a hole injection layer, a hole transport layer, a hole transport auxiliary layer, an electron injection layer, an electron transport layer, an electron blocking layer and a hole blocking layer.

14. A composition for an organic material layer, the composition comprising:
the heterocyclic compound of claim 1; and
a heterocyclic compound represented by the following Chemical Formula 2: 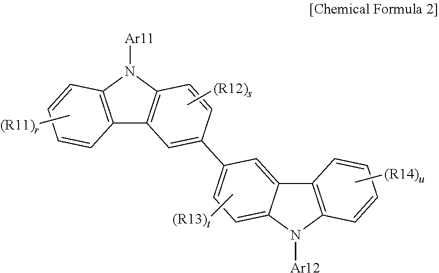
wherein, in Chemical Formula 2,
R11 to R14 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; and —NR201R202, or two or more groups adjacent to each other bond to each other to form a substituted or unsubstituted C6 to C60 aromatic hydrocarbon ring or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group;
Ar11 and Ar12 are the same as or different from each other, and each independently a substituted or unsubstituted C6 to C60 aryl group; or a substituted or unsubstituted C2 to C60 heteroaryl group;
r is an integer of 0 to 4, and when r is 2 or greater, R11s are the same as or different from each other;
s is an integer of 0 to 3, and when s is 2 or greater, R12s are the same as or different from each other;
t is an integer of 0 to 3, and when t is 2 or greater, R13s are the same as or different from each other; and
u is an integer of 0 to 4, and when u is 2 or greater, R14s are the same as or different from each other.

15. The composition of claim 14, wherein the heterocyclic compound represented by Chemical Formula 1 and the heterocyclic compound represented by Chemical Formula 2 have a weight ratio of 1:9 to 9:1.
